(19) United States
(12) Patent Application Publication
(10) Pub. No.: US 2021/0317102 A1
(43) Pub. Date: Oct. 14, 2021

(54) INHIBITORS OF INTEGRATED STRESS RESPONSE PATHWAY

(71) Applicant: Praxis Biotech LLC, San Francisco, CA (US)

(72) Inventors: Luz Marina DELGADO OYARZO, Santiago (CL); Gonzalo Andrés URETA DÍAZ, Santiago (CL); Brahmam PUJALA, Greater Noida (IN); Dayanand PANPATIL, Noida (IN); Sebastian BERNALES, Piedmont, CA (US); Sarvajit CHAKRAVARTY, Edmond, OK (US)

(21) Appl. No.: 16/899,520

(22) Filed: Jun. 11, 2020

Related U.S. Application Data
(60) Provisional application No. 62/943,666, filed on Dec. 4, 2019.
(60) Provisional application No. 62/860,676, filed on Jun. 12, 2019.

(51) Int. Cl. C07D 401/12; C07D 471/08; C07D 235/24; C07D 211/34; C07D 405/12; C07C 235/36; C07D 215/56; C07D 307/85; C07D 213/81; C07D 241/24; C07D 407/12; C07D 265/36; A01N 43/42; A01N 43/52; A01N 43/12; A01N 43/60; A01N 47/32; A01N 37/30; A01N 37/26; A01N 43/84; C12P 21/00

(52) U.S. Cl. CPC C07D 401/12; A61K 45/06; C07D 235/24; C07D 211/34; C07D 405/12; C07C 235/36; C07D 215/56; C07D 307/85; C07D 213/81; C07D 241/24; C07D 407/12; C07D 265/36; A01N 43/42; A01N 43/52; A01N 43/12; A01N 43/60; A01N 47/32; A01N 37/30; A01N 37/26; A01N 43/84; C12P 21/00; C07D 471/08

(57) ABSTRACT

The present disclosure relates generally to therapeutic agents that may be useful as inhibitors of Integrated Stress Response (ISR) pathway.

### CROSS REFERENCE TO RELATED APPLICATIONS

This application claims the priority benefit of U.S. Provisional Patent Application Nos. 62/860,676, filed Jun. 12, 2019, and 62/943,666, filed Dec. 4, 2019, the disclosures of which are hereby incorporated herein by reference in their entireties.

### FIELD

The present disclosure relates generally to therapeutic agents that may be useful as inhibitors of Integrated Stress Response (ISR) pathway.

### BACKGROUND

Genetically modifying plants to express heterologous proteins or increase the expression of endogenous proteins has become an important tool for a large number of business. Plants can be modified to express an increased amount of essential amino acids, to achieve greater yields of the plants or the proteins express therein, or to produce recombinant proteins such as biopolymers, industrial proteins/enzymes, and therapeutic proteins. However, there is a need to further increase the expression of plant proteins, which may require methods other than genetic modification.

In addition, given the resistance to genetically modifying plants by some people, it may be desirable to increase protein production in plants using other methods. Increased protein production by plants will likely be essential for ensuring the availability of enough protein to feed an increasing world population under changing environmental conditions. Further, increased protein production in plants promote plant growth, because additional proteins can be released through the roots into the surrounding area to attract microorganisms, such as bacteria that can in turn improve plant development.

One potential method of increasing protein production in plants is by modulating Integrated Stress Response (ISR) pathway. Diverse cellular conditions and stresses activate this widely conserved signaling pathway. The ISR pathway is activated in response to intrinsic and extrinsic stresses, such as viral infections, hypoxia, glucose and amino acid deprivation, oncogene activation, UV radiation, and endoplasmic reticulum stress. Upon activation of ISR by one or more of these factors, the eukaryotic initiation factor 2 (eIF2, which is comprised of three subunits, α, β and γ) becomes phosphorylated in its α-subunit and rapidly reduces overall protein translation by binding to the eIF2B complex. This phosphorylation inhibits the eIF2B-mediated exchange of GDP for GTP (i.e., a guanine nucleotide exchange factor (GEF) activity), sequestering eJF2B in a complex with eIF2 and reducing general protein translation of most mRNA in the cell. Paradoxically, eIF2a phosphorylation also increases translation of a subset of mRNAs that contain one or more upstream open reading frames (uORFs) in their 5′ untranslated region (UTR). These transcripts include the transcriptional modulator activating transcription factor 4 (ATF4), the transcription factor CHOP, the growth arrest and DNA damage-inducible protein GADD34 and the β-secretase BACE-1.

Additionally, compounds useful in modulating the ISR pathway may also be useful in treating a large number of diseases. In animals, the ISR pathway modulates a broad translational and transcriptional program involved in diverse processes such as learning memory, immunity, intermediary metabolism, insulin production and resistance to unfolded protein stress in the endoplasmic reticulum, among others. Activation of the ISR pathway has also been associated with numerous pathological conditions including cancer, neurodegenerative diseases, metabolic diseases (metabolic syndrome), autoimmune diseases, inflammatory diseases, musculoskeletal diseases (such as myopathy), vascular diseases, ocular diseases, and genetic disorders. Aberrant protein synthesis through eIF2a phosphorylation is also characteristic of several other human genetic disorders, cystic fibrosis, amyotrophic lateral sclerosis, Huntington disease and prion disease.

### BRIEF SUMMARY

Inhibitors of the Integrated Stress Response (ISR) pathway are described, as are methods of making and using the compounds, or salts thereof.

### BRIEF DESCRIPTION OF THE DRAWINGS

FIG. 1A shows percent of protein synthesis in mouse quadriceps from fed or fasted animals treated with vehicle or compound 11.

FIG. 1B shows percent of protein synthesis in mouse gastrocnemius from fed or fasted animals treated with vehicle or compound 11.

FIG. 1C shows percent of protein synthesis in mouse tibialis anterior from fed or fasted animals treated with vehicle or compound 11.

FIG. 1D shows expression of the muscle atrophy marker MuRF-1 in quadriceps from fed or fasted mice treated with vehicle or compound 11.

FIG. 2 shows relative fluorescence intensity (RFU) of GFP treated with either vehicle or test compounds 15, 17, 20, 23, 26, 95, 96, or 97 in a cell-free expression system.

FIG. 3A shows total protein secretion in CHO cells treated with vehicle or with 1 μM of compound 10.

FIG. 3B shows the percentage of total protein secretion in CHO cells treated with vehicle or 1 μM compound 10.

FIG. 4 shows the percentage of secreted Ig kappa light chain by ARH cells treated with vehicle or compound 10 from three independent experiments.

FIG. 5 shows the percentage of secreted Wnt-3A by L-Wnt3A cells treated with vehicle or compound 10 from two independent experiments.

FIG. 6 shows the amount of secreted human EGF protein by Saccharomyces cerevisiae stable expressing the recombinant human EGF protein treated with either vehicle or 1 μM test compounds 10, 25, or 33.

### DETAILED DESCRIPTION

### Definitions

For use herein, unless clearly indicated otherwise, use of the terms “a”, “an” and the like refers to one or more.

Reference to “about” a value or parameter herein includes (and describes) embodiments that are directed to that value or parameter per se. For example, description referring to “about X” includes description of “X”.

“Alkyl” as used herein refers to and includes, unless otherwise stated, a saturated linear (i.e., unbranched) or branched univalent hydrocarbon chain or combination thereof, having the number of carbon atoms designated (i.e., C₁-C₁₀ means one to ten carbon atoms). Particular alkyl groups are those having 1 to 20 carbon atoms (a “C₁-C₂₀ alkyl”), having 1 to 10 carbon atoms (a “C₁-C₁₀ alkyl”), having 6 to 10 carbon atoms (a “C₆-C₁₀ alkyl”), having 1 to 6 carbon atoms (a “C₁-C₆ alkyl”), having 2 to 6 carbon atoms (a “C₂-C₆ alkyl”), or having 1 to 4 carbon atoms (a “C₁-C₄ alkyl”). Examples of alkyl groups include, but are not limited to, groups such as methyl, ethyl, n-propyl, isopropyl, n-butyl, t-butyl, isobutyl, sec-butyl, n-pentyl, n-hexyl, n-heptyl, n-octyl, n-nonyl, n-decyl, and the like.

“Alkylene” as used herein refers to the same residues as alkyl, but having bivalency. Particular alkylene groups are those having 1 to 20 carbon atoms (a “C₁-C₂₀ alkylene”), having 1 to 10 carbon atoms (a “C₁-C₁₀ alkylene”), having 6 to 10 carbon atoms (a “C₆-C₁₀ alkylene”), having 1 to 6 carbon atoms (a “C₁-C₆ alkylene”), 1 to 5 carbon atoms (a “C₁-C₅ alkylene”), 1 to 4 carbon atoms (a “C₁-C₄ alkylene”) or 1 to 3 carbon atoms (a “C₁-C₃ alkylene”). Examples of alkylene include, but are not limited to, groups such as methylene (—CH₂—), ethylene (—CH₂CH₂—), propylene (—CH₂CH₂CH₂—), isopropylene (—CH₂CH(CH₃)—), butylene (—CH₂(CH₂)₂CH₂—), isobutylene (—CH₂CH(CH₃)CH₂—), pentylene (—CH₂(CH₂)₃CH₂—), hexylene (—CH₂(CH₂)₄CH₂—), heptylene (—CH₂(CH₂)₅CH₂—), octylene (—CH₂(CH₂)₆CH₂—), and the like.

“Alkenyl” as used herein refers to and includes, unless otherwise stated, an unsaturated linear (i.e., unbranched) or branched univalent hydrocarbon chain or combination thereof, having at least one site of olefinic unsaturation (i.e., having at least one moiety of the formula C═C) and having the number of carbon atoms designated (i.e., C₂-C₁₀ means two to ten carbon atoms). An alkenyl group may have “cis” or “trans” configurations, or alternatively have “E” or “Z” configurations. Particular alkenyl groups are those having 2 to 20 carbon atoms (a “C₂-C₂₀ alkenyl”), having 6 to 10 carbon atoms (a “C₆-C₁₀ alkenyl”), having 2 to 8 carbon atoms (a “C₂-C₈ alkenyl”), having 2 to 6 carbon atoms (a “C₂-C₆ alkenyl”), or having 2 to 4 carbon atoms (a “C₂-C₄ alkenyl”). Examples of alkenyl group include, but are not limited to, groups such as ethenyl (or vinyl), prop-1-enyl, prop-2-enyl (or allyl), 2-methylprop-1-enyl, but-1-enyl, but-2-enyl, but-3-enyl, buta-1,3-dienyl, 2-methylbuta-1,3-dienyl, pent-1-enyl, pent-2-enyl, hex-1-enyl, hex-2-enyl, hex-3-enyl, and the like.

“Alkenylene” as used herein refers to the same residues as alkenyl, but having bivalency. Particular alkenylene groups are those having 2 to 20 carbon atoms (a “C₂-C₂ alkenylene”), having 2 to 10 carbon atoms (a “C₂-C₁₀ alkenylene”), having 6 to 10 carbon atoms (a “C₁-C₁₀ alkenylene”), having 2 to 6 carbon atoms (a “C₂-C₆ alkenylene”), 2 to 4 carbon atoms (a “C₂-C₄ alkenylene”) or 2 to 3 carbon atoms (a “C₂-C₃ alkenylene”). Examples of alkenylene include, but are not limited to, groups such as ethenylene (or vinylene) (—CH═CH—), propenylene (—CH═CHCH₂—), 1,4-but-1-enylene (—CH═CH—CH₂CH₂—), 1,4-but-2-enylene (—CH₂CH═CHCH₂—), 1,6-hex-1-enylene (—CH═CH—(CH₂)₃CH₂—), and the like.

“Alkynyl” as used herein refers to and includes, unless otherwise stated, an unsaturated linear (i.e., unbranched) or branched univalent hydrocarbon chain or combination thereof, having at least one site of acetylenic unsaturation (i.e., having at least one moiety of the formula C═C) and having the number of carbon atoms designated (i.e., C₂-C₁₀ means two to ten carbon atoms). Particular alkynyl groups are those having 2 to 20 carbon atoms (a “C₂-C₂₀ alkynyl”), having 6 to 10 carbon atoms (a “C₆-C₁₀ alkynyl”), having 2 to 8 carbon atoms (a “C₂-C₈ alkynyl”), having 2 to 6 carbon atoms (a “C₂-C₆ alkynyl”), or having 2 to 4 carbon atoms (a “C₂-C₄ alkynyl”). Examples of alkynyl group include, but are not limited to, groups such as ethynyl (or acetylenyl), prop-1-ynyl, prop-2-ynyl (or propargyl), but-1-ynyl, but-2-ynyl, but-3-ynyl, and the like.

“Alkynylene” as used herein refers to the same residues as alkynyl, but having bivalency. Particular alkynylene groups are those having 2 to 20 carbon atoms (a “C₂-C₂₀ alkynylene”), having 2 to 10 carbon atoms (a “C₂-C₁₀ alkynylene”), having 6 to 10 carbon atoms (a “C₆-C₁₀ alkynylene”), having 2 to 6 carbon atoms (a “C₂-C₆ alkynylene”), 2 to 4 carbon atoms (a “C₂-C₄ alkynylene”) or 2 to 3 carbon atoms (a “C₂-C₃ alkynylene”). Examples of alkynylene include, but are not limited to, groups such as ethynylene (or acetylenylene) (—C≡C—), propynylene (—C≡CCH₂—), and the like.

“Cycloalkyl” as used herein refers to and includes, unless otherwise stated, saturated cyclic univalent hydrocarbon structures, having the number of carbon atoms designated (i.e., C₃-C₁₀ means three to ten carbon atoms). Cycloalkyl can consist of one ring, such as cyclohexyl, or multiple rings, such as adamantyl. A cycloalkyl comprising more than one ring may be fused, spiro or bridged, or combinations thereof. Particular cycloalkyl groups are those having from 3 to 12 annular carbon atoms. A preferred cycloalkyl is a cyclic hydrocarbon having from 3 to 8 annular carbon atoms (a “C₃-C₈ cycloalkyl”), having 3 to 6 carbon atoms (a “C₃-C₆ cycloalkyl”), or having from 3 to 4 annular carbon atoms (a “C₃-C₄ cycloalkyl”). Examples of cycloalkyl include, but are not limited to, cyclopropyl, cyclobutyl, cyclopentyl, cyclohexyl, cycloheptyl, norbornyl, and the like.

“Cycloalkylene” as used herein refers to the same residues as cycloalkyl, but having bivalency. Cycloalkylene can consist of one ring or multiple rings which may be fused, spiro or bridged, or combinations thereof. Particular cycloalkylene groups are those having from 3 to 12 annular carbon atoms. A preferred cycloalkylene is a cyclic hydrocarbon having from 3 to 8 annular carbon atoms (a “C₃-C₈ cycloalkylene”), having 3 to 6 carbon atoms (a “C₃-C₆ cycloalkylene”), or having from 3 to 4 annular carbon atoms (a “C₃-C₄ cycloalkylene”). Examples of cycloalkylene include, but are not limited to, cyclopropylene, cyclobutylene, cyclopentylene, cyclohexylene, cycloheptylene, norbornylene, and the like. A cycloalkylene may attach to the remaining structures via the same ring carbon atom or different ring carbon atoms. When a cycloalkylene attaches to the remaining structures via two different ring carbon atoms, the connecting bonds may be cis- or trans- to each other. For example, cyclopropylene may include 1,1-cyclopropylene and 1,2-cyclopropylene (e.g., cis-1,2-cyclopropylene or trans-1,2-cyclopropylene), or a mixture thereof.

“Cycloalkenyl” refers to and includes, unless otherwise stated, an unsaturated cyclic non-aromatic univalent hydrocarbon structure, having at least one site of olefinic unsaturation (i.e., having at least one moiety of the formula C═C) and having the number of carbon atoms designated (i.e., C₂-C₁₀ means two to ten carbon atoms). Cycloalkenyl can consist of one ring, such as cyclohexenyl, or multiple rings, such as norbornenyl. A preferred cycloalkenyl is an unsaturated cyclic hydrocarbon having from 3 to 8 annular carbon atoms (a “C₃-C₈ cycloalkenyl”). Examples of cycloalkenyl groups include, but are not limited to, cyclopropenyl, cyclobutenyl, cyclopentenyl, cyclohexenyl, norbornenyl, and the like.

“Cycloalkenylene” as used herein refers to the same residues as cycloalkenyl, but having bivalency.

“Aryl” or “Ar” as used herein refers to an unsaturated aromatic carbocyclic group having a single ring (e.g., phenyl) or multiple condensed rings (e.g., naphthyl or anthryl) which condensed rings may or may not be aromatic. Particular aryl groups are those having from 6 to 14 annular carbon atoms (a “C₆-C₁₄ aryl”). An aryl group having more than one ring where at least one ring is non-aromatic may be connected to the parent structure at either an aromatic ring position or at a non-aromatic ring position. In one variation, an aryl group having more than one ring where at least one ring is non-aromatic is connected to the parent structure at an aromatic ring position.

“Arylene” as used herein refers to the same residues as aryl, but having bivalency. Particular arylene groups are those having from 6 to 14 annular carbon atoms (a “C₆-C₁₄ arylene”).

“Heteroaryl” as used herein refers to an unsaturated aromatic cyclic group having from 1 to 14 annular carbon atoms and at least one annular heteroatom, including but not limited to heteroatoms such as nitrogen, oxygen, and sulfur. A heteroaryl group may have a single ring (e.g., pyridyl, furyl) or multiple condensed rings (e.g., indolizinyl, benzothienyl) which condensed rings may or may not be aromatic. Particular heteroaryl groups are 5 to 14-membered rings having 1 to 12 annular carbon atoms and 1 to 6 annular heteroatoms independently selected from nitrogen, oxygen, and sulfur, 5 to 10-membered rings having 1 to 8 annular carbon atoms and 1 to 4 annular heteroatoms independently selected from nitrogen, oxygen, and sulfur, or 5, 6 or 7-membered rings having 1 to 5 annular carbon atoms and 1 to 4 annular heteroatoms independently selected from nitrogen, oxygen, and sulfur. In one variation, particular heteroaryl groups are monocyclic aromatic 5-, 6- or 7-membered rings having from 1 to 6 annular carbon atoms and 1 to 4 annular heteroatoms independently selected from nitrogen, oxygen and sulfur. In another variation, particular heteroaryl groups are polycyclic aromatic rings having from 1 to 12 annular carbon atoms and 1 to 6 annular heteroatoms independently selected from nitrogen, oxygen, and sulfur. A heteroaryl group having more than one ring where at least one ring is non-aromatic may be connected to the parent structure at either an aromatic ring position or at a non-aromatic ring position. In one variation, a heteroaryl group having more than one ring where at least one ring is non-aromatic is connected to the parent structure at an aromatic ring position. A heteroaryl group may be connected to the parent structure at a ring carbon atom or a ring heteroatom.

“Heteroarylene” as used herein refers to the same residues as heteroaryl, but having bivalency.

“Heterocycle”, “heterocyclic”, or “heterocyclyl” as used herein refers to a saturated or an unsaturated non-aromatic cyclic group having a single ring or multiple condensed rings, and having from 1 to 14 annular carbon atoms and from 1 to 6 annular heteroatoms, such as nitrogen, sulfur or oxygen, and the like. A heterocycle comprising more than one ring may be fused, bridged or spiro, or any combination thereof, but excludes heteroaryl. The heterocyclyl group may be optionally substituted independently with one or more substituents described herein. Particular heterocyclyl groups are 3 to 14-membered rings having 1 to 13 annular carbon atoms and 1 to 6 annular heteroatoms independently selected from nitrogen, oxygen and sulfur, 3 to 12-membered rings having 1 to 11 annular carbon atoms and 1 to 6 annular heteroatoms independently selected from nitrogen, oxygen and sulfur, 3 to 10-membered rings having 1 to 9 annular carbon atoms and 1 to 4 annular heteroatoms independently selected from nitrogen, oxygen and sulfur, 3 to 8-membered rings having 1 to 7 annular carbon atoms and 1 to 4 annular heteroatoms independently selected from nitrogen, oxygen and sulfur, or 3 to 6-membered rings having 1 to 5 annular carbon atoms and 1 to 4 annular heteroatoms independently selected from nitrogen, oxygen and sulfur. In one variation, heterocyclyl includes monocyclic 3-, 4-, 5-, 6- or 7-membered rings having from 1 to 2, 1 to 3, 1 to 4, 1 to 5, or 1 to 6 annular carbon atoms and 1 to 2, 1 to 3, or 1 to 4 annular heteroatoms independently selected from nitrogen, oxygen and sulfur. In another variation, heterocyclyl includes polycyclic non-aromatic rings having from 1 to 12 annular carbon atoms and 1 to 6 annular heteroatoms independently selected from nitrogen, oxygen and sulfur.

“Heterocyclylene” as used herein refers to the same residues as heterocyclyl, but having bivalency.

“Halo” or “halogen” refers to elements of the Group 17 series having atomic number 9 to 85. Preferred halo groups include the radicals of fluorine, chlorine, bromine and iodine. Where a residue is substituted with more than one halogen, it may be referred to by using a prefix corresponding to the number of halogen moieties attached, e.g., dihaloaryl, dihaloalkyl, trihaloaryl etc. refer to aryl and alkyl substituted with two (“di”) or three (“tri”) halo groups, which may be but are not necessarily the same halogen; thus 4-chloro-3-fluorophenyl is within the scope of dihaloaryl. An alkyl group in which each hydrogen is replaced with a halo group is referred to as a “perhaloalkyl.” A preferred perhaloalkyl group is trifluoromethyl (—CF₃). Similarly, “perhaloalkoxy” refers to an alkoxy group in which a halogen takes the place of each H in the hydrocarbon making up the alkyl moiety of the alkoxy group. An example of a perhaloalkoxy group is trifluoromethoxy (—OCF₃).

“Carbonyl” refers to the group C═O.

“Thiocarbonyl” refers to the group C═S.

“Oxo” refers to the moiety ═O.

“Optionally substituted” unless otherwise specified means that a group may be unsubstituted or substituted by one or more (e.g., 1, 2, 3, 4 or 5) of the substituents listed for that group in which the substituents may be the same of different. In one embodiment, an optionally substituted group has one substituent. In another embodiment, an optionally substituted group has two substituents. In another embodiment, an optionally substituted group has three substituents. In another embodiment, an optionally substituted group has four substituents. In some embodiments, an optionally substituted group has 1 to 2, 1 to 3, 1 to 4, 1 to 5, 2 to 3, 2 to 4, or 2 to 5 substituents. In one embodiment, an optionally substituted group is unsubstituted.

Unless clearly indicated otherwise, “an individual” as used herein intends a mammal, including but not limited to a primate, human, bovine, horse, feline, canine, or rodent. In one variation, the individual is a human.

As used herein, “treatment” or “treating” is an approach for obtaining beneficial or desired results including clinical results. For purposes of this disclosure, beneficial or desired results include, but are not limited to, one or more of the following: decreasing one more symptoms resulting from the disease, diminishing the extent of the disease, stabilizing the disease (e.g., preventing or delaying the worsening of the disease), preventing or delaying the spread of the disease, delaying the occurrence or recurrence of the disease, delay or slowing the progression of the disease, ameliorating the disease state, providing a remission (whether partial or total) of the disease, decreasing the dose of one or more other medications required to treat the disease, enhancing effect of another medication, delaying the progression of the disease, increasing the quality of life, and/or prolonging survival. The methods of the present disclosure contemplate any one or more of these aspects of treatment.

As used herein, the term “agriculturally effective amount” refers to an amount of a compound or salt thereof sufficient to produce a desired agricultural outcome in a plant. Accordingly, in some embodiments, an agriculturally effective amount may increase protein expression, increase growth, and/or alter the microbial environment adjacent to the plant.

As used herein, the term “effective amount” intends such amount of a compound of the invention which should be effective in a given therapeutic form. As is understood in the art, an effective amount may be in one or more doses, i.e., a single dose or multiple doses may be required to achieve the desired treatment endpoint. An effective amount may be considered in the context of administering one or more therapeutic agents (e.g., a compound, or pharmaceutically acceptable salt thereof), and a single agent may be considered to be given in an effective amount if, in conjunction with one or more other agents, a desirable or beneficial result may be or is achieved. Suitable doses of any of the co-administered compounds may optionally be lowered due to the combined action (e.g., additive or synergistic effects) of the compounds.

A “therapeutically effective amount” refers to an amount of a compound or salt thereof sufficient to produce a desired therapeutic outcome.

As used herein, “unit dosage form” refers to physically discrete units, suitable as unit dosages, each unit containing a predetermined quantity of active ingredient calculated to produce the desired therapeutic effect in association with the required pharmaceutical carrier. Unit dosage forms may contain a single or a combination therapy.

As used herein, by “pharmaceutically acceptable” or “pharmacologically acceptable” is meant a material that is not biologically or otherwise undesirable, e.g., the material may be incorporated into a pharmaceutical composition administered to a patient without causing any significant undesirable biological effects or interacting in a deleterious manner with any of the other components of the composition in which it is contained. Pharmaceutically acceptable carriers or excipients have preferably met the required standards of toxicological and manufacturing testing and/or are included on the Inactive Ingredient Guide prepared by the U.S. Food and Drug administration.

“Pharmaceutically acceptable salts” are those salts which retain at least some of the biological activity of the free (non-salt) compound and which can be administered as drugs or pharmaceuticals to an individual. Such salts, for example, include: (1) acid addition salts, formed with inorganic acids such as hydrochloric acid, hydrobromic acid, sulfuric acid, nitric acid, phosphoric acid, and the like; or formed with organic acids such as acetic acid, oxalic acid, propionic acid, succinic acid, maleic acid, tartaric acid and the like; (2) salts formed when an acidic proton present in the parent compound either is replaced by a metal ion, e.g., an alkali metal ion, an alkaline earth ion, or an aluminum ion; or coordinates with an organic base. Acceptable organic bases include ethanolamine, diethanolamine, triethanolamine and the like. Acceptable inorganic bases include aluminum hydroxide, calcium hydroxide, potassium hydroxide, sodium carbonate, sodium hydroxide, and the like. Pharmaceutically acceptable salts can be prepared in situ in the manufacturing process, or by separately reacting a purified compound of the present disclosure in its free acid or base form with a suitable organic or inorganic base or acid, respectively, and isolating the salt thus formed during subsequent purification.

The term “agriculturally acceptable salt” refers to a salt which retains at least some of the biological activity of the free (non-salt) compound and which can be administered to plants. Such salts, for example, include: (1) acid addition salts, formed with inorganic acids such as hydrochloric acid, hydrobromic acid, sulfuric acid, nitric acid, phosphoric acid, and the like; or formed with organic acids such as acetic acid, oxalic acid, propionic acid, succinic acid, maleic acid, tartaric acid and the like; (2) salts formed when an acidic proton present in the parent compound either is replaced by a metal ion, e.g., an alkali metal ion, an alkaline earth ion, or an aluminum ion; or coordinates with an organic base. Acceptable organic bases include ethanolamine, diethanolamine, triethanolamine and the like. Acceptable inorganic bases include aluminum hydroxide, calcium hydroxide, potassium hydroxide, sodium carbonate, sodium hydroxide, and the like. Agriculturally acceptable salts can be prepared in situ in the manufacturing process, or by separately reacting a purified compound of the present disclosure in its free acid or base form with a suitable organic or inorganic base or acid, respectively, and isolating the salt thus formed during subsequent purification.

The term “excipient” as used herein means an inert or inactive substance that may be used in the production of a drug or pharmaceutical, such as a tablet containing a compound of the present disclosure as an active ingredient. Various substances may be embraced by the term excipient, including without limitation any substance used as a binder, disintegrant, coating, compression/encapsulation aid, cream or lotion, lubricant, solutions for parenteral administration, materials for chewable tablets, sweetener or flavoring, suspending/gelling agent, or wet granulation agent. Binders include, e.g., carbomers, povidone, xanthan gum, etc.; coatings include, e.g., cellulose acetate phthalate, ethylcellulose, gellan gum, maltodextrin, enteric coatings, etc.; compression/encapsulation aids include, e.g., calcium carbonate, dextrose, fructose dc (dc=“directly compressible”), honey dc, lactose (anhydrate or monohydrate; optionally in combination with aspartame, cellulose, or microcrystalline cellulose), starch dc, sucrose, etc.; disintegrants include, e.g., croscarmellose sodium, gellan gum, sodium starch glycolate, etc.; creams or lotions include, e.g., maltodextrin, carrageenans, etc.; lubricants include, e.g., magnesium stearate, stearic acid, sodium stearyl fumarate, etc.; materials for chewable tablets include, e.g., dextrose, fructose dc, lactose (monohydrate, optionally in combination with aspartame or cellulose), etc.; suspending/gelling agents include, e.g., carrageenan, sodium starch glycolate, xanthan gum, etc.; sweeteners include, e.g., aspartame, dextrose, fructose dc, sorbitol, sucrose dc, etc.; and wet granulation agents include, e.g., calcium carbonate, maltodextrin, microcrystalline cellulose, etc.

It is understood that aspects and embodiments described herein as “comprising” include “consisting of” and “consisting essentially of” embodiments.

When a composition is described as “consisting essentially of” the listed components, the composition contains the components expressly listed, and may contain other components which do not substantially affect the disease or condition being treated such as trace impurities. However, the composition either does not contain any other components which do substantially affect the disease or condition being treated other than those components expressly listed; or, if the composition does contain extra components other than those listed which substantially affect the disease or condition being treated, the composition does not contain a sufficient concentration or amount of those extra components to substantially affect the disease or condition being treated. When a method is described as “consisting essentially of” the listed steps, the method contains the steps listed, and may contain other steps that do not substantially affect the disease or condition being treated, but the method does not contain any other steps which substantially affect the disease or condition being treated other than those steps expressly listed.

When a moiety is indicated as substituted by “at least one” substituent, this also encompasses the disclosure of exactly one substituent.

### Compounds

In a first aspect, provided is a compound of formula (I)

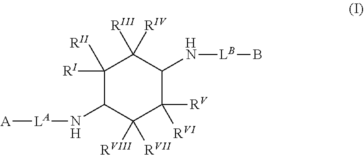

or a pharmaceutically acceptable salt thereof,
wherein:
R^{I}, R^{II}, R^{III}, R^{IV}, R^{V}, R^{VI}, R^{VII}, and R^{VIII}, independently from each other, are selected from the group consisting of hydrogen, C₁-C₆ alkyl, C₁-C₆ haloalkyl, —C(O)OH, —C(O)O(C₁-C₆ alkyl), —C(O)O(C₁-C₆ haloalkyl), and halogen;
or, one of R^{I}, R^{II}, R^{III}, R^{IV}, R^{V}, R^{VI}, R^{VII}, and R^{VIII}, and another one of R^{I}, R^{II}, R^{III}, R^{IV}, R^{V}, R^{VI}, R^{VII}, and R^{VIII}, are taken together to form a C₁-C₆ alkylene moiety;
or, two geminal substituents selected from the group consisting of R^{I}, R^{II}, R^{III}, R^{IV}, R^{V}, R^{VI}, R^{VII}, and R^{VIII} are taken together to form an oxo group;
L^{A} is selected from the group consisting of

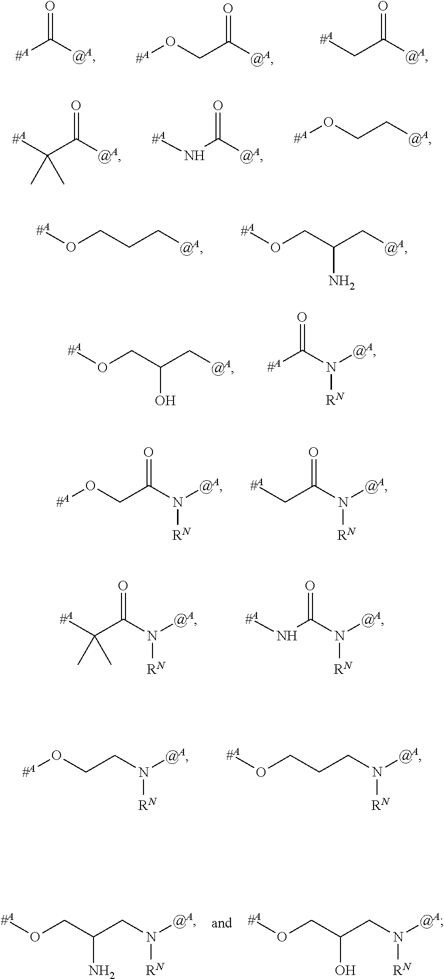

wherein #^{A} represents the attachment point to A and @^{A} represents the attachment point to the remainder of the molecule;
L^{B} is selected from the group consisting of

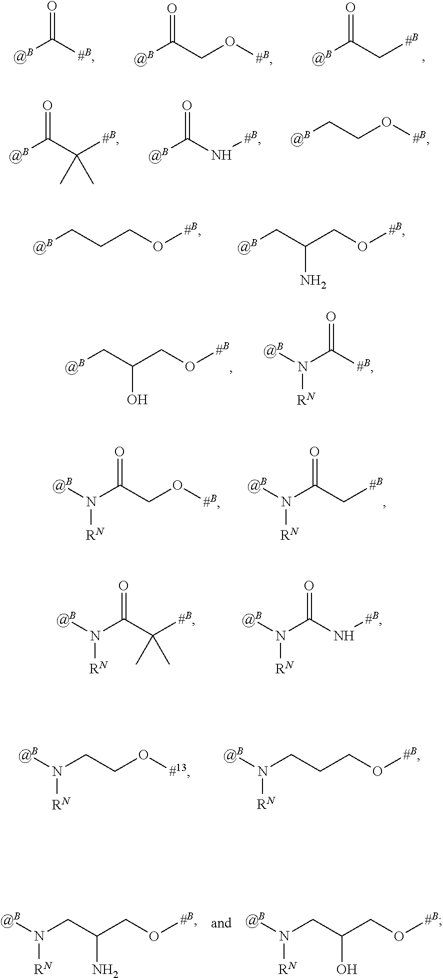

wherein #^{B} represents the attachment point to B and @^{B} represents the attachment point to the remainder of the molecule;
R^{N}, independently at each occurrence, is selected from the group consisting of hydrogen, C₁-C₆ alkyl, and C₁-C₆ haloalkyl,
A is a substituent of formula (A-I)

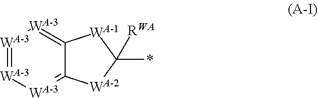

wherein * represents the attachment point to the remainder of the molecule;
   W^{A-1} is selected from the group consisting of —C(R^{WA-1-1}R^{WA-1-2}), —N(R^{WA-1-2})—, —C(R^{WA-1-1}R^{WA-1-1})N(R^{WA-1-2})—, —N(R^{WA-1-1})C(R^{WA-1-1}R^{WA-1-2})—, —C(R^{WA-1-1})═N—, —N═C(R^{WA-1-1})—, —O—, —C(R^{WA-1-1}R^{WA-1-1})O—, —OC(R^{WA-1-1}R^{WA-1-2})—, —S—, —C(R^{WA-1-1}R^{WA-1-1})S—, —SC(R^{WA-1-1}R^{WA-1-2})—, —C(R^{WA-1-1}R^{WA-1-1})C(R^{WA-1-1}R^{WA-1-2})—, and —CR^{WA-1-1}═CR^{WA-1-1}—,
      wherein R^{WA-1-1} is H or R^{A}, and R^{WA-1-2} is H or R^{A};
   W^{A-2} is selected from the group consisting of —C(R^{WA-2-1}R^{WA-2-2})—, —N(R^{WA-2-2})—, —C(R^{WA-2-1}R^{WA-2-1})N(R^{WA-2-2})—, —N(R^{WA-2-1})C(R^{WA-2-1}R^{WA-2-2})—, —C(R^{WA-2-1})═N—, —N═C(R^{WA-2-1})—, —O—, —C(R^{WA-2-1}R^{WA-2-1})O—, —OC(R^{WA-2-1}R^{WA-2-2})—, —S—, —C(R^{WA-2-1}R^{WA-2-1})S—, —SC(R^{WA-2-1}R^{WA-2-2})—, —C(R^{WA-2-1}R^{WA-2-1})C(R^{WA-2-1}R^{WA-2-2})—, and —CR^{WA-2-1}═CR^{WA-2-1}—,
      wherein R^{WA-2-1} is H or R^{A}, and R^{WA-2-2} is H or R^{A};
   W^{A-3}, independently at each occurrence, is CR^{WA-3} or N, wherein R^{WA-3} is H or R^{A}.
   R^{WA} is hydrogen or R^{A}, or R^{WA} and R^{WA-1-2} are taken together to form a double bond between the carbon atom bearing R^{WA} and the atom bearing R^{WA-1-2}, or R^{WA} and R^{WA-2-2} are taken together to form a double bond between the carbon atom bearing R^{WA} and the atom bearing R^{WA-2-2};
   C₆-C₁₄ aryl optionally substituted with 1, 2, 3, 4, 5, 6, 7, 8, or 9 R^{A} substituents; and
   5-14 membered heteroaryl optionally substituted with 1, 2, 3, 4, 5, 6, 7, 8, or 9 R^{A} substituents;
      R^{A}, independently at each occurrence, is selected from the group consisting of halogen, NO₂, C₁-C₆ alkyl, C₂-C₆ alkenyl, C₂-C₆ alkynyl, C₁-C₆ haloalkyl, OH, O(C₁-C₆ alkyl), O(C₁-C₆ haloalkyl), SH, S(C₁-C₆ alkyl), S(C₁-C₆ haloalkyl), NH₂, NH(C₁-C₆ alkyl), NH(C₁-C₆ haloalkyl), N(C₁-C₆ alkyl)₂, N(C₁-C₆ haloalkyl)₂, NR^{a}R^{b}, CN, C(O)OH, C(O)O(C₁-C₆ alkyl), C(O)O(C₁-C₆ haloalkyl), C(O)NH₂, C(O)NH(C₁-C₆ alkyl), C(O)NH(C₁-C₆ haloalkyl), C(O)N(C₁-C₆ alkyl)₂, C(O)N(C₁-C₆ haloalkyl)₂, C(O)NR^{a}R^{b}, S(O)₂OH, S(O)₂O(C₁-C₆ alkyl), S(O)₂O(C₁-C₆ haloalkyl), S(O)₂NH₂, S(O)₂NH(C₁-C₆ alkyl), S(O)₂NH(C₁-C₆ haloalkyl), S(O)₂N(C₁-C₆ alkyl)₂, S(O)₂N(C₁-C₆ haloalkyl)₂, S(O)₂NR^{a}R^{b}, OC(O)H, OC(O)(C₁-C₆ alkyl), OC(O)(C₁-C₆ haloalkyl), N(H)C(O)H, N(H)C(O)(C₁-C₆ alkyl), N(H)C(O)(C₁-C₆ haloalkyl), N(C₁-C₆ alkyl)C(O)H, N(C₁-C₆ alkyl)C(O)(C₁-C₆ alkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ alkyl), OS(O)₂(C₁-C₆ haloalkyl), N(H)S(O)₂(C₁-C₆ alkyl), N(H)S(O)₂(C₁-C₆ haloalkyl), N(C₁-C₆ alkyl)S(O)₂(C₁-C₆ alkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl); wherein R^{a} and R^{b} are taken together with the nitrogen atom to which they are attached to form a 3-10 membered heterocycle;
         and
B is selected from the group consisting of:
   a substituent of formula (B-I)

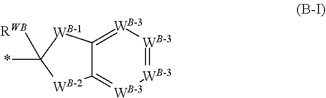

wherein * represents the attachment point to the remainder of the molecule;
   W^{B-1} is selected from the group consisting of —C(R^{WB-1-1}R^{WB-1-2})—, —N(R^{WB-1-2})—, —C(R^{WB-1-1}R^{WB-1-2})N(R^{WB-1-2})—, —N(R^{WB-1-1})C(R^{WB-1-1}R^{WB-1-2})—, —C(R^{WB-1-1})═N—, —N═C(R^{WB-1-1})—, —O—, —C(R^{WB-1-1}R^{WB-1-1})O—, —OC(R^{WB-1-1}R^{WB-1-2})—, —S—, —C(R^{WB-1-1}R^{WB-1-1})S—, —SC(R^{WB-1-1}R^{WB-1-2})—, —C(R^{WB-1-1}R^{WB-1-1})C(R^{WB-1-1}R^{WB-1-2})—, and —CR^{WB-1-1}═CR^{WB-1-1}—,
      wherein R^{WB-1-1} is H or R^{B}, and R^{WB-1-2} is H or R^{B};
   W^{B-2} is selected from the group consisting of —C(R^{WB-2-1}R^{WB-2-2}), —N(R^{WB-2-2})—, —C(R^{WB-2-1}R^{WB-2-1})N(R^{WB-2-2})—, —N(R^{WB-2-1})C(R^{WB-2-1}R^{WB-2-2})—, —C(R^{WB-2-1})═N—, —N═C(R^{WB-2-1})—, —O—, —C(R^{WB-2-1}R^{WB-2-1})O—, —OC(R^{WB-2-1}R^{WB-2-2})—, —S—, —C(R^{WB-2-1}R^{WB-2-1})S—, —SC(R^{WB-2-1}R^{WB-2-2})—, —C(R^{WB-2-1}R^{WB-2-1})C(R^{WB-2-1}R^{WB-2-2})—, and —CR^{WB-2-1}═CR^{WB-2-1}—,
      wherein R^{WB-2-1} is H or R^{B}, and R^{WB-2-2} is H or R^{B};
   W^{B-3}, independently at each occurrence, is CR^{WB-3} or N, wherein R^{WB-3} is H or R^{B}.
   R^{WB} is hydrogen or R^{B}, or R^{WB} and R^{WB-1-2} are taken together to form a double bond between the carbon atom bearing R^{WB} and the atom bearing R^{WB-1-2}, or R^{WB} and R^{WB-2-2} are taken together to form a double bond between the carbon atom bearing R^{WB} and the atom bearing R^{WB-2-2};
C₆-C₁₄ aryl optionally substituted with 1, 2, 3, 4, 5, 6, 7, 8, or 9 R^{B} substituents; and
5-14 membered heteroaryl optionally substituted with 1, 2, 3, 4, 5, 6, 7, 8, or 9 R^{B} substituents;
   R^{B}, independently at each occurrence, is selected from the group consisting of halogen, NO₂, C₁-C₆ alkyl, C₂-C₆ alkenyl, C₂-C₆ alkynyl, C₁-C₆ haloalkyl, OH, O(C₁-C₆ alkyl), O(C₁-C₆ haloalkyl), SH, S(C₁-C₆ alkyl), S(C₁-C₆ haloalkyl), NH₂, NH(C₁-C₆ alkyl), NH(C₁-C₆ haloalkyl), N(C₁-C₆ alkyl)₂, N(C₁-C₆ haloalkyl)₂, NR^{a}R^{b}, CN, C(O)OH, C(O)O(C₁-C₆ alkyl), C(O)O(C₁-C₆ haloalkyl), C(O)NH₂, C(O)NH(C₁-C₆ alkyl), C(O)NH(C₁-C₆ haloalkyl), C(O)N(C₁-C₆ alkyl)₂, C(O)N(C₁-C₆ haloalkyl)₂, C(O)NR^{a}R^{b}, S(O)₂OH, S(O)₂O(C₁-C₆ alkyl), S(O)₂O(C₁-C₆ haloalkyl), S(O)₂NH₂, S(O)₂NH(C₁-C₆ alkyl), S(O)₂NH(C₁-C₆ haloalkyl), S(O)₂N(C₁-C₆ alkyl)₂, S(O)₂N(C₁-C₆ haloalkyl)₂, S(O)₂NR^{a}R^{b}, OC(O)H, OC(O)(C₁-C₆ alkyl), OC(O)(C₁-C₆ haloalkyl), N(H)C(O)H, N(H)C(O)(C₁-C₆ alkyl), N(H)C(O)(C₁-C₆ haloalkyl), N(C₁-C₆ alkyl)C(O)H, N(C₁-C₆ alkyl)C(O)(C₁-C₆ alkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ alkyl), OS(O)₂(C₁-C₆ haloalkyl), N(H)S(O)₂(C₁-C₆ alkyl), N(H)S(O)₂(C₁-C₆ haloalkyl), N(C₁-C₆ alkyl)S(O)₂(C₁-C₆ alkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl); wherein R^{a} and R^{b} are taken together with the nitrogen atom to which they are attached to form a 3-10 membered heterocycle.

In some embodiments of the compounds of formula (I), or the salts thereof, R^{I}, R^{II}, R^{III}, R^{IV}, R^{V}, R^{VI}, R^{VII}, and R^{VIII} are each hydrogen. In some embodiments, R^{IV} and R^{VIII} are taken together to form a C₁-C₆ alkylene moiety. In some embodiments, R^{IV} and R^{VIII} are taken together to form a moiety selected from methylene, ethylene, and propylene. In some embodiments, R^{IV} and R^{VIII} are taken together to form a methylene moiety. In some embodiments, R^{IV} and R^{VIII} are taken together to form an ethylene moiety. In some embodiments, R^{I}, R^{II}, R^{III}, R^{V}, R^{VI}, and R^{VII} are each hydrogen, and R^{IV} and R^{VIII} are taken together to form a C₁-C₆ alkylene moiety. In some embodiments, R^{I}, R^{II}, R^{III}, R^{V}, R^{VI}, and R^{VII} are each hydrogen, and R^{IV} and R^{VIII} are taken together to form a moiety selected from methylene, ethylene, and propylene. In some embodiments, R^{I}, R^{II}, R^{III}, R^{V}, R^{VI}, and R^{VII} are each hydrogen, and R^{IV} and R^{VIII} are taken together to form a methylene moiety. In some embodiments, R^{I}, R^{II}, R^{III}, R^{V}, R^{VI}, and R^{VII} are each hydrogen, and R^{IV} and R^{VIII} are taken together to form an ethylene moiety.

In some embodiments of the compounds of formula (I), or the salts thereof, L^{A} is selected from the group consisting of

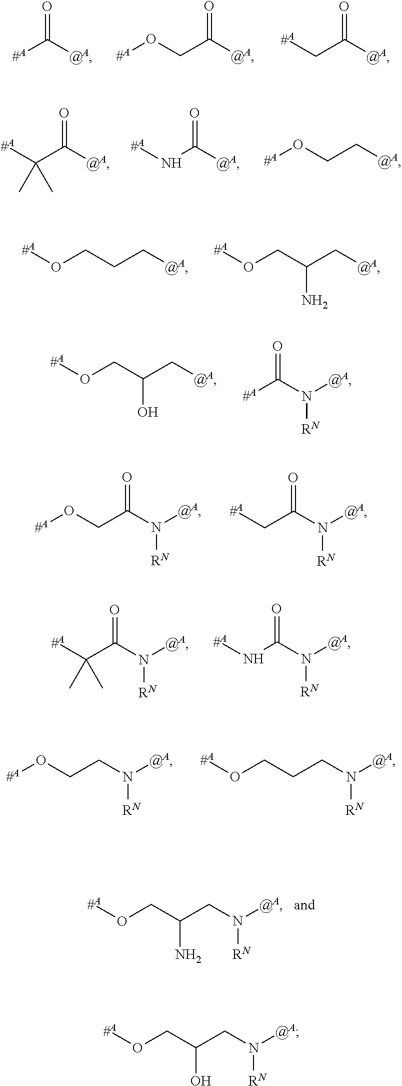

wherein #^{A} represents the attachment point to A and @^{A} represents the attachment point to the remainder of the molecule. In some embodiments, L^{A} is selected from the group consisting of

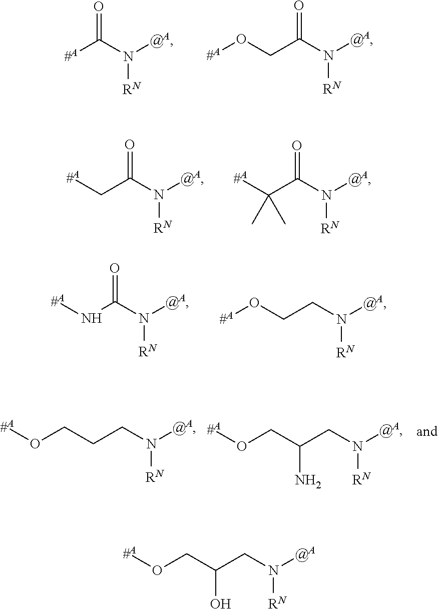

In some embodiments, L^{A} is selected from the group consisting of

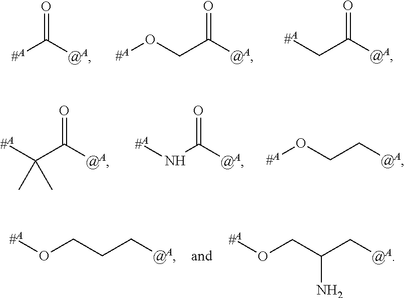

In some embodiments, L^{A} is

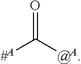

In some embodiments, L^{A} is

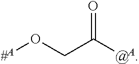

In some embodiments, L^{A} is

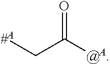

In some embodiments, L^{A} is

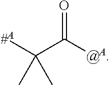

In some embodiments, L^{A} is

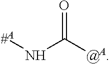

In some embodiments, L^{A} is

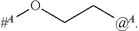

In some embodiments, L^{A} is

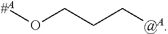

In some embodiments, L^{A} is

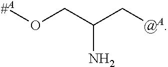

In some embodiments, L^{A} is

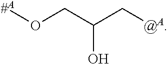

In some embodiments, L^{A} is

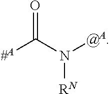

In some embodiments, L^{A} is

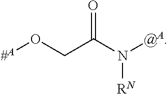

In some embodiments, L^{A} is

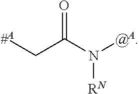

In some embodiments, L^{A} is

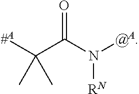

In some embodiments, L^{A} is

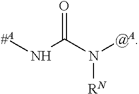

In some embodiments, L^{A} is

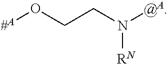

In some embodiments, L^{A} is

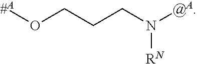

In some embodiments, L^{A} is

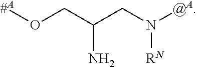

In some embodiments, L^{A} is

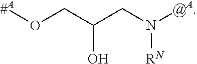

In some embodiments of the compounds of formula (I), or the salts thereof, L^{B} is selected from the group consisting of

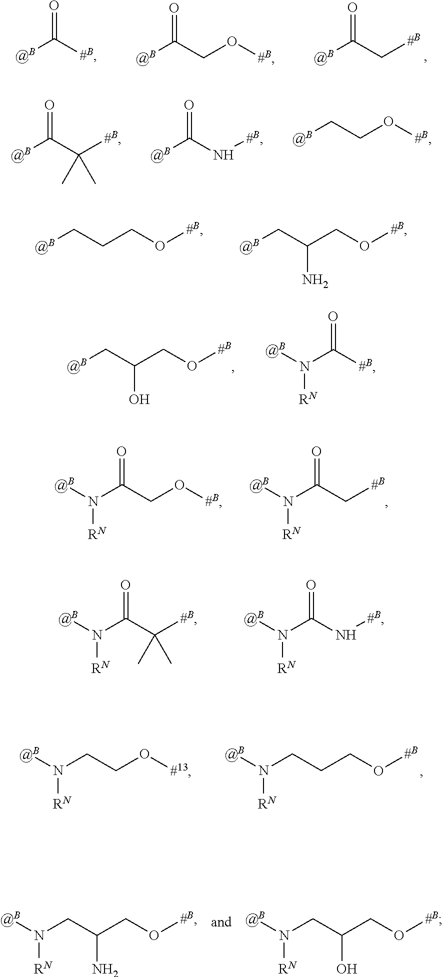

wherein #^{B} represents the attachment point to B and @^{B} represents the attachment point to the remainder of the molecule. In some embodiments, L^{B} is selected from the group consisting of

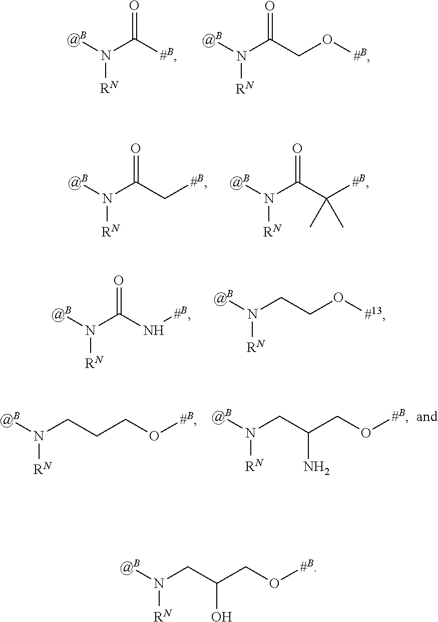

In some embodiments, L^{B} is selected from the group consisting of

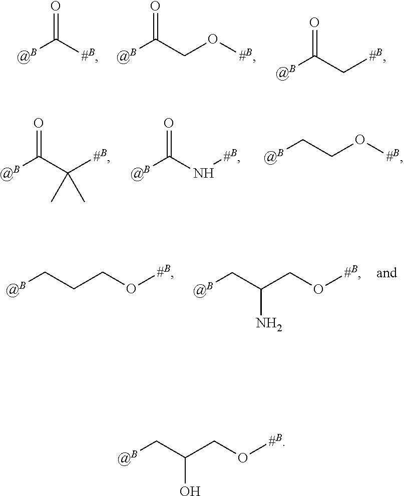

In some embodiments, L^{B} is

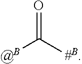

In some embodiments, L^{B} is

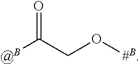

In some embodiments, L^{B} is

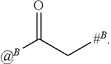

In some embodiments, L^{B} is

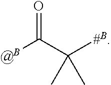

In one embodiments, L^{B} is

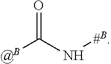

In some embodiments, L^{B} is

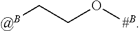

In some embodiments, L^{B} is

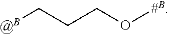

In some embodiments, L^{B} is

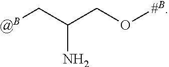

In some embodiments, L^{B} is

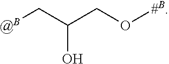

In some embodiments, L^{B} is

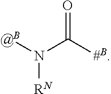

In some embodiments, L^{B} is

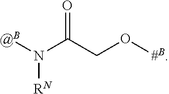

In some embodiments, L^{B} is

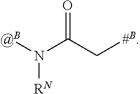

In some embodiments, L^{B} is

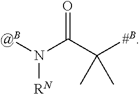

In some embodiments, L^{B} is

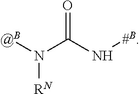

In some embodiments, L^{B} is

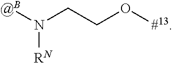

In some embodiments, L^{B} is

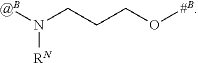

In some embodiments, L^{B} is

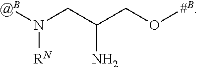

In some embodiments, L^{B} is

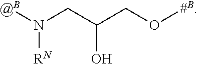

In some embodiments of the compounds of formula (I), or the salts thereof, A is a substituent of formula (A-I)

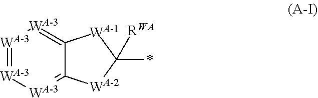

wherein * represents the attachment point to the remainder of the molecule;
W^{A-1} is selected from the group consisting of —C(R^{WA-1-1}R^{WA-1-2}), —N(R^{WA-1-2})—, —C(R^{WA-1-1}R^{WA-1-1})N(R^{WA-1-2})—, —N(R^{WA-1-1})C(R^{WA-1-1}R^{WA-1-2})—, —C(R^{WA-1-1})═N—, —N═C(R^{WA-1-1})—, —O—, —C(R^{WA-1-1}R^{WA-1-1})O—, —OC(R^{WA-1-1}R^{WA-1-2})—, —S—, —C(R^{WA-1-1}R^{WA-1-1})S—, —SC(R^{WA-1-1}R^{WA-1-2})—, C(R^{WA-1-1}R^{WA-1-1})C(R^{WA-1-1}R^{WA-1-2})—, and —CR^{WA-1-1}═CR^{WA-1-1}—,

wherein R^{WA-1-1} is H or R^{A}, and R^{WA-1-2} is H or R^{A};

W^{A-2} is selected from the group consisting of —C(R^{WA-2-1}R^{WA-2-2})—, —N(R^{WA-2-2})—, —C(R^{WA-2-1}R^{WA-2-1})N(R^{WA-2-2})—, —N(R^{WA-2-1})C(R^{WA-2-1}R^{WA-2-2})—, —C(R^{WA-2-1})═N—, —N═C(R^{WA-2-1})—, —O—, —C(R^{WA-2-1}R^{WA-2-1})O—, —OC(R^{WA-2-1}R^{WA-2-2})—, —S—, —C(R^{WA-2-1}R^{WA-2-1})S—, —SC(R^{WA-2-1}R^{WA-2-2})—, —C(R^{WA-2-1}R^{WA-2-1})C(R^{WA-2-1}R^{WA-2-2})—, and —CR^{WA-2-1}═CR^{WA-2-1}—,

wherein R^{WA-2-1} is H or R^{A}, and R^{WA-2-2} is H or R^{A};

W^{A-3}, independently at each occurrence, is CR^{WA-3} or N, wherein R^{WA-3} is H or R^{A}; R^{WA} is hydrogen or R^{A}, or R^{WA} and R^{WA-1-2} are taken together to form a double bond between the carbon atom bearing R^{WA} and the atom bearing R^{WA-1-2}, or R^{WA} and R^{WA-2-2} are taken together to form a double bond between the carbon atom bearing R^{WA} and the atom bearing R^{WA-2-2}.

In some embodiments, (A-I) is selected from the group consisting of

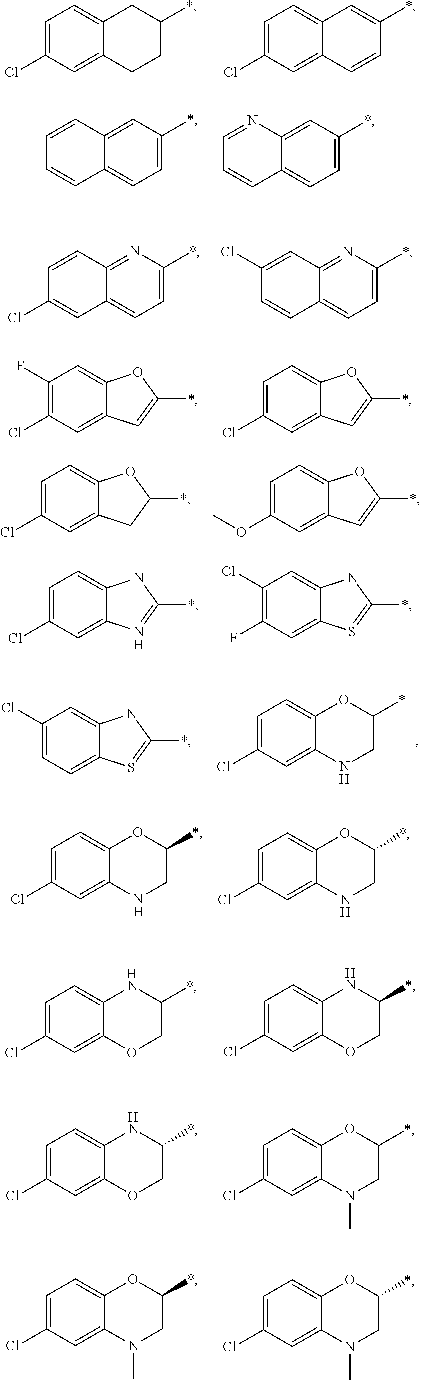

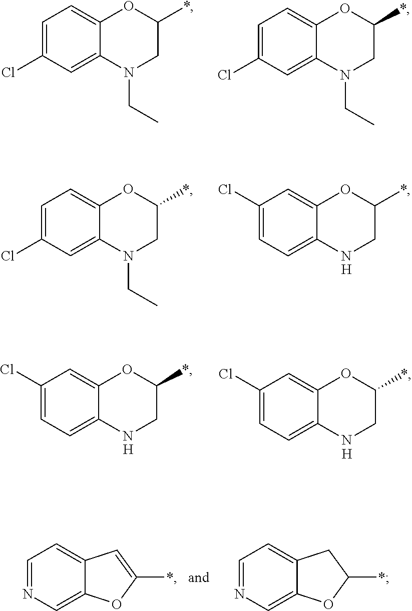

wherein * represents the attachment point to the remainder of the molecule. In some embodiments (A-I) is selected from the group consisting of

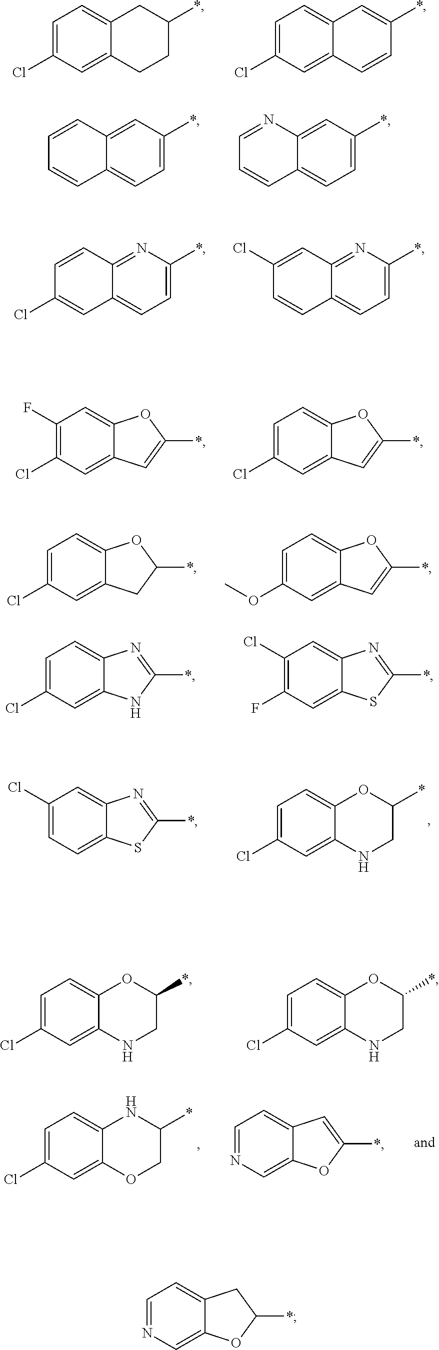

wherein * represents the attachment point to the remainder of the molecule. In some embodiments, (A-I) is

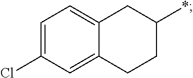

wherein * represents the attachment point to the remainder of the molecule. In some embodiments, (A-I) is

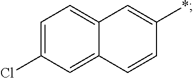

wherein * represents the attachment point to the remainder of the molecule. In some embodiments, (A-I) is

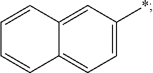

wherein * represents the attachment point to the remainder of the molecule. In some embodiments, (A-I) is

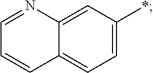

wherein * represents the attachment point to the remainder of the molecule. In some embodiments, (A-I) is

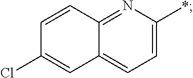

wherein * represents the attachment point to the remainder of the molecule. In some embodiments, (A-I) is

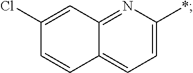

wherein * represents the attachment point to the remainder of the molecule. In some embodiments, (A-I) is

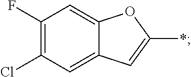

wherein * represents the attachment point to the remainder of the molecule. In some embodiments, (A-I) is

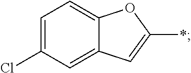

wherein * represents the attachment point to the remainder of the molecule. In some embodiments, (A-I) is

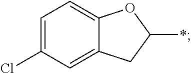

wherein * represents the attachment point to the remainder of the molecule. In some embodiments, (A-I) is

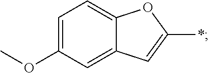

wherein * represents the attachment point to the remainder of the molecule. In some embodiments, (A-I) is

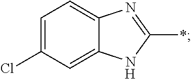

wherein * represents the attachment point to the remainder of the molecule. In some embodiments, (A-I) is

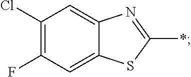

wherein * represents the attachment point to the remainder of the molecule. In some embodiments, (A-I) is

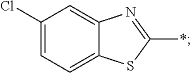

wherein * represents the attachment point to the remainder of the molecule. In some embodiments, (A-I) is

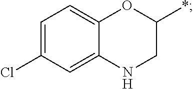

wherein * represents the attachment point to the remainder of the molecule. In some embodiments, (A-I) is

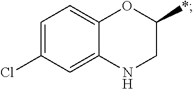

wherein * represents the attachment point to the remainder of the molecule. In some embodiments, (A-I) is

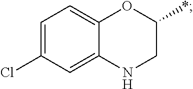

wherein * represents the attachment point to the remainder of the molecule. In some embodiments, (A-I) is

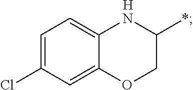

wherein * represents the attachment point to the remainder of the molecule. In some embodiments, (A-I) is

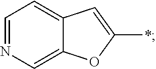

wherein * represents the attachment point to the remainder of the molecule. In some embodiments, (A-I) is

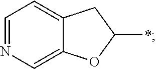

wherein * represents the attachment point to the remainder of the molecule.

In some embodiments of the compounds of formula (I), or the salts thereof, A is C₆-C₁₄ aryl optionally substituted with 1, 2, 3, 4, 5, 6, 7, 8, or 9 R^{A} substituents. In some embodiments, A is selected from the group consisting of

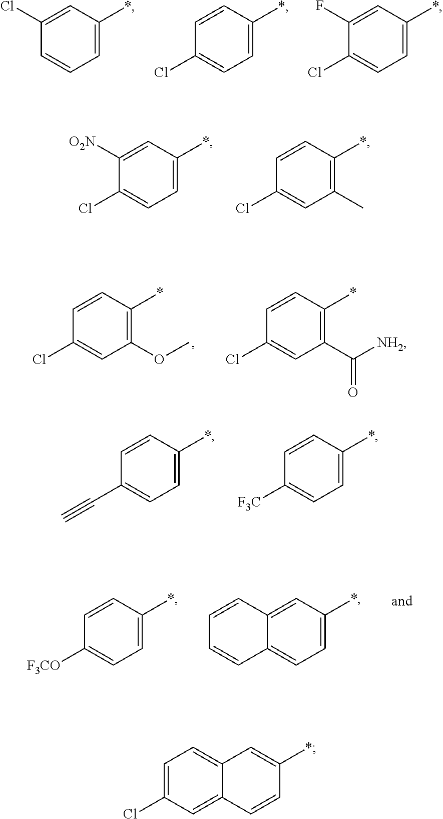

wherein * represents the attachment point to the remainder of the molecule. In some embodiments, A is selected from the group consisting of

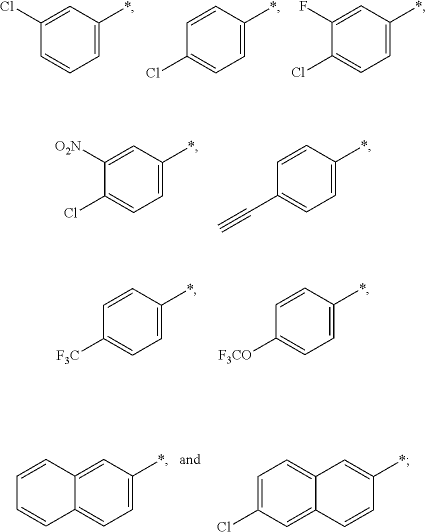

wherein * represents the attachment point to the remainder of the molecule. In some embodiments, A is

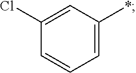

wherein * represents the attachment point to the remainder of the molecule. In some embodiments, A is

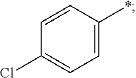

wherein * represents the attachment point to the remainder of the molecule. In some embodiments, A is

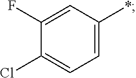

wherein * represents the attachment point to the remainder of the molecule. In some embodiments, A is

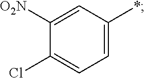

wherein * represents the attachment point to the remainder of the molecule. In some embodiments, A is

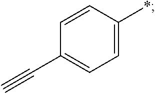

wherein * represents the attachment point to the remainder of the molecule. In some embodiments, A is

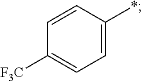

wherein * represents the attachment point to the remainder of the molecule. In some embodiments, A is

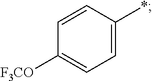

wherein * represents the attachment point to the remainder of the molecule. In some embodiments, A is

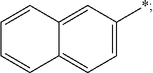

wherein * represents the attachment point to the remainder of the molecule. In some embodiments, A is

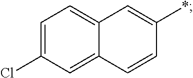

wherein * represents the attachment point to the remainder of the molecule.

In some embodiments of the compounds of formula (I), or the salts thereof, A is 5-14 membered heteroaryl optionally substituted with 1, 2, 3, 4, 5, 6, 7, 8, or 9 R^{A} substituents. In some embodiments, A is selected from the group consisting of

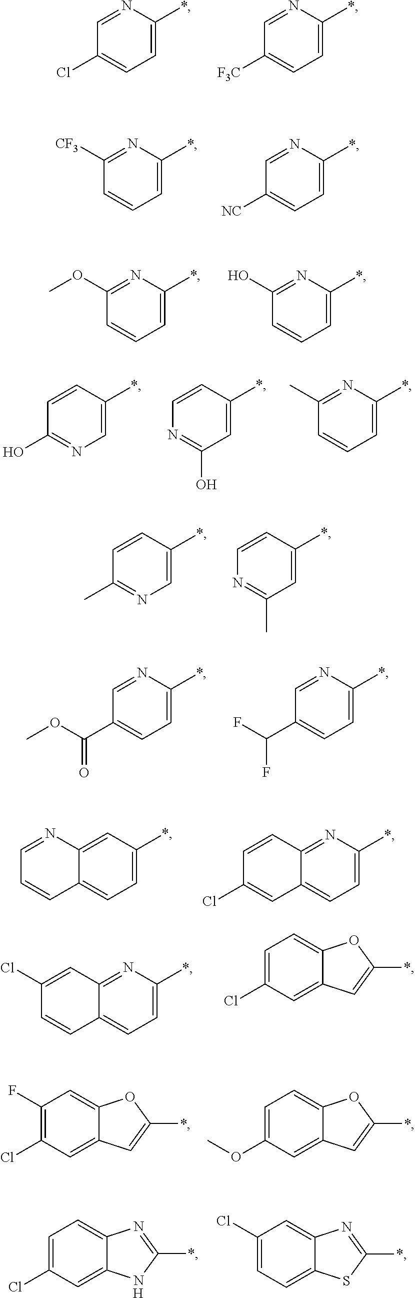

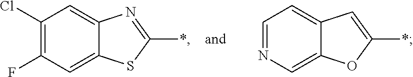

wherein * represents the attachment point to the remainder of the molecule. In some embodiments, A is selected from the group consisting of

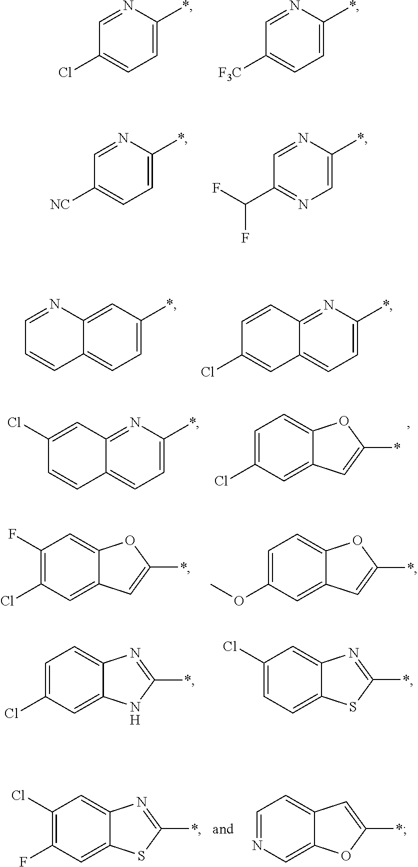

wherein * represents the attachment point to the remainder of the molecule. In some embodiments, A is

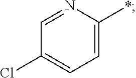

wherein * represents the attachment point to the remainder of the molecule. In some embodiments, A is

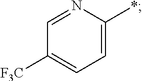

wherein * represents the attachment point to the remainder of the molecule. In some embodiments, A is

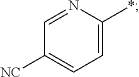

wherein * represents the attachment point to the remainder of the molecule. In some embodiments, A is

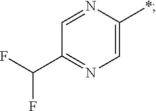

wherein * represents the attachment point to the remainder of the molecule. In some embodiments, A is

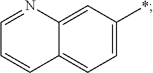

wherein * represents the attachment point to the remainder of the molecule. In some embodiments, A is

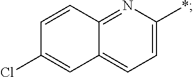

wherein * represents the attachment point to the remainder of the molecule. In some embodiments, A is

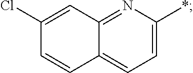

wherein * represents the attachment point to the remainder of the molecule. In some embodiments, A is

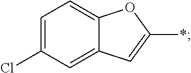

wherein * represents the attachment point to the remainder of the molecule. In some embodiments, A is

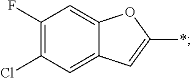

wherein * represents the attachment point to the remainder of the molecule. In some embodiments, A is

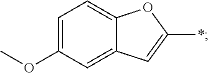

wherein * represents the attachment point to the remainder of the molecule. In some embodiments, A is

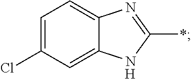

wherein * represents the attachment point to the remainder of the molecule. In some embodiments, A is

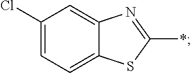

wherein * represents the attachment point to the remainder of the molecule. In some embodiments, A is

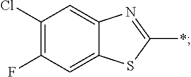

wherein * represents the attachment point to the remainder of the molecule. In some embodiments, A is

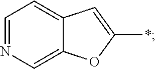

wherein * represents the attachment point to the remainder of the molecule.

In some embodiments of the compounds of formula (I), B is a substituent of formula (B-I)

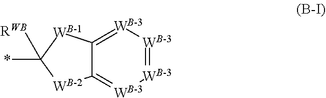

wherein * represents the attachment point to the remainder of the molecule;
W^{B-1} is selected from the group consisting of —C(R^{WB-1-1}R^{WB-1-2})—, —N(R^{WB-1-2})—, —C(R^{WB-1-1}R^{WB-1-2})N(R^{WB-1-2})—, —N(R^{WB-1-1})C(R^{WB-1-1}R^{WB-1-2})—, —C(R^{WB-1-1})═N—, —N═C(R^{WB-1-1})—, —O—, —C(R^{WB-1-1}R^{WB-1-1})O—, —OC(R^{WB-1-1}R^{WB-1-2})—, —S—, —C(R^{WB-1-1}R^{WB-1-1})S—, —SC(R^{WB-1-1}R^{WB-1-2})—, —C(R^{WB-1-1}R^{WB-1-1})C(R^{WB-1-1}R^{WB-1-2})—, and —CR^{WB-1-1}═CR^{WB-1-1}—,

wherein R^{WB-1-1} is H or R^{B}, and R^{WB-1-2} is H or R^{B};

W^{B-2} is selected from the group consisting of —C(R^{WB-2-1}R^{WB-2-2})—, —N(R^{WB-2-2})—, —C(R^{WB-2-1}R^{WB-2-1})N(R^{WB-2-2})—, —N(R^{WB-2-1})C(R^{WB-2-1}R^{WB-2-2})—, —C(R^{WB-2-1})═N—, —N═C(R^{WB-2-1})—, —O—, —C(R^{WB-2-1}R^{WB-2-1})O—, —OC(R^{WB-2-1}R^{WB-2-2})—, —S—, —C(R^{WB-2-1}R^{WB-2-1})S—, —SC(R^{WB-2-1}R^{WB-2-2})—, —C(R^{WB-2-1}R^{WB-2-1})C(R^{WB-2-1}R^{WB-2-2})—, and —CR^{WB-2-1}═CR^{WB-2-1}—,

wherein R^{WB-2-1} is H or R^{B}, and R^{WB-2-2} is H or R^{B}.

W^{B-3}, independently at each occurrence, is CR^{WB-3} or N, wherein R^{WB-3} is H or R^{B};
R^{WB} is hydrogen or R^{B}, or R^{WB} and R^{WB-1-2} are taken together to form a double bond between the carbon atom bearing R^{WB} and the atom bearing R^{WB-1-2}, or R^{WB} and R^{WB-2-2} are taken together to form a double bond between the carbon atom bearing R^{WB} and the atom bearing R^{WB-2-2}.

In some embodiments, (B-I) is selected from the group consisting of

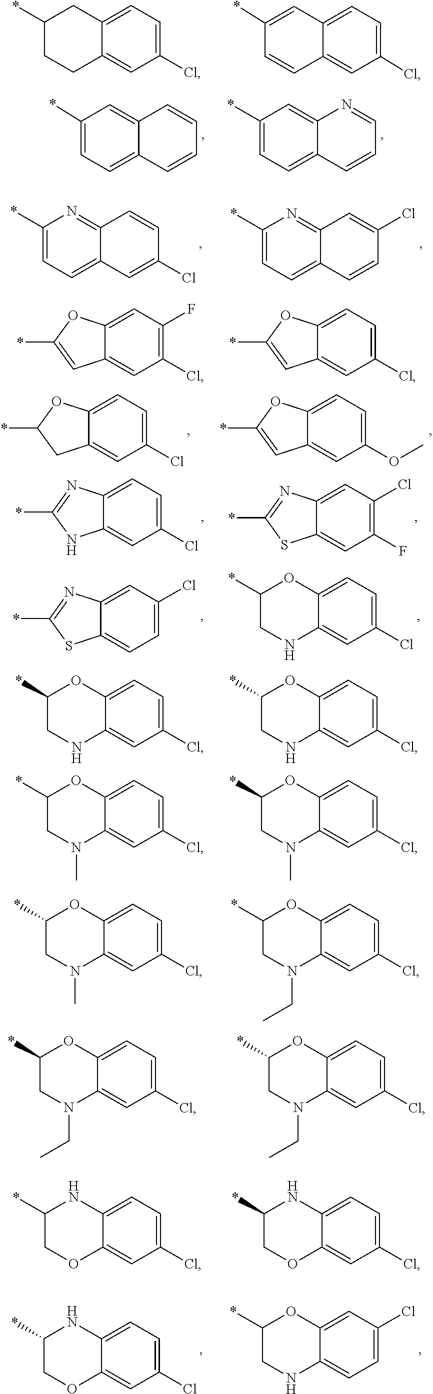

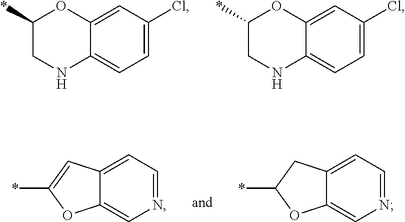

wherein * represents the attachment point to the remainder of the molecule. In some embodiments, (B-I) is selected from the group consisting of

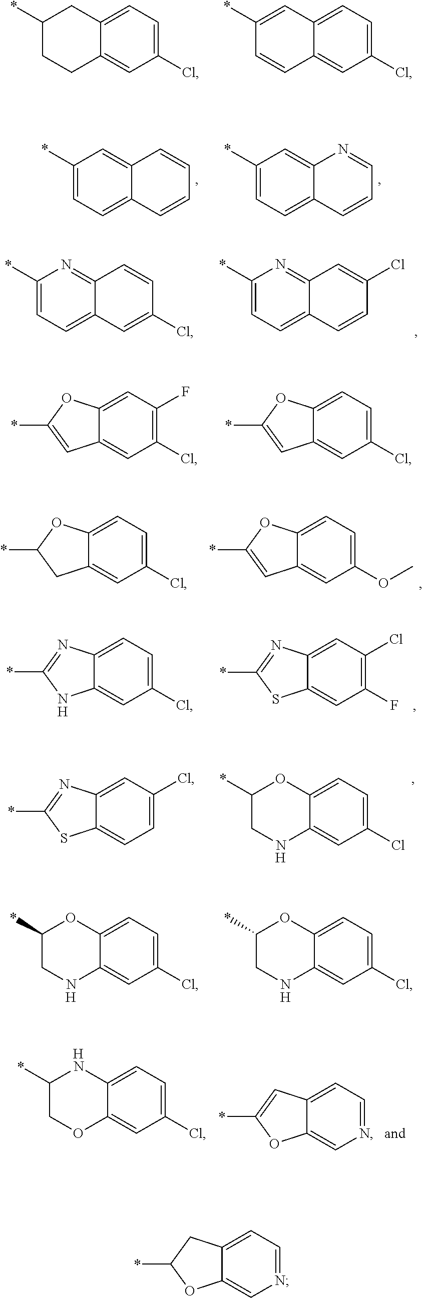

wherein * represents the attachment point to the remainder of the molecule. In some embodiments, (B-I) is

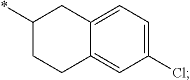 [SMILES: *C1CCc2cc(Cl)ccc2C1]

wherein * represents the attachment point to the remainder of the molecule. In some embodiments, (B-I) is

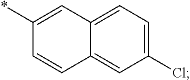 [SMILES: *c1ccc2cc(Cl)ccc2c1]

wherein * represents the attachment point to the remainder of the molecule. In some embodiments, (B-I) is

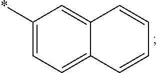 [SMILES: *c1ccc2ccccc2c1]

wherein * represents the attachment point to the remainder of the molecule. In some embodiments, (B-I) is

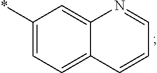 [SMILES: *c1ccc2cccnc2c1]

wherein * represents the attachment point to the remainder of the molecule. In some embodiments, (B-I) is

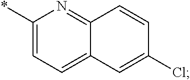 [SMILES: *c1ccc2cc(Cl)ccc2n1]

wherein * represents the attachment point to the remainder of the molecule. In some embodiments, (B-I) is

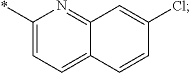 [SMILES: *c1ccc2ccc(Cl)cc2n1]

wherein * represents the attachment point to the remainder of the molecule. In some embodiments, (B-I) is

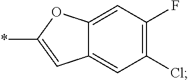 [SMILES: *c1cc2cc(Cl)c(F)cc2o1]

wherein * represents the attachment point to the remainder of the molecule. In some embodiments, (B-I) is

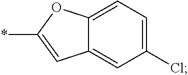 [SMILES: *c1cc2cc(Cl)ccc2o1]

wherein * represents the attachment point to the remainder of the molecule. In some embodiments, (B-I) is

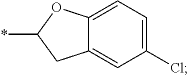 [SMILES: *C1Cc2cc(Cl)ccc2O1]

wherein * represents the attachment point to the remainder of the molecule. In some embodiments, (B-I) is

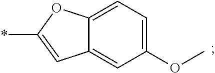 [SMILES: *c1cc2cc(OC)ccc2o1]

wherein * represents the attachment point to the remainder of the molecule. In some embodiments, (B-I) is

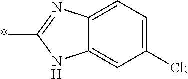

wherein * represents the attachment point to the remainder of the molecule. In some embodiments, (B-I) is

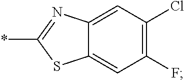 [SMILES: *c1nc2cc(Cl)c(F)cc2s1]

wherein * represents the attachment point to the remainder of the molecule. In some embodiments, (B-I) is

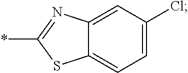 [SMILES: *c1nc2cc(Cl)ccc2s1]

wherein * represents the attachment point to the remainder of the molecule. In some embodiments, (B-I) is

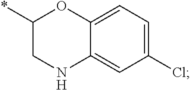

wherein * represents the attachment point to the remainder of the molecule. In some embodiments, (B-I) is

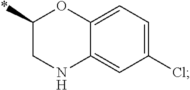

wherein * represents the attachment point to the remainder of the molecule. In some embodiments, (B-I) is

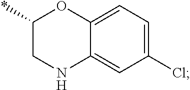

wherein * represents the attachment point to the remainder of the molecule. In some embodiments, (B-I) is

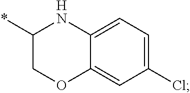

wherein * represents the attachment point to the remainder of the molecule. In some embodiments, (B-I) is

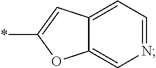 [SMILES: *c1cc2ccncc2o1]

wherein * represents the attachment point to the remainder of the molecule. In some embodiments, (B-I) is

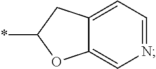 [SMILES: *C1Cc2ccncc2O1]

wherein * represents the attachment point to the remainder of the molecule.

In some embodiments of the compounds of formula (I), or the salts thereof, B is C₆-C₁₄ aryl optionally substituted with 1, 2, 3, 4, 5, 6, 7, 8, or 9 R^{B} substituents. In some embodiments, B is selected from the group consisting of

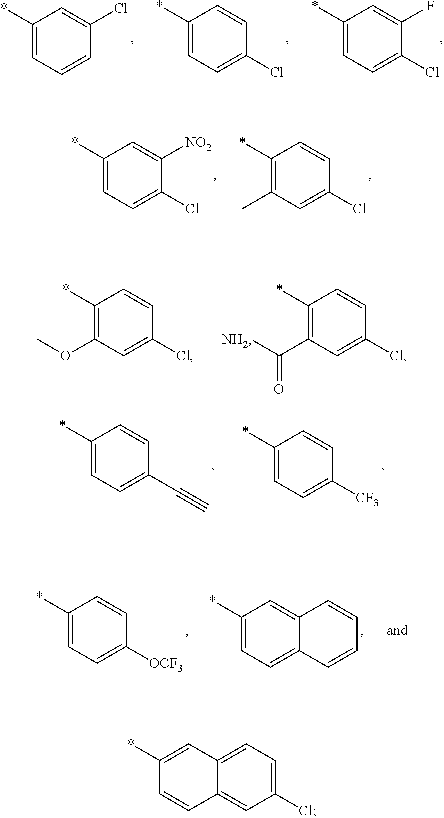

wherein * represents the attachment point to the remainder of the molecule. In some embodiments, B is selected from the group consisting of

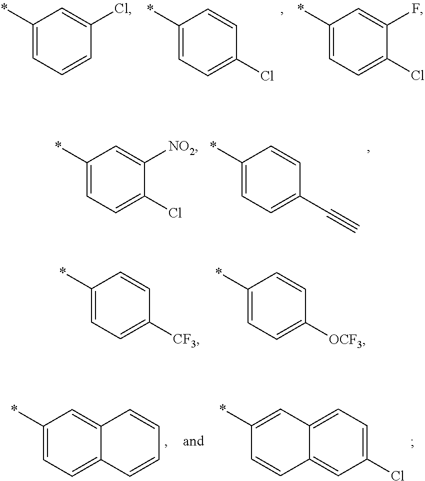

wherein * represents the attachment point to the remainder of the molecule. In some embodiments, B is

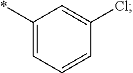 [SMILES: *c1cccc(Cl)c1]

wherein * represents the attachment point to the remainder of the molecule. In some embodiments, B is

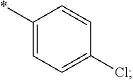 [SMILES: *c1ccc(Cl)cc1]

wherein * represents the attachment point to the remainder of the molecule. In some embodiments, B is

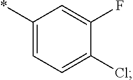 [SMILES: *c1ccc(Cl)c(F)c1]

wherein * represents the attachment point to the remainder of the molecule. In some embodiments, B is

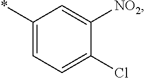 [SMILES: *c1ccc(Cl)c([N+](=O)[O-])c1]

wherein * represents the attachment point to the remainder of the molecule. In some embodiments, B is

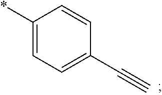 [SMILES: *c1ccc(C#C)cc1]

wherein * represents the attachment point to the remainder of the molecule. In some embodiments, B is

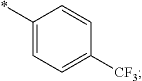 [SMILES: *c1ccc(C(F)(F)F)cc1]

wherein * represents the attachment point to the remainder of the molecule. In some embodiments, B is

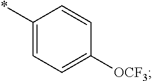 [SMILES: *c1ccc(OC(F)(F)F)cc1]

wherein * represents the attachment point to the remainder of the molecule. In some embodiments, B is

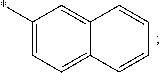 [SMILES: *c1ccc2ccccc2c1]

wherein * represents the attachment point to the remainder of the molecule. In some embodiments, B is

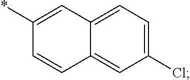 [SMILES: *c1ccc2cc(Cl)ccc2c1]

wherein * represents the attachment point to the remainder of the molecule.

In some embodiments of the compounds of formula (I), or the salts thereof, B is 5-14 membered heteroaryl optionally substituted with 1, 2, 3, 4, 5, 6, 7, 8, or 9 R^{B} substituents. In some embodiments, B is selected from the group consisting of

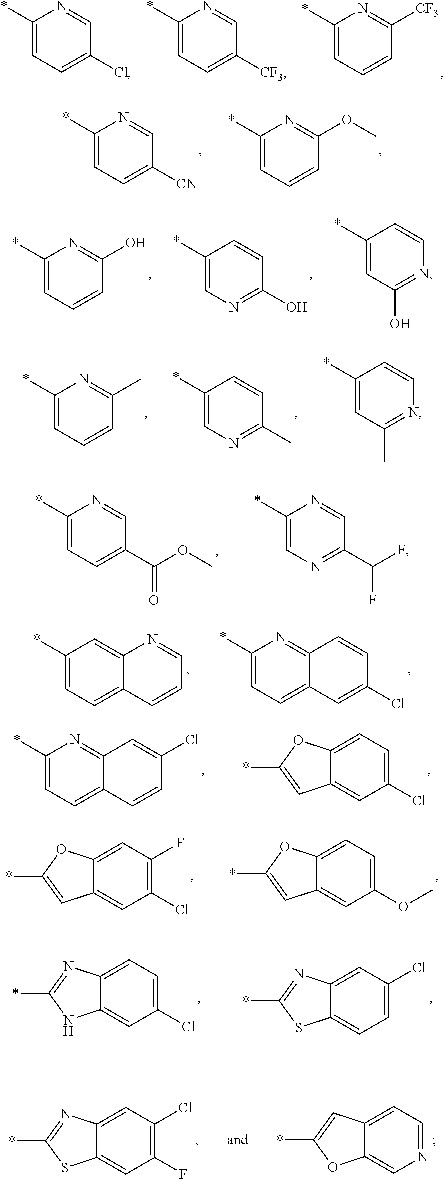

wherein * represents the attachment point to the remainder of the molecule. In some embodiments, B is selected from the group consisting of

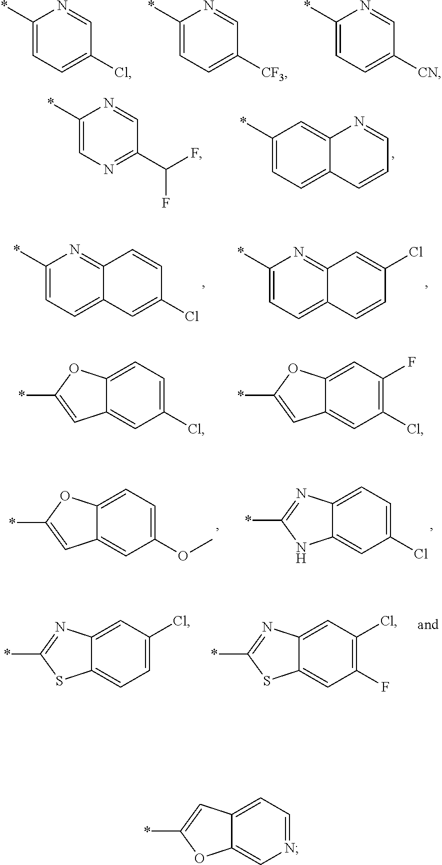

wherein * represents the attachment point to the remainder of the molecule. In some embodiments, B is

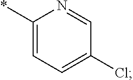 [SMILES: *c1ccc(Cl)cn1]

wherein * represents the attachment point to the remainder of the molecule. In some embodiments, B is

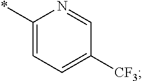 [SMILES: *c1ccc(C(F)(F)F)cn1]

wherein * represents the attachment point to the remainder of the molecule. In some embodiments, B is

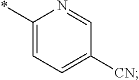 [SMILES: *c1ccc(C#N)cn1]

wherein * represents the attachment point to the remainder of the molecule. In some embodiments, B is

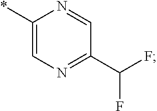 [SMILES: *c1cnc(C(F)F)cn1]

wherein * represents the attachment point to the remainder of the molecule. In some embodiments, B is

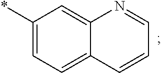 [SMILES: *c1ccc2cccnc2c1]

wherein * represents the attachment point to the remainder of the molecule. In some embodiments, B is

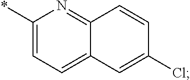 [SMILES: *c1ccc2cc(Cl)ccc2n1]

wherein * represents the attachment point to the remainder of the molecule. In some embodiments, B is

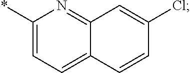 [SMILES: *c1ccc2ccc(Cl)cc2n1]

wherein * represents the attachment point to the remainder of the molecule. In some embodiments, B is

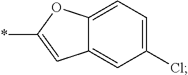 [SMILES: *c1cc2cc(Cl)ccc2o1]

wherein * represents the attachment point to the remainder of the molecule. In some embodiments, B is

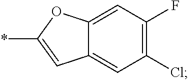 [SMILES: *c1cc2cc(Cl)c(F)cc2o1]

wherein * represents the attachment point to the remainder of the molecule. In some embodiments, B is

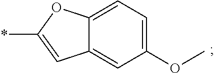 [SMILES: *c1cc2cc(OC)ccc2o1]

wherein * represents the attachment point to the remainder of the molecule. In some embodiments, B is

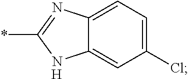

wherein * represents the attachment point to the remainder of the molecule. In some embodiments, B is

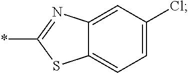 [SMILES: *c1nc2cc(Cl)ccc2s1]

wherein * represents the attachment point to the remainder of the molecule. In some embodiments, B is

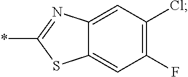 [SMILES: *c1nc2cc(Cl)c(F)cc2s1]

wherein * represents the attachment point to the remainder of the molecule. In some embodiments, B is

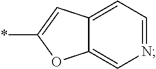 [SMILES: *c1cc2ccncc2o1]

wherein * represents the attachment point to the remainder of the molecule.

In a second aspect, provided is a compound of formula (A-1)

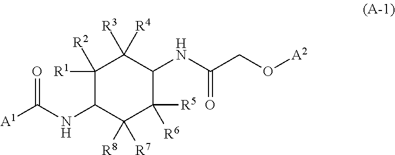

or a pharmaceutically acceptable salt thereof,
wherein:
R¹, R², R³, R⁴, R⁵, R⁶, R⁷, and R⁸, independently from each other, are selected from the group consisting of hydrogen, C₁-C₆ alkyl, C₁-C₆ haloalkyl, —C(O)OH, —C(O)O(C₁-C₆ alkyl), —C(O)O(C₁-C₆ haloalkyl), and halogen;
or, one of R¹, R², R³, R⁴, R⁵, R⁶, R⁷, and R⁸, and another one of R¹, R², R³, R⁴, R⁵, R⁶, R⁷, and R⁸, are taken together to form a C₁-C₆ alkylene moiety;
or, two geminal substituents selected from the group consisting of R¹, R², R³, R⁴, R⁵, R⁶, R⁷, and R⁸ are taken together to form an oxo group;
A¹ is a substituent of formula (A-1)

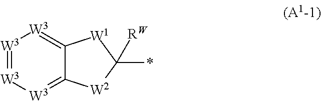

wherein * represents the attachment point to the remainder of the molecule;
   W¹ is selected from the group consisting of —C(R^{W1-1}R^{W1-2})—, —N(R^{W1-2})—, —C(R^{W1-1}R^{W1-1})N(R^{W1-2})—, —N(R^{W1-1})C(R^{W1-1}R^{W1-2})—, —C(R^{W1-1})═N—, —N═C(R^{W1-1})—, —O—, —C(R^{W1-1}R^{W1-1})O—, —OC(R^{W1-1}R^{W1-2})—, —S—, —C(R^{W1-1}R^{W1-1})S—, —SC(R^{W1-1}R^{W1-2})—, —C(R^{W1-1}R^{W1-1})C(R^{W1-1}R^{W1-2})—, and —CR^{W1-1}═CR^{W1-1}—, wherein R^{W1-1} is H or R^{A1}, and R^{W1-2} is H or R^{A1};
   W² is selected from the group consisting of —C(R^{W2-1}R^{W2-2})—, N(R^{W2-2})—, —C(R^{W2-1}R^{W2-1})N(R^{W2-2})—, —N(R^{W2-1})C(R^{W2-1}R^{W2-2})—, —C(R^{W2-1})═N—, —N═C(R^{W2-1})—, —O—, —C(R^{W2-1}R^{W2-1})O—, —OC(R^{W2-1}R^{W2-2})—, —S—, —C(R^{W2-1}R^{W2-1})S, —SC(R^{W2-1}R^{W2-2})—, —C(R^{W2-1}R^{W2-1})C(R^{W2-1}R^{W2-2})—, and —CR^{W2-1}═CR^{W2-1}—, wherein R^{W2-1} is H or R^{A1}, and R^{W2-2} is H or R^{A1};
   W³, independently at each occurrence, is CR^{W3} or N, wherein R^{W3} is H or R^{A1};
   R^{W} is hydrogen or R^{A}, or R^{W} and R^{W1-2} are taken together to form a double bond between the carbon atom bearing R^{W} and the atom bearing R^{W1-2}, or R^{W} and R^{W2-2} are taken together to form a double bond between the carbon atom bearing R^{W} and the atom bearing R^{W2-2};
   R^{A1}, independently at each occurrence, is selected from the group consisting of halogen, NO₂, C₁-C₆ alkyl, C₂-C₆ alkenyl, C₂-C₆ alkynyl, C₁-C₆ haloalkyl, OH, O(C₁-C₆ alkyl), O(C₁-C₆ haloalkyl), SH, S(C₁-C₆ alkyl), S(C₁-C₆ haloalkyl), NH₂, NH(C₁-C₆ alkyl), NH(C₁-C₆ haloalkyl), N(C₁-C₆ alkyl)₂, N(C₁-C₆ haloalkyl)₂, NR^{a}R^{b}, CN, C(O)OH, C(O)O(C₁-C₆ alkyl), C(O)O(C₁-C₆ haloalkyl), C(O)NH₂, C(O)NH(C₁-C₆ alkyl), C(O)NH(C₁-C₆ haloalkyl), C(O)N(C₁-C₆ alkyl)₂, C(O)N(C₁-C₆ haloalkyl)₂, C(O)NR^{a}R^{b}, S(O)₂OH, S(O)₂O(C₁-C₆ alkyl), S(O)₂O(C₁-C₆ haloalkyl), S(O)₂NH₂, S(O)₂NH(C₁-C₆ alkyl), S(O)₂NH(C₁-C₆ haloalkyl), S(O)₂N(C₁-C₆ alkyl)₂, S(O)₂N(C₁-C₆ haloalkyl)₂, S(O)₂NR^{a}R^{b}, OC(O)H, OC(O)(C₁-C₆ alkyl), OC(O)(C₁-C₆ haloalkyl), N(H)C(O)H, N(H)C(O)(C₁-C₆ alkyl), N(H)C(O)(C₁-C₆ haloalkyl), N(C₁-C₆ alkyl)C(O)H, N(C₁-C₆ alkyl)C(O)(C₁-C₆ alkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ alkyl), OS(O)₂(C₁-C₆ haloalkyl), N(H)S(O)₂(C₁-C₆ alkyl), N(H)S(O)₂(C₁-C₆ haloalkyl), N(C₁-C₆ alkyl)S(O)₂(C₁-C₆ alkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl); wherein R^{a} and R^{b} are taken together with the nitrogen atom to which they are attached to form a 3-10 membered heterocycle;
      and
A² is selected from the group consisting of:
   C₆-C₁₄ aryl optionally substituted with 1, 2, 3, 4, 5, 6, 7, 8, or 9 R² substituents; and
   5-14 membered heteroaryl optionally substituted with 1, 2, 3, 4, 5, 6, 7, 8, or 9 R^{A2} substituents;
   R^{A2}, independently at each occurrence, is selected from the group consisting of halogen, NO₂, C₁-C₆ alkyl, C₂-C₆ alkenyl, C₂-C₆ alkynyl, C₁-C₆ haloalkyl, OH, O(C₁-C₆ alkyl), O(C₁-C₆ haloalkyl), SH, S(C₁-C₆ alkyl), S(C₁-C₆ haloalkyl), NH₂, NH(C₁-C₆ alkyl), NH(C₁-C₆ haloalkyl), N(C₁-C₆ alkyl)₂, N(C₁-C₆ haloalkyl)₂, NR^{a}R^{b}, CN, C(O)OH, C(O)O(C₁-C₆ alkyl), C(O)O(C₁-C₆ haloalkyl), C(O)NH₂, C(O)NH(C₁-C₆ alkyl), C(O)NH(C₁-C₆ haloalkyl), C(O)N(C₁-C₆ alkyl)₂, C(O)N(C₁-C₆ haloalkyl)₂, C(O)NR^{a}R^{b}, S(O)₂OH, S(O)₂O(C₁-C₆ alkyl), S(O)₂O(C₁-C₆ haloalkyl), S(O)₂NH₂, S(O)₂NH(C₁-C₆ alkyl), S(O)₂NH(C₁-C₆ haloalkyl), S(O)₂N(C₁-C₆ alkyl)₂, S(O)₂N(C₁-C₆ haloalkyl)₂, S(O)₂NR^{a}R^{b}, OC(O)H, OC(O)(C₁-C₆ alkyl), OC(O)(C₁-C₆ haloalkyl), N(H)C(O)H, N(H)C(O)(C₁-C₆ alkyl), N(H)C(O)(C₁-C₆ haloalkyl), N(C₁-C₆ alkyl)C(O)H, N(C₁-C₆ alkyl)C(O)(C₁-C₆ alkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ alkyl), OS(O)₂(C₁-C₆ haloalkyl), N(H)S(O)₂(C₁-C₆ alkyl), N(H)S(O)₂(C₁-C₆ haloalkyl), N(C₁-C₆ alkyl)S(O)₂(C₁-C₆ alkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl); wherein R^{a} and R^{b} are taken together with the nitrogen atom to which they are attached to form a 3-10 membered heterocycle.

In some embodiments of the compounds of formula (A-1), or the salts thereof, R¹, R², R³, R⁴, R⁵, R⁶, R⁷, and R⁸ are each hydrogen. In some embodiments, R⁴ and R⁸ are taken together to form a C₁-C₆ alkylene moiety. In some embodiments, R⁴ and R⁸ are taken together to form a moiety selected from methylene, ethylene, and propylene. In some embodiments, R⁴ and R⁸ are taken together to form a methylene moiety. In some embodiments, R⁴ and R⁸ are taken together to form an ethylene moiety. In some embodiments, R¹, R², R³, R⁵, R⁶, and R⁷ are each hydrogen, and R⁴ and R⁸ are taken together to form a C₁-C₆ alkylene moiety. In some embodiments, R¹, R², R³, R⁵, R⁶, and R⁷ are each hydrogen, and R⁴ and R⁸ are taken together to form a moiety selected from methylene, ethylene, and propylene. In some embodiments, R¹, R², R³, R⁵, R⁶, and R⁷ are each hydrogen, and R⁴ and R⁸ are taken together to form a methylene moiety. In some embodiments, R¹, R², R³, R⁵, R⁶, and R⁷ are each hydrogen, and R⁴ and R⁸ are taken together to form an ethylene moiety.

In some embodiments of the compounds of formula (A-1), or the salts thereof, A is selected from the group consisting of

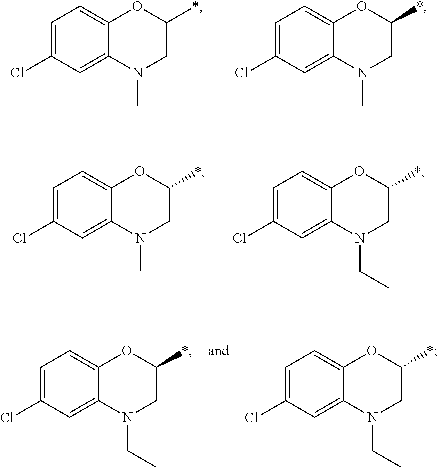

wherein * represents the attachment point to the remainder of the molecule. In some embodiments, (A¹-1) is

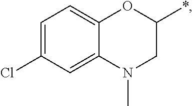

wherein * represents the attachment point to the remainder of the molecule. In some embodiments, (A¹-1) is

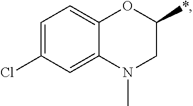

wherein * represents the attachment point to the remainder of the molecule. In some embodiments, (A¹-1) is

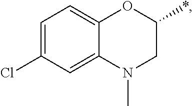

wherein * represents the attachment point to the remainder of the molecule. In some embodiments, (A¹-1) is

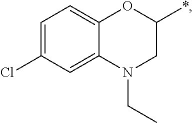

wherein * represents the attachment point to the remainder of the molecule. In some embodiments, (A¹-1) is

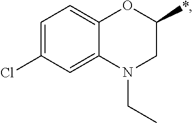

wherein * represents the attachment point to the remainder of the molecule. In some embodiments, (A¹-1) is

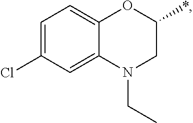

wherein * represents the attachment point to the remainder of the molecule.

In some embodiments of the compounds of formula (A-1), or the salts thereof, A¹ is a substituent of formula (A¹-1)

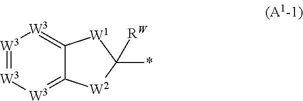

wherein * represents the attachment point to the remainder of the molecule;
W¹ is selected from the group consisting of —C(R^{W1-1}R^{W1-2})—, —N(R^{W1-2})—, —C(R^{W1-1}R^{W1-1})N(R^{W1-2})—, —N(R^{W1-1})C(R^{W1-1}R^{W1-2})—, —C(R^{W1-1})═N—, —N═C(R^{W1-1})—, —O—, —C(R^{W1-1}R^{W1-1})O—, —OC(R^{W1-1}R^{W1-2})—, —S—, —C(R^{W1-1}R^{W1-1})S—, —SC(R^{W1-1}R^{W1-2})—, —C(R^{W1-1}R^{W1-1})C(R^{W1-1}R^{W1-2})—, and —CR^{W1-1}═CR^{W1-1}—,

wherein R^{W1-1} is H or R^{A1}, and R^{W1-2} is H or R^{A1};

W² is selected from the group consisting of —C(R^{W2-1}R^{W2-2})—, —N(R^{W2-2})—, —C(R^{W2-1}R^{W2-1})N(R^{W2-2}), —N(R^{W2-1})C(R^{W2-1}R^{W2-2})—, —C(R^{W2-1})═N—, —N═C(R^{W2-1})—, —O—, —C(R^{W2-1}R^{W2-1})O—, —OC(R^{W2-1}R^{W2-2})—, —S—, —C(R^{W2-1}R^{W2-1})S—, —SC(R^{W2-1}R^{W2-2})—, —C(R^{W2-1}R^{W2-1})C(R^{W2-1}R^{W2-2})—, and —CR^{W2-1}═CR^{W2-1}—,

wherein R^{W2-1} is H or R^{A1}, and R^{W2-2} is H or R^{A1};

W³, independently at each occurrence, is CR^{W3} or N, wherein R^{W3} is H or R^{A1};
R^{W} is hydrogen or R^{A}, or R^{W} and R^{W1-2} are taken together to form a double bond between the carbon atom bearing R^{W} and the atom bearing R^{W1-2}, or R^{W} and R^{W2-2} are taken together to form a double bond between the carbon atom bearing R^{W} and the atom bearing R^{W2-2}.

In some embodiments, (A¹-1) is selected from the group consisting of

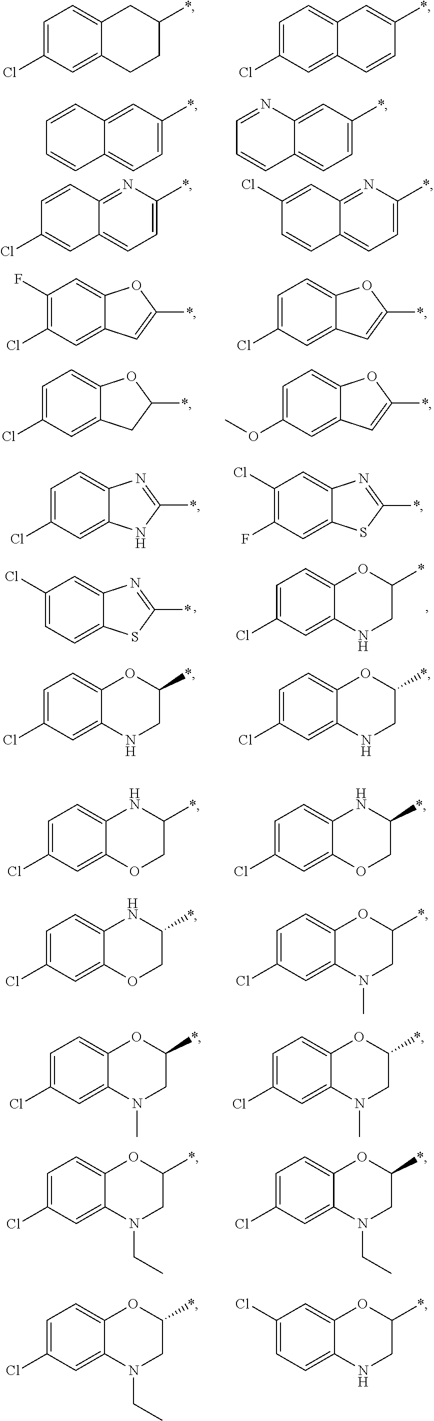

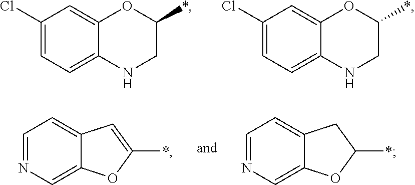

wherein * represents the attachment point to the remainder of the molecule. In some embodiments, (A¹-1) is selected from the group consisting of

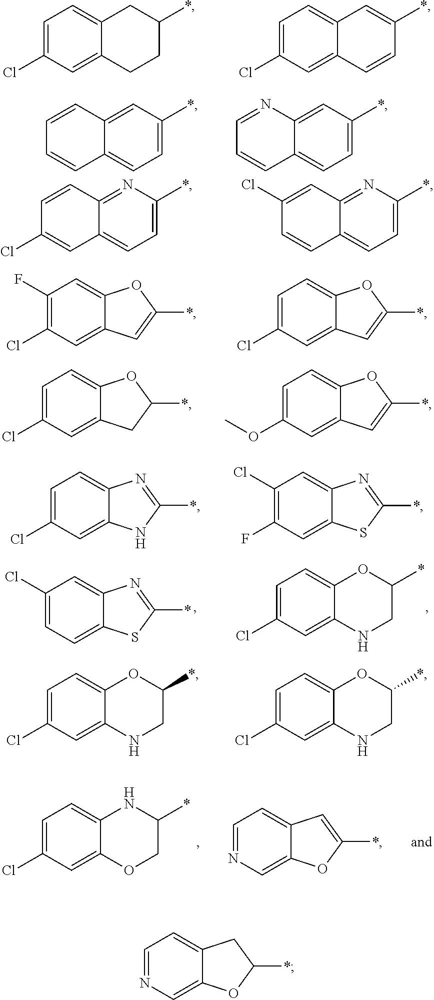

wherein * represents the attachment point to the remainder of the molecule. In some embodiments, (A¹-1) is

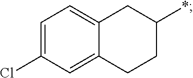

wherein * represents the attachment point to the remainder of the molecule. In some embodiments, (A¹-1) is

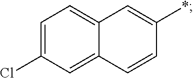

wherein * represents the attachment point to the remainder of the molecule. In some embodiments, (A¹-1) is

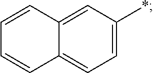

wherein * represents the attachment point to the remainder of the molecule. In some embodiments, (A¹-1) is

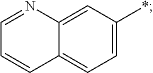

wherein * represents the attachment point to the remainder of the molecule. In some embodiments, (A¹-1) is

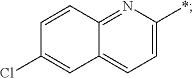

wherein * represents the attachment point to the remainder of the molecule. In some embodiments, (A¹-1) is

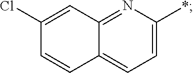

wherein * represents the attachment point to the remainder of the molecule. In some embodiments, (A¹-1) is

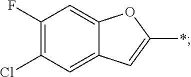

wherein * represents the attachment point to the remainder of the molecule. In some embodiments, (A¹-1) is

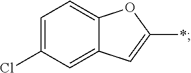

wherein * represents the attachment point to the remainder of the molecule. In some embodiments, (A¹-1) is

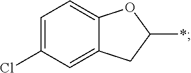

wherein * represents the attachment point to the remainder of the molecule. In some embodiments, (A¹-1) is

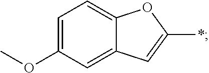

wherein * represents the attachment point to the remainder of the molecule. In some embodiments, (A¹-1) is

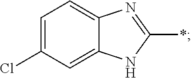

wherein * represents the attachment point to the remainder of the molecule. In some embodiments, (A¹-1) is

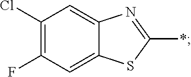

wherein * represents the attachment point to the remainder of the molecule. In some embodiments, (A¹-1) is

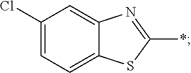

wherein * represents the attachment point to the remainder of the molecule. In some embodiments, (A¹-1) is

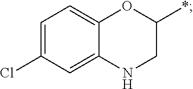

wherein * represents the attachment point to the remainder of the molecule. In some embodiments, (A¹-1) is

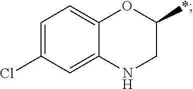

wherein * represents the attachment point to the remainder of the molecule. In some embodiments, (A¹-1) is

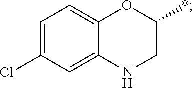

wherein * represents the attachment point to the remainder of the molecule. In some embodiments, (A¹-1) is

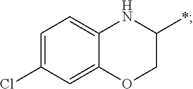

wherein * represents the attachment point to the remainder of the molecule. In some embodiments, (A¹-1) is

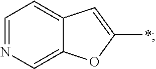

wherein * represents the attachment point to the remainder of the molecule. In some embodiments, (A¹-1) is

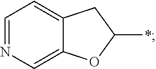

wherein * represents the attachment point to the remainder of the molecule.

In some embodiments of the compounds of formula (A-1), or the salts thereof, A² is C₆-C₁₄ aryl optionally substituted with 1, 2, 3, 4, 5, 6, 7, 8, or 9 R^{A2} substituents. In some embodiments, A² is selected from the group consisting of

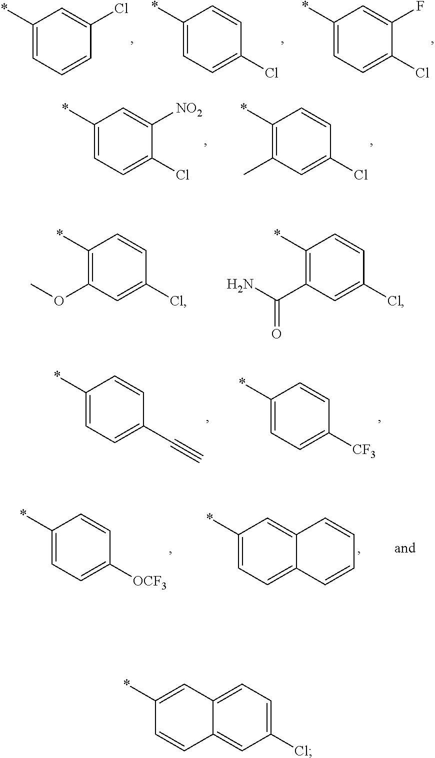

wherein * represents the attachment point to the remainder of the molecule. In some embodiments, A² is selected from the group consisting of

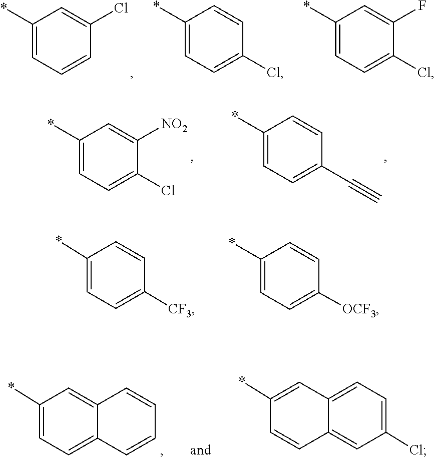

wherein * represents the attachment point to the remainder of the molecule. In some embodiments, A² is

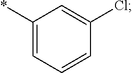 [SMILES: *c1cccc(Cl)c1]

wherein * represents the attachment point to the remainder of the molecule. In some embodiments, A² is

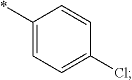 [SMILES: *c1ccc(Cl)cc1]

wherein * represents the attachment point to the remainder of the molecule. In some embodiments, A² is

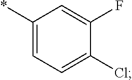 [SMILES: *c1ccc(Cl)c(F)c1]

wherein * represents the attachment point to the remainder of the molecule. In some embodiments, A² is

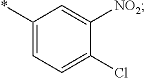 [SMILES: *c1ccc(Cl)c([N+](=O)[O-])c1]

wherein * represents the attachment point to the remainder of the molecule. In some embodiments, A² is

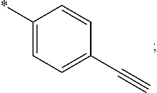 [SMILES: *c1ccc(C#C)cc1]

wherein * represents the attachment point to the remainder of the molecule. In some embodiments, A² is

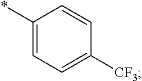 [SMILES: *c1ccc(C(F)(F)F)cc1]

wherein * represents the attachment point to the remainder of the molecule. In some embodiments, A²

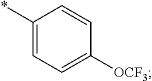 [SMILES: *c1ccc(OC(F)(F)F)cc1]

wherein * represents the attachment point to the remainder of the molecule. In some embodiments, A² is

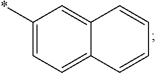 [SMILES: *c1ccc2ccccc2c1]

wherein * represents the attachment point to the remainder of the molecule. In some embodiments, A² is

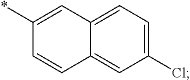 [SMILES: *c1ccc2cc(Cl)ccc2c1]

wherein * represents the attachment point to the remainder of the molecule.

In some embodiments of the compounds of formula (A-1), or the salts thereof, A² is 5-14 membered heteroaryl optionally substituted with 1, 2, 3, 4, 5, 6, 7, 8, or 9 R^{A2} substituents. In some embodiments, A² is selected from the group consisting of

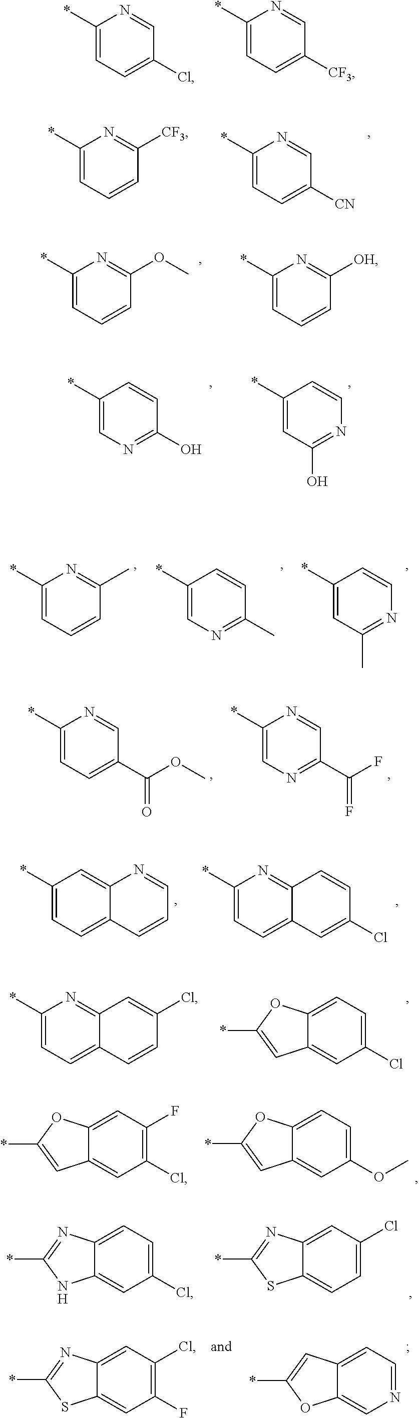

wherein * represents the attachment point to the remainder of the molecule. In some embodiments, A² is selected from the group consisting of

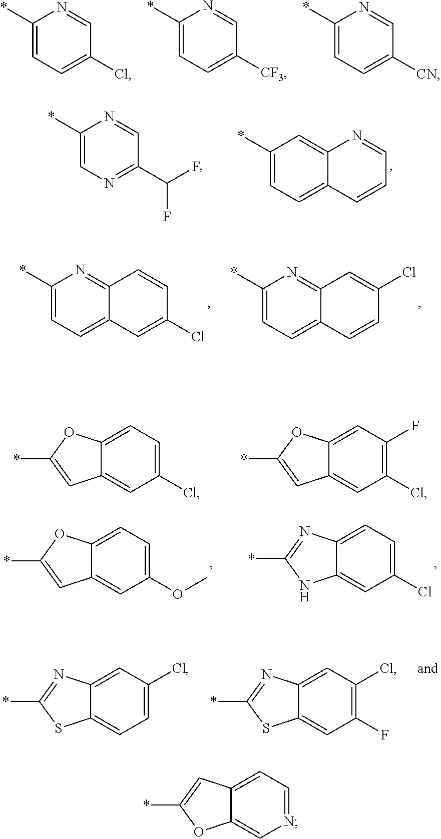

wherein * represents the attachment to the remainder of the molecule. In some embodiments, A² is

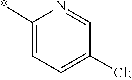 [SMILES: *c1ccc(Cl)cn1]

wherein * represents the attachment point to the remainder of the molecule. In some embodiments, A² is

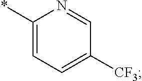 [SMILES: *c1ccc(C(F)(F)F)cn1]

wherein * represents the attachment point to the remainder of the molecule. In some embodiments, A² is

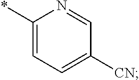 [SMILES: *c1ccc(C#N)cn1]

wherein * represents the attachment point to the remainder of the molecule. In some embodiments, A² is

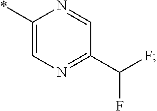 [SMILES: *c1cnc(C(F)F)cn1]

wherein * represents the attachment point to the remainder of the molecule. In some embodiments, A² is

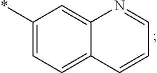 [SMILES: *c1ccc2cccnc2c1]

wherein * represents the attachment point to the remainder of the molecule. In some embodiments, A² is

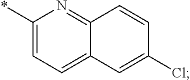 [SMILES: *c1ccc2cc(Cl)ccc2n1]

wherein * represents the attachment point to the remainder of the molecule. In some embodiments, A² is

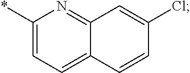 [SMILES: *c1ccc2ccc(Cl)cc2n1]

wherein * represents the attachment point to the remainder of the molecule. In some embodiments, A² is

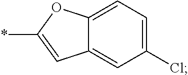 [SMILES: *c1cc2cc(Cl)ccc2o1]

wherein * represents the attachment point to the remainder of the molecule. In some embodiments, A² is

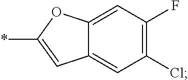 [SMILES: *c1cc2cc(Cl)c(F)cc2o1]

wherein * represents the attachment point to the remainder of the molecule. In some embodiments, A² is

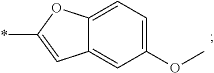 [SMILES: *c1cc2cc(OC)ccc2o1]

wherein * represents the attachment point to the remainder of the molecule. In some embodiments, A² is

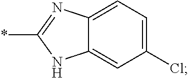 [SMILES: *c1nc2ccc(Cl)cc2[nH]1]

wherein * represents the attachment point to the remainder of the molecule. In some embodiments, A² is

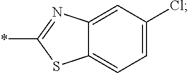 [SMILES: *c1nc2cc(Cl)ccc2s1]

wherein * represents the attachment point to the remainder of the molecule. In some embodiments, A² is

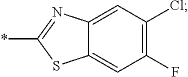 [SMILES: *c1nc2cc(Cl)c(F)cc2s1]

wherein * represents the attachment point to the remainder of the molecule. In some embodiments, A² is

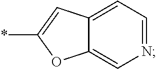 [SMILES: *c1cc2ccncc2o1]

wherein * represents the attachment point to the remainder of the molecule.

In a third aspect, provided is a compound of formula (B-1)

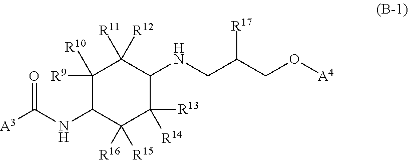

or a pharmaceutically acceptable salt thereof,
wherein:
R⁹, R¹⁰, R¹¹, R¹², R¹³, R¹⁴, R¹⁵, and R¹⁶, independently from each other, are selected from the group consisting of hydrogen, C₁-C₆ alkyl, C₁-C₆ haloalkyl, —C(O)OH, —C(O)O(C₁-C₆ alkyl), —C(O)O(C₁-C₆ haloalkyl), and halogen;
or, one of R⁹, R¹⁰, R¹¹, R¹², R¹³, R¹⁴, R¹⁵, and R¹⁶, and another one of R⁹, R¹⁰, R¹¹, R¹², R¹³, R¹⁴, R¹⁵, and R¹⁶, are taken together to form a C₁-C₆ alkylene moiety;
or, two geminal substituents selected from the group consisting of R⁹, R¹⁰, R¹¹, R¹², R¹³, R¹⁴, R¹⁵, and R¹⁶ are taken together to form an oxo group;
R¹⁷ is H, OH, or NH₂;
A³ is a substituent of formula (A³-1)

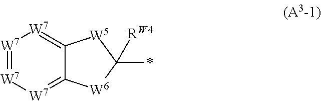

wherein * represents the attachment point to the remainder of the molecule;
   W⁵ is selected from the group consisting of —C(R^{W5-1}R^{W5-2})—, —N(R^{W5-2}), —C(R^{W5-1}R^{W5-2})N(R^{W5-2})—, —N(R^{W5-1})C(R^{W5-1}R^{W5-2})—, —C(R^{W5-1})═N—, —N═C(R^{W5-1}), —O—, —C(R^{W5-1}R^{W5-1})O—, —OC(R^{W5-1}R^{W5-2})—, —S—, —C(R^{W5-1}R^{W5-1})S—, —SC(R^{W5-1}R^{W5-2})—, —C(R^{W5-1}R^{W5-1})C(R^{W5-1}R^{W5-2})—, and —CR^{W5-1}═CR^{W5-1}—, wherein R^{W5-1} is H or R^{A3}, and R^{W5-2} is H or R^{A3};
   W⁶ is selected from the group consisting of —C(R^{W6-1}R^{W6-2})—, —N(R^{W6-2}), —C(R^{W6-1}R^{W6-1})N(R^{W6-2})—, —N(R^{W6-1})C(R^{W6-1}R^{W6-2})—, —C(R^{W6-1})═N—, —N═C(R^{W6-1})—, —O—, —C(R^{W6-1}R^{W6-1})O—, —OC(R^{W6-1}R^{W6-2})—, —S—, —C(R^{W6-1}R^{W6-1})S—, —SC(R^{W6-1}R^{W6-2})—, —C(R^{W6-1}R^{W6-1})C(R^{W6-1}R^{W6-2})—, and —CR^{W6-1}═CR^{W6}—, wherein R^{W6-1} is H or R^{A3}, and R^{W6-2} is H or R^{A3};
   W⁷, independently at each occurrence, is CR^{W7} or N, wherein R^{W7} is H or R^{A};
   R^{A3}, independently at each occurrence, is selected from the group consisting of halogen, NO₂, C₁-C₆ alkyl, C₂-C₆ alkenyl, C₂-C₆ alkynyl, C₁-C₆ haloalkyl, OH, O(C₁-C₆ alkyl), O(C₁-C₆ haloalkyl), SH, S(C₁-C₆ alkyl), S(C₁-C₆ haloalkyl), NH₂, NH(C₁-C₆ alkyl), NH(C₁-C₆ haloalkyl), N(C₁-C₆ alkyl)₂, N(C₁-C₆ haloalkyl)₂, NR^{a}R^{b}, CN, C(O)OH, C(O)O(C₁-C₆ alkyl), C(O)O(C₁-C₆ haloalkyl), C(O)NH₂, C(O)NH(C₁-C₆ alkyl), C(O)NH(C₁-C₆ haloalkyl), C(O)N(C₁-C₆ alkyl)₂, C(O)N(C₁-C₆ haloalkyl)₂, C(O)NR^{a}R^{b}, S(O)₂OH, S(O)₂O(C₁-C₆ alkyl), S(O)₂O(C₁-C₆ haloalkyl), S(O)₂NH₂, S(O)₂NH(C₁-C₆ alkyl), S(O)₂NH(C₁-C₆ haloalkyl), S(O)₂N(C₁-C₆ alkyl)₂, S(O)₂N(C₁-C₆ haloalkyl)₂, S(O)₂NR^{a}R^{b}, OC(O)H, OC(O)(C₁-C₆ alkyl), OC(O)(C₁-C₆ haloalkyl), N(H)C(O)H, N(H)C(O)(C₁-C₆ alkyl), N(H)C(O)(C₁-C₆ haloalkyl), N(C₁-C₆ alkyl)C(O)H, N(C₁-C₆ alkyl)C(O)(C₁-C₆ alkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ alkyl), OS(O)₂(C₁-C₆ haloalkyl), N(H)S(O)₂(C₁-C₆ alkyl), N(H)S(O)₂(C₁-C₆ haloalkyl), N(C₁-C₆ alkyl)S(O)₂(C₁-C₆ alkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl); wherein R^{a} and R^{b} are taken together with the nitrogen atom to which they are attached to form a 3-10 membered heterocycle;
   R^{W4} is hydrogen or R^{A3}, or R^{W4} and R^{W5-2} are taken together to form a double bond between the carbon atom bearing R^{W4} and the atom bearing R^{W5-2}, or R^{W4} and R^{W6-2} are taken together to form a double bond between the carbon atom bearing R^{W} and the atom bearing R^{W6-2};
      and
A⁴ is selected from the group consisting of:
   C₆-C₁₄ aryl optionally substituted with 1, 2, 3, 4, 5, 6, 7, 8, or 9 R^{A4} substituents; and
   5-14 membered heteroaryl optionally substituted with 1, 2, 3, 4, 5, 6, 7, 8, or 9 R^{A4} substituents;
   R^{A4}, independently at each occurrence, is selected from the group consisting of halogen, NO₂, C₁-C₆ alkyl, C₂-C₆ alkenyl, C₂-C₆ alkynyl, C₁-C₆ haloalkyl, OH, O(C₁-C₆ alkyl), O(C₁-C₆ haloalkyl), SH, S(C₁-C₆ alkyl), S(C₁-C₆ haloalkyl), NH₂, NH(C₁-C₆ alkyl), NH(C₁-C₆ haloalkyl), N(C₁-C₆ alkyl)₂, N(C₁-C₆ haloalkyl)₂, NR^{a}R^{b}, CN, C(O)OH, C(O)O(C₁-C₆ alkyl), C(O)O(C₁-C₆ haloalkyl), C(O)NH₂, C(O)NH(C₁-C₆ alkyl), C(O)NH(C₁-C₆ haloalkyl), C(O)N(C₁-C₆ alkyl)₂, C(O)N(C₁-C₆ haloalkyl)₂, C(O)NR^{a}R^{b}, S(O)₂OH, S(O)₂O(C₁-C₆ alkyl), S(O)₂O(C₁-C₆ haloalkyl), S(O)₂NH₂, S(O)₂NH(C₁-C₆ alkyl), S(O)₂NH(C₁-C₆ haloalkyl), S(O)₂N(C₁-C₆ alkyl)₂, S(O)₂N(C₁-C₆ haloalkyl)₂, S(O)₂NR^{a}R^{b}, OC(O)H, OC(O)(C₁-C₆ alkyl), OC(O)(C₁-C₆ haloalkyl), N(H)C(O)H, N(H)C(O)(C₁-C₆ alkyl), N(H)C(O)(C₁-C₆ haloalkyl), N(C₁-C₆ alkyl)C(O)H, N(C₁-C₆ alkyl)C(O)(C₁-C₆ alkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ alkyl), OS(O)₂(C₁-C₆ haloalkyl), N(H)S(O)₂(C₁-C₆ alkyl), N(H)S(O)₂(C₁-C₆ haloalkyl), N(C₁-C₆ alkyl)S(O)₂(C₁-C₆ alkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl); wherein R^{a} and R^{b} are taken together with the nitrogen atom to which they are attached to form a 3-10 membered heterocycle.

In some embodiments of the compounds of formula (B-1), or the salts thereof, R⁹, R¹⁰, R¹¹, R¹², R¹³, R¹⁴, R¹⁵, and R¹⁶ are each hydrogen. In some embodiments, R¹² and R¹⁶ are taken together to form a C₁-C₆ alkylene moiety. In some embodiments, R¹² and R¹⁶ are taken together to form a moiety selected from methylene, ethylene, and propylene. In some embodiments, R¹² and R¹⁶ are taken together to form a methylene moiety. In some embodiments, R¹² and R¹⁶ are taken together to form an ethylene moiety. In some embodiments, R⁹, R¹⁰, R, R¹³, R¹⁴, and R¹⁵ are each hydrogen, and R¹² and R¹⁶ are taken together to form a C₁-C₆ alkylene moiety. In some embodiments, R⁹, R¹⁰, R¹¹, R¹³, R¹⁴, and R¹⁵ are each hydrogen, and R¹² and R¹⁶ are taken together to form a moiety selected from methylene, ethylene, and propylene. In some embodiments, R⁹, R¹⁰, R¹¹, R¹³, R¹⁴, and R¹⁵ are each hydrogen, and R¹² and R¹⁶ are taken together to form a methylene moiety. In some embodiments, R⁹, R¹⁰, R, R¹³, R¹⁴, and R¹⁵ are each hydrogen, and R¹² and R¹⁶ are taken together to form an ethylene moiety.

In some embodiments of the compounds of formula (B-1), or the salts thereof, R¹⁷ is H, OH, or NH₂. In some embodiments, R¹⁷ is OH or NH₂. In some embodiments, R¹⁷ is H. In some embodiments, R¹⁷ is OH. In some embodiments, R¹⁷ is NH₂.

In some embodiments of the compounds of formula (B-1), or the salts thereof, A³ is a substituent of formula (A³-1)

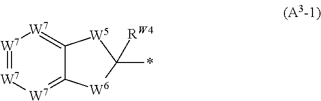

wherein * represents the attachment point to the remainder of the molecule;
W⁵ is selected from the group consisting of —C(R^{W5-1}R^{W5-2})—, —N(R^{W5-2})—, —C(R^{W5-1}R^{W5-2})N(R^{W5-2}), —N(R^{W5-1})C(R^{W5-1}R^{W5-2})—, —C(R^{W5-1})═N—, —N═C(R^{W5-1})—, —O—, —C(R^{W5-1}R^{W5-1})O—, —OC(R^{W5-1}R^{W5-2})—, —S—, —C(R^{W5-1}R^{W5-1})S—, —SC(R^{W5-1}R^{W5-2})—, —C(R^{W5-1}R^{W5-1})C(R^{W5-1}R^{W5-2})—, and —CR^{W5-1}═CR^{W5-1}—,

wherein R^{W5-1} is H or R³, and R^{W5-2} is H or R³;

W⁶ is selected from the group consisting of —C(R^{W6-1}R^{W6-2})—, —N(R^{W6-2})—, —C(R^{W6-1}R^{W6-1})N(R^{W6-2})—, —N(R^{W6-1})C(R^{W6-1}R^{W6-2})—, —C(R^{W6-1})═N—, —N═C(R^{W6-1})—, —O—, —C(R^{W6-1}R^{W6-1})O—, —OC(R^{W6-1}R^{W6-2})—, —S—, —C(R^{W6-1}R^{W6-1})S—, —SC(R^{W6-1}R^{W6-2})—, —C(R^{W6-1}R^{W6-1})C(R^{W6-1}R^{W6-2})—, and —CR^{W6-1}═CR^{W6}—,

wherein R^{W6-1} is H or R³, and R^{W6-2} is H or R^{A3};

W⁷, independently at each occurrence, is CR^{W7} or N, wherein R^{W7} is H or R^{A3}; R^{W4} is hydrogen or R^{A3}, or R^{W4} and R^{W5-2} are taken together to form a double bond between the carbon atom bearing R^{W4} and the atom bearing R^{W5-2}, or R^{W4} and R^{W6-2} are taken together to form a double bond between the carbon atom bearing R^{W} and the atom bearing R^{W6-2}.

In some embodiments, (A³-1) is selected from the group consisting of

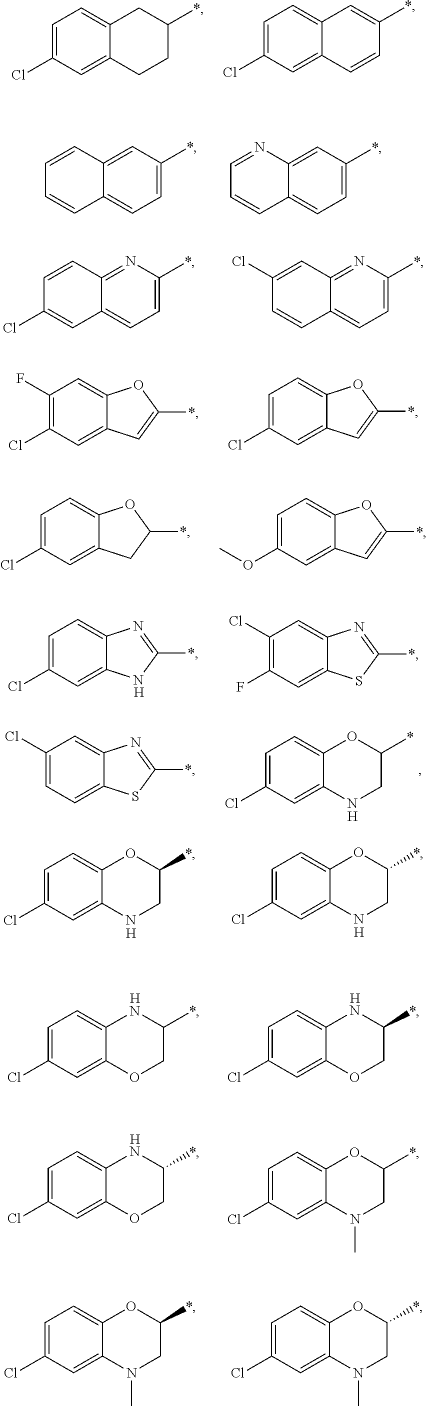

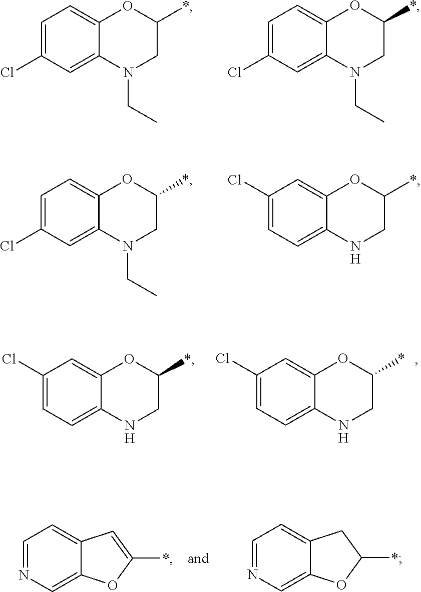

wherein * represents the attachment point to the remainder of the molecule. In some embodiments (A³-1) is selected from the group consisting of

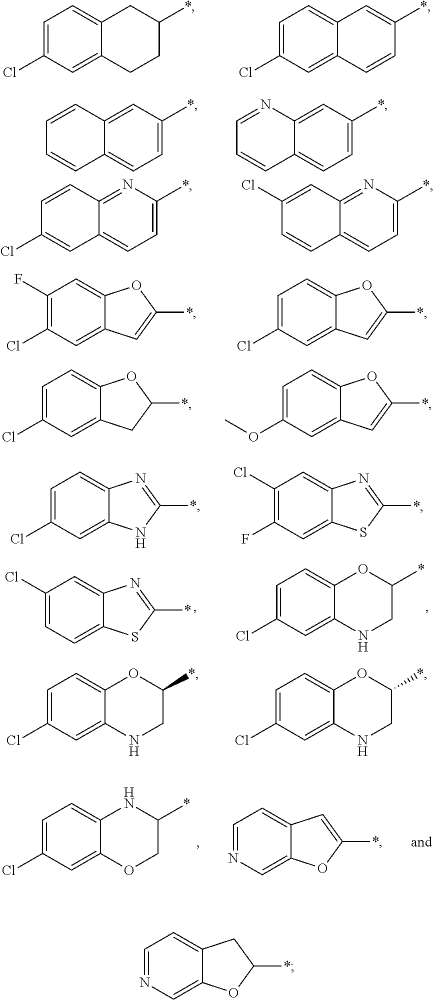

wherein * represents the attachment point to the remainder of the molecule. In some embodiments, (A³-1) is

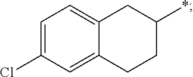

wherein * represents the attachment point to the remainder of the molecule. In some embodiments, (A³-1) is

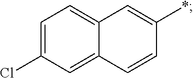

wherein represents the attachment point to the remainder of the molecule. In some embodiments, (A³-1) is

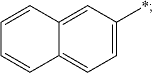

wherein * represents the attachment point to the remainder of the molecule. In some embodiments, (A³-1) is

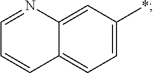

wherein * represents the attachment point to the remainder of the molecule. In some embodiments, (A³-1) is

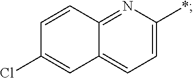

wherein * represents the attachment point to the remainder of the molecule. In some embodiments, (A³-1) is

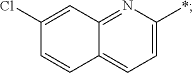

wherein * represents the attachment point to the remainder of the molecule. In some embodiments, (A³-1) is

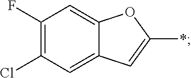

wherein * represents the attachment point to the remainder of the molecule. In some embodiments, (A³-1) is

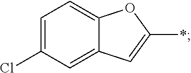

wherein * represents the attachment point to the remainder of the molecule. In some embodiments, (A³-1) is

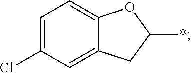

wherein * represents the attachment point to the remainder of the molecule. In some embodiments, (A³-1) is

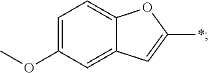

wherein * represents the attachment point to the remainder of the molecule. In some embodiments, (A³-1) is

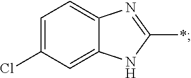

wherein * represents the attachment point to the remainder of the molecule. In some embodiments, (A³-1) is

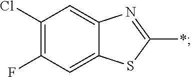

wherein * represents the attachment point to the remainder of the molecule. In some embodiments, (A³-1) is

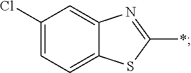

wherein * represents the attachment point to the remainder of the molecule. In some embodiments, (A³-1) is

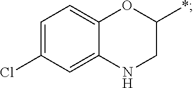

wherein * represents the attachment point to the remainder of the molecule. In some embodiments, (A³-1) is

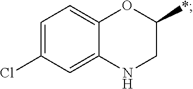

wherein * represents the attachment point to the remainder of the molecule. In some embodiments, (A³-1) is

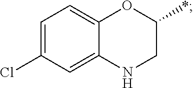

wherein * represents the attachment point to the remainder of the molecule. In some embodiments, (A³-1) is

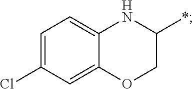

wherein * represents the attachment point to the remainder of the molecule. In some embodiments, (A³-1) is

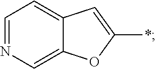

wherein * represents the attachment point to the remainder of the molecule. In some embodiments, (A³-1) is

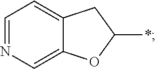

wherein * represents the attachment point to the remainder of the molecule.

In some embodiments of the compounds of formula (B-1), or the salts thereof, A⁴ is C₆-C₁₄ aryl optionally substituted with 1, 2, 3, 4, 5, 6, 7, 8, or 9 R^{A4} substituents. In some embodiments, A⁴ is selected from the group consisting of

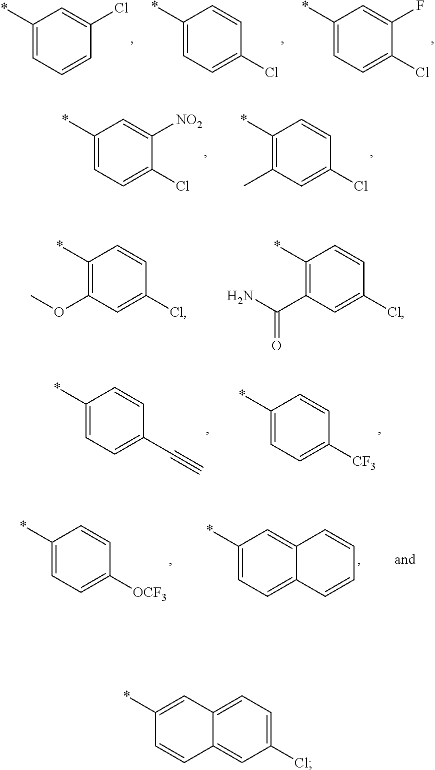

wherein * represents the attachment point to the remainder of the molecule. In some embodiments, A⁴ is selected from the group consisting of

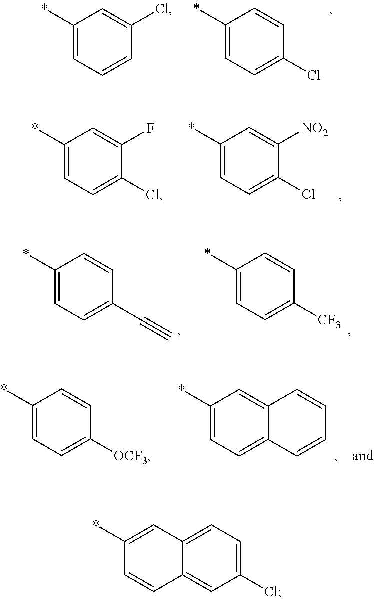

wherein * represents the attachment point to the remainder of the molecule. In some embodiments, A⁴ is

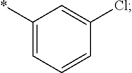 [SMILES: *c1cccc(Cl)c1]

wherein * represents the attachment point to the remainder of the molecule. In some embodiments, A⁴ is

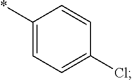 [SMILES: *c1ccc(Cl)cc1]

wherein * represents the attachment point to the remainder of the molecule. In some embodiments, A⁴ is

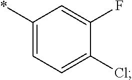 [SMILES: *c1ccc(Cl)c(F)c1]

wherein * represents the attachment point to the remainder of the molecule. In some embodiments, A⁴ is

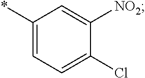 [SMILES: *c1ccc(Cl)c([N+](=O)[O-])c1]

wherein * represents the attachment point to the remainder of the molecule. In some embodiments, A⁴ is

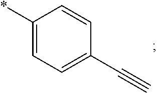 [SMILES: *c1ccc(C#C)cc1]

wherein * represents the attachment point to the remainder of the molecule. In some embodiments, A⁴ is

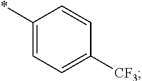 [SMILES: *c1ccc(C(F)(F)F)cc1]

wherein * represents the attachment point to the remainder of the molecule. In some embodiments, A⁴ is

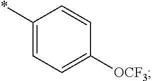 [SMILES: *c1ccc(OC(F)(F)F)cc1]

wherein * represents the attachment point to the remainder of the molecule. In some embodiments, A⁴ is

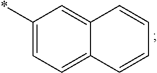 [SMILES: *c1ccc2ccccc2c1]

wherein * represents the attachment point to the remainder of the molecule. In some embodiments, A⁴ is

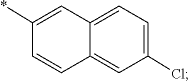 [SMILES: *c1ccc2cc(Cl)ccc2c1]

wherein * represents the attachment point to the remainder of the molecule.

In some embodiments of the compounds of formula (B-1), or the salts thereof, A⁴ is 5-14 membered heteroaryl optionally substituted with 1, 2, 3, 4, 5, 6, 7, 8, or 9 R^{A4} substituents. In some embodiments, A⁴ is selected from the group consisting of

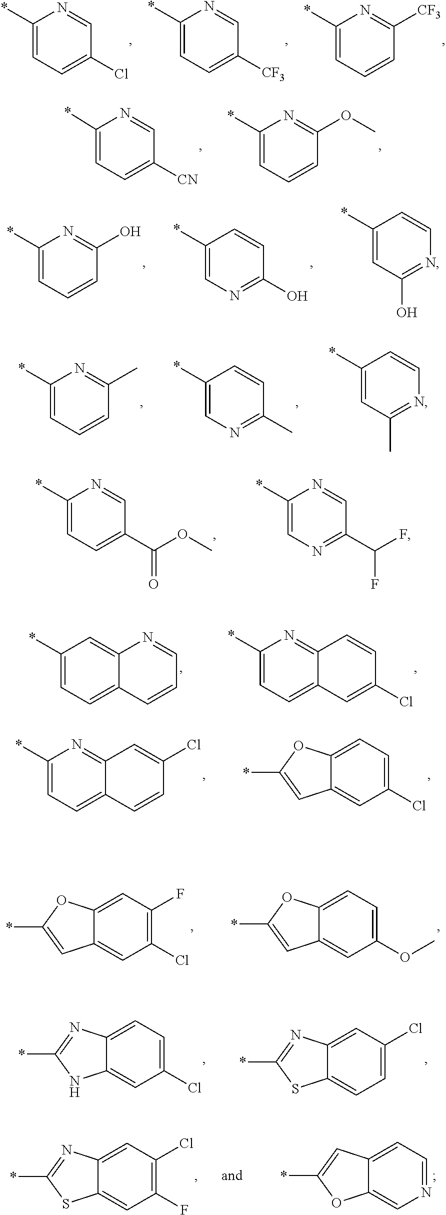

wherein * represents the attachment point to the remainder of the molecule. In some embodiments, A⁴ is selected from the group consisting of

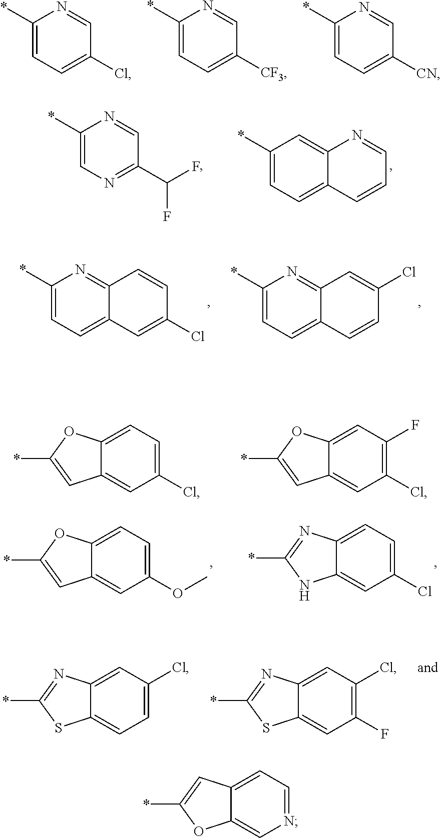

wherein * represents the attachment point to the remainder of the molecule. In some embodiments, A⁴ is

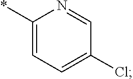 [SMILES: *c1ccc(Cl)cn1]

wherein * represents the attachment point to the remainder of the molecule. In some embodiments, A⁴ is

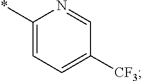 [SMILES: *c1ccc(C(F)(F)F)cn1]

wherein * represents the attachment point to the remainder of the molecule. In some embodiments, A⁴ is

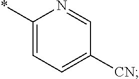 [SMILES: *c1ccc(C#N)cn1]

wherein * represents the attachment point to the remainder of the molecule. In some embodiments, A⁴ is

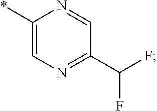 [SMILES: *c1cnc(C(F)F)cn1]

wherein * represents the attachment point to the remainder of the molecule. In some embodiments, A⁴ is

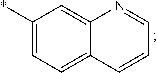 [SMILES: *c1ccc2cccnc2c1]

wherein * represents the attachment point to the remainder of the molecule. In some embodiments, A⁴ is

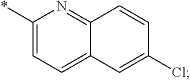 [SMILES: *c1ccc2cc(Cl)ccc2n1]

wherein * represents the attachment point to the remainder of the molecule. In some embodiments, A⁴ is

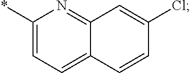 [SMILES: *c1ccc2ccc(Cl)cc2n1]

wherein * represents the attachment point to the remainder of the molecule. In some embodiments, A⁴ is

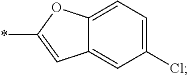 [SMILES: *c1cc2cc(Cl)ccc2o1]

wherein * represents the attachment point to the remainder of the molecule. In some embodiments, A⁴ is

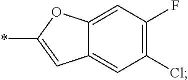 [SMILES: *c1cc2cc(Cl)c(F)cc2o1]

wherein * represents the attachment point to the remainder of the molecule. In some embodiments, A⁴ is

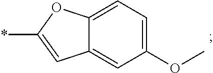 [SMILES: *c1cc2cc(OC)ccc2o1]

wherein * represents the attachment point to the remainder of the molecule. In some embodiments, A⁴ is

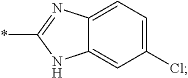 [SMILES: *c1nc2ccc(Cl)cc2[nH]1]

wherein * represents the attachment point to the remainder of the molecule. In some embodiments, A⁴ is

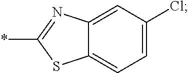 [SMILES: *c1nc2cc(Cl)ccc2s1]

wherein * represents the attachment point to the remainder of the molecule. In some embodiments, A⁴ is

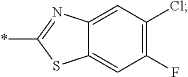 [SMILES: *c1nc2cc(Cl)c(F)cc2s1]

wherein * represents the attachment point to the remainder of the molecule. In some embodiments, A⁴ is

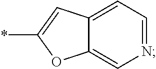 [SMILES: *c1cc2ccncc2o1]

wherein * represents the attachment point to the remainder of the molecule.

In a fourth aspect, provided is a compound of formula (C-1)

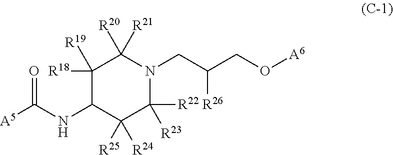

or a pharmaceutically acceptable salt thereof,
wherein:
R¹⁸, R¹⁹, R²⁰, R²¹, R²², R²³, R²⁴, and R², independently from each other, are selected from the group consisting of hydrogen, C₁-C₆ alkyl, C₁-C₆ haloalkyl, —C(O)OH, —C(O)O(C₁-C₆ alkyl), —C(O)O(C₁-C₆ haloalkyl), and halogen;
or, one of R¹⁸, R¹⁹, R²⁰, R²¹, R²², R²³, R²⁴, and R²⁵, and another one of R¹⁸, R¹⁹, R²⁰, R²¹, R²², R²³, R²⁴, and R²⁵, are taken together to form a C₁-C₆ alkylene moiety;
or, two geminal substituents selected from the group consisting of R¹⁸, R¹⁹, R²⁰, R²¹, R²², R²³, R²⁴, and R²⁵ are taken together to form an oxo group;
R²⁶ is H, OH, or NH₂;
A⁵ is a substituent of formula (A⁵-1)

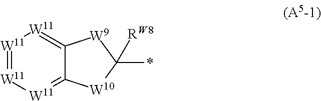

wherein * represents the attachment point to the remainder of the molecule;
   W⁹ is selected from the group consisting of —C(R^{W9-1}R^{W9-2})—, —N(R^{W9-2}), —C(R^{W9-1}R^{W9-2})N(R^{W9-2})—, —N(R^{W9-1})C(R^{W9-1}R^{W9-2})—, —C(R^{W9-1})═N—, —N═C(R^{W9-1})—, —O—, —C(R^{W9-1}R^{W9-1})O—, —OC(R^{W9-1}R^{W9-2})—, —S—, —C(R^{W9-1}R^{W9-1})S—, —SC(R^{W9-1}R^{W9-2})—, —C(R^{W9-1}R^{W9-1})C(R^{W9-1}R^{W9-2})—, and —CR^{W9-1}═CR^{W9-1}, wherein R^{W9-1} is H or R⁵, and R^{W9-2} is H or R⁵;
   W¹⁰ is selected from the group consisting of —C(R^{W10-1}R^{W10-2}), —N(R^{W10-2})—, —C(R^{W10-1}R^{W10-1})N(R^{W10-2})—, —N(R^{W10-1})C(R^{W10-1}R^{W10-2})—, —C(R^{W10-1})═N—, —N═C(R^{W10-1})—, —O—, —C(R^{W10-1}R^{W10-1})O—, —OC(R^{W10-1}R^{W10-2})—, —S—, —C(R^{W10-1}R^{W10-1})S—, —SC(R^{W10-1}R^{W10-2})—, —C(R^{W10-1}R^{W10-1})C(R^{W10-1}R^{W10-2})—, and —CR^{W10-1}═CR^{W10-1}—,
      wherein R^{W10-1} is H or R⁵, and R^{W10-2} is H or R⁵;
   W¹¹, independently at each occurrence, is CR^{W11} or N, wherein R^{W11} is H or R^{A5};
   R^{W8} is hydrogen or R⁵, or R^{W8} and R^{W9-2} are taken together to form a double bond between the carbon atom bearing R^{W8} and the atom bearing R^{W9-2}, or R^{W8} and R^{W10-2} are taken together to form a double bond between the carbon atom bearing R^{W8} and the atom bearing R^{W10-2};
   R⁵, independently at each occurrence, is selected from the group consisting of halogen, NO₂, C₁-C₆ alkyl, C₂-C₆ alkenyl, C₂-C₆ alkynyl, C₁-C₆ haloalkyl, OH, O(C₁-C₆ alkyl), O(C₁-C₆ haloalkyl), SH, S(C₁-C₆ alkyl), S(C₁-C₆ haloalkyl), NH₂, NH(C₁-C₆ alkyl), NH(C₁-C₆ haloalkyl), N(C₁-C₆ alkyl)₂, N(C₁-C₆ haloalkyl)₂, NR^{a}R^{b}, CN, C(O)OH, C(O)O(C₁-C₆ alkyl), C(O)O(C₁-C₆ haloalkyl), C(O)NH₂, C(O)NH(C₁-C₆ alkyl), C(O)NH(C₁-C₆ haloalkyl), C(O)N(C₁-C₆ alkyl)₂, C(O)N(C₁-C₆ haloalkyl)₂, C(O)NR^{a}R^{b}, S(O)₂OH, S(O)₂O(C₁-C₆ alkyl), S(O)₂O(C₁-C₆ haloalkyl), S(O)₂NH₂, S(O)₂NH(C₁-C₆ alkyl), S(O)₂NH(C₁-C₆ haloalkyl), S(O)₂N(C₁-C₆ alkyl)₂, S(O)₂N(C₁-C₆ haloalkyl)₂, S(O)₂NR^{a}R^{b}, OC(O)H, OC(O)(C₁-C₆ alkyl), OC(O)(C₁-C₆ haloalkyl), N(H)C(O)H, N(H)C(O)(C₁-C₆ alkyl), N(H)C(O)(C₁-C₆ haloalkyl), N(C₁-C₆ alkyl)C(O)H, N(C₁-C₆ alkyl)C(O)(C₁-C₆ alkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ alkyl), OS(O)₂(C₁-C₆ haloalkyl), N(H)S(O)₂(C₁-C₆ alkyl), N(H)S(O)₂(C₁-C₆ haloalkyl), N(C₁-C₆ alkyl)S(O)₂(C₁-C₆ alkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl); wherein R^{a} and R^{b} are taken together with the nitrogen atom to which they are attached to form a 3-10 membered heterocycle;
      and
A⁶ is selected from the group consisting of:
   C₆-C₁₄ aryl optionally substituted with 1, 2, 3, 4, 5, 6, 7, 8, or 9 R^{A6} substituents; and
   5-14 membered heteroaryl optionally substituted with 1, 2, 3, 4, 5, 6, 7, 8, or 9 R^{A6} substituents;
   R^{A6}, independently at each occurrence, is selected from the group consisting of halogen, NO₂, C₁-C₆ alkyl, C₂-C₆ alkenyl, C₂-C₆ alkynyl, C₁-C₆ haloalkyl, OH, O(C₁-C₆ alkyl), O(C₁-C₆ haloalkyl), SH, S(C₁-C₆ alkyl), S(C₁-C₆ haloalkyl), NH₂, NH(C₁-C₆ alkyl), NH(C₁-C₆ haloalkyl), N(C₁-C₆ alkyl)₂, N(C₁-C₆ haloalkyl)₂, NR^{a}R^{b}, CN, C(O)OH, C(O)O(C₁-C₆ alkyl), C(O)O(C₁-C₆ haloalkyl), C(O)NH₂, C(O)NH(C₁-C₆ alkyl), C(O)NH(C₁-C₆ haloalkyl), C(O)N(C₁-C₆ alkyl)₂, C(O)N(C₁-C₆ haloalkyl)₂, C(O)NR^{a}R^{b}, S(O)₂OH, S(O)₂O(C₁-C₆ alkyl), S(O)₂O(C₁-C₆ haloalkyl), S(O)₂NH₂, S(O)₂NH(C₁-C₆ alkyl), S(O)₂NH(C₁-C₆ haloalkyl), S(O)₂N(C₁-C₆ alkyl)₂, S(O)₂N(C₁-C₆ haloalkyl)₂, S(O)₂NR^{a}R^{b}, OC(O)H, OC(O)(C₁-C₆ alkyl), OC(O)(C₁-C₆ haloalkyl), N(H)C(O)H, N(H)C(O)(C₁-C₆ alkyl), N(H)C(O)(C₁-C₆ haloalkyl), N(C₁-C₆ alkyl)C(O)H, N(C₁-C₆ alkyl)C(O)(C₁-C₆ alkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ alkyl), OS(O)₂(C₁-C₆ haloalkyl), N(H)S(O)₂(C₁-C₆ alkyl), N(H)S(O)₂(C₁-C₆ haloalkyl), N(C₁-C₆ alkyl)S(O)₂(C₁-C₆ alkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl); wherein R^{a} and R^{b} are taken together with the nitrogen atom to which they are attached to form a 3-10 membered heterocycle.

In some embodiments of the compounds of formula (C-1), or the salts thereof, R¹⁸, R¹⁹, R²⁰, R²¹, R²², R²³, R²⁴, and R²⁵ are each hydrogen. In some embodiments, R²¹ and R²⁵ are taken together to form a C₁-C₆ alkylene moiety. In some embodiments, R²¹ and R²⁵ are taken together to form a moiety selected from methylene, ethylene, and propylene. In some embodiments, R²¹ and R²⁵ are taken together to form a methylene moiety. In some embodiments, R²¹ and R²⁵ are taken together to form an ethylene moiety. In some embodiments, R¹⁸, R¹⁹, R²⁰, R²², R²³, and R²⁴ are each hydrogen, and R²¹ and R²⁵ are taken together to form a C₁-C₆ alkylene moiety. In some embodiments, R¹⁸, R¹⁹, R²⁰, R²², R²³, and R²⁴ are each hydrogen, and R²¹ and R²⁵ are taken together to form a moiety selected from methylene, ethylene, and propylene. In some embodiments, R¹⁸, R¹⁹, R²⁰, R²², R²³, and R²⁴ are each hydrogen, and R²¹ and R²⁵ are taken together to form a methylene moiety. In some embodiments, R¹⁸, R¹⁹, R²⁰, R²², R²³, and R²⁴ are each hydrogen, and R²¹ and R²⁵ are taken together to form an ethylene moiety.

In some embodiments of the compounds of formula (C-1), or the salts thereof, R²⁶ is H, OH, or NH₂. In some embodiments, R²⁶ is OH or NH₂. In some embodiments, R²⁶ is H. In some embodiments, R²⁶ is OH. In some embodiments, R²⁶ is NH₂.

In some embodiments of the compounds of formula (C-1), or the salts thereof, A⁵ is a substituent of formula (A⁵-1)

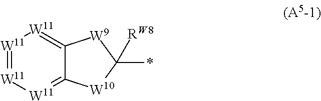

wherein * represents the attachment point to the remainder of the molecule;
W⁹ is selected from the group consisting of —C(R^{W9-1}R^{W9-2})—, —N(R^{W9-2})—, —C(R^{W9-1}R^{W9-2})N(R^{W9-2}), —N(R^{W9-1})C(R^{W9-1}R^{W9-2})—, —C(R^{W9-1})═N—, —N═C(R^{W9-1})—, —O—, —C(R^{W9-1}R^{W9-1})O—, —OC(R^{W9-1}R^{W9-2})—, —S—, —C(R^{W9-1}R^{W9-1})S—, —SC(R^{W9-1}R^{W9-2})—, —C(R^{W9-1}R^{W9-1})C(R^{W9-1}R^{W9-2})—, and —CR^{W9-1}═CR^{W9-1}—,

wherein R^{W9-1} is H or R⁵, and R^{W9-2} is H or R⁵;

W¹⁰ is selected from the group consisting of —C(R^{W10-1}R^{W10-2}), —N(R^{W10-2})—,
—C(R^{W10-1}R^{W10-1})N(R^{W10-2})—, —N(R^{W10-1})C(R^{W10-1}R^{W10-2})—, —C(R^{W10-1})═N—, —N═C(R^{W10-1})—, —O—, —C(R^{W10-1}R^{W10-1})O—, —OC(R^{W10-1}R^{W10-2})—, —S—, —C(R^{W10-1}R^{W10-1})S—, —SC(R^{W10-1}R^{W10-2})—, —C(R^{W10-1}R^{W10-1})C(R^{W10-1}R^{W10-2})—, and —CR^{W10-1}═CR^{W10-1}—,

wherein R^{W10-1} is H or R⁵, and R^{W10-2} is H or R⁵;

W¹¹, independently at each occurrence, is CR^{W11} or N, wherein R^{W11} is H or R^{A5};
R^{W8} is hydrogen or R^{A5}, or R^{W8} and R^{W9-2} are taken together to form a double bond between the carbon atom bearing R^{W8} and the atom bearing R^{W9-2}, or R^{W8} and R^{W10-2} are taken together to form a double bond between the carbon atom bearing R^{W8} and the atom bearing R^{W10-2}.

In some embodiments, (A⁵-1) is selected from the group consisting of

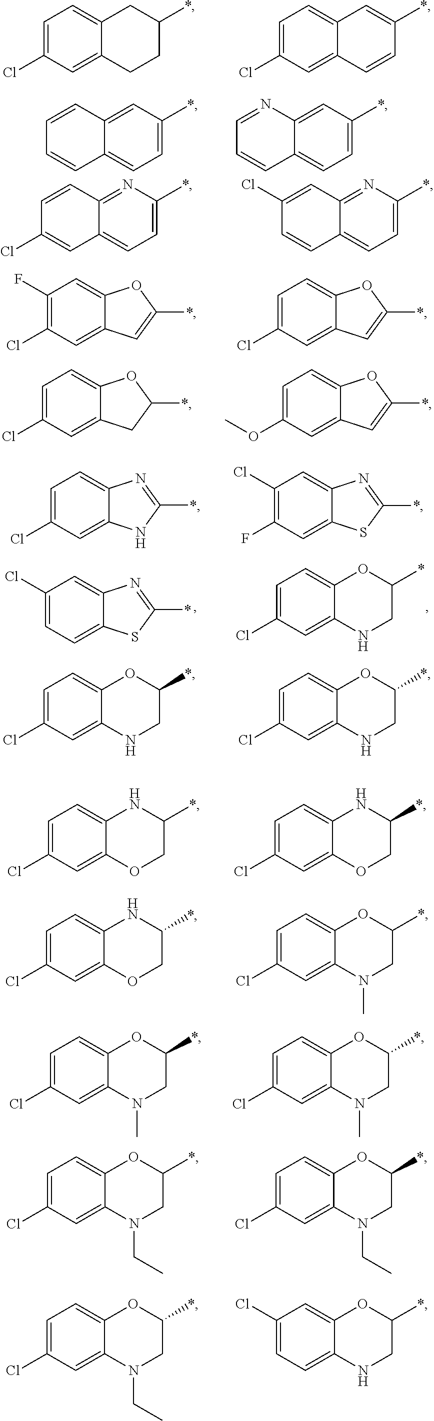

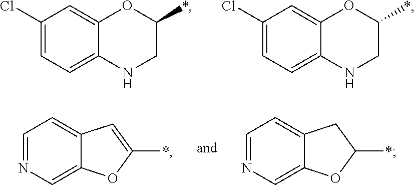

wherein * represents the attachment point to the remainder of the molecule. In some embodiments, (A⁵-1) is selected from the group consisting of

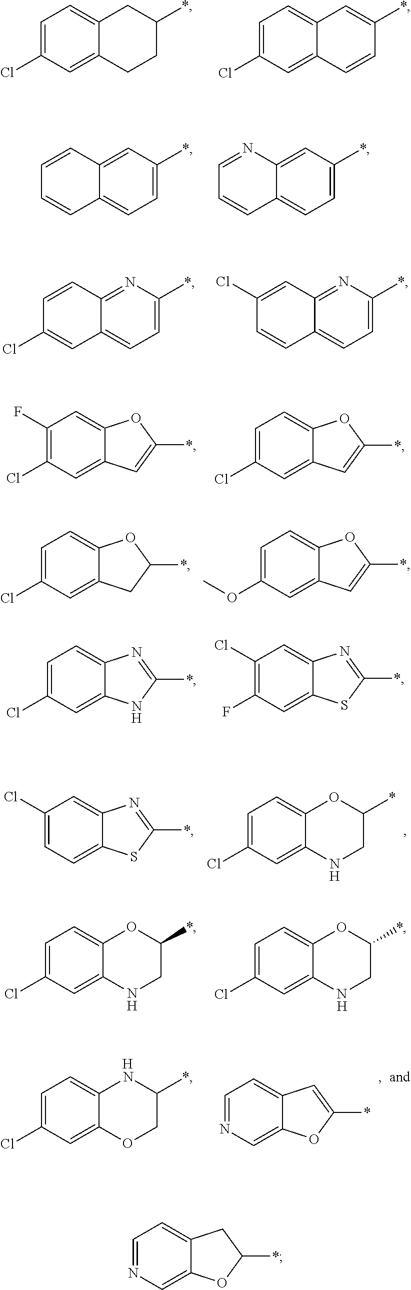

wherein * represents the attachment point to the remainder of the molecule. In some embodiments, (A⁵-1) is

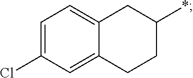

wherein * represents the attachment point to the remainder of the molecule. In some embodiments, (A⁵-1) is

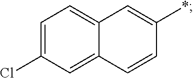

wherein * represents the attachment point to the remainder of the molecule. In some embodiments, (A⁵-1) is

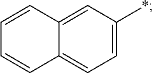

wherein * represents the attachment point to the remainder of the molecule. In some embodiments, (A⁵-1) is

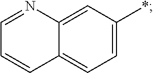

wherein * represents the attachment point to the remainder of the molecule. In some embodiments, (A⁵-1) is

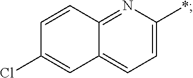

wherein * represents the attachment point to the remainder of the molecule. In some embodiments, (A⁵-1) is

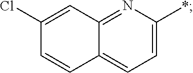

wherein * represents the attachment point to the remainder of the molecule. In some embodiments, (A⁵-1) is

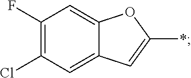

wherein * represents the attachment point to the remainder of the molecule. In some embodiments, (A⁵-1) is

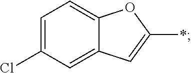

wherein * represents the attachment point to the remainder of the molecule. In some embodiments, (A⁵-1) is

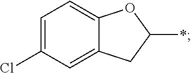

wherein * represents the attachment point to the remainder of the molecule. In some embodiments, (A⁵-1) is

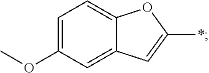

wherein * represents the attachment point to the remainder of the molecule. In some embodiments, (A⁵-1) is

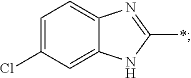

wherein * represents the attachment point to the remainder of the molecule. In some embodiments, (A⁵-1) is

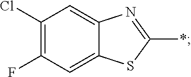

wherein * represents the attachment point to the remainder of the molecule. In some embodiments, (A⁵-1) is

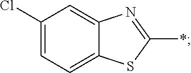

wherein * represents the attachment point to the remainder of the molecule. In some embodiments, (A⁵-1) is

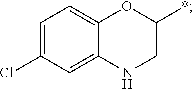

wherein * represents the attachment point to the remainder of the molecule. In some embodiments, (A⁵-1) is

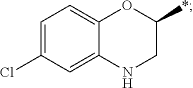

wherein * represents the attachment point to the remainder of the molecule. In some embodiments, (A⁵-1) is

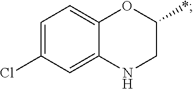

wherein * represents the attachment point to the remainder of the molecule. In some embodiments, (A⁵-1) is

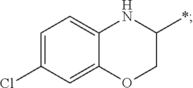

wherein * represents the attachment point to the remainder of the molecule. In some embodiments, (A⁵-1) is

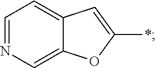

wherein * represents the attachment point to the remainder of the molecule. In some embodiments, (A⁵-1) is

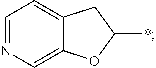

wherein * represents the attachment point to the remainder of the molecule.

In some embodiments of the compounds of formula (C-1), or the salts thereof, A⁶ is C₆-C₁₄ aryl optionally substituted with 1, 2, 3, 4, 5, 6, 7, 8, or 9 R^{A6} substituents. In some embodiments, A⁶ is selected from the group consisting of

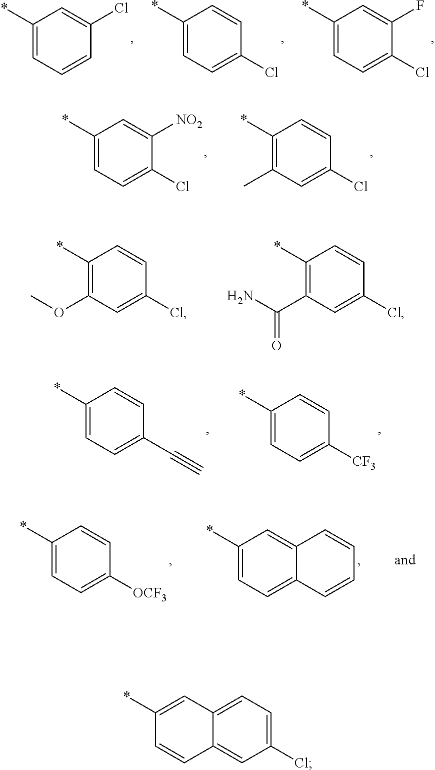

wherein * represents the attachment point to the remainder of the molecule. In some embodiments, A⁶ is selected from the group consisting of

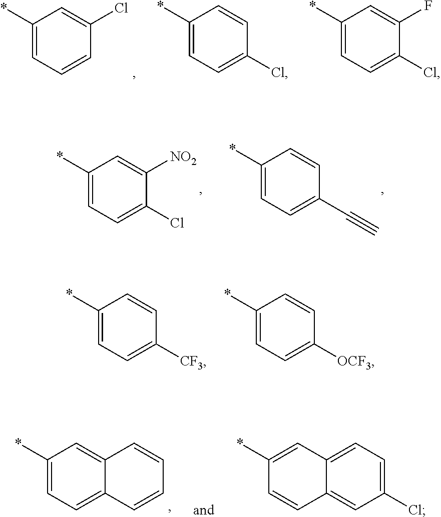

wherein * represents the attachment point to the remainder of the molecule. In some embodiments, A⁶ is

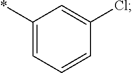 [SMILES: *c1cccc(Cl)c1]

wherein * represents the attachment point to the remainder of the molecule. In some embodiments, A⁶ is

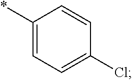 [SMILES: *c1ccc(Cl)cc1]

wherein * represents the attachment point to the remainder of the molecule. In some embodiments, A⁶ is

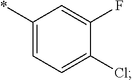 [SMILES: *c1ccc(Cl)c(F)c1]

wherein * represents the attachment point to the remainder of the molecule. In some embodiments, A⁶ is

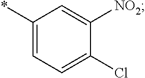 [SMILES: *c1ccc(Cl)c([N+](=O)[O-])c1]

wherein * represents the attachment point to the remainder of the molecule. In some embodiments, A⁶ is

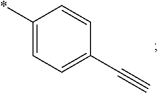 [SMILES: *c1ccc(C#C)cc1]

wherein * represents the attachment point to the remainder of the molecule. In some embodiments, A⁶ is

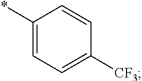 [SMILES: *c1ccc(C(F)(F)F)cc1]

wherein * represents the attachment point to the remainder of the molecule. In some embodiments, A⁶ is

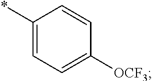 [SMILES: *c1ccc(OC(F)(F)F)cc1]

wherein * represents the attachment point to the remainder of the molecule. In some embodiments, A⁶ is

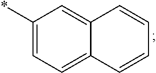 [SMILES: *c1ccc2ccccc2c1]

wherein * represents the attachment point to the remainder of the molecule. In some embodiments, A⁶ is

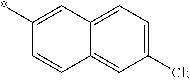 [SMILES: *c1ccc2cc(Cl)ccc2c1]

wherein * represents the attachment point to the remainder of the molecule.

In some embodiments of the compounds of formula (C-1), or the salts thereof, A⁶ is 5-14 membered heteroaryl optionally substituted with 1, 2, 3, 4, 5, 6, 7, 8, or 9 R^{A6} substituents. In some embodiments, A⁶ is selected from the group consisting of

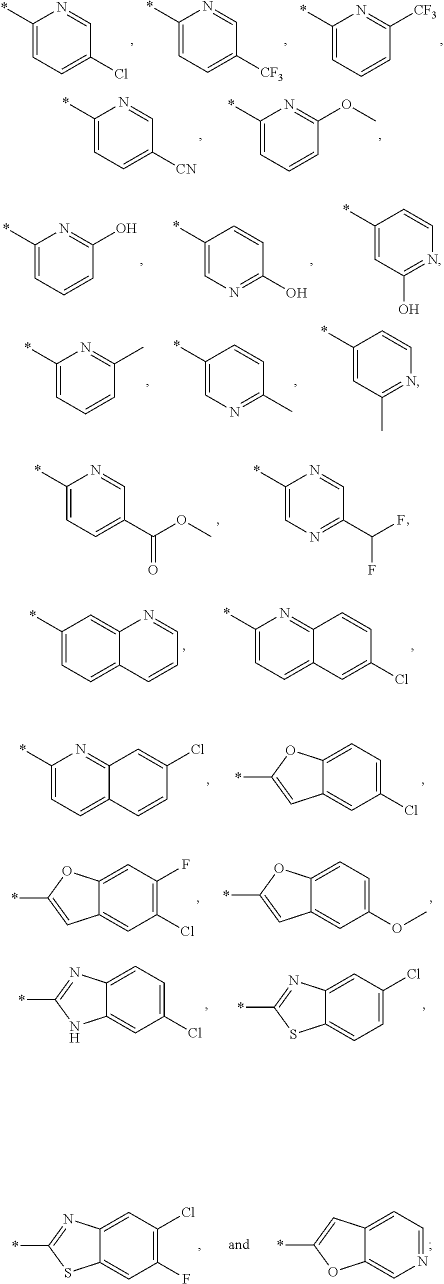

wherein * represents the attachment point to the remainder of the molecule. In some embodiments, A⁶ is selected from the group consisting of

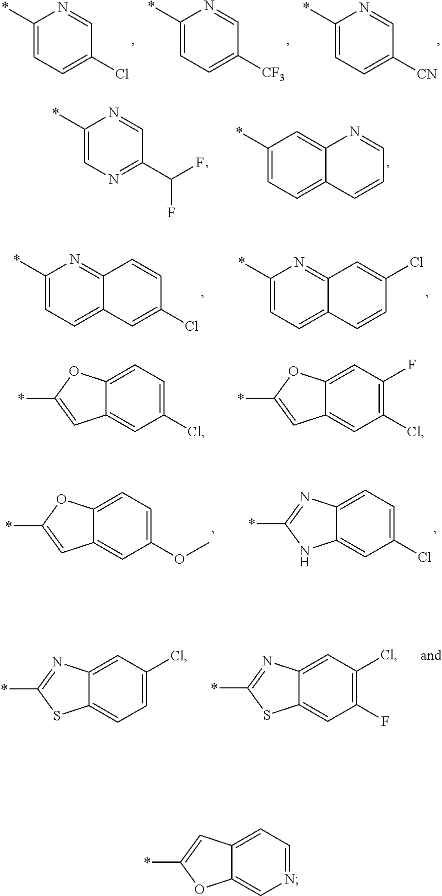

wherein * represents the attachment point to the remainder of the molecule. In some embodiments, A⁶ is

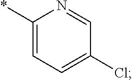 [SMILES: *c1ccc(Cl)cn1]

wherein * represents the attachment point to the remainder of the molecule. In some embodiments, A⁶ is

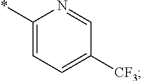 [SMILES: *c1ccc(C(F)(F)F)cn1]

wherein * represents the attachment point to the remainder of the molecule. In some embodiments, A⁶ is

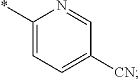 [SMILES: *c1ccc(C#N)cn1]

wherein * represents the attachment point to the remainder of the molecule. In some embodiments, A⁶ is

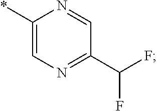 [SMILES: *c1cnc(C(F)F)cn1]

wherein * represents the attachment point to the remainder of the molecule. In some embodiments, A⁶ is

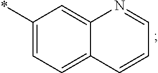 [SMILES: *c1ccc2cccnc2c1]

wherein * represents the attachment point to the remainder of the molecule. In some embodiments, A⁶ is

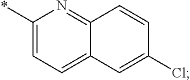 [SMILES: *c1ccc2cc(Cl)ccc2n1]

wherein * represents the attachment point to the remainder of the molecule. In some embodiments, A⁶ is

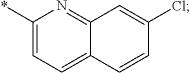 [SMILES: *c1ccc2ccc(Cl)cc2n1]

wherein * represents the attachment point to the remainder of the molecule. In some embodiments, A⁶ is

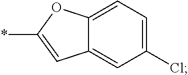 [SMILES: *c1cc2cc(Cl)ccc2o1]

wherein * represents the attachment point to the remainder of the molecule. In some embodiments, A⁶ is

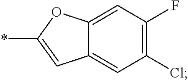 [SMILES: *c1cc2cc(Cl)c(F)cc2o1]

wherein * represents the attachment point to the remainder of the molecule. In some embodiments, A⁶ is

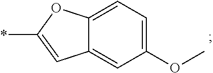 [SMILES: *c1cc2cc(OC)ccc2o1]

wherein * represents the attachment point to the remainder of the molecule. In some embodiments, A⁶ is

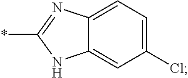 [SMILES: *c1nc2ccc(Cl)cc2[nH]1]

wherein * represents the attachment point to the remainder of the molecule. In some embodiments, A⁶ is

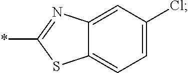 [SMILES: *c1nc2cc(Cl)ccc2s1]

wherein * represents the attachment point to the remainder of the molecule. In some embodiments, A⁶ is

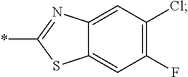 [SMILES: *c1nc2cc(Cl)c(F)cc2s1]

wherein * represents the attachment point to the remainder of the molecule. In some embodiments, A⁶ is

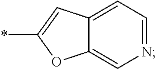 [SMILES: *c1cc2ccncc2o1]

wherein * represents the attachment point to the remainder of the molecule.

In a fifth aspect, provided is a compound of formula (D-1)

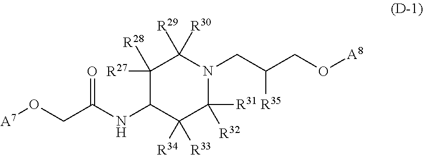

or a pharmaceutically acceptable salt thereof,
wherein:
R²⁷, R²⁸, R²⁹, R³⁰, R³¹, R³², R³³, and R³⁴, independently from each other, are selected from the group consisting of hydrogen, C₁-C₆ alkyl, C₁-C₆ haloalkyl, —C(O)OH, —C(O)O(C₁-C₆ alkyl), —C(O)O(C₁-C₆ haloalkyl), and halogen;
or, one of R²⁷, R²⁸, R²⁹, R³⁰, R³¹, R³², R³³, and R³⁴, and another one of R²⁷, R²⁸, R²⁹, R³⁰, R³¹, R³², R³³, and R³⁴, are taken together to form a C₁-C₆ alkylene moiety;
or, two geminal substituents selected from the group consisting of R²⁷, R²⁸, R²⁹, R³⁰, R³¹, R³², R³³, and R³⁴ are taken together to form an oxo group;
R³⁵ is H, OH, or NH₂;
A⁷ is selected from the group consisting of:
   C₆-C₁₄ aryl optionally substituted with 1, 2, 3, 4, 5, 6, 7, 8, or 9 R^{A7} substituents; and
   5-14 membered heteroaryl optionally substituted with 1, 2, 3, 4, 5, 6, 7, 8, or 9 R^{A7} substituents;
   R^{A7}, independently at each occurrence, is selected from the group consisting of halogen, NO₂, C₁-C₆ alkyl, C₂-C₆ alkenyl, C₂-C₆ alkynyl, C₁-C₆ haloalkyl, OH, O(C₁-C₆ alkyl), O(C₁-C₆ haloalkyl), SH, S(C₁-C₆ alkyl), S(C₁-C₆ haloalkyl), NH₂, NH(C₁-C₆ alkyl), NH(C₁-C₆ haloalkyl), N(C₁-C₆ alkyl)₂, N(C₁-C₆ haloalkyl)₂, NR^{a}R^{b}, CN, C(O)OH, C(O)O(C₁-C₆ alkyl), C(O)O(C₁-C₆ haloalkyl), C(O)NH₂, C(O)NH(C₁-C₆ alkyl), C(O)NH(C₁-C₆ haloalkyl), C(O)N(C₁-C₆ alkyl)₂, C(O)N(C₁-C₆ haloalkyl)₂, C(O)NR^{a}R^{b}, S(O)₂OH, S(O)₂O(C₁-C₆ alkyl), S(O)₂O(C₁-C₆ haloalkyl), S(O)₂NH₂, S(O)₂NH(C₁-C₆ alkyl), S(O)₂NH(C₁-C₆ haloalkyl), S(O)₂N(C₁-C₆ alkyl)₂, S(O)₂N(C₁-C₆ haloalkyl)₂, S(O)₂NR^{a}R^{b}, OC(O)H, OC(O)(C₁-C₆ alkyl), OC(O)(C₁-C₆ haloalkyl), N(H)C(O)H, N(H)C(O)(C₁-C₆ alkyl), N(H)C(O)(C₁-C₆ haloalkyl), N(C₁-C₆ alkyl)C(O)H, N(C₁-C₆ alkyl)C(O)(C₁-C₆ alkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ alkyl), OS(O)₂(C₁-C₆ haloalkyl), N(H)S(O)₂(C₁-C₆ alkyl), N(H)S(O)₂(C₁-C₆ haloalkyl), N(C₁-C₆ alkyl)S(O)₂(C₁-C₆ alkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl); wherein R^{a} and R^{b} are taken together with the nitrogen atom to which they are attached to form a 3-10 membered heterocycle;
      and
A⁸ is selected from the group consisting of:
   C₆-C₁₄ aryl optionally substituted with 1, 2, 3, 4, 5, 6, 7, 8, or 9 R^{A8} substituents; and
   5-14 membered heteroaryl optionally substituted with 1, 2, 3, 4, 5, 6, 7, 8, or 9 R^{A8} substituents;
   R^{A8}, independently at each occurrence, is selected from the group consisting of halogen, NO₂, C₁-C₆ alkyl, C₂-C₆ alkenyl, C₂-C₆ alkynyl, C₁-C₆ haloalkyl, OH, O(C₁-C₆ alkyl), O(C₁-C₆ haloalkyl), SH, S(C₁-C₆ alkyl), S(C₁-C₆ haloalkyl), NH₂, NH(C₁-C₆ alkyl), NH(C₁-C₆ haloalkyl), N(C₁-C₆ alkyl)₂, N(C₁-C₆ haloalkyl)₂, NR^{a}R^{b}, CN, C(O)OH, C(O)O(C₁-C₆ alkyl), C(O)O(C₁-C₆ haloalkyl), C(O)NH₂, C(O)NH(C₁-C₆ alkyl), C(O)NH(C₁-C₆ haloalkyl), C(O)N(C₁-C₆ alkyl)₂, C(O)N(C₁-C₆ haloalkyl)₂, C(O)NR^{a}R^{b}, S(O)₂OH, S(O)₂O(C₁-C₆ alkyl), S(O)₂O(C₁-C₆ haloalkyl), S(O)₂NH₂, S(O)₂NH(C₁-C₆ alkyl), S(O)₂NH(C₁-C₆ haloalkyl), S(O)₂N(C₁-C₆ alkyl)₂, S(O)₂N(C₁-C₆ haloalkyl)₂, S(O)₂NR^{a}R^{b}, OC(O)H, OC(O)(C₁-C₆ alkyl), OC(O)(C₁-C₆ haloalkyl), N(H)C(O)H, N(H)C(O)(C₁-C₆ alkyl), N(H)C(O)(C₁-C₆ haloalkyl), N(C₁-C₆ alkyl)C(O)H, N(C₁-C₆ alkyl)C(O)(C₁-C₆ alkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ alkyl), OS(O)₂(C₁-C₆ haloalkyl), N(H)S(O)₂(C₁-C₆ alkyl), N(H)S(O)₂(C₁-C₆ haloalkyl), N(C₁-C₆ alkyl)S(O)₂(C₁-C₆ alkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl); wherein R^{a} and R^{b} are taken together with the nitrogen atom to which they are attached to form a 3-10 membered heterocycle.

In some embodiments of the compounds of formula (D-1), or the salts thereof, R²⁷, R²⁸, R²⁹, R³⁰, R³¹, R³², R³³, and R³⁴ are each hydrogen. In some embodiments, R³⁰ and R³⁴ are taken together to form a C₁-C₆ alkylene moiety. In some embodiments, R³⁰ and R³⁴ are taken together to form a moiety selected from methylene, ethylene, and propylene. In some embodiments, R³⁰ and R³⁴ are taken together to form a methylene moiety. In some embodiments, R³⁰ and R³⁴ are taken together to form an ethylene moiety. In some embodiments, R²⁷, R²⁸, R²⁹, R³¹, R³², and R³³ are each hydrogen, and R³⁰ and R³⁴ are taken together to form a C₁-C₆ alkylene moiety. In some embodiments, R²⁷, R²⁸, R²⁹, R³¹, R³², and R³³ are each hydrogen, and R³⁰ and R³⁴ are taken together to form a moiety selected from methylene, ethylene, and propylene. In some embodiments, R²⁷, R²⁸, R²⁹, R³¹, R³², and R³³ are each hydrogen, and R³⁰ and R³⁴ are taken together to form a methylene moiety. In some embodiments, R²⁷, R²⁸, R²⁹, R³¹, R³², and R³³ are each hydrogen, and R³⁰ and R³⁴ are taken together to form an ethylene moiety.

In some embodiments of the compounds of formula (D-1), or the salts thereof, R³⁵ is H, OH, or NH₂. In some embodiments, R³⁵ is OH or NH₂. In some embodiments, R³⁵ is H. In some embodiments, R³⁵ is OH. In some embodiments, R³⁵ is NH₂.

In some embodiments of the compounds of formula (D-1), or the salts thereof, A⁷ is C₆-C₁₄ aryl optionally substituted with 1, 2, 3, 4, 5, 6, 7, 8, or 9 R^{A7} substituents. In some embodiments, A⁷ is selected from the group consisting of

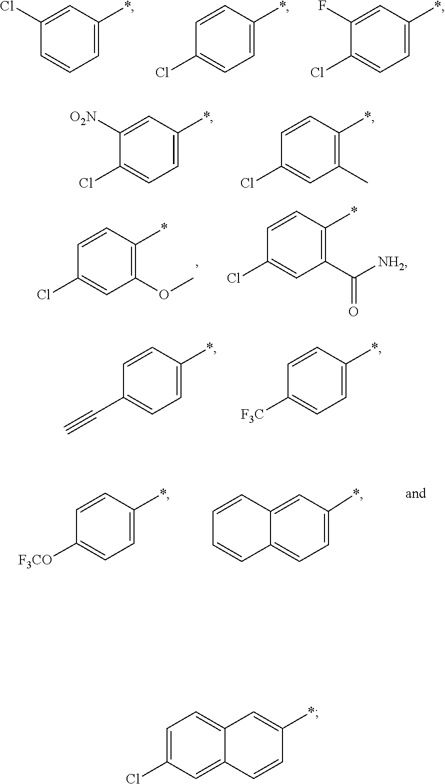

wherein * represents the attachment point to the remainder of the molecule. In some embodiments, A⁷ is selected from the group consisting of

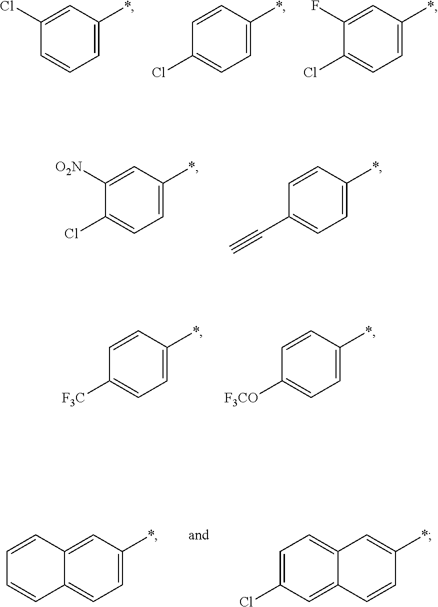

wherein * represents the attachment point to the remainder of the molecule. In some embodiments, A⁷ is

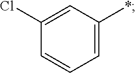

wherein * represents the attachment point to the remainder of the molecule. In some embodiments, A⁷ is

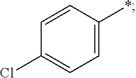

wherein * represents the attachment point to the remainder of the molecule. In some embodiments, A⁷ is

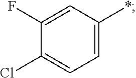

wherein * represents the attachment point to the remainder of the molecule. In some embodiments, A⁷ is

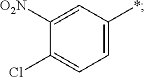

wherein * represents the attachment point to the remainder of the molecule. In some embodiments, A⁷ is

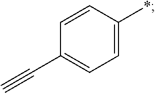

wherein * represents the attachment point to the remainder of the molecule. In some embodiments, A⁷ is

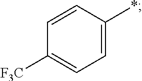

wherein * represents the attachment point to the remainder of the molecule. In some embodiments, A⁷ is

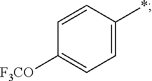

wherein * represents the attachment point to the remainder of the molecule. In some embodiments, A⁷ is

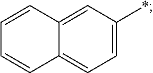

wherein * represents the attachment point to the remainder of the molecule. In some embodiments, A⁷ is

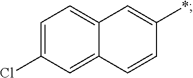

wherein * represents the attachment point to the remainder of the molecule.

In some embodiments of the compounds of formula (D-1), or the salts thereof, A⁷ is 5-14 membered heteroaryl optionally substituted with 1, 2, 3, 4, 5, 6, 7, 8, or 9 R^{A7} substituents. In some embodiments, A⁷ is selected from the group consisting of

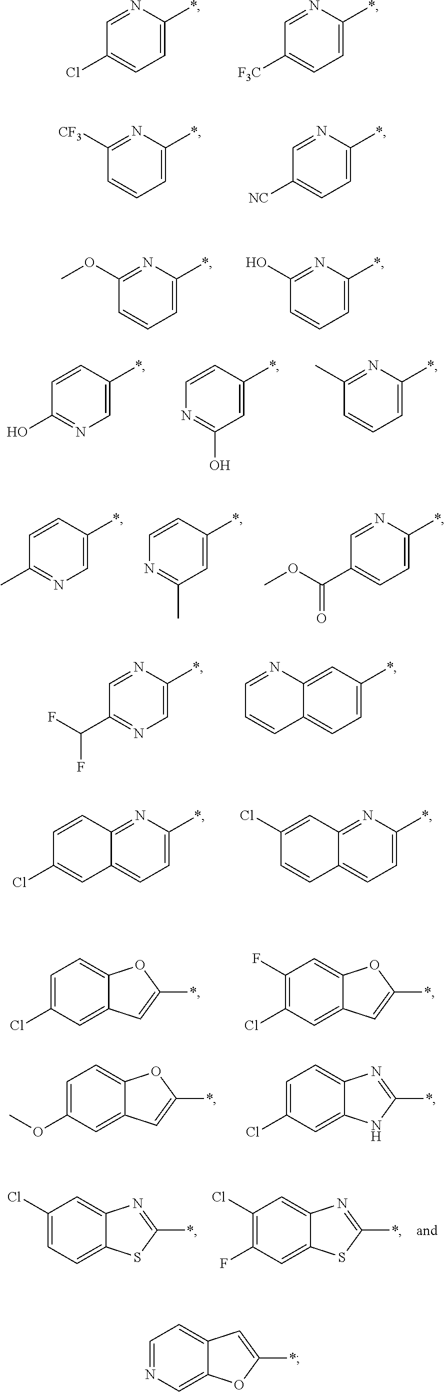

wherein * represents the attachment point to the remainder of the molecule. In some embodiments, A⁷ is selected from the group consisting of

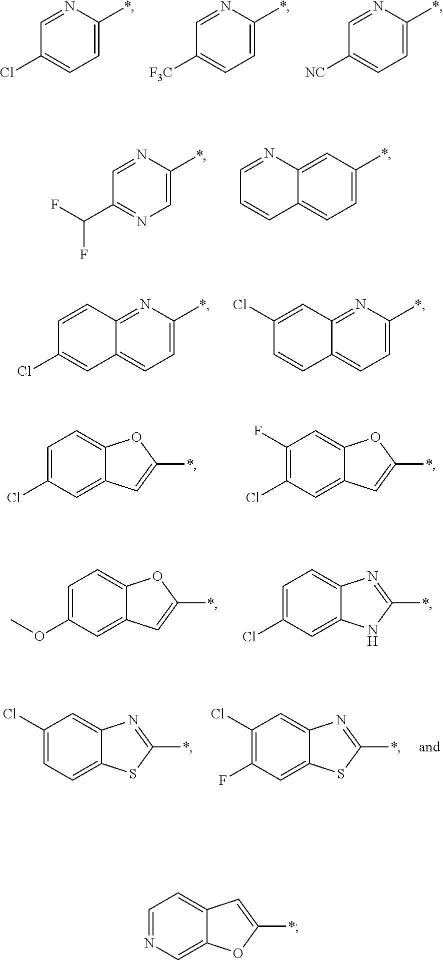

wherein * represents the attachment point to the remainder of the molecule. In some embodiments, A⁷ is

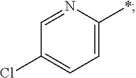

wherein * represents the attachment point to the remainder of the molecule. In some embodiments, A⁷ is

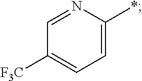

wherein * represents the attachment point to the remainder of the molecule. In some embodiments A is

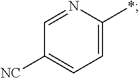

wherein * represents the attachment point to the remainder of the molecule. In some embodiments, A⁷ is

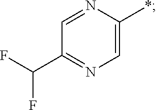

wherein * represents the attachment point to the remainder of the molecule. In some embodiments, A⁷ is

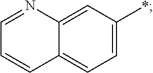

wherein * represents the attachment point to the remainder of the molecule. In some embodiments, A⁷ is

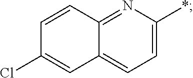

wherein * represents the attachment point to the remainder of the molecule. In some embodiments, A⁷ is

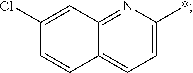

wherein * represents the attachment point to the remainder of the molecule. In some embodiments, A⁷ is

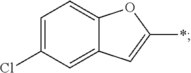

wherein * represents the attachment point to the remainder of the molecule. In some embodiments, A⁷ is

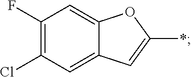

wherein * represents the attachment point to the remainder of the molecule. In some embodiments, A⁷ is

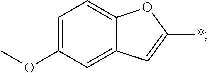

wherein * represents the attachment point to the remainder of the molecule. In some embodiments, A⁷ is

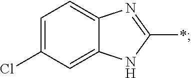

wherein * represents the attachment point to the remainder of the molecule. In some embodiments, A⁷ is

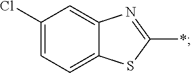

wherein * represents the attachment point to the remainder of the molecule. In some embodiments, A⁷ is

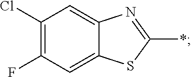

wherein * represents the attachment point to the remainder of the molecule. In some embodiments, A⁷ is

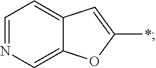

wherein * represents the attachment point to the remainder of the molecule.

In some embodiments of the compounds of formula (D-1), or the salts thereof, A⁸ is C₆-C₁₄ aryl optionally substituted with 1, 2, 3, 4, 5, 6, 7, 8, or 9 R^{A8} substituents. In some embodiments, A⁸ is selected from the group consisting of

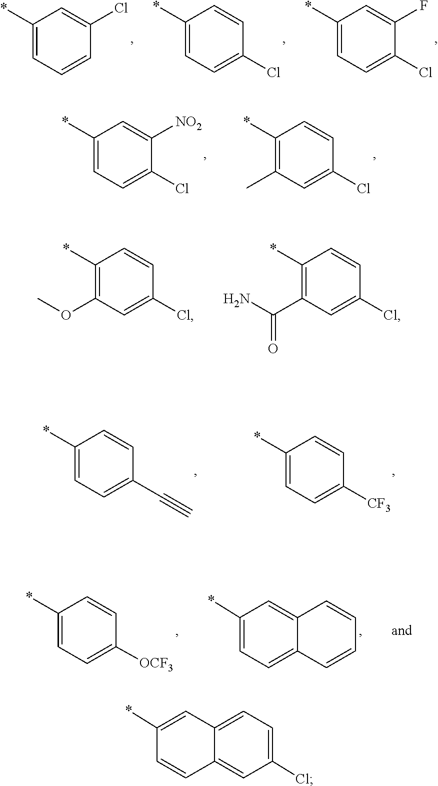

wherein * represents the attachment point to the remainder of the molecule. In some embodiments, A⁸ is selected from the group consisting of

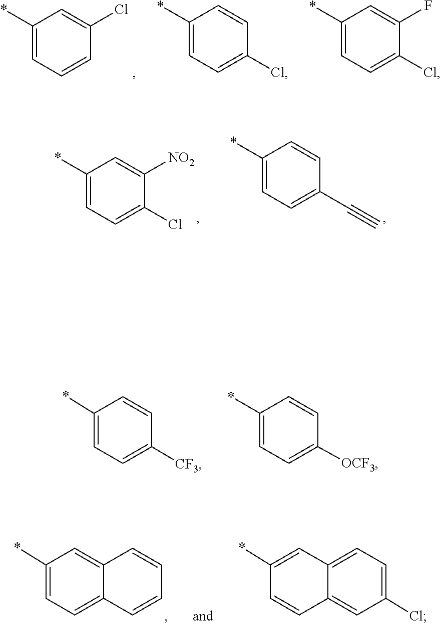

wherein * represents the attachment point to the remainder of the molecule. In some embodiments, A⁸ is

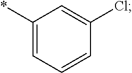 [SMILES: *c1cccc(Cl)c1]

wherein * represents the attachment point to the remainder of the molecule. In some embodiments, A⁸ is

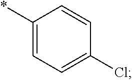 [SMILES: *c1ccc(Cl)cc1]

wherein * represents the attachment point to the remainder of the molecule. In some embodiments, A⁸ is

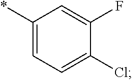 [SMILES: *c1ccc(Cl)c(F)c1]

wherein * represents the attachment point to the remainder of the molecule. In some embodiments, A⁸ is

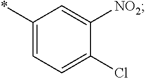 [SMILES: *c1ccc(Cl)c([N+](=O)[O-])c1]

wherein * represents the attachment point to the remainder of the molecule. In some embodiments, A⁸ is

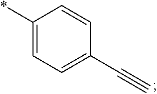 [SMILES: *c1ccc(C#C)cc1]

wherein * represents the attachment point to the remainder of the molecule. In some embodiments, A⁸ is

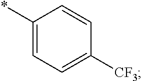 [SMILES: *c1ccc(C(F)(F)F)cc1]

wherein * represents the attachment point to the remainder of the molecule. In some embodiments, A⁸ is

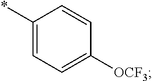 [SMILES: *c1ccc(OC(F)(F)F)cc1]

wherein * represents the attachment point to the remainder of the molecule. In some embodiments, A⁸ is

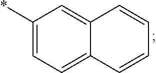 [SMILES: *c1ccc2ccccc2c1]

wherein * represents the attachment point to the remainder of the molecule. In some embodiments, A⁸ is

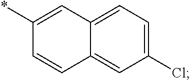 [SMILES: *c1ccc2cc(Cl)ccc2c1]

wherein * represents the attachment point to the remainder of the molecule.

In some embodiments of the compounds of formula (D-1), or the salts thereof, A⁸ is 5-14 membered heteroaryl optionally substituted with 1, 2, 3, 4, 5, 6, 7, 8, or 9 R^{A8} substituents. In some embodiments, A⁸ is selected from the group consisting of

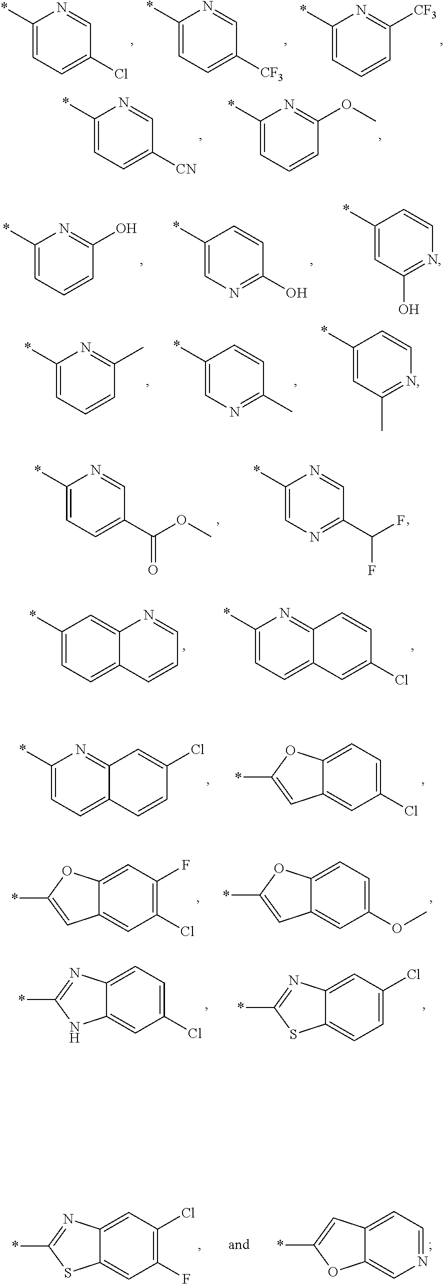

wherein * represents the attachment point to the remainder of the molecule. In some embodiments, A⁸ is selected from the group consisting of

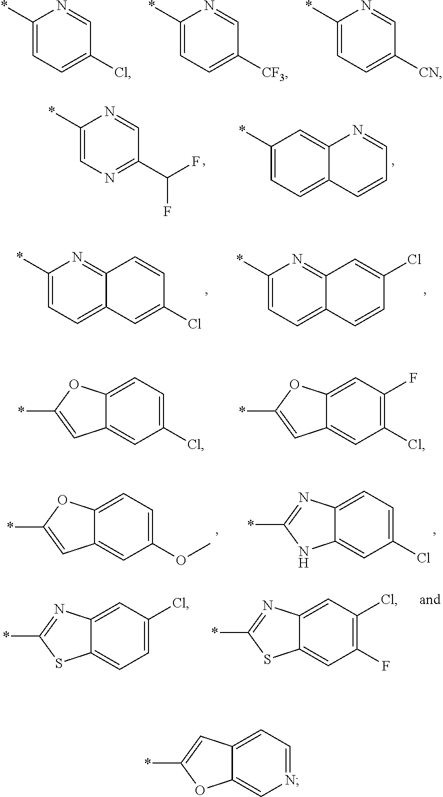

wherein * represents the attachment point to the remainder of the molecule. In some embodiments, A⁸ is

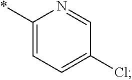 [SMILES: *c1ccc(Cl)cn1]

wherein * represents the attachment point to the remainder of the molecule. In some embodiments, some embodiments, A⁸ is

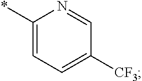 [SMILES: *c1ccc(C(F)(F)F)cn1]

wherein * represents the attachment point to the remainder of the molecule. In some embodiments, A⁸ is

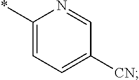 [SMILES: *c1ccc(C#N)cn1]

wherein * represents the attachment point to the remainder of the molecule. In some embodiments, A⁸ is

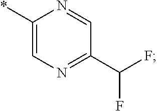 [SMILES: *c1cnc(C(F)F)cn1]

wherein * represents the attachment point to the remainder of the molecule. In some embodiments, A⁸ is

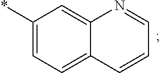 [SMILES: *c1ccc2cccnc2c1]

wherein * represents the attachment point to the remainder of the molecule. In some embodiments, A⁸ is

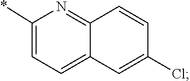 [SMILES: *c1ccc2cc(Cl)ccc2n1]

wherein * represents the attachment point to the remainder of the molecule. In some embodiments, A⁸ is

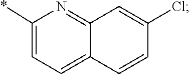 [SMILES: *c1ccc2ccc(Cl)cc2n1]

wherein * represents the attachment point to the remainder of the molecule. In some embodiments, A⁸ is

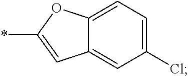 [SMILES: *c1cc2cc(Cl)ccc2o1]

wherein * represents the attachment point to the remainder of the molecule. In some embodiments, A⁸ is

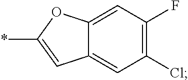 [SMILES: *c1cc2cc(Cl)c(F)cc2o1]

wherein * represents the attachment point to the remainder of the molecule. In some embodiments, A⁸ is

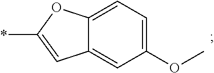 [SMILES: *c1cc2cc(OC)ccc2o1]

wherein * represents the attachment point to the remainder of the molecule. In some embodiments, A⁸ is

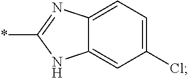 [SMILES: *c1nc2ccc(Cl)cc2[nH]1]

wherein * represents the attachment point to the remainder of the molecule. In some embodiments, A⁸ is

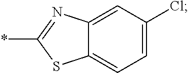 [SMILES: *c1nc2cc(Cl)ccc2s1]

wherein * represents the attachment point to the remainder of the molecule. In some embodiments, A⁸ is

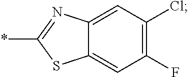 [SMILES: *c1nc2cc(Cl)c(F)cc2s1]

wherein * represents the attachment point to the remainder of the molecule. In some embodiments, A⁸ is

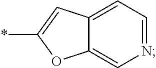 [SMILES: *c1cc2ccncc2o1]

wherein * represents the attachment point to the remainder of the molecule.

In a sixth aspect, provided is a compound of formula (II)

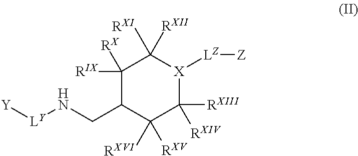

or a pharmaceutically acceptable salt thereof,
wherein:
X is CH or N;
R^{IX}, R^{X}, R^{XI}, R^{XII}, R^{XIII}, R^{IV}, R^{XV}, and R^{XVI}, independently from each other, are selected from the group consisting of hydrogen, C₁-C₆ alkyl, C₁-C₆ haloalkyl, —C(O)OH, —C(O)O(C₁-C₆ alkyl), —C(O)O(C₁-C₆ haloalkyl), and halogen;
or, one of R^{IX}, R^{X}, R^{XI}, R^{XII}, R^{XIII}, R^{IV}, R^{XV}, and R^{XVI}, and another one of R^{IX}, R^{X}, R^{XI}, R^{XII}, R^{XIII}, R^{IV}, R^{XV}, and R^{XVI}, are taken together to form a C₁-C₆ alkylene moiety;
or, two geminal substituents selected from the group consisting of R^{IX}, R^{X}, R^{XI}, R^{XII}, R^{XIII}, R^{IV}, R^{XV}, and R^{XVI} are taken together to form an oxo group;
L^{Y} is selected from the group consisting of

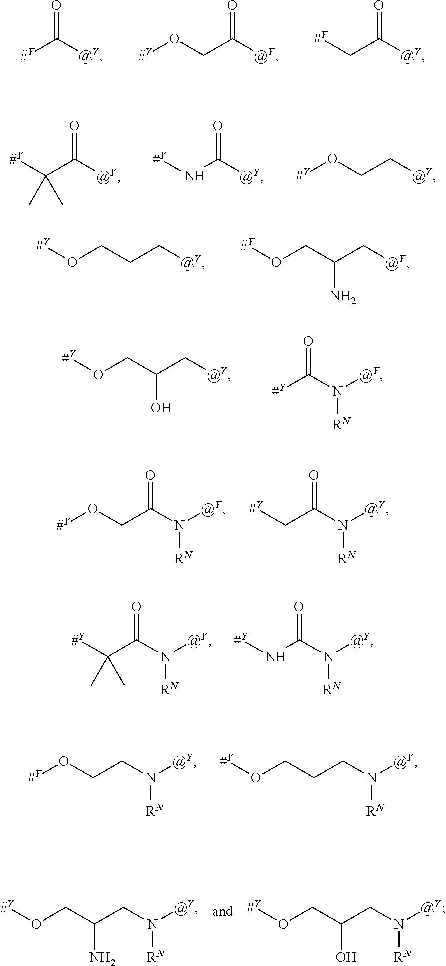

wherein #^{Y} represents the attachment point to Y and @^{Y} represents the attachment point to the remainder of the molecule;
L^{Z} is selected from the group consisting of

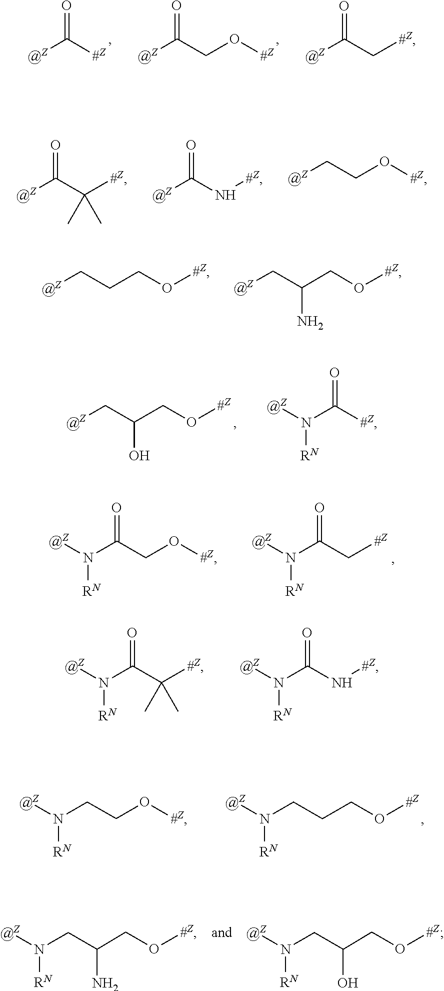

wherein #^{Z} represents the attachment point to Z and @^{Z} represents the attachment point to the remainder of the molecule;
R^{N}, independently at each occurrence, is selected from the group consisting of hydrogen, C₁-C₆ alkyl, and C₁-C₆ haloalkyl,
Y is a substituent of formula (Y-I)

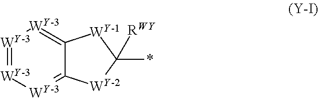

wherein * represents the attachment point to the remainder of the molecule;
   W^{Y-1} is selected from the group consisting of —C(R^{WY-1-1}R^{WY-1-2})—, —N(R^{WY-1-2})—, —C(R^{WY-1-1}R^{WY-1-1})N(R^{WY-1-2})—, —N(R^{WY-1-1})C(R^{WY-1-1}R^{WY-1-2})—, —C(R^{WY-1-1})═N—, —N═C(R^{WY-1-1})—, —O—, —C(R^{WY-1-1}R^{WY-1-1})O—, —OC(R^{WY-1-1}R^{WY-1-2})—, —S, —C(R^{WY-1-1}R^{WY-1-1})S—, —SC(R^{WY-1-1}R^{WY-1-2})—, —C(R^{WY-1-1}R^{WY-1-1})C(R^{WY-1-1}R^{WY-1-2}) and —CR^{WY-1-1}═CR^{WY-1-1}—,
      wherein R^{WY-1-1} is H or R^{Y}, and R^{WY-1-2} is H or R^{Y};
   W^{Y-2} is selected from the group consisting of —C(R^{WY-2-1}R^{WY-2-2})—, —N(R^{WY-2-2})—, —C(R^{WY-2-1}R^{WY-2-1})N(R^{WY-2-2})—, —N(R^{WY-2-1})C(R^{WY-2-1}R^{WY-2-2})—, —C(R^{WY-2-1})═N—, —N═C(R^{WY-2-1})—, —O—, —C(R^{WY-2-1}R^{WY-2-1})O—, —OC(R^{WY-2-1}R^{WY-2-2})—, —S—, —C(R^{WY-2-1}R^{WY-2-1})S—, —SC(R^{WY-2-1}R^{WY-2-2})—, —C(R^{WY-2-1}R^{WY-2-1})C(R^{WY-2-1}R^{WY-2-2}) and —CR^{WY-2-1}═CR^{WY-2-1}—,
      wherein R^{WY-2-1} is H or R^{Y}, and R^{WY-2-2} is H or R^{Y};
   W^{Y-3}, independently at each occurrence, is CR^{WY-3} or N, wherein R^{WY-3} is H or R^{Y};
   R^{WY} is hydrogen or R^{Y}, or R^{WY} and R^{WY-1-2} are taken together to form a double bond between the carbon atom bearing R^{WY} and the atom bearing R^{WY-1-2}, or R^{WY} and R^{WY-2-2} are taken together to form a double bond between the carbon atom bearing R^{WY} and the atom bearing R^{WY-2-2};
   C₆-C₁₄ aryl optionally substituted with 1, 2, 3, 4, 5, 6, 7, 8, or 9 R^{Y} substituents; and
   5-14 membered heteroaryl optionally substituted with 1, 2, 3, 4, 5, 6, 7, 8, or 9 R^{Y} substituents;
      R^{Y}, independently at each occurrence, is selected from the group consisting of halogen, NO₂, C₁-C₆ alkyl, C₂-C₆ alkenyl, C₂-C₆ alkynyl, C₁-C₆ haloalkyl, OH, O(C₁-C₆ alkyl), O(C₁-C₆ haloalkyl), SH, S(C₁-C₆ alkyl), S(C₁-C₆ haloalkyl), NH₂, NH(C₁-C₆ alkyl), NH(C₁-C₆ haloalkyl), N(C₁-C₆ alkyl)₂, N(C₁-C₆ haloalkyl)₂, NR^{a}R^{b}, CN, C(O)OH, C(O)O(C₁-C₆ alkyl), C(O)O(C₁-C₆ haloalkyl), C(O)NH₂, C(O)NH(C₁-C₆ alkyl), C(O)NH(C₁-C₆ haloalkyl), C(O)N(C₁-C₆ alkyl)₂, C(O)N(C₁-C₆ haloalkyl)₂, C(O)NR^{a}R^{b}, S(O)₂OH, S(O)₂O(C₁-C₆ alkyl), S(O)₂O(C₁-C₆ haloalkyl), S(O)₂NH₂, S(O)₂NH(C₁-C₆ alkyl), S(O)₂NH(C₁-C₆ haloalkyl), S(O)₂N(C₁-C₆ alkyl)₂, S(O)₂N(C₁-C₆ haloalkyl)₂, S(O)₂NR^{a}R^{b}, OC(O)H, OC(O)(C₁-C₆ alkyl), OC(O)(C₁-C₆ haloalkyl), N(H)C(O)H, N(H)C(O)(C₁-C₆ alkyl), N(H)C(O)(C₁-C₆ haloalkyl), N(C₁-C₆ alkyl)C(O)H, N(C₁-C₆ alkyl)C(O)(C₁-C₆ alkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ alkyl), OS(O)₂(C₁-C₆ haloalkyl), N(H)S(O)₂(C₁-C₆ alkyl), N(H)S(O)₂(C₁-C₆ haloalkyl), N(C₁-C₆ alkyl)S(O)₂(C₁-C₆ alkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl); wherein R^{a} and R^{b} are taken together with the nitrogen atom to which they are attached to form a 3-10 membered heterocycle;
         and
Z is selected from the group consisting of:
   a substituent of formula (Z-I)

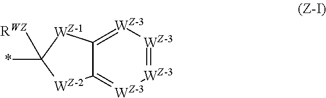

wherein * represents the attachment point to the remainder of the molecule;
   W^{Z-1} is selected from the group consisting of —C(R^{WZ-1}R^{WZ-1-2})—, —N(R^{WZ-1-2})—, —C(R^{WZ-1-1}R^{WZ-1-2})N(R^{WZ-1-2})—, —N(R^{WZ-1-1})C(R^{WZ-1-1}R^{WZ-1-2})—, —C(R^{WZ-1-1})═N—, —N═C(R^{WZ-1-1})—, —O—, —C(R^{WZ-1-1}R^{WZ-1-1})O—, —OC(R^{WZ-1-1}R^{WZ-1-2})—, —S—, —C(R^{WZ-1-1}R^{WZ-1-1})S—, —SC(R^{WZ-1-1}R^{WZ-1-2})—, —C(R^{WZ-1-1}R^{WZ-1-1})C(R^{WZ-1-1}R^{WZ-1-2})—, and —CR^{WZ-1-1}═CR^{WZ-1-1},
      wherein R^{WZ-1-1} is H or R^{Z}, and R^{WZ-1-2} is H or R^{Z};
   W^{Z-2} is selected from the group consisting of —C(R^{WZ-2-1}R^{WZ-2-2})—, —N(R^{WZ-2-2})—, —C(R^{WZ-2-1}R^{WZ-2-1})N(R^{WZ-2-2})—, —N(R^{WZ-2-1})C(R^{WZ-2-1}R^{WZ-2-2})—, —C(R^{WZ-2-1})═N—, —N═C(R^{WZ-2-1})—, —O—, —C(R^{WZ-2-1}R^{WZ-2-1})O—, —OC(R^{WZ-2-1}R^{WZ-2-2})—, —S—, —C(R^{WZ-2-1}R^{WZ-2-1})S—, —SC(R^{WZ-2-1}R^{WZ-2-2})—, —C(R^{WZ-2-1}R^{WZ-2-1})C(R^{WZ-2-1}R^{WZ-2-2})—, and —CR^{WZ-2-1}═CR^{WZ-2-1},
      wherein R^{WZ-2-1} is H or R^{Z}, and R^{WZ-2-2} is H or R^{Z};
   W^{Z-3}, independently at each occurrence, is CR^{WZ-3} or N, wherein R^{WZ-3} is H or R^{Z};
   R^{WZ} is hydrogen or R^{Z}, or R^{WZ} and R^{WZ-1-2} are taken together to form a double bond between the carbon atom bearing R^{WZ} and the atom bearing R^{WZ-1-2}, or R^{WZ} and R^{WZ-2-2} are taken together to form a double bond between the carbon atom bearing R^{WZ} and the atom bearing R^{WZ-2-2};
C₆-C₁₄ aryl optionally substituted with 1, 2, 3, 4, 5, 6, 7, 8, or 9 R^{Z} substituents; and 5-14 membered heteroaryl optionally substituted with 1, 2, 3, 4, 5, 6, 7, 8, or 9 R^{Z} substituents;
   R^{Z}, independently at each occurrence, is selected from the group consisting of halogen, NO₂, C₁-C₆ alkyl, C₂-C₆ alkenyl, C₂-C₆ alkynyl, C₁-C₆ haloalkyl, OH, O(C₁-C₆ alkyl), O(C₁-C₆ haloalkyl), SH, S(C₁-C₆ alkyl), S(C₁-C₆ haloalkyl), NH₂, NH(C₁-C₆ alkyl), NH(C₁-C₆ haloalkyl), N(C₁-C₆ alkyl)₂, N(C₁-C₆ haloalkyl)₂, NR^{a}R^{b}, CN, C(O)OH, C(O)O(C₁-C₆ alkyl), C(O)O(C₁-C₆ haloalkyl), C(O)NH₂, C(O)NH(C₁-C₆ alkyl), C(O)NH(C₁-C₆ haloalkyl), C(O)N(C₁-C₆ alkyl)₂, C(O)N(C₁-C₆ haloalkyl)₂, C(O)NR^{a}R^{b}, S(O)₂OH, S(O)₂O(C₁-C₆ alkyl), S(O)₂O(C₁-C₆ haloalkyl), S(O)₂NH₂, S(O)₂NH(C₁-C₆ alkyl), S(O)₂NH(C₁-C₆ haloalkyl), S(O)₂N(C₁-C₆ alkyl)₂, S(O)₂N(C₁-C₆ haloalkyl)₂, S(O)₂NR^{a}R^{b}, OC(O)H, OC(O)(C₁-C₆ alkyl), OC(O)(C₁-C₆ haloalkyl), N(H)C(O)H, N(H)C(O)(C₁-C₆ alkyl), N(H)C(O)(C₁-C₆ haloalkyl), N(C₁-C₆ alkyl)C(O)H, N(C₁-C₆ alkyl)C(O)(C₁-C₆ alkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ alkyl), OS(O)₂(C₁-C₆ haloalkyl), N(H)S(O)₂(C₁-C₆ alkyl), N(H)S(O)₂(C₁-C₆ haloalkyl), N(C₁-C₆ alkyl)S(O)₂(C₁-C₆ alkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl); wherein R^{a} and R^{b} are taken together with the nitrogen atom to which they are attached to form a 3-10 membered heterocycle;
      provided that when L^{Y} is

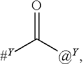

### Y is (Y-I);

when L^{Y} is

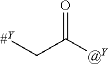

### and L^{Z} is

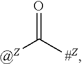

then Y is (Y-I) substituted by 1, 2, 3, 4, 5, 6, 7, 8, or 9 R^{Y} substituents or Z is (Z-I) substituted by 2, 3, 4, 5, 6, 7, 8, or 9 R^{Z} substituents; and
when L^{Y} is

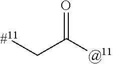

### and L^{Z} is

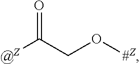

then Y is substituted by 1, 2, 3, 4, 5, 6, 7, 8, or 9 R^{Y} substituents.

In some embodiments of the compounds of formula (II), or the salts thereof, X is CH or N. In some embodiments, X is CH. In some embodiments, X is N.

In some embodiments of the compounds of formula (II), or the salts thereof, R^{IX}, R^{X}, R^{XI}, R^{XII}, R^{XIII}, R^{XIV}, R^{XV}, and R^{XVI} are each hydrogen. In some embodiments, R^{XIII} and R^{XVI} are taken together to form a C₁-C₆ alkylene moiety. In some embodiments, R^{XII} and R^{XVI} are taken together to form a moiety selected from methylene, ethylene, and propylene. In some embodiments, R^{XII} and R^{XVI} are taken together to form a methylene moiety. In some embodiments, R^{XII} and R^{XVI} are taken together to form an ethylene moiety. In some embodiments, R^{IX}, R^{X}, R^{XI}, R^{XIII}, R^{XVI}, and R^{XV} are each hydrogen, and R^{XII} and R^{XVI} are taken together to form a C₁-C₆ alkylene moiety. In some embodiments, R^{IX}, R^{X}, R^{XI}, R^{XIII}, R^{XIV}, and R^{XV} are each hydrogen, and R^{XII} and R^{XVI} are taken together to form a moiety selected from methylene, ethylene, and propylene. In some embodiments, R^{IX}, R^{X}, R^{XI}, R^{XIII}, R^{XIV}, and R^{XV} are each hydrogen, and R^{XII} and R^{XVI} are taken together to form a methylene moiety. In some embodiments, R^{IX}, R^{X}, R^{XI}, R^{XIII}R^{XIV}, and R^{XV} are each hydrogen, and R^{XII} and Rxv are taken together to form an ethylene moiety.

In some embodiments of the compounds of formula (II), or the salts thereof, L^{Y} is selected from the group consisting of

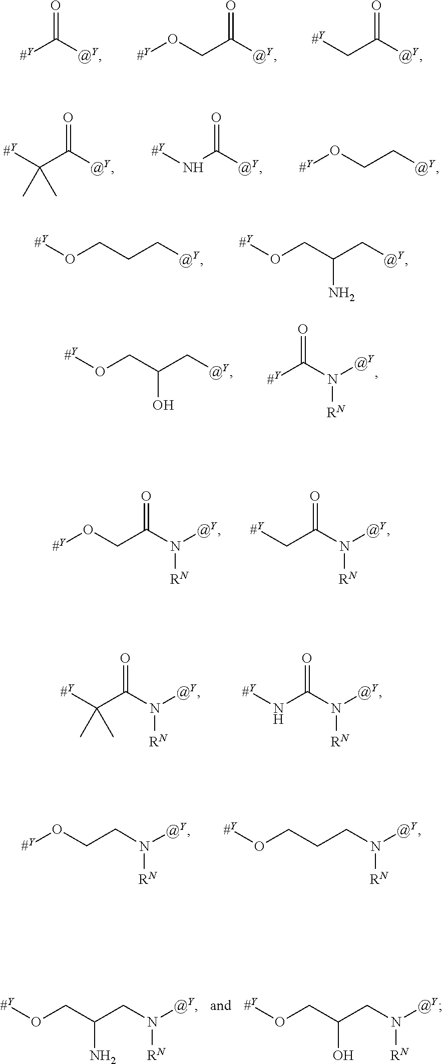

wherein #^{Y} represents the attachment point to Y and @^{Y} represents the attachment point to the remainder of the molecule. In some embodiments, L^{Y} is selected from the group consisting of

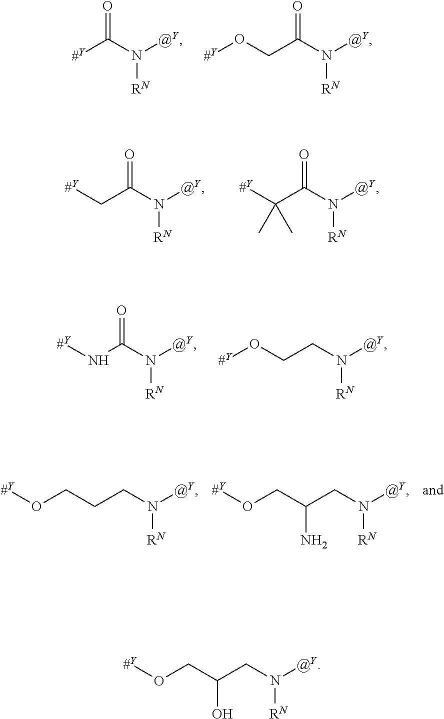

In some embodiments, L^{Y} is selected from the group consisting of

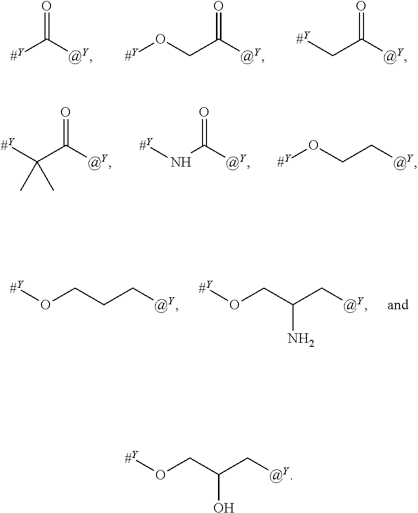

In some embodiments, L^{Y} is

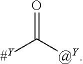

In some embodiments, L^{Y} is

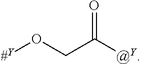

In some embodiments, L^{Y} is

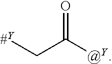

In some embodiments, L^{Y} is

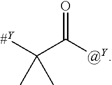

In some embodiments, L^{Y} is

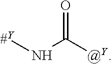

In some embodiments, L^{Y} is

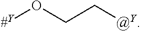

In some embodiments, L^{Y} is

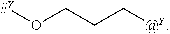

In some embodiments, L^{Y} is

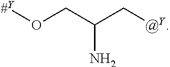

In some embodiments, L^{Y} is

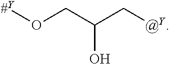

In some embodiments, L^{Y} is

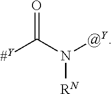

In some embodiments, L^{Y} is

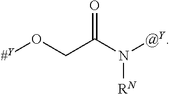

In some embodiments, L^{Y} is

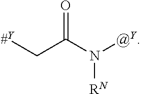

In some embodiments, L^{Y} is

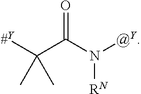

In some embodiments, L^{Y} is

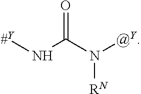

In some embodiments, L^{Y} is

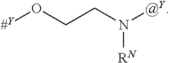

In some embodiments, L^{Y} is

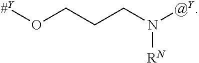

In some embodiments, L^{Y} is

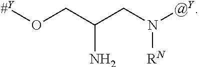

In some embodiments, L^{Y} is

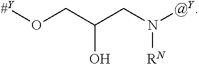

In some embodiments of the compounds of formula (II), or the salts thereof, X is N; and L^{Y} is

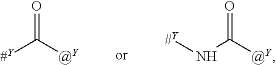

wherein #^{Y} represents the attachment point to Y and @^{Y} represents the attachment point to the remainder of the molecule.

In some embodiments of the compounds of formula (II), or the salts thereof, L^{Z} is selected from the group consisting of

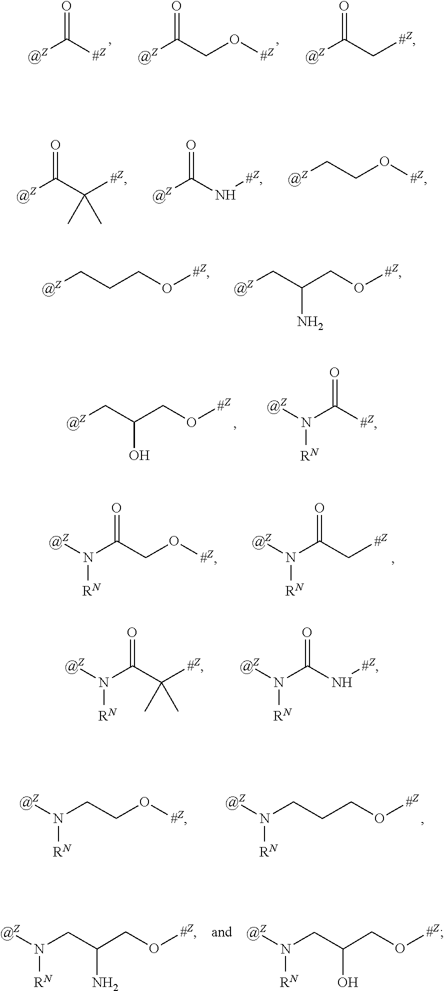

wherein #^{Z} represents the attachment point to Z and @^{Z} represents the attachment point to the remainder of the molecule. In some embodiments, L^{Z} is selected from the group consisting of

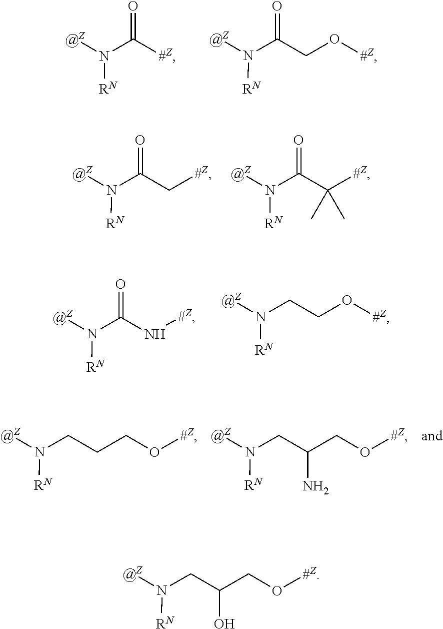

In some embodiments, L^{Z} is selected from the group consisting of

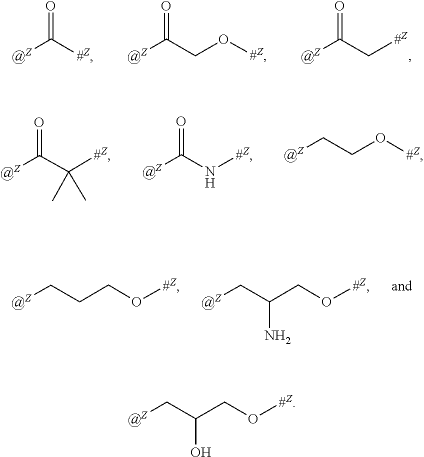

In some embodiments, L^{Z} is

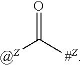

In some embodiments, L^{Z} is

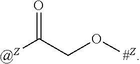

In some embodiments, L^{Z} is

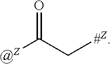

In some embodiments, L^{Z} is

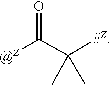

n some embodiments, L^{Z} is

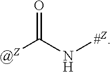

In some embodiments, L^{Z} is

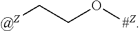

In some embodiments, L^{Z} is

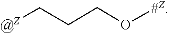

In some embodiments, L^{Z} is

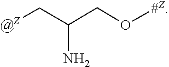

In some embodiments, L^{Z} is

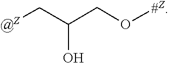

In some embodiments, L^{Z} is

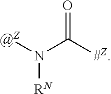

In some embodiments, L^{Z} is

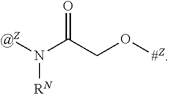

In some embodiments, L^{Z} is

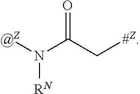

In some embodiments, L^{Z} is

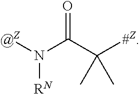

In some embodiments, L^{Z} is

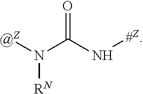

In some embodiments, L^{Z} is

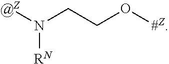

In some embodiments, L^{Z} is

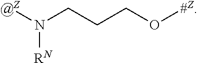

In some embodiments, L^{Z} is

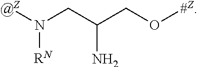

In some embodiments, L^{Z} is

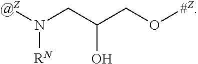

In some embodiments of the compounds of formula (II), or the salts thereof, Y is a substituent of formula (Y-I)

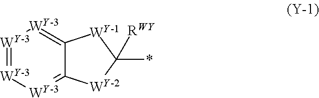

wherein * represents the attachment point to the remainder of the molecule;
W^{Y-1} is selected from the group consisting of —C(R^{WY-1-1}R^{WY-1-2})—, —N(R^{WY-1-2})—, —C(R^{WY-1-1}R^{WY-1-1})N(R^{WY-1-2})—, —N(R^{WY-1-1})C(R^{WY-1-1}R^{WY-1-2})—, —C(R^{WY-1-1})═N—, —N═C(R^{WY-1-1})—, —O—, —C(R^{WY-1-1}R^{WY-1-1})O—, —OC(R^{WY-1-1}R^{WY-1-2})—, —S—, —C(R^{WY-1-1}R^{WY-1-1})S—, —SC(R^{WY-1-1}R^{WY-1-2})—, —C(R^{WY-1-1}R^{WY-1-1})C(R^{WY-1-1}R^{WY-1-2})—, and —CR^{WY-1-1}═CR^{WY-1-1}—,

wherein R^{WY-1-1} is H or R^{Y}, and R^{WY-1-2} is H or R^{Y};

W^{Y-2} is selected from the group consisting of —C(R^{WY-2-1}R^{WY-2-2})—, —N(R^{WY-2-2})—, —C(R^{WY-2-1}R^{WY-2-1})N(R^{WY-2-2})—, —N(R^{WY-2-1})C(R^{WY-2-1}R^{WY-2-2})—, —C(R^{WY-2-1})═N—, —N═C(R^{WY-2-1})—, —O—, —C(R^{WY-2-1}R^{WY-2-1})O—, —OC(R^{WY-2-1}R^{WY-2-2})—, —S—, —C(R^{WY-2-1}R^{WY-2-1})S—, —SC(R^{WY-2-1}R^{WY-2-2})—, —C(R^{WY-2-1}R^{WY-2-1})C(R^{WY-2-1}R^{WY-2-2})—, and —CR^{WY-2-1}═CR^{WY-2-1}—,

wherein R^{WY-2-1} is H or R^{Y}, and R^{WY-2-2} is H or R^{Y};

W^{Y-3}, independently at each occurrence, is CR^{WY-3} or N, wherein R^{WY-3} is H or R^{Y}; R^{WY} is hydrogen or R^{Y}, or R^{WY} and R^{WY-1-2} are taken together to form a double bond between the carbon atom bearing R^{WY} and the atom bearing R^{WY-1-2}, or R^{WY} and R^{WY-2-2} are taken together to form a double bond between the carbon atom bearing R^{Y} and the atom bearing R^{WY-2-2}.

In some embodiments, (Y-I) is selected from the group consisting of

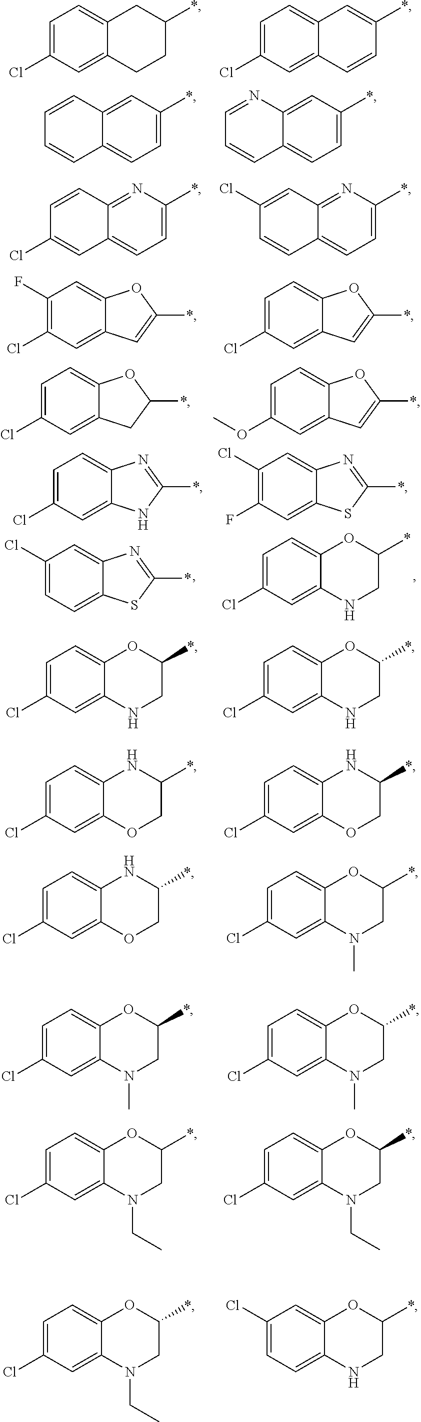

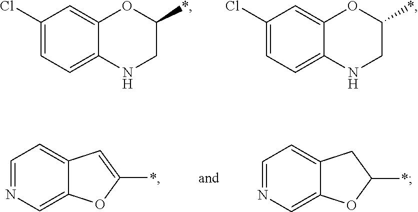

wherein * represents the attachment point to the remainder of the molecule. In some embodiments, (Y-I) is selected from the group consisting of

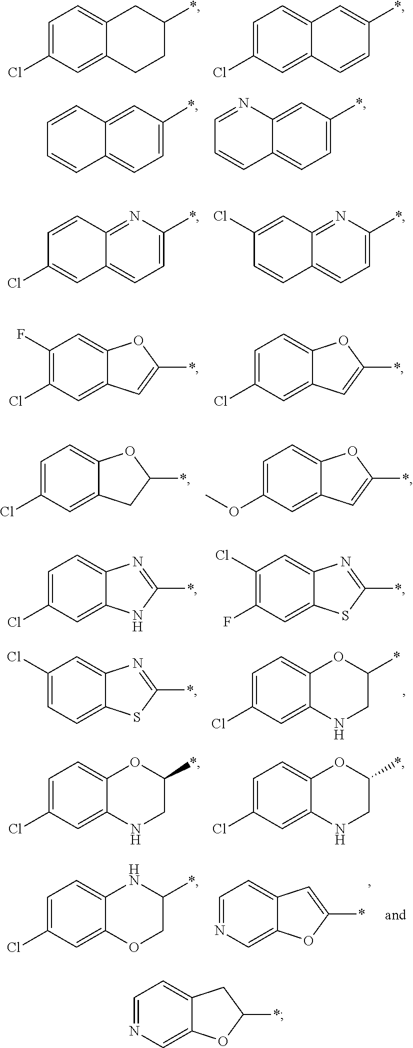

wherein * represents the attachment point to the remainder of the molecule. In some embodiments, (Y-I) is

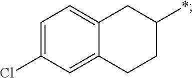

wherein * represents the attachment point to the remainder of the molecule. In some embodiments, (Y-I) is

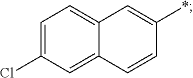

wherein * represents the attachment point to the remainder of the molecule. In some embodiments, (Y-I) is

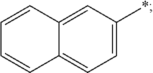

wherein * represents the attachment point to the remainder of the molecule. In some embodiments, (Y-I) is

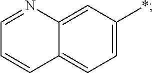

wherein * represents the attachment point to the remainder of the molecule. In some embodiments (Y-I) is

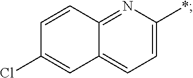

wherein * represents the attachment point to the remainder of the molecule. In some embodiments, (Y-I) is

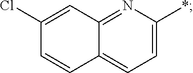

wherein * represents the attachment point to the remainder of the molecule. In some embodiments, (Y-I) is

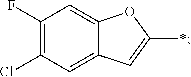

wherein * represents the attachment point to the remainder of the molecule. In some embodiments, (Y-I) is

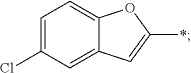

wherein * represents the attachment point to the remainder of the molecule. In some embodiments, (Y-I) is

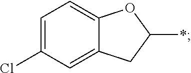

wherein * represents the attachment point to the remainder of the molecule. In some embodiments, (Y-I) is

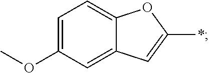

wherein * represents the attachment point to the remainder of the molecule. In some embodiments, (Y-I) is

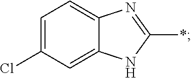

wherein * represents the attachment point to the remainder of the molecule. In some embodiments, (Y-I) is

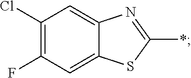

wherein * represents the attachment point to the remainder of the molecule. In some embodiments, (Y-I) is

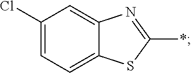

wherein * represents the attachment point to the remainder of the molecule. In some embodiments, (Y-I) is

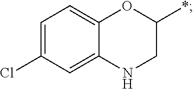

wherein * represents the attachment point to the remainder of the molecule. In some embodiments, (Y-I) is

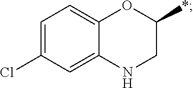

wherein * represents the attachment point to the remainder of the molecule. In some embodiments, (Y-I) is

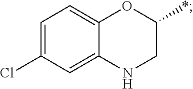

wherein * represents the attachment point to the remainder of the molecule. In some embodiments, (Y-I) is

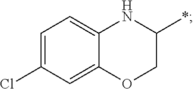

wherein * represents the attachment point to the remainder of the molecule. In some embodiments, (Y-I) is

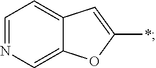

wherein * represents the attachment point to the remainder of the molecule. In some embodiments, (Y-I) is

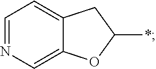

wherein * represents the attachment point to the remainder of the molecule.

In some embodiments of the compounds of formula (II), or the salts thereof, Y is C₆-C₁₄ aryl optionally substituted with 1, 2, 3, 4, 5, 6, 7, 8, or 9 R^{Y} substituents. In some embodiments, Y is selected from the group consisting of

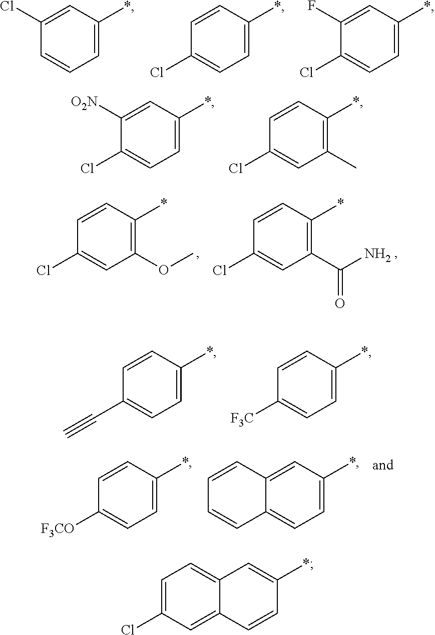

wherein * represents the attachment point to the remainder of the molecule. In some embodiments Y is selected from the group consisting of

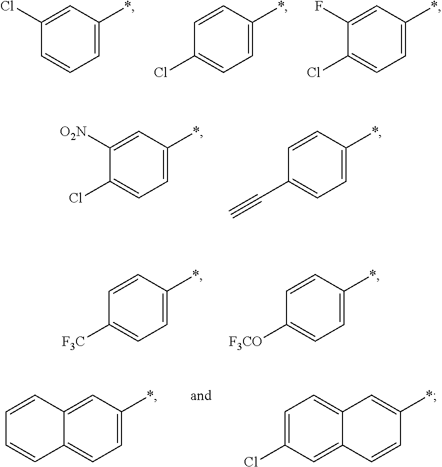

wherein * represents the attachment point to the remainder of the molecule. In some embodiments, Y is

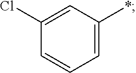

wherein * represents the attachment point to the remainder of the molecule. In some embodiments, Y is

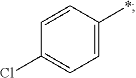

wherein * represents the attachment point to the remainder of the molecule. In some embodiments, Y is

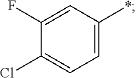

wherein * represents the attachment point to the remainder of the molecule. In some embodiments, Y is

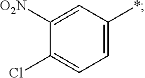

wherein * represents the attachment point to the remainder of the molecule. In some embodiments, Y is

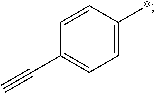

wherein * represents the attachment point to the remainder of the molecule. In some embodiments, Y is

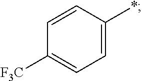

wherein * represents the attachment point to the remainder of the molecule. In some embodiments, Y is

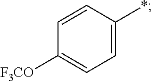

wherein * represents the attachment point to the remainder of the molecule. In some embodiments, Y is

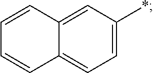

wherein * represents the attachment point to the remainder of the molecule. In some embodiments, Y is

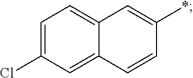

wherein * represents the attachment point to the remainder of the molecule.

In some embodiments of the compounds of formula (II), or the salts thereof, Y is 5-14 membered heteroaryl optionally substituted with 1, 2, 3, 4, 5, 6, 7, 8, or 9 R^{Y} substituents. In some embodiments, Y is selected from the group consisting of

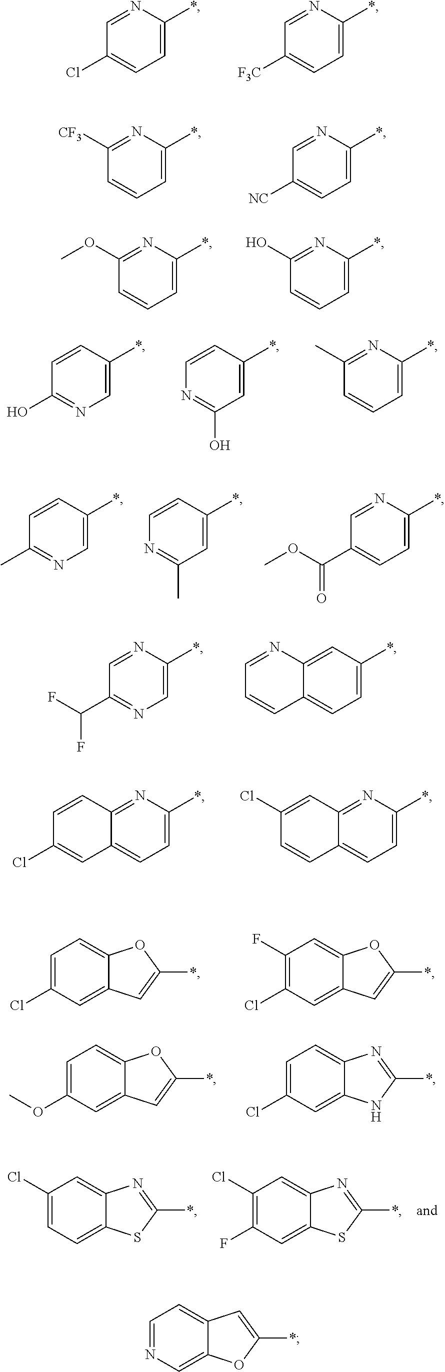

wherein * represents the attachment point to the remainder of the molecule. In some embodiments, Y is selected from the group consisting of

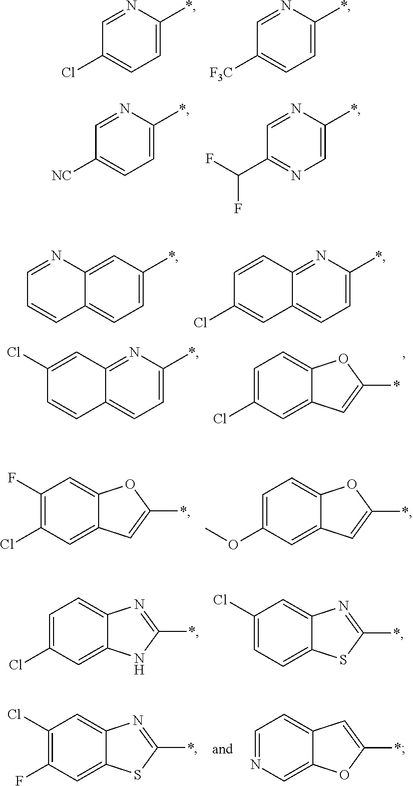

wherein * represents the attachment point to the remainder of the molecule. In some embodiments, Y is

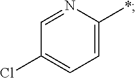

wherein * represents the attachment point to the remainder of the molecule. In some embodiments, Y is

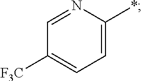

wherein * represents the attachment point to the remainder of the molecule. In some embodiments, Y is

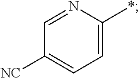

wherein * represents the attachment point to the remainder of the molecule. In some embodiments, Y is

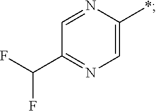

wherein * represents the attachment point to the remainder of the molecule. In some embodiments, Y is

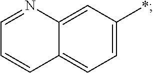

wherein * represents the attachment point to the remainder of the molecule. In some embodiments, Y is

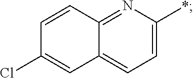

wherein * represents the attachment point to the remainder of the molecule. In some embodiments, Y is

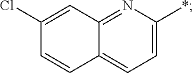

wherein * represents the attachment point to the remainder of the molecule. In some embodiments, Y is

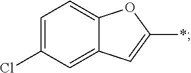

wherein * represents the attachment point to the remainder of the molecule. In some embodiments, Y is

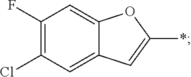

wherein * represents the attachment point to the remainder of the molecule. In some embodiments, Y is

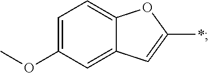

wherein * represents the attachment point to the remainder of the molecule. In some embodiments, Y is

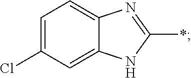

wherein * represents the attachment point to the remainder of the molecule. In some embodiments, Y is

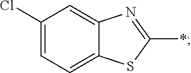

wherein * represents the attachment point to the remainder of the molecule. In some embodiments, Y is

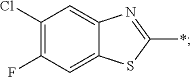

wherein * represents the attachment point to the remainder of the molecule. In some embodiments, Y is

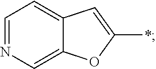

wherein * represents the attachment point to the remainder of the molecule.

In some embodiments of the compounds of formula (II), Z is a substituent of formula (Z-I)

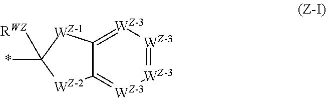

wherein * represents the attachment point to the remainder of the molecule;
W^{Z-1} is selected from the group consisting of —C(R^{WZ-1-1}R^{WZ-1-2})—, —N(R^{WZ-1-2})—, —C(R^{WZ-1-1}R^{WZ-1-2})N(R^{WZ-1-2})—, —N(R^{WZ-1-1})C(R^{WZ-1-1}R^{WZ-1-2})—, —C(R^{WZ-1-1})═N—, —N═C(R^{WZ-1-1})—, —O—, —C(R^{WZ-1-1}R^{WZ-1-1})O—, —OC(R^{WZ-1-1}R^{WZ-1-2})—, —S—, —C(R^{WZ-1-1}R^{WZ-1-1})S—, —SC(R^{WZ-1-1}R^{WZ-1-2})—, —C(R^{WZ-1-1}R^{WZ-1-1})C(R^{WZ-1-1}R^{WZ-1-2})—, and —CR^{WZ-1-1}═CR^{WZ-1-1}—,

wherein R^{WZ-1-1} is H or R^{Z}, and R^{WZ-1-2} is H or R^{Z};

W^{Z-2} is selected from the group consisting of —C(R^{WZ-2-1}R^{WZ-2-2})—, —N(R^{WZ-2-2})—, —C(R^{WZ-2-1}R^{WZ-2-1})N(R^{WZ-2-2})—, —N(R^{WZ-2-1})C(R^{WZ-2-1}R^{WZ-2-2})—, —C(R^{WZ-2-1})═N—, —N═C(R^{WZ-2-1})—, —O—, —C(R^{WZ-2-1}R^{WZ-2-1})O—, —OC(R^{WZ-2-1}R^{WZ-2-2})—, —S—, —C(R^{WZ-2-1}R^{WZ-2-1})S—, —SC(R^{WZ-2-1}R^{WZ-2-2})—, —C(R^{WZ-2-1}R^{WZ-2-1})C(R^{WZ-2-1}R^{WZ-2-2})—, and —CR^{WZ-2-1}═CR^{WZ-2-1}—,

wherein R^{WZ-2-1} is H or R^{Z}, and R^{WZ-2-2} is H or R^{Z};

W^{Z-3}, independently at each occurrence, is CR^{WZ-3} or N, wherein R^{WZ-3} is H or R^{Z};
R^{WZ} is hydrogen or R^{Z}, or R^{WZ} and R^{WZ-1-2} are taken together to form a double bond between the carbon atom bearing R^{WZ} and the atom bearing R^{WZ-1-2}, or R^{WZ} and R^{WZ-2-2} are taken together to form a double bond between the carbon atom bearing R^{WZ} and the atom bearing R^{WZ-2-2}.

In some embodiments, (Z-I) is selected from the group consisting of

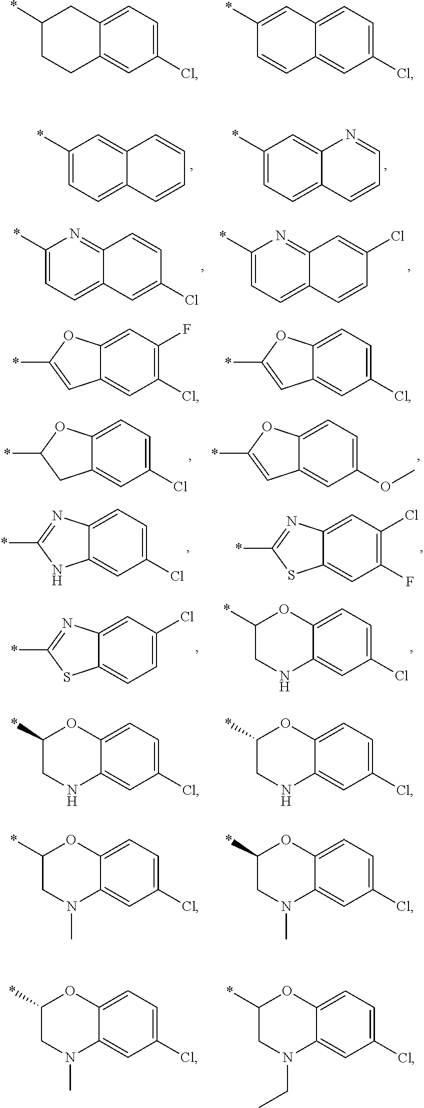

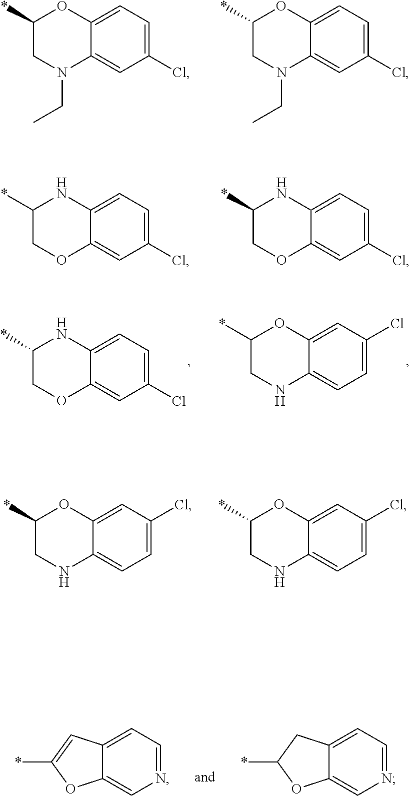

wherein * represents the attachment point to the remainder of the molecule. In some embodiments, (Z-I) is selected from the group consisting of

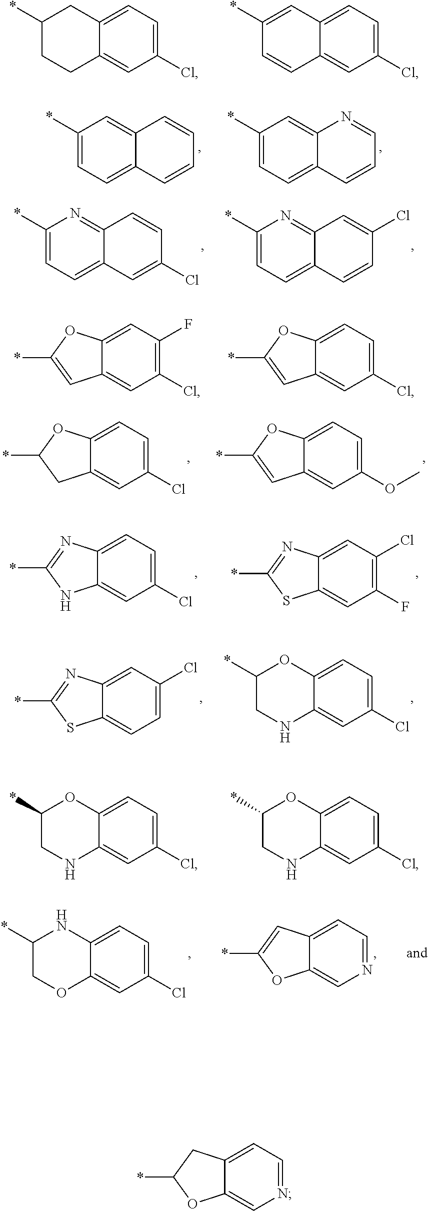

wherein * represents the attachment point to the remainder of the molecule. In some embodiments, (Z-I) is

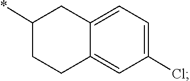 [SMILES: *C1CCc2cc(Cl)ccc2C1]

wherein * represents the attachment point to the remainder of the molecule. In some embodiments, (Z-I) is

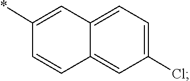 [SMILES: *c1ccc2cc(Cl)ccc2c1]

wherein * represents the attachment point to the remainder of the molecule. In some embodiments, (Z-I) is

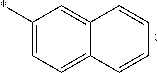 [SMILES: *c1ccc2ccccc2c1]

wherein * represents the attachment point to the remainder of the molecule. In some embodiments, (Z-I) is

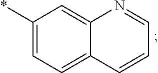 [SMILES: *c1ccc2cccnc2c1]

wherein * represents the attachment point to the remainder of the molecule. In some embodiments, (Z-I) is

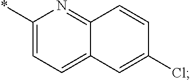 [SMILES: *c1ccc2cc(Cl)ccc2n1]

wherein * represents the attachment point to the remainder of the molecule. In some embodiments, (Z-I) is

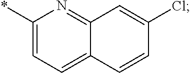 [SMILES: *c1ccc2ccc(Cl)cc2n1]

wherein * represents the attachment point to the remainder of the molecule. In some embodiments, (Z-I) is

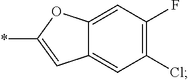 [SMILES: *c1cc2cc(Cl)c(F)cc2o1]

wherein * represents the attachment point to the remainder of the molecule. In some embodiments, (Z-I) is

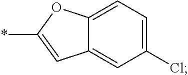 [SMILES: *c1cc2cc(Cl)ccc2o1]

wherein * represents the attachment point to the remainder of the molecule. In some embodiments, (Z-I) is

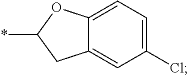 [SMILES: *C1Cc2cc(Cl)ccc2O1]

wherein * represents the attachment point to the remainder of the molecule. In some embodiments, (Z-I) is

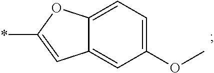 [SMILES: *c1cc2cc(OC)ccc2o1]

wherein * represents the attachment point to the remainder of the molecule. In some embodiments, (Z-I) is

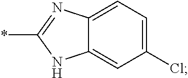 [SMILES: *c1nc2ccc(Cl)cc2[nH]1]

wherein * represents the attachment point to the remainder of the molecule. In some embodiments, (Z-I) is

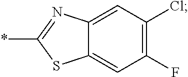 [SMILES: *c1nc2cc(Cl)c(F)cc2s1]

wherein * represents the attachment point to the remainder of the molecule. In some embodiments, (Z-I) is

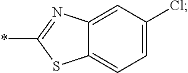 [SMILES: *c1nc2cc(Cl)ccc2s1]

wherein * represents the attachment point to the remainder of the molecule. In some embodiments, (Z-I) is

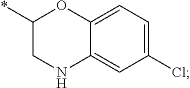 [SMILES: *C1CNc2cc(Cl)ccc2O1]

wherein * represents the attachment point to the remainder of the molecule. In some embodiments, (Z-I) is

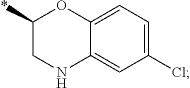 [SMILES: *[C@@H]1CNc2cc(Cl)ccc2O1]

wherein * represents the attachment point to the remainder of the molecule. In some embodiments, (Z-I) is

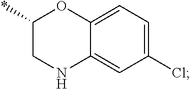 [SMILES: *[C@H]1CNc2cc(Cl)ccc2O1]

wherein * represents the attachment point to the remainder of the molecule. In some embodiments, (Z-I) is

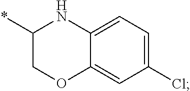 [SMILES: *C1COc2cc(Cl)ccc2N1]

wherein * represents the attachment point to the remainder of the molecule. In some embodiments, (Z-I) is

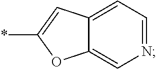 [SMILES: *c1cc2ccncc2o1]

wherein * represents the attachment point to the remainder of the molecule. In some embodiments, (Z-I) is

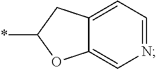 [SMILES: *C1Cc2ccncc2O1]

wherein * represents the attachment point to the remainder of the molecule.

In some embodiments of the compounds of formula (II), or the salts thereof, Z is C₆-C₁₄ aryl optionally substituted with 1, 2, 3, 4, 5, 6, 7, 8, or 9 R^{Z} substituents. In some embodiments, Z is selected from the group consisting of

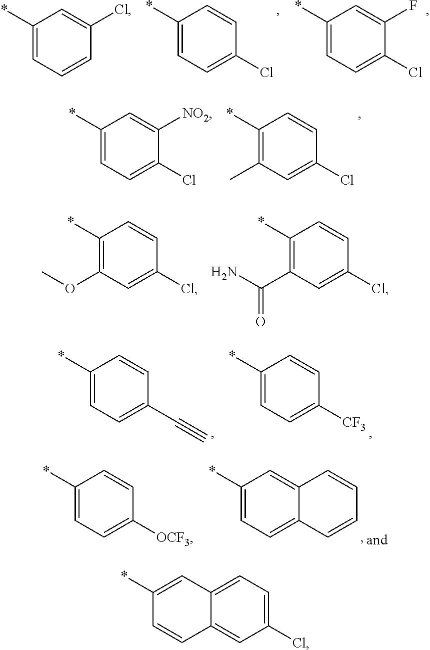

wherein * represents the attachment point to the remainder of the molecule. In some embodiments, Z is selected from the group consisting of

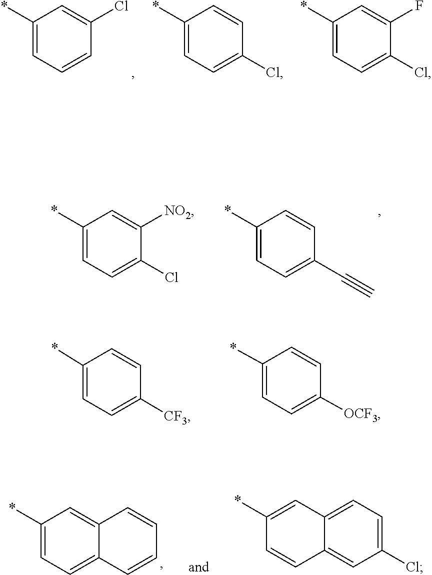

wherein * represents the attachment point to the remainder of the molecule. In some embodiments, Z is

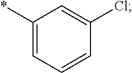 [SMILES: *c1cccc(Cl)c1]

wherein * represents the attachment point to the remainder of the molecule. In some embodiments, Z is

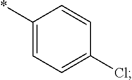 [SMILES: *c1ccc(Cl)cc1]

wherein * represents the attachment point to the remainder of the molecule. In some embodiments, Z is

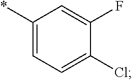 [SMILES: *c1ccc(Cl)c(F)c1]

wherein * represents the attachment point to the remainder of the molecule. In some embodiments, Z is

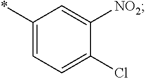 [SMILES: *c1ccc(Cl)c([N+](=O)[O-])c1]

wherein * represents the attachment point to the remainder of the molecule. In some embodiments, Z is

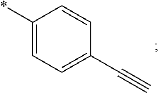 [SMILES: *c1ccc(C#C)cc1]

wherein * represents the attachment point to the remainder of the molecule. In some embodiments, Z is

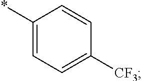 [SMILES: *c1ccc(C(F)(F)F)cc1]

wherein * represents the attachment point to the remainder of the molecule. In some embodiments, Z is

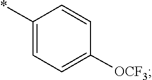 [SMILES: *c1ccc(OC(F)(F)F)cc1]

wherein * represents the attachment point to the remainder of the molecule. In some embodiments, Z is

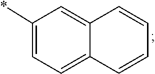 [SMILES: *c1ccc2ccccc2c1]

wherein * represents the attachment point to the remainder of the molecule. In some embodiments, Z is

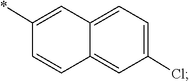 [SMILES: *c1ccc2cc(Cl)ccc2c1]

wherein * represents the attachment point to the remainder of the molecule.

In some embodiments of the compounds of formula (II), or the salts thereof, Z is 5-14 membered heteroaryl optionally substituted with 1, 2, 3, 4, 5, 6, 7, 8, or 9 R^{Z} substituents. In some embodiments, Z is selected from the group consisting of

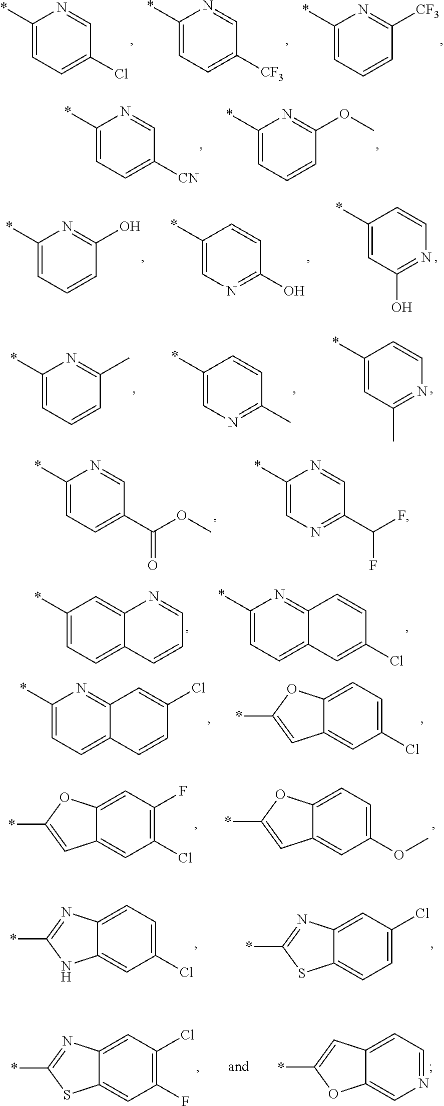

wherein * represents the attachment point to the remainder of the molecule. In some embodiments, Z is selected from the group consisting of

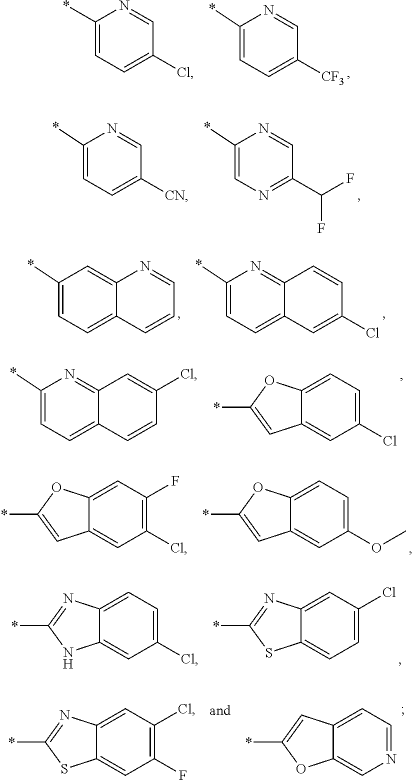

wherein * represents the attachment point to the remainder of the molecule. In some embodiments, Z is

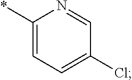 [SMILES: *c1ccc(Cl)cn1]

wherein * represents the attachment point to the remainder of the molecule. In some embodiments, Z is

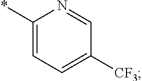 [SMILES: *c1ccc(C(F)(F)F)cn1]

wherein * represents the attachment point to the remainder of the molecule. In some embodiments, Z is

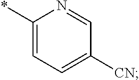 [SMILES: *c1ccc(C#N)cn1]

wherein * represents the attachment point to the remainder of the molecule. In some embodiments, Z is

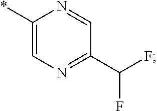 [SMILES: *c1cnc(C(F)F)cn1]

wherein * represents the attachment point to the remainder of the molecule. In some embodiments, Z is

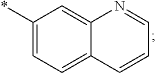 [SMILES: *c1ccc2cccnc2c1]

wherein * represents the attachment point to the remainder of the molecule. In some embodiments, Z is

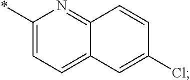 [SMILES: *c1ccc2cc(Cl)ccc2n1]

wherein * represents the attachment point to the remainder of the molecule. In some embodiments, Z is

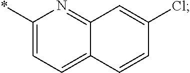 [SMILES: *c1ccc2ccc(Cl)cc2n1]

wherein * represents the attachment point to the remainder of the molecule. In some embodiments, Z is

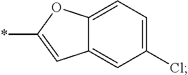 [SMILES: *c1cc2cc(Cl)ccc2o1]

wherein * represents the attachment point to the remainder of the molecule. In some embodiments, Z is

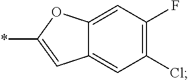 [SMILES: *c1cc2cc(Cl)c(F)cc2o1]

wherein * represents the attachment point to the remainder of the molecule. In some embodiments, Z is

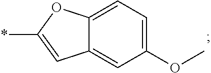 [SMILES: *c1cc2cc(OC)ccc2o1]

wherein * represents the attachment point to the remainder of the molecule. In some embodiments, Z is

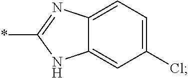 [SMILES: *c1nc2ccc(Cl)cc2[nH]1]

wherein * represents the attachment point to the remainder of the molecule. In some embodiments, Z is

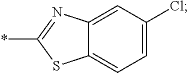 [SMILES: *c1nc2cc(Cl)ccc2s1]

wherein * represents the attachment point to the remainder of the molecule. In some embodiments, Z is

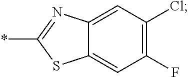 [SMILES: *c1nc2cc(Cl)c(F)cc2s1]

wherein * represents the attachment point to the remainder of the molecule. In some embodiments, Z is

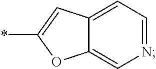 [SMILES: *c1cc2ccncc2o1]

wherein * represents the attachment point to the remainder of the molecule.

In a seventh aspect, provided is a compound of formula (E-1)

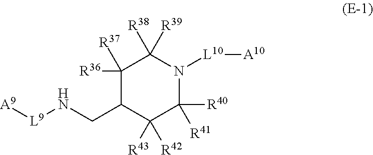

or a pharmaceutically acceptable salt thereof,
wherein:
R³⁶, R³⁷, R³⁸, R³⁹, R⁴⁰, R⁴¹, R⁴², and R⁴³, independently from each other, are selected from the group consisting of hydrogen, C₁-C₆ alkyl, C₁-C₆ haloalkyl, —C(O)OH, —C(O)O(C₁-C₆ alkyl), —C(O)O(C₁-C₆ haloalkyl), and halogen;
or, one of R²⁷, R²⁸, R²⁹, R³⁰, R³¹, R³², R³³, and R³⁴, and another one of R²⁷, R²⁸, R²⁹, R³⁰, R³¹, R³², R³³, and R³⁴, are taken together to form a C₁-C₆ alkylene moiety;
or, two geminal substituents selected from the group consisting of R²⁷, R²⁸, R²⁹, R³⁰, R³¹, R³², R³³, and R³⁴ are taken together to form an oxo group;
L⁹ is selected from the group consisting of a bond,

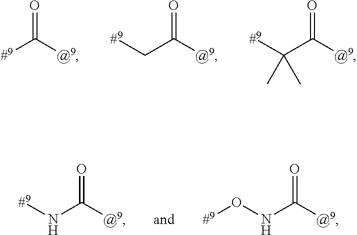

wherein #⁹ represents to attachment point to A⁹ and @⁹ represents the attachment point to the remainder of the molecule;
L¹⁰ is selected from the group consisting of

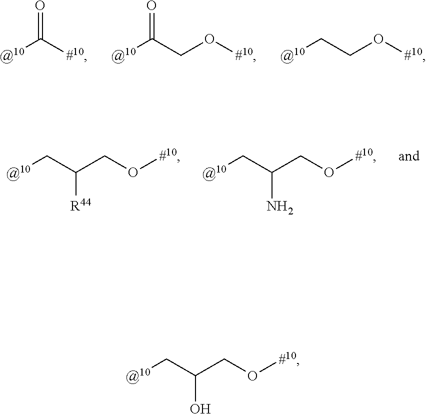

wherein #¹⁰ represents to attachment point to A¹⁰ and @¹⁰ represents the attachment point to the remainder of the molecule;
R⁴⁴ is H, OH, or NH₂;
A⁹ is selected from the group consisting of:
   a substituent of formula (A⁹-1)

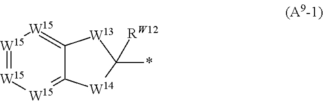

wherein * represents the attachment point to the remainder of the molecule;
   W¹³ is selected from the group consisting of —C(R^{W13-1}R^{W13-2})—, —N(R^{W13-2})—, —C(R^{W13-1}R^{W13-2})N(R^{W13-2})—, —N(R^{W13-1})C(R^{W13-1}R^{W13-2})—, —C(R^{W13-1})═N—, —N═C(R^{W13-1})—, —O—, —C(R^{W13-1}R^{W13-1})O—, —OC(R^{W3-1}R^{W13-2})—, —S—, —C(R^{W13-1}R^{W13-1})S—, —SC(R^{W13-1}R^{W13-2})—, —C(R^{W13-1}R^{W13-1})C(R^{W13-1}R^{W13-2})—, and —CR^{W13-1}═CR^{W13-1}—,
   wherein R^{W13-1} is H or R^{A9}, and R^{W13-2} is H or R^{A9};
   W¹⁴ is selected from the group consisting of —C(R^{W14-1}R^{W14-2})—, —N(R^{W14-2})—, —C(R^{W14-1}R^{W14-1})N(R^{W14-2})—, —N(R^{W14-1})C(R^{W14-1}R^{W14-2})—, —C(R^{W14-1})═N—, —N═C(R^{W14-1})—, —O—, —C(R^{W14-1}R^{W14-1})O—, —OC(R^{W14-1}R^{W14-2})—, —S—, —C(R^{W14-1}R^{W14-1})S—, —SC(R^{W14-1}R^{W14-2})—, —C(R^{W14-1}R^{W14-1})C(R^{W14-1}R^{W14-2})—, and —CR^{W14-1}═CR^{W14-1}—,
   wherein R^{W14-1} is H or R^{A9}, and R^{W14-2} is H or R^{A9};
   W¹⁵, independently at each occurrence, is CR^{W15} or N, wherein R^{W15} is H or R^{A9};
   R^{W12} is hydrogen or R^{A9}, or R^{W12} and R^{W13-2} are taken together to form a double bond between the carbon atom bearing R^{W12} and the atom bearing R^{W13-2}, or R^{W2} and R^{W14-2} are taken together to form a double bond between the carbon atom bearing R^{W12} and the atom bearing R^{W14-2}.
C₆-C₁₄ aryl optionally substituted with 1, 2, 3, 4, 5, 6, 7, 8, or 9 R^{A9} substituents; and
5-14 membered heteroaryl optionally substituted with 1, 2, 3, 4, 5, 6, 7, 8, or 9 R^{A9} substituents;
R^{A9}, independently at each occurrence, is selected from the group consisting of halogen, NO₂, C₁-C₆ alkyl, C₂-C₆ alkenyl, C₂-C₆ alkynyl, C₁-C₆ haloalkyl, OH, O(C₁-C₆ alkyl), O(C₁-C₆ haloalkyl), SH, S(C₁-C₆ alkyl), S(C₁-C₆ haloalkyl), NH₂, NH(C₁-C₆ alkyl), NH(C₁-C₆ haloalkyl), N(C₁-C₆ alkyl)₂, N(C₁-C₆ haloalkyl)₂, NR^{a}R^{b}, CN, C(O)OH, C(O)O(C₁-C₆ alkyl), C(O)O(C₁-C₆ haloalkyl), C(O)NH₂, C(O)NH(C₁-C₆ alkyl), C(O)NH(C₁-C₆ haloalkyl), C(O)N(C₁-C₆ alkyl)₂, C(O)N(C₁-C₆ haloalkyl)₂, C(O)NR^{a}R^{b}, S(O)₂OH, S(O)₂O(C₁-C₆ alkyl), S(O)₂O(C₁-C₆ haloalkyl), S(O)₂NH₂, S(O)₂NH(C₁-C₆ alkyl), S(O)₂NH(C₁-C₆ haloalkyl), S(O)₂N(C₁-C₆ alkyl)₂, S(O)₂N(C₁-C₆ haloalkyl)₂, S(O)₂NR^{a}R^{b}, OC(O)H, OC(O)(C₁-C₆ alkyl), OC(O)(C₁-C₆ haloalkyl), N(H)C(O)H, N(H)C(O)(C₁-C₆ alkyl), N(H)C(O)(C₁-C₆ haloalkyl), N(C₁-C₆ alkyl)C(O)H, N(C₁-C₆ alkyl)C(O)(C₁-C₆ alkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ alkyl), OS(O)₂(C₁-C₆ haloalkyl), N(H)S(O)₂(C₁-C₆ alkyl), N(H)S(O)₂(C₁-C₆ haloalkyl), N(C₁-C₆ alkyl)S(O)₂(C₁-C₆ alkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl); wherein R^{a} and R^{b} are taken together with the nitrogen atom to which they are attached to form a 3-10 membered heterocycle;
   and
A¹⁰ is selected from the group consisting of:
   a substituent of formula (A¹⁰-1)

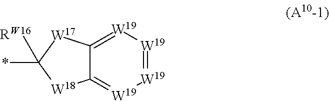

wherein * represents the attachment point to the remainder of the molecule;
   W¹⁷ is selected from the group consisting of —C(R^{W17-1}R^{W17-2})—, —N(R^{W17-2})—, —C(R^{W17-1}R^{W17-2})N(R^{W17-2})—, —N(R^{W17-1})C(R^{W17-1}R^{W17-2})—, —C(R^{W17-1})═N—, —N═C(R^{W17-1})—, —O—, —C(R^{W17-1}R^{W17-1})O—, —OC(R^{W17-1}R^{W17-2})—, —S—, —C(R^{W17-1}R^{W17-1})S—, —SC(R^{W17-1}R^{W17-2})—, —C(R^{W17-1}R^{W17-1})C(R^{W17-1}R^{W17-2})—, and —CR^{W17-1}═CR^{W17-1}—,
   wherein R^{W17-1} is H or R^{A}, and R^{W17-2} is H or R^{A10};
   W¹⁸ is selected from the group consisting of —C(R^{W18-1}R^{W18-2})—, —N(R^{W18-2})—, —C(R^{W18-1}R^{W18-1})N(R^{W18-2})—, —N(R^{W18-1})C(R^{W18-1}R^{W18-2})—, —C(R^{W18-1})═N—, —N═C(R^{W18-1})—, —O—, —C(R^{W18-1}R^{W18-1})O—, —OC(R^{W18-1}R^{W18-2})—, —S—, —C(R^{W18-1}R^{W18-1})S—, —SC(R^{W18-1}R^{W18-2})—, —C(R^{W18-1}R^{W18-1})C(R^{W18-1}R^{W18-2})—, and —CR^{W18-1}═CR^{W18-1}—,
   wherein R^{W18-1} is H or R^{A}, and R^{W18-2} is H or R^{A1};
   W¹⁹, independently at each occurrence, is CR^{W19} or N, wherein R^{W19} is H or R^{A10};
   R^{W16} is hydrogen or R^{A10}, or R^{W16} and R^{W17-2} are taken together to form a double bond between the carbon atom bearing R^{W16} and the atom bearing R^{W17-2}, or R^{W16} and R^{W18-2} are taken together to form a double bond between the carbon atom bearing R^{W16} and the atom bearing R^{W18-2}.
C₆-C₁₄ aryl optionally substituted with 1, 2, 3, 4, 5, 6, 7, 8, or 9 R^{A10} substituents; and
5-14 membered heteroaryl optionally substituted with 1, 2, 3, 4, 5, 6, 7, 8, or 9 R^{A10} substituents;
R^{A10}, independently at each occurrence, is selected from the group consisting of halogen, NO₂, C₁-C₆ alkyl, C₂-C₆ alkenyl, C₂-C₆ alkynyl, C₁-C₆ haloalkyl, OH, O(C₁-C₆ alkyl), O(C₁-C₆ haloalkyl), SH, S(C₁-C₆ alkyl), S(C₁-C₆ haloalkyl), NH₂, NH(C₁-C₆ alkyl), NH(C₁-C₆ haloalkyl), N(C₁-C₆ alkyl)₂, N(C₁-C₆ haloalkyl)₂, NR^{a}R^{b}, CN, C(O)OH, C(O)O(C₁-C₆ alkyl), C(O)O(C₁-C₆ haloalkyl), C(O)NH₂, C(O)NH(C₁-C₆ alkyl), C(O)NH(C₁-C₆ haloalkyl), C(O)N(C₁-C₆ alkyl)₂, C(O)N(C₁-C₆ haloalkyl)₂, C(O)NR^{a}R^{b}, S(O)₂OH, S(O)₂O(C₁-C₆ alkyl), S(O)₂O(C₁-C₆ haloalkyl), S(O)₂NH₂, S(O)₂NH(C₁-C₆ alkyl), S(O)₂NH(C₁-C₆ haloalkyl), S(O)₂N(C₁-C₆ alkyl)₂, S(O)₂N(C₁-C₆ haloalkyl)₂, S(O)₂NR^{a}R^{b}, OC(O)H, OC(O)(C₁-C₆ alkyl), OC(O)(C₁-C₆ haloalkyl), N(H)C(O)H, N(H)C(O)(C₁-C₆ alkyl), N(H)C(O)(C₁-C₆ haloalkyl), N(C₁-C₆ alkyl)C(O)H, N(C₁-C₆ alkyl)C(O)(C₁-C₆ alkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ alkyl), OS(O)₂(C₁-C₆ haloalkyl), N(H)S(O)₂(C₁-C₆ alkyl), N(H)S(O)₂(C₁-C₆ haloalkyl), N(C₁-C₆ alkyl)S(O)₂(C₁-C₆ alkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl); wherein R^{a} and R^{b} are taken together with the nitrogen atom to which they are attached to form a 3-10 membered heterocycle;
   provided that when L⁹ is

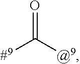

then A⁹ is (A⁹-1).

In some embodiments of the compounds of formula (E-1), or the salts thereof, R³⁶, R³⁷, R³⁸, R³⁹, R⁴⁰, R⁴¹, R⁴², and R⁴³ are each hydrogen. In some embodiments, R³⁹ and R⁴³ are taken together to form a C₁-C₆ alkylene moiety. In some embodiments, R³⁹ and R⁴³ are taken together to form a moiety selected from methylene, ethylene, and propylene. In some embodiments, R³⁹ and R⁴³ are taken together to form a methylene moiety. In some embodiments, R³⁹ and R⁴³ are taken together to form an ethylene moiety. In some embodiments, R³⁶, R³⁷, R³⁸, R⁴⁰, R⁴¹, and R⁴² are each hydrogen, and R³⁹ and R⁴³ are taken together to form a C₁-C₆ alkylene moiety. In some embodiments, R³⁶, R³⁷, R³⁸, R⁴⁰, R⁴¹, and R⁴² are each hydrogen, and R³⁹ and R⁴³ are taken together to form a moiety selected from methylene, ethylene, and propylene. In some embodiments, R³⁶, R³⁷, R³⁸, R⁴⁰, R⁴¹, and R⁴² are each hydrogen, and R³⁹ and R⁴³ are taken together to form a methylene moiety. In some embodiments, R³⁶, R³⁷, R³⁸, R⁴⁰, R⁴¹, and R⁴² are each hydrogen, and R³⁹ and R⁴³ are taken together to form an ethylene moiety.

In some embodiments of the compounds of formula (E-1), or the salts thereof, L⁹ is selected from the group consisting of a bond,

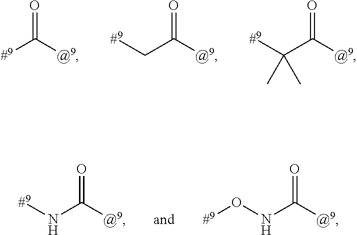

wherein #⁹ represents to attachment point to A⁹ and @⁹ represents the attachment point to the remainder of the molecule. In some embodiments, L⁹ is a bond. In some embodiments, L⁹ is

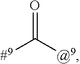

wherein #⁹ represents to attachment point to A⁹ and @⁹ represents the attachment point to the remainder of the molecule. In some embodiments, L⁹ is a bond,

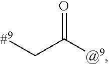

wherein #⁹ represents to attachment point to A⁹ and @⁹ represents the attachment point to the remainder of the molecule. In some embodiments, L⁹ is

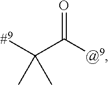

wherein #⁹ represents to attachment point to A⁹ and @⁹ represents the attachment point to the remainder of the molecule. In some embodiments, L⁹ is

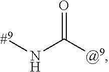

wherein #⁹ represents to attachment point to A⁹ and @⁹ represents the attachment point to the remainder of the molecule. In some embodiments, L⁹ is

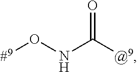

wherein #⁹ represents to attachment point to A⁹ and @⁹ represents the attachment point to the remainder of the molecule.

In some embodiments of the compounds of formula (E-1), or the salts thereof, L¹⁰ is selected from the group consisting of

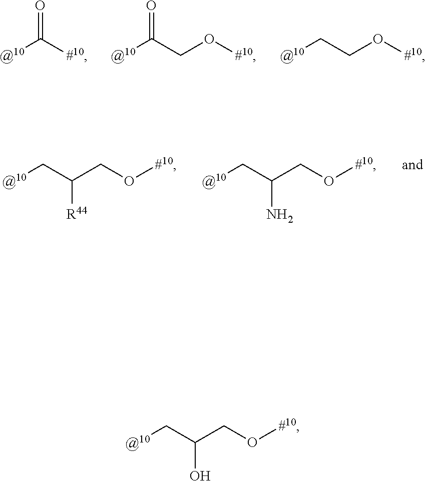

wherein #¹⁰ represents to attachment point to A¹⁰ and @¹⁰ represents the attachment point to the remainder of the molecule. In some embodiments L¹⁰ is

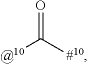

wherein #¹⁰ represents to attachment point to A¹⁰ and @¹⁰ represents the attachment point to the remainder of the molecule. In some embodiments L¹⁰ is

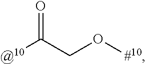

wherein #¹⁰ represents to attachment point to A¹⁰ and @¹⁰ represents the attachment point to the remainder of the molecule. In some embodiments L¹⁰ is

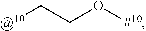

wherein #¹⁰ represents to attachment point to A¹⁰ and @¹⁰ represents the attachment point to the remainder of the molecule. In some embodiments L¹⁰ is

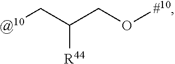

wherein #¹⁰ represents to attachment point to A¹⁰ and @¹⁰ represents the attachment point to the remainder of the molecule. In some embodiments L¹⁰ is

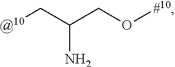

wherein #¹⁰ represents to attachment point to A¹⁰ and @¹⁰ represents the attachment point to the remainder of the molecule. In some embodiments L¹⁰ is

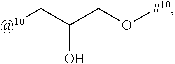

wherein #¹⁰ represents to attachment point to A¹⁰ and @¹⁰ represents the attachment point to the remainder of the molecule.

In some embodiments of the compounds of formula (E-1), or the salts thereof, R⁴⁴ is H, OH, or NH₂. In some embodiments, R⁴ is OH or NH₂. In some embodiments, R⁴ is H. In some embodiments, R⁴ is OH. In some embodiments, R⁴ is NH₂.

In some embodiments of the compounds of formula (E-1), or the salts thereof, A⁹ is a substituent of formula (A⁹-1)

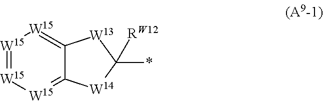

wherein * represents the attachment point to the remainder of the molecule;
W¹³ is selected from the group consisting of —C(R^{W13-1}R^{W13-2})—, —N(R^{W13-2})—, —C(R^{W13-1}R^{W13-2})N(R^{W13-2})—, —N(R^{W13-1})C(R^{W13-1}R^{W13-2})—, —C(R^{W13-1})═N—, —N═C(R^{W13-1})—, —O—, —C(R^{W13-1}R^{W13-1})O—, —OC(R^{W13-1}R^{W13-2})—, —S—, —C(R^{W13-1}R^{W13-1})S—, —SC(R^{W13-1}R^{W13-2})—, —C(R^{W13-1}R^{W13-1})C(R^{W13-1}R^{W13-2})—, and —CR^{W13-1}═CR^{W13-1}—,
wherein R^{W13-1} is H or R^{A9}, and R^{W13-2} is H or R^{A9}.
W¹⁴ is selected from the group consisting of —C(R^{W14-1}R^{W14-2})—, —N(R^{W14-2})—, —C(R^{W14-1}R^{W14-1})N(R^{W14-2})—, —N(R^{W14-1})C(R^{W14-1}R^{W14-2})—, —C(R^{W14-1})═N—, —N═C(R^{W14-1})—, —O—, —C(R^{W14-1}R^{W14-1})O—, —OC(R^{W14-1}R^{W14-2})—, —S—, —C(R^{W14-1}R^{W14-1})S—, —SC(R^{W14-1}R^{W14-2})—, —C(R^{W14-1}R^{W14-1})C(R^{W14-1}R^{W14-2})—, and —CR^{W14-1}═CR^{W14-1}—,
wherein R^{W14-1} is H or R^{A9}, and R^{W14-2} is H or R^{A9};
W¹⁵, independently at each occurrence, is CR^{W15} or N, wherein R^{W15} is H or R^{A9}; R^{W12} is hydrogen or R^{A9}, or R^{W12} and R^{W13-2} are taken together to form a double bond between the carbon atom bearing R^{W12} and the atom bearing R^{W13-2}, or R^{W12} and R^{W14-2} are taken together to form a double bond between the carbon atom bearing R^{W12} and the atom bearing R^{W14-2}.

In some embodiments, (A⁹-1) is selected from the group consisting of

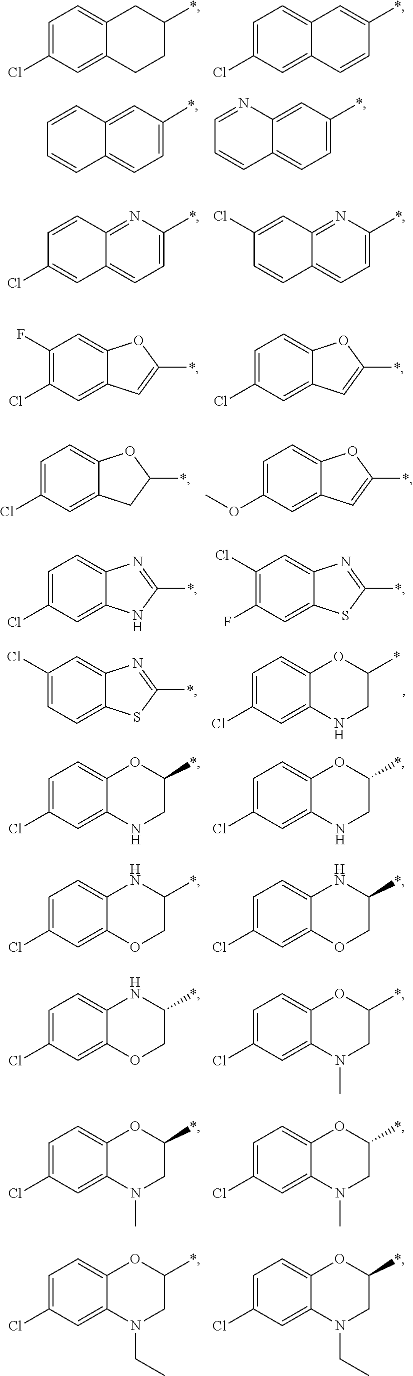

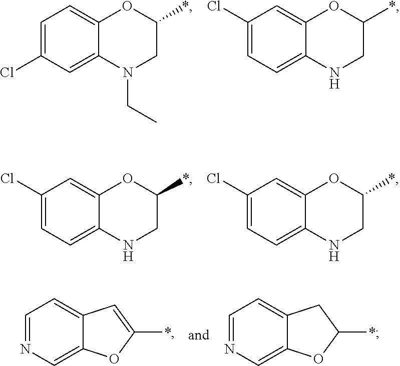

wherein * represents the attachment point to the remainder of the molecule. In some embodiments, (A⁹-1) is selected from the group consisting of

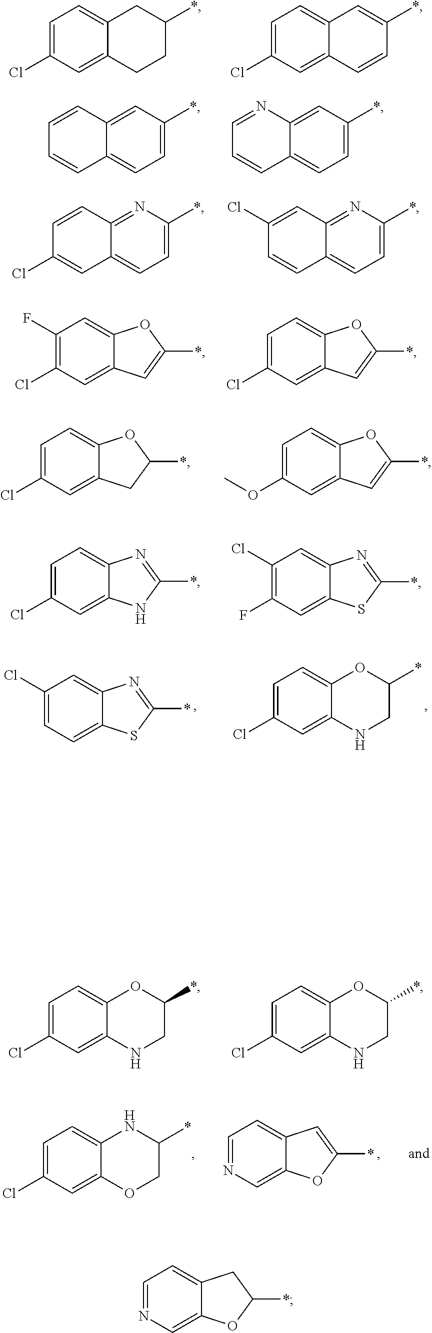

wherein * represents the attachment point to the remainder of the molecule. In some embodiments, (A⁹-1) is

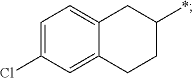

wherein * represents the attachment point to the remainder of the molecule. In some embodiments, (A⁹-1) is

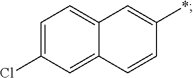

wherein * represents the attachment point to the remainder of the molecule. In some embodiments, (A⁹-1) is

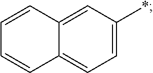

wherein * represents the attachment point to the remainder of the molecule. In some embodiments, (A⁹-1) is

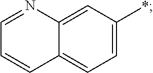

wherein * represents the attachment point to the remainder of the molecule. In some embodiments, (A⁹-1) is

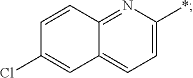

wherein * represents the attachment point to the remainder of the molecule. In some embodiments, (A⁹-1) is

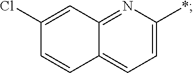

wherein * represents the attachment point to the remainder of the molecule. In some embodiments, (A⁹-1) is

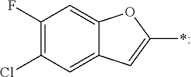

wherein * represents the attachment point to the remainder of the molecule. In some embodiments, (A⁹-1) is

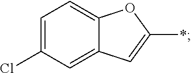

wherein * represents the attachment point to the remainder of the molecule. In some embodiments, (A⁹-1) is

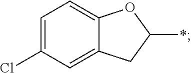

wherein * represents the attachment point to the remainder of the molecule. In some embodiments, (A⁹-1) is

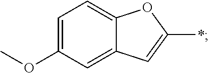

wherein * represents the attachment point to the remainder of the molecule. In some embodiments, (A⁹-1) is

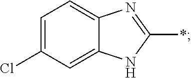

wherein * represents the attachment point to the remainder of the molecule. In some embodiments, (A⁹-1) is

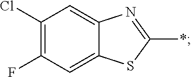

wherein * represents the attachment point to the remainder of the molecule. In some embodiments, (A⁹-1) is

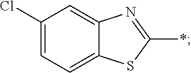

wherein * represents the attachment point to the remainder of the molecule. In some embodiments, (A⁹-1) is

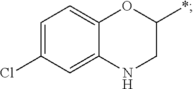

wherein * represents the attachment point to the remainder of the molecule. In some embodiments, (A⁹-1) is

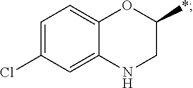

wherein * represents the attachment point to the remainder of the molecule. In some embodiments, (A⁹-1) is

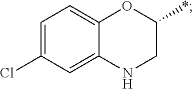

wherein * represents the attachment point to the remainder of the molecule. In some embodiments, (A⁹-1) is

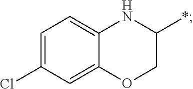

wherein * represents the attachment point to the remainder of the molecule. In some embodiments, (A⁹-1) is

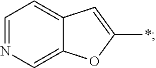

wherein * represents the attachment point to the remainder of the molecule. In some embodiments, (A⁹-1) is

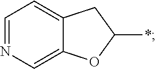

wherein * represents the attachment point to the remainder of the molecule.

In some embodiments of the compounds of formula (E-1), or the salts thereof, A⁹ is C₆-C₁₄ aryl optionally substituted with 1, 2, 3, 4, 5, 6, 7, 8, or 9 R^{A9} substituents. In some embodiments, A⁹ is selected from the group consisting of

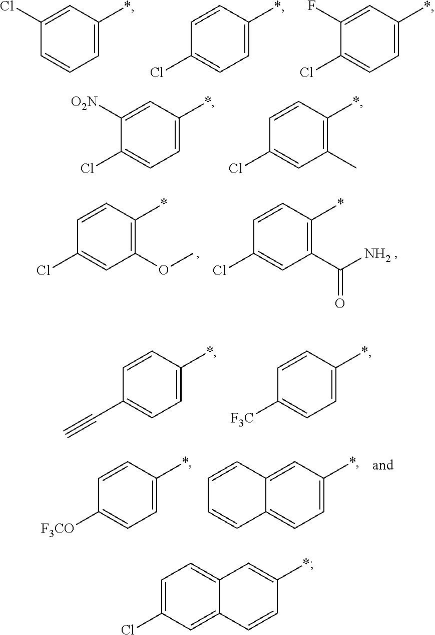

wherein * represents the attachment point to the remainder of the molecule. In some embodiments, A⁹ is selected from the group consisting of

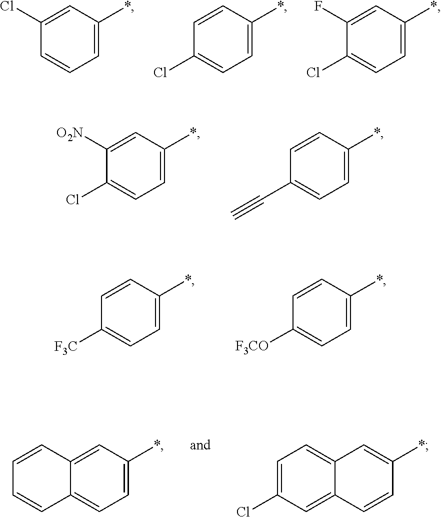

wherein * represents the attachment point to the remainder of the molecule. In some embodiments, A⁹ is

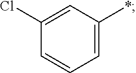

wherein * represents the attachment point to the remainder of the molecule. In some embodiments, A⁹ is

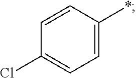

wherein * represents the attachment point to the remainder of the molecule. In some embodiments, A⁹ is

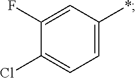

wherein * represents the attachment point to the remainder of the molecule. In some embodiments, A⁹ is

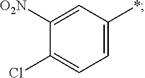

wherein * represents the attachment point to the remainder of the molecule. In some embodiments, A⁹ is

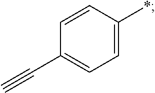

wherein * represents the attachment point to the remainder of the molecule. In some embodiments, A⁹ is

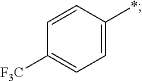

wherein * represents the attachment point to the remainder of the molecule. In some embodiments, A⁹ is

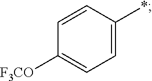

wherein * represents the attachment point to the remainder of the molecule. In some embodiments, A⁹ is

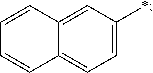

wherein * represents the attachment point to the remainder of the molecule. In some embodiments, A⁹ is

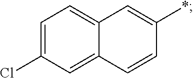

wherein * represents the attachment point to the remainder of the molecule.

In some embodiments of the compounds of formula (E-1), or the salts thereof, A⁹ is 5-14 membered heteroaryl optionally substituted with 1, 2, 3, 4, 5, 6, 7, 8, or 9 R^{A9} substituents. In some embodiments, A⁹ is selected from the group consisting of

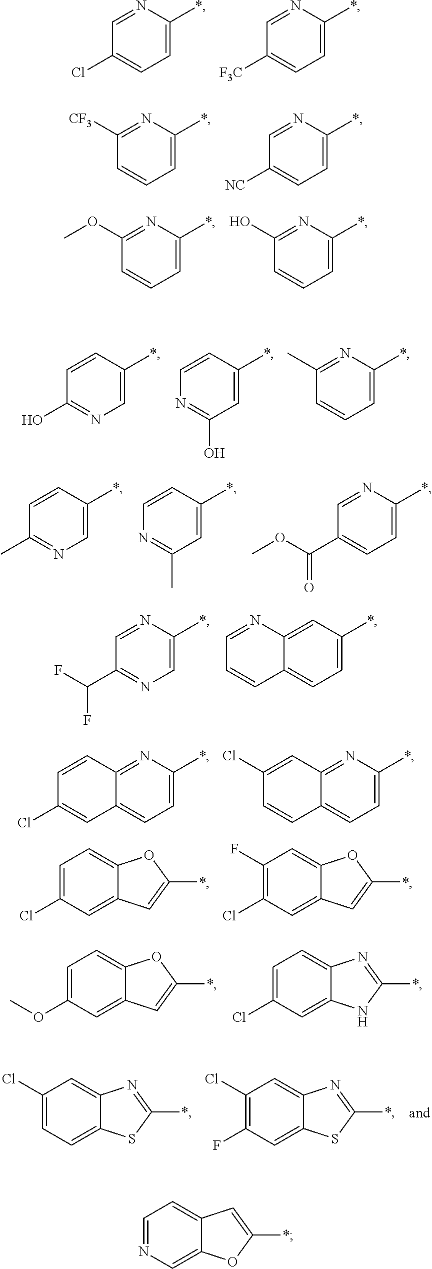

wherein * represents the attachment point to the remainder of the molecule. In some embodiments, A⁹ is selected from the group consisting of

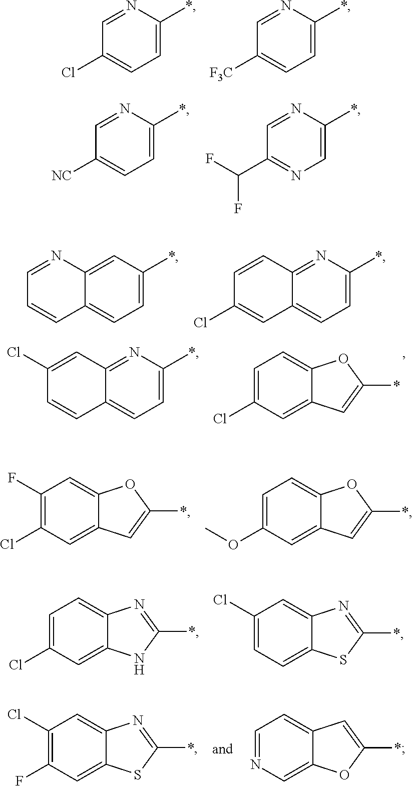

wherein * represents the attachment point to the remainder of the molecule. In some embodiments, A⁹ is

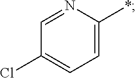

wherein * represents the attachment point to the remainder of the molecule. In some embodiments, A⁹ is

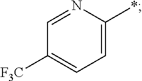

wherein * represents the attachment point to the remainder of the molecule. In some embodiments, A⁹ is

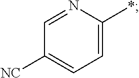

wherein * represents the attachment point to the remainder of the molecule. In some embodiments, A⁹ is

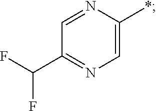

wherein * represents the attachment point to the remainder of the molecule. In some embodiments, A⁹ is

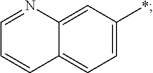

wherein * represents the attachment point to the remainder of the molecule. In some embodiments, A⁹ is

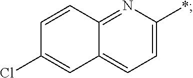

wherein * represents the attachment point to the remainder of the molecule. In some embodiments, A⁹ is

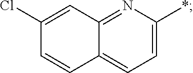

wherein * represents the attachment point to the remainder of the molecule. In some embodiments, A⁹ is

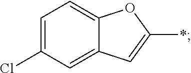

wherein * represents the attachment point to the remainder of the molecule. In some embodiments, A⁹ is

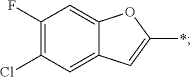

wherein * represents the attachment point to the remainder of the molecule. In some embodiments, A⁹ is

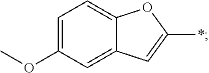

wherein * represents the attachment point to the remainder of the molecule. In some embodiments, A⁹ is

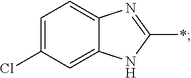

wherein * represents the attachment point to the remainder of the molecule. In some embodiments, A⁹ is

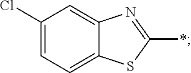

wherein * represents the attachment point to the remainder of the molecule. In some embodiments, A⁹ is

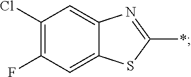

wherein * represents the attachment point to the remainder of the molecule. In some embodiments, A⁹ is

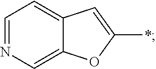

wherein * represents the attachment point to the remainder of the molecule.

In some embodiments of the compounds of formula (E-1), or the salts thereof, A¹⁰ is a substituent of formula (A¹⁰-1)

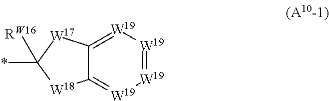

wherein * represents the attachment point to the remainder of the molecule;
W¹⁷ is selected from the group consisting of —C(R^{W17-1}R^{W17-2})—, —N(R^{W17-2})—, —C(R^{W17-1}R^{W17-2})N(R^{W17-2})—, —N(R^{W17-1})C(R^{W17-1}R^{W17-2})—, —C(R^{W17-1})═N—, —N═C(R^{W17-1})—, —O—, —C(R^{W17-1}R^{W17-1})O—, —OC(R^{W17-1}R^{W17-2})—, —S—, —C(R^{W17-1}R^{W17-1})S—, —SC(R^{W17-1}R^{W17-2})—, —C(R^{W17-1}R^{W17-1})C(R^{W17-1}R^{W17-2})—, and —CR^{W17-1}═CR^{W17-1}—,

wherein R^{W17-1} is H or R^{A10}, and R^{W17-2} is H or R^{A10};

W¹⁸ is selected from the group consisting of —C(R^{W18-1}R^{W18-2})—, —N(R^{W18-2})—, —C(R^{W18-1}R^{W18-1})N(R^{W18-2})—, —N(R^{W18-1})C(R^{W18-1}R^{W18-2})—, —C(R^{W18-1})═N—, —N═C(R^{W18-1})—, —O—, —C(R^{W18-1}R^{W18-1})O—, —OC(R^{W18-1}R^{W18-2})—, —S—, —C(R^{W18-1}R^{W18-1})S—, —SC(R^{W18-1}R^{W18-2})—, —C(R^{W18-1}R^{W18-1})C(R^{W18-1}R^{W18-2})—, and —CR^{W18-1}═CR^{W18-1}—,

wherein R^{W18-1} is H or R^{A10}, and R^{W18-2} is H or R^{A10};

W¹⁹, independently at each occurrence, is CR^{W19} or N, wherein R^{W19} is H or R^{A1};
R^{W16} is hydrogen or R^{A10}, or R^{W16} and R^{W7-2} are taken together to form a double bond between the carbon atom bearing R^{W16} and the atom bearing R^{W7-2}, or R^{W16} and R^{W18-2} are taken together to form a double bond between the carbon atom bearing R^{W16} and the atom bearing R^{W18-2};

In some embodiments, (A¹⁰-1) is selected from the group consisting of

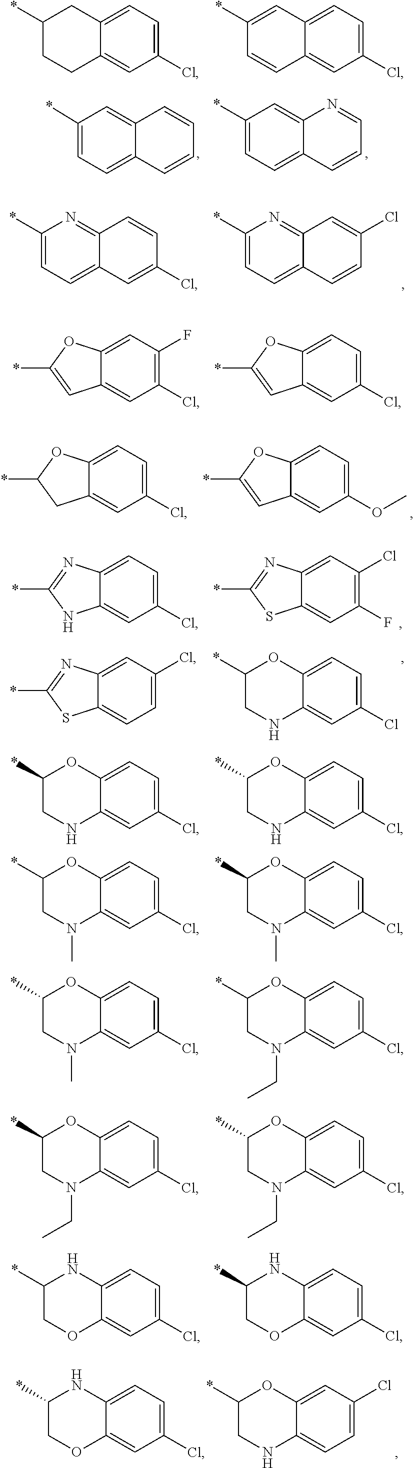

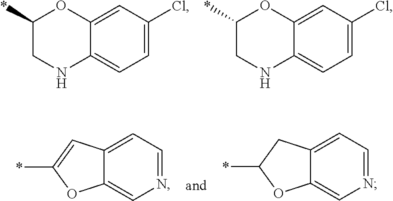

wherein * represents the attachment point to the remainder of the molecule. In some embodiments, (A¹⁰-1) is selected from the group consisting of

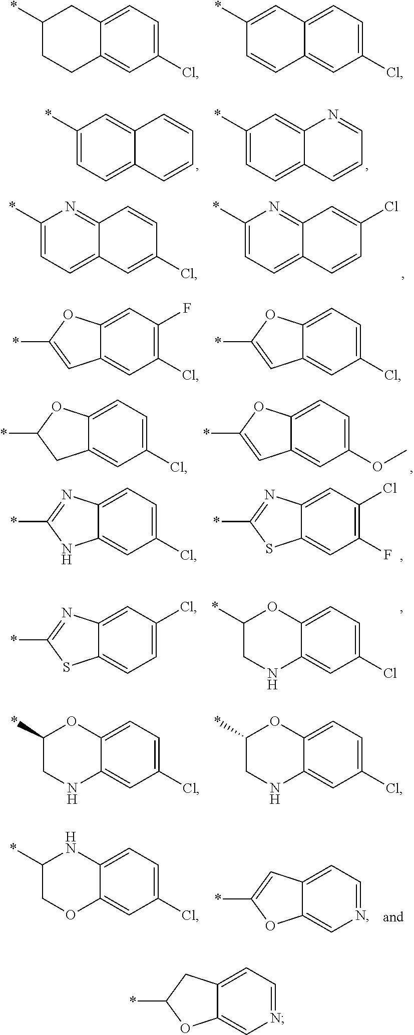

wherein * represents the attachment point to the remainder of the molecule. In some embodiments, (A¹⁰-1) is

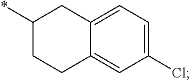 [SMILES: *C1CCc2cc(Cl)ccc2C1]

wherein * represents the attachment point to the remainder of the molecule. In some embodiments, (A¹⁰-1) is

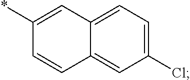 [SMILES: *c1ccc2cc(Cl)ccc2c1]

wherein * represents the attachment point to the remainder of the molecule. In some embodiments, (A¹⁰-1) is

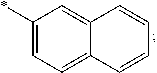 [SMILES: *c1ccc2ccccc2c1]

wherein * represents the attachment point to the remainder of the molecule. In some embodiments, (A¹⁰-1) is

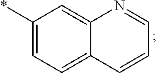 [SMILES: *c1ccc2cccnc2c1]

wherein * represents the attachment point to the remainder of the molecule. In some embodiments, (A¹⁰-1) is

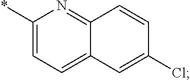 [SMILES: *c1ccc2cc(Cl)ccc2n1]

wherein * represents the attachment point to the remainder of the molecule. In some embodiments, (A¹⁰-1) is

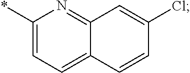 [SMILES: *c1ccc2ccc(Cl)cc2n1]

wherein * represents the attachment point to the remainder of the molecule. In some embodiments, (A¹⁰-1) is

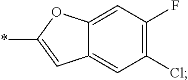 [SMILES: *c1cc2cc(Cl)c(F)cc2o1]

wherein * represents the attachment point to the remainder of the molecule. In some embodiments, (A¹⁰-1) is

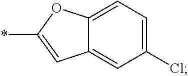 [SMILES: *c1cc2cc(Cl)ccc2o1]

wherein * represents the attachment point to the remainder of the molecule. In some embodiments, (A¹⁰-1) is

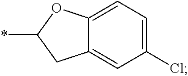 [SMILES: *C1Cc2cc(Cl)ccc2O1]

wherein * represents the attachment point to the remainder of the molecule. In some embodiments, (A¹⁰-1) is

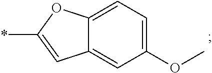 [SMILES: *c1cc2cc(OC)ccc2o1]

wherein * represents the attachment point to the remainder of the molecule. In some embodiments, (A¹⁰-1) is

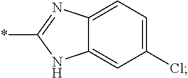 [SMILES: *c1nc2ccc(Cl)cc2[nH]1]

wherein * represents the attachment point to the remainder of the molecule. In some embodiments, (A¹⁰-1) is

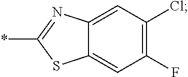 [SMILES: *c1nc2cc(Cl)c(F)cc2s1]

wherein * represents the attachment point to the remainder of the molecule. In some embodiments, (A¹⁰-1) is

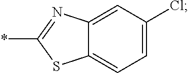 [SMILES: *c1nc2cc(Cl)ccc2s1]

wherein * represents the attachment point to the remainder of the molecule. In some embodiments, (A¹⁰-1) is

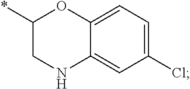 [SMILES: *C1CNc2cc(Cl)ccc2O1]

wherein * represents the attachment point to the remainder of the molecule. In some embodiments, (A¹⁰-1) is

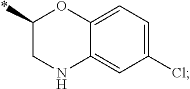 [SMILES: *[C@@H]1CNc2cc(Cl)ccc2O1]

wherein * represents the attachment point to the remainder of the molecule. In some embodiments, (A¹⁰-1) is

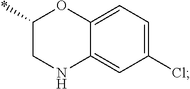 [SMILES: *[C@H]1CNc2cc(Cl)ccc2O1]

wherein * represents the attachment point to the remainder of the molecule. In some embodiments, (A¹⁰-1) is

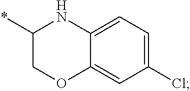 [SMILES: *C1COc2cc(Cl)ccc2N1]

wherein * represents the attachment point to the remainder of the molecule. In some embodiments, (A¹⁰-1) is

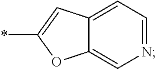 [SMILES: *c1cc2ccncc2o1]

wherein * represents the attachment point to the remainder of the molecule. In some embodiments, (A¹⁰-1) is

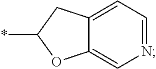 [SMILES: *C1Cc2ccncc2O1]

wherein * represents the attachment point to the remainder of the molecule.

In some embodiments of the compounds of formula (E-1), or the salts thereof, A¹⁰ is C₆-C₁₄ aryl optionally substituted with 1, 2, 3, 4, 5, 6, 7, 8, or 9 R^{A1} substituents. In some embodiments, A¹⁰ is selected from the group consisting of

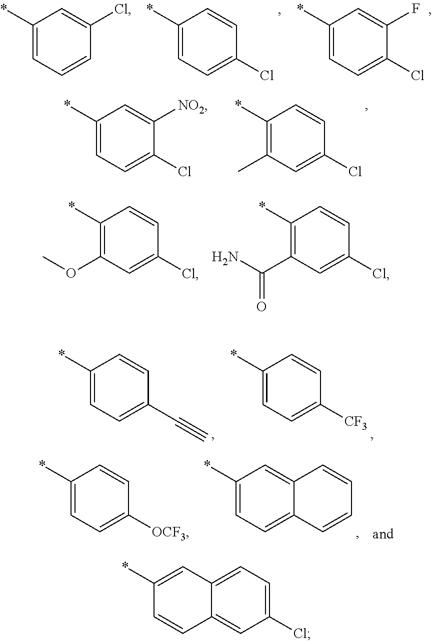

wherein * represents the attachment point to the remainder of the molecule. In some embodiments, A¹⁰ is selected from the group consisting of

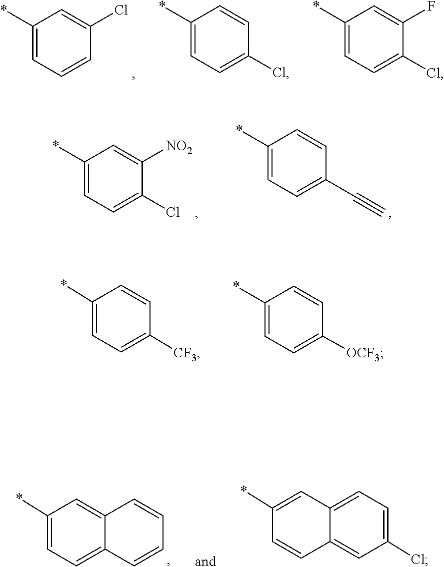

wherein * represents the attachment point to the remainder of the molecule. In some embodiments, A¹⁰ is

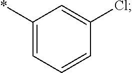 [SMILES: *c1cccc(Cl)c1]

wherein * represents the attachment point to the remainder of the molecule. In some embodiments, A¹⁰ is

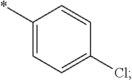 [SMILES: *c1ccc(Cl)cc1]

wherein * represents the attachment point to the remainder of the molecule. In some embodiments, A¹⁰ is

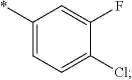 [SMILES: *c1ccc(Cl)c(F)c1]

wherein * represents the attachment point to the remainder of the molecule. In some embodiments, A¹⁰ is

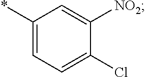 [SMILES: *c1ccc(Cl)c([N+](=O)[O-])c1]

wherein * represents the attachment point to the remainder of the molecule. In some embodiments, A¹⁰ is

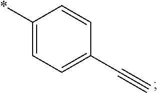 [SMILES: *c1ccc(C#C)cc1]

wherein * represents the attachment point to the remainder of the molecule. In some embodiments, A¹⁰ is

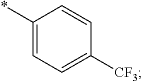 [SMILES: *c1ccc(C(F)(F)F)cc1]

wherein * represents the attachment point to the remainder of the molecule. In some embodiments, A¹⁰ is

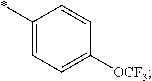 [SMILES: *c1ccc(OC(F)(F)F)cc1]

wherein * represents the attachment point to the remainder of the molecule. In some embodiments, A¹⁰ is

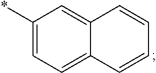 [SMILES: *c1ccc2ccccc2c1]

wherein * represents the attachment point to the remainder of the molecule. In some embodiments, A¹⁰ is

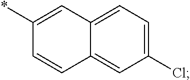 [SMILES: *c1ccc2cc(Cl)ccc2c1]

wherein * represents the attachment point to the remainder of the molecule.

In some embodiments of the compounds of formula (E-1), or the salts thereof, A¹⁰ is 5-14 membered heteroaryl optionally substituted with 1, 2, 3, 4, 5, 6, 7, 8, or 9 R^{A10} substituents. In some embodiments, A¹⁰ is selected from the group consisting of

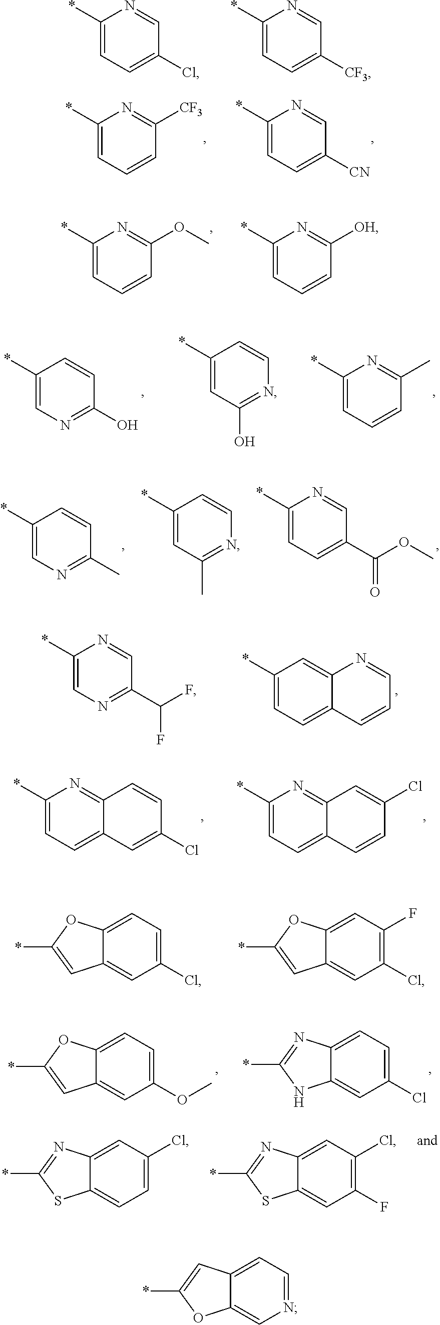

wherein * represents the attachment point to the remainder of the molecule. In some embodiments, A¹⁰ is selected from the group consisting of

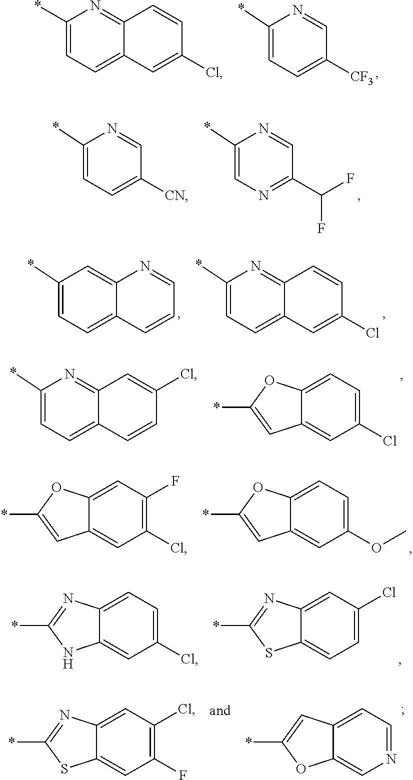

wherein * represents the attachment point to the remainder of the molecule. In some embodiments, A¹⁰ is

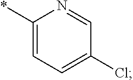 [SMILES: *c1ccc(Cl)cn1]

wherein * represents the attachment point to the remainder of the molecule. In some embodiments, A¹⁰ is

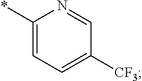 [SMILES: *c1ccc(C(F)(F)F)cn1]

wherein * represents the attachment point to the remainder of the molecule. In some embodiments, A¹⁰ is

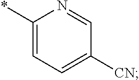 [SMILES: *c1ccc(C#N)cn1]

wherein * represents the attachment point to the remainder of the molecule. In some embodiments, A¹⁰ is

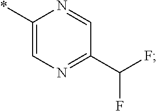 [SMILES: *c1cnc(C(F)F)cn1]

wherein * represents the attachment point to the remainder of the molecule. In some embodiments, A¹⁰ is

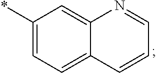 [SMILES: *c1ccc2cccnc2c1]

wherein * represents the attachment point to the remainder of the molecule. In some embodiments, A¹⁰ is

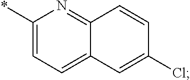 [SMILES: *c1ccc2cc(Cl)ccc2n1]

wherein * represents the attachment point to the remainder of the molecule. In some embodiments, A¹⁰ is

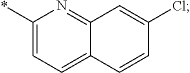 [SMILES: *c1ccc2ccc(Cl)cc2n1]

wherein * represents the attachment point to the remainder of the molecule. In some embodiments, A¹⁰ is

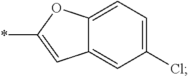 [SMILES: *c1cc2cc(Cl)ccc2o1]

wherein * represents the attachment point to the remainder of the molecule. In some embodiments, A¹⁰ is

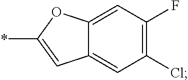 [SMILES: *c1cc2cc(Cl)c(F)cc2o1]

wherein * represents the attachment point to the remainder of the molecule. In some embodiments, A¹⁰ is

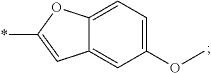 [SMILES: *c1cc2cc(OC)ccc2o1]

wherein * represents the attachment point to the remainder of the molecule. In some embodiments, A¹⁰ is

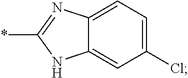 [SMILES: *c1nc2ccc(Cl)cc2[nH]1]

wherein * represents the attachment point to the remainder of the molecule. In some embodiments, A¹⁰ is

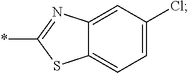 [SMILES: *c1nc2cc(Cl)ccc2s1]

wherein * represents the attachment point to the remainder of the molecule. In some embodiments, A¹⁰ is

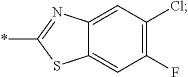 [SMILES: *c1nc2cc(Cl)c(F)cc2s1]

wherein * represents the attachment point to the remainder of the molecule. In some embodiments, A¹⁰ is

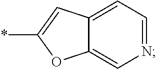 [SMILES: *c1cc2ccncc2o1]

wherein * represents the attachment point to the remainder of the molecule.

In an eight aspect, provided is a compound of formula (F-1)

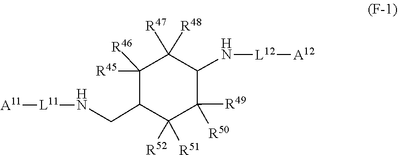

or a pharmaceutically acceptable salt thereof,
wherein:
R⁴⁵, R⁴⁶, R⁴⁷, R⁴⁸, R⁴⁹, R⁵⁰, R⁵¹, and R⁵², independently from each other, are selected from the group consisting of hydrogen, C₁-C₆ alkyl, C₁-C₆ haloalkyl, —C(O)OH, —C(O)O(C₁-C₆ alkyl), —C(O)O(C₁-C₆ haloalkyl), and halogen;
or, one of R⁴⁵, R⁴⁶, R⁴⁷, R⁴⁸, R⁴⁹, R⁵⁰, R⁵¹, and R⁵², and another one of R⁴⁵, R⁴⁶, R⁴⁷, R⁴⁸, R⁴⁹, R⁵⁰, R⁵¹, and R⁵², are taken together to form a C₁-C₆ alkylene moiety;
or, two geminal substituents selected from the group consisting of R⁴⁵, R⁴⁶, R⁴⁷, R⁴⁸, R⁴⁹, R⁵⁰, R⁵¹, and R⁵² are taken together to form an oxo group;
L¹¹ is selected from the group consisting of a bond,

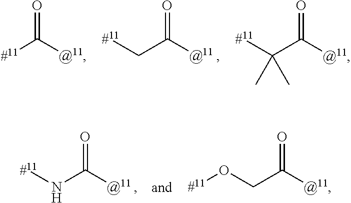

wherein #¹¹ represents to attachment point to A¹¹ and @¹¹ represents the attachment point to the remainder of the molecule;
L¹² is selected from the group consisting of

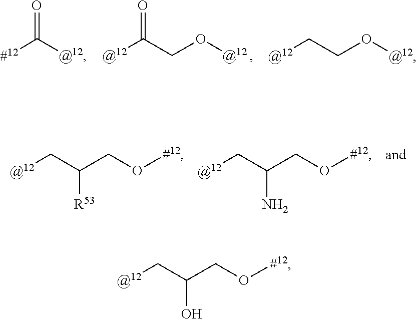

wherein #¹² represents to attachment point to A¹² and @¹² represents the attachment point to the remainder of the molecule;
R⁵³ is H, OH, or NH₂;
A¹¹ is selected from the group consisting of:
   a substituent of formula (A¹¹-1)

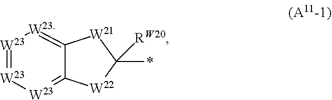

wherein * represents the attachment point to the remainder of the molecule;
   W²¹ is selected from the group consisting of —C(R^{W21-1}R^{W21-2})—, —N(R^{W21-2})—, C(R^{W21-1}R^{W21-2})N(R^{W21-2})—, N(R^{W21-1})C(R^{W21-1}R^{W21-2})—, —C(R^{W21-1})═N—, —N═C(R^{W21-1})—, —O—, —C(R^{W21-1}R^{W21-1})O—, —OC(R^{W21-1}R^{W21-2})—, —S—, —C(R^{W21-1}R^{W21-1})S—, —SC(R^{W21-1}R^{W21-2})—, —C(R^{W21-1}R^{W21-1})C(R^{W21-1}R^{W21-2})—, and —CR^{W21-1}═CR^{W21-1}—,
      wherein R^{W21-1} is H or R^{A1}, and R^{W21-2} is H or R^{A11};
   W²² is selected from the group consisting of —C(R^{W22-1}R^{W22-2}), —N(R^{W22-2})—, —C(R^{W22-1}R^{W22-1})N(R^{W22-2})—, —N(R^{W22-1})C(R^{W22-1}R^{W22-2})—, —C(R^{W22-1})═N—, —N═C(R^{W22-1})—, —O—, —C(R^{W22-1}R^{W22-1})O—, —OC(R^{W22-1}R^{W22-2})—, —S—, —C(R^{W22-1}R^{W22-1})S—, —SC(R^{W22-1}R^{W22-2})—, —C(R^{W22-1}R^{W22-1})C(R^{W22-1}R^{W22-2})—, and —CR^{W22-1}═CR^{W22-1}—,
      wherein R^{W22-1} is H or R^{A1}, and R^{W22-2} is H or R^{A11};
   W²³, independently at each occurrence, is CR^{W23} or N, wherein R^{W23} is H or R^{A11}.
   R^{W20} is hydrogen or R^{A11}, or R^{W20} and R^{W21-2} are taken together to form a double bond between the carbon atom bearing R^{W20} and the atom bearing R^{W21-2}, or R^{W20} and R^{W22-2} are taken together to form a double bond between the carbon atom bearing R^{W20} and the atom bearing R^{W21-2}.
C₆-C₁₄ aryl optionally substituted with 1, 2, 3, 4, 5, 6, 7, 8, or 9 R^{A11} substituents; and
5-14 membered heteroaryl optionally substituted with 1, 2, 3, 4, 5, 6, 7, 8, or 9 R^{A11} substituents;
R^{A11}, independently at each occurrence, is selected from the group consisting of halogen, NO₂, C₁-C₆ alkyl, C₂-C₆ alkenyl, C₂-C₆ alkynyl, C₁-C₆ haloalkyl, OH, O(C₁-C₆ alkyl), O(C₁-C₆ haloalkyl), SH, S(C₁-C₆ alkyl), S(C₁-C₆ haloalkyl), NH₂, NH(C₁-C₆ alkyl), NH(C₁-C₆ haloalkyl), N(C₁-C₆ alkyl)₂, N(C₁-C₆ haloalkyl)₂, NR^{a}R^{b}, CN, C(O)OH, C(O)O(C₁-C₆ alkyl), C(O)O(C₁-C₆ haloalkyl), C(O)NH₂, C(O)NH(C₁-C₆ alkyl), C(O)NH(C₁-C₆ haloalkyl), C(O)N(C₁-C₆ alkyl)₂, C(O)N(C₁-C₆ haloalkyl)₂, C(O)NR^{a}R^{b}, S(O)₂OH, S(O)₂O(C₁-C₆ alkyl), S(O)₂O(C₁-C₆ haloalkyl), S(O)₂NH₂, S(O)₂NH(C₁-C₆ alkyl), S(O)₂NH(C₁-C₆ haloalkyl), S(O)₂N(C₁-C₆ alkyl)₂, S(O)₂N(C₁-C₆ haloalkyl)₂, S(O)₂NR^{a}R^{b}, OC(O)H, OC(O)(C₁-C₆ alkyl), OC(O)(C₁-C₆ haloalkyl), N(H)C(O)H, N(H)C(O)(C₁-C₆ alkyl), N(H)C(O)(C₁-C₆ haloalkyl), N(C₁-C₆ alkyl)C(O)H, N(C₁-C₆ alkyl)C(O)(C₁-C₆ alkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ alkyl), OS(O)₂(C₁-C₆ haloalkyl), N(H)S(O)₂(C₁-C₆ alkyl), N(H)S(O)₂(C₁-C₆ haloalkyl), N(C₁-C₆ alkyl)S(O)₂(C₁-C₆ alkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl); wherein R^{a} and R^{b} are taken together with the nitrogen atom to which they are attached to form a 3-10 membered heterocycle;
   and
A¹² is selected from the group consisting of:
   a substituent of formula (A¹²-1)

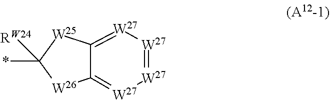

wherein * represents the attachment point to the remainder of the molecule;
   W²⁵ is selected from the group consisting of —C(R^{W25-1}R^{W25-2})—, —N(R^{W25-2})—, —C(R^{W25-1}R^{W25-2})N(R^{W25-2})—, —N(R^{W25-1})C(R^{W25-1}R^{W25-2})—, —C(R^{W25-1})═N—, —N═C(R^{W25-1})—, —O—, —C(R^{W25-1}R^{W25-1})O—, —OC(R^{W25-1}R^{W25-2})—, —S—, —C(R^{W25-1}R^{W25-1})S—, —SC(R^{W25-1}R^{W25-2})—, —C(R^{W25-1}R^{W25-1})C(R^{W25-1}R^{W25-2})—, and —CR^{W25-1}═CR^{W25-1}—,
   wherein R^{W25-1} is H or R^{A12}, and R^{W25-2} is H or R^{A12}.
   W²⁶ is selected from the group consisting of —C(R^{W26-1}R^{W26-2})—, —N(R^{W26-2})—, —C(R^{W26-1}R^{W26-1})N(R^{W26-2})—, —N(R^{W26-1})C(R^{W26-1}R^{W26-2})—, —C(R^{W26-1})═N—, —N═C(R^{W26-1})—, —O—, —C(R^{W26-1}R^{W26-1})O—, —OC(R^{W26-1}R^{W26-2})—, —S—, —C(R^{W26-1}R^{W26-1})S—, —SC(R^{W26-1}R^{W26-2})—, —C(R^{W26-1}R^{W26-1})C(R^{W26-1}R^{W26-2})—, and —CR^{W26-1}═CR^{W26-1}—,
   wherein R^{W26-1} is H or R^{A12}, and R^{W26-2} is H or R^{A12}.
   W²⁷, independently at each occurrence, is CR^{W27} or N, wherein R^{W27} is H or R^{A12}.
   R^{W24} is hydrogen or R^{A12}, or R^{W24} and R^{W25-2} are taken together to form a double bond between the carbon atom bearing R^{W24} and the atom bearing R^{W25-2}, or R^{W24} and R^{W26-2} are taken together to form a double bond between the carbon atom bearing R^{W24} and the atom bearing R^{W26-2}.
C₆-C₁₄ aryl optionally substituted with 1, 2, 3, 4, 5, 6, 7, 8, or 9 R^{A12} substituents; and
5-14 membered heteroaryl optionally substituted with 1, 2, 3, 4, 5, 6, 7, 8, or 9 R^{A12} substituents;
R^{A12}, independently at each occurrence, is selected from the group consisting of halogen, NO₂, C₁-C₆ alkyl, C₂-C₆ alkenyl, C₂-C₆ alkynyl, C₁-C₆ haloalkyl, OH, O(C₁-C₆ alkyl), O(C₁-C₆ haloalkyl), SH, S(C₁-C₆ alkyl), S(C₁-C₆ haloalkyl), NH₂, NH(C₁-C₆ alkyl), NH(C₁-C₆ haloalkyl), N(C₁-C₆ alkyl)₂, N(C₁-C₆ haloalkyl)₂, NR^{a}R^{b}, CN, C(O)OH, C(O)O(C₁-C₆ alkyl), C(O)O(C₁-C₆ haloalkyl), C(O)NH₂, C(O)NH(C₁-C₆ alkyl), C(O)NH(C₁-C₆ haloalkyl), C(O)N(C₁-C₆ alkyl)₂, C(O)N(C₁-C₆ haloalkyl)₂, C(O)NR^{a}R^{b}, S(O)₂OH, S(O)₂O(C₁-C₆ alkyl), S(O)₂O(C₁-C₆ haloalkyl), S(O)₂NH₂, S(O)₂NH(C₁-C₆ alkyl), S(O)₂NH(C₁-C₆ haloalkyl), S(O)₂N(C₁-C₆ alkyl)₂, S(O)₂N(C₁-C₆ haloalkyl)₂, S(O)₂NR^{a}R^{b}, OC(O)H, OC(O)(C₁-C₆ alkyl), OC(O)(C₁-C₆ haloalkyl), N(H)C(O)H, N(H)C(O)(C₁-C₆ alkyl), N(H)C(O)(C₁-C₆ haloalkyl), N(C₁-C₆ alkyl)C(O)H, N(C₁-C₆ alkyl)C(O)(C₁-C₆ alkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ alkyl), OS(O)₂(C₁-C₆ haloalkyl), N(H)S(O)₂(C₁-C₆ alkyl), N(H)S(O)₂(C₁-C₆ haloalkyl), N(C₁-C₆ alkyl)S(O)₂(C₁-C₆ alkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl); wherein R^{a} and R^{b} are taken together with the nitrogen atom to which they are attached to form a 3-10 membered heterocycle;
   provided that
when L¹¹ is a bond, then A¹¹ is (A¹¹-1) optionally substituted by 1, 2, 3, 4, 5, 6, 7, 8, or 9 R^{A11} substituents;
when L¹¹ is

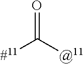

### and L¹² is

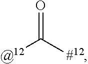

then A¹¹ is (A¹¹-1) substituted by 1, 2, 3, 4, 5, 6, 7, 8, or 9 R^{A11} substituents or A¹² is (A¹¹-1) substituted by 2, 3, 4, 5, 6, 7, 8, or 9 R^{A12} substituents;
and
when L¹¹ is

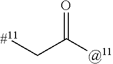

### and L¹² is

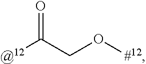

then A¹¹ is substituted by 1, 2, 3, 4, 5, 6, 7, 8, or 9 R^{A11} substituents.

In some embodiments of the compounds of formula (F-1), or the salts thereof, R⁴⁵, R⁴⁶, R⁴⁷, R⁴⁸, R⁴⁹, R⁵⁰, R⁵¹, and R⁵² are each hydrogen. In some embodiments, R⁴⁸ and R⁵² are taken together to form a C₁-C₆ alkylene moiety. In some embodiments, R⁴⁸ and R⁵² are taken together to form a moiety selected from methylene, ethylene, and propylene. In some embodiments, R⁴⁸ and R⁵² are taken together to form a methylene moiety. In some embodiments, R⁴⁸ and R⁵² are taken together to form an ethylene moiety. In some embodiments, R⁴⁵, R⁴⁶, R⁴⁷, R⁴⁹, R⁵⁰, and R⁵¹ are each hydrogen, and R⁴⁸ and R⁵² are taken together to form a C₁-C₆ alkylene moiety. In some embodiments, R⁴⁵, R⁴⁶, R⁴⁷, R⁴⁹, R⁵⁰, and R⁵¹ are each hydrogen, and R⁴⁸ and R⁵² are taken together to form a moiety selected from methylene, ethylene, and propylene. In some embodiments, R⁴⁵, R⁴⁶, R⁴⁷, R⁴⁹, R⁵⁰, and R⁵¹ are each hydrogen, and R⁴⁸ and R⁵² are taken together to form a methylene moiety. In some embodiments, R⁴⁵, R⁴⁶, R⁴⁷, R⁴⁹, R⁵⁰, and R⁵¹ are each hydrogen, and R⁴⁸ and R⁵² are taken together to form an ethylene moiety.

In some embodiments of the compounds of formula (F-1), or the salts thereof, L¹¹ is selected from the group consisting of a bond,

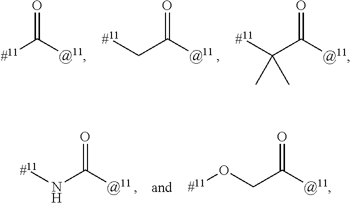

and #¹¹, wherein #¹¹ represents to attachment point to A¹¹ and @¹¹ represents the attachment point to the remainder of the molecule. In some embodiments, L¹¹ is a bond. In some embodiments, L¹¹ is

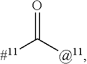

wherein #¹¹ represents to attachment point to A¹¹ and @¹¹ represents the attachment point to the remainder of the molecule. In some embodiments, L¹¹ is a bond,

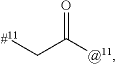

wherein #¹¹ represents to attachment point to A¹¹ and @¹¹ represents the attachment point to the remainder of the molecule. In some embodiments, L¹¹ is

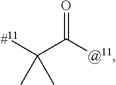

wherein #¹¹ represents to attachment point to A¹¹ and @¹¹ represents the attachment point to the remainder of the molecule. In some embodiments, L¹¹ is

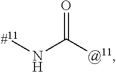

wherein #¹¹ represents to attachment point to A¹¹ and @¹¹ represents the attachment point to the remainder of the molecule. In some embodiments, L¹¹ is

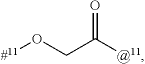

wherein #¹¹ represents to attachment point to A¹¹ and @¹¹ represents the attachment point to the remainder of the molecule.

In some embodiments of the compounds of formula (F-1), or the salts thereof, L¹² is selected from the group consisting of

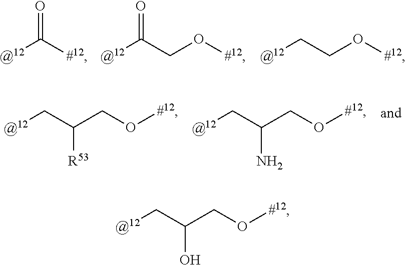

wherein #¹² represents to attachment point to A¹² and @¹² represents the attachment point to the remainder of the molecule. In some embodiments L¹² is

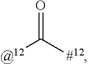

wherein #¹² represents to attachment point to A¹² and @¹² represents the attachment point to the remainder of the molecule. In some embodiments L¹² is

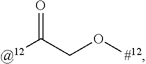

wherein #¹² represents to attachment point to A¹² and @¹² represents the attachment point to the remainder of the molecule. In some embodiments L¹² is

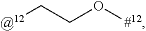

wherein #¹² represents to attachment point to A¹² and @¹² represents the attachment point to the remainder of the molecule. In some embodiments L¹² is

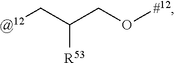

wherein #¹² represents to attachment point to A¹² and @¹² represents the attachment point to the remainder of the molecule. In some embodiments L¹² is

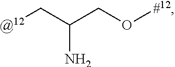

wherein #¹² represents to attachment point to A¹² and @¹² represents the attachment point to the remainder of the molecule. In some embodiments L¹² is

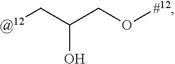

wherein #¹² represents to attachment point to A¹² and @¹² represents the attachment point to the remainder of the molecule.

In some embodiments of the compounds of formula (F-1), or the salts thereof, R⁵ is H, OH, or NH₂. In some embodiments, R⁵³ is OH or NH₂. In some embodiments, R⁵³ is H. In some embodiments, R⁵³ is OH. In some embodiments, R⁵³ is NH₂.

In some embodiments of the compounds of formula (F-1), or the salts thereof, A¹¹ is a substituent of formula (A¹¹-1)

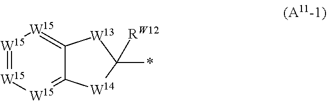

wherein * represents the attachment point to the remainder of the molecule;
W¹³ is selected from the group consisting of —C(R^{W13-1}R^{W3-2}), N(R^{W13-2})—, —C(R^{W13-1}R^{W13-2})N(R^{W13-2})—, —N(R^{W13-1})C(R^{W13-1}R^{W13-2})—, —C(R^{W13-1})═N—, —N═C(R^{W13-1})—, —O—, —C(R^{W13-1}R^{W13-1})O—, —OC(R^{W13-1}R^{W13-2})—, —S—, —C(R^{W13-1}R^{W13-1})S—, —SC(R^{W13-1}R^{W13-2})—, —C(R^{W13-1}R^{W13-1})C(R^{W13-1}R^{W13-2})—, and —CR^{W13-1}═CR^{W13}—,
wherein R^{W3}-1 is H or R^{A1}, and R^{W13-2} is H or R^{A11};
W¹⁴ is selected from the group consisting of —C(R^{W14-1}R^{W4-2}), N(R^{W14-2})—, —C(R^{W14-1}R^{W14-1})N(R^{W14-2})—, —N(R^{W14-1})C(R^{W14-1}R^{W14-2})—, —C(R^{W14-1})═N—, —N═C(R^{W14-1})—, —O—, —C(R^{W14-1}R^{W14-1})O—, —OC(R^{W14-1}R^{W14-2})—, —S—, —C(R^{W14-1}R^{W14-1})S—, —SC(R^{W14-1}R^{W14-2})—, —C(R^{W14-1}R^{W14-1})C(R^{W14-1}R^{W14-2})—, and —CR^{W14-1}═CR^{W14-1}—,
wherein R^{W14-1} is H or R^{A1}, and R^{W14-2} is H or R^{A11};
W¹⁵, independently at each occurrence, is CR^{W15} or N, wherein R^{W15} H or R^{A11};
R^{W12} is hydrogen or R^{A11}, or R^{W12} and R^{W13-2} are taken together to form a double bond between the carbon atom bearing R^{W12} and the atom bearing R^{W13-2}, or R^{W12} and R^{W14-2} are taken together to form a double bond between the carbon atom bearing R^{W12} and the atom bearing R^{W14-2}.

In some embodiments, (A¹¹-1) is selected from the group consisting of

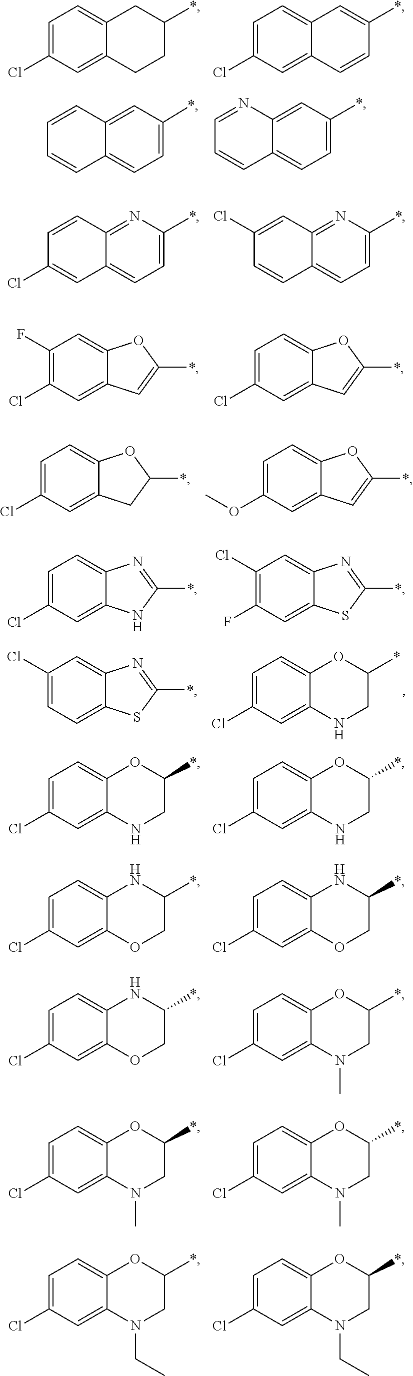

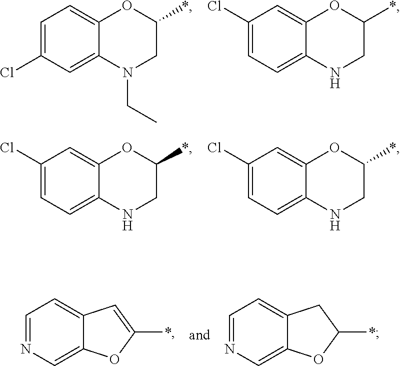

wherein * represents the attachment point to the remainder of the molecule. In some embodiments, (A¹-1) is selected from the group consisting of

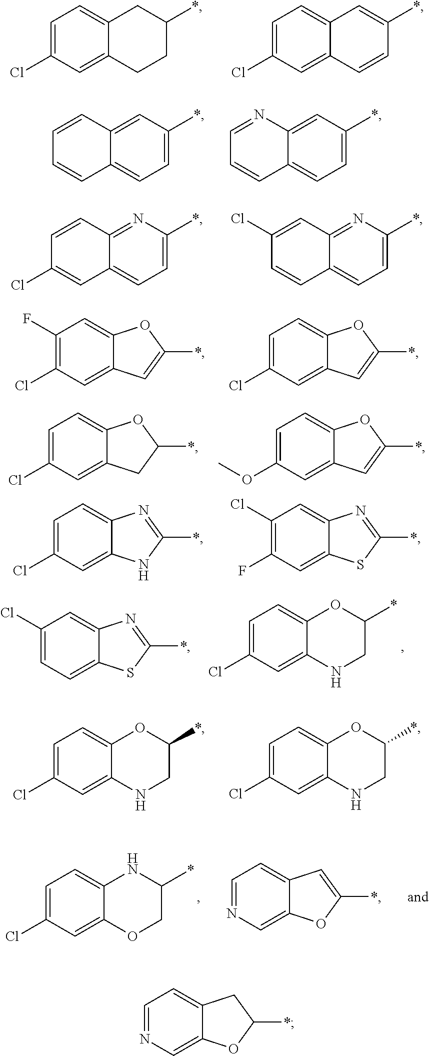

wherein * represents the attachment point to the remainder of the molecule. In some embodiments, (A¹¹-1) is

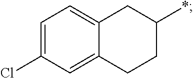

wherein * represents the attachment point to the remainder of the molecule. In some embodiments, (A¹¹-1) is

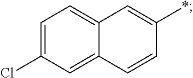

wherein * represents the attachment point to the remainder of the molecule. In some embodiments, (A¹¹-1) is

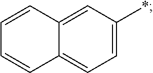

wherein * represents the attachment point to the remainder of the molecule. In some embodiments, (A¹¹-1) is

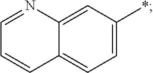

wherein * represents the attachment point to the remainder of the molecule. In some embodiments, (A¹¹-1) is

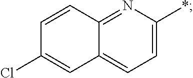

wherein * represents the attachment point to the remainder of the molecule. In some embodiments, (A¹¹-1) is

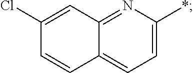

wherein * represents the attachment point to the remainder of the molecule. In some embodiments, (A¹¹-1) is

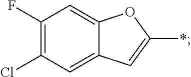

wherein * represents the attachment point to the remainder of the molecule. In some embodiments, (A¹¹-1) is

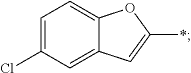

wherein * represents the attachment point to the remainder of the molecule. In some embodiments, (A¹¹-1) is

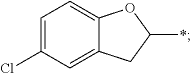

wherein * represents the attachment point to the remainder of the molecule. In some embodiments, (A¹¹-1) is

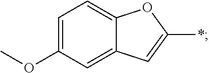

wherein * represents the attachment point to the remainder of the molecule. In some embodiments, (A¹¹-1) is

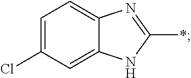

wherein * represents the attachment point to the remainder of the molecule. In some embodiments, (A¹¹-1) is

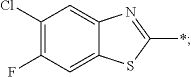

wherein * represents the attachment point to the remainder of the molecule. In some embodiments, (A¹¹-1) is

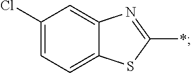

wherein * represents the attachment point to the remainder of the molecule. In some embodiments, (A¹¹-1) is

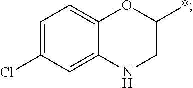

wherein * represents the attachment point to the remainder of the molecule. In some embodiments, (A¹¹-1) is

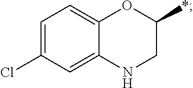

wherein * represents the attachment point to the remainder of the molecule. In some embodiments, (A¹¹-1) is

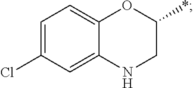

wherein * represents the attachment point to the remainder of the molecule. In some embodiments, (A¹¹-1) is

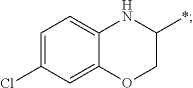

wherein * represents the attachment point to the remainder of the molecule. In some embodiments, (A¹¹-1) is

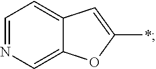

wherein * represents the attachment point to the remainder of the molecule. In some embodiments, (A¹¹-1) is

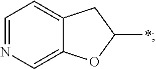

wherein * represents the attachment point to the remainder of the molecule.

In some embodiments of the compounds of formula (F-1), or the salts thereof, A¹¹ is C₆-C₁₄ aryl optionally substituted with 1, 2, 3, 4, 5, 6, 7, 8, or 9 R^{A11} substituents. In some embodiments, A¹¹ is selected from the group consisting of

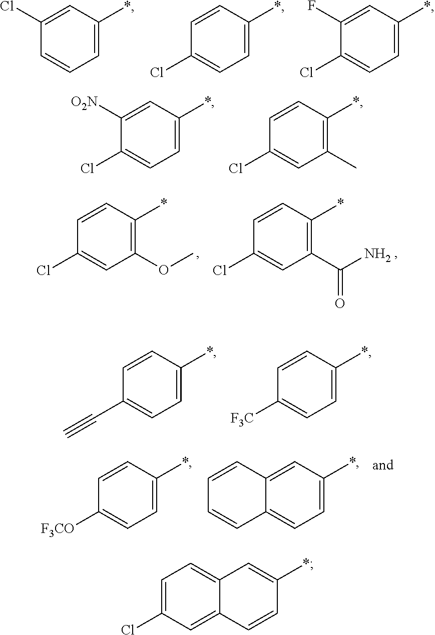

wherein * represents the attachment point to the remainder of the molecule. In some embodiments, A¹¹ is selected from the group consisting of

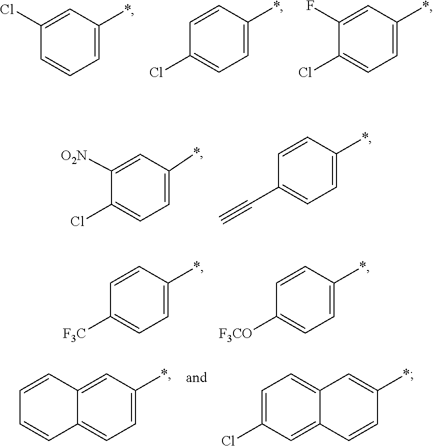

wherein * represents the attachment point to the remainder of the molecule. In some embodiments, A¹¹ is

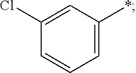

wherein * represents the attachment point to the remainder of the molecule. In some embodiments, A¹¹ is

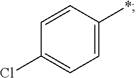

wherein * represents the attachment point to the remainder of the molecule. In some embodiments, A¹ is

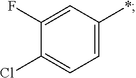

wherein * represents the attachment point to the remainder of the molecule. In some embodiments, A¹¹ is

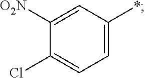

wherein * represents the attachment point to the remainder of the molecule. In some embodiments, A¹¹ is

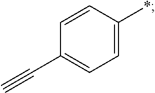

wherein * represents the attachment point to the remainder of the molecule. In some embodiments, A¹¹ is

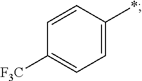

wherein * represents the attachment point to the remainder of the molecule. In some embodiments, A¹ is

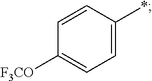

wherein * represents the attachment point to the remainder of the molecule. In some embodiments, A¹¹ is

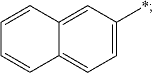

wherein * represents the attachment point to the remainder of the molecule. In some embodiments, A¹¹ is

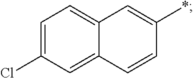

wherein * represents the attachment point to the remainder of the molecule.

In some embodiments of the compounds of formula (F-1), or the salts thereof, A¹¹ is 5-14 membered heteroaryl optionally substituted with 1, 2, 3, 4, 5, 6, 7, 8, or 9 R^{A11} substituents. In some embodiments, A¹¹ is selected from the group consisting of

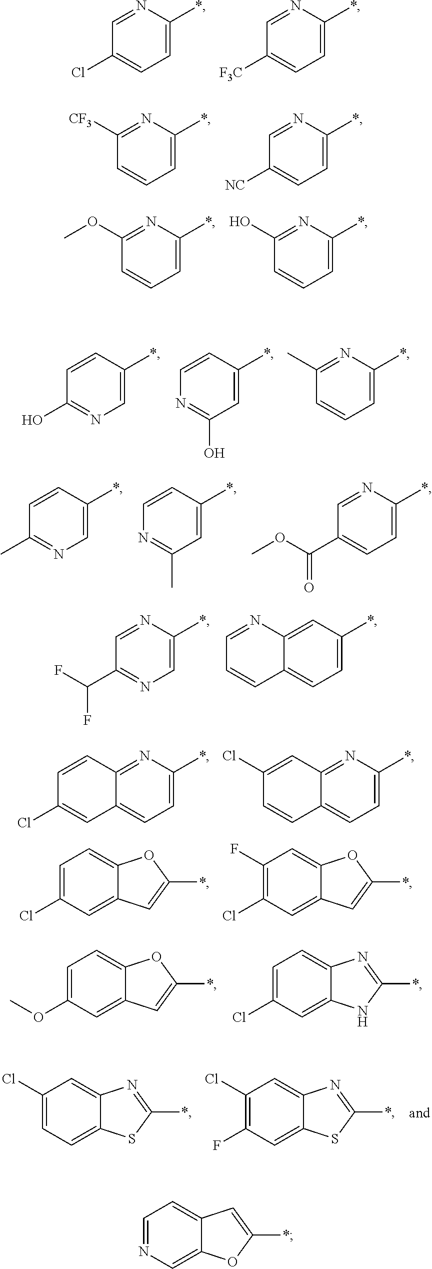

wherein * represents the attachment point to the remainder of the molecule. In some embodiments, A¹¹ is selected from the group consisting of

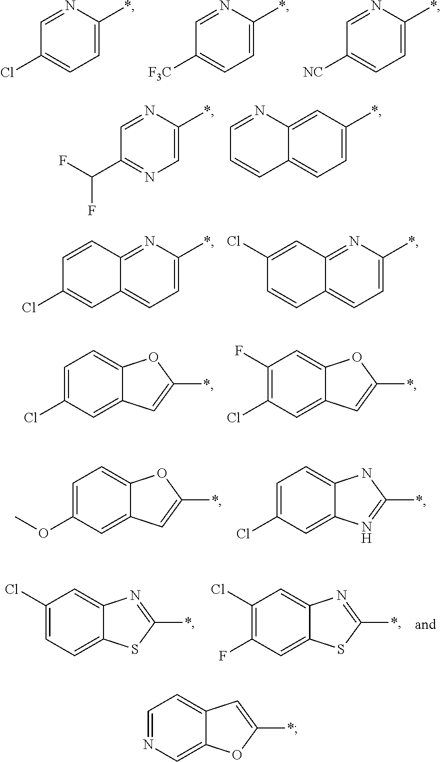

wherein * represents the attachment point to the remainder of the molecule. In some embodiments, A¹¹ is

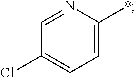

wherein * represents the attachment point to the remainder of the molecule. In some embodiments, A¹¹ is

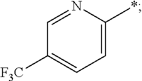

wherein * represents the attachment point to the remainder of the molecule. In some points, A¹¹ is

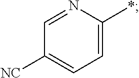

wherein * represents the attachment point to the remainder of the molecule. In some embodiments, A¹¹ is

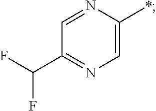

wherein * represents the attachment point to the remainder of the molecule. In some embodiments, A¹¹ is

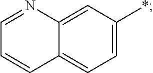

wherein * represents the attachment point to the remainder of the molecule. In some embodiments, A¹¹ is

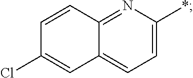

wherein * represents the attachment point to the remainder of the molecule. In some embodiments, A¹¹ is

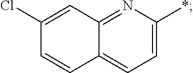

wherein * represents the attachment point to the remainder of the molecule. In some embodiments, A¹¹ is

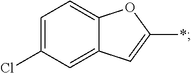

wherein * represents the attachment point to the remainder of the molecule. In some embodiments, A¹¹ is

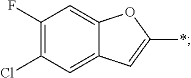

wherein * represents the attachment point to the remainder of the molecule. In some embodiments, A¹¹ is

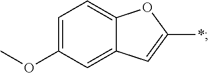

wherein * represents the attachment point to the remainder of the molecule. In some embodiments, A¹¹ is

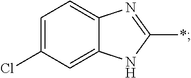

wherein * represents the attachment point to the remainder of the molecule. In some embodiments, A¹¹ is

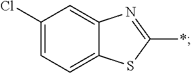

wherein * represents the attachment point to the remainder of the molecule. In some embodiments, A¹¹ is

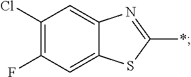

wherein * represents the attachment point to the remainder of the molecule. In some embodiments, A¹¹ is

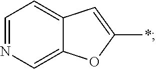

wherein * represents the attachment point to the remainder of the molecule.

In some embodiments of the compounds of formula (F-1), or the salts thereof, A¹² is a substituent of formula (A¹²-1)

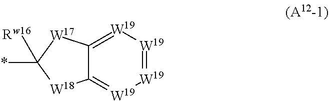

wherein * represents the attachment point to the remainder of the molecule;
W¹⁷ is selected from the group consisting of —C(R^{W17-1}R^{W17-2})—, —N(R^{W17-2})—, —C(R^{W17-1}R^{W17-2})N(R^{W17-2})—, —N(R^{W17-1})C(R^{W17-1}R^{W17-2})—, —C(R^{W17-1})═N—, —N═C(R^{W17-1})—, —O—, —C(R^{W17-1}R^{W17-1})O—, —OC(R^{W17-1}R^{W17-2})—, —S—, —C(R^{W17-1}R^{W17-1})S—, —SC(R^{W17-1}R^{W17-2})—, —C(R^{W17-1}R^{W17-1})C(R^{W17-1}R^{W17-2})—, and —CR^{W17-1}═CR^{W17}—,

wherein R^{W17-1} is H or R^{A12}, and R^{W17-2} is H or R^{A12}.

W¹⁸ is selected from the group consisting of —C(R^{W18-1}R^{W18-2})—, —N(R^{W18-2})—, —C(R^{W18-1}R^{W18-1})N(R^{W18-2})—, —N(R^{W18-1})C(R^{W18-1}R^{W18-2})—, —C(R^{W18-1})═N—, —N═C(R^{W18-1})—, —O—, —C(R^{W18-1}R^{W18-1})O—, —OC(R^{W18-1}R^{W18-2})—, —S—, —C(R^{W18-1}R^{W18-1})S—, —SC(R^{W18-1}R^{W18-2})—, —C(R^{W18-1}R^{W18-1})C(R^{W18-1}R^{W18-2})—, and —CR^{W18-1}═CR^{W18-1}.

wherein R^{W8}-1 is H or R^{A12}, and R^{W18-2} is H or R^{A12}.

W¹⁹, independently at each occurrence, is CR^{W19} or N, wherein R^{W19} is H or R^{A12}. R^{W16} is hydrogen or R^{A12}, or R^{W16} and R^{W17-2} are taken together to form a double bond between the carbon atom bearing R^{W16} and the atom bearing R^{W17-2}, or R^{W16} and R^{W18-2} are taken together to form a double bond between the carbon atom bearing R^{W16} and the atom bearing R^{W18-2};

In some embodiments, (A¹²-1) is selected from the group consisting of

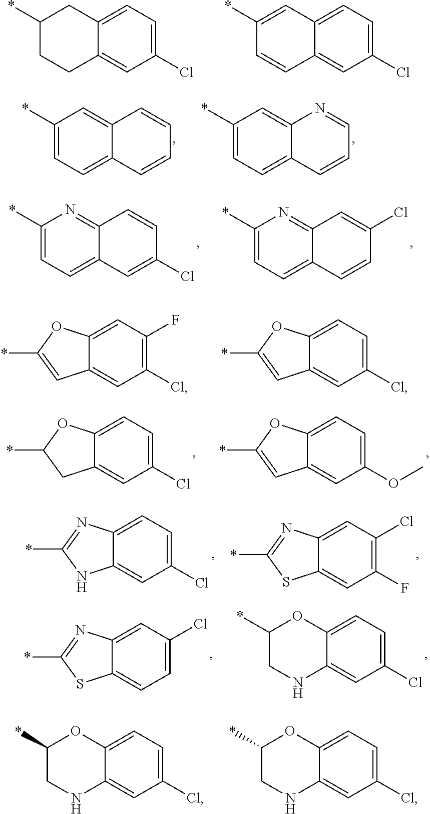

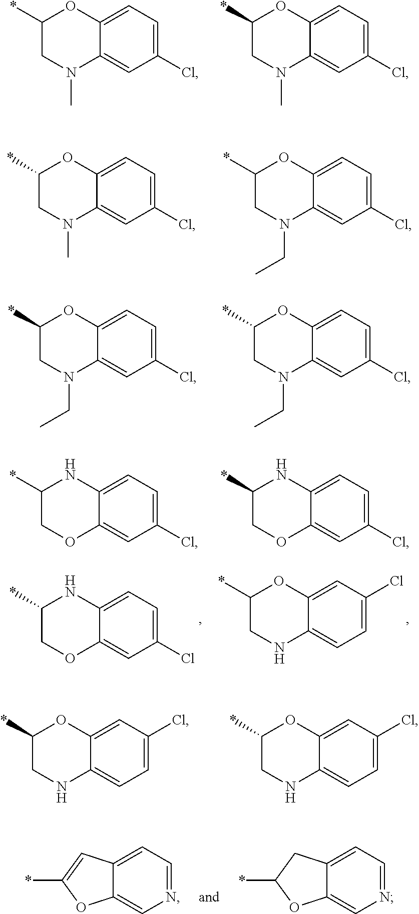

wherein * represents the attachment point to the remainder of the molecule. In some embodiments (A¹²-1) is selected from the group consisting of

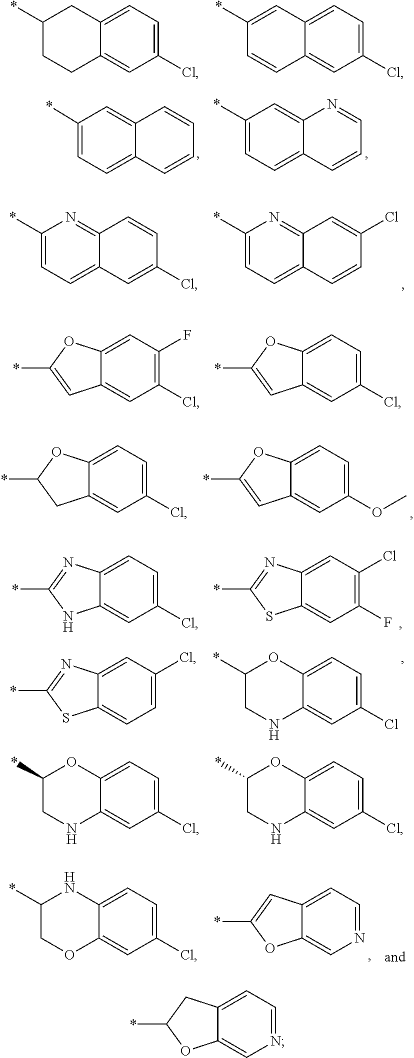

wherein * represents the attachment point to the remainder of the molecule. In some embodiments, (A¹²-1) is

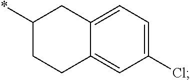 [SMILES: *C1CCc2cc(Cl)ccc2C1]

wherein * represents the attachment point to the remainder of the molecule. In some embodiments, (A¹²-1) is

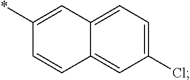 [SMILES: *c1ccc2cc(Cl)ccc2c1]

wherein * represents the attachment point to the remainder of the molecule. In some embodiments, (A¹²-1) is

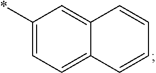 [SMILES: *c1ccc2ccccc2c1]

wherein * represents the attachment point to the remainder of the molecule. In some embodiments, (A¹²-1) is

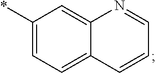 [SMILES: *c1ccc2cccnc2c1]

wherein * represents the attachment point to the remainder of the molecule. In some embodiments, (A¹²-1) is

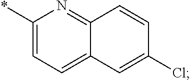 [SMILES: *c1ccc2cc(Cl)ccc2n1]

wherein * represents the attachment point to the remainder of the molecule. In some embodiments, (A¹²-1) is

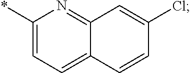 [SMILES: *c1ccc2ccc(Cl)cc2n1]

wherein * represents the attachment point to the remainder of the molecule. In some embodiments, (A¹²-1) is

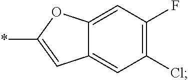 [SMILES: *c1cc2cc(Cl)c(F)cc2o1]

wherein * represents the attachment point to the remainder of the molecule. In some embodiments, (A¹²-1) is

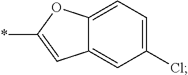 [SMILES: *c1cc2cc(Cl)ccc2o1]

wherein * represents the attachment point to the remainder of the molecule. In some embodiments, (A¹²-1) is

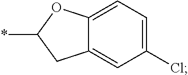 [SMILES: *C1Cc2cc(Cl)ccc2O1]

wherein * represents the attachment point to the remainder of the molecule. In some embodiments, (A¹²-1) is

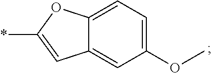 [SMILES: *c1cc2cc(OC)ccc2o1]

wherein * represents the attachment point to the remainder of the molecule. In some embodiments, (A¹²-1) is

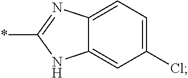 [SMILES: *c1nc2ccc(Cl)cc2[nH]1]

wherein * represents the attachment point to the remainder of the molecule. In some embodiments, (A¹² 1) is

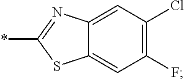 [SMILES: *c1nc2cc(Cl)c(F)cc2s1]

wherein * represents the attachment point to the remainder of the molecule. In some embodiments, (A¹²-1) is

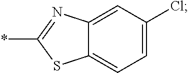 [SMILES: *c1nc2cc(Cl)ccc2s1]

wherein * represents the attachment point to the remainder of the molecule. In some embodiments, (A¹²-1) is

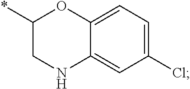 [SMILES: *C1CNc2cc(Cl)ccc2O1]

Wherein * represents the attachment point to the remainder of the molecule. In some embodiments, (A¹²-1) is

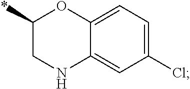 [SMILES: *[C@@H]1CNc2cc(Cl)ccc2O1]

wherein * represents the attachment point to the remainder of the molecule. In some embodiments, (A¹²-1) is

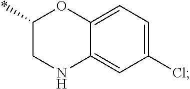 [SMILES: *[C@H]1CNc2cc(Cl)ccc2O1]

wherein * represents the attachment point to the remainder of the molecule. In some embodiments, (A¹²-1) is

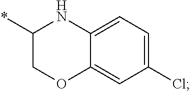 [SMILES: *C1COc2cc(Cl)ccc2N1]

wherein * represents the attachment point to the remainder of the molecule. In some embodiments, (A¹²-1) is

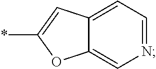 [SMILES: *c1cc2ccncc2o1]

wherein * represents the attachment point to the remainder of the molecule. In some embodiments, (A¹²-1) is

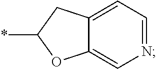 [SMILES: *C1Cc2ccncc2O1]

wherein * represents the attachment point to the remainder of the molecule.

In some embodiments of the compounds of formula (F-1), or the salts thereof, A¹² is C₆-C₁₄ aryl optionally substituted with 1, 2, 3, 4, 5, 6, 7, 8, or 9 R^{A12} substituents. In some embodiments, A¹² is selected from the group consisting of

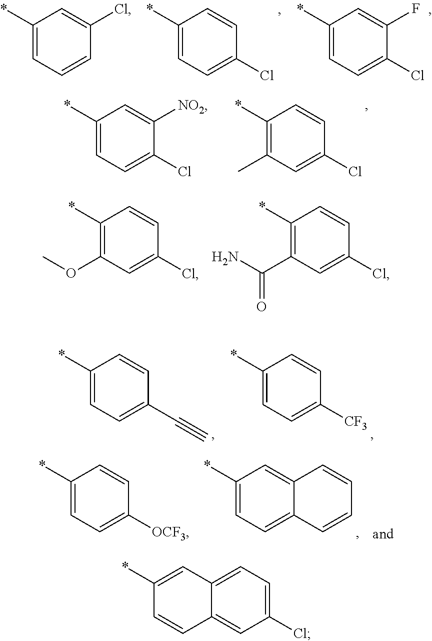

wherein * represents the attachment point to the remainder of the molecule. In some embodiments, A¹² is selected from the group consisting of

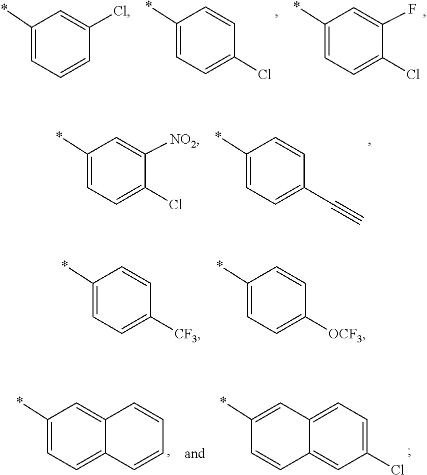

wherein * represents the attachment point to the remainder of the molecule. In some embodiments, A¹² is

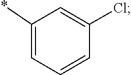 [SMILES: *c1cccc(Cl)c1]

wherein * represents the attachment point to the remainder of the molecule. In some embodiments, A¹² is

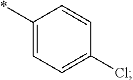 [SMILES: *c1ccc(Cl)cc1]

wherein * represents the attachment point to the remainder of the molecule. In some embodiments, A¹² is

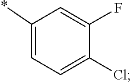 [SMILES: *c1ccc(Cl)c(F)c1]

wherein * represents the attachment point to the remainder of the molecule. In some embodiments, A¹² is

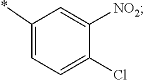 [SMILES: *c1ccc(Cl)c([N+](=O)[O-])c1]

wherein * represents the attachment point to the remainder of the molecule. In some embodiments, A¹² is

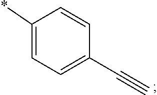 [SMILES: *c1ccc(C#C)cc1]

wherein * represents the attachment point to the remainder of the molecule. In some embodiments, A¹² is

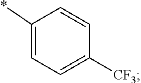 [SMILES: *c1ccc(C(F)(F)F)cc1]

wherein * represents the attachment point to the remainder of the molecule. In some embodiments, A¹² is

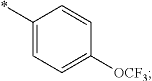 [SMILES: *c1ccc(OC(F)(F)F)cc1]

wherein * represents the attachment point to the remainder of the molecule. In some embodiments, A is

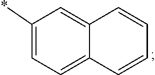 [SMILES: *c1ccc2ccccc2c1]

wherein * represents the attachment point to the remainder of the molecule. In some embodiments, A¹² is

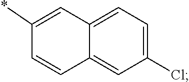 [SMILES: *c1ccc2cc(Cl)ccc2c1]

wherein * represents the attachment point to the remainder of the molecule.

In some embodiments of the compounds of formula (F-1), or the salts thereof, A¹² is 5-14 membered heteroaryl optionally substituted with 1, 2, 3, 4, 5, 6, 7, 8, or 9 R^{A12} substituents. In some embodiments, A¹² is selected from the group consisting of

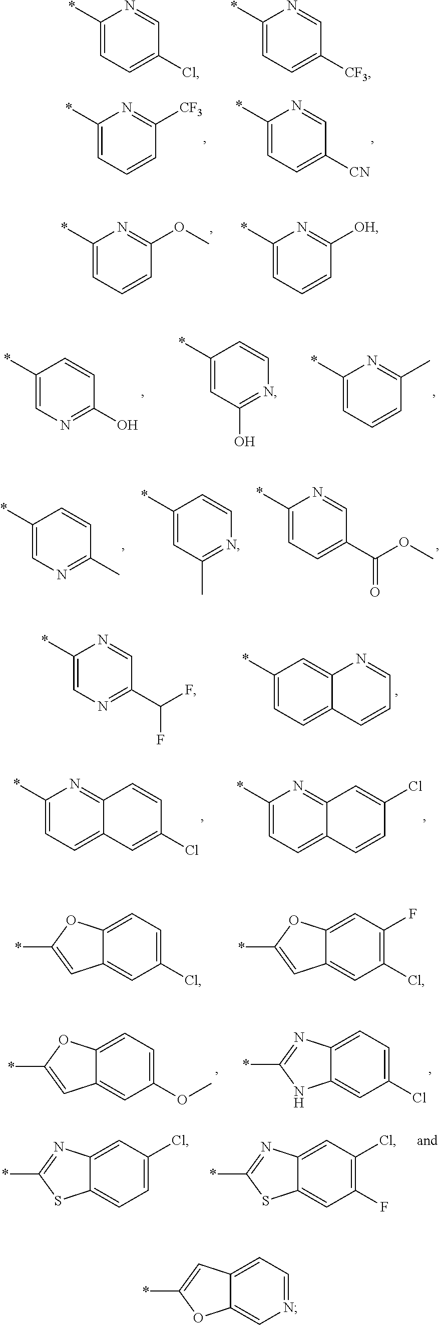

wherein * represents the attachment point to the remainder of the molecule. In some embodiments, A¹² is selected from the group consisting of

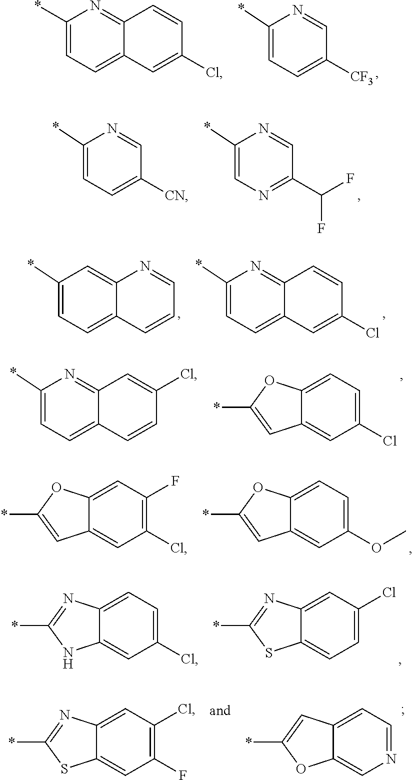

wherein * represents the attachment point to the remainder of the molecule. In some embodiments, A¹² is

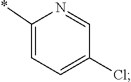 [SMILES: *c1ccc(Cl)cn1]

wherein * represents the attachment point to the remainder of the molecule. In some embodiments, A¹² is

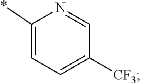 [SMILES: *c1ccc(C(F)(F)F)cn1]

wherein * represents the attachment point to the remainder of the molecule. In some embodiments, A¹² is

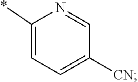 [SMILES: *c1ccc(C#N)cn1]

wherein * represents the attachment point to the remainder of the molecule. In some embodiments, A¹² is

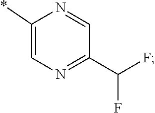 [SMILES: *c1cnc(C(F)F)cn1]

wherein * represents the attachment point to the remainder of the molecule. In some embodiments, A¹² is

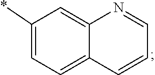 [SMILES: *c1ccc2cccnc2c1]

wherein * represents the attachment point to the remainder of the molecule. In some embodiments, A¹² is

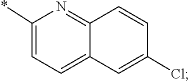 [SMILES: *c1ccc2cc(Cl)ccc2n1]

wherein * represents the attachment point to the remainder of the molecule. In some embodiments, A¹² is

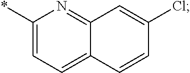 [SMILES: *c1ccc2ccc(Cl)cc2n1]

wherein * represents the attachment point to the remainder of the molecule. In some embodiments, A¹² is

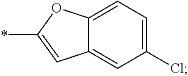 [SMILES: *c1cc2cc(Cl)ccc2o1]

wherein * represents the attachment point to the remainder of the molecule. In some embodiments, A¹² is

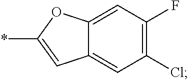 [SMILES: *c1cc2cc(Cl)c(F)cc2o1]

wherein * represents the attachment point to the remainder of the molecule. In some embodiments, A¹² is

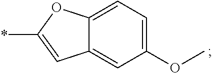 [SMILES: *c1cc2cc(OC)ccc2o1]

wherein * represents the attachment point to the remainder of the molecule. In some embodiments, A¹² is

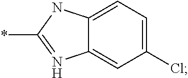 [SMILES: *c1nc2cc(Cl)ccc2[nH]1]

wherein * represents the attachment point to the remainder of the molecule. In some embodiments, A¹² is

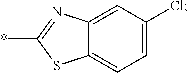 [SMILES: *c1nc2cc(Cl)ccc2s1]

wherein * represents the attachment point to the remainder of the molecule. In some embodiments, A¹² is

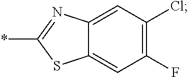 [SMILES: *c1nc2cc(Cl)c(F)cc2s1]

wherein * represents the attachment point to the remainder of the molecule. In some embodiments, A¹² is

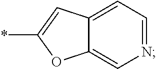 [SMILES: *c1cc2ccncc2o1]

wherein * represents the attachment point to the remainder of the molecule.

In ninth aspect, provided is a compound of formula (III):

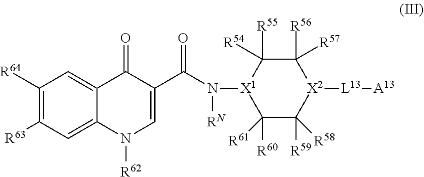

or a salt thereof,
wherein:
X¹ is N or CR^{X1};
X² is N or CR^{X2};
when present, R^{X1} is selected from the group consisting of hydrogen, C₁-C₆ alkyl, C₁-C₆ haloalkyl, —C(O)OH, —C(O)O(C₁-C₆ alkyl), —C(O)O(C₁-C₆ haloalkyl), and halogen;
when present, R² is selected from the group consisting of hydrogen, C₁-C₆ alkyl, C₁-C₆ haloalkyl, —C(O)OH, —C(O)O(C₁-C₆ alkyl), —C(O)O(C₁-C₆ haloalkyl), and halogen;
R⁵⁴, R⁵⁵, R⁵⁶, R⁵⁷, R⁵⁸, R⁵⁹, R⁶⁰, and R⁶¹, independently from each other, are selected from the group consisting of hydrogen, C₁-C₆ alkyl, C₁-C₆ haloalkyl, —C(O)OH, —C(O)O(C₁-C₆ alkyl), —C(O)O(C₁-C₆ haloalkyl), and halogen;
or, one of R⁵⁴, R⁵⁵, R⁵⁶, R⁵⁷, R⁵⁸, R⁵⁹, R⁶⁰, and R⁶¹, and another one of R⁵⁴, R⁵⁵, R⁵⁶, R⁵⁷, R⁵⁸, R⁵⁹, R⁶⁰, and R⁶¹, are taken together to form a C₁-C₆ alkylene moiety;
or, two geminal substituents selected from the group consisting of R⁵⁴, R⁵⁵, R⁵⁶, R⁵⁷, R⁵⁸, R⁵⁹, R⁶⁰, and R⁶¹ are taken together to form an oxo group;
or, two of R⁵⁴, R⁵⁵, R⁵⁶, R⁵⁷, R⁵⁸, R⁵⁹, R⁶⁰, R⁶¹, R^{X1} when present, and R^{X2}, when present, are taken together to form a C₁-C₆ alkylene moiety;
R⁶³ and R⁶⁴, independently from each other, are selected from the group consisting of hydrogen, halogen, NO₂, C₁-C₆ alkyl, C₂-C₆ alkenyl, C₂-C₆ alkynyl, C₁-C₆ haloalkyl, —OH, —O(C₁-C₆ alkyl), —O(C₁-C₆ haloalkyl), —SH, —S(C₁-C₆ alkyl), —S(C₁-C₆ haloalkyl), —NH₂, —NH(C₁-C₆ alkyl), —NH(C₁-C₆ haloalkyl), —N(C₁-C₆ alkyl)₂, —N(C₁-C₆ haloalkyl)₂, —NR^{B-a}R^{B-b}, —CN, —C(O)OH, —C(O)O(C₁-C₆ alkyl), —C(O)O(C₁-C₆ haloalkyl), —C(O)NH₂, —C(O)NH(C₁-C₆ alkyl), —C(O)NH(C₁-C₆ haloalkyl), —C(O)N(C₁-C₆ alkyl)₂, —C(O)N(C₁-C₆ haloalkyl)₂, —C(O)NR^{B-a}R^{B-b}, —S(O)₂OH, —S(O)₂O(C₁-C₆ alkyl), —S(O)₂O(C₁-C₆ haloalkyl), —S(O)₂NH₂, —S(O)₂NH(C₁-C₆ alkyl), —S(O)₂NH(C₁-C₆ haloalkyl), —S(O)₂N(C₁-C₆ alkyl)₂, —S(O)₂N(C₁-C₆ haloalkyl)₂, —S(O)₂NR^{B-a}R^{B-b}, —OC(O)H, —OC(O)(C₁-C₆ alkyl), —OC(O)(C₁-C₆ haloalkyl), —N(H)C(O)H, —N(H)C(O)(C₁-C₆ alkyl), —N(H)C(O)(C₁-C₆ haloalkyl), —N(C₁-C₆ alkyl)C(O)H, —N(C₁-C₆ alkyl)C(O)(C₁-C₆ alkyl), —N(C₁-C₆ alkyl)C(O)(C₁-C₆ haloalkyl), —N(C₁-C₆ haloalkyl)C(O)H, —N(C₁-C₆ haloalkyl)C(O)(C₁-C₆ alkyl), —N(C₁-C₆ haloalkyl)C(O)(C₁-C₆ haloalkyl), —OS(O)₂(C₁-C₆ alkyl), —OS(O)₂(C₁-C₆ haloalkyl), —N(H)S(O)₂(C₁-C₆ alkyl), —N(H)S(O)₂(C₁-C₆ haloalkyl), —N(C₁-C₆ alkyl)S(O)₂(C₁-C₆ alkyl), —N(C₁-C₆ alkyl)S(O)₂(C₁-C₆ haloalkyl), —N(C₁-C₆ haloalkyl)S(O)₂(C₁-C₆ alkyl), and —N(C₁-C₆ haloalkyl)S(O)₂(C₁-C₆ haloalkyl);
   wherein R^{B-a} and R^{B-b} are taken together with the nitrogen atom to which they are attached to form a 3-10 membered heterocycle;
R⁶² is selected from the group consisting of halogen, NO₂, C₁-C₆ alkyl, C₂-C₆ alkenyl, C₂-C₆ alkynyl, —(C₁-C₆ alkylene)-(C₆-C₁₄ aryl optionally substituted with 1, 2, 3, 4, 5, 6, 7, 8, or 9 R^{A13} substituents), —(C₁-C₆ alkylene)-(5-14 membered heteroaryl optionally substituted with 1, 2, 3, 4, 5, 6, 7, 8, or 9 R^{A13} substituents), C₁-C₆ haloalkyl, —OH, —O(C₁-C₆ alkyl), —O(C₁-C₆ haloalkyl), —(C₁-C₆ alkylene)-OH, —(C₁-C₆ alkylene)-O—(C₁-C₆ alkyl), —(C₁-C₆ alkylene)-O—(C₁-C₆ haloalkyl), —SH, —S(C₁-C₆ alkyl), —S(C₁-C₆ haloalkyl), —NH₂, —NH(C₁-C₆ alkyl), —NH(C₁-C₆ haloalkyl), —N(C₁-C₆ alkyl)₂, —N(C₁-C₆ haloalkyl)₂, —CN, —C(O)OH, —C(O)O(C₁-C₆ alkyl), —C(O)O(C₁-C₆ haloalkyl), —C(O)NH₂, —C(O)NH(C₁-C₆ alkyl), —C(O)NH(C₁-C₆ haloalkyl), —C(O)N(C₁-C₆ alkyl)₂, —C(O)N(C₁-C₆ haloalkyl)₂, —C(O)NR^{62-a}R^{62-b}, —S(O)₂H, —S(O)₂(C₁-C₆ alkyl), —S(O)₂O(C₁-C₆ haloalkyl), —S(O)₂NH₂, —S(O)₂NH(C₁-C₆ alkyl), —S(O)₂NH(C₁-C₆ haloalkyl), —S(O)₂N(C₁-C₆ alkyl)₂, —S(O)₂N(C₁-C₆ haloalkyl)₂, —S(O)₂NR^{62-a}R^{62-b}, —OC(O)H, —OC(O)(C₁-C₆ alkyl), —OC(O)(C₁-C₆ haloalkyl), —N(H)C(O)H, —N(H)C(O)(C₁-C₆ alkyl), —N(H)C(O)(C₁-C₆ haloalkyl), —N(C₁-C₆ alkyl)C(O)H, —N(C₁-C₆ alkyl)C(O)(C₁-C₆ alkyl), —N(C₁-C₆ alkyl)C(O)(C₁-C₆ haloalkyl), —N(C₁-C₆ haloalkyl)C(O)H, —N(C₁-C₆ haloalkyl)C(O)(C₁-C₆ alkyl), —N(C₁-C₆ haloalkyl)C(O)(C₁-C₆ haloalkyl), —OS(O)₂(C₁-C₆ alkyl), —OS(O)₂(C₁-C₆ haloalkyl), —N(H)S(O)₂(C₁-C₆ alkyl), —N(H)S(O)₂(C₁-C₆ haloalkyl), —N(C₁-C₆ alkyl)S(O)₂(C₁-C₆ alkyl), —N(C₁-C₆ alkyl)S(O)₂(C₁-C₆ haloalkyl), —N(C₁-C₆ haloalkyl)S(O)₂(C₁-C₆ alkyl), and —N(C₁-C₆ haloalkyl)S(O)₂(C₁-C₆ haloalkyl);
wherein R^{62-a} and R^{62-b} are taken together with the nitrogen atom to which they are attached to form a 3-10 membered heterocycle;
L¹³ is a linker selected from the group consisting of @¹³—C₁-C₆ alkylene-#¹³, @¹³—NR^{N}—(C₁-C₆ alkylene)-#¹³, @¹³—NR^{N}—NR^{N}—(C₁-C₆ alkylene)-#¹³ @13—CH₂—NR^{N}—(C₁-C₆ alkylene)-#¹³, @¹³—CH₂—NR^{N}—NR^{N}—(C₁-C₆ alkylene)-#¹³, @¹³—NR^{N}—(C₁-C₆ alkylene)-O-#¹³, @¹³—NR^{N}—NR^{N}—(C₁-C₆ alkylene)-O-#¹³, @¹³—CH₂—NR^{N}—(C₁-C₆ alkylene)-O-#¹³, @¹³—CH₂—NR^{N}—NR^{N}—(C₁-C₆ alkylene)-O-#¹³, and @¹³—(C₁-C₆ alkylene)-O-#¹³;
   wherein @¹³ represents the attachment point to X² and #¹³ represents the attachment point to A¹³;
   the C₁-C₆ alkylene moiety of each of the @¹³—C₁-C₆ alkylene-#¹³, @¹³—NR^{N}—(C₁-C₆ alkylene)-#¹³, @¹³—NR^{N}—NR^{N}—(C₁-C₆ alkylene)-#¹³, @¹³—CH₂—NR^{N}—(C₁-C₆ alkylene)-#¹³, @¹³—CH₂—NR^{N}—NR^{N}—(C₁-C₆ alkylene)-#¹³, @¹³—NR^{N}—(C₁-C₆ alkylene)-O-#¹³, @¹³—NR^{N}—NR^{N}—(C₁-C₆ alkylene)-O-#¹³, @¹³—CH₂—NR^{N}—(C₁-C₆ alkylene)-O-#¹³, @¹³—CH₂—NR^{N}—NR^{N}—(C₁-C₆ alkylene)-O-#¹³, and @¹³—(C₁-C₆ alkylene)-O-#¹³ is optionally substituted with 1 to 12 R⁶⁶;
   R^{N}, independently at each occurrence, is selected from the group consisting of hydrogen, C₁-C₆ alkyl, and C₁-C₆ haloalkyl,
   R⁶⁶, independently at each occurrence, is selected from the group consisting of oxo, halogen, C₁-C₆ alkyl, C₂-C₆ alkenyl, C₂-C₆ alkynyl, C₁-C₆ haloalkyl, —OH, —O(C₁-C₆ alkyl), —O(C₁-C₆ haloalkyl), —SH, —S(C₁-C₆ alkyl), —S(C₁-C₆ haloalkyl), —NH₂, —NH(C₁-C₆ alkyl), —NH(C₁-C₆ haloalkyl), —N(C₁-C₆ alkyl)₂, —N(C₁-C₆ haloalkyl)₂, —NR^{B-a}R^{B-b}, —CN, —C(O)OH, —C(O)O(C₁-C₆ alkyl), —C(O)O(C₁-C₆ haloalkyl), —C(O)NH₂, —C(O)NH(C₁-C₆ alkyl), —C(O)NH(C₁-C₆ haloalkyl), —C(O)N(C₁-C₆ alkyl)₂, —C(O)N(C₁-C₆ haloalkyl)₂, —C(O)NR^{B-a}R^{B-b}, —S(O)₂OH, —S(O)₂₀(C₁-C₆ alkyl), —S(O)₂₀(C₁-C₆ haloalkyl), —S(O)₂NH₂, —S(O)₂NH(C₁-C₆ alkyl), —S(O)₂NH(C₁-C₆ haloalkyl), —S(O)₂N(C₁-C₆ alkyl)₂, —S(O)₂N(C₁-C₆ haloalkyl)₂, —S(O)₂NR^{B-a}R^{B-b}, —OC(O)H, —OC(O)(C₁-C₆ alkyl), —OC(O)(C₁-C₆ haloalkyl), —N(H)C(O)H, —N(H)C(O)(C₁-C₆ alkyl), —N(H)C(O)(C₁-C₆ haloalkyl), —N(C₁-C₆ alkyl)C(O)H, —N(C₁-C₆ alkyl)C(O)(C₁-C₆ alkyl), —N(C₁-C₆ alkyl)C(O)(C₁-C₆ haloalkyl), —N(C₁-C₆ haloalkyl)C(O)H, —N(C₁-C₆ haloalkyl)C(O)(C₁-C₆ alkyl), —N(C₁-C₆ haloalkyl)C(O)(C₁-C₆ haloalkyl), —OS(O)₂(C₁-C₆ alkyl), —OS(O)₂(C₁-C₆ haloalkyl), —N(H)S(O)₂(C₁-C₆ alkyl), —N(H)S(O)₂(C₁-C₆ haloalkyl), —N(C₁-C₆ alkyl)S(O)₂(C₁-C₆ alkyl), —N(C₁-C₆ alkyl)S(O)₂(C₁-C₆ haloalkyl), —N(C₁-C₆ haloalkyl)S(O)₂(C₁-C₆ alkyl), and —N(C₁-C₆ haloalkyl)S(O)₂(C₁-C₆ haloalkyl);
A¹³ is selected from the group consisting of:
   a substituent of formula (A¹³-1)

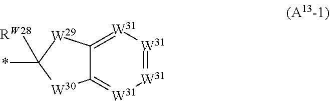

W²⁹ is selected from the group consisting of —C(R^{W29-1}R^{W29-2})—, —N(R^{W29-2})—, —C(R^{W29-1}R^{W29-1})N(R^{W29-2})—, —N(R^{W29-1})C(R^{W29-1}R^{W29-2})—, —C(R^{W29-1})═N—, —N═C(R^{W29-1})—, —O—, —C(R^{W29-1}R^{W29-1})O—, —OC(R^{W29-1}R^{W29-2})—, —O—, —C(R^{W29-1}R^{W29-1})S—, —SC(R^{W29-1}R^{W29-2})—, —C(R^{W29-1}R^{W29-1})C(R^{W29-1}R^{W29-2})—, and —CR^{W29-1}═CR^{W29-1}—,
   wherein R^{W29-1} is H or R^{A1}, and R^{W29-2} is H or R^{A13};
W³⁰ is selected from the group consisting of —C(R^{W30-1}R^{W30-2}), N(R^{W30-2}—, —C(R^{W30-1}R^{W30-1})N(R^{W30-2})—, —N(R^{W30-1})C(R^{W30-1}R^{W30-2})—, —C(R^{W30-1})═N—, —N═C(R^{W30-1})—, —O—, —C(R^{W30-1}R^{W30-1-1})O—, —OC(R^{W30-1}R^{W30-2}), —S—, —C(R^{W30-1}R^{W30-1})S—, —SC(R^{W30-1}R^{W30-2})—, —C(R^{W30-1}R^{W30-1})C(R^{W30-1}R^{W30-2})—, and —CR^{W30-1}═CR^{W30-1}—,
   wherein R^{W30-1} is H or R^{A}, and R^{W30-2} is H or R^{A13};
W³¹, independently at each occurrence, is CR^{W31} or N, wherein R^{W31} is H or R^{A13}.
R^{W28} is hydrogen or R^{A13}, or R^{W28} and R^{W29-2} are taken together to form a double bond between the carbon atom bearing R^{W28} and the atom bearing R^{W29-2}, or R^{W28} and R^{W30-2} are taken together to form a double bond between the carbon atom bearing R^{W28} and the atom bearing R^{W30-2};
C₆-C₁₄ aryl optionally substituted with 1, 2, 3, 4, 5, 6, 7, 8, or 9 R^{A13} substituents; and
5-14 membered heteroaryl optionally substituted with 1, 2, 3, 4, 5, 6, 7, 8, or 9 R^{A13} substituents;
R^{A13}, independently at each occurrence, is selected from the group consisting of halogen, NO₂, C₁-C₆ alkyl, C₂-C₆ alkenyl, C₂-C₆ alkynyl, C₁-C₆ haloalkyl, —OH, —O(C₁-C₆ alkyl), —O(C₁-C₆ haloalkyl), —SH, —S(C₁-C₆ alkyl), —S(C₁-C₆ haloalkyl), —NH₂, —NH(C₁-C₆ alkyl), —NH(C₁-C₆ haloalkyl), —N(C₁-C₆ alkyl)₂, —N(C₁-C₆ haloalkyl)₂, —NR^{A13-a}R^{A13-b}, —CN, —C(O)OH, —C(O)O(C₁-C₆ alkyl), —C(O)O(C₁-C₆ haloalkyl), —C(O)NH₂, —C(O)NH(C₁-C₆ alkyl), —C(O)NH(C₁-C₆ haloalkyl), —C(O)N(C₁-C₆ alkyl)₂, —C(O)N(C₁-C₆ haloalkyl)₂, —C(O)NR^{A13-a}R^{A13-b}, —S(O)₂H, —S(O)₂(C₁-C₆ alkyl), —S(O)₂O(C₁-C₆ haloalkyl), —S(O)₂NH₂, —S(O)₂NH(C₁-C₆ alkyl), —S(O)₂NH(C₁-C₆ haloalkyl), —S(O)₂N(C₁-C₆ alkyl)₂, —S(O)₂N(C₁-C₆ haloalkyl)₂, —S(O)₂NR^{A13-a}R^{A13-b}, —OC(O)H, —OC(O)(C₁-C₆ alkyl), —OC(O)(C₁-C₆ haloalkyl), —N(H)C(O)H, —N(H)C(O)(C₁-C₆ alkyl), —N(H)C(O)(C₁-C₆ haloalkyl), —N(C₁-C₆ alkyl)C(O)H, —N(C₁-C₆ alkyl)C(O)(C₁-C₆ alkyl), —N(C₁-C₆ alkyl)C(O)(C₁-C₆ haloalkyl), —N(C₁-C₆ haloalkyl)C(O)H, —N(C₁-C₆ haloalkyl)C(O)(C₁-C₆ alkyl), —N(C₁-C₆ haloalkyl)C(O)(C₁-C₆ haloalkyl), —OS(O)₂(C₁-C₆ alkyl), —OS(O)₂(C₁-C₆ haloalkyl), —N(H)S(O)₂(C₁-C₆ alkyl), —N(H)S(O)₂(C₁-C₆ haloalkyl), —N(C₁-C₆ alkyl)S(O)₂(C₁-C₆ alkyl), —N(C₁-C₆ alkyl)S(O)₂(C₁-C₆ haloalkyl), —N(C₁-C₆ haloalkyl)S(O)₂(C₁-C₆ alkyl), and —N(C₁-C₆ haloalkyl)S(O)₂(C₁-C₆ haloalkyl);
   wherein R^{A13-a} and R^{A13-b} are taken together with the nitrogen atom to which they are attached to form a 3-10 membered heterocycle;
provided that when X² is N, then L¹³ is a linker selected from the group consisting of @¹³—C₁-C₆ alkylene-#¹³, @¹³—NR^{N}—(C₁-C₆ alkylene)#^{13 @13}—NR^{N}—(C₁-C₆ alkylene)-O-#¹³, and @¹³—(C₁-C₆ alkylene)-O-#¹³; and further provided that when X¹ is CH, X² is N, R⁶² is methyl, and L¹³ is @¹³—CH₂-#¹³, then A¹³ is then A³ is (A¹³-1), C₆-C₁₄ aryl optionally substituted with 1, 2, 3, 4, 5, 6, 7, 8, or 9 R^{A13} substituents, or 5-14 membered heteroaryl substituted with 1, 2, 3, 4, 5, 6, 7, 8, or 9 R^{A13} substituents.

In some embodiments, the compound of formula (III), or the salt thereof, is a compound of formula (IV):

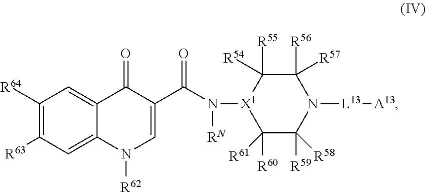

or a salt thereof,
wherein R⁵⁴, R⁵⁵, R⁵⁶, R⁵⁷, R⁵⁸, R⁵⁹, R⁶⁰, R⁶¹, R⁶², R⁶³, R⁶⁴, X¹, L¹³, and A¹³ are as defined in compounds of formula (III); provided that L¹³ is a linker selected from the group consisting of @¹³—C₁-C₆ alkylene-#¹³, @¹³—NR^{N}—(C₁-C₆ alkylene)-#¹³, @¹³—NR^{N}—(C₁-C₆ alkylene)-O-#¹³, and @¹³—(C₁-C₆ alkylene)-O-#¹³; and further provided that when X¹ is CH, R⁶² is methyl, and L¹³ is @¹³—CH₂-#¹³, then A¹³ is then A¹³ is (A¹³-1), C₆-C₁₄ aryl optionally substituted with 1, 2, 3, 4, 5, 6, 7, 8, or 9 R^{A13} substituents, or 5-14 membered heteroaryl substituted with 1, 2, 3, 4, 5, 6, 7, 8, or 9 R^{A13} substituents.

In some embodiments, the compound of formula (III), or the salt thereof, is a compound of formula (V):

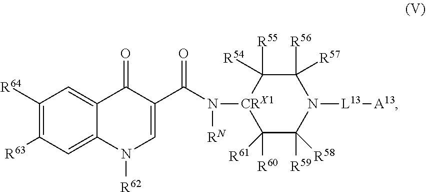

or a salt thereof,
wherein R⁵⁴, R⁵⁵, R⁵⁶, R⁵⁷, R⁵⁸, R⁵⁹, R⁶⁰, R⁶¹, R⁶², R⁶³, R^{IX}, R^{X}, L¹³, and A¹³ are as defined in compounds of formula (III); provided that L¹³ is a linker selected from the group consisting of @¹³—C₁-C₆ alkylene-#¹³, @¹³—NR^{N}—(C₁-C₆ alkylene)-#¹³, @¹³—NR^{N}—(C₁-C₆ alkylene)-O-#¹³, and @¹³—(C₁-C₆ alkylene)-O-#¹³; and further provided that when Rx is H, R⁶² is methyl, and L¹³ is @¹³—CH₂-#¹³, then A¹³ is then A¹³ is (A¹³-1), C₆-C₁₄ aryl optionally substituted with 1, 2, 3, 4, 5, 6, 7, 8, or 9 R^{A13} substituents, or 5-14 membered heteroaryl substituted with 1, 2, 3, 4, 5, 6, 7, 8, or 9 R^{A13} substituents.

In some embodiments, the compound of formula (III), or the salt thereof, is a compound of formula (VI):

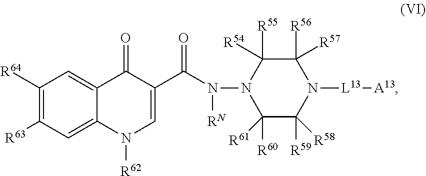

or a salt thereof,
wherein R⁵⁴, R⁵⁵, R⁵⁶, R⁵⁷, R⁵⁸, R⁵⁹, R⁶⁰, R⁶¹, R⁶², R⁶³, R⁶⁴, X¹, L¹³, and A¹³ are as defined in compounds of formula (III); provided that L¹³ is a linker selected from the group consisting of @¹³—C₁-C₆ alkylene-#¹³, @¹³—NR^{N}—(C₁-C₆ alkylene)-#¹³, @¹³—NR^{N}—(C₁-C₆ alkylene)-O-#¹³, and @¹³—(C₁-C₆ alkylene)-O-#¹³.

In some embodiments, the compound of formula (III), or the salt thereof, is a compound of formula (VII):

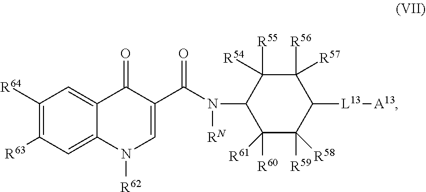

or a salt thereof,
wherein R⁵⁴, R⁵⁵, R⁵⁶, R⁵⁷, R⁵⁸, R⁵⁹, R⁶⁰, R⁶¹, R⁶², R⁶³, R⁶⁴, L¹³, and A¹³ are as defined in compounds of formula (III).

In some embodiments, the compound of formula (III), or the salt thereof, is a compound of formula (VIII):

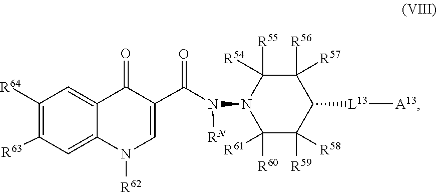

or a salt thereof,
wherein R⁵⁴, R⁵⁵, R⁵⁶, R⁵⁷, R⁵⁸, R⁵⁹, R⁶⁰, R⁶¹, R⁶², R⁶³, R, L¹³, and A¹³ are as defined in compounds of formula (III).

In some embodiments, the compound of formula (III), or the salt thereof, is a compound of formula (IX):

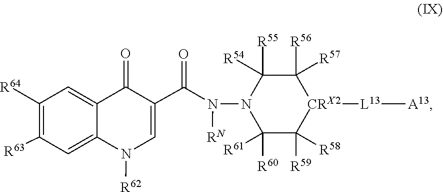

or a salt thereof,
wherein R⁵⁴, R⁵⁵, R⁵⁶, R⁵⁷, R⁵⁸, R⁵⁹, R⁶⁰, R⁶¹, R⁶², R⁶³, R⁶⁴, R^{X2}, L¹³, and A¹³ are as defined in compounds of formula (III).

In some embodiments, the compound of formula (III), or the salt thereof, is a compound of formula (X):

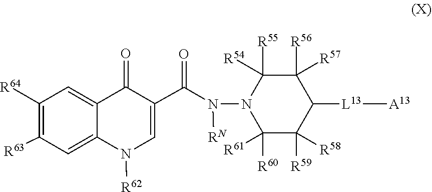

or a salt thereof,
wherein R⁵⁴, R⁵⁵, R⁵⁶, R⁵⁷, R⁵⁸, R⁵⁹, R⁶⁰, R⁶¹, R⁶², R⁶³, R⁶⁴, L¹³, and A¹³ are as defined in compounds of formula (III).

In some embodiments of the compounds of formulae (III), (IV), (V), (VI), (VII), (VIII), (IX), and (X), or the salts thereof, R⁵⁴, R⁵⁵, R⁵⁶, R⁵⁷, R⁵⁸, R⁵⁹, R⁶⁰, and R⁶¹ are each hydrogen. In such embodiments, the compound of formula (III), or the salt thereof, is a compound of formula (XI), or a salt thereof. In such embodiments, the compound of formula (IV), or the salt thereof, is a compound of formula (XII), or a salt thereof. In such embodiments, the compound of formula (V), or the salt thereof, is a compound of formula (XIIII), or a salt thereof. In such embodiments, the compound of formula (VI), or the salt thereof, is a compound of formula (XIV), or a salt thereof. In such embodiments, the compound of formula (VII), or the salt thereof, is a compound of formula (XV), or a salt thereof. In such embodiments, the compound of formula (VIII), or the salt thereof, is a compound of formula (XVI), or a salt thereof. In such embodiments, the compound of formula (IX), or the salt thereof, is a compound of formula (XVII), or a salt thereof. In such embodiments, the compound of formula (X), or the salt thereof, is a compound of formula (XVIII), or a salt thereof.

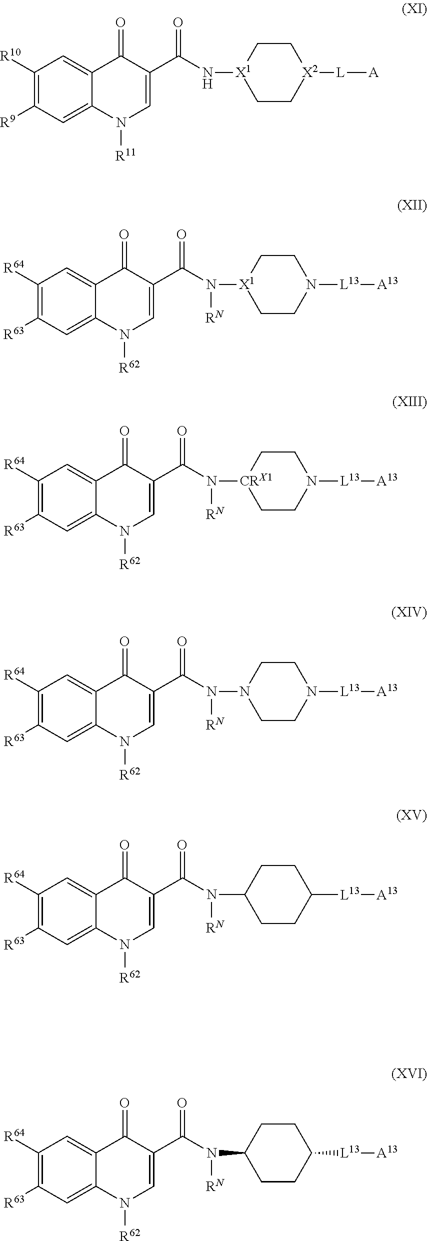

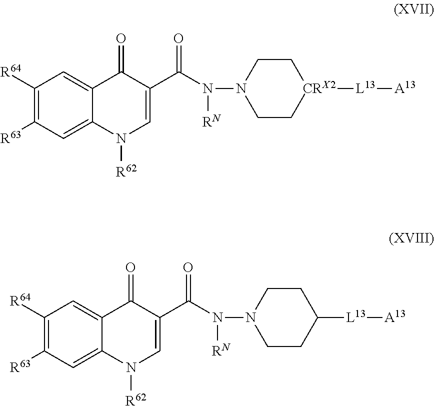

In some embodiments of the compound of formula (XIII), or the salt thereof, the compound of formula (XI), or the salt thereof, is a compound of formula (XIX):

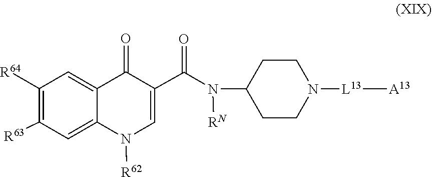

or a salt thereof,
wherein R⁶², R⁶³, R⁶⁴, L¹³, and A¹¹ are as defined in compounds of formula (XI); provided that when R⁶² is methyl and L¹³ is @¹³—CH₂-#¹³, then A¹³ is then A¹³ is (A¹³-1), C₆-C₁₄ aryl optionally substituted with 1, 2, 3, 4, 5, 6, 7, 8, or 9 R^{A13} substituents, or 5-14 membered heteroaryl substituted with 1, 2, 3, 4, 5, 6, 7, 8, or 9 R^{A3} substituents.

In some embodiments of the compounds of formulae (III), (IV), (V), (VI), (VII), (VIII), (IX), (X), (XI), (XII), (XIII), (XIV), (XV), (XVI), (XVII), (XVIII), and (XIX), or the salts thereof, R⁶² is selected from the group consisting of halogen, NO₂, C₁-C₆ alkyl, C₂-C₆ alkenyl, C₂-C₆ alkynyl, —(C₁-C₆ alkylene)-(C₆-C₁₄ aryl optionally substituted with 1, 2, 3, 4, 5, 6, 7, 8, or 9 R^{A3} substituents), —(C₁-C₆ alkylene)-(5-14 membered heteroaryl optionally substituted with 1, 2, 3, 4, 5, 6, 7, 8, or 9 R^{A3} substituents), C₁-C₆ haloalkyl, —OH, —O(C₁-C₆ alkyl), —O(C₁-C₆ haloalkyl), —(C₁-C₆ alkylene)-OH, —(C₁-C₆ alkylene)-O—(C₁-C₆ alkyl), —(C₁-C₆ alkylene)-O—(C₁-C₆ haloalkyl), —SH, —S(C₁-C₆ alkyl), —S(C₁-C₆ haloalkyl), —NH₂, —NH(C₁-C₆ alkyl), —NH(C₁-C₆ haloalkyl), —N(C₁-C₆ alkyl)₂, —N(C₁-C₆ haloalkyl)₂, —CN, —C(O)OH, —C(O)O(C₁-C₆ alkyl), —C(O)O(C₁-C₆ haloalkyl), —C(O)NH₂, —C(O)NH(C₁-C₆ alkyl), —C(O)NH(C₁-C₆ haloalkyl), —C(O)N(C₁-C₆ alkyl)₂, —C(O)N(C₁-C₆ haloalkyl)₂, —C(O)NR^{62-a}R^{62-b}, —S(O)₂OH, —S(O)₂O(C₁-C₆ alkyl), —S(O)₂O(C₁-C₆ haloalkyl), —S(O)₂NH₂, —S(O)₂NH(C₁-C₆ alkyl), —S(O)₂NH(C₁-C₆ haloalkyl), —S(O)₂N(C₁-C₆ alkyl)₂, —S(O)₂N(C₁-C₆ haloalkyl)₂, —S(O)₂NR^{62-a}R^{62-b}, —OC(O)H, —OC(O)(C₁-C₆ alkyl), —OC(O)(C₁-C₆ haloalkyl), —N(H)C(O)H, —N(H)C(O)(C₁-C₆ alkyl), —N(H)C(O)(C₁-C₆ haloalkyl), —N(C₁-C₆ alkyl)C(O)H, —N(C₁-C₆ alkyl)C(O)(C₁-C₆ alkyl), —N(C₁-C₆ alkyl)C(O)(C₁-C₆ haloalkyl), —N(C₁-C₆ haloalkyl)C(O)H, —N(C₁-C₆ haloalkyl)C(O)(C₁-C₆ alkyl), —N(C₁-C₆ haloalkyl)C(O)(C₁-C₆ haloalkyl), —OS(O)₂(C₁-C₆ alkyl), —OS(O)₂(C₁-C₆ haloalkyl), —N(H)S(O)₂(C₁-C₆ alkyl), —N(H)S(O)₂(C₁-C₆ haloalkyl), —N(C₁-C₆ alkyl)S(O)₂(C₁-C₆ alkyl), —N(C₁-C₆ alkyl)S(O)₂(C₁-C₆ haloalkyl), —N(C₁-C₆ haloalkyl)S(O)₂(C₁-C₆ alkyl), and —N(C₁-C₆ haloalkyl)S(O)₂(C₁-C₆ haloalkyl). In some embodiments, R⁶² is selected from the group consisting of halogen, C₁-C₆ alkyl, C₂-C₆ alkenyl, C₁-C₆ haloalkyl, —(C₁-C₆ alkylene)-O—(C₁-C₆ haloalkyl), and —CN. In some embodiments, R⁶² is halogen. In some embodiments, R⁶² is selected from the group consisting of fluoro, chloro, bromo, and iodo. In some embodiments, R⁶² is fluoro. In some embodiments, R⁶² is C₁-C₆ alkyl. In some embodiments, R⁶² is selected from methyl, ethyl, propyl, butyl, pentyl, and hexyl. In some embodiments, R⁶² is methyl. In some embodiments, R⁶² is propyl. In some embodiments, R⁶² is prop-1-yl. In some embodiments, R⁶² is prop-2-yl. In some embodiments, R⁶² is butyl. In some embodiments, R⁶² is n-butyl. In some embodiments, R⁶² is sec-butyl. In some embodiments, R⁶² is tert-butyl. In some embodiments, R⁶² is C₂-C₆ alkenyl. In some embodiments, R⁶² is selected from the group consisting of vinyl, propenyl, and butenyl. In some embodiments, R⁶² is vinyl. In some embodiments, R⁶² is C₁-C₆ haloalkyl. In some embodiments, R⁶² is selected from the group consisting of fluoromethyl, difluoromethyl, trifluoromethyl, 2,2,2-trifluoroethyl, and pentafluoroethyl. In some embodiments, R⁶² is trifluoromethyl. In some embodiments, R⁶² is —(C₁-C₆ alkylene)-O—(C₁-C₆ haloalkyl). In some embodiments, R⁶² is —CH₂—O—CF₃. In some embodiments, R⁶² is —CN.

In some embodiments of the compounds of formulae (III), (IV), (V), (VI), (VII), (VIII), (IX), (X), (XI), (XII), (XIII), (XIV), (XV), (XVI), (XVII), (XVIII), and (XIX), or the salts thereof, L¹³ is selected from the group consisting of

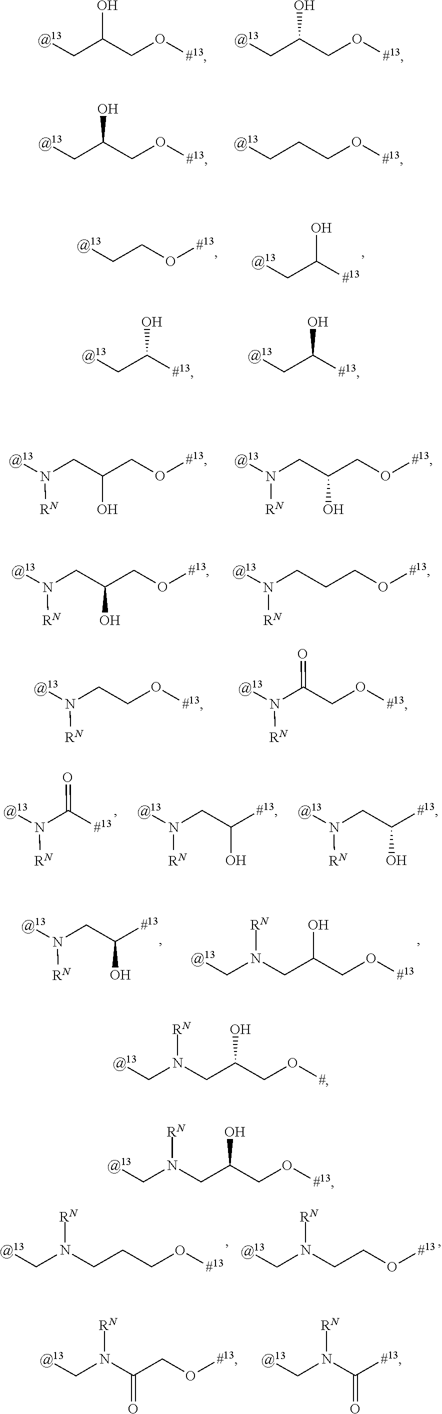

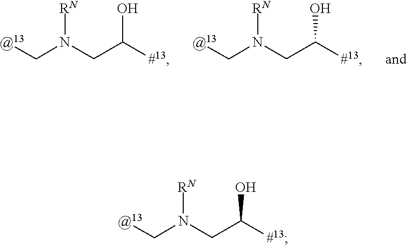

wherein #¹³ represents the attachment point to A¹³ and @¹³ represents the attachment point to the remainder of the molecule.

In some embodiments of the compounds of formulae (III), (VII), (VIII), (IX), (X), (XI), (XV), (XVI), (XVII), and (XVIII), or the salts thereof, L¹³ is selected from the group consisting of

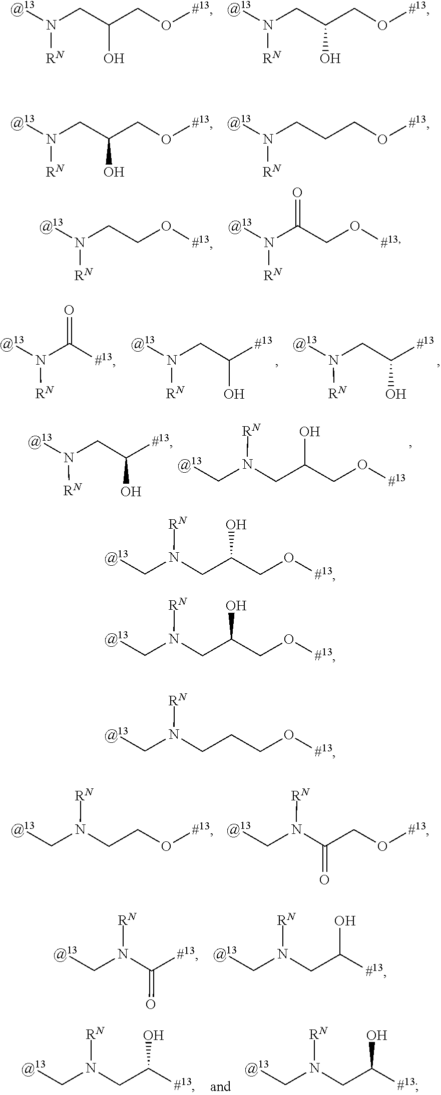

wherein #¹³ represents the attachment point to A¹³ and @¹³ represents the attachment point to the remainder of the molecule.

In some embodiments of the compounds of formulae (III), (IV), (V), (VI), (XI), (XII), (XIII), (XIV), and (XIX), or the salts thereof, L¹³ is selected from the group consisting of

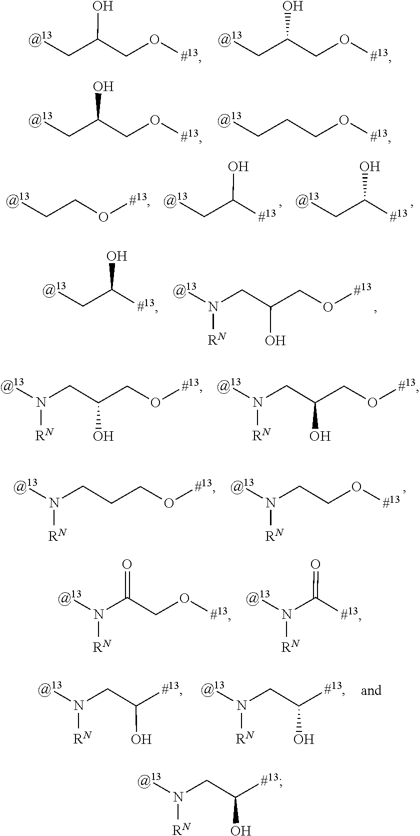

wherein #¹³ represents the attachment point to A¹³ and @¹³ represents the attachment point to the remainder of the molecule.

In some embodiments of the compounds of (III), (IV), (V), (VI), (VII), (VIII), (IX), (X), (XI), (XII), (XIII), (XIV), (XV), (XVI), (XVII), (XVIII), and (XIX), or the salts thereof, L¹³ is

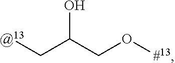

wherein #¹³ represents the attachment point to A¹³ and @¹³ represents the attachment point to the remainder of the molecule.

In some embodiments of the compounds of formulae (III), (IV), (V), (VI), (XI), (XII), (XIII), (XIV), and (XIX), or the salts thereof, L¹³ is

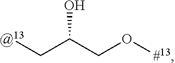

wherein #¹³ represents the attachment point to A¹³ and @¹³ represents the attachment point to the remainder of the molecule.

In some embodiments of the compounds of formulae (III), (IV), (V), (VI), (XI), (XII), (XIII), (XIV), and (XIX), or the pharmaceutically acceptable salts thereof, L¹³ is

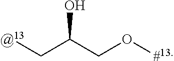

wherein #¹³ represents the attachment point to A¹³ and @¹³ represents the attachment point to the remainder of the molecule.

In some embodiments of the compounds of formulae (III), (IV), (V), (VI), (XI), (XII), (XIII), (XIV), and (XIX), or the salts thereof, R⁶³ and R⁶⁴ are each halogen. In some embodiments, R⁶³ is selected from the group consisting of fluoro, chloro, bromo, and iodo. In some embodiments, R⁶³ is fluoro. In some embodiments, R⁶³ is chloro. In some embodiments, R⁶³ is bromo. In some embodiments, R⁶³ is iodo. In some embodiments, R⁶ is selected from the group consisting of fluoro, chloro, bromo, and iodo. In some embodiments, R⁶ is fluoro. In some embodiments, R⁶ is chloro. In some embodiments, R⁶ is bromo. In some embodiments, R⁶ is iodo. In some embodiments, R⁶³ and R⁶, independently of each other, are selected from the group consisting of fluoro, chloro, bromo, and iodo. In some embodiments, R⁶³ is chloro and R⁶⁴ is fluoro.

In some embodiments of the compounds of formulae (III), (IV), (V), (VI), (XI), (XII), (XIII), (XIV), and (XIX), or the salts thereof, A¹³ is a substituent of formula (A¹³-1)

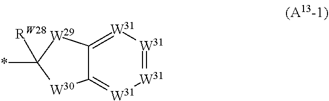

W²⁹ is selected from the group consisting of —C(R^{W29-1}R^{W29-2}), N(R^{W29-2})—, —C(R^{W29-1}R^{W29-1})N(R^{W29-2})—, —N(R^{W29-1})C(R^{W29-1}R^{W29-2})—, —C(R^{W29-1})═N—, —N═C(R^{W29-1})—, —O—, —C(R^{W29-1}R^{W29-1})O—, —OC(R^{W29-1}R^{W29-2})—, —S—, —C(R^{W29-1}R^{W29-1})S—, —SC(R^{W29-1}R^{W29-2})—, —C(R^{W29-1}R^{W29-1})C(R^{W29-1}R^{W29-2})—, and —CR^{W29-1}═CR^{W29-1}—,

wherein R^{W29-1} is H or R^{A1}, and R^{W29-2} is H or R^{A13}.

W³⁰ is selected from the group consisting of —C(R^{W30-1}R^{W30-2}), N(R^{W30-2}—, —C(R^{W30-1}R^{W30-1})N(R^{W30-2})—, —N(R^{W30-1})C(R^{W30-1}R^{W30-2})—, —C(R^{W30-1})═N—, —N═C(R^{W30-1})—, —O—, —C(R^{W30-1}R^{W30-1})O—, —OC(R^{W30-1}R^{W30-2})—, —S—, —C(R^{W30-1}R^{W30-1})S—, —SC(R^{W30-1}R^{W30-2})—, —C(R^{W30-1}R^{W30-1})C(R^{W30-1}R^{W30-2})—, and —CR^{W30-1}═CR^{W30-1}—,

wherein R^{W30-1} is H or R^{A13}, and R^{W30-2} is H or R^{A13}.

W³¹, independently at each occurrence, is CR^{W31} or N, wherein R^{W31} is H or R^{A13}.
R^{W28} is hydrogen or R^{A13}, or R^{W28} and R^{W29-2} are taken together to form a double bond between the carbon atom bearing R^{W28} and the atom bearing R^{W29-2}, or R^{W28} and R^{W30-2} are taken together to form a double bond between the carbon atom bearing R^{W28} and the atom bearing R^{W30-2}. In some embodiments, (A¹³-1) is selected from the group consisting of

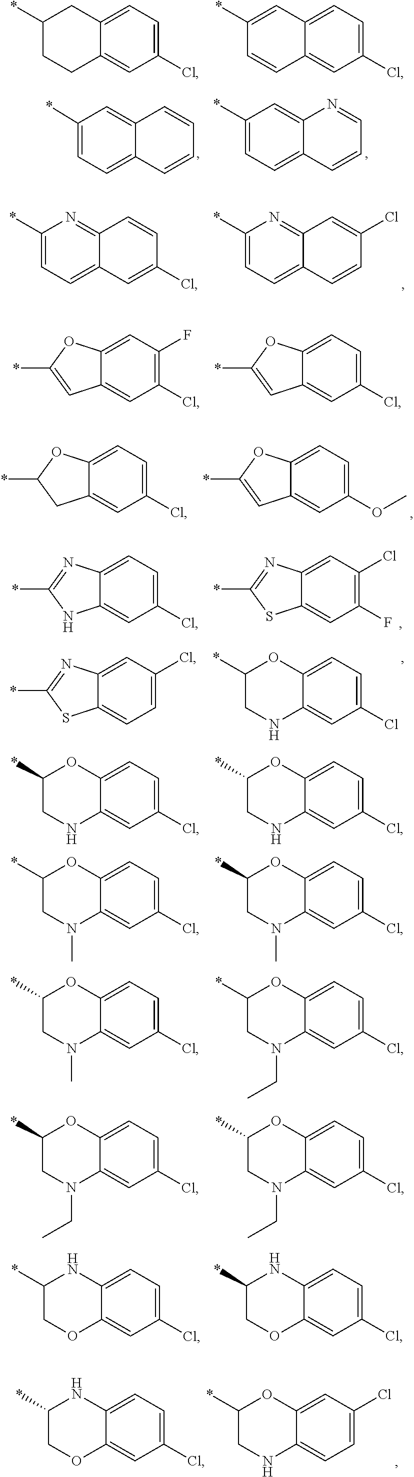

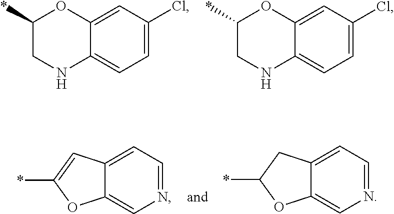

In some embodiments, (A¹³-1) is

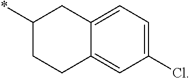 [SMILES: *C1CCc2cc(Cl)ccc2C1]

In some embodiments, (A¹³-1) is selected from the group consisting of

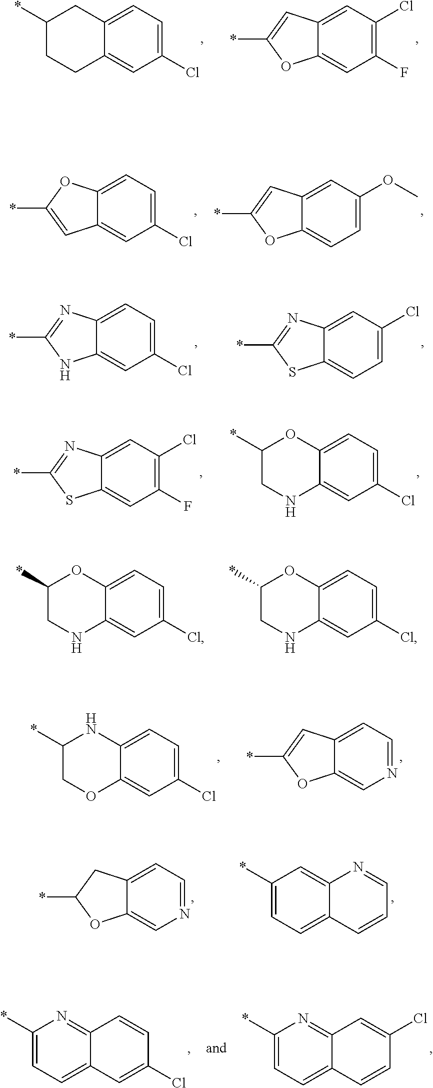

In some embodiments, (A¹³-1) is

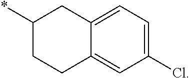 [SMILES: *C1CCc2cc(Cl)ccc2C1]

In some embodiments, (A¹³-1) is

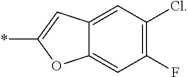 [SMILES: *c1cc2cc(Cl)c(F)cc2o1]

In some embodiments, (A¹³-1) is

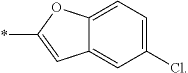 [SMILES: *c1cc2cc(Cl)ccc2o1]

In some embodiments, (A¹³-1) is

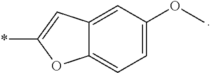 [SMILES: *c1cc2cc(OC)ccc2o1]

In some embodiments, (A¹³-1) is

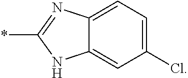 [SMILES: *c1nc2ccc(Cl)cc2[nH]1]

In some embodiments, (A¹³-1) is

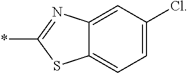 [SMILES: *c1nc2cc(Cl)ccc2s1]

In some embodiments, (A¹³-1) is

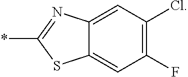 [SMILES: *c1nc2cc(Cl)c(F)cc2s1]

In some embodiments, (A¹³-1) is

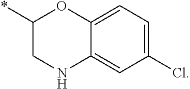 [SMILES: *C1CNc2cc(Cl)ccc2O1]

In some embodiments, (A¹³-1) is

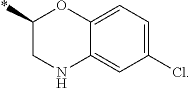 [SMILES: *[C@@H]1CNc2cc(Cl)ccc2O1]

In some embodiments, (A¹³-1) is

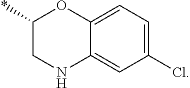 [SMILES: *[C@H]1CNc2cc(Cl)ccc2O1]

In some embodiments, (A¹³-1) is

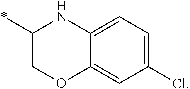 [SMILES: *C1COc2cc(Cl)ccc2N1]

In some embodiments, (A¹³-1) is

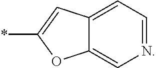 [SMILES: *c1cc2ccncc2o1]

In some embodiments, (A¹³-1) is

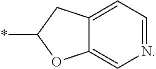 [SMILES: *C1Cc2ccncc2O1]

In some embodiments, (A¹³-1) is

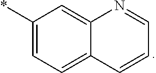 [SMILES: *c1ccc2cccnc2c1]

In some embodiments, (A¹³-1) is

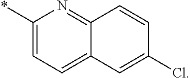 [SMILES: *c1ccc2cc(Cl)ccc2n1]

In some embodiments, (A¹³-1) is

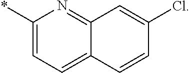 [SMILES: *c1ccc2ccc(Cl)cc2n1]

In some embodiments of the compounds of formulae (III), (IV), (V), (VI), (XI), (XII), (XIII), (XIV), and (XIX), or the salts thereof, A¹³ is C₆-C₁₄ aryl optionally substituted with 1, 2, 3, 4, 5, 6, 7, 8, or 9 R^{A3} substituents. In some embodiments, A¹³ is phenyl optionally substituted with 1, 2, 3, 4, or 5 R^{A3} substituents. In some embodiments, A¹³ is phenyl substituted with two R^{A3} substituents. In some embodiments, A¹³ is phenyl substituted with two R^{A3} substituents and R^{A13}, independently at each occurrence, is halogen. In embodiments, A¹³ is phenyl substituted with two R^{A13} substituents and R^{A}, independently at each occurrence, is selected from the group consisting of fluoro, chloro, bromo, and iodo. In some embodiments, A¹³ is phenyl substituted with two R^{A13} substituents and one R^{A13} is fluoro and the other R^{A13} is chloro. In some embodiments, A¹³ is 1-chloro-2-fluoro-benz-4-yl. In some embodiments, A¹³ is selected from the group consisting of

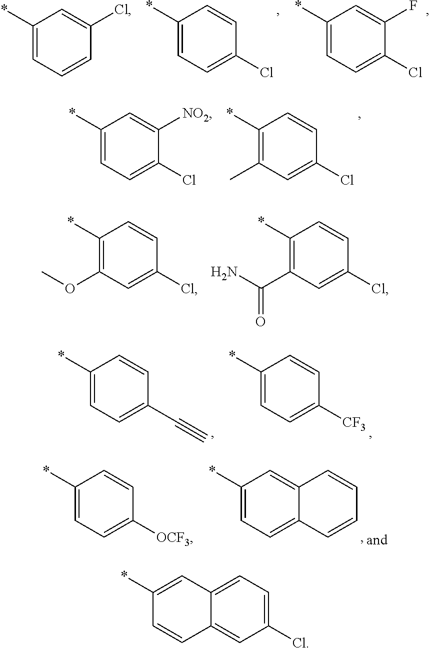

In some embodiments, A¹³ is selected from the group consisting of

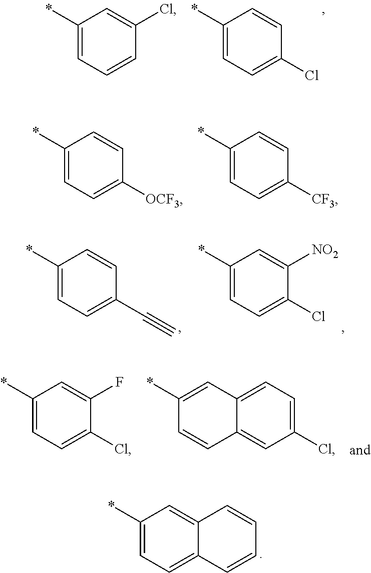

In some embodiments, A¹³ is

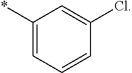 [SMILES: *c1cccc(Cl)c1]

In some embodiments, A¹³ is

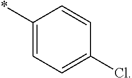 [SMILES: *c1ccc(Cl)cc1]

In some embodiments, A¹³ is

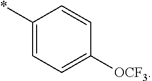 [SMILES: *c1ccc(OC(F)(F)F)cc1]

In some embodiments, A¹³ is

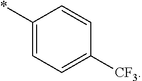 [SMILES: *c1ccc(C(F)(F)F)cc1]

In some embodiments, A¹³ is

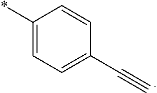 [SMILES: *c1ccc(C#C)cc1]

In some embodiments, A¹³is

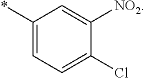 [SMILES: *c1ccc(Cl)c([N+](=O)[O-])c1]

In some embodiments, A¹³is

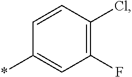 [SMILES: *c1ccc(Cl)c(F)c1]

wherein * represents the attachment point to the remainder of the molecule. In some embodiments, A¹³ is

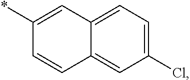 [SMILES: *c1ccc2cc(Cl)ccc2c1]

wherein * represents the attachment point to the remainder of the molecule. In some embodiments, A¹³ is

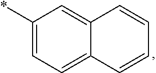 [SMILES: *c1ccc2ccccc2c1]

wherein * represents the attachment point to the remainder of the molecule.

In some embodiments of the compounds of formulae (III), (IV), (V), (VI), (XI), (XII), (XIII), (XIV), and (XIX), or the salts thereof, 5-14 membered heteroaryl optionally substituted with 1, 2, 3, 4, 5, 6, 7, 8, or 9 R^{A13} substituents. In some embodiments, A¹³ is pyridyl optionally substituted with 1, 2, 3, 4, or 5 R^{A13} substituents. In some embodiments, A¹³ is pyrazinyl optionally substituted with 1, 2, 3, 4, or 5 R^{A13} substituents. In some embodiments, A¹³ is quinolinyl optionally substituted with 1, 2, 3, 4, or 5 R^{A} substituents. In some embodiments, A¹³ is selected from the group consisting of

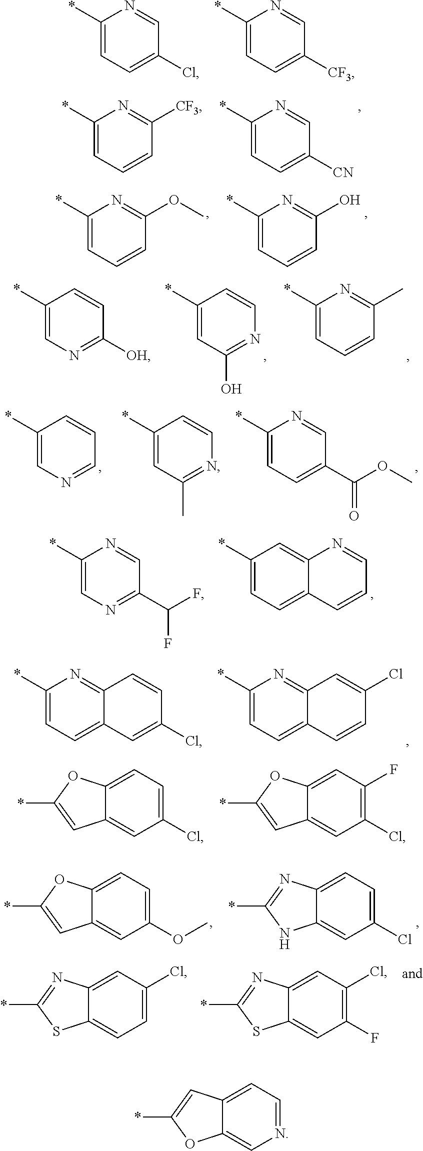

In some embodiments, A¹³ is selected from the group consisting of

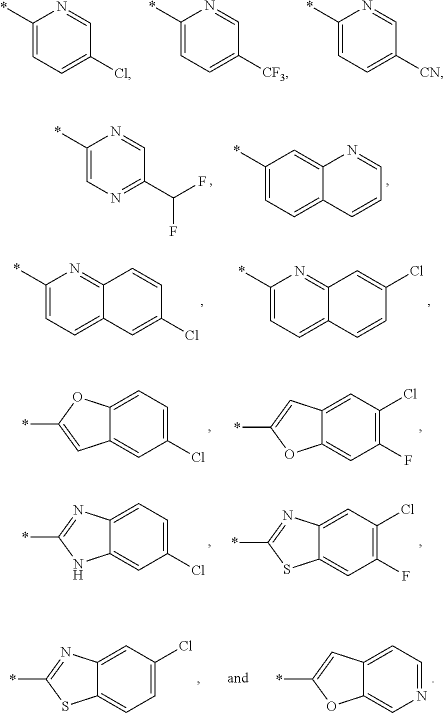

In some embodiments, A¹³ is

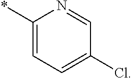 [SMILES: *c1ccc(Cl)cn1]

In some embodiments, A¹³ is

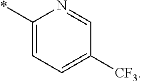 [SMILES: *c1ccc(C(F)(F)F)cn1]

In some embodiments, A¹³ is

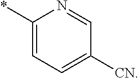 [SMILES: *c1ccc(C#N)cn1]

In some embodiments, A¹³ is

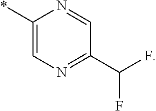 [SMILES: *c1cnc(C(F)F)cn1]

In some embodiments, A¹³ is

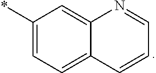 [SMILES: *c1ccc2cccnc2c1]

In some embodiments, A¹³ is

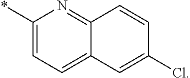 [SMILES: *c1ccc2cc(Cl)ccc2n1]

In some embodiments, A¹³ is

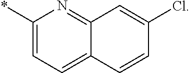 [SMILES: *c1ccc2ccc(Cl)cc2n1]

In some embodiments, A¹³ is

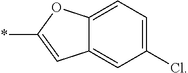 [SMILES: *c1cc2cc(Cl)ccc2o1]

In some embodiments, A¹³ is

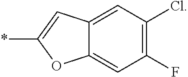 [SMILES: *c1cc2cc(Cl)c(F)cc2o1]

In some embodiments, A¹³ is

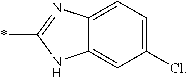 [SMILES: *c1nc2ccc(Cl)cc2[nH]1]

In some embodiments, A¹³ is

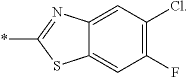 [SMILES: *c1nc2cc(Cl)c(F)cc2s1]

In some embodiments, A¹³ is

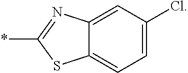 [SMILES: *c1nc2cc(Cl)ccc2s1]

In some embodiments, A¹³ is

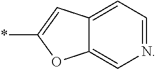 [SMILES: *c1cc2ccncc2o1]

In the descriptions herein, it is understood that every description, variation, embodiment or aspect of a moiety may be combined with every description, variation, embodiment or aspect of other moieties the same as if each and every combination of descriptions is specifically and individually listed. For example, every description, variation, embodiment or aspect provided herein with respect to A¹ of formula (A-1) may be combined with every description, variation, embodiment or aspect of A², R¹, R², R³, R⁴, R⁵, R⁶, R⁷, and R⁸ the same as if each and every combination were specifically and individually listed. It is also understood that all descriptions, variations, embodiments or aspects of formula (I), where applicable, apply equally to other formulae detailed herein, and are equally described, the same as if each and every description, variation, embodiment or aspect were separately and individually listed for all formulae.

Also provided are salts of compounds referred to herein, such as pharmaceutically acceptable salts. The present disclosure also includes any or all of the stereochemical forms, including any enantiomeric or diastereomeric forms, and any tautomers or other forms of the compounds described. Thus, if a particular stereochemical form, such as a specific enantiomeric form or diastereomeric form, is depicted for a given compound, then it is understood that any or all stereochemical forms, including any enantiomeric or diastereomeric forms, and any tautomers or other forms of any of that same compound are herein described and embraced by the invention.

A compound as detailed herein may in one aspect be in a purified form and compositions comprising a compound in purified forms are detailed herein. Compositions comprising a compound as detailed herein or a salt thereof are provided, such as compositions of substantially pure compounds. In some embodiments, a composition containing a compound as detailed herein or a salt thereof is in substantially pure form. Unless otherwise stated, “substantially pure” intends a composition that contains no more than 35% impurity, wherein the impurity denotes a compound other than the compound comprising the majority of the composition or a salt thereof. In some embodiments, a composition of substantially pure compound or a salt thereof is provided wherein the composition contains no more than 25%, 20%, 15%, 10%, or 5% impurity. In some embodiments, a composition of substantially pure compound or a salt thereof is provided wherein the composition contains or no more than 3%, 2%, 1% or 0.5% impurity.

In some embodiments, provided is compound selected from compounds in Table 1, or a stereoisomer, tautomer, solvate, prodrug or salt thereof. Although certain compounds described in Table 1 are presented as specific stereoisomers and/or in a non-stereochemical form, it is understood that any or all stereochemical forms, including any enantiomeric or diastereomeric forms, and any tautomers or other forms of any of the compounds of Table 1 are herein described.

TABLE 1

| Cpd No. | Structure |
|---|---|
| 1 | 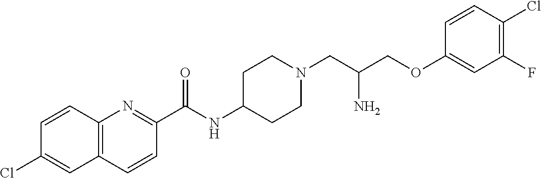 [SMILES: NC(COc1ccc(Cl)c(F)c1)CN1CCC(NC(=O)c2ccc3cc(Cl)ccc3n2)CC1] |
| 2 | 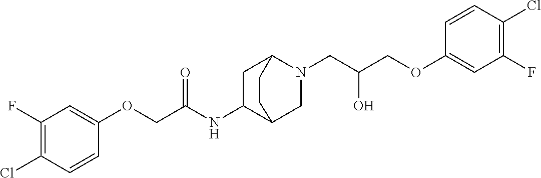 [SMILES: O=C(COc1ccc(Cl)c(F)c1)NC1CC2CCC1CN2CC(O)COc1ccc(Cl)c(F)c1] |
| 3 | 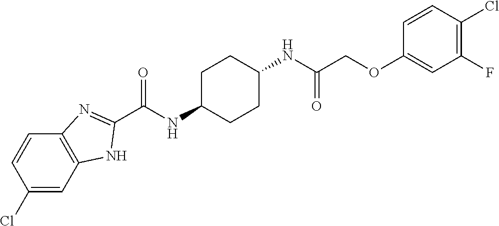 [SMILES: O=C(COc1ccc(Cl)c(F)c1)N[C@H]1CC[C@H](NC(=O)c2nc3ccc(Cl)cc3[nH]2)CC1] |
| 4 | 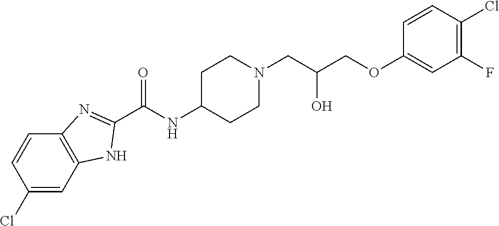 [SMILES: O=C(NC1CCN(CC(O)COc2ccc(Cl)c(F)c2)CC1)c1nc2ccc(Cl)cc2[nH]1] |
| 5 | 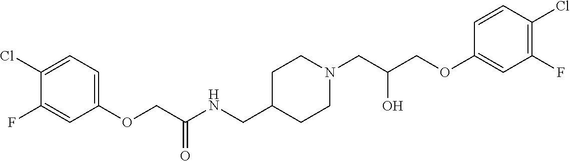 [SMILES: O=C(COc1ccc(Cl)c(F)c1)NCC1CCN(CC(O)COc2ccc(Cl)c(F)c2)CC1] |
| 6 | 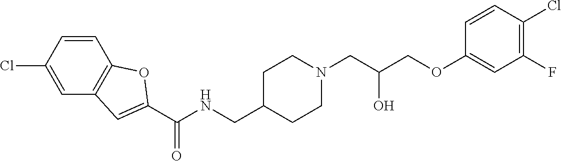 [SMILES: O=C(NCC1CCN(CC(O)COc2ccc(Cl)c(F)c2)CC1)c1cc2cc(Cl)ccc2o1] |
| 7 | 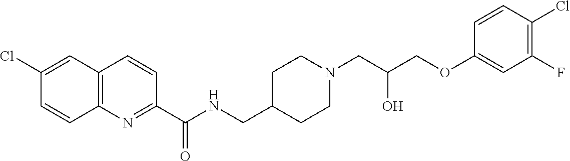 [SMILES: O=C(NCC1CCN(CC(O)COc2ccc(Cl)c(F)c2)CC1)c1ccc2cc(Cl)ccc2n1] |
| 8 | 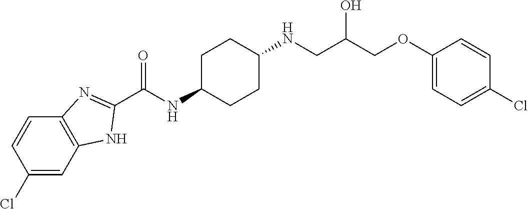 [SMILES: O=C(N[C@H]1CC[C@H](NCC(O)COc2ccc(Cl)cc2)CC1)c1nc2ccc(Cl)cc2[nH]1] |
| 9 | 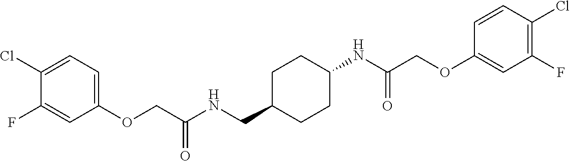 [SMILES: O=C(COc1ccc(Cl)c(F)c1)NC[C@H]1CC[C@H](NC(=O)COc2ccc(Cl)c(F)c2)CC1] |
| 10 | 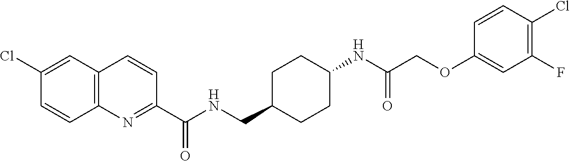 [SMILES: O=C(COc1ccc(Cl)c(F)c1)N[C@H]1CC[C@H](CNC(=O)c2ccc3cc(Cl)ccc3n2)CC1] |
| 11 | 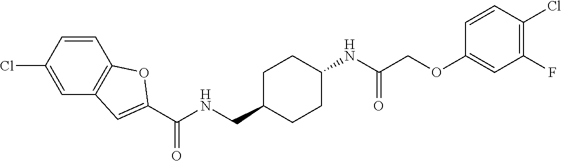 [SMILES: O=C(COc1ccc(Cl)c(F)c1)N[C@H]1CC[C@H](CNC(=O)c2cc3cc(Cl)ccc3o2)CC1] |
| 12 | 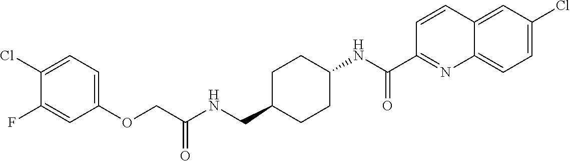 [SMILES: O=C(COc1ccc(Cl)c(F)c1)NC[C@H]1CC[C@H](NC(=O)c2ccc3cc(Cl)ccc3n2)CC1] |
| 13 | 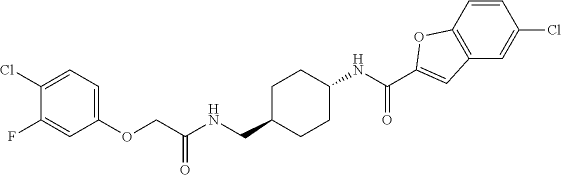 [SMILES: O=C(COc1ccc(Cl)c(F)c1)NC[C@H]1CC[C@H](NC(=O)c2cc3cc(Cl)ccc3o2)CC1] |
| 14 | 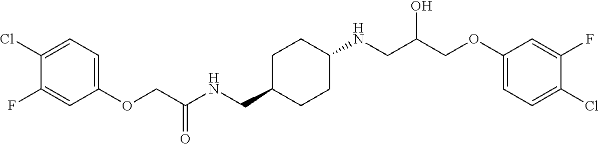 [SMILES: O=C(COc1ccc(Cl)c(F)c1)NC[C@H]1CC[C@H](NCC(O)COc2ccc(Cl)c(F)c2)CC1] |
| 15 | 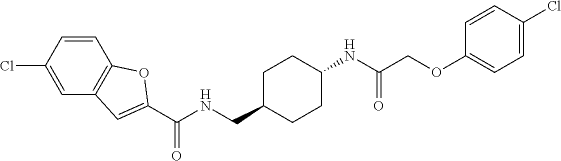 [SMILES: O=C(COc1ccc(Cl)cc1)N[C@H]1CC[C@H](CNC(=O)c2cc3cc(Cl)ccc3o2)CC1] |
| 16 | 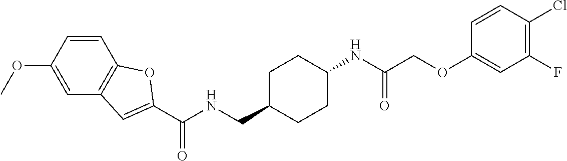 [SMILES: COc1ccc2oc(C(=O)NC[C@H]3CC[C@H](NC(=O)COc4ccc(Cl)c(F)c4)CC3)cc2c1] |
| 17 | 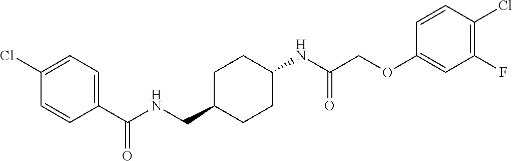 [SMILES: O=C(COc1ccc(Cl)c(F)c1)N[C@H]1CC[C@H](CNC(=O)c2ccc(Cl)cc2)CC1] |
| 18 | 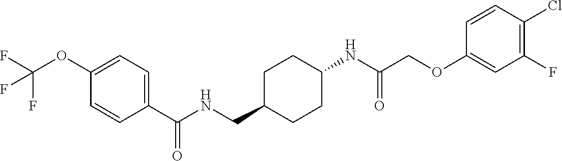 [SMILES: O=C(COc1ccc(Cl)c(F)c1)N[C@H]1CC[C@H](CNC(=O)c2ccc(OC(F)(F)F)cc2)CC1] |
| 19 | 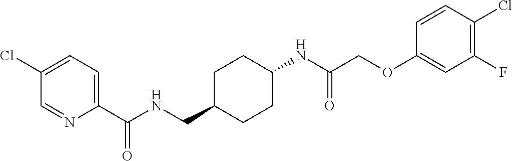 [SMILES: O=C(COc1ccc(Cl)c(F)c1)N[C@H]1CC[C@H](CNC(=O)c2ccc(Cl)cn2)CC1] |
| 20 | 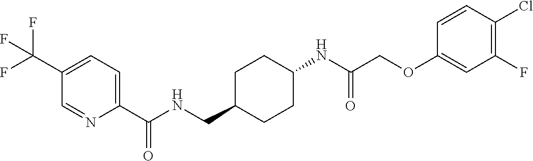 [SMILES: O=C(COc1ccc(Cl)c(F)c1)N[C@H]1CC[C@H](CNC(=O)c2ccc(C(F)(F)F)cn2)CC1] |
| 21 | 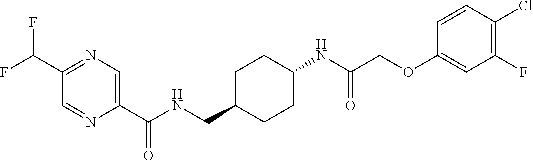 [SMILES: O=C(COc1ccc(Cl)c(F)c1)N[C@H]1CC[C@H](CNC(=O)c2cnc(C(F)F)cn2)CC1] |
| 22 | 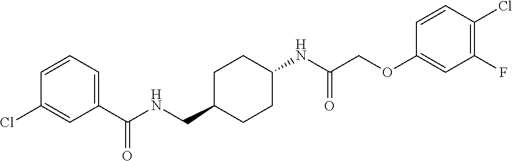 [SMILES: O=C(COc1ccc(Cl)c(F)c1)N[C@H]1CC[C@H](CNC(=O)c2cccc(Cl)c2)CC1] |
| 23 | 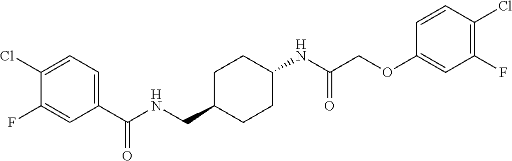 [SMILES: O=C(COc1ccc(Cl)c(F)c1)N[C@H]1CC[C@H](CNC(=O)c2ccc(Cl)c(F)c2)CC1] |
| 24 | 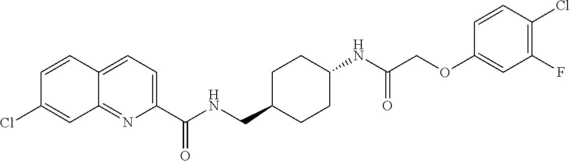 [SMILES: O=C(COc1ccc(Cl)c(F)c1)N[C@H]1CC[C@H](CNC(=O)c2ccc3ccc(Cl)cc3n2)CC1] |
| 25 | 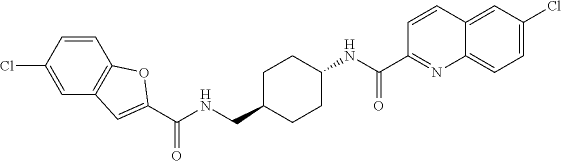 [SMILES: O=C(N[C@H]1CC[C@H](CNC(=O)c2cc3cc(Cl)ccc3o2)CC1)c1ccc2cc(Cl)ccc2n1] |
| 26 | 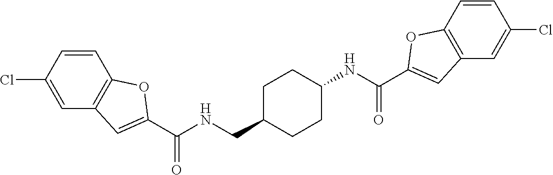 [SMILES: O=C(NC[C@H]1CC[C@H](NC(=O)c2cc3cc(Cl)ccc3o2)CC1)c1cc2cc(Cl)ccc2o1] |
| 27 | 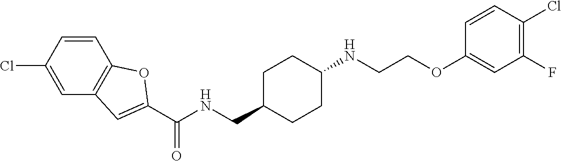 [SMILES: O=C(NC[C@H]1CC[C@H](NCCOc2ccc(Cl)c(F)c2)CC1)c1cc2cc(Cl)ccc2o1] |
| 28 | 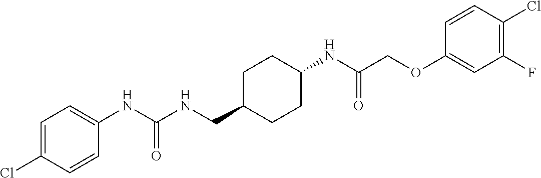 [SMILES: O=C(NC[C@H]1CC[C@H](NC(=O)COc2ccc(Cl)c(F)c2)CC1)Nc1ccc(Cl)cc1] |
| 29 | 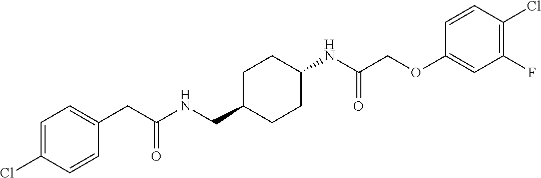 [SMILES: O=C(Cc1ccc(Cl)cc1)NC[C@H]1CC[C@H](NC(=O)COc2ccc(Cl)c(F)c2)CC1] |
| 30 | 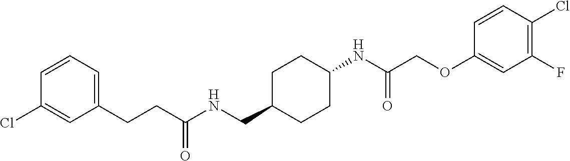 [SMILES: O=C(CCc1cccc(Cl)c1)NC[C@H]1CC[C@H](NC(=O)COc2ccc(Cl)c(F)c2)CC1] |
| 31 | 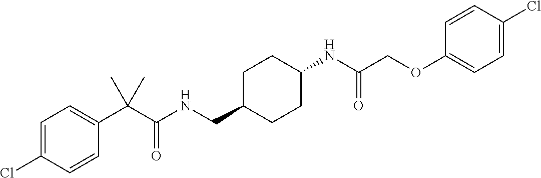 [SMILES: CC(C)(C(=O)NC[C@H]1CC[C@H](NC(=O)COc2ccc(Cl)cc2)CC1)c1ccc(Cl)cc1] |
| 32 | 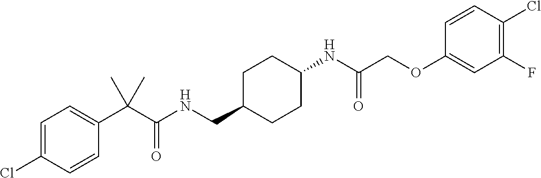 [SMILES: CC(C)(C(=O)NC[C@H]1CC[C@H](NC(=O)COc2ccc(Cl)c(F)c2)CC1)c1ccc(Cl)cc1] |
| 33 | 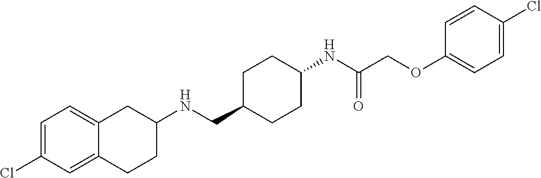 [SMILES: O=C(COc1ccc(Cl)cc1)N[C@H]1CC[C@H](CNC2CCc3cc(Cl)ccc3C2)CC1] |
| 34 | 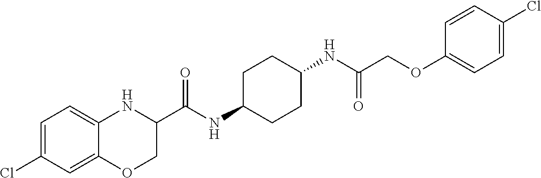 [SMILES: O=C(COc1ccc(Cl)cc1)N[C@H]1CC[C@H](NC(=O)C2COc3cc(Cl)ccc3N2)CC1] |
| 35 | 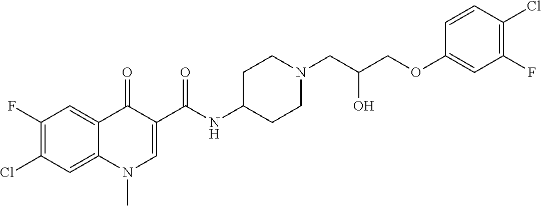 [SMILES: Cn1cc(C(=O)NC2CCN(CC(O)COc3ccc(Cl)c(F)c3)CC2)c(=O)c2cc(F)c(Cl)cc21] |
| 36 | 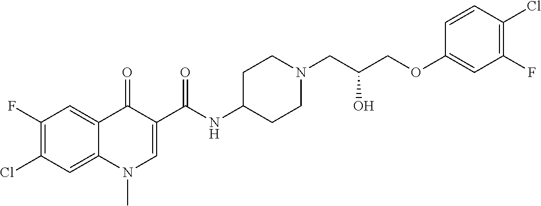 [SMILES: Cn1cc(C(=O)NC2CCN(C[C@@H](O)COc3ccc(Cl)c(F)c3)CC2)c(=O)c2cc(F)c(Cl)cc21] |
| 37 | 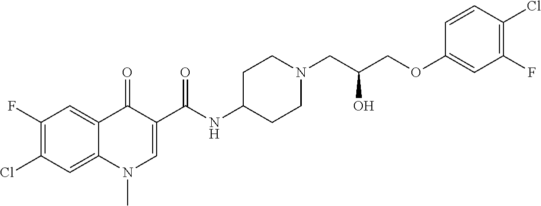 [SMILES: Cn1cc(C(=O)NC2CCN(C[C@H](O)COc3ccc(Cl)c(F)c3)CC2)c(=O)c2cc(F)c(Cl)cc21] |
| 38 | 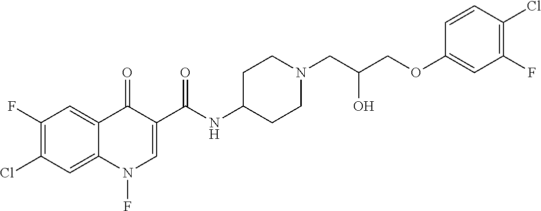 [SMILES: O=C(NC1CCN(CC(O)COc2ccc(Cl)c(F)c2)CC1)c1cn(F)c2cc(Cl)c(F)cc2c1=O] |
| 39 | 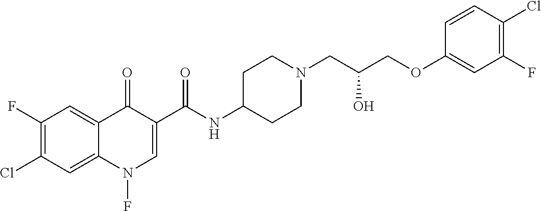 [SMILES: O=C(NC1CCN(C[C@@H](O)COc2ccc(Cl)c(F)c2)CC1)c1cn(F)c2cc(Cl)c(F)cc2c1=O] |
| 40 | 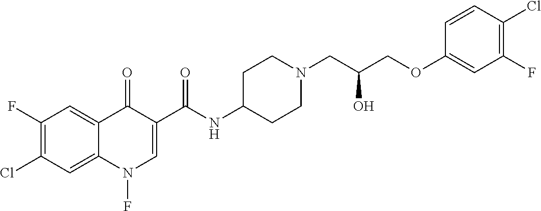 [SMILES: O=C(NC1CCN(C[C@H](O)COc2ccc(Cl)c(F)c2)CC1)c1cn(F)c2cc(Cl)c(F)cc2c1=O] |
| 41 | 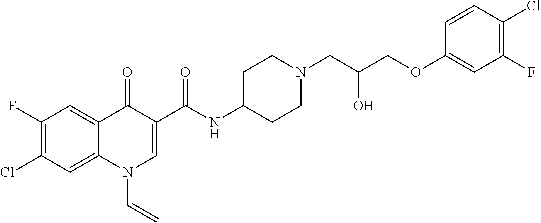 [SMILES: C=Cn1cc(C(=O)NC2CCN(CC(O)COc3ccc(Cl)c(F)c3)CC2)c(=O)c2cc(F)c(Cl)cc21] |
| 42 | 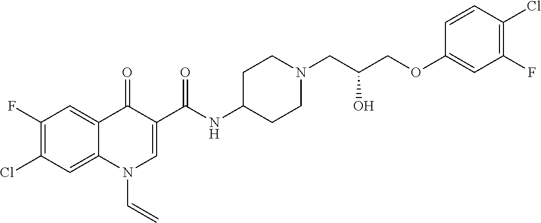 [SMILES: C=Cn1cc(C(=O)NC2CCN(C[C@@H](O)COc3ccc(Cl)c(F)c3)CC2)c(=O)c2cc(F)c(Cl)cc21] |
| 43 | 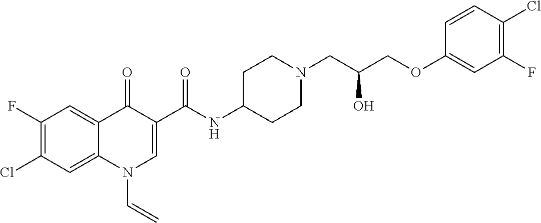 [SMILES: C=Cn1cc(C(=O)NC2CCN(C[C@H](O)COc3ccc(Cl)c(F)c3)CC2)c(=O)c2cc(F)c(Cl)cc21] |
| 44 | 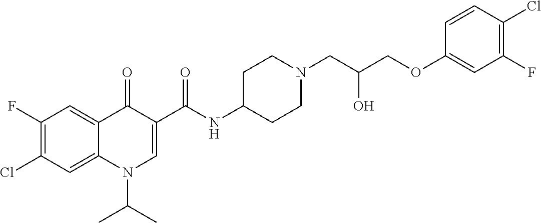 [SMILES: CC(C)n1cc(C(=O)NC2CCN(CC(O)COc3ccc(Cl)c(F)c3)CC2)c(=O)c2cc(F)c(Cl)cc21] |
| 45 | 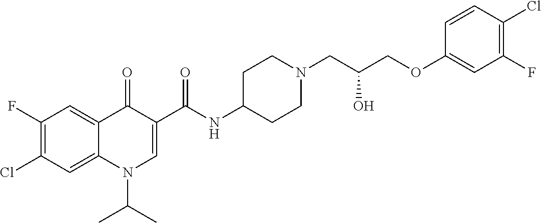 [SMILES: CC(C)n1cc(C(=O)NC2CCN(C[C@@H](O)COc3ccc(Cl)c(F)c3)CC2)c(=O)c2cc(F)c(Cl)cc21] |
| 46 | 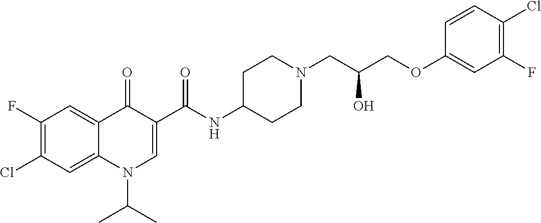 [SMILES: CC(C)n1cc(C(=O)NC2CCN(C[C@H](O)COc3ccc(Cl)c(F)c3)CC2)c(=O)c2cc(F)c(Cl)cc21] |
| 47 | 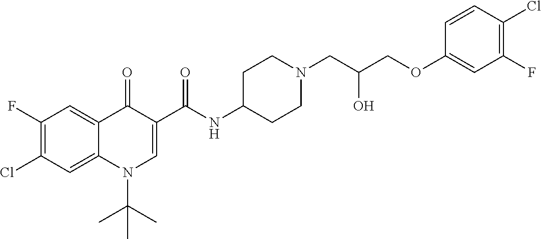 [SMILES: CC(C)(C)n1cc(C(=O)NC2CCN(CC(O)COc3ccc(Cl)c(F)c3)CC2)c(=O)c2cc(F)c(Cl)cc21] |
| 48 | 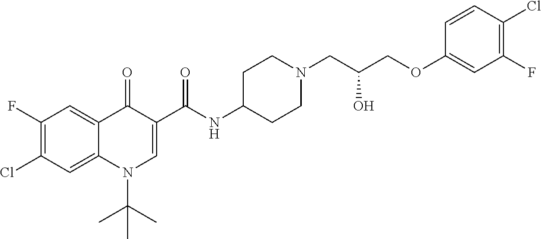 [SMILES: CC(C)(C)n1cc(C(=O)NC2CCN(C[C@@H](O)COc3ccc(Cl)c(F)c3)CC2)c(=O)c2cc(F)c(Cl)cc21] |
| 49 | 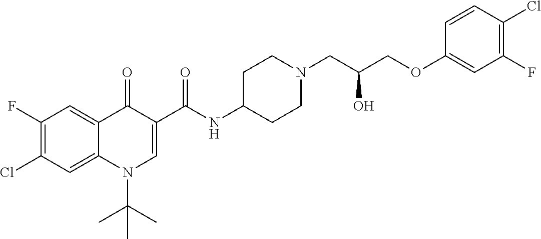 [SMILES: CC(C)(C)n1cc(C(=O)NC2CCN(C[C@H](O)COc3ccc(Cl)c(F)c3)CC2)c(=O)c2cc(F)c(Cl)cc21] |
| 50 | 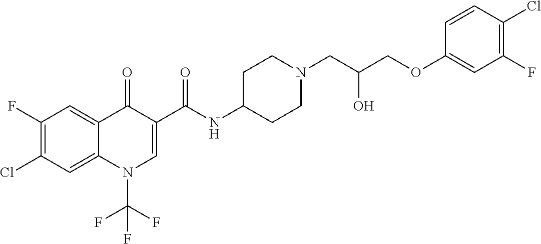 [SMILES: O=C(NC1CCN(CC(O)COc2ccc(Cl)c(F)c2)CC1)c1cn(C(F)(F)F)c2cc(Cl)c(F)cc2c1=O] |
| 51 | 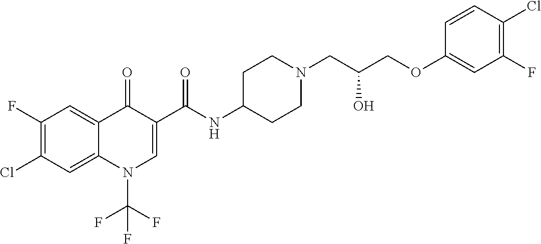 [SMILES: O=C(NC1CCN(C[C@@H](O)COc2ccc(Cl)c(F)c2)CC1)c1cn(C(F)(F)F)c2cc(Cl)c(F)cc2c1=O] |
| 52 | 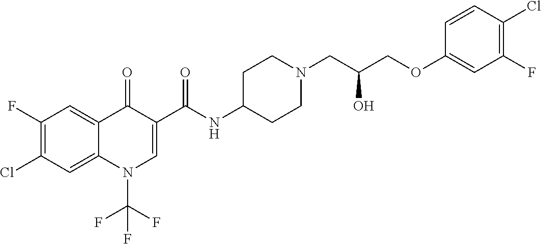 [SMILES: O=C(NC1CCN(C[C@H](O)COc2ccc(Cl)c(F)c2)CC1)c1cn(C(F)(F)F)c2cc(Cl)c(F)cc2c1=O] |
| 53 | 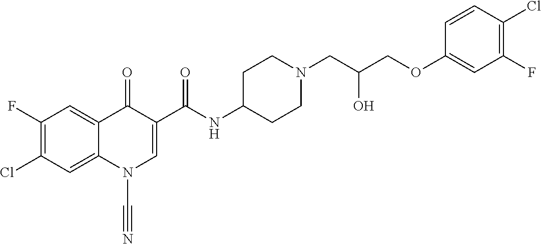 [SMILES: N#Cn1cc(C(=O)NC2CCN(CC(O)COc3ccc(Cl)c(F)c3)CC2)c(=O)c2cc(F)c(Cl)cc21] |
| 54 | 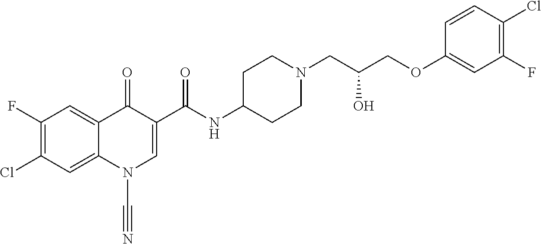 [SMILES: N#Cn1cc(C(=O)NC2CCN(C[C@@H](O)COc3ccc(Cl)c(F)c3)CC2)c(=O)c2cc(F)c(Cl)cc21] |
| 55 | 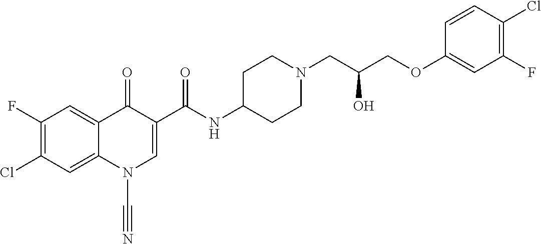 [SMILES: N#Cn1cc(C(=O)NC2CCN(C[C@H](O)COc3ccc(Cl)c(F)c3)CC2)c(=O)c2cc(F)c(Cl)cc21] |
| 56 | 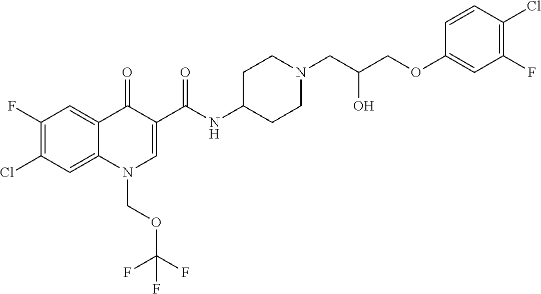 [SMILES: O=C(NC1CCN(CC(O)COc2ccc(Cl)c(F)c2)CC1)c1cn(COC(F)(F)F)c2cc(Cl)c(F)cc2c1=O] |
| 57 | 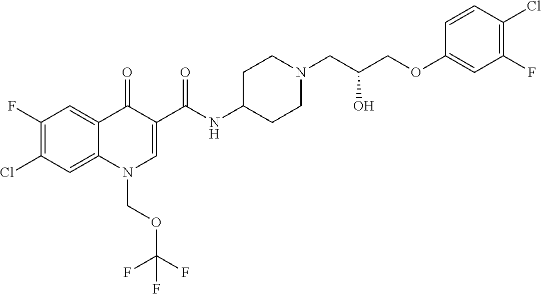 [SMILES: O=C(NC1CCN(C[C@@H](O)COc2ccc(Cl)c(F)c2)CC1)c1cn(COC(F)(F)F)c2cc(Cl)c(F)cc2c1=O] |
| 58 | 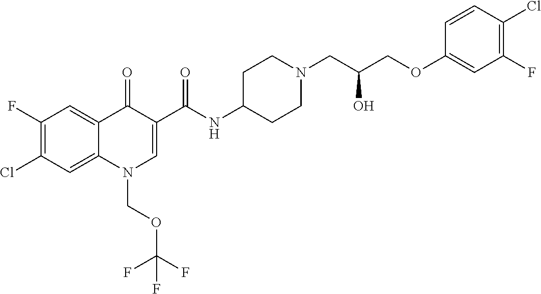 [SMILES: O=C(NC1CCN(C[C@H](O)COc2ccc(Cl)c(F)c2)CC1)c1cn(COC(F)(F)F)c2cc(Cl)c(F)cc2c1=O] |
| 59 | 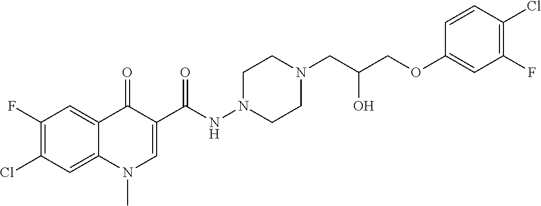 [SMILES: Cn1cc(C(=O)NN2CCN(CC(O)COc3ccc(Cl)c(F)c3)CC2)c(=O)c2cc(F)c(Cl)cc21] |
| 60 | 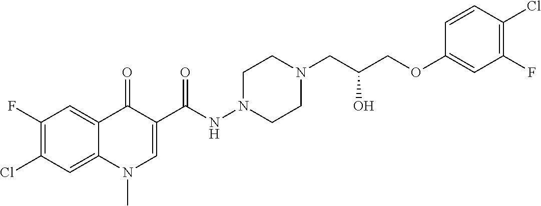 [SMILES: Cn1cc(C(=O)NN2CCN(C[C@@H](O)COc3ccc(Cl)c(F)c3)CC2)c(=O)c2cc(F)c(Cl)cc21] |
| 61 | 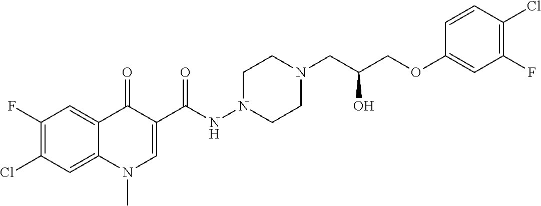 [SMILES: Cn1cc(C(=O)NN2CCN(C[C@H](O)COc3ccc(Cl)c(F)c3)CC2)c(=O)c2cc(F)c(Cl)cc21] |
| 62 | 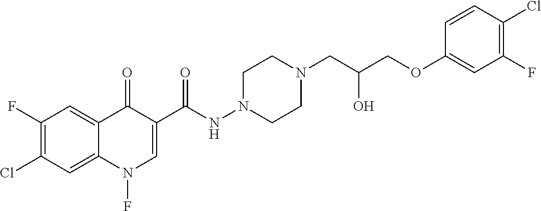 [SMILES: O=C(NN1CCN(CC(O)COc2ccc(Cl)c(F)c2)CC1)c1cn(F)c2cc(Cl)c(F)cc2c1=O] |
| 63 | 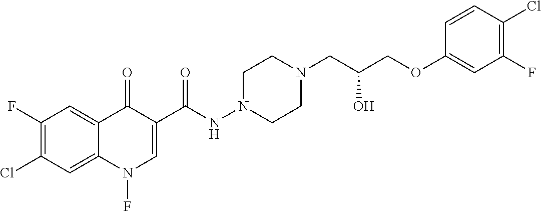 [SMILES: O=C(NN1CCN(C[C@@H](O)COc2ccc(Cl)c(F)c2)CC1)c1cn(F)c2cc(Cl)c(F)cc2c1=O] |
| 64 | 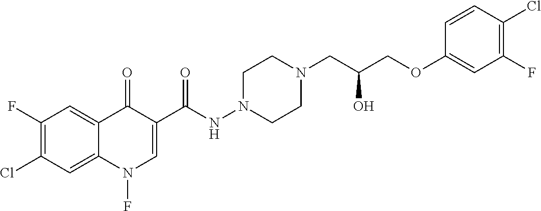 [SMILES: O=C(NN1CCN(C[C@H](O)COc2ccc(Cl)c(F)c2)CC1)c1cn(F)c2cc(Cl)c(F)cc2c1=O] |
| 65 | 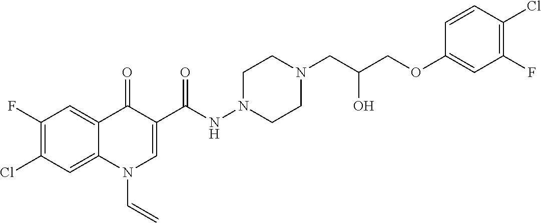 [SMILES: C=Cn1cc(C(=O)NN2CCN(CC(O)COc3ccc(Cl)c(F)c3)CC2)c(=O)c2cc(F)c(Cl)cc21] |
| 66 | 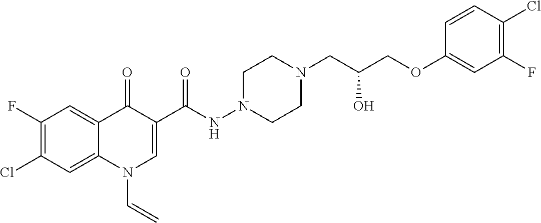 [SMILES: C=Cn1cc(C(=O)NN2CCN(C[C@@H](O)COc3ccc(Cl)c(F)c3)CC2)c(=O)c2cc(F)c(Cl)cc21] |
| 67 | 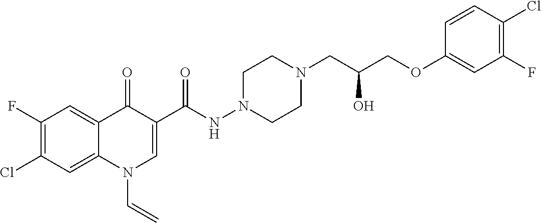 [SMILES: C=Cn1cc(C(=O)NN2CCN(C[C@H](O)COc3ccc(Cl)c(F)c3)CC2)c(=O)c2cc(F)c(Cl)cc21] |
| 68 | 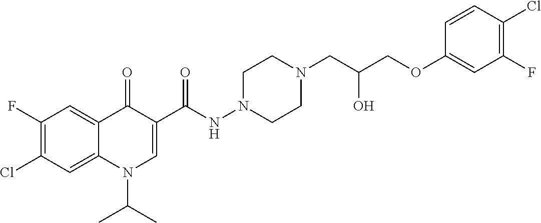 [SMILES: CC(C)n1cc(C(=O)NN2CCN(CC(O)COc3ccc(Cl)c(F)c3)CC2)c(=O)c2cc(F)c(Cl)cc21] |
| 69 | 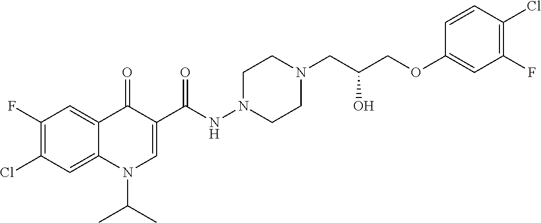 [SMILES: CC(C)n1cc(C(=O)NN2CCN(C[C@@H](O)COc3ccc(Cl)c(F)c3)CC2)c(=O)c2cc(F)c(Cl)cc21] |
| 70 | 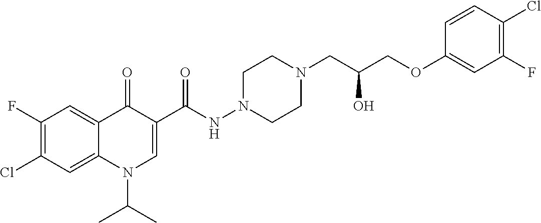 [SMILES: CC(C)n1cc(C(=O)NN2CCN(C[C@H](O)COc3ccc(Cl)c(F)c3)CC2)c(=O)c2cc(F)c(Cl)cc21] |
| 71 | 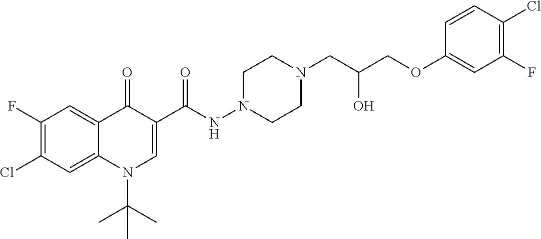 [SMILES: CC(C)(C)n1cc(C(=O)NN2CCN(CC(O)COc3ccc(Cl)c(F)c3)CC2)c(=O)c2cc(F)c(Cl)cc21] |
| 72 | 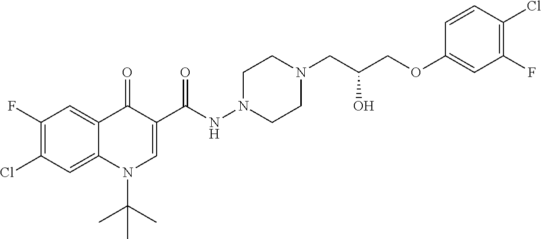 [SMILES: CC(C)(C)n1cc(C(=O)NN2CCN(C[C@@H](O)COc3ccc(Cl)c(F)c3)CC2)c(=O)c2cc(F)c(Cl)cc21] |
| 73 | 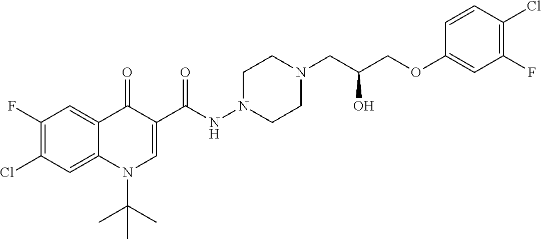 [SMILES: CC(C)(C)n1cc(C(=O)NN2CCN(C[C@H](O)COc3ccc(Cl)c(F)c3)CC2)c(=O)c2cc(F)c(Cl)cc21] |
| 74 | 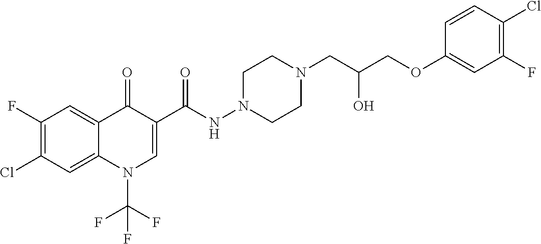 [SMILES: O=C(NN1CCN(CC(O)COc2ccc(Cl)c(F)c2)CC1)c1cn(C(F)(F)F)c2cc(Cl)c(F)cc2c1=O] |
| 75 | 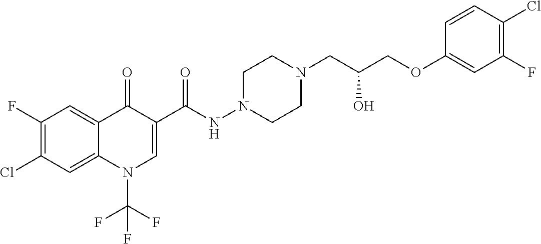 [SMILES: O=C(NN1CCN(C[C@@H](O)COc2ccc(Cl)c(F)c2)CC1)c1cn(C(F)(F)F)c2cc(Cl)c(F)cc2c1=O] |
| 76 | 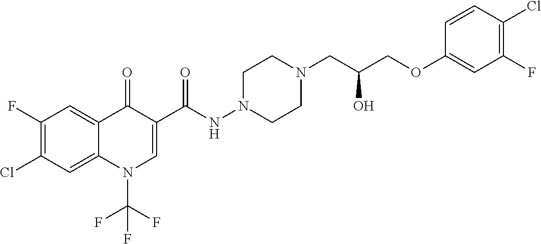 [SMILES: O=C(NN1CCN(C[C@H](O)COc2ccc(Cl)c(F)c2)CC1)c1cn(C(F)(F)F)c2cc(Cl)c(F)cc2c1=O] |
| 77 | 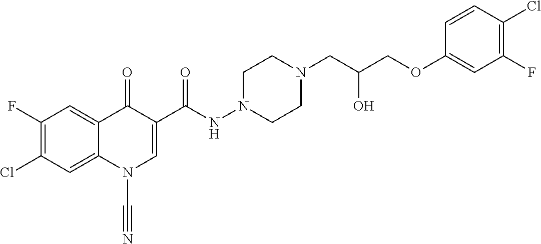 [SMILES: N#Cn1cc(C(=O)NN2CCN(CC(O)COc3ccc(Cl)c(F)c3)CC2)c(=O)c2cc(F)c(Cl)cc21] |
| 78 | 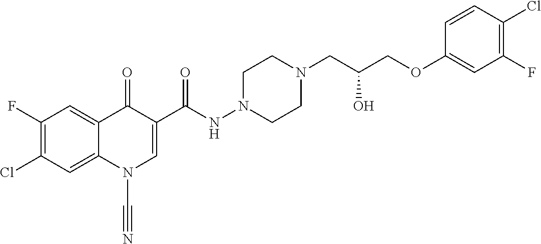 [SMILES: N#Cn1cc(C(=O)NN2CCN(C[C@@H](O)COc3ccc(Cl)c(F)c3)CC2)c(=O)c2cc(F)c(Cl)cc21] |
| 79 | 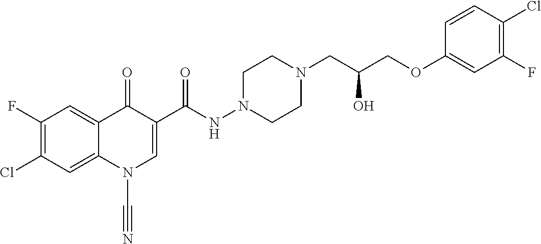 [SMILES: N#Cn1cc(C(=O)NN2CCN(C[C@H](O)COc3ccc(Cl)c(F)c3)CC2)c(=O)c2cc(F)c(Cl)cc21] |
| 80 | 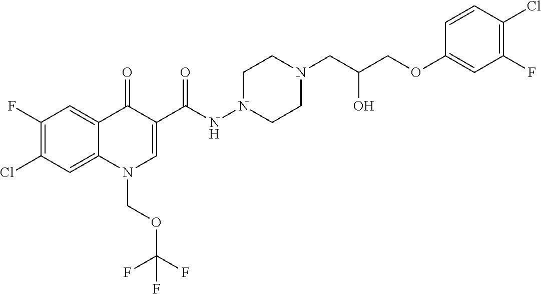 [SMILES: O=C(NN1CCN(CC(O)COc2ccc(Cl)c(F)c2)CC1)c1cn(COC(F)(F)F)c2cc(Cl)c(F)cc2c1=O] |
| 81 | 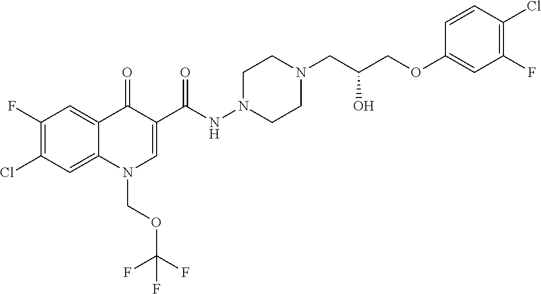 [SMILES: O=C(NN1CCN(C[C@@H](O)COc2ccc(Cl)c(F)c2)CC1)c1cn(COC(F)(F)F)c2cc(Cl)c(F)cc2c1=O] |
| 82 | 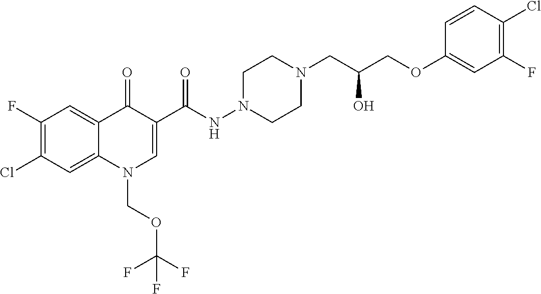 [SMILES: O=C(NN1CCN(C[C@H](O)COc2ccc(Cl)c(F)c2)CC1)c1cn(COC(F)(F)F)c2cc(Cl)c(F)cc2c1=O] |
| 83 | 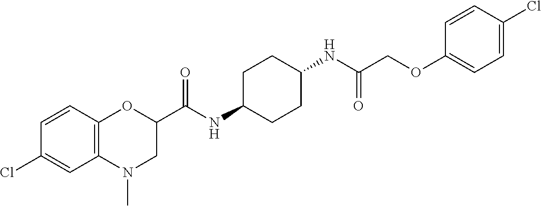 [SMILES: CN1CC(C(=O)N[C@H]2CC[C@H](NC(=O)COc3ccc(Cl)cc3)CC2)Oc2ccc(Cl)cc21] |
| 84 | 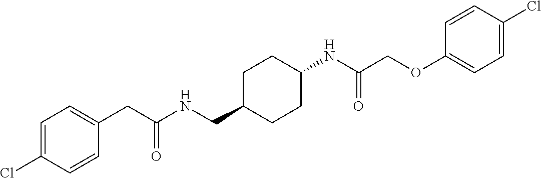 [SMILES: O=C(Cc1ccc(Cl)cc1)NC[C@H]1CC[C@H](NC(=O)COc2ccc(Cl)cc2)CC1] |
| 85 | 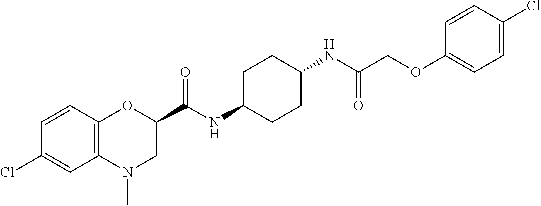 [SMILES: CN1C[C@H](C(=O)N[C@H]2CC[C@H](NC(=O)COc3ccc(Cl)cc3)CC2)Oc2ccc(Cl)cc21] |
| 86 | 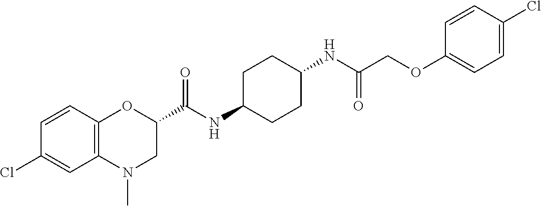 [SMILES: CN1C[C@@H](C(=O)N[C@H]2CC[C@H](NC(=O)COc3ccc(Cl)cc3)CC2)Oc2ccc(Cl)cc21] |
| 87 | 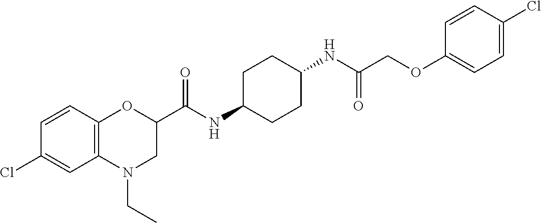 [SMILES: CCN1CC(C(=O)N[C@H]2CC[C@H](NC(=O)COc3ccc(Cl)cc3)CC2)Oc2ccc(Cl)cc21] |
| 88 | 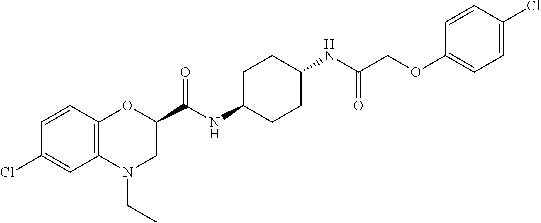 [SMILES: CCN1C[C@H](C(=O)N[C@H]2CC[C@H](NC(=O)COc3ccc(Cl)cc3)CC2)Oc2ccc(Cl)cc21] |
| 89 | 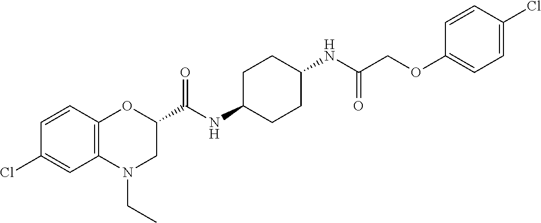 [SMILES: CCN1C[C@@H](C(=O)N[C@H]2CC[C@H](NC(=O)COc3ccc(Cl)cc3)CC2)Oc2ccc(Cl)cc21] |
| 90 | 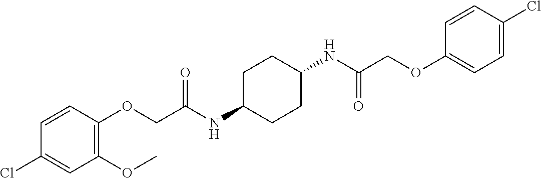 [SMILES: COc1cc(Cl)ccc1OCC(=O)N[C@H]1CC[C@H](NC(=O)COc2ccc(Cl)cc2)CC1] |
| 91 | 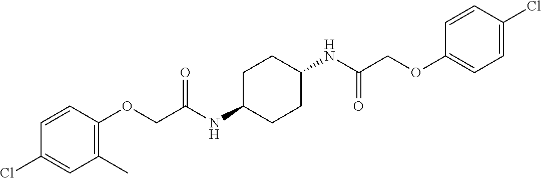 [SMILES: Cc1cc(Cl)ccc1OCC(=O)N[C@H]1CC[C@H](NC(=O)COc2ccc(Cl)cc2)CC1] |
| 92 | 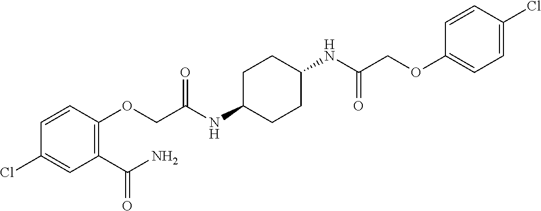 [SMILES: NC(=O)c1cc(Cl)ccc1OCC(=O)N[C@H]1CC[C@H](NC(=O)COc2ccc(Cl)cc2)CC1] |
| 93 | 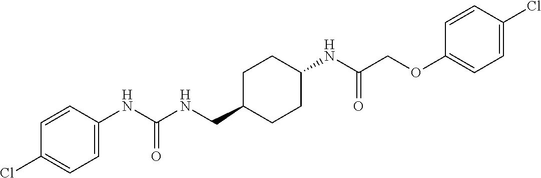 [SMILES: O=C(NC[C@H]1CC[C@H](NC(=O)COc2ccc(Cl)cc2)CC1)Nc1ccc(Cl)cc1] |
| 94 | 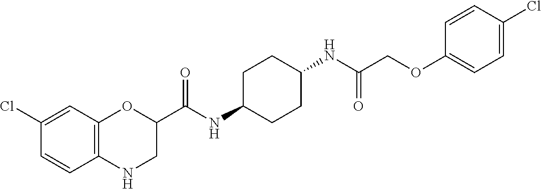 [SMILES: O=C(COc1ccc(Cl)cc1)N[C@H]1CC[C@H](NC(=O)C2CNc3ccc(Cl)cc3O2)CC1] |
| 95 | 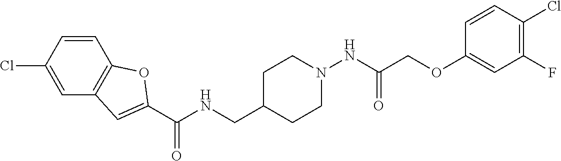 [SMILES: O=C(COc1ccc(Cl)c(F)c1)NN1CCC(CNC(=O)c2cc3cc(Cl)ccc3o2)CC1] |
| 96 | 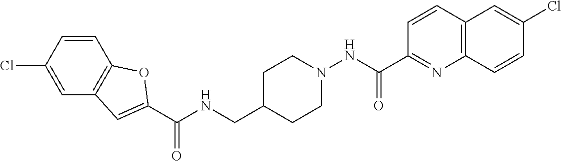 [SMILES: O=C(NN1CCC(CNC(=O)c2cc3cc(Cl)ccc3o2)CC1)c1ccc2cc(Cl)ccc2n1] |
| 97 | 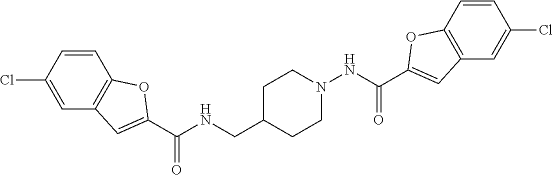 [SMILES: O=C(NCC1CCN(NC(=O)c2cc3cc(Cl)ccc3o2)CC1)c1cc2cc(Cl)ccc2o1] |
| 98 | 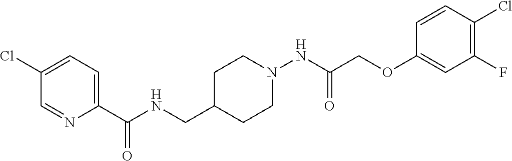 [SMILES: O=C(COc1ccc(Cl)c(F)c1)NN1CCC(CNC(=O)c2ccc(Cl)cn2)CC1] |
| 99 | 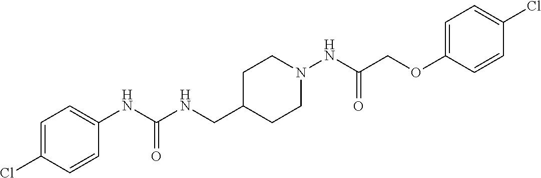 [SMILES: O=C(COc1ccc(Cl)cc1)NN1CCC(CNC(=O)Nc2ccc(Cl)cc2)CC1] |
| 100 | 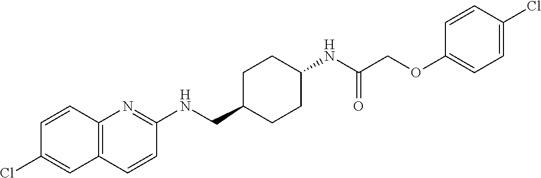 [SMILES: O=C(COc1ccc(Cl)cc1)N[C@H]1CC[C@H](CNc2ccc3cc(Cl)ccc3n2)CC1] |
| 101 | 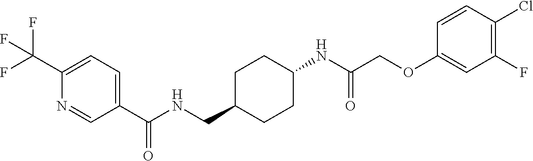 [SMILES: O=C(COc1ccc(Cl)c(F)c1)N[C@H]1CC[C@H](CNC(=O)c2ccc(C(F)(F)F)nc2)CC1] |
| 102 | 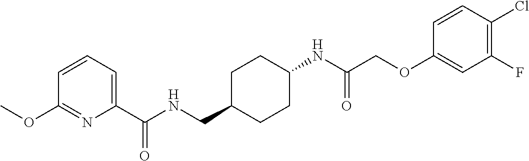 [SMILES: COc1cccc(C(=O)NC[C@H]2CC[C@H](NC(=O)COc3ccc(Cl)c(F)c3)CC2)n1] |
| 103 | 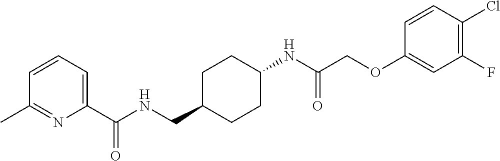 [SMILES: Cc1cccc(C(=O)NC[C@H]2CC[C@H](NC(=O)COc3ccc(Cl)c(F)c3)CC2)n1] |
| 104 | 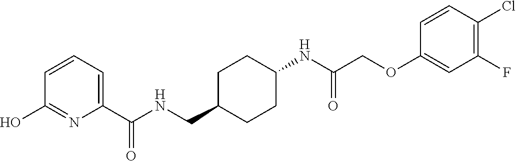 [SMILES: O=C(COc1ccc(Cl)c(F)c1)N[C@H]1CC[C@H](CNC(=O)c2cccc(O)n2)CC1] |
| 105 | 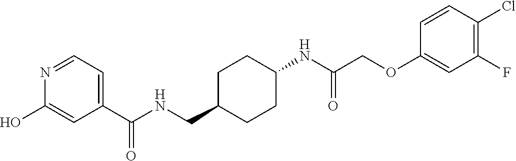 [SMILES: O=C(COc1ccc(Cl)c(F)c1)N[C@H]1CC[C@H](CNC(=O)c2ccnc(O)c2)CC1] |
| 106 | 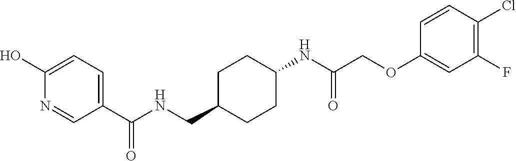 [SMILES: O=C(COc1ccc(Cl)c(F)c1)N[C@H]1CC[C@H](CNC(=O)c2ccc(O)nc2)CC1] |
| 107 | 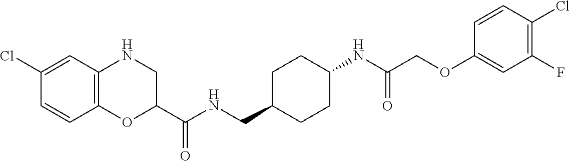 [SMILES: O=C(COc1ccc(Cl)c(F)c1)N[C@H]1CC[C@H](CNC(=O)C2CNc3cc(Cl)ccc3O2)CC1] |
| 108 | 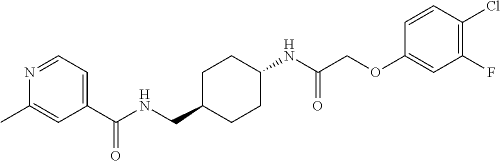 [SMILES: Cc1cc(C(=O)NC[C@H]2CC[C@H](NC(=O)COc3ccc(Cl)c(F)c3)CC2)ccn1] |
| 109 | 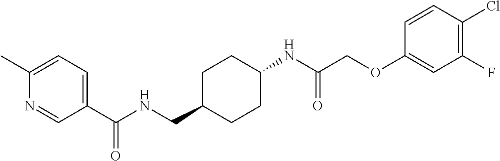 [SMILES: Cc1ccc(C(=O)NC[C@H]2CC[C@H](NC(=O)COc3ccc(Cl)c(F)c3)CC2)cn1] |
| 110 | 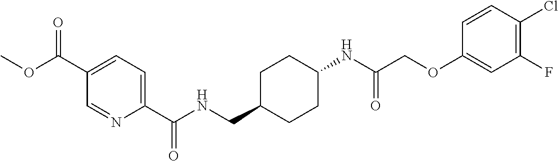 [SMILES: COC(=O)c1ccc(C(=O)NC[C@H]2CC[C@H](NC(=O)COc3ccc(Cl)c(F)c3)CC2)nc1] |
| 111 | 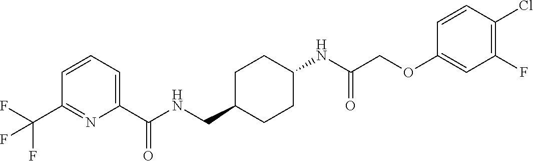 [SMILES: O=C(COc1ccc(Cl)c(F)c1)N[C@H]1CC[C@H](CNC(=O)c2cccc(C(F)(F)F)n2)CC1] |
| 112 | 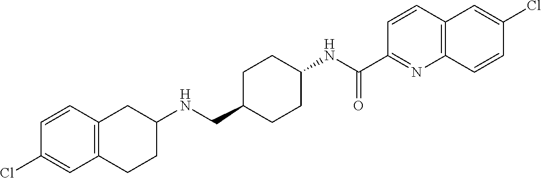 [SMILES: O=C(N[C@H]1CC[C@H](CNC2CCc3cc(Cl)ccc3C2)CC1)c1ccc2cc(Cl)ccc2n1] |
| 113 | 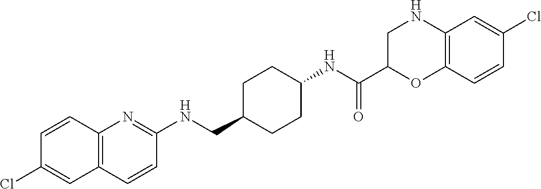 [SMILES: O=C(N[C@H]1CC[C@H](CNc2ccc3cc(Cl)ccc3n2)CC1)C1CNc2cc(Cl)ccc2O1] |
| 114 | 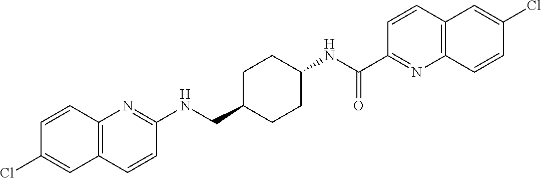 [SMILES: O=C(N[C@H]1CC[C@H](CNc2ccc3cc(Cl)ccc3n2)CC1)c1ccc2cc(Cl)ccc2n1] |

In some embodiments of the compounds of formulae (I), (II), (III), (IV), (V), (VI), (XI), (XII), (XIII), (XIV), (XIX), (A-1), (B-1), (C-1), (D-1), (E-1), and (F-1), or the salts thereof, the compound is other than the compounds in Table 1X and Table 1XX or a salt thereof. In some embodiments of the compounds of formulae (I), (II), (III), (IV), (V), (VI), (XI), (XII), (XIII), (XIV), (XIX), (A-1), (B-1), (C-1), (D-1), (E-1), and (F-1), or the salts thereof, the compound is other than the Compound Nos. X1 to X-166 in Table 1X and the Compound Nos. XX-1 to XX-74 or a salt thereof.

TABLE 1X

| Compound | |
|---|---|
| No. | Structure |
| X-1 | 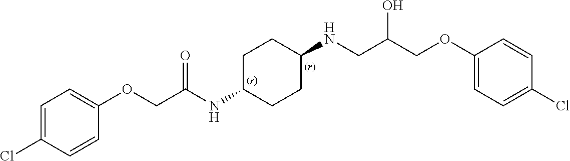 [SMILES: O=C(COc1ccc(Cl)cc1)N[C@H]1CC[C@H](NCC(O)COc2ccc(Cl)cc2)CC1] |
| X-2 | 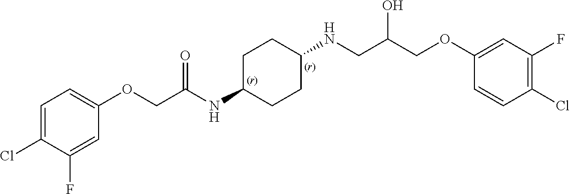 [SMILES: O=C(COc1ccc(Cl)c(F)c1)N[C@H]1CC[C@H](NCC(O)COc2ccc(Cl)c(F)c2)CC1] |
| X-3 | 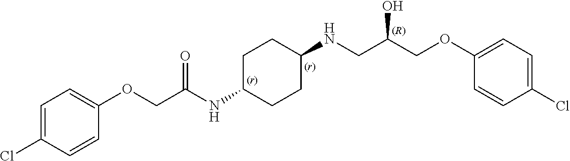 |
| X-4 | 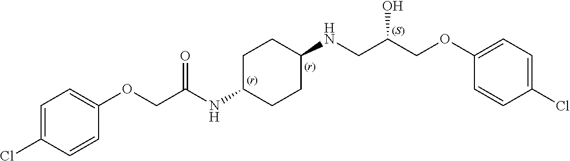 |
| X-5 | 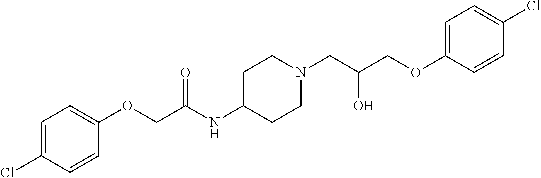 [SMILES: O=C(COc1ccc(Cl)cc1)NC1CCN(CC(O)COc2ccc(Cl)cc2)CC1] |
| X-6 | 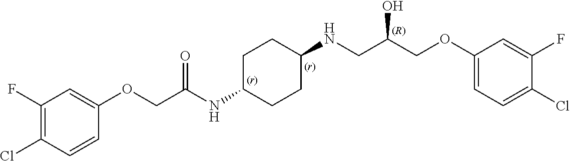 |
| X-7 | 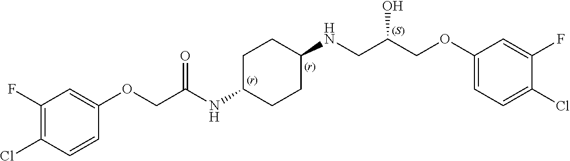 |
| X-8 | 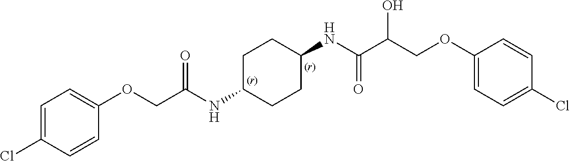 [SMILES: O=C(COc1ccc(Cl)cc1)N[C@H]1CC[C@H](NC(=O)C(O)COc2ccc(Cl)cc2)CC1] |
| X-9 | 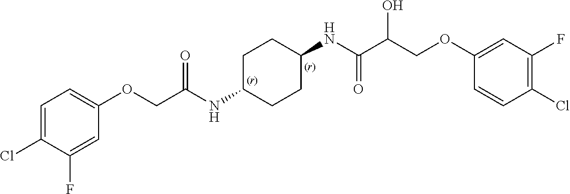 [SMILES: O=C(COc1ccc(Cl)c(F)c1)N[C@H]1CC[C@H](NC(=O)C(O)COc2ccc(Cl)c(F)c2)CC1] |
| X-10 | 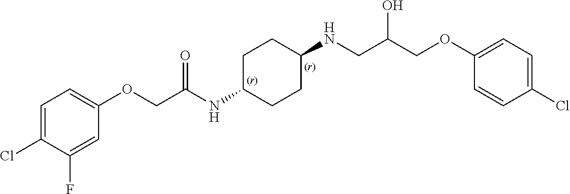 [SMILES: O=C(COc1ccc(Cl)c(F)c1)N[C@H]1CC[C@H](NCC(O)COc2ccc(Cl)cc2)CC1] |
| X-11 | 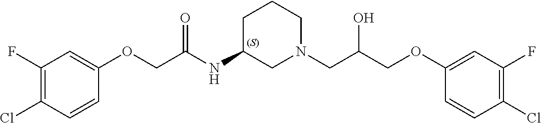 |
| X-12 | 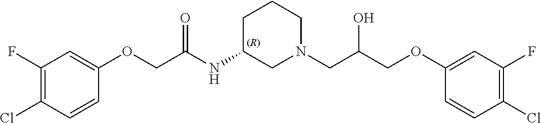 |
| X-13 | 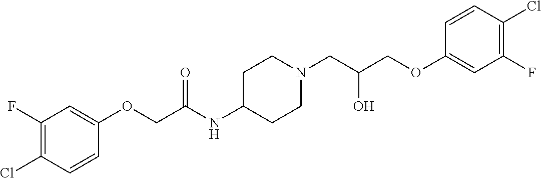 [SMILES: O=C(COc1ccc(Cl)c(F)c1)NC1CCN(CC(O)COc2ccc(Cl)c(F)c2)CC1] |
| X-14 | 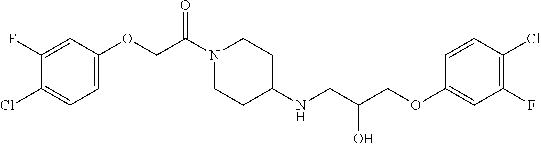 [SMILES: O=C(COc1ccc(Cl)c(F)c1)N1CCC(NCC(O)COc2ccc(Cl)c(F)c2)CC1] |
| X-15 | 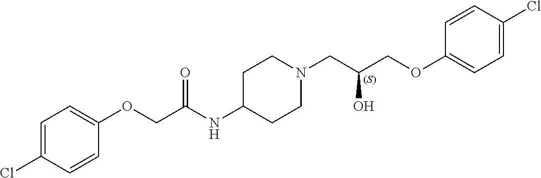 |
| X-16 | 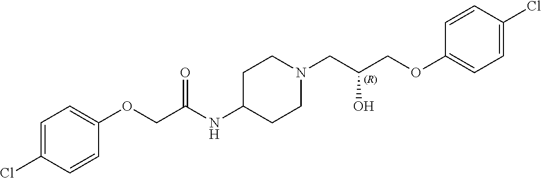 |
| X-17 | 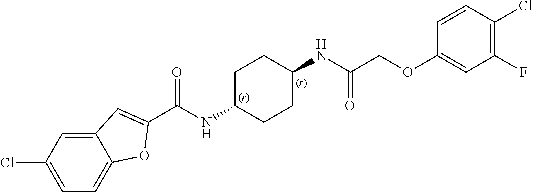 [SMILES: O=C(COc1ccc(Cl)c(F)c1)N[C@H]1CC[C@H](NC(=O)c2cc3cc(Cl)ccc3o2)CC1] |
| X-18 | 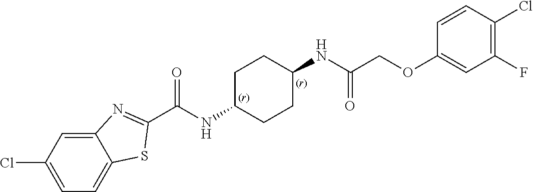 [SMILES: O=C(COc1ccc(Cl)c(F)c1)N[C@H]1CC[C@H](NC(=O)c2nc3cc(Cl)ccc3s2)CC1] |
| X-19 | 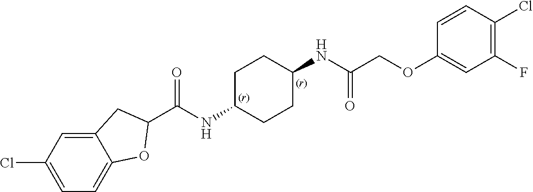 [SMILES: O=C(COc1ccc(Cl)c(F)c1)N[C@H]1CC[C@H](NC(=O)C2Cc3cc(Cl)ccc3O2)CC1] |
| X-20 | 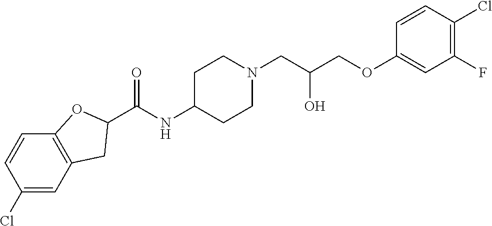 [SMILES: O=C(NC1CCN(CC(O)COc2ccc(Cl)c(F)c2)CC1)C1Cc2cc(Cl)ccc2O1] |
| X-21 | 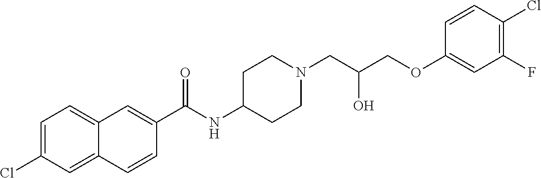 [SMILES: O=C(NC1CCN(CC(O)COc2ccc(Cl)c(F)c2)CC1)c1ccc2cc(Cl)ccc2c1] |
| X-22 | 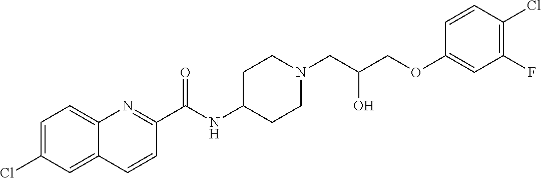 [SMILES: O=C(NC1CCN(CC(O)COc2ccc(Cl)c(F)c2)CC1)c1ccc2cc(Cl)ccc2n1] |
| X-23 | 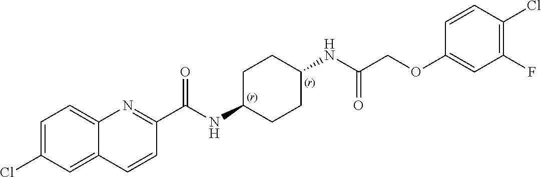 [SMILES: O=C(COc1ccc(Cl)c(F)c1)N[C@H]1CC[C@H](NC(=O)c2ccc3cc(Cl)ccc3n2)CC1] |
| X-24 | 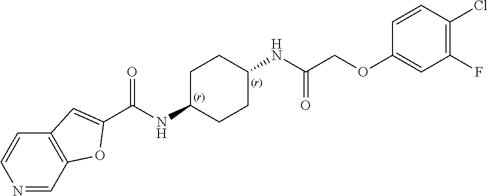 [SMILES: O=C(COc1ccc(Cl)c(F)c1)N[C@H]1CC[C@H](NC(=O)c2cc3ccncc3o2)CC1] |
| X-25 | 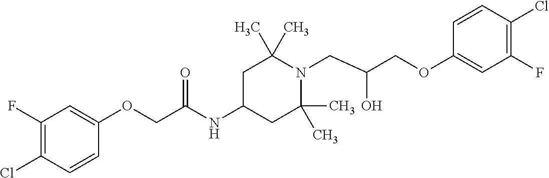 [SMILES: CC1(C)CC(NC(=O)COc2ccc(Cl)c(F)c2)CC(C)(C)N1CC(O)COc1ccc(Cl)c(F)c1] |
| X-26 | 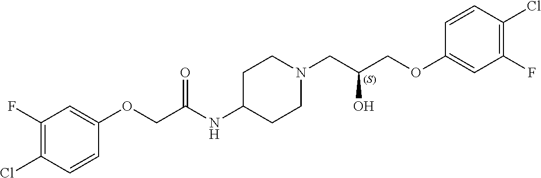 |
| X-27 | 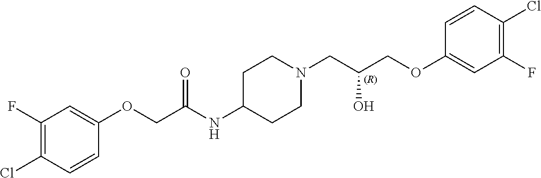 |
| X-28 | 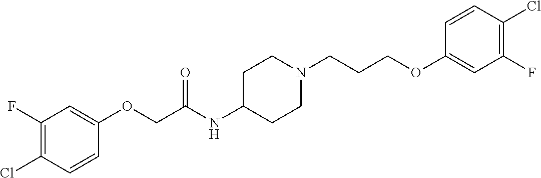 [SMILES: O=C(COc1ccc(Cl)c(F)c1)NC1CCN(CCCOc2ccc(Cl)c(F)c2)CC1] |
| X-29 | 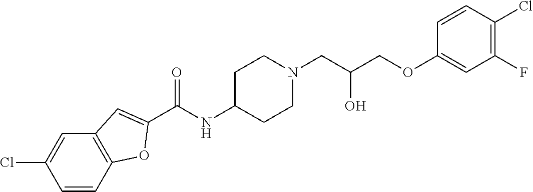 [SMILES: O=C(NC1CCN(CC(O)COc2ccc(Cl)c(F)c2)CC1)c1cc2cc(Cl)ccc2o1] |
| X-30 | 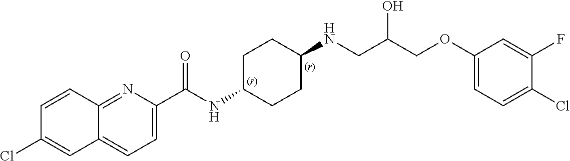 [SMILES: O=C(N[C@H]1CC[C@H](NCC(O)COc2ccc(Cl)c(F)c2)CC1)c1ccc2cc(Cl)ccc2n1] |
| X-31 | 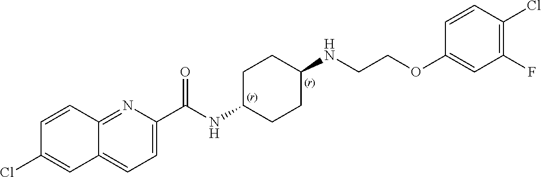 [SMILES: O=C(N[C@H]1CC[C@H](NCCOc2ccc(Cl)c(F)c2)CC1)c1ccc2cc(Cl)ccc2n1] |
| X-32 | 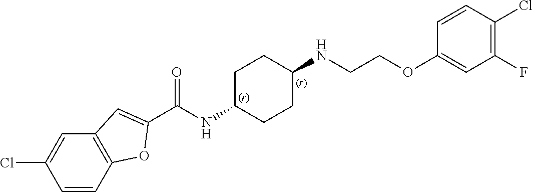 [SMILES: O=C(N[C@H]1CC[C@H](NCCOc2ccc(Cl)c(F)c2)CC1)c1cc2cc(Cl)ccc2o1] |
| X-33 | 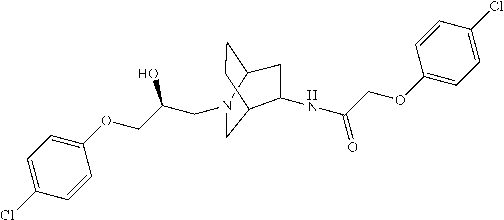 [SMILES: O=C(COc1ccc(Cl)cc1)NC1CC2CCC1CN2C[C@H](O)COc1ccc(Cl)cc1] |
| X-34 | 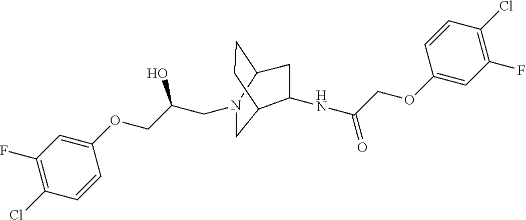 [SMILES: O=C(COc1ccc(Cl)c(F)c1)NC1CC2CCC1CN2C[C@H](O)COc1ccc(Cl)c(F)c1] |
| X-35 | 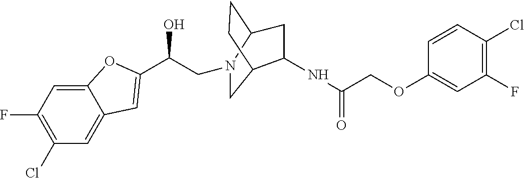 [SMILES: O=C(COc1ccc(Cl)c(F)c1)NC1CC2CCC1CN2C[C@H](O)c1cc2cc(Cl)c(F)cc2o1] |
| X-36 | 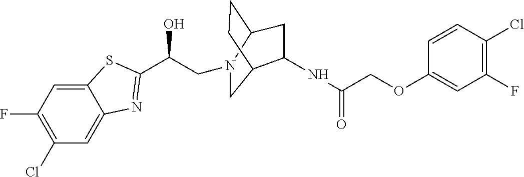 [SMILES: O=C(COc1ccc(Cl)c(F)c1)NC1CC2CCC1CN2C[C@H](O)c1nc2cc(Cl)c(F)cc2s1] |
| X-37 | 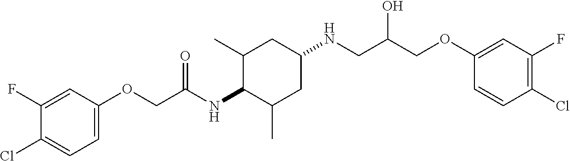 |
| X-38 | 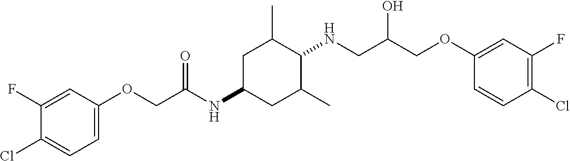 |
| X-39 | 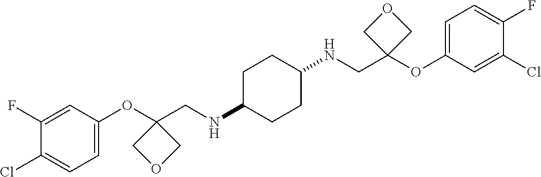 |
| X-40 | 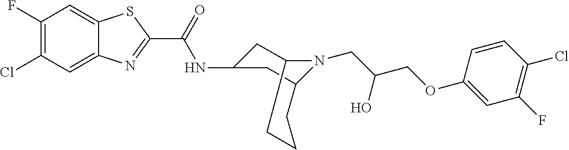 [SMILES: O=C(NC1CC2CCCC(C1)N2CC(O)COc1ccc(Cl)c(F)c1)c1nc2cc(Cl)c(F)cc2s1] |
| X-41 | 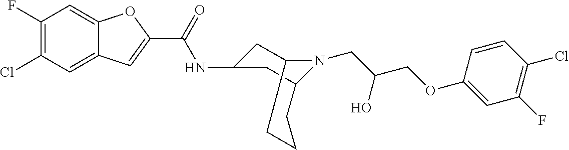 [SMILES: O=C(NC1CC2CCCC(C1)N2CC(O)COc1ccc(Cl)c(F)c1)c1cc2cc(Cl)c(F)cc2o1] |
| X-42 | 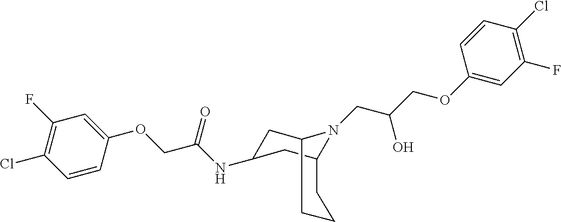 [SMILES: O=C(COc1ccc(Cl)c(F)c1)NC1CC2CCCC(C1)N2CC(O)COc1ccc(Cl)c(F)c1] |
| X-43 | 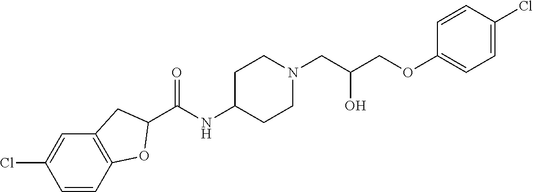 [SMILES: O=C(NC1CCN(CC(O)COc2ccc(Cl)cc2)CC1)C1Cc2cc(Cl)ccc2O1] |
| X-44 | 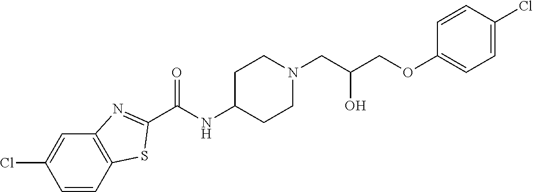 [SMILES: O=C(NC1CCN(CC(O)COc2ccc(Cl)cc2)CC1)c1nc2cc(Cl)ccc2s1] |
| X-45 | 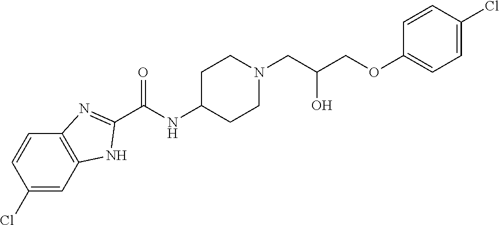 [SMILES: O=C(NC1CCN(CC(O)COc2ccc(Cl)cc2)CC1)c1nc2ccc(Cl)cc2[nH]1] |
| X-46 | 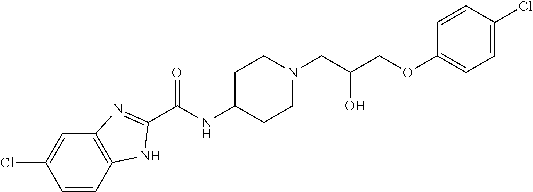 [SMILES: O=C(NC1CCN(CC(O)COc2ccc(Cl)cc2)CC1)c1nc2cc(Cl)ccc2[nH]1] |
| X-47 | 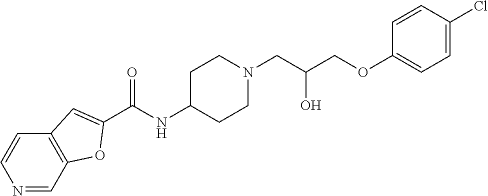 [SMILES: O=C(NC1CCN(CC(O)COc2ccc(Cl)cc2)CC1)c1cc2ccncc2o1] |
| X-48 | 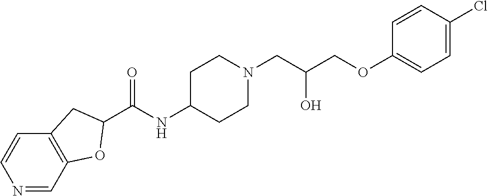 [SMILES: O=C(NC1CCN(CC(O)COc2ccc(Cl)cc2)CC1)C1Cc2ccncc2O1] |
| X-49 | 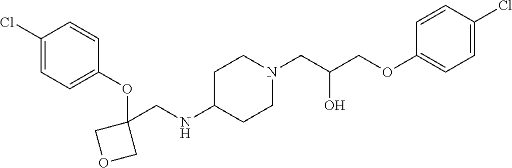 [SMILES: OC(COc1ccc(Cl)cc1)CN1CCC(NCC2(Oc3ccc(Cl)cc3)COC2)CC1] |
| X-50 | 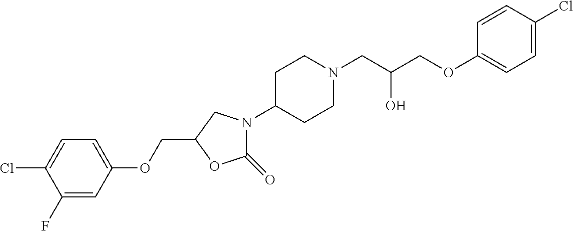 [SMILES: O=C1OC(COc2ccc(Cl)c(F)c2)CN1C1CCN(CC(O)COc2ccc(Cl)cc2)CC1] |
| X-51 | 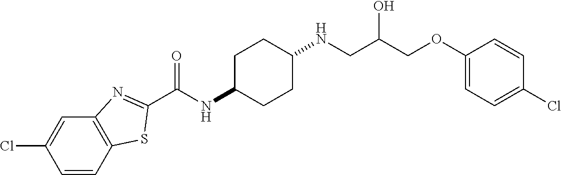 |
| X-52 | 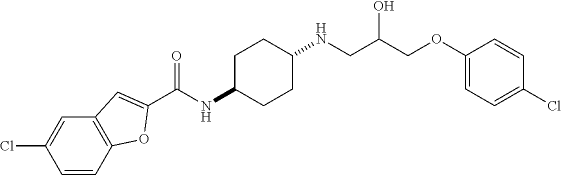 |
| X-53 | 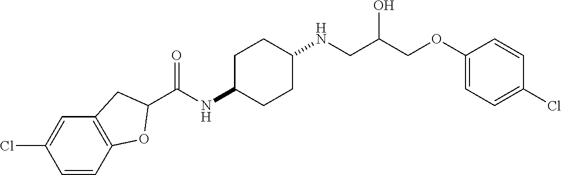 |
| X-54 | 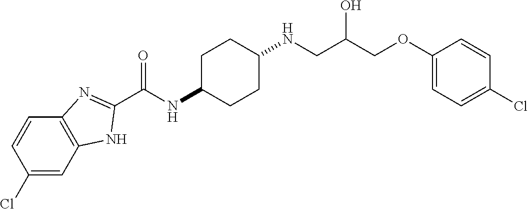 |
| X-55 | 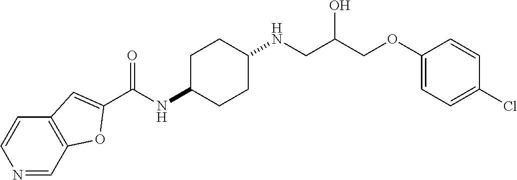 |
| X-56 | 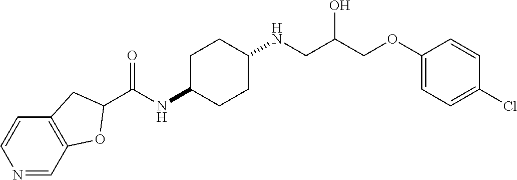 |
| X-57 | 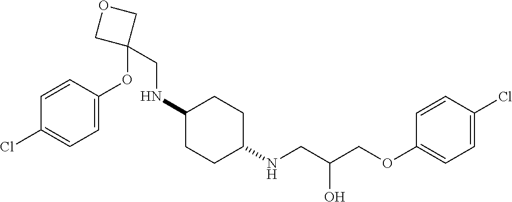 |
| X-58 | 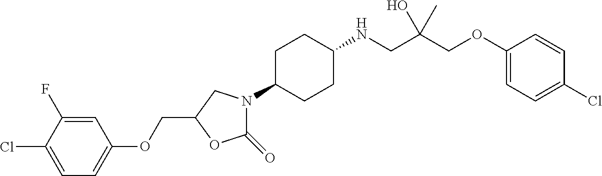 [SMILES: CC(O)(CN[C@H]1CC[C@H](N2CC(COc3ccc(Cl)c(F)c3)OC2=O)CC1)COc1ccc(Cl)cc1] |
| X-59 | 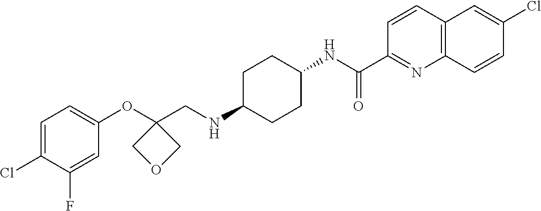 [SMILES: O=C(N[C@H]1CC[C@H](NCC2(Oc3ccc(Cl)c(F)c3)COC2)CC1)c1ccc2cc(Cl)ccc2n1] |
| X-60 | 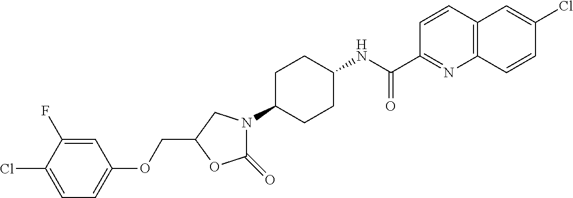 [SMILES: O=C(N[C@H]1CC[C@H](N2CC(COc3ccc(Cl)c(F)c3)OC2=O)CC1)c1ccc2cc(Cl)ccc2n1] |
| X-61 | 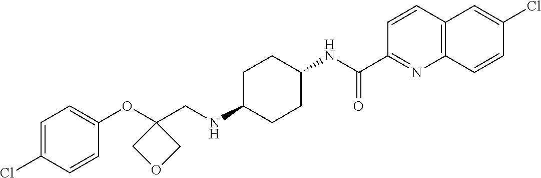 [SMILES: O=C(N[C@H]1CC[C@H](NCC2(Oc3ccc(Cl)cc3)COC2)CC1)c1ccc2cc(Cl)ccc2n1] |
| X-62 | 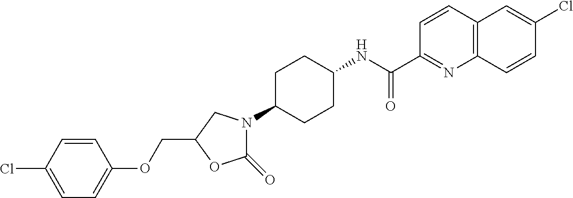 [SMILES: O=C(N[C@H]1CC[C@H](N2CC(COc3ccc(Cl)cc3)OC2=O)CC1)c1ccc2cc(Cl)ccc2n1] |
| X-63 | 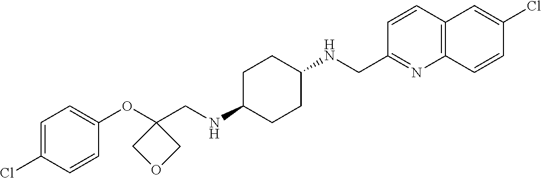 [SMILES: Clc1ccc(OC2(CN[C@H]3CC[C@H](NCc4ccc5cc(Cl)ccc5n4)CC3)COC2)cc1] |
| X-64 | 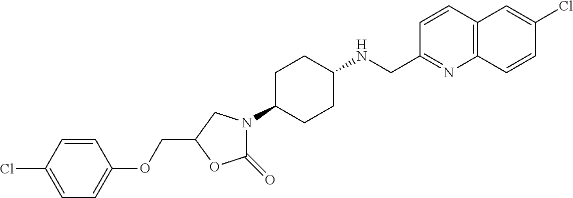 [SMILES: O=C1OC(COc2ccc(Cl)cc2)CN1[C@H]1CC[C@H](NCc2ccc3cc(Cl)ccc3n2)CC1] |
| X-65 | 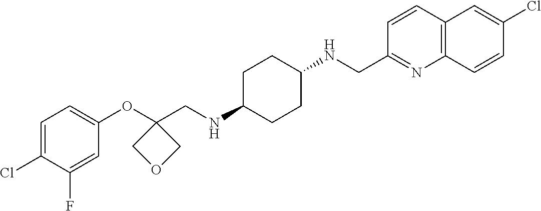 [SMILES: Fc1cc(OC2(CN[C@H]3CC[C@H](NCc4ccc5cc(Cl)ccc5n4)CC3)COC2)ccc1Cl] |
| X-66 | 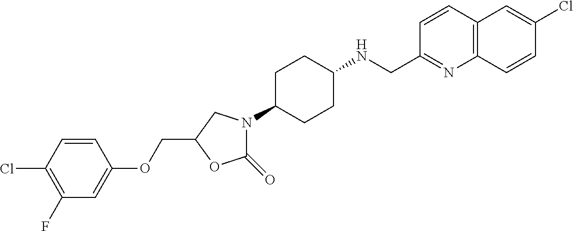 [SMILES: O=C1OC(COc2ccc(Cl)c(F)c2)CN1[C@H]1CC[C@H](NCc2ccc3cc(Cl)ccc3n2)CC1] |
| X-67 | 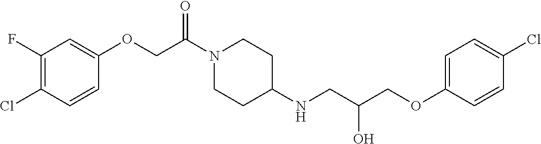 [SMILES: O=C(COc1ccc(Cl)c(F)c1)N1CCC(NCC(O)COc2ccc(Cl)cc2)CC1] |
| X-68 | 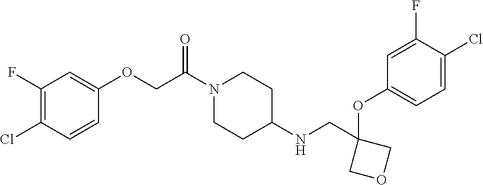 [SMILES: O=C(COc1ccc(Cl)c(F)c1)N1CCC(NCC2(Oc3ccc(Cl)c(F)c3)COC2)CC1] |
| X-69 | 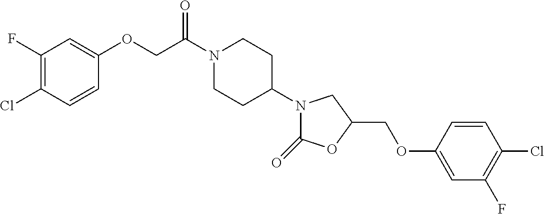 [SMILES: O=C(COc1ccc(Cl)c(F)c1)N1CCC(N2CC(COc3ccc(Cl)c(F)c3)OC2=O)CC1] |
| X-70 | 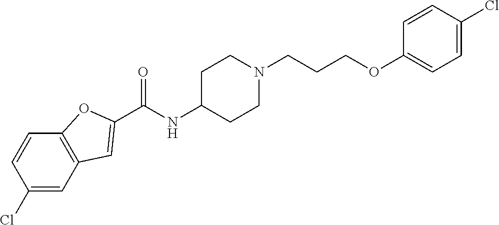 [SMILES: O=C(NC1CCN(CCCOc2ccc(Cl)cc2)CC1)c1cc2cc(Cl)ccc2o1] |
| X-71 | 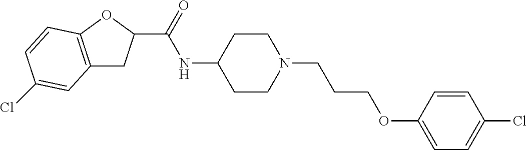 [SMILES: O=C(NC1CCN(CCCOc2ccc(Cl)cc2)CC1)C1Cc2cc(Cl)ccc2O1] |
| X-72 | 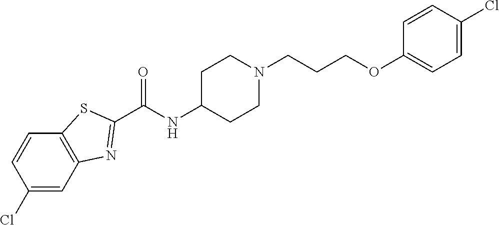 [SMILES: O=C(NC1CCN(CCCOc2ccc(Cl)cc2)CC1)c1nc2cc(Cl)ccc2s1] |
| X-73 | 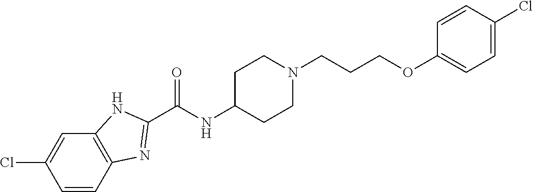 [SMILES: O=C(NC1CCN(CCCOc2ccc(Cl)cc2)CC1)c1nc2ccc(Cl)cc2[nH]1] |
| X-74 | 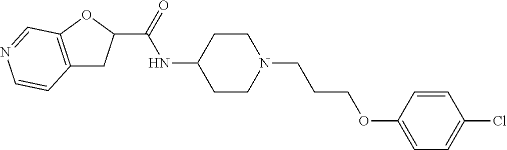 [SMILES: O=C(NC1CCN(CCCOc2ccc(Cl)cc2)CC1)C1Cc2ccncc2O1] |
| X-75 | 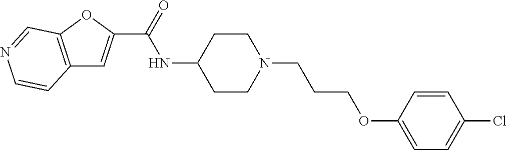 [SMILES: O=C(NC1CCN(CCCOc2ccc(Cl)cc2)CC1)c1cc2ccncc2o1] |
| X-76 | 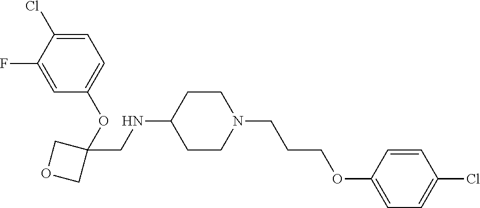 [SMILES: Fc1cc(OC2(CNC3CCN(CCCOc4ccc(Cl)cc4)CC3)COC2)ccc1Cl] |
| X-77 | 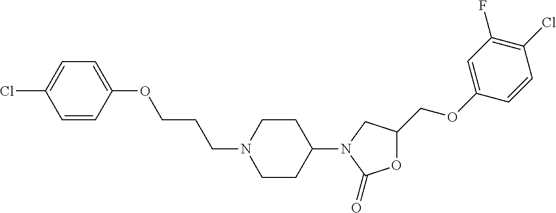 [SMILES: O=C1OC(COc2ccc(Cl)c(F)c2)CN1C1CCN(CCCOc2ccc(Cl)cc2)CC1] |
| X-78 | 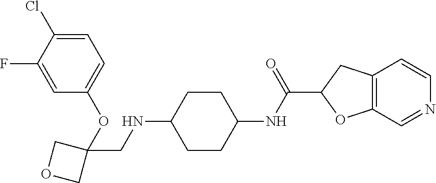 [SMILES: O=C(NC1CCC(NCC2(Oc3ccc(Cl)c(F)c3)COC2)CC1)C1Cc2ccncc2O1] |
| X-79 | 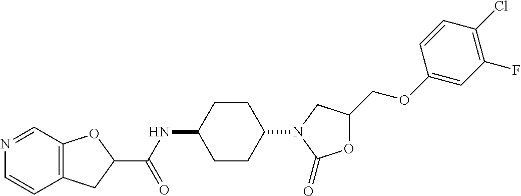 |
| X-80 | 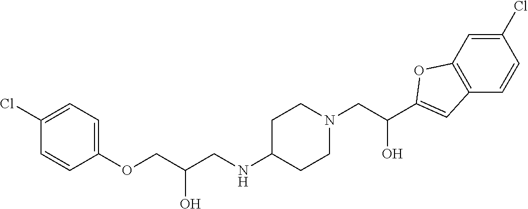 [SMILES: OC(CNC1CCN(CC(O)c2cc3ccc(Cl)cc3o2)CC1)COc1ccc(Cl)cc1] |
| X-81 | 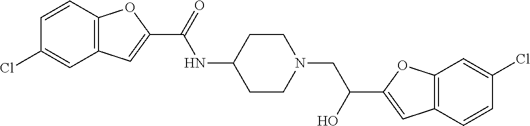 [SMILES: O=C(NC1CCN(CC(O)c2cc3ccc(Cl)cc3o2)CC1)c1cc2cc(Cl)ccc2o1] |
| X-82 | 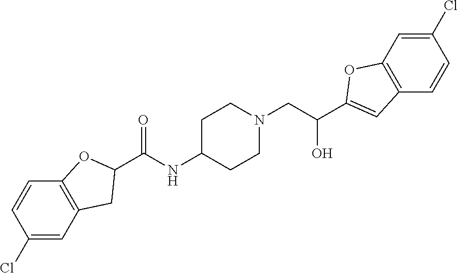 [SMILES: O=C(NC1CCN(CC(O)c2cc3ccc(Cl)cc3o2)CC1)C1Cc2cc(Cl)ccc2O1] |
| X-83 | 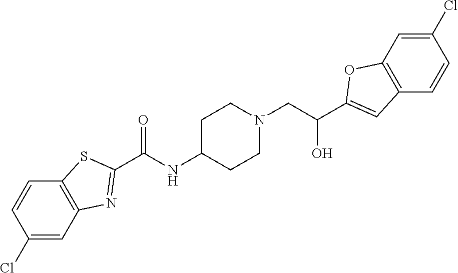 [SMILES: O=C(NC1CCN(CC(O)c2cc3ccc(Cl)cc3o2)CC1)c1nc2cc(Cl)ccc2s1] |
| X-84 | 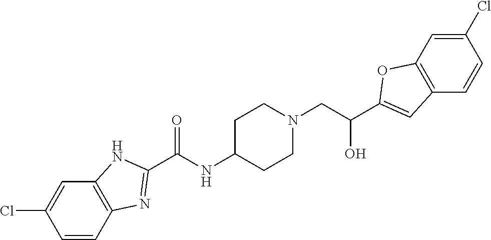 [SMILES: O=C(NC1CCN(CC(O)c2cc3ccc(Cl)cc3o2)CC1)c1nc2ccc(Cl)cc2[nH]1] |
| X-85 | 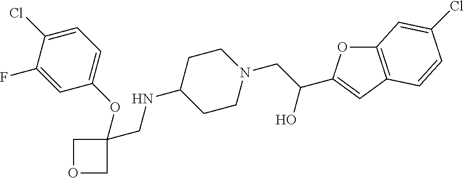 [SMILES: OC(CN1CCC(NCC2(Oc3ccc(Cl)c(F)c3)COC2)CC1)c1cc2ccc(Cl)cc2o1] |
| X-86 | 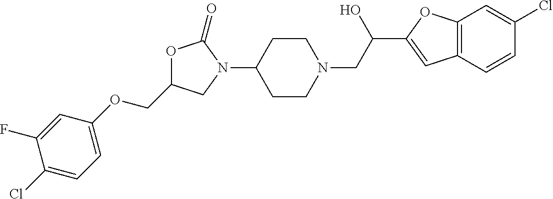 [SMILES: O=C1OC(COc2ccc(Cl)c(F)c2)CN1C1CCN(CC(O)c2cc3ccc(Cl)cc3o2)CC1] |
| X-87 | 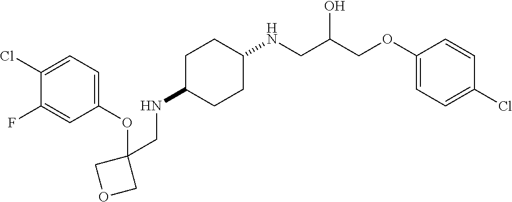 [SMILES: OC(CN[C@H]1CC[C@H](NCC2(Oc3ccc(Cl)c(F)c3)COC2)CC1)COc1ccc(Cl)cc1] |
| X-88 | 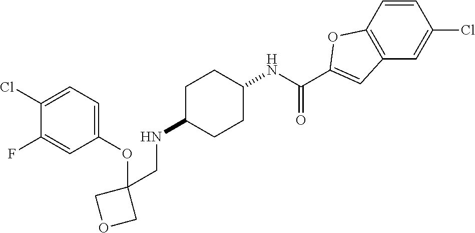 [SMILES: O=C(N[C@H]1CC[C@H](NCC2(Oc3ccc(Cl)c(F)c3)COC2)CC1)c1cc2cc(Cl)ccc2o1] |
| X-89 | 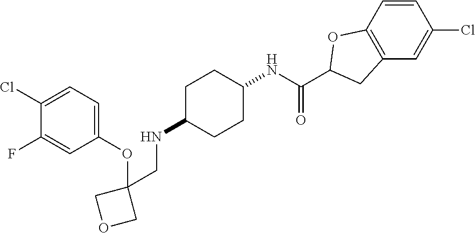 [SMILES: O=C(N[C@H]1CC[C@H](NCC2(Oc3ccc(Cl)c(F)c3)COC2)CC1)C1Cc2cc(Cl)ccc2O1] |
| X-90 | 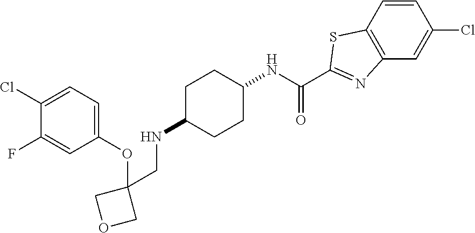 [SMILES: O=C(N[C@H]1CC[C@H](NCC2(Oc3ccc(Cl)c(F)c3)COC2)CC1)c1nc2cc(Cl)ccc2s1] |
| X-91 | 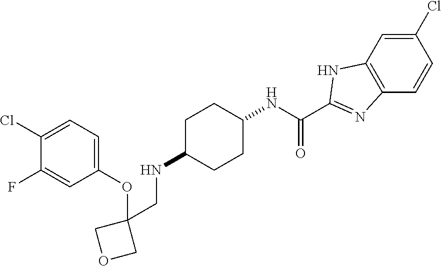 [SMILES: O=C(N[C@H]1CC[C@H](NCC2(Oc3ccc(Cl)c(F)c3)COC2)CC1)c1nc2ccc(Cl)cc2[nH]1] |
| X-92 | 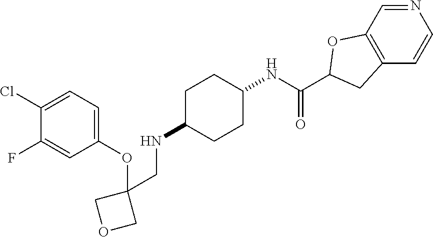 [SMILES: O=C(N[C@H]1CC[C@H](NCC2(Oc3ccc(Cl)c(F)c3)COC2)CC1)C1Cc2ccncc2O1] |
| X-93 | 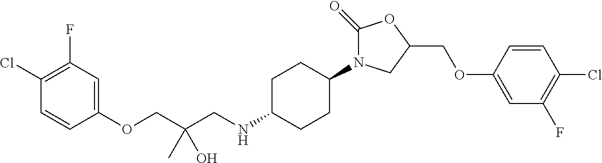 [SMILES: CC(O)(CN[C@H]1CC[C@H](N2CC(COc3ccc(Cl)c(F)c3)OC2=O)CC1)COc1ccc(Cl)c(F)c1] |
| X-94 | 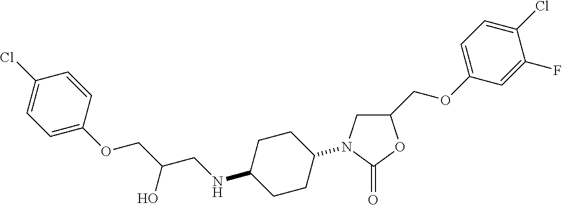 |
| X-95 | 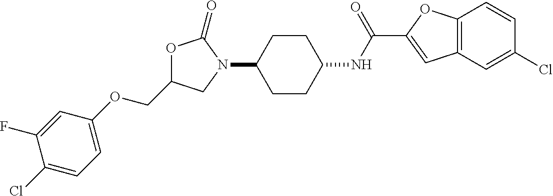 |
| X-96 | 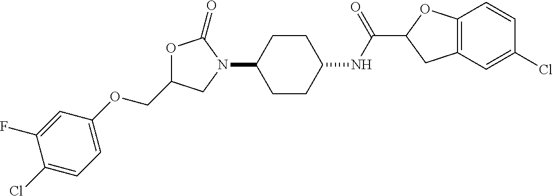 |
| X-97 | 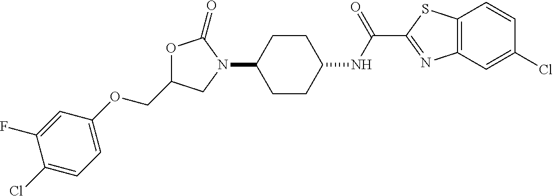 |
| X-98 | 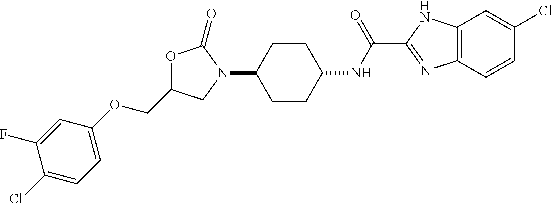 |
| X-99 | 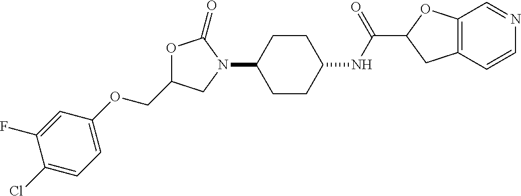 |
| X-100 | 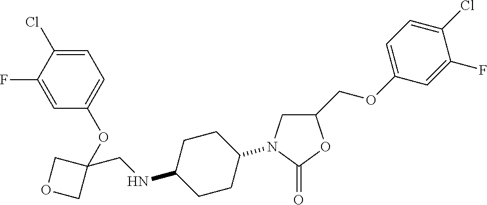 |
| X-101 | 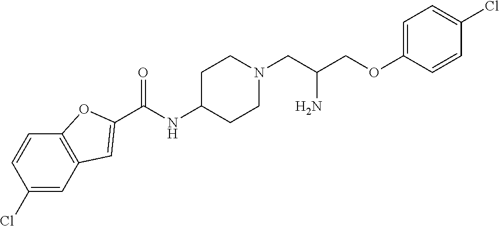 [SMILES: NC(COc1ccc(Cl)cc1)CN1CCC(NC(=O)c2cc3cc(Cl)ccc3o2)CC1] |
| X-102 | 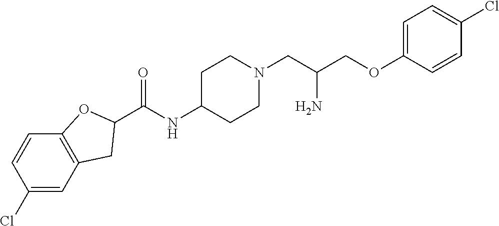 [SMILES: NC(COc1ccc(Cl)cc1)CN1CCC(NC(=O)C2Cc3cc(Cl)ccc3O2)CC1] |
| X-103 | 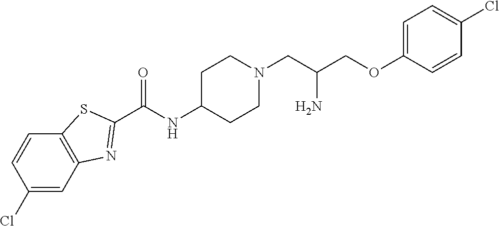 [SMILES: NC(COc1ccc(Cl)cc1)CN1CCC(NC(=O)c2nc3cc(Cl)ccc3s2)CC1] |
| X-104 | 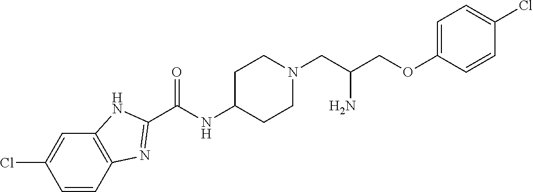 [SMILES: NC(COc1ccc(Cl)cc1)CN1CCC(NC(=O)c2nc3ccc(Cl)cc3[nH]2)CC1] |
| X-105 | 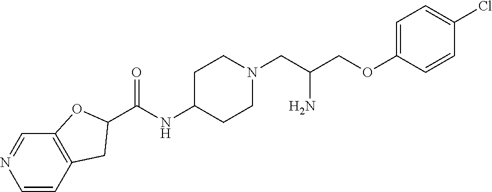 [SMILES: NC(COc1ccc(Cl)cc1)CN1CCC(NC(=O)C2Cc3ccncc3O2)CC1] |
| X-106 | 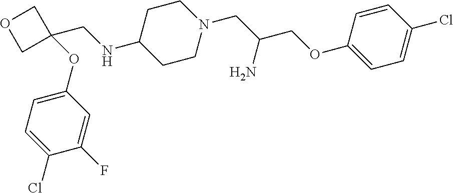 [SMILES: NC(COc1ccc(Cl)cc1)CN1CCC(NCC2(Oc3ccc(Cl)c(F)c3)COC2)CC1] |
| X-107 | 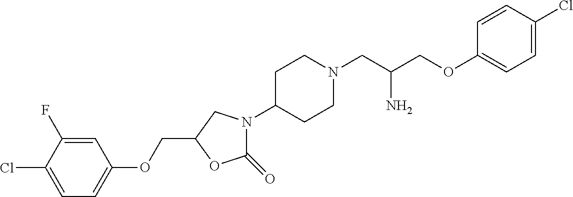 [SMILES: NC(COc1ccc(Cl)cc1)CN1CCC(N2CC(COc3ccc(Cl)c(F)c3)OC2=O)CC1] |
| X-108 | 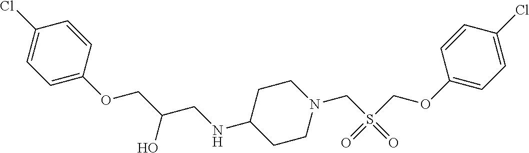 [SMILES: O=S(=O)(COc1ccc(Cl)cc1)CN1CCC(NCC(O)COc2ccc(Cl)cc2)CC1] |
| X-109 | 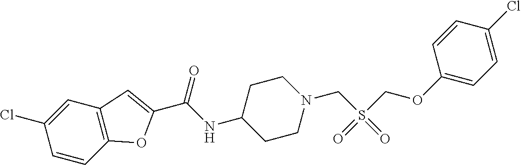 [SMILES: O=C(NC1CCN(CS(=O)(=O)COc2ccc(Cl)cc2)CC1)c1cc2cc(Cl)ccc2o1] |
| X-110 | 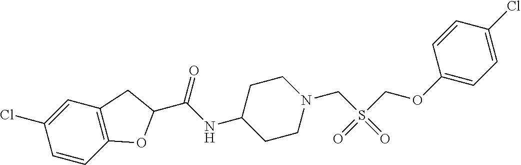 [SMILES: O=C(NC1CCN(CS(=O)(=O)COc2ccc(Cl)cc2)CC1)C1Cc2cc(Cl)ccc2O1] |
| X-111 | 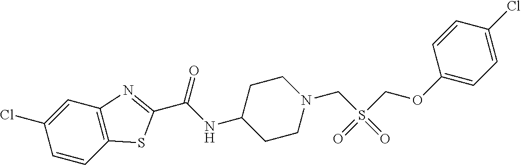 [SMILES: O=C(NC1CCN(CS(=O)(=O)COc2ccc(Cl)cc2)CC1)c1nc2cc(Cl)ccc2s1] |
| X-112 | 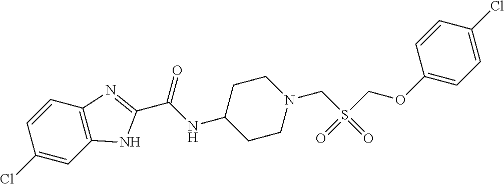 [SMILES: O=C(NC1CCN(CS(=O)(=O)COc2ccc(Cl)cc2)CC1)c1nc2ccc(Cl)cc2[nH]1] |
| X-113 | 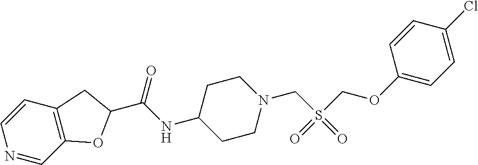 [SMILES: O=C(NC1CCN(CS(=O)(=O)COc2ccc(Cl)cc2)CC1)C1Cc2ccncc2O1] |
| X-114 | 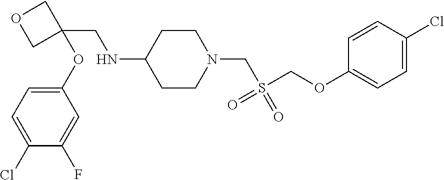 [SMILES: O=S(=O)(COc1ccc(Cl)cc1)CN1CCC(NCC2(Oc3ccc(Cl)c(F)c3)COC2)CC1] |
| X-115 | 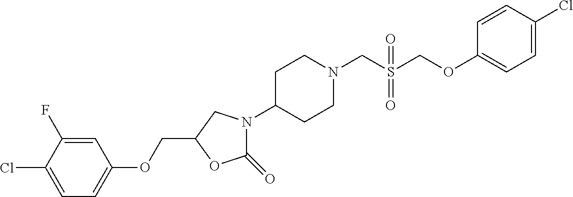 [SMILES: O=C1OC(COc2ccc(Cl)c(F)c2)CN1C1CCN(CS(=O)(=O)COc2ccc(Cl)cc2)CC1] |
| X-116 | 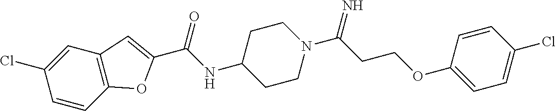 [SMILES: N=C(CCOc1ccc(Cl)cc1)N1CCC(NC(=O)c2cc3cc(Cl)ccc3o2)CC1] |
| X-117 | 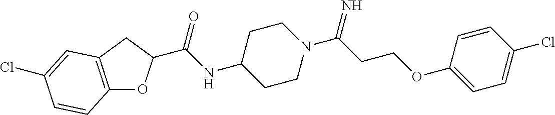 [SMILES: N=C(CCOc1ccc(Cl)cc1)N1CCC(NC(=O)C2Cc3cc(Cl)ccc3O2)CC1] |
| X-118 | 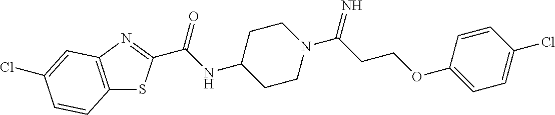 [SMILES: N=C(CCOc1ccc(Cl)cc1)N1CCC(NC(=O)c2nc3cc(Cl)ccc3s2)CC1] |
| X-119 | 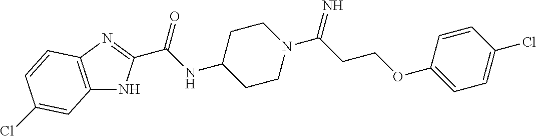 [SMILES: N=C(CCOc1ccc(Cl)cc1)N1CCC(NC(=O)c2nc3ccc(Cl)cc3[nH]2)CC1] |
| X-120 | 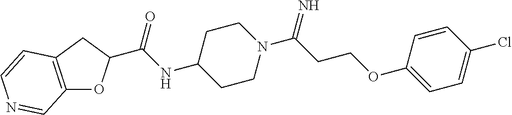 [SMILES: N=C(CCOc1ccc(Cl)cc1)N1CCC(NC(=O)C2Cc3ccncc3O2)CC1] |
| X-121 | 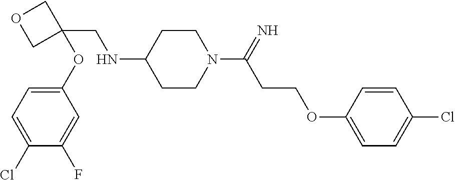 [SMILES: N=C(CCOc1ccc(Cl)cc1)N1CCC(NCC2(Oc3ccc(Cl)c(F)c3)COC2)CC1] |
| X-122 | 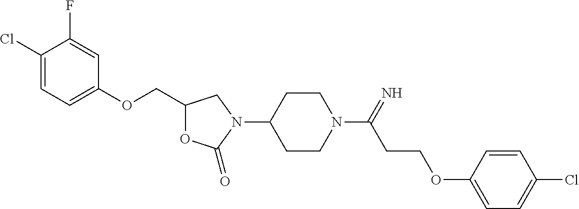 [SMILES: N=C(CCOc1ccc(Cl)cc1)N1CCC(N2CC(COc3ccc(Cl)c(F)c3)OC2=O)CC1] |
| X-123 | 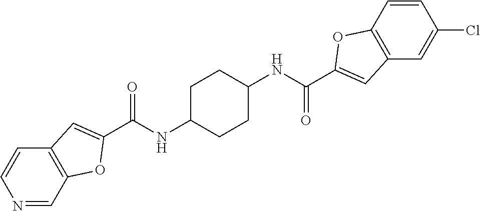 [SMILES: O=C(NC1CCC(NC(=O)c2cc3ccncc3o2)CC1)c1cc2cc(Cl)ccc2o1] |
| X-124 | 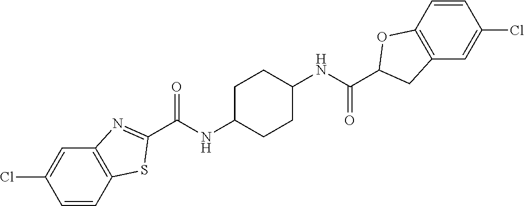 [SMILES: O=C(NC1CCC(NC(=O)C2Cc3cc(Cl)ccc3O2)CC1)c1nc2cc(Cl)ccc2s1] |
| X-125 | 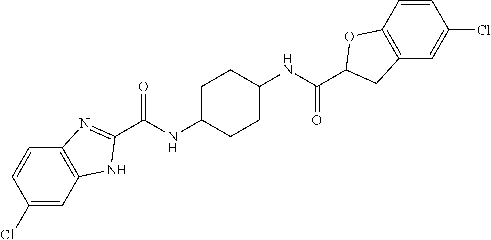 [SMILES: O=C(NC1CCC(NC(=O)C2Cc3cc(Cl)ccc3O2)CC1)c1nc2ccc(Cl)cc2[nH]1] |
| X-126 | 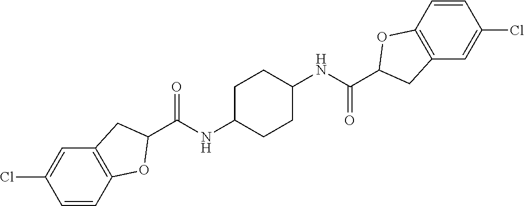 [SMILES: O=C(NC1CCC(NC(=O)C2Cc3cc(Cl)ccc3O2)CC1)C1Cc2cc(Cl)ccc2O1] |
| X-127 | 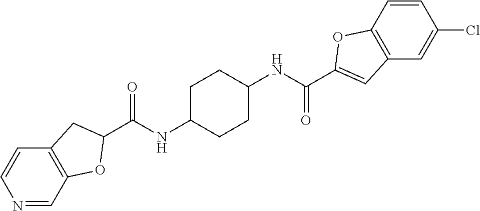 [SMILES: O=C(NC1CCC(NC(=O)C2Cc3ccncc3O2)CC1)c1cc2cc(Cl)ccc2o1] |
| X-128 | 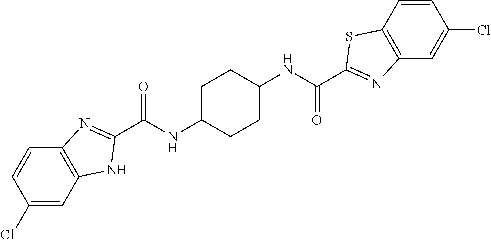 [SMILES: O=C(NC1CCC(NC(=O)c2nc3cc(Cl)ccc3s2)CC1)c1nc2ccc(Cl)cc2[nH]1] |
| X-129 | 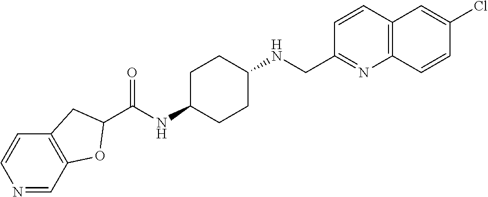 [SMILES: O=C(N[C@H]1CC[C@H](NCc2ccc3cc(Cl)ccc3n2)CC1)C1Cc2ccncc2O1] |
| X-130 | 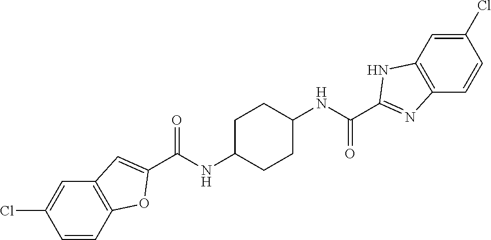 [SMILES: O=C(NC1CCC(NC(=O)c2cc3cc(Cl)ccc3o2)CC1)c1nc2ccc(Cl)cc2[nH]1] |
| X-131 | 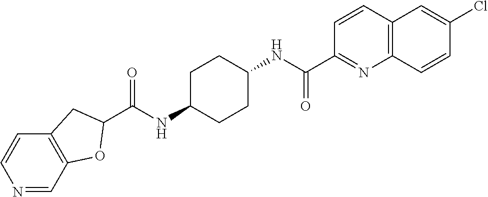 [SMILES: O=C(N[C@H]1CC[C@H](NC(=O)C2Cc3ccncc3O2)CC1)c1ccc2cc(Cl)ccc2n1] |
| X-132 | 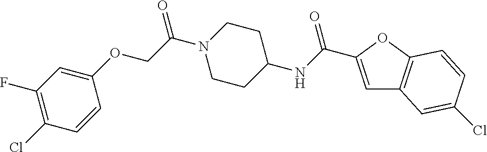 [SMILES: O=C(NC1CCN(C(=O)COc2ccc(Cl)c(F)c2)CC1)c1cc2cc(Cl)ccc2o1] |
| X-133 | 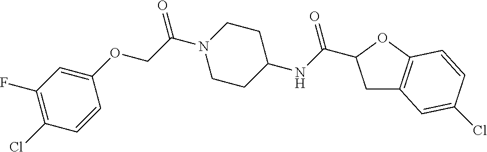 [SMILES: O=C(NC1CCN(C(=O)COc2ccc(Cl)c(F)c2)CC1)C1Cc2cc(Cl)ccc2O1] |
| X-134 | 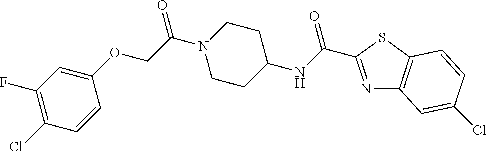 [SMILES: O=C(NC1CCN(C(=O)COc2ccc(Cl)c(F)c2)CC1)c1nc2cc(Cl)ccc2s1] |
| X-135 | 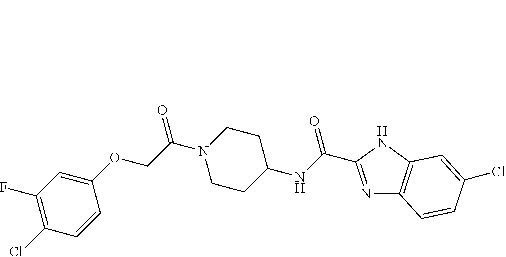 [SMILES: O=C(NC1CCN(C(=O)COc2ccc(Cl)c(F)c2)CC1)c1nc2ccc(Cl)cc2[nH]1] |
| X-136 | 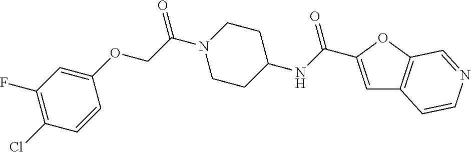 [SMILES: O=C(NC1CCN(C(=O)COc2ccc(Cl)c(F)c2)CC1)c1cc2ccncc2o1] |
| X-137 | 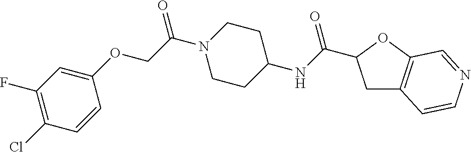 [SMILES: O=C(NC1CCN(C(=O)COc2ccc(Cl)c(F)c2)CC1)C1Cc2ccncc2O1] |
| X-138 | 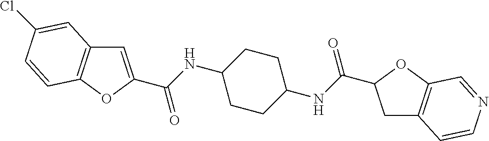 [SMILES: O=C(NC1CCC(NC(=O)C2Cc3ccncc3O2)CC1)c1cc2cc(Cl)ccc2o1] |
| X-139 | 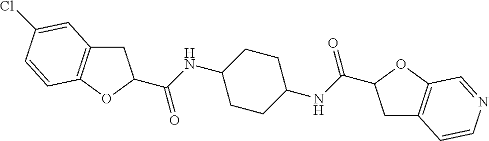 [SMILES: O=C(NC1CCC(NC(=O)C2Cc3ccncc3O2)CC1)C1Cc2cc(Cl)ccc2O1] |
| X-140 | 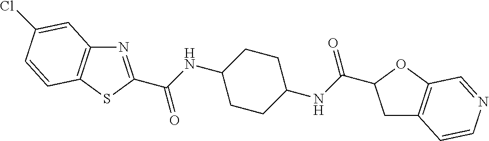 [SMILES: O=C(NC1CCC(NC(=O)C2Cc3ccncc3O2)CC1)c1nc2cc(Cl)ccc2s1] |
| X-141 | 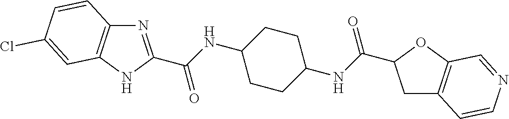 [SMILES: O=C(NC1CCC(NC(=O)C2Cc3ccncc3O2)CC1)c1nc2ccc(Cl)cc2[nH]1] |
| X-142 | 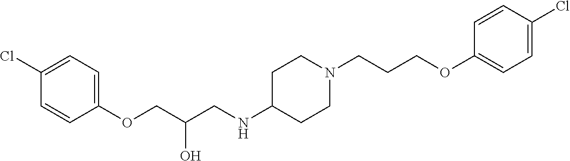 [SMILES: OC(CNC1CCN(CCCOc2ccc(Cl)cc2)CC1)COc1ccc(Cl)cc1] |
| X-143 | 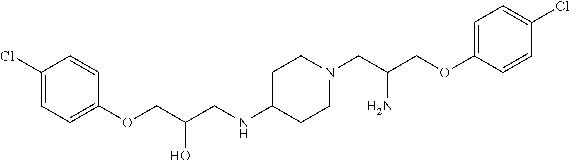 [SMILES: NC(COc1ccc(Cl)cc1)CN1CCC(NCC(O)COc2ccc(Cl)cc2)CC1] |
| X-144 | 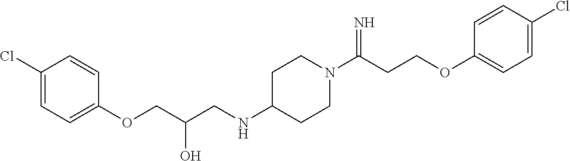 [SMILES: N=C(CCOc1ccc(Cl)cc1)N1CCC(NCC(O)COc2ccc(Cl)cc2)CC1] |
| X-145 | 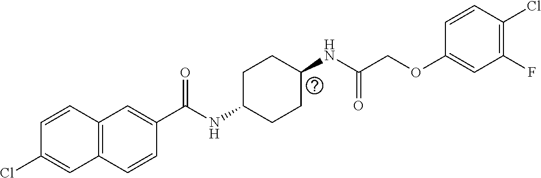 [SMILES: O=C(COc1ccc(Cl)c(F)c1)N[C@H]1CC[C@H](NC(=O)c2ccc3cc(Cl)ccc3c2)CC1] |
| X-146 | 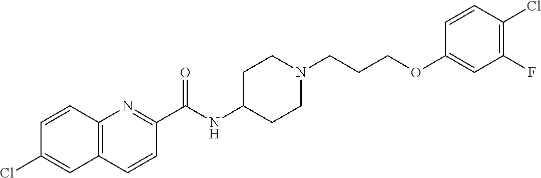 [SMILES: O=C(NC1CCN(CCCOc2ccc(Cl)c(F)c2)CC1)c1ccc2cc(Cl)ccc2n1] |
| X-147 | 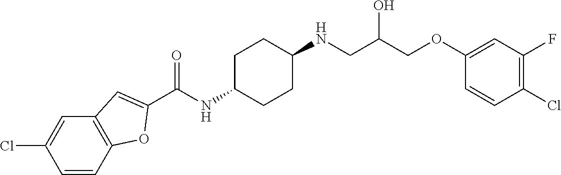 [SMILES: O=C(N[C@H]1CC[C@H](NCC(O)COc2ccc(Cl)c(F)c2)CC1)c1cc2cc(Cl)ccc2o1] |
| X-148 | 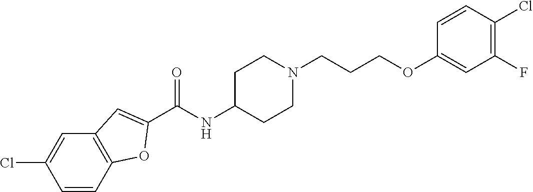 [SMILES: O=C(NC1CCN(CCCOc2ccc(Cl)c(F)c2)CC1)c1cc2cc(Cl)ccc2o1] |
| X-149 | 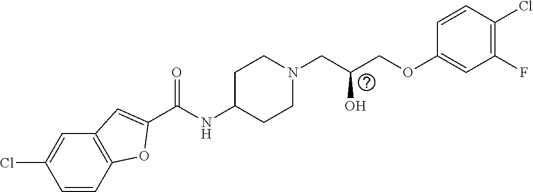 [SMILES: O=C(NC1CCN(C[C@H](O)COc2ccc(Cl)c(F)c2)CC1)c1cc2cc(Cl)ccc2o1] |
| X-150 | 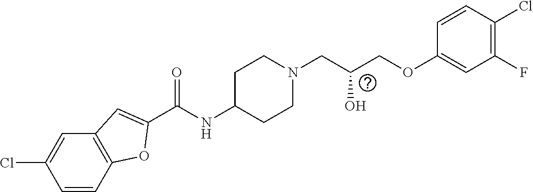 [SMILES: O=C(NC1CCN(C[C@@H](O)COc2ccc(Cl)c(F)c2)CC1)c1cc2cc(Cl)ccc2o1] |
| X-151 | 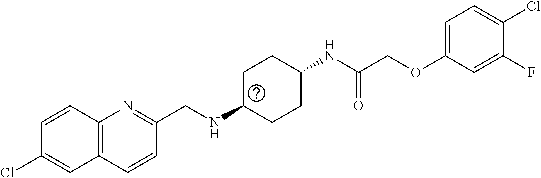 [SMILES: O=C(COc1ccc(Cl)c(F)c1)N[C@H]1CC[C@H](NCc2ccc3cc(Cl)ccc3n2)CC1] |
| X-152 | 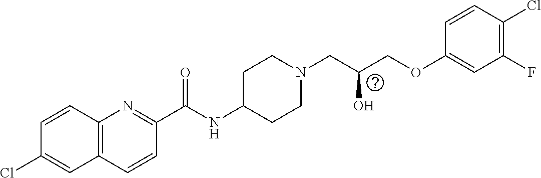 [SMILES: O=C(NC1CCN(C[C@H](O)COc2ccc(Cl)c(F)c2)CC1)c1ccc2cc(Cl)ccc2n1] |
| X-153 | 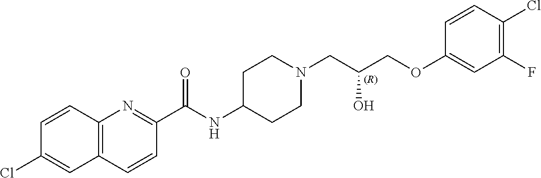 |
| X-154 | 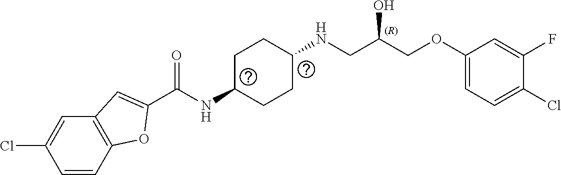 |
| X-155 | 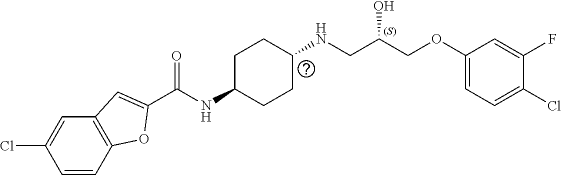 |
| X-156 | 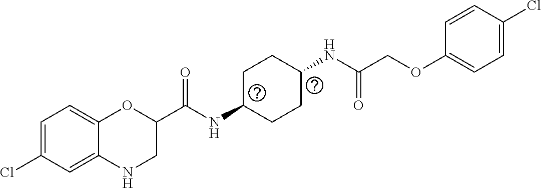 [SMILES: O=C(COc1ccc(Cl)cc1)N[C@H]1CC[C@H](NC(=O)C2CNc3cc(Cl)ccc3O2)CC1] |
| X-157 | 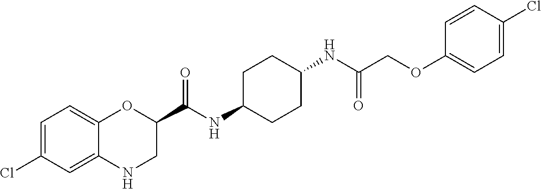 [SMILES: O=C(COc1ccc(Cl)cc1)N[C@H]1CC[C@H](NC(=O)[C@H]2CNc3cc(Cl)ccc3O2)CC1] |
| X-158 | 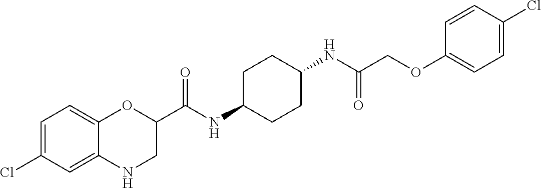 [SMILES: O=C(COc1ccc(Cl)cc1)N[C@H]1CC[C@H](NC(=O)C2CNc3cc(Cl)ccc3O2)CC1] |
| X-159 | 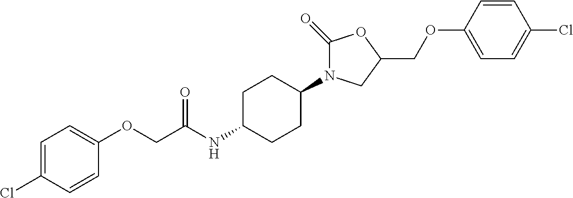 [SMILES: O=C(COc1ccc(Cl)cc1)N[C@H]1CC[C@H](N2CC(COc3ccc(Cl)cc3)OC2=O)CC1] |
| X-160 | 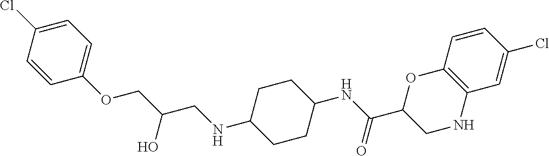 [SMILES: O=C(NC1CCC(NCC(O)COc2ccc(Cl)cc2)CC1)C1CNc2cc(Cl)ccc2O1] |
| X-161 | 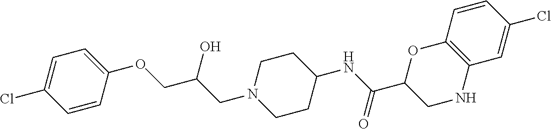 [SMILES: O=C(NC1CCN(CC(O)COc2ccc(Cl)cc2)CC1)C1CNc2cc(Cl)ccc2O1] |
| X-162 | 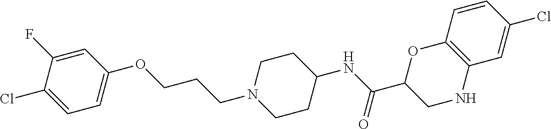 [SMILES: O=C(NC1CCN(CCCOc2ccc(Cl)c(F)c2)CC1)C1CNc2cc(Cl)ccc2O1] |
| X-163 | 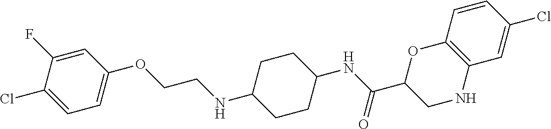 [SMILES: O=C(NC1CCC(NCCOc2ccc(Cl)c(F)c2)CC1)C1CNc2cc(Cl)ccc2O1] |
| X-164 | 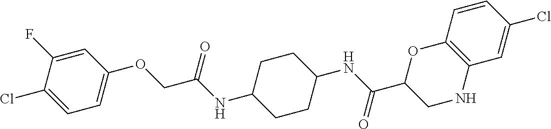 [SMILES: O=C(COc1ccc(Cl)c(F)c1)NC1CCC(NC(=O)C2CNc3cc(Cl)ccc3O2)CC1] |
| X-165 | 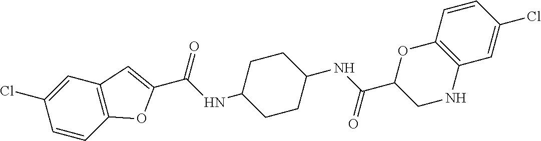 [SMILES: O=C(NC1CCC(NC(=O)C2CNc3cc(Cl)ccc3O2)CC1)c1cc2cc(Cl)ccc2o1] |
| X-166 | 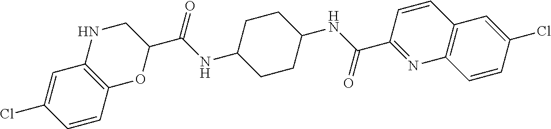 [SMILES: O=C(NC1CCC(NC(=O)C2CNc3cc(Cl)ccc3O2)CC1)c1ccc2cc(Cl)ccc2n1] |

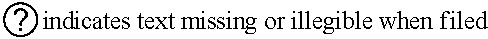

TABLE 1XX

| Cpd | |
|---|---|
| No. | Structure |
| XX-1 | 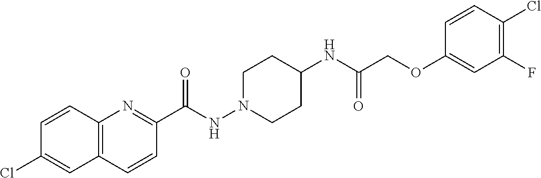 [SMILES: O=C(COc1ccc(Cl)c(F)c1)NC1CCN(NC(=O)c2ccc3cc(Cl)ccc3n2)CC1] |
| XX-2 | 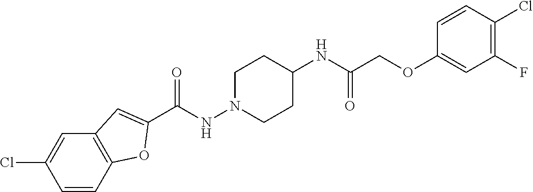 [SMILES: O=C(COc1ccc(Cl)c(F)c1)NC1CCN(NC(=O)c2cc3cc(Cl)ccc3o2)CC1] |
| XX-3 | 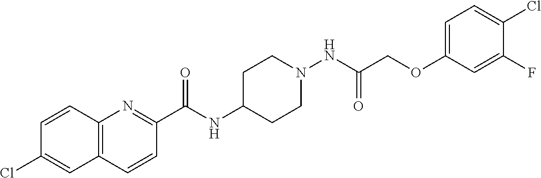 [SMILES: O=C(COc1ccc(Cl)c(F)c1)NN1CCC(NC(=O)c2ccc3cc(Cl)ccc3n2)CC1] |
| XX-4 | 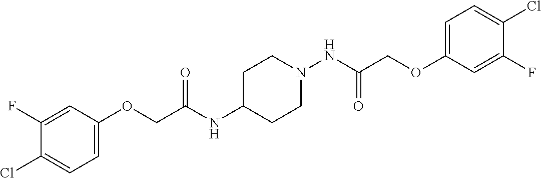 [SMILES: O=C(COc1ccc(Cl)c(F)c1)NC1CCN(NC(=O)COc2ccc(Cl)c(F)c2)CC1] |
| XX-5 | 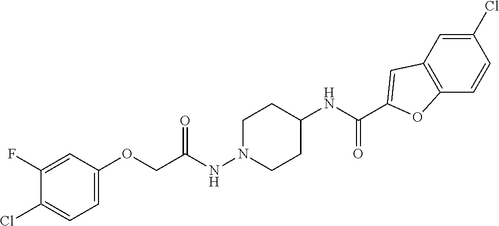 [SMILES: O=C(COc1ccc(Cl)c(F)c1)NN1CCC(NC(=O)c2cc3cc(Cl)ccc3o2)CC1] |
| XX-6 | 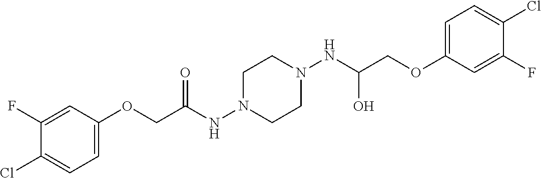 [SMILES: O=C(COc1ccc(Cl)c(F)c1)NN1CCN(NC(O)COc2ccc(Cl)c(F)c2)CC1] |
| XX-7 | 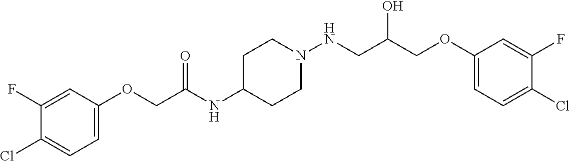 [SMILES: O=C(COc1ccc(Cl)c(F)c1)NC1CCN(NCC(O)COc2ccc(Cl)c(F)c2)CC1] |
| XX-8 | 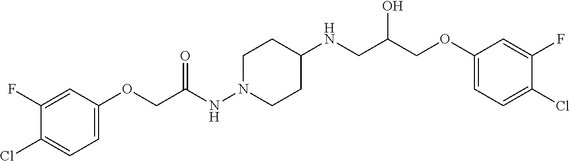 [SMILES: O=C(COc1ccc(Cl)c(F)c1)NN1CCC(NCC(O)COc2ccc(Cl)c(F)c2)CC1] |
| XX-9 | 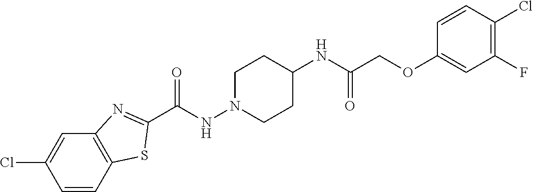 [SMILES: O=C(COc1ccc(Cl)c(F)c1)NC1CCN(NC(=O)c2nc3cc(Cl)ccc3s2)CC1] |
| XX-10 | 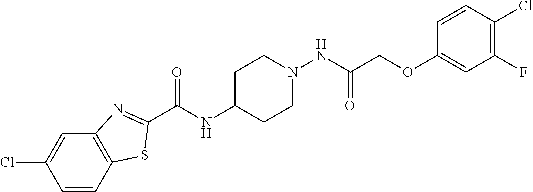 [SMILES: O=C(COc1ccc(Cl)c(F)c1)NN1CCC(NC(=O)c2nc3cc(Cl)ccc3s2)CC1] |
| XX-11 | 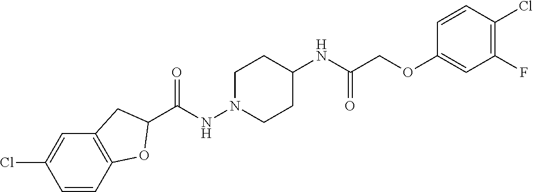 [SMILES: O=C(COc1ccc(Cl)c(F)c1)NC1CCN(NC(=O)C2Cc3cc(Cl)ccc3O2)CC1] |
| XX-12 | 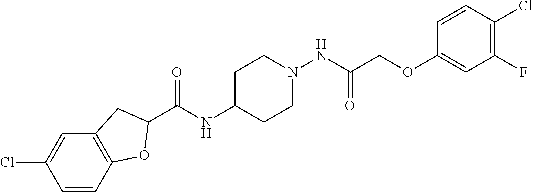 [SMILES: O=C(COc1ccc(Cl)c(F)c1)NN1CCC(NC(=O)C2Cc3cc(Cl)ccc3O2)CC1] |
| XX-13 | 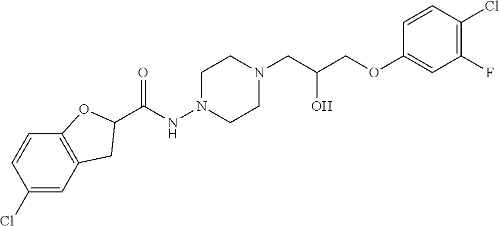 [SMILES: O=C(NN1CCN(CC(O)COc2ccc(Cl)c(F)c2)CC1)C1Cc2cc(Cl)ccc2O1] |
| XX-14 | 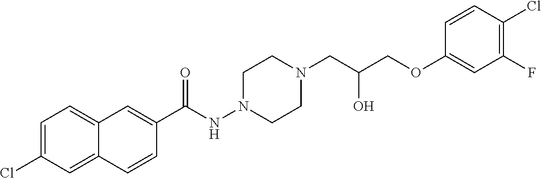 [SMILES: O=C(NN1CCN(CC(O)COc2ccc(Cl)c(F)c2)CC1)c1ccc2cc(Cl)ccc2c1] |
| XX-15 | 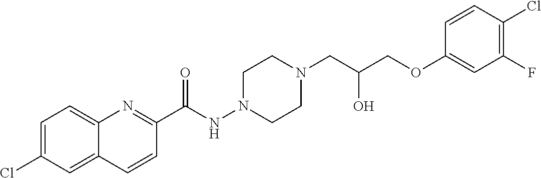 [SMILES: O=C(NN1CCN(CC(O)COc2ccc(Cl)c(F)c2)CC1)c1ccc2cc(Cl)ccc2n1] |
| XX-16 | 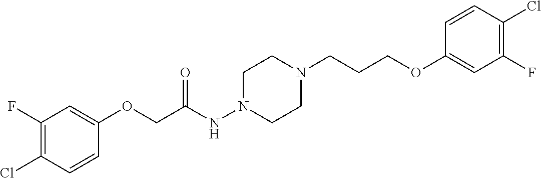 [SMILES: O=C(COc1ccc(Cl)c(F)c1)NN1CCN(CCCOc2ccc(Cl)c(F)c2)CC1] |
| XX-17 | 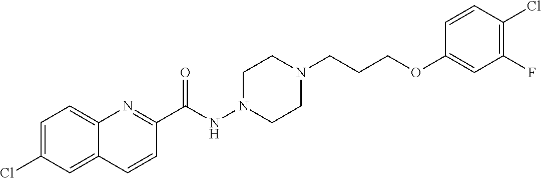 [SMILES: O=C(NN1CCN(CCCOc2ccc(Cl)c(F)c2)CC1)c1ccc2cc(Cl)ccc2n1] |
| XX-18 | 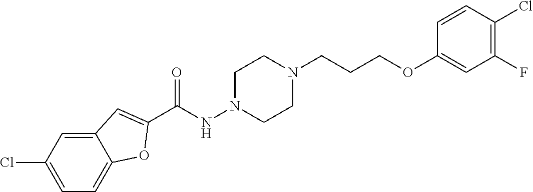 [SMILES: O=C(NN1CCN(CCCOc2ccc(Cl)c(F)c2)CC1)c1cc2cc(Cl)ccc2o1] |
| XX-19 | 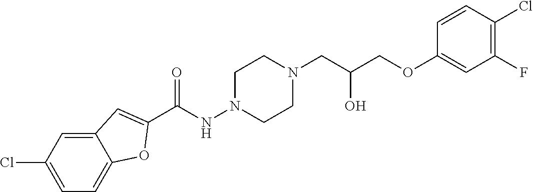 [SMILES: O=C(NN1CCN(CC(O)COc2ccc(Cl)c(F)c2)CC1)c1cc2cc(Cl)ccc2o1] |
| XX-20 | 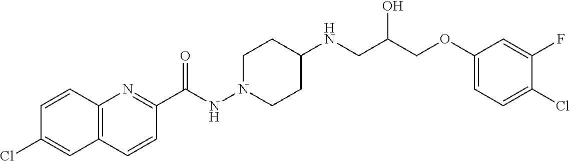 [SMILES: O=C(NN1CCC(NCC(O)COc2ccc(Cl)c(F)c2)CC1)c1ccc2cc(Cl)ccc2n1] |
| XX-21 | 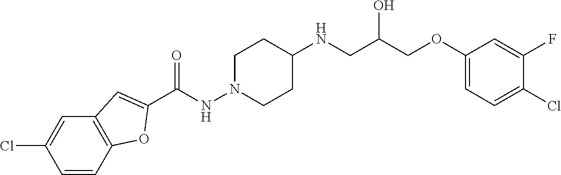 [SMILES: O=C(NN1CCC(NCC(O)COc2ccc(Cl)c(F)c2)CC1)c1cc2cc(Cl)ccc2o1] |
| XX-22 | 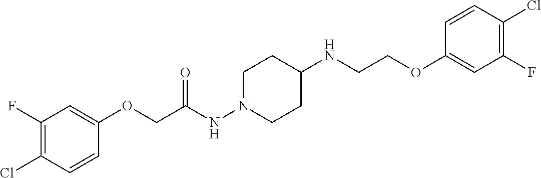 [SMILES: O=C(COc1ccc(Cl)c(F)c1)NN1CCC(NCCOc2ccc(Cl)c(F)c2)CC1] |
| XX-23 | 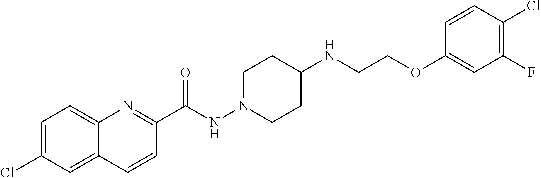 [SMILES: O=C(NN1CCC(NCCOc2ccc(Cl)c(F)c2)CC1)c1ccc2cc(Cl)ccc2n1] |
| XX-24 | 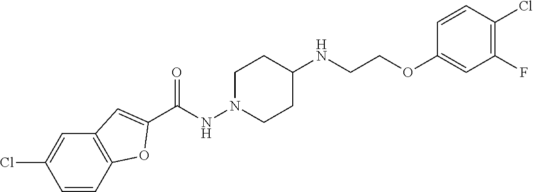 [SMILES: O=C(NN1CCC(NCCOc2ccc(Cl)c(F)c2)CC1)c1cc2cc(Cl)ccc2o1] |
| XX-25 | 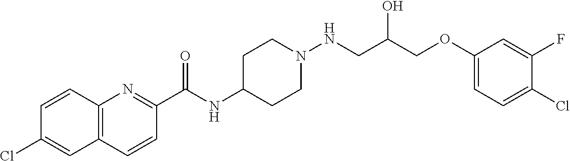 [SMILES: O=C(NC1CCN(NCC(O)COc2ccc(Cl)c(F)c2)CC1)c1ccc2cc(Cl)ccc2n1] |
| XX-26 | 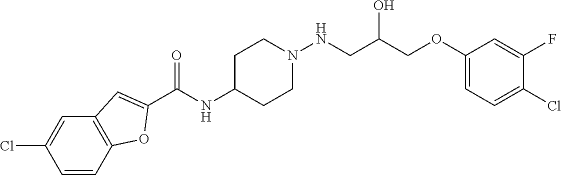 [SMILES: O=C(NC1CCN(NCC(O)COc2ccc(Cl)c(F)c2)CC1)c1cc2cc(Cl)ccc2o1] |
| XX-27 | 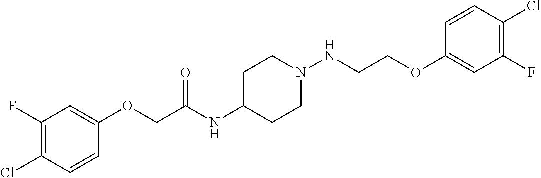 [SMILES: O=C(COc1ccc(Cl)c(F)c1)NC1CCN(NCCOc2ccc(Cl)c(F)c2)CC1] |
| XX-28 | 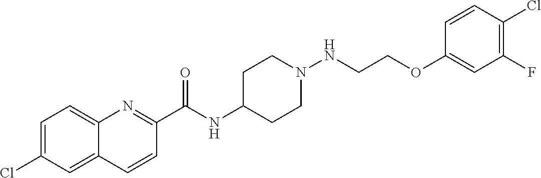 [SMILES: O=C(NC1CCN(NCCOc2ccc(Cl)c(F)c2)CC1)c1ccc2cc(Cl)ccc2n1] |
| XX-29 | 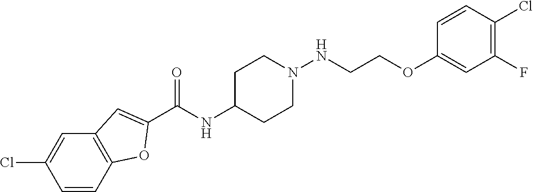 [SMILES: O=C(NC1CCN(NCCOc2ccc(Cl)c(F)c2)CC1)c1cc2cc(Cl)ccc2o1] |
| XX-30 | 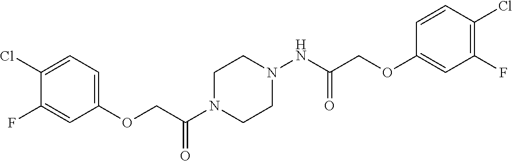 [SMILES: O=C(COc1ccc(Cl)c(F)c1)NN1CCN(C(=O)COc2ccc(Cl)c(F)c2)CC1] |
| XX-31 | 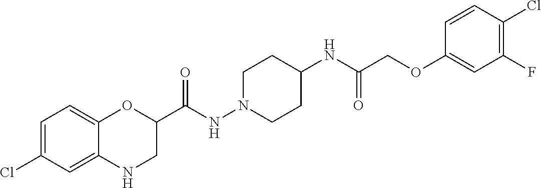 [SMILES: O=C(COc1ccc(Cl)c(F)c1)NC1CCN(NC(=O)C2CNc3cc(Cl)ccc3O2)CC1] |
| XX-32 | 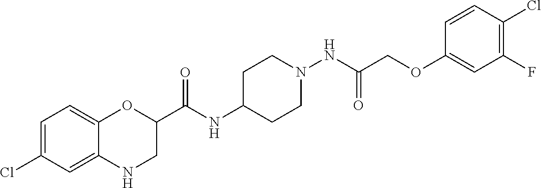 [SMILES: O=C(COc1ccc(Cl)c(F)c1)NN1CCC(NC(=O)C2CNc3cc(Cl)ccc3O2)CC1] |
| XX-33 | 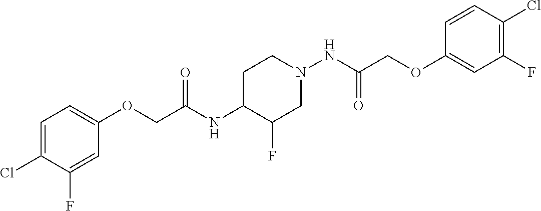 [SMILES: O=C(COc1ccc(Cl)c(F)c1)NC1CCN(NC(=O)COc2ccc(Cl)c(F)c2)CC1F] |
| XX-34 | 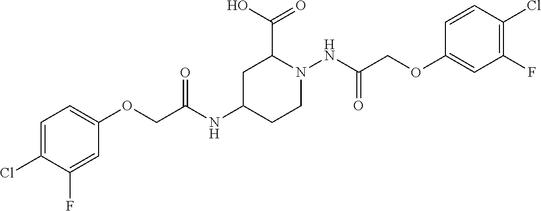 [SMILES: O=C(COc1ccc(Cl)c(F)c1)NC1CCN(NC(=O)COc2ccc(Cl)c(F)c2)C(C(=O)O)C1] |
| XX-35 | 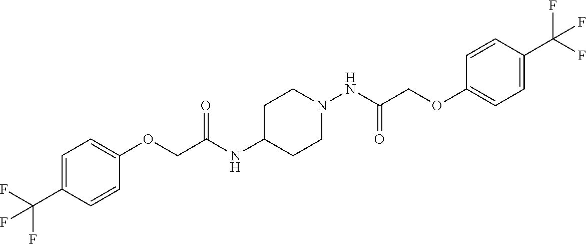 [SMILES: O=C(COc1ccc(C(F)(F)F)cc1)NC1CCN(NC(=O)COc2ccc(C(F)(F)F)cc2)CC1] |
| XX-36 | 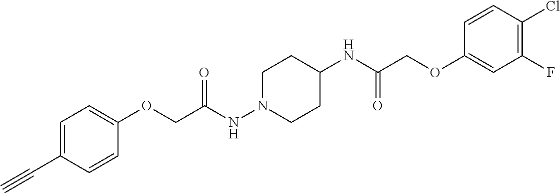 [SMILES: C#Cc1ccc(OCC(=O)NN2CCC(NC(=O)COc3ccc(Cl)c(F)c3)CC2)cc1] |
| XX-37 | 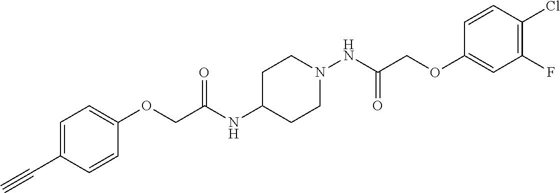 [SMILES: C#Cc1ccc(OCC(=O)NC2CCN(NC(=O)COc3ccc(Cl)c(F)c3)CC2)cc1] |
| XX-38 | 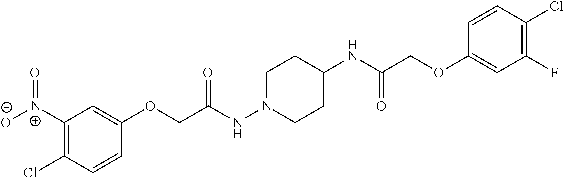 [SMILES: O=C(COc1ccc(Cl)c(F)c1)NC1CCN(NC(=O)COc2ccc(Cl)c([N+](=O)[O-])c2)CC1] |
| XX-39 | 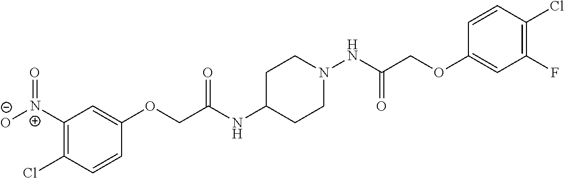 [SMILES: O=C(COc1ccc(Cl)c([N+](=O)[O-])c1)NC1CCN(NC(=O)COc2ccc(Cl)c(F)c2)CC1] |
| XX-40 | 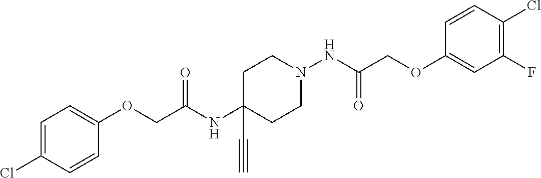 [SMILES: C#CC1(NC(=O)COc2ccc(Cl)cc2)CCN(NC(=O)COc2ccc(Cl)c(F)c2)CC1] |
| XX-41 | 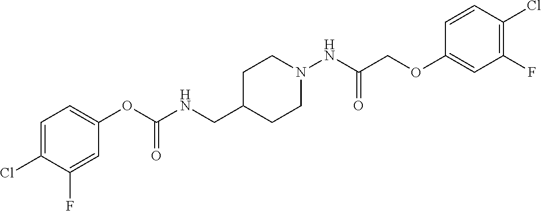 [SMILES: O=C(COc1ccc(Cl)c(F)c1)NN1CCC(CNC(=O)Oc2ccc(Cl)c(F)c2)CC1] |
| XX-42 | 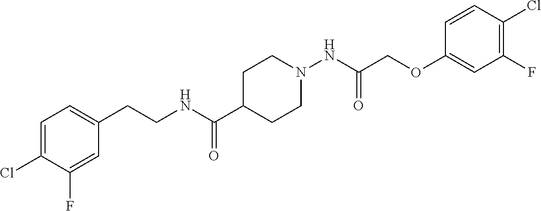 [SMILES: O=C(COc1ccc(Cl)c(F)c1)NN1CCC(C(=O)NCCc2ccc(Cl)c(F)c2)CC1] |
| XX-43 | 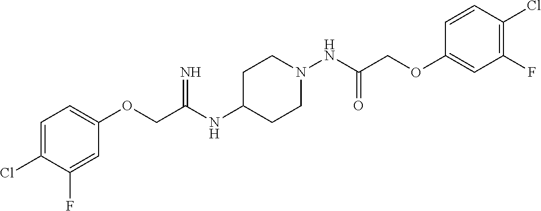 [SMILES: N=C(COc1ccc(Cl)c(F)c1)NC1CCN(NC(=O)COc2ccc(Cl)c(F)c2)CC1] |
| XX-44 | 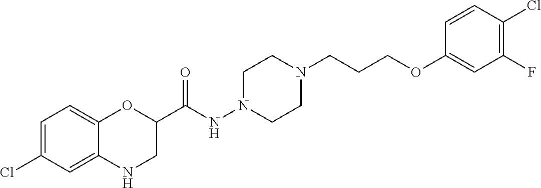 [SMILES: O=C(NN1CCN(CCCOc2ccc(Cl)c(F)c2)CC1)C1CNc2cc(Cl)ccc2O1] |
| XX-45 | 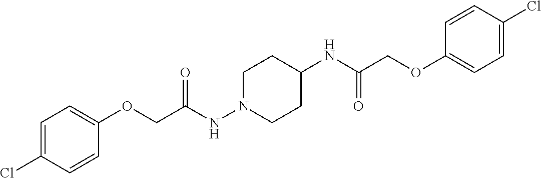 [SMILES: O=C(COc1ccc(Cl)cc1)NC1CCN(NC(=O)COc2ccc(Cl)cc2)CC1] |
| XX-46 | 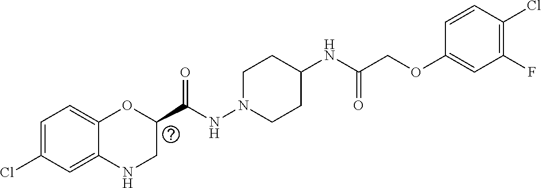 [SMILES: O=C(COc1ccc(Cl)c(F)c1)NC1CCN(NC(=O)[C@H]2CNc3cc(Cl)ccc3O2)CC1] |
| XX-47 | 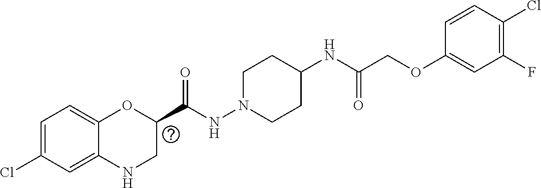 [SMILES: O=C(COc1ccc(Cl)c(F)c1)NC1CCN(NC(=O)[C@H]2CNc3cc(Cl)ccc3O2)CC1] |
| XX-48 | 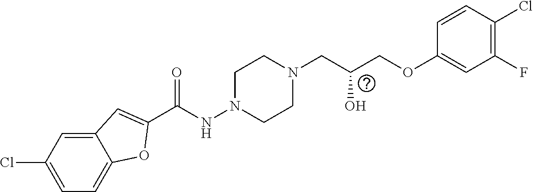 [SMILES: O=C(NN1CCN(C[C@@H](O)COc2ccc(Cl)c(F)c2)CC1)c1cc2cc(Cl)ccc2o1] |
| XX-49 | 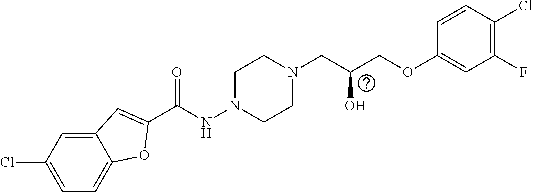 [SMILES: O=C(NN1CCN(C[C@H](O)COc2ccc(Cl)c(F)c2)CC1)c1cc2cc(Cl)ccc2o1] |
| XX-50 | 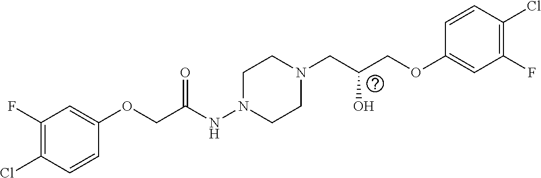 [SMILES: O=C(COc1ccc(Cl)c(F)c1)NN1CCN(C[C@@H](O)COc2ccc(Cl)c(F)c2)CC1] |
| XX-51 | 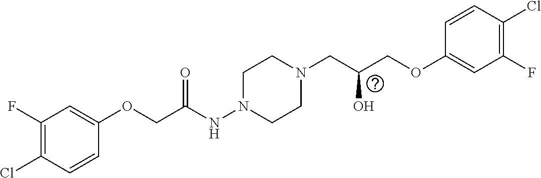 [SMILES: O=C(COc1ccc(Cl)c(F)c1)NN1CCN(C[C@H](O)COc2ccc(Cl)c(F)c2)CC1] |
| XX-52 | 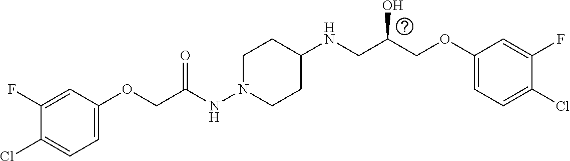 [SMILES: O=C(COc1ccc(Cl)c(F)c1)NN1CCC(NC[C@@H](O)COc2ccc(Cl)c(F)c2)CC1] |
| XX-53 | 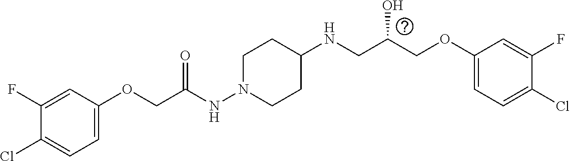 [SMILES: O=C(COc1ccc(Cl)c(F)c1)NN1CCC(NC[C@H](O)COc2ccc(Cl)c(F)c2)CC1] |
| XX-54 | 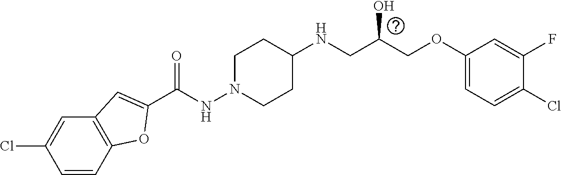 [SMILES: O=C(NN1CCC(NC[C@@H](O)COc2ccc(Cl)c(F)c2)CC1)c1cc2cc(Cl)ccc2o1] |
| XX-55 | 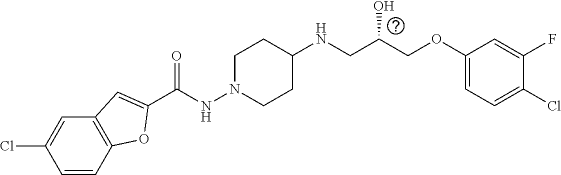 [SMILES: O=C(NN1CCC(NC[C@H](O)COc2ccc(Cl)c(F)c2)CC1)c1cc2cc(Cl)ccc2o1] |
| XX-56 | 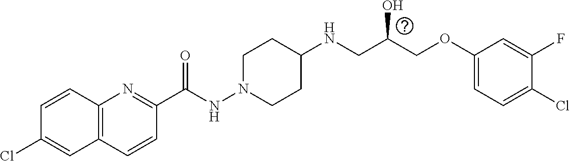 [SMILES: O=C(NN1CCC(NC[C@@H](O)COc2ccc(Cl)c(F)c2)CC1)c1ccc2cc(Cl)ccc2n1] |
| XX-57 | 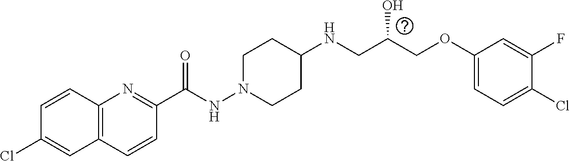 [SMILES: O=C(NN1CCC(NC[C@H](O)COc2ccc(Cl)c(F)c2)CC1)c1ccc2cc(Cl)ccc2n1] |
| XX-58 | 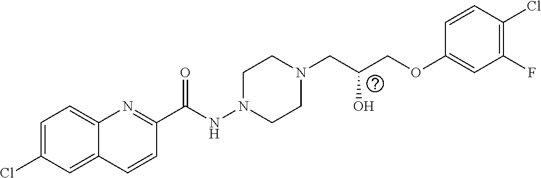 [SMILES: O=C(NN1CCN(C[C@@H](O)COc2ccc(Cl)c(F)c2)CC1)c1ccc2cc(Cl)ccc2n1] |
| XX-59 | 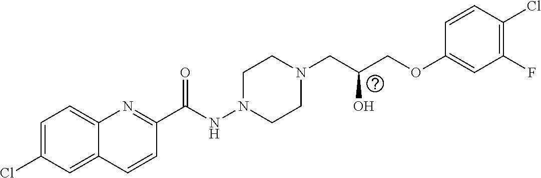 [SMILES: O=C(NN1CCN(C[C@H](O)COc2ccc(Cl)c(F)c2)CC1)c1ccc2cc(Cl)ccc2n1] |
| XX-60 | 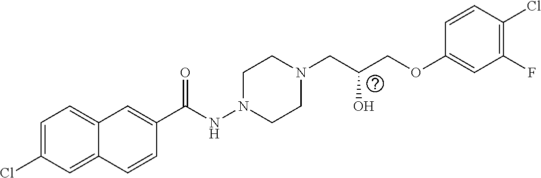 [SMILES: O=C(NN1CCN(C[C@@H](O)COc2ccc(Cl)c(F)c2)CC1)c1ccc2cc(Cl)ccc2c1] |
| XX-61 | 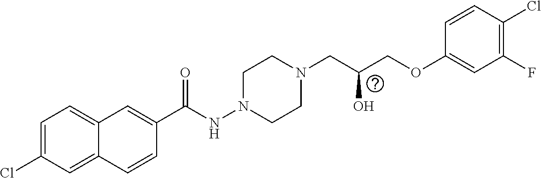 [SMILES: O=C(NN1CCN(C[C@H](O)COc2ccc(Cl)c(F)c2)CC1)c1ccc2cc(Cl)ccc2c1] |
| XX-62 | 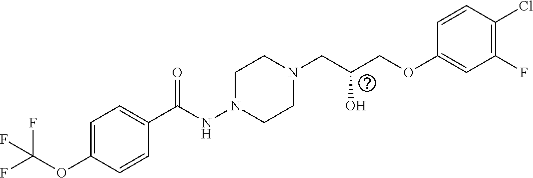 [SMILES: O=C(NN1CCN(C[C@@H](O)COc2ccc(Cl)c(F)c2)CC1)c1ccc(OC(F)(F)F)cc1] |
| XX-63 | 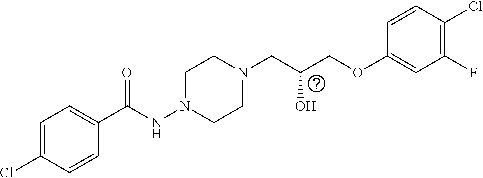 [SMILES: O=C(NN1CCN(C[C@@H](O)COc2ccc(Cl)c(F)c2)CC1)c1ccc(Cl)cc1] |
| XX-64 | 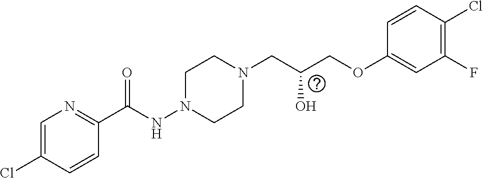 [SMILES: O=C(NN1CCN(C[C@@H](O)COc2ccc(Cl)c(F)c2)CC1)c1ccc(Cl)cn1] |
| XX-65 | 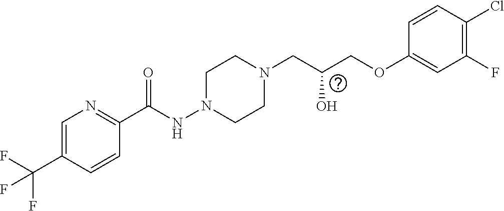 [SMILES: O=C(NN1CCN(C[C@@H](O)COc2ccc(Cl)c(F)c2)CC1)c1ccc(C(F)(F)F)cn1] |
| XX-66 | 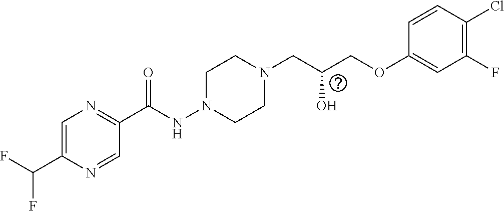 [SMILES: O=C(NN1CCN(C[C@@H](O)COc2ccc(Cl)c(F)c2)CC1)c1cnc(C(F)F)cn1] |
| XX-67 | 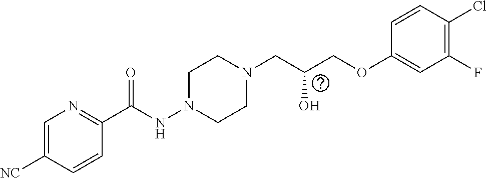 [SMILES: N#Cc1ccc(C(=O)NN2CCN(C[C@@H](O)COc3ccc(Cl)c(F)c3)CC2)nc1] |
| XX-68 | 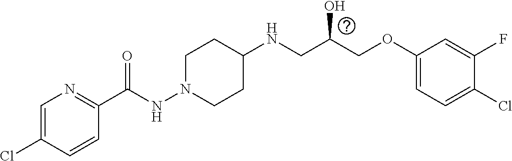 [SMILES: O=C(NN1CCC(NC[C@@H](O)COc2ccc(Cl)c(F)c2)CC1)c1ccc(Cl)cn1] |
| XX-69 | 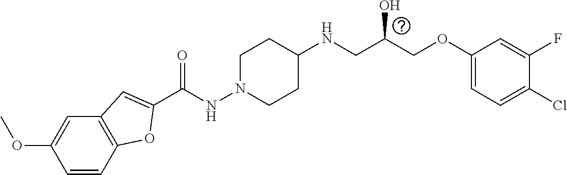 [SMILES: COc1ccc2oc(C(=O)NN3CCC(NC[C@@H](O)COc4ccc(Cl)c(F)c4)CC3)cc2c1] |
| XX-70 | 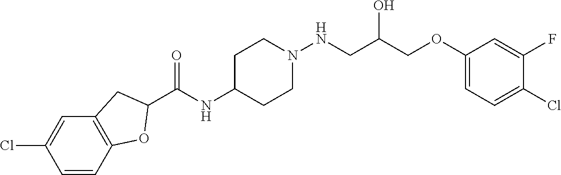 [SMILES: O=C(NC1CCN(NCC(O)COc2ccc(Cl)c(F)c2)CC1)C1Cc2cc(Cl)ccc2O1] |
| XX-71 | 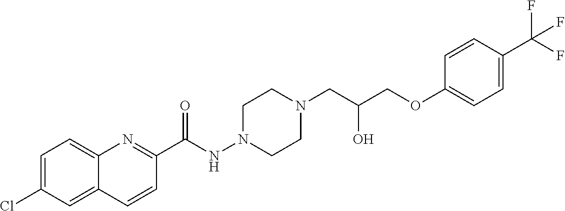 [SMILES: O=C(NN1CCN(CC(O)COc2ccc(C(F)(F)F)cc2)CC1)c1ccc2cc(Cl)ccc2n1] |
| XX-72 | 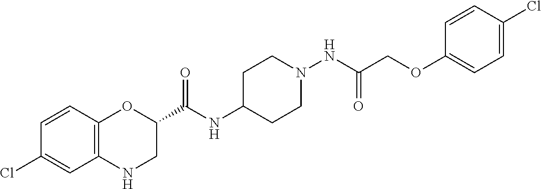 [SMILES: O=C(COc1ccc(Cl)cc1)NN1CCC(NC(=O)[C@@H]2CNc3cc(Cl)ccc3O2)CC1] |
| XX-73 | 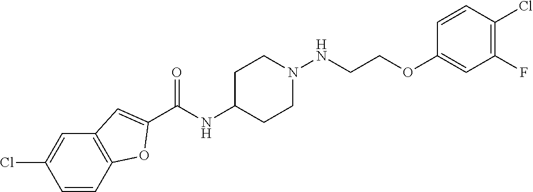 [SMILES: O=C(NC1CCN(NCCOc2ccc(Cl)c(F)c2)CC1)c1cc2cc(Cl)ccc2o1] |
| XX-74 | 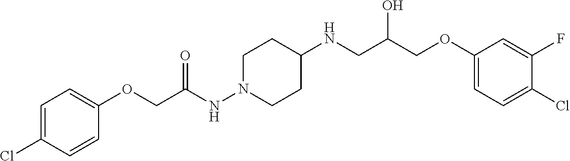 [SMILES: O=C(COc1ccc(Cl)cc1)NN1CCC(NCC(O)COc2ccc(Cl)c(F)c2)CC1] |

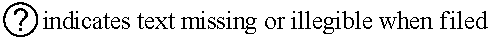

### Compositions and Formulations

Compositions of any of the compounds detailed herein are embraced by this disclosure. Thus, the present disclosure includes agricultural compositions comprising a compound as detailed herein or a agriculturally acceptable salt thereof and a agriculturally acceptable carrier or excipient. In one aspect, the agriculturally acceptable salt is an acid addition salt, such as a salt formed with an inorganic or organic acid. Agricultural compositions may take a form suitable for applying to a plant, such as a for suitable for spraying, chemigation (applying the composition through an irrigation system), granular application, or applying to fertilizer.

Agricultural compositions disclosed herein may comprise excipents or adjuvants, such as solvents, anti-caking agents, stabilizers, defoamers, slip agents, humectants, dispersants, wetting agents, thickening agents, emulsifiers, and preservatives. The agricultural composition may be a concentrated formulation or a ready-to-use formulation.

Pharmaceutical compositions of any of the compounds detailed herein are embraced by this disclosure. Thus, the present disclosure includes pharmaceutical compositions comprising a compound as detailed herein or a salt thereof and a pharmaceutically acceptable carrier or excipient. In one aspect, the pharmaceutically acceptable salt is an acid addition salt, such as a salt formed with an inorganic or organic acid. Pharmaceutical compositions may take a form suitable for oral, buccal, parenteral, nasal, topical or rectal administration or a form suitable for administration by inhalation.

A compound as detailed herein may in one aspect be in a purified form and compositions comprising a compound in purified forms are detailed herein. Compositions comprising a compound as detailed herein or a salt thereof are provided, such as compositions of substantially pure compounds. In some embodiments, a composition containing a compound as detailed herein or a salt thereof is in substantially pure form.

In one variation, the compounds herein are synthetic compounds prepared for administration to an individual. In another variation, compositions are provided containing a compound in substantially pure form. In another variation, the present disclosure embraces pharmaceutical compositions comprising a compound detailed herein and a pharmaceutically acceptable carrier. In another variation, methods of administering a compound are provided. The purified forms, pharmaceutical compositions and methods of administering the compounds are suitable for any compound or form thereof detailed herein.

A compound detailed herein or salt thereof may be formulated for any available delivery route, including an oral, mucosal (e.g., nasal, sublingual, vaginal, buccal or rectal), parenteral (e.g., intramuscular, subcutaneous or intravenous), topical or transdermal delivery form. A compound or salt thereof may be formulated with suitable carriers to provide delivery forms that include, but are not limited to, tablets, caplets, capsules (such as hard gelatin capsules or soft elastic gelatin capsules), cachets, troches, lozenges, gums, dispersions, suppositories, ointments, cataplasms (poultices), pastes, powders, dressings, creams, solutions, patches, aerosols (e.g., nasal spray or inhalers), gels, suspensions (e.g., aqueous or non-aqueous liquid suspensions, oil-in-water emulsions or water-in-oil liquid emulsions), solutions and elixirs.

One or several compounds described herein or a salt thereof can be used in the preparation of a formulation, such as a pharmaceutical formulation, by combining the compound or compounds, or a salt thereof, as an active ingredient with a pharmaceutically acceptable carrier, such as those mentioned above. Depending on the therapeutic form of the system (e.g., transdermal patch vs. oral tablet), the carrier may be in various forms. In addition, pharmaceutical formulations may contain preservatives, solubilizers, stabilizers, re-wetting agents, emulgators, sweeteners, dyes, adjusters, and salts for the adjustment of osmotic pressure, buffers, coating agents or antioxidants. Formulations comprising the compound may also contain other substances which have valuable therapeutic properties. Pharmaceutical formulations may be prepared by known pharmaceutical methods. Suitable formulations can be found, e.g., in Remington's Pharmaceutical Sciences, Mack Publishing Company, Philadelphia, Pa., 20^{th} ed. (2000), which is incorporated herein by reference.

Compounds as described herein may be administered to individuals in a form of generally accepted oral compositions, such as tablets, coated tablets, and gel capsules in a hard or in soft shell, emulsions or suspensions. Examples of carriers, which may be used for the preparation of such compositions, are lactose, corn starch or its derivatives, talc, stearate or its salts, etc. Acceptable carriers for gel capsules with soft shell are, for instance, plant oils, wax, fats, semisolid and liquid poly-ols, and so on. In addition, pharmaceutical formulations may contain preservatives, solubilizers, stabilizers, re-wetting agents, emulgators, sweeteners, dyes, adjusters, and salts for the adjustment of osmotic pressure, buffers, coating agents or antioxidants.

Any of the compounds described herein can be formulated in a tablet in any dosage form described, for example, a compound as described herein or a salt thereof can be formulated as a 10 mg tablet.

Compositions comprising a compound provided herein are also described. In one variation, the composition comprises a compound or salt thereof and a pharmaceutically acceptable carrier or excipient. In another variation, a composition of substantially pure compound is provided. In some embodiments, the composition is for use as a human or veterinary medicament. In some embodiments, the composition is for use in a method described herein. In some embodiments, the composition is for use in the treatment of a disease or disorder described herein.

### Methods of Use and Uses

Compounds and compositions detailed herein, such as a pharmaceutical composition containing a compound of any formula provided herein or a salt thereof and a pharmaceutically acceptable carrier or excipient, may be used in methods of administration and treatment as provided herein. The compounds and compositions may also be used in in vitro methods, such as in vitro methods of administering a compound or composition to cells for screening purposes and/or for conducting quality control assays.

Provided herein is a method of treating a disease or disorder in an individual in need thereof comprising administering a compound describes herein or any embodiment, variation, or aspect thereof, or a pharmaceutically acceptable salt thereof. In some embodiments, the compound, pharmaceutically acceptable salt thereof, or composition is administered to the individual according to a dosage and/or method of administration described herein.

The compounds or salts thereof described herein and compositions described herein are believed to be effective for treating a variety of diseases and disorders. In some embodiments, a compound or salt thereof described herein or a composition described herein may be used in a method of treating a disease or disorder mediated by an integrated stress response (ISR) pathway. In some embodiments, the disease or disorder is mediated by eukaryotic translation initiation factor 2α (eIF2α) or eukaryotic translation initiation factor 2B (eIF2B). In some embodiments, the disease or disorder is mediated by phosphorylation of eIF2α and/or the guanine nucleotide exchange factor (GEF) activity of eIF2B.

In some embodiments, a compound or salt thereof described herein or a composition described herein may be used in a method of treating a disease or disorder, wherein the disease or disorder is a neurodegenerative disease, an inflammatory disease, an autoimmune disease, a metabolic syndrome, a cancer, a vascular disease, a musculoskeletal disease (such as a myopathy), an ocular disease, or a genetic disorder.

In some embodiments, the disease or disorder is a neurodegenerative disease. In some embodiments, the neurodegenerative disease is vanishing white matter disease, childhood ataxia with CNS hypomyelination, intellectual disability syndrome, Alzheimer's disease, prion disease, Creutzfeldt-Jakob disease, Parkinson's disease, amyotrophic lateral sclerosis (ALS) disease, Pelizaeus-Merzbacher disease, a cognitive impairment, a traumatic brain injury, a postoperative cognitive dysfunction (PCD), a neuro-otological syndrome, hearing loss, Huntington's disease, stroke, chronic traumatic encephalopathy, spinal cord injury, dementia, frontotemporal dementia (FTD), depression, or a social behavior impairment. In some embodiments, the cognitive impairment is triggered by ageing, radiation, sepsis, seizure, heart attack, heart surgery, liver failure, hepatic encephalopathy, anesthesia, brain injury, brain surgery, ischemia, chemotherapy, cancer treatment, critical illness, concussion, fibromyalgia, or depression. In some embodiments, the neurodegenerative disease is Alzheimer's disease. In some embodiments, the neurodegenerative disease is ageing-related cognitive impairment. In some embodiments, the neurodegenerative disease is a traumatic brain injury.

In some embodiments, a compound or salt thereof described herein or a composition described herein may be used in a method of treating Alzheimer's disease. In some embodiments, neurodegeneration, cognitive impairment, and/or amyloidogenesis is decreased.

In some embodiments, the disease or disorder is an inflammatory disease. In some embodiments, the inflammatory disease is arthritis, psoriatic arthritis, psoriasis, juvenile idiopathic arthritis, asthma, allergic asthma, bronchial asthma, tuberculosis, chronic airway disorder, cystic fibrosis, glomerulonephritis, membranous nephropathy, sarcoidosis, vasculitis, ichthyosis, transplant rejection, interstitial cystitis, atopic dermatitis, or inflammatory bowel disease. In some embodiments, the inflammatory bowel disease is Crohn' disease, ulcerative colitis, or celiac disease.

In some embodiments, the disease or disorder is an autoimmune disease. In some embodiments, the autoimmune disease is systemic lupus erythematosus, type 1 diabetes, multiple sclerosis, or rheumatoid arthritis.

In some embodiments, the disease or disorder is a metabolic syndrome. In some embodiments, the metabolic syndrome is acute pancreatitis, chronic pancreatitis, alcoholic liver steatosis, obesity, glucose intolerance, insulin resistance, hyperglycemia, fatty liver, dyslipidemia, hyperlipidemia, hyperhomocysteinemia, or type 2 diabetes. In some embodiments, the metabolic syndrome is alcoholic liver steatosis, obesity, glucose intolerance, insulin resistance, hyperglycemia, fatty liver, dyslipidemia, hyperlipidemia, hyperhomocysteinemia, or type 2 diabetes.

In some embodiments, the disease or disorder is a cancer. In some embodiments, the cancer is pancreatic cancer, breast cancer, kidney cancer, bladder cancer, prostate cancer, testicular cancer, urothelial cancer, endometrial cancer, ovarian cancer, cervical cancer, renal cancer, esophageal cancer, gastrointestinal stromal tumor (GIST), multiple myeloma, cancer of secretory cells, thyroid cancer, gastrointestinal carcinoma, chronic myeloid leukemia, hepatocellular carcinoma, colon cancer, melanoma, malignant glioma, glioblastoma, glioblastoma multiforme, astrocytoma, dysplastic gangliocytoma of the cerebellum, Ewing's sarcoma, rhabdomyosarcoma, ependymoma, medulloblastoma, ductal adenocarcinoma, adenosquamous carcinoma, nephroblastoma, acinar cell carcinoma, neuroblastoma, or lung cancer. In some embodiments, the cancer of secretory cells is non-Hodgkin's lymphoma, Burkitt's lymphoma, chronic lymphocytic leukemia, monoclonal gammopathy of undetermined significance (MGUS), plasmacytoma, lymphoplasmacytic lymphoma or acute lymphoblastic leukemia.

In some embodiments, the disease or disorder is a musculoskeletal disease (such as a myopathy). In some embodiments, the musculoskeletal disease is a myopathy, a muscular dystrophy, a muscular atrophy, a muscular wasting, or sarcopenia. In some embodiments, the muscular dystrophy is Duchenne muscular dystrophy (DMD), Becker's disease, myotonic dystrophy, X-linked dilated cardiomyopathy, spinal muscular atrophy (SMA), or metaphyseal chondrodysplasia, Schmid type (MCDS). In some embodiments, the myopathy is a skeletal muscle atrophy. In some embodiments, the musculoskeletal disease (such as the skeletal muscle atrophy) is triggered by ageing, chronic diseases, stroke, malnutrition, bedrest, orthopedic injury, bone fracture, cachexia, starvation, heart failure, obstructive lung disease, renal failure, Acquired Immunodeficiency Syndrome (AIDS), sepsis, an immune disorder, a cancer, ALS, a burn injury, denervation, diabetes, muscle disuse, limb immobilization, mechanical unload, myositis, or a dystrophy.

In some embodiments, the disease or disorder is a genetic disorder, such as Down syndrome or MEHMO syndrome (Mental retardation, Epileptic seizures, Hypogenitalism, Microcephaly, and Obesity).

In some embodiments, a compound or salt thereof described herein or a composition described herein may be used in a method of treating musculoskeletal disease. In some embodiments, skeletal muscle mass, quality and/or strength are increased. In some embodiments, synthesis of muscle proteins is increased. In some embodiments, skeletal muscle fiber atrophy is inhibited.

In some embodiments, the disease or disorder is a vascular disease. In some embodiments, the vascular disease is atherosclerosis, abdominal aortic aneurism, carotid artery disease, deep vein thrombosis, Buerger's disease, chronic venous hypertension, vascular calcification, telangiectasia or lymphoedema.

In some embodiments, the disease or disorder is an ocular disease. In some embodiments, the ocular disease is glaucoma, age-related macular degeneration, inflammatory retinal disease, retinal vascular disease, diabetic retinopathy, uveitis, rosacea, Sjogren's syndrome, or neovascularization in proliferative retinopathy.

In some embodiments, provided herein is a method of modulating an ISR pathway. The compounds or salts thereof described herein and compositions described herein are believed to be effective for modulating an ISR pathway. In some embodiments, the method of modulating an ISR pathway comprises modulating the ISR pathway in a cell by administering or delivering to the cell a compound described herein, or a pharmaceutically acceptable salt thereof, or a pharmaceutical composition described herein. In some embodiments, the method of modulating an ISR pathway comprises modulating the ISR pathway in an individual by administering to the individual a compound described herein, or a pharmaceutically acceptable salt thereof, or a pharmaceutical composition described herein. modulating of the ISR pathway can be determined by methods known in the art, such as western blot, immunohistochemistry, or reporter cell line assays.

In some embodiments, the modulation of the ISR pathway comprises binding eIF2B. In some embodiments, the modulation of the ISR pathway comprises increasing protein translation, increasing guanine nucleotide exchange factor (GEF) activity of eIF2B, delaying or preventing apoptosis in a cell, and/or modulating translation of one or more mRNAs comprising a 5′ untranslated region (5′UTR) comprising at least one upstream open reading frame (uORF).

In some embodiments, provided herein are methods of increasing protein production using a compound or salt described herein. The protein production is increased relative to the same condition without the compound or salt. Protein production can be increased either in vivo or in vitro. For example, protein production can be increased in vivo by administering the compound or salt to an individual. In some embodiments, protein production is increased in vitro using the compound or salt with a cell-free protein synthesis system (CFPS) or a cell-based protein expression system. The protein produced can be a heterologous protein (e.g., a recombinant protein) or a native protein. Heterologous protein production can be achieved using a recombinant nucleic acid encoding the protein. In some embodiments, the protein produced is an antibody or a fragment thereof. Other exemplary proteins can include, but are not limited to, enzymes, allergenic peptides or proteins (for example, for use as a vaccine), recombinant protein, cytokines, peptides, hormones, erythropoietin (EPO), interferons, granulocyte-colony stimulating factor (G-CSF), anticoagulants, and clotting factors. The increase in protein production can be determined by methods known in the art, such as western blot or immunohistochemistry.

Cell-free protein synthesis (CFPS) systems are generally known, and include cellular machinery for protein expression in an in vitro environment. In some embodiments, the CFPS system includes a cellular extract (such as a eukaryotic cellular extract), which includes protein expression machinery. In some embodiment, the cellular machinery in the CFPS system comprises eukaryotic cellular machinery, such as eukaryotic initiation factor 2 (eIF2) and/or eukaryotic initiation factor 2B (eIF2B), or one or more subunits thereof.

In some embodiments, there is a cell-free protein synthesis (CFPS) system comprising eukaryotic initiation factor 2 (eIF2) and a nucleic acid encoding a protein with a compound or salt as described herein. In some embodiments, the protein is an antibody or a fragment thereof. Other exemplary proteins can include, but are not limited to, enzymes, allergenic peptides or proteins (for example, for use as a vaccine), recombinant protein, cytokines, peptides, hormones, erythropoietin (EPO), interferons, granulocyte-colony stimulating factor (G-CSF), anticoagulants, and clotting factors. In some embodiments, the CFPS system comprises a cell extract comprising the eIF2. In some embodiments, the CFPS system further comprises eIF2B.

In some embodiments, there is a method of producing a protein, comprising contacting a cell-free protein synthesis (CFPS) system comprising eukaryotic initiation factor 2 (eIF2) and a nucleic acid encoding a protein with a compound or salt thereof as described herein. In some embodiments, the protein is an antibody or a fragment thereof. Other exemplary proteins can include, but are not limited to, enzymes, allergenic peptides or proteins (for example, for use as a vaccine), recombinant protein, cytokines, peptides, hormones, erythropoietin (EPO), interferons, granulocyte-colony stimulating factor (G-CSF), anticoagulants, and clotting factors. In some embodiments, the CFPS system comprises a cell extract comprising the eIF2. In some embodiments, the CFPS system further comprises eIF2B. In some embodiments, the method comprises purifying the protein.

In some embodiments, there is a method of producing a protein, comprising contacting a eukaryotic cell comprising a nucleic acid encoding the protein with a compound or salt as described herein. In some embodiments, the method comprises culturing the cell in an in vitro culture medium comprising the compound or salt. In some embodiments, the nucleic acid encoding the protein is a recombinant nucleic acid. In some embodiments, the eukaryotic cell is a human embryonic kidney (HEK) cell or a Chinese hamster ovary (CHO) cell. In other embodiments, the eukaryotic cell is a yeast cell (such as Saccharomyces cerevisiae or Pichia pastoris), a wheat germ cell, an insect cell, a rabbit reticulocyte, a cervical cancer cell (such as a HeLa cell), a baby hamster kidney cell (such as BHK21 cells), a murine myeloma cell (such as NSO or Sp2/0 cells), an HT-1080 cell, a PER.C6 cell, a plant cell, a hybridoma cell, or a human blood derived leukocyte. In some embodiments, the protein is an antibody or a fragment thereof. Other exemplary proteins can include, but are not limited to, enzymes, allergenic peptides or proteins (for example, for use as a vaccine), recombinant protein, cytokines, peptides, hormones, erythropoietin (EPO), interferons, granulocyte-colony stimulating factor (G-CSF), anticoagulants, and clotting factors. In some embodiments, the method comprises purifying the protein.

In some embodiments, there is a method of culturing a eukaryotic cell comprising a nucleic acid encoding a protein, comprising contacting the eukaryotic cell with an in vitro culture medium comprising a compound or salt as described herein. In some embodiments, the nucleic acid encoding the protein is a recombinant nucleic acid. In some embodiments, the eukaryotic cell is a human embryonic kidney (HEK) cell or a Chinese hamster ovary (CHO) cell. In other embodiments, the eukaryotic cell is a yeast cell (such as Saccharomyces cerevisiae or Pichia pastoris), a wheat germ cell, an insect cell, a rabbit reticulocyte, a cervical cancer cell (such as a HeLa cell), a baby hamster kidney cell (such as BHK21 cells), a murine myeloma cell (such as NSO or Sp2/0 cells), an HT-1080 cell, a PER.C6 cell, a plant cell, a hybridoma cell, or a human blood derived leukocyte. In some embodiments, the protein is an antibody or a fragment thereof. Other exemplary proteins can include, but are not limited to, enzymes, allergenic peptides or proteins (for example, for use as a vaccine), recombinant protein, cytokines, peptides, hormones, erythropoietin (EPO), interferons, granulocyte-colony stimulating factor (G-CSF), anticoagulants, and clotting factors. In some embodiments, the method comprises purifying the protein.

In some embodiments, there is an in vitro cell culture medium, comprising the compound or salt described herein, and nutrients for cellular growth. In some embodiments, the culture medium comprises a eukaryotic cell comprising a nucleic acid encoding a protein. In some embodiments, the culture medium further comprises a compound for inducing protein expression. In some embodiments, the nucleic acid encoding the protein is a recombinant nucleic acid. In some embodiments, the protein is an antibody or a fragment thereof. Other exemplary proteins can include, but are not limited to, enzymes, allergenic peptides or proteins (for example, for use as a vaccine), recombinant protein, cytokines, peptides, hormones, erythropoietin (EPO), interferons, granulocyte-colony stimulating factor (G-CSF), anticoagulants, and clotting factors. In some embodiments, the eukaryotic cell is a human embryonic kidney (HEK) cell or a Chinese hamster ovary (CHO) cell. In other embodiments, the eukaryotic cell is a yeast cell (such as Saccharomyces cerevisiae or Pichia pastoris), a wheat germ cell, an insect cell, a rabbit reticulocyte, a cervical cancer cell (such as a HeLa cell), a baby hamster kidney cell (such as BHK21 cells), a murine myeloma cell (such as NSO or Sp2/0 cells), an HT-1080 cell, a PER.C6 cell, a plant cell, a hybridoma cell, or a human blood derived leukocyte.

In some embodiments, provided herein is a method of increasing protein translation in a cell or cell free expression system. In some embodiments, the cell was stressed prior to administration of the compound, salt thereof, or composition. In some embodiments, protein translation is increased by at least about 10%, 20%, 30%, 40%, 50%, 60%, 70%, 80%, 90%, 95%, 98%, 100%, 125%, 150%, 175%, 200%, 250%, or 300% or more. In some embodiments, protein translation is increased by about 10% to about 300% (such as about 10% to about 20%, about 20% to about 30%, about 30% to about 40%, about 40% to about 50%, about 50% to about 60%, about 60% to about 70%, about 70% to about 80%, about 80% to about 90%, about 90% to about 100%, about 100% to about 125%, about 125% to about 150%, about 150% to about 175%, about 175% to about 200%, about 200% to about 250%, or about 250% to about 300%) In some embodiments, protein translation is increased as compared to prior to the administration of the compounds, salt thereof, or composition. In some embodiments, protein translation is increased as compared to an unstressed cell, a basal condition where cells are not subjected to a specific stress that activates the ISR. In some embodiments, protein translation is increased as compared to a stressed cell where ISR is active.

The compounds described herein increase protein synthesis in a cell without full inhibition of ATF4 translation, under ISR-stressed or non-ISR stressed conditions. Despite ATF4 participation in various pathologies, the ATF4 protein is an important factor for restoring cellular homeostasis in stressed cells, for example during oxidative stress response, cholesterol metabolism, protein folding amino acid synthesis, and autophagy. Thus, for certain treatments, it may be preferable to limit or avoid ATF4 inhibition. In some embodiments, the compound is used to increase protein synthesis by about 10% or more, about 20% or more, about 30% or more, about 40% or more, about 50% or more, about 60% or more, about 70% or more, about 80% or more, about 90% or more, about 100% or more, about 125% or more, about 150% or more, about 175% or more, about 200% or more, about 250% or more, or about 300% or more wherein ATF4 protein expression is not substantially inhibited or is inhibited by about 75% or less, about 50% or less, about 40% or less, about 30% or less, about 20% or less, about 10% or less, or about 5% or less. In some embodiments the compound is used to increase protein synthesis by about 10% to about 1000% (such as about 10% to about 20%, about 20% to about 30%, about 30% to about 40%, about 40% to about 50%, about 50% to about 60%, about 60% to about 70%, about 70% to about 80%, about 80% to about 90%, about 90% to about 100%, about 100% to about 125%, about 125% to about 150%, about 150% to about 175%, about 175% to about 200%, about 200% to about 250%, about 250% to about 300%, about 300% to about 350%, about 350% to about 400%, about 400% to about 450%, about 450% to about 500%, about 500% to about 600%, about 600% to about 700%, about 700% to about 800%, about 800% to about 900%, or about 900% to about 1000%), wherein ATF4 protein expression is not substantially inhibited or is inhibited by about 75% or less (such as about 50% or less, about 40% or less, about 30% or less, about 20% or less, about 10% or less, or about 5% or less).

In some embodiments, provided herein is a method of increasing protein translation in a cell. In some embodiments, the cell was stressed prior to administration of the compound, salt thereof, or composition. In some embodiments, protein translation is increased by at least about 10%, 20%, 30%, 40%, 50%, 60%, 70%, 80%, 90%, 95%, 98%, 100%, 125%, 150%, 175%, 200%, 250%, or 300% or more. In some embodiments, protein translation is increased as compared to prior to the administration of the compounds, salt thereof, or composition. In some embodiments, protein translation is increased as compared to an unstressed cell, a basal condition where cells are not subjected to a specific stress that activates the ISR. In some embodiments, protein translation is increased as compared to a stressed cell where ISR is active.

In some embodiments, provided herein is a method of increasing guanine nucleotide exchange factor (GEF) activity of eIF2B in cells. In some embodiments, provided herein is a method of delaying or preventing apoptosis in a cell. In some embodiments, provided herein is a method of inhibiting translation of one or more mRNAs comprising a 5′ untranslated region (5′UTR) that contains at least one upstream open reading frame (uORF), encoding proteins with translational preferences, including but not limited to ATF4, ATF2, ATF5, ATF3, FGF-21, CHOP, GADD34, BACE-1, C/EBPα, or MAP1LC3B. In some embodiments, the mRNA encodes ATF4, ATF3, FGF-21, BACE-1, GADD34, or CHOP. In some embodiments, the mRNA encodes ATF4, ATF2, ATF5, CHOP, GADD34, BACE-1, C/EBPα, or MAP1LC3B. In some embodiments, the mRNA encodes ATF4, BACE-1, GADD34, or CHOP. In some embodiments, the mRNA encodes ATF4.

In some embodiments, expression of ATF4, BACE-1, GADD34 or CHOP is inhibited. In some embodiments, expression of ATF4 is inhibited. In some embodiments, expression of A is inhibited. ATF4 increases expression of, among others, GADD45A, CDKN1A, and EIF4EBP1, which encode DDIT-1, p21, and 4E-BP1, respectively. These proteins induce musculoskeletal disease (such as skeletal muscle atrophy), and can be modulated by inhibiting expression of ATF4. Accordingly, in some embodiments, expression of one or more of CDKN1A, GADD45A, or EIF4EBP1 is inhibited.

In some embodiments, the compound, salt thereof, or composition inhibits translation of one or more mRNAs comprising a 5′ untranslated region (5′UTR) comprising at least one upstream open reading frame (uORF) with an IC₅₀ of less than about 100 μM, such as less than about 75 μM, about 50 μM, about 25 μM, about 20 μM, about 10 μM, about 5 μM, about 1 μM, about 750 nM, 600 nM, 500 nM, 300 nM, 200 nM, 100 nM, 80 nM, 60 nM, 40 nM, 25 nM, or less. In some embodiments, the compound, salt thereof, or composition inhibits translation of one or more mRNAs comprising a 5′ untranslated region (5′UTR) comprising at least one upstream open reading frame (uORF) with an IC₅₀ between about 1 nM and 100 μM, such as between about 10 nM and 600 nM, 15 nM and 200 nM, or 20 nM and 180 nM.

In some embodiments, the compound, salt thereof, or composition inhibits expression of ATF4 with an IC₅₀ of less than about 100 μM, such as less than about 75 μM, about 50 μM, about 25 μM, about 20 μM, about 10 μM, about 5 μM, about 1 μM, about 750 nM, 600 nM, 500 nM, 300 nM, 200 nM, 100 nM, 80 nM, 60 nM, 40 nM, 25 nM, or less. In some embodiments, the compound, salt thereof, or composition inhibits expression of ATF4 with an IC₅₀ between about 1 nM and 100 μM, such as between about 2 nM and 800 nM, 10 nM and 600 nM, 15 nM and 200 nM, or 20 nM and 180 nM.

In some aspects, the half maximal inhibitory concentration (IC₅₀) is a measure of the effectiveness of a substance in inhibiting a specific biological or biochemical function. In some aspects, the IC₅₀ is a quantitative measure that indicates how much of an inhibitor is needed to inhibit a given biological process or component of a process such as an enzyme, cell, cell receptor or microorganism by half. Methods of determining IC₅₀ in vitro and in vivo are known in the art.

In some embodiments, the individual is a mammal. In some embodiments, the individual is a primate, bovine, ovine, porcine, equine, canine, feline, rabbit, or rodent. In some embodiments, the individual is a human. In some embodiments, the individual has any of the diseases or disorders disclosed herein. In some embodiments, the individual is a risk for developing any of the diseases or disorders disclosed herein.

In some embodiments, the individual is human. In some embodiments, the human is at least about or is about any of 21, 25, 30, 35, 40, 45, 50, 55, 60, 65, 70, 75, 80, or 85 years old. In some embodiments, the human is a child. In some embodiments, the human is less than about or about any of 21, 18, 15, 12, 10, 8, 6, 5, 4, 3, 2, or 1 years old.

Also provided herein are uses of a compound described herein or a pharmaceutically acceptable salt thereof, or a pharmaceutical composition described herein, in the manufacture of a medicament. In some embodiments, the manufacture of a medicament is for the treatment of a disorder or disease described herein. In some embodiments, the manufacture of a medicament is for the prevention and/or treatment of a disorder or disease mediated by an ISR pathway. In some embodiments, the manufacture of a medicament is for the prevention and/or treatment of a disorder or disease mediated by eIF2α or eIF2B. In some embodiments, the manufacture of a medicament is for the prevention and/or treatment of a disorder or disease mediated by phosphorylation of eIF2α and/or the GEF activity of eIF2B.

In some embodiments, there is a method for enhancing protein synthesis in a living organism, comprising administering to the living organism an effective amount of a compound or salt thereof as provided herein. In some embodiments, the living organism is selected from the group consisting of a cell suspension, a hairy root culture, moss protonema, an aquatic plant (including but not limited to duckweed and microalgae), and a terrestrial plant. In some embodiments, the living organism is a terrestrial plant. In some embodiments, the terrestrial plant is selected from soybean, sunflower, grain legume, rice, wheat germ, maize, tobacco, a cereal, and a lupin crop. In some embodiments, the terrestrial plant is tobacco.

In some embodiments, provided is a method for producing a protein in a living organism, comprising contacting the living organism with a compound described herein or a salt thereof (such as an agriculturally acceptable salt thereof), and wherein the protein is selected from the group consisting of a biopolymer, an industrial protein, an industrial enzyme, and a therapeutic protein. In some embodiments, the living organism is selected from the group consisting of a cell suspension, a hairy root culture, moss protonema, an aquatic plant (including but not limited to duckweed and microalgae), and a terrestrial plant. In some embodiments, the living organism is a terrestrial plant. In some embodiments, the terrestrial plant is tobacco. In some embodiments, the protein is an industrial protein selected from the group consisting of a hydrolase, a glycosidase (such as a cellulase, and α-amylase, a β-glucuronidase, and the likes), a protease (such as trypsin), and the likes. In some embodiments, the protein is a therapeutic protein selected from the group consisting of an antibody, a vaccine, a human growth-factor, a cytokine, and the likes.

In some embodiments, there is a method for accelerating growth of a plant, comprising administering to the plant an effective amount of a compound or salt thereof as provided herein. In some embodiments, the plant is an aquatic plant. In some embodiments, the plant is a terrestrial plant. In some embodiments, the terrestrial plant is selected from soybean, sunflower, grain legume, rice, wheat germ, maize, tobacco, a cereal, and a lupin crop. In some embodiments, the terrestrial plant is tobacco.

In some embodiments, there is a method for improving protein yield or quality in a plant, comprising administering to the plant an effective amount of a compound or salt thereof as provided herein. In some embodiments, the plant is an aquatic plant. In some embodiments, the plant is a terrestrial plant. In some embodiments, the terrestrial plant is selected from soybean, sunflower, grain legume, rice, wheat germ, maize, tobacco, a cereal, and a lupin crop. In some embodiments, the terrestrial plant is tobacco.

### Combinations

In certain aspects, a compound described herein is administered to an individual for treatment of a disease in combination with one or more additional pharmaceutical agents that can treat the disease. For example, in some embodiments, an effective amount of the compound is administered to an individual for the treatment of cancer in combination with one or more additional anticancer agents.

In some embodiments, activity of the additional pharmaceutical agent (such as additional anticancer agent) is inhibited by an activated ISR pathway. An ISR inhibitor, such as one of the compounds described herein, can inhibit the ISR pathway to enhance functionality of the additional pharmaceutical agent. By way of example, certain BRAF inhibitors (e.g., vemurafenib or dabrafenib) activate the ISR pathway in BRAF-mutated melanoma cells (e.g., BRAF with a V600F mutation) through the expression of ATF4. In some embodiments, there is a method of treating cancer comprising administering to an individual with cancer an effective amount of a compound described herein in combination with an effective amount of a BRAF inhibitor. In some embodiments, there is a method of treating a BRAF-mutated melanoma comprising administering to an individual with a BRAF-mutated melanoma an effective amount of a compound described herein in combination with an effective amount of a BRAF inhibitor. In some embodiments, there is a method of treating a BRAF-mutated melanoma comprising administering to an individual with a BRAF-mutated melanoma an effective amount of a compound described herein in combination with an effective amount of vemurafenib or dabrafenib.

As another example, certain anticancer agents (such as ubiquitin-proteasome pathway inhibitors (such as bortezomib), Cox-2 inhibitors (e.g., celecoxib), platinum-based antineoplastic drugs (e.g., cisplatin), anthracyclines (e.g. doxorubicin), or topoisomerase inhibitors (e.g., etoposide)) are used to treat cancer, but may have limited functionality against solid tumors. Resistance in certain solid tumors (e.g., breast cancers) has been associated with ATF4 stabilization and induction of autophagy. In some embodiments, an effective amount of an ISR inhibitor compound as described herein is administered to an individual with cancer to increase sensitivity to one or more anticancer agents.

In some embodiments, there is a method of treating a refractory cancer (such as a solid tumor) in an individual, comprising administering to the individual an effective amount of a compound described herein in combination with an effective amount of an anticancer agent. In some embodiments, there is a method of treating a refractory cancer (such as a solid tumor) in an individual, comprising administering to the individual an effective amount of a compound described herein in combination with an effective amount of an ubiquitin-proteasome pathway inhibitor (e.g., bortezomib), a Cox-2 inhibitor (e.g., celecoxib), a platinum-based antineoplastic drug (e.g., cisplatin), an anthracycline (e.g. doxorubicin), or a topoisomerase inhibitor (e.g., etoposide). In some embodiments, the refractory cancer is breast cancer. In some embodiments, the refractory cancer is melanoma.

In some embodiments, a compound described herein is used to treat cancer in combination with one or more anti-cancer agents, such as an anti-neoplastic agent, an immune checkpoint inhibitor, or any other suitable anti-cancer agent. Exemplary immune checkpoint inhibitors include anti-PD-1, anti-PD-L¹, anti GITR, anti-OX-40, anti-LAG3, anti-TIM-3, anti-41BB, anti-CTLA-4 antibodies. Exemplary anti-neoplastic agents can include, for example, anti-microtubule agents, platinum coordination complexes, alkylating agents, topoisomerase II inhibitors, topoisomerase I inhibitors, antimetabolites, antibiotic agents, hormones and hormonal analogs, signal transduction pathway inhibitors, non-receptor tyrosine kinase angiogenesis inhibitors, proteasome inhibitors, and inhibitors of cancer metabolism. Other anti-cancer agents can include one or more of an immuno-stimulant, an antibody or fragment thereof (e.g., an anti-CD20, anti-HER2, anti-CD52, or anti-VEGF antibody or fragment thereof), or an immunotoxin (e.g., an anti-CD33 antibody or fragment thereof, an anti-CD22 antibody or fragment thereof, a calicheamicin conjugate, or a pseudomonas exotoxin conjugate).

ATF4-mediated expression of CHOP has also been shown to regulate the function and accumulation of myeloid-derived suppressor cells (MDSCs) in tumors. MDSCs in tumors reduce the ability to prime T cell function and reduce antitumoral or anticancer responses. Certain immunotherapeutic agents (such as anti-PD-1, anti PD-L, anti-GITR, anti-OX-40, anti-LAG3, anti-TIM-3, anti-41BB, or anti-CTLA-4 antibodies) have been used to boost the immune response against cancer. ATF4-mediated expression of AXL has been associated with poor response to anti-PD1 therapy in melanoma. In some embodiments, an effective amount of an ISR inhibitor compound as described herein is administered to an individual with cancer to increase sensitivity to one or more immunotherapeutic agents. In some embodiments, there is a method of treating a refractory cancer (such as a melanoma) in an individual, comprising administering to the individual an effective amount of a compound described herein in combination with an effective amount of an immunotherapeutic agent (e.g. anti-PD-1, anti PD-L¹, anti-GITR, anti-OX-40, anti-LAG3, anti-TIM-3, anti-41BB, or anti-CTLA-4 antibodies). In some embodiments, the refractory cancer is melanoma.

### Dosing and Method of Administration

The dose of a compound administered to an individual (such as a human) may vary with the particular compound or salt thereof, the method of administration, and the particular disease, such as type and stage of cancer, being treated. In some embodiments, the amount of the compound or salt thereof is a therapeutically effective amount.

The effective amount of the compound may in one aspect be a dose of between about 0.01 and about 100 mg/kg. Effective amounts or doses of the compounds of the present disclosure may be ascertained by routine methods, such as modeling, dose escalation, or clinical trials, taking into account routine factors, e.g., the mode or route of administration or drug delivery, the pharmacokinetics of the agent, the severity and course of the disease to be treated, the subject's health status, condition, and weight. An exemplary dose is in the range of about from about 0.7 mg to 7 g daily, or about 7 mg to 350 mg daily, or about 350 mg to 1.75 g daily, or about 1.75 to 7 g daily.

Any of the methods provided herein may in one aspect comprise administering to an individual a pharmaceutical composition that contains an effective amount of a compound provided herein or a salt thereof and a pharmaceutically acceptable excipient.

A compound or composition provided herein may be administered to an individual in accordance with an effective dosing regimen for a desired period of time or duration, such as at least about one month, at least about 2 months, at least about 3 months, at least about 6 months, or at least about 12 months or longer, which in some variations may be for the duration of the individual's life. In one variation, the compound is administered on a daily or intermittent schedule. The compound can be administered to an individual continuously (for example, at least once daily) over a period of time. The dosing frequency can also be less than once daily, e.g., about a once weekly dosing. The dosing frequency can be more than once daily, e.g., twice or three times daily. The dosing frequency can also be intermittent, including a ‘drug holiday’ (e.g., once daily dosing for 7 days followed by no doses for 7 days, repeated for any 14 day time period, such as about 2 months, about 4 months, about 6 months or more). Any of the dosing frequencies can employ any of the compounds described herein together with any of the dosages described herein.

### Articles of Manufacture and Kits

The present disclosure further provides articles of manufacture comprising a compound described herein or a salt thereof, a composition described herein, or one or more unit dosages described herein in suitable packaging. In certain embodiments, the article of manufacture is for use in any of the methods described herein. Suitable packaging is known in the art and includes, for example, vials, vessels, ampules, bottles, jars, flexible packaging and the like. An article of manufacture may further be sterilized and/or sealed.

The present disclosure further provides kits for carrying out the methods of the present disclosure, which comprises one or more compounds described herein or a composition comprising a compound described herein. The kits may employ any of the compounds disclosed herein. In one variation, the kit employs a compound described herein or a salt thereof. The kits may be used for any one or more of the uses described herein, and, accordingly, may contain instructions for the treatment of any disease or described herein, for example for the treatment of cancer.

Kits generally comprise suitable packaging. The kits may comprise one or more containers comprising any compound described herein. Each component (if there is more than one component) can be packaged in separate containers or some components can be combined in one container where cross-reactivity and shelf life permit.

The kits may be in unit dosage forms, bulk packages (e.g., multi-dose packages) or subunit doses. For example, kits may be provided that contain sufficient dosages of a compound as disclosed herein and/or an additional pharmaceutically active compound useful for a disease detailed herein to provide effective treatment of an individual for an extended period, such as any of a week, 2 weeks, 3 weeks, 4 weeks, 6 weeks, 8 weeks, 3 months, 4 months, 5 months, 7 months, 8 months, 9 months, or more. Kits may also include multiple unit doses of the compounds and instructions for use and be packaged in quantities sufficient for storage and use in pharmacies (e.g., hospital pharmacies and compounding pharmacies).

The kits may optionally include a set of instructions, generally written instructions, although electronic storage media (e.g., magnetic diskette or optical disk) containing instructions are also acceptable, relating to the use of component(s) of the methods of the present disclosure. The instructions included with the kit generally include information as to the components and their administration to an individual.

### General Synthetic Methods

The compounds of the present disclosure may be prepared by a number of processes as generally described below and more specifically in the Examples hereinafter (such as the schemes provided in the Examples below). In the following process descriptions, the symbols when used in the formulae depicted are to be understood to represent those groups described above in relation to the formulae herein.

Where it is desired to obtain a particular enantiomer of a compound, this may be accomplished from a corresponding mixture of enantiomers using any suitable conventional procedure for separating or resolving enantiomers. Thus, for example, diastereomeric derivatives may be produced by reaction of a mixture of enantiomers, e.g., a racemate, and an appropriate chiral compound. The diastereomers may then be separated by any convenient means, for example by crystallization and the desired enantiomer recovered. In another resolution process, a racemate may be separated using chiral High-Performance Liquid Chromatography. Alternatively, if desired a particular enantiomer may be obtained by using an appropriate chiral intermediate in one of the processes described.

Chromatography, recrystallization and other conventional separation procedures may also be used with intermediates or final products where it is desired to obtain a particular isomer of a compound or to otherwise purify a product of a reaction.

Solvates and/or polymorphs of a compound provided herein or a salt thereof are also contemplated. Solvates contain either stoichiometric or non-stoichiometric amounts of a solvent, and are often formed during the process of crystallization. Hydrates are formed when the solvent is water, or alcoholates are formed when the solvent is alcohol. Polymorphs include the different crystal packing arrangements of the same elemental composition of a compound. Polymorphs usually have different X-ray diffraction patterns, infrared spectra, melting points, density, hardness, crystal shape, optical and electrical properties, stability, and/or solubility. Various factors such as the recrystallization solvent, rate of crystallization, and storage temperature may cause a single crystal form to dominate.

Chromatography, recrystallization and other conventional separation procedures may also be used with intermediates or final products where it is desired to obtain a particular isomer of a compound or to otherwise purify a product of a reaction.

### Enumerated Embodiments

Embodiment 1. A compound of formula (I)

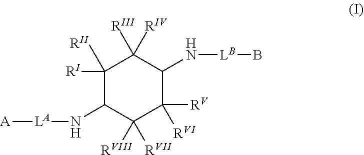

or a pharmaceutically acceptable salt thereof,
wherein:
R^{I}, R^{II}, R^{III}, R^{IV}, R^{V}, R^{VI}, R^{VII}, and R^{VIII}, independently from each other, are selected from the group consisting of hydrogen, C₁-C₆ alkyl, C₁-C₆ haloalkyl, —C(O)OH, —C(O)O(C₁-C₆ alkyl), —C(O)O(C₁-C₆ haloalkyl), and halogen;
or, one of R^{I}, R^{II}, R^{III}, R^{IV}, R^{V}, R^{VI}, R^{VII}, and R^{VIII}, and another one of R^{I}, R^{II}, R^{III}, R^{IV}, R^{V}, R^{VI}, R^{VII}, and R^{VIII}, are taken together to form a C₁-C₆ alkylene moiety;
or, two geminal substituents selected from the group consisting of R^{I}, R^{II}, R^{III}, R^{IV}, R^{V}, R^{VI}, R^{VII}, and R^{VII} are taken together to form an oxo group;
L^{A} is selected from the group consisting

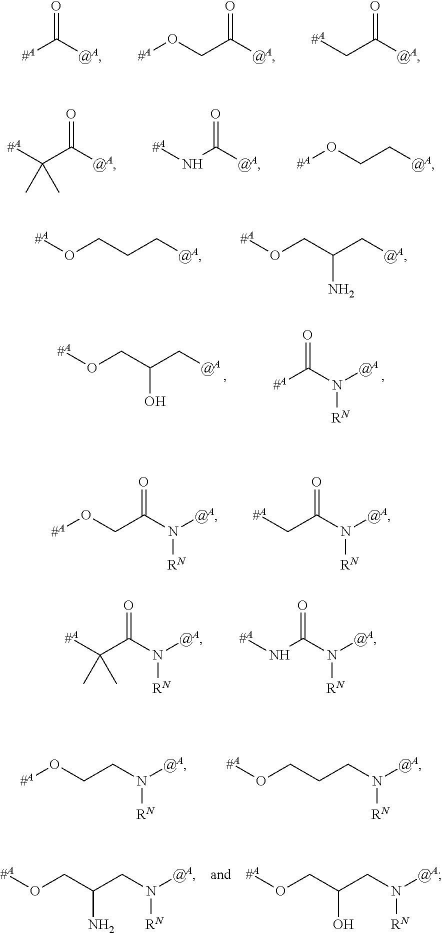

wherein #^{A} represents the attachment point to A and @^{A} represents the attachment point to the remainder of the molecule;
L^{B} is selected from the group consisting of

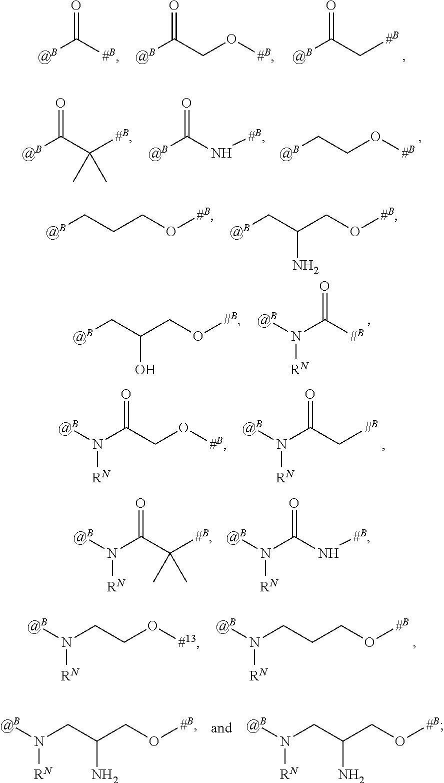

wherein #^{B} represents the attachment point to B and @^{B} represents the attachment point to the remainder of the molecule;
R^{N}, independently at each occurrence, is selected from the group consisting of hydrogen, C₁-C₆ alkyl, and C₁-C₆ haloalkyl,
A is a substituent of formula (A-I)

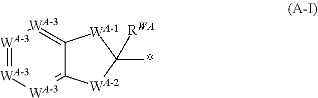

wherein * represents the attachment point to the remainder of the molecule;
   W^{A-1} is selected from the group consisting of —C(R^{WA}-1-R^{WA-1-2}), —N(R^{WA-1-2}—, —C(R^{WA-1-1}R^{WA-1-1})N(R^{WA-1-2})—, —N(R^{WA-1-1})C(R^{WA-1-1}R^{WA-1-2})—, —C(R^{WA-1-1})═N—, —N═C(R^{WA-1-1})—, —O—, —C(R^{WA-1-1}R^{WA-1-1})O—, —OC(R^{WA-1-1}R^{WA-1-2})—, —S—, —C(R^{WA-1-1}R^{WA-1-1})S—, —SC(R^{WA-1-1}R^{WA-1-2})—, —C(R^{WA-1-1}R^{WA-1-1})C(R^{WA-1-1}R^{WA-1-2})—, and —CR^{WA-1-1}═CR^{WA-1-1}—,
      wherein R^{WA-1-1} is H or R^{A}, and R^{WA-1-2} is H or R^{A};
   W^{A-2} is selected from the group consisting of —C(R^{WA-2-1}R^{WA-2-2})—, —N(R^{WA-2-2})—, —C(R^{WA-2-1}R^{WA-2-1})N(R^{WA-2-2})—, —N(R^{WA-2-1})C(R^{WA-2-1}R^{WA-2-2})—, —C(R^{WA-2-1})═N—, —N═C(R^{WA-2-1})—, —O—, —C(R^{WA-2-1}R^{WA-2-1})O—, —OC(R^{WA-2-1}R^{WA-2-2})—, —S—, —C(R^{WA-2-1}R^{WA-2-1})S—, —SC(R^{WA-2-1}R^{WA-2-2})—, —C(R^{WA-2-1}R^{WA-2-1})C(R^{WA-2-1}R^{WA-2-2})—, and —CR^{WA-2-1}═CR^{WA-2-1}—,
      wherein R^{WA-2-1} is H or R^{A}, and R^{WA-2-2} is H or R^{A};
   W^{A-3}, independently at each occurrence, is CR^{WA-3} or N, wherein R^{WA}-3 is H or R^{A};
   R^{WA} is hydrogen or R^{A}, or R^{WA} and R^{WA-1-2} are taken together to form a double bond between the carbon atom bearing R^{WA} and the atom bearing R^{WA-1-2}, or R^{WA} and R^{WA-2-2} are taken together to form a double bond between the carbon atom bearing R^{WA} and the atom bearing R^{WA-2-2};
   C₆-C₁₄ aryl optionally substituted with 1, 2, 3, 4, 5, 6, 7, 8, or 9 R^{A} substituents; and
   5-14 membered heteroaryl optionally substituted with 1, 2, 3, 4, 5, 6, 7, 8, or 9 R^{A} substituents;
      R^{A}, independently at each occurrence, is selected from the group consisting of halogen, NO₂, C₁-C₆ alkyl, C₂-C₆ alkenyl, C₂-C₆ alkynyl, C₁-C₆ haloalkyl, OH, O(C₁-C₆ alkyl), O(C₁-C₆ haloalkyl), SH, S(C₁-C₆ alkyl), S(C₁-C₆ haloalkyl), NH₂, NH(C₁-C₆ alkyl), NH(C₁-C₆ haloalkyl), N(C₁-C₆ alkyl)₂, N(C₁-C₆ haloalkyl)₂, NR^{a}R^{b}, CN, C(O)OH, C(O)O(C₁-C₆ alkyl), C(O)O(C₁-C₆ haloalkyl), C(O)NH₂, C(O)NH(C₁-C₆ alkyl), C(O)NH(C₁-C₆ haloalkyl), C(O)N(C₁-C₆ alkyl)₂, C(O)N(C₁-C₆ haloalkyl)₂, C(O)NR^{a}R^{b}, S(O)₂OH, S(O)₂O(C₁-C₆ alkyl), S(O)₂O(C₁-C₆ haloalkyl), S(O)₂NH₂, S(O)₂NH(C₁-C₆ alkyl), S(O)₂NH(C₁-C₆ haloalkyl), S(O)₂N(C₁-C₆ alkyl)₂, S(O)₂N(C₁-C₆ haloalkyl)₂, S(O)₂NR^{a}R^{b}, OC(O)H, OC(O)(C₁-C₆ alkyl), OC(O)(C₁-C₆ haloalkyl), N(H)C(O)H, N(H)C(O)(C₁-C₆ alkyl), N(H)C(O)(C₁-C₆ haloalkyl), N(C₁-C₆ alkyl)C(O)H, N(C₁-C₆ alkyl)C(O)(C₁-C₆ alkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ alkyl), OS(O)₂(C₁-C₆ haloalkyl), N(H)S(O)₂(C₁-C₆ alkyl), N(H)S(O)₂(C₁-C₆ haloalkyl), N(C₁-C₆ alkyl)S(O)₂(C₁-C₆ alkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl); wherein R^{a} and R^{b} are taken together with the nitrogen atom to which they are attached to form a 3-10 membered heterocycle;
         and
B is selected from the group consisting of:
   a substituent of formula (B-I)

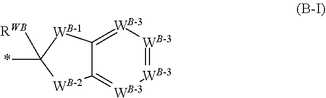

wherein * represents the attachment point to the remainder of the molecule;
   W^{B-1} is selected from the group consisting of —C(R^{WB-1-1}R^{WB-1-2})—, —N(R^{WB-1-2})—, —C(R^{WB-1-1}R^{WB-1-2})N(R^{WB-1-2})—, —N(R^{WB-1-1})C(R^{WB-1-1}R^{WB-1-2})—, —C(R^{WB-1-1})═N—, —N═C(R^{WB-1-1})—, —O—, —C(R^{WB-1-1}R^{WB-1-1})O—, —OC(R^{WB-1-1}R^{WB-1-2})—, —S—, —C(R^{WB-1-1}R^{WB-1-1})S—, —SC(R^{WB-1-1}R^{WB-1-2})—, —C(R^{WB-1-1}R^{WB-1-1})C(R^{WB-1-1}R^{WB-1-2})—, and —CR^{WB-1-1}═CR^{WB-1-1}—, wherein R^{WB-1-1} is H or R^{B}, and R^{WB-1-2} is H or R^{B};
   W^{B-2} is selected from the group consisting of —C(R^{WB-2-1}R^{WB-2-2}), —N(R^{WB-2-2})—, —C(R^{WB-2-1}R^{WB-2-1})N(R^{WB-2-2})—, —N(R^{WB-2-1})C(R^{WB-2-1}R^{WB-2-2})—, —C(R^{WB-2-1})═N—, —N═C(R^{WB-2-1})—, —O—, —C(R^{WB-2-1}R^{WB-2-1})O—, —OC(R^{WB-2-1}R^{WB-2-2})—, —S—, —C(R^{WB-2-1}R^{WB-2-1})S—, —SC(R^{WB-2-1}R^{WB-2-2})—, —C(R^{WB-2-1}R^{WB-2-1})C(R^{WB-2-1}R^{WB-2-2})—, and —CR^{WB-2-1}═CR^{WB-2-1}, wherein R^{WB-2-1} is H or R^{B}, and R^{WB-2-2} is H or R^{B};
   W^{B-3}, independently at each occurrence, is CR^{WB-3} or N, wherein R^{WB-3} is H or R^{B}.
   R^{WB} is hydrogen or R^{B}, or R^{WB} and R^{WB-1-2} are taken together to form a double bond between the carbon atom bearing R^{WB} and the atom bearing R^{WB-1-2}, or R^{WB} and R^{WB-2-2} are taken together to form a double bond between the carbon atom bearing R^{WB} and the atom bearing R^{WB-2-2};
C₆-C₁₄ aryl optionally substituted with 1, 2, 3, 4, 5, 6, 7, 8, or 9 R^{B} substituents; and
5-14 membered heteroaryl optionally substituted with 1, 2, 3, 4, 5, 6, 7, 8, or 9 R^{B} substituents;
   R^{B}, independently at each occurrence, is selected from the group consisting of halogen, NO₂, C₁-C₆ alkyl, C₂-C₆ alkenyl, C₂-C₆ alkynyl, C₁-C₆ haloalkyl, OH, O(C₁-C₆ alkyl), O(C₁-C₆ haloalkyl), SH, S(C₁-C₆ alkyl), S(C₁-C₆ haloalkyl), NH₂, NH(C₁-C₆ alkyl), NH(C₁-C₆ haloalkyl), N(C₁-C₆ alkyl)₂, N(C₁-C₆ haloalkyl)₂, NR^{a}R^{b}, CN, C(O)OH, C(O)O(C₁-C₆ alkyl), C(O)O(C₁-C₆ haloalkyl), C(O)NH₂, C(O)NH(C₁-C₆ alkyl), C(O)NH(C₁-C₆ haloalkyl), C(O)N(C₁-C₆ alkyl)₂, C(O)N(C₁-C₆ haloalkyl)₂, C(O)NR^{a}R^{b}, S(O)₂OH, S(O)₂O(C₁-C₆ alkyl), S(O)₂O(C₁-C₆ haloalkyl), S(O)₂NH₂, S(O)₂NH(C₁-C₆ alkyl), S(O)₂NH(C₁-C₆ haloalkyl), S(O)₂N(C₁-C₆ alkyl)₂, S(O)₂N(C₁-C₆ haloalkyl)₂, S(O)₂NR^{a}R^{b}, OC(O)H, OC(O)(C₁-C₆ alkyl), OC(O)(C₁-C₆ haloalkyl), N(H)C(O)H, N(H)C(O)(C₁-C₆ alkyl), N(H)C(O)(C₁-C₆ haloalkyl), N(C₁-C₆ alkyl)C(O)H, N(C₁-C₆ alkyl)C(O)(C₁-C₆ alkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ alkyl), OS(O)₂(C₁-C₆ haloalkyl), N(H)S(O)₂(C₁-C₆ alkyl), N(H)S(O)₂(C₁-C₆ haloalkyl), N(C₁-C₆ alkyl)S(O)₂(C₁-C₆ alkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl); wherein R^{a} and R^{b} are taken together with the nitrogen atom to which they are attached to form a 3-10 membered heterocycle.

Embodiment 2. A compound of formula (A-1)

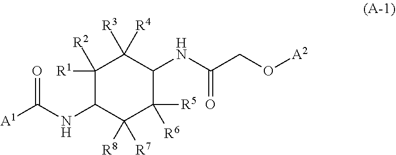

or a pharmaceutically acceptable salt thereof,
wherein:
R¹, R², R³, R⁴, R⁵, R⁶, R⁷, and R⁸, independently from each other, are selected from the group consisting of hydrogen, C₁-C₆ alkyl, C₁-C₆ haloalkyl, —C(O)OH, —C(O)O(C₁-C₆ alkyl), —C(O)O(C₁-C₆ haloalkyl), and halogen;
or, one of R¹, R², R³, R⁴, R⁵, R⁶, R⁷, and R⁸, and another one of R¹, R², R³, R⁴, R⁵, R⁶, R⁷, and R⁸, are taken together to form a C₁-C₆ alkylene moiety;
or, two geminal substituents selected from the group consisting of R¹, R², R³, R⁴, R⁵, R⁶, R⁷, and R⁸ are taken together to form an oxo group;
A¹ is a substituent of formula (A¹-1)

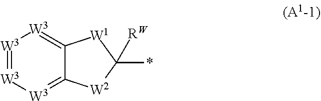

wherein * represents the attachment point to the remainder of the molecule;
   W¹ is selected from the group consisting of —C(R^{W1-1}R^{W1-2})—, —N(R^{W1-2})—, —C(R^{W1-1}R^{W1-1})N(R^{W1-2})—, —N(R^{W1-1})C(R^{W1-1}R^{W1-2})—, —C(R^{W1-1})═N—, —N═C(R^{W1-1})—, —O—, —C(R^{W1-1}R^{W1-1})O—, —OC(R^{W1-1}R^{W1-2})—, —S—, —C(R^{W1-1}R^{W1-1})S—, —SC(R^{W1-1}R^{W1-2})—, —C(R^{W1-1}R^{W1-1})C(R^{W1-1}R^{W1-2})—, and —CR^{W1-1}═CR^{W1-1}—, wherein R^{W1-1} is H or R^{A1}, and R^{W1-2} is H or R^{A1};
W² is selected from the group consisting of —C(R^{W2-1}R^{W2-2})—, —N(R^{W2-2})—, —C(R^{W2-1}R^{W2-1})N(R^{W2-2})—, —N(R^{W2-1})C(R^{W2-1}R^{W2-2})—, —C(R^{W2-1})═N—, —N═C(R^{W2-1})—, —O—, —C(R^{W2-1}R^{W2-1})O—, —OC(R^{W2-1}R^{W2-2})—, —S—, —C(R^{W2-1}R^{W2-1})S, —SC(R^{W2-1}R^{W2-2})—, —C(R^{W2-1}R^{W2-1})C(R^{W2-1}R^{W2-2})—, and —CR^{W2-1}=CR^{W2-1})—, wherein R^{W2-1} is H or R^{A1}, and R^{W2-2} is H or R^{A1};
W³, independently at each occurrence, is CR^{W3} or N, wherein R^{W3} is H or R^{A1};
R^{W} is hydrogen or R^{A}, or R^{W} and R^{W1-2} are taken together to form a double bond between the carbon atom bearing R^{W} and the atom bearing R^{W1-2}, or R^{W} and R^{W2-2} are taken together to form a double bond between the carbon atom bearing R^{W} and the atom bearing R^{W2-2};
R^{A1}, independently at each occurrence, is selected from the group consisting of halogen, NO₂, C₁-C₆ alkyl, C₂-C₆ alkenyl, C₂-C₆ alkynyl, C₁-C₆ haloalkyl, OH, O(C₁-C₆ alkyl), O(C₁-C₆ haloalkyl), SH, S(C₁-C₆ alkyl), S(C₁-C₆ haloalkyl), NH₂, NH(C₁-C₆ alkyl), NH(C₁-C₆ haloalkyl), N(C₁-C₆ alkyl)₂, N(C₁-C₆ haloalkyl)₂, NR^{a}R^{b}, CN, C(O)OH, C(O)O(C₁-C₆ alkyl), C(O)O(C₁-C₆ haloalkyl), C(O)NH₂, C(O)NH(C₁-C₆ alkyl), C(O)NH(C₁-C₆ haloalkyl), C(O)N(C₁-C₆ alkyl)₂, C(O)N(C₁-C₆ haloalkyl)₂, C(O)NR^{a}R^{b}, S(O)₂OH, S(O)₂O(C₁-C₆ alkyl), S(O)₂O(C₁-C₆ haloalkyl), S(O)₂NH₂, S(O)₂NH(C₁-C₆ alkyl), S(O)₂NH(C₁-C₆ haloalkyl), S(O)₂N(C₁-C₆ alkyl)₂, S(O)₂N(C₁-C₆ haloalkyl)₂, S(O)₂NR^{a}R^{b}, OC(O)H, OC(O)(C₁-C₆ alkyl), OC(O)(C₁-C₆ haloalkyl), N(H)C(O)H, N(H)C(O)(C₁-C₆ alkyl), N(H)C(O)(C₁-C₆ haloalkyl), N(C₁-C₆ alkyl)C(O)H, N(C₁-C₆ alkyl)C(O)(C₁-C₆ alkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ alkyl), OS(O)₂(C₁-C₆ haloalkyl), N(H)S(O)₂(C₁-C₆ alkyl), N(H)S(O)₂(C₁-C₆ haloalkyl), N(C₁-C₆ alkyl)S(O)₂(C₁-C₆ alkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl); wherein R^{a} and R^{b} are taken together with the nitrogen atom to which they are attached to form a 3-10 membered heterocycle;
   and
A² is selected from the group consisting of:
   C₆-C₁₄ aryl optionally substituted with 1, 2, 3, 4, 5, 6, 7, 8, or 9 R² substituents; and
   5-14 membered heteroaryl optionally substituted with 1, 2, 3, 4, 5, 6, 7, 8, or 9 R² substituents;
   R^{A2}, independently at each occurrence, is selected from the group consisting of halogen, NO₂, C₁-C₆ alkyl, C₂-C₆ alkenyl, C₂-C₆ alkynyl, C₁-C₆ haloalkyl, OH, O(C₁-C₆ alkyl), O(C₁-C₆ haloalkyl), SH, S(C₁-C₆ alkyl), S(C₁-C₆ haloalkyl), NH₂, NH(C₁-C₆ alkyl), NH(C₁-C₆ haloalkyl), N(C₁-C₆ alkyl)₂, N(C₁-C₆ haloalkyl)₂, NR^{a}R^{b}, CN, C(O)OH, C(O)O(C₁-C₆ alkyl), C(O)O(C₁-C₆ haloalkyl), C(O)NH₂, C(O)NH(C₁-C₆ alkyl), C(O)NH(C₁-C₆ haloalkyl), C(O)N(C₁-C₆ alkyl)₂, C(O)N(C₁-C₆ haloalkyl)₂, C(O)NR^{a}R^{b}, S(O)₂OH, S(O)₂O(C₁-C₆ alkyl), S(O)₂O(C₁-C₆ haloalkyl), S(O)₂NH₂, S(O)₂NH(C₁-C₆ alkyl), S(O)₂NH(C₁-C₆ haloalkyl), S(O)₂N(C₁-C₆ alkyl)₂, S(O)₂N(C₁-C₆ haloalkyl)₂, S(O)₂NR^{a}R^{b}, OC(O)H, OC(O)(C₁-C₆ alkyl), OC(O)(C₁-C₆ haloalkyl), N(H)C(O)H, N(H)C(O)(C₁-C₆ alkyl), N(H)C(O)(C₁-C₆ haloalkyl), N(C₁-C₆ alkyl)C(O)H, N(C₁-C₆ alkyl)C(O)(C₁-C₆ alkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ alkyl), OS(O)₂(C₁-C₆ haloalkyl), N(H)S(O)₂(C₁-C₆ alkyl), N(H)S(O)₂(C₁-C₆ haloalkyl), N(C₁-C₆ alkyl)S(O)₂(C₁-C₆ alkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl); wherein R^{a} and R^{b} are taken together with the nitrogen atom to which they are attached to form a 3-10 membered heterocycle.

Embodiment 3. A compound of formula (B-1)

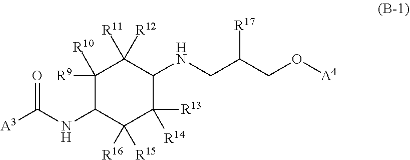

or a pharmaceutically acceptable salt thereof,
wherein:
R⁹, R¹⁰, R¹¹, R¹², R¹³, R¹⁴, R¹⁵, and R¹⁶, independently from each other, are selected from the group consisting of hydrogen, C₁-C₆ alkyl, C₁-C₆ haloalkyl, —C(O)OH, —C(O)O(C₁-C₆ alkyl), —C(O)O(C₁-C₆ haloalkyl), and halogen;
or, one of R⁹, R¹⁰, R¹¹, R¹², R¹³, R¹⁴, R¹⁵, and R¹⁶, and another one of R⁹, R¹⁰, R¹¹, R¹², R¹³, R¹⁴, R¹⁵, and R¹⁶, are taken together to form a C₁-C₆ alkylene moiety;
or, two geminal substituents selected from the group consisting of R⁹, R¹⁰, R¹¹, R¹², R¹³, R¹⁴, R¹⁵, and R¹⁶ are taken together to form an oxo group;
R¹⁷ is H, OH, or NH₂;
A³ is a substituent of formula (A³-1)

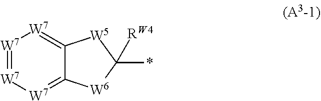

wherein * represents the attachment point to the remainder of the molecule;
   W⁵ is selected from the group consisting of —C(R^{W5-1}R^{W5-2})—, —N(R^{W5-2}), —C(R^{W5-1}R^{W5-2})N(R^{W5-2})—, —N(R^{W5-1})C(R^{W5-1}R^{W5-2})—, —C(R^{W5-1})═N—, —N═C(R^{W5-1}), —O—, —C(R^{W5-1}R^{W5-1})O—, —OC(R^{W5-1}R^{W5-2})—, —S—, —C(R^{W5-1}R^{W5-1})S—, —SC(R^{W5-1}R^{W5-2})—, —C(R^{W5-1}R^{W5-1})C(R^{W5-1}R^{W5-2})—, and —CR^{W5-1}═CR^{W5}—, wherein R^{W5-1} is H or R³, and R^{W5-2} is H or R³;
   W⁶ is selected from the group consisting of —C(R^{W6-1}R^{W6-2})—, —N(R^{W6-2}), —C(R^{W6-1}R^{W6-1})N(R^{W6-2})—, —N(R^{W6-1})C(R^{W6-1}R^{W6-2})—, —C(R^{W6-1})═N—, —N═C(R^{W6-1})—, —O—, —C(R^{W6-1}R^{W6-1})O—, —OC(R^{W6-1}R^{W6-2})—, —S—, —C(R^{W6-1}R^{W6-1})S—, —SC(R^{W6-1}R^{W6-2})—, —C(R^{W6-1}R^{W6-1})C(R^{W6-1}R^{W6-2})—, and —CR^{W6-1}═CR^{W6}—, wherein R^{W6-1} is H or R^{A3}, and R^{W6-2} is H or R³;
   W⁷, independently at each occurrence, is CR^{W7} or N, wherein R^{W7} is H or R^{A3};
   R^{A3}, independently at each occurrence, is selected from the group consisting of halogen, NO₂, C₁-C₆ alkyl, C₂-C₆ alkenyl, C₂-C₆ alkynyl, C₁-C₆ haloalkyl, OH, O(C₁-C₆ alkyl), O(C₁-C₆ haloalkyl), SH, S(C₁-C₆ alkyl), S(C₁-C₆ haloalkyl), NH₂, NH(C₁-C₆ alkyl), NH(C₁-C₆ haloalkyl), N(C₁-C₆ alkyl)₂, N(C₁-C₆ haloalkyl)₂, NR^{a}R^{b}, CN, C(O)OH, C(O)O(C₁-C₆ alkyl), C(O)O(C₁-C₆ haloalkyl), C(O)NH₂, C(O)NH(C₁-C₆ alkyl), C(O)NH(C₁-C₆ haloalkyl), C(O)N(C₁-C₆ alkyl)₂, C(O)N(C₁-C₆ haloalkyl)₂, C(O)NR^{a}R^{b}, S(O)₂OH, S(O)₂O(C₁-C₆ alkyl), S(O)₂O(C₁-C₆ haloalkyl), S(O)₂NH₂, S(O)₂NH(C₁-C₆ alkyl), S(O)₂NH(C₁-C₆ haloalkyl), S(O)₂N(C₁-C₆ alkyl)₂, S(O)₂N(C₁-C₆ haloalkyl)₂, S(O)₂NR^{a}R^{b}, OC(O)H, OC(O)(C₁-C₆ alkyl), OC(O)(C₁-C₆ haloalkyl), N(H)C(O)H, N(H)C(O)(C₁-C₆ alkyl), N(H)C(O)(C₁-C₆ haloalkyl), N(C₁-C₆ alkyl)C(O)H, N(C₁-C₆ alkyl)C(O)(C₁-C₆ alkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ alkyl), OS(O)₂(C₁-C₆ haloalkyl), N(H)S(O)₂(C₁-C₆ alkyl), N(H)S(O)₂(C₁-C₆ haloalkyl), N(C₁-C₆ alkyl)S(O)₂(C₁-C₆ alkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl); wherein R^{a} and R^{b} are taken together with the nitrogen atom to which they are attached to form a 3-10 membered heterocycle;
   R^{W4} is hydrogen or R^{A3}, or R^{W4} and R^{W5-2} are taken together to form a double bond between the carbon atom bearing R^{W4} and the atom bearing R^{W5-2}, or R^{W4} and R^{W6-2} are taken together to form a double bond between the carbon atom bearing R^{W} and the atom bearing R^{W6-2};
      and
A⁴ is selected from the group consisting of:
   C₆-C₁₄ aryl optionally substituted with 1, 2, 3, 4, 5, 6, 7, 8, or 9 R^{A4} substituents; and
   5-14 membered heteroaryl optionally substituted with 1, 2, 3, 4, 5, 6, 7, 8, or 9 R^{A4} substituents;
   R^{A4}, independently at each occurrence, is selected from the group consisting of halogen, NO₂, C₁-C₆ alkyl, C₂-C₆ alkenyl, C₂-C₆ alkynyl, C₁-C₆ haloalkyl, OH, O(C₁-C₆ alkyl), O(C₁-C₆ haloalkyl), SH, S(C₁-C₆ alkyl), S(C₁-C₆ haloalkyl), NH₂, NH(C₁-C₆ alkyl), NH(C₁-C₆ haloalkyl), N(C₁-C₆ alkyl)₂, N(C₁-C₆ haloalkyl)₂, NR^{a}R^{b}, CN, C(O)OH, C(O)O(C₁-C₆ alkyl), C(O)O(C₁-C₆ haloalkyl), C(O)NH₂, C(O)NH(C₁-C₆ alkyl), C(O)NH(C₁-C₆ haloalkyl), C(O)N(C₁-C₆ alkyl)₂, C(O)N(C₁-C₆ haloalkyl)₂, C(O)NR^{a}R^{b}, S(O)₂OH, S(O)₂O(C₁-C₆ alkyl), S(O)₂O(C₁-C₆ haloalkyl), S(O)₂NH₂, S(O)₂NH(C₁-C₆ alkyl), S(O)₂NH(C₁-C₆ haloalkyl), S(O)₂N(C₁-C₆ alkyl)₂, S(O)₂N(C₁-C₆ haloalkyl)₂, S(O)₂NR^{a}R^{b}, OC(O)H, OC(O)(C₁-C₆ alkyl), OC(O)(C₁-C₆ haloalkyl), N(H)C(O)H, N(H)C(O)(C₁-C₆ alkyl), N(H)C(O)(C₁-C₆ haloalkyl), N(C₁-C₆ alkyl)C(O)H, N(C₁-C₆ alkyl)C(O)(C₁-C₆ alkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ alkyl), OS(O)₂(C₁-C₆ haloalkyl), N(H)S(O)₂(C₁-C₆ alkyl), N(H)S(O)₂(C₁-C₆ haloalkyl), N(C₁-C₆ alkyl)S(O)₂(C₁-C₆ alkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl); wherein R^{a} and R^{b} are taken together with the nitrogen atom to which they are attached to form a 3-10 membered heterocycle.

Embodiment 4. A compound of formula (C-1)

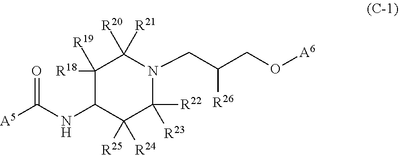

or a pharmaceutically acceptable salt thereof,
wherein:
R¹⁸, R¹⁹, R²⁰, R²¹, R²², R²³, R²⁴, and R², independently from each other, are selected from the group consisting of hydrogen, C₁-C₆ alkyl, C₁-C₆ haloalkyl, —C(O)OH, —C(O)O(C₁-C₆ alkyl), —C(O)O(C₁-C₆ haloalkyl), and halogen;
or, one of R¹⁸, R¹⁹, R²⁰, R²¹, R²², R²³, R²⁴, and R²⁵, and another one of R¹⁸, R¹⁹, R²⁰, R²¹, R²², R²³, R²⁴, and R²⁵, are taken together to form a C₁-C₆ alkylene moiety;
or, two geminal substituents selected from the group consisting of R¹⁸, R¹⁹, R²⁰, R²¹, R²², R²³, R²⁴, and R²⁵ are taken together to form an oxo group;
R²⁶ is H, OH, or NH₂;
A⁵ is a substituent of formula (A⁵-1)

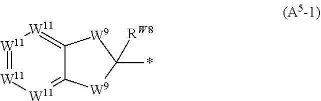

wherein * represents the attachment point to the remainder of the molecule;
   W⁹ is selected from the group consisting of —C(R^{W9-1}R^{W9-2})—, —N(R^{W9-2}), —C(R^{W9-1}R^{W9-2})N(R^{W9-2})—, —N(R^{W9-1})C(R^{W9-1}R^{W9-2})—, —C(R^{W9-1})═N—, —N═C(R^{W9-1})—, —O—, —C(R^{W9-1}R^{W9-1})O—, —OC(R^{W9-1}R^{W9-2})—, —S—, —C(R^{W9-1}R^{W9-1})S—, —SC(R^{W9-1}R^{W9-2})—, —C(R^{W9-1}R^{W9-1})C(R^{W9-1}R^{W9-2})—, and —CR^{W9-1}═CR^{W9-1}, wherein R^{W9-1} is H or R^{A5}, and R^{W9-2} is H or R⁵;
   W¹⁰ is selected from the group consisting of —C(R^{W10-1}R^{W10-2}), —N(R^{W10-2})—, —C(R^{W10-1}R^{W10-1})N(R^{W10-2})—, —N(R^{W10-1})C(R^{W10-1}R^{W10-2})—, —C(R^{W10-1})═N—, —N═C(R^{W10-1})—, —O—, —C(R^{W10-1}R^{W10-1})O—, —OC(R^{W10-1}R^{W10-2})—, —S—, —C(R^{W10-1}R^{W10-1})S—, —SC(R^{W10-1}R^{W10-2})—, —C(R^{W10-1}R^{W10-1})C(R^{W10-1}R^{W10-2})—, and —CR^{W10-1}═CR^{W10-1}—, wherein R^{W10-1} is H or R⁵, and R^{W10-2} is H or R⁵;
   W¹¹, independently at each occurrence, is CR^{W11} or N, wherein R^{W11} is H or R^{A};
   R^{W8} is hydrogen or R^{A5}, or R^{W8} and R^{W9-2} are taken together to form a double bond between the carbon atom bearing R^{W8} and the atom bearing R^{W9-2}, or R^{W8} and R^{W10-2} are taken together to form a double bond between the carbon atom bearing R^{W8} and the atom bearing R^{W10-2};
   R^{A5}, independently at each occurrence, is selected from the group consisting of halogen, NO₂, C₁-C₆ alkyl, C₂-C₆ alkenyl, C₂-C₆ alkynyl, C₁-C₆ haloalkyl, OH, O(C₁-C₆ alkyl), O(C₁-C₆ haloalkyl), SH, S(C₁-C₆ alkyl), S(C₁-C₆ haloalkyl), NH₂, NH(C₁-C₆ alkyl), NH(C₁-C₆ haloalkyl), N(C₁-C₆ alkyl)₂, N(C₁-C₆ haloalkyl)₂, NR^{a}R^{b}, CN, C(O)OH, C(O)O(C₁-C₆ alkyl), C(O)O(C₁-C₆ haloalkyl), C(O)NH₂, C(O)NH(C₁-C₆ alkyl), C(O)NH(C₁-C₆ haloalkyl), C(O)N(C₁-C₆ alkyl)₂, C(O)N(C₁-C₆ haloalkyl)₂, C(O)NR^{a}R^{b}, S(O)₂OH, S(O)₂O(C₁-C₆ alkyl), S(O)₂O(C₁-C₆ haloalkyl), S(O)₂NH₂, S(O)₂NH(C₁-C₆ alkyl), S(O)₂NH(C₁-C₆ haloalkyl), S(O)₂N(C₁-C₆ alkyl)₂, S(O)₂N(C₁-C₆ haloalkyl)₂, S(O)₂NR^{a}R^{b}, OC(O)H, OC(O)(C₁-C₆ alkyl), OC(O)(C₁-C₆ haloalkyl), N(H)C(O)H, N(H)C(O)(C₁-C₆ alkyl), N(H)C(O)(C₁-C₆ haloalkyl), N(C₁-C₆ alkyl)C(O)H, N(C₁-C₆ alkyl)C(O)(C₁-C₆ alkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ alkyl), OS(O)₂(C₁-C₆ haloalkyl), N(H)S(O)₂(C₁-C₆ alkyl), N(H)S(O)₂(C₁-C₆ haloalkyl), N(C₁-C₆ alkyl)S(O)₂(C₁-C₆ alkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl); wherein R^{a} and R^{b} are taken together with the nitrogen atom to which they are attached to form a 3-10 membered heterocycle;
      and
A⁶ is selected from the group consisting of:
   C₆-C₁₄ aryl optionally substituted with 1, 2, 3, 4, 5, 6, 7, 8, or 9 R^{A6} substituents; and
   5-14 membered heteroaryl optionally substituted with 1, 2, 3, 4, 5, 6, 7, 8, or 9 R^{A6} substituents;
   R^{A6}, independently at each occurrence, is selected from the group consisting of halogen, NO₂, C₁-C₆ alkyl, C₂-C₆ alkenyl, C₂-C₆ alkynyl, C₁-C₆ haloalkyl, OH, O(C₁-C₆ alkyl), O(C₁-C₆ haloalkyl), SH, S(C₁-C₆ alkyl), S(C₁-C₆ haloalkyl), NH₂, NH(C₁-C₆ alkyl), NH(C₁-C₆ haloalkyl), N(C₁-C₆ alkyl)₂, N(C₁-C₆ haloalkyl)₂, NR^{a}R^{b}, CN, C(O)OH, C(O)O(C₁-C₆ alkyl), C(O)O(C₁-C₆ haloalkyl), C(O)NH₂, C(O)NH(C₁-C₆ alkyl), C(O)NH(C₁-C₆ haloalkyl), C(O)N(C₁-C₆ alkyl)₂, C(O)N(C₁-C₆ haloalkyl)₂, C(O)NR^{a}R^{b}, S(O)₂OH, S(O)₂O(C₁-C₆ alkyl), S(O)₂O(C₁-C₆ haloalkyl), S(O)₂NH₂, S(O)₂NH(C₁-C₆ alkyl), S(O)₂NH(C₁-C₆ haloalkyl), S(O)₂N(C₁-C₆ alkyl)₂, S(O)₂N(C₁-C₆ haloalkyl)₂, S(O)₂NR^{a}R^{b}, OC(O)H, OC(O)(C₁-C₆ alkyl), OC(O)(C₁-C₆ haloalkyl), N(H)C(O)H, N(H)C(O)(C₁-C₆ alkyl), N(H)C(O)(C₁-C₆ haloalkyl), N(C₁-C₆ alkyl)C(O)H, N(C₁-C₆ alkyl)C(O)(C₁-C₆ alkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ alkyl), OS(O)₂(C₁-C₆ haloalkyl), N(H)S(O)₂(C₁-C₆ alkyl), N(H)S(O)₂(C₁-C₆ haloalkyl), N(C₁-C₆ alkyl)S(O)₂(C₁-C₆ alkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl); wherein R^{a} and R^{b} are taken together with the nitrogen atom to which they are attached to form a 3-10 membered heterocycle.

Embodiment 5. A compound of formula (D-1)

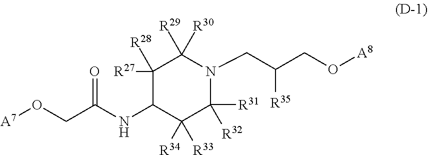

or a pharmaceutically acceptable salt thereof,
wherein:
R²⁷, R²⁸, R²⁹, R³⁰, R³¹, R³², R³³, and R³⁴, independently from each other, are selected from the group consisting of hydrogen, C₁-C₆ alkyl, C₁-C₆ haloalkyl, —C(O)OH, —C(O)O(C₁-C₆ alkyl), —C(O)O(C₁-C₆ haloalkyl), and halogen;
or, one of R²⁷, R²⁸, R²⁹, R³⁰, R³¹, R³², R³³, and R³⁴, and another one of R²⁷, R²⁸, R²⁹, R³⁰, R³¹, R³², R³³, and R³⁴, are taken together to form a C₁-C₆ alkylene moiety;
or, two geminal substituents selected from the group consisting of R²⁷, R²⁸, R²⁹, R³⁰, R³¹, R³², R³³, and R³⁴ are taken together to form an oxo group;
R³⁵ is H, OH, or NH₂;
A⁷ is selected from the group consisting of:
   C₆-C₁₄ aryl optionally substituted with 1, 2, 3, 4, 5, 6, 7, 8, or 9 R^{A7} substituents; and
   5-14 membered heteroaryl optionally substituted with 1, 2, 3, 4, 5, 6, 7, 8, or 9 R^{A7} substituents;
   R^{A7}, independently at each occurrence, is selected from the group consisting of halogen, NO₂, C₁-C₆ alkyl, C₂-C₆ alkenyl, C₂-C₆ alkynyl, C₁-C₆ haloalkyl, OH, O(C₁-C₆ alkyl), O(C₁-C₆ haloalkyl), SH, S(C₁-C₆ alkyl), S(C₁-C₆ haloalkyl), NH₂, NH(C₁-C₆ alkyl), NH(C₁-C₆ haloalkyl), N(C₁-C₆ alkyl)₂, N(C₁-C₆ haloalkyl)₂, NR^{a}R^{b}, CN, C(O)OH, C(O)O(C₁-C₆ alkyl), C(O)O(C₁-C₆ haloalkyl), C(O)NH₂, C(O)NH(C₁-C₆ alkyl), C(O)NH(C₁-C₆ haloalkyl), C(O)N(C₁-C₆ alkyl)₂, C(O)N(C₁-C₆ haloalkyl)₂, C(O)NR^{a}R^{b}, S(O)₂OH, S(O)₂O(C₁-C₆ alkyl), S(O)₂O(C₁-C₆ haloalkyl), S(O)₂NH₂, S(O)₂NH(C₁-C₆ alkyl), S(O)₂NH(C₁-C₆ haloalkyl), S(O)₂N(C₁-C₆ alkyl)₂, S(O)₂N(C₁-C₆ haloalkyl)₂, S(O)₂NR^{a}R^{b}, OC(O)H, OC(O)(C₁-C₆ alkyl), OC(O)(C₁-C₆ haloalkyl), N(H)C(O)H, N(H)C(O)(C₁-C₆ alkyl), N(H)C(O)(C₁-C₆ haloalkyl), N(C₁-C₆ alkyl)C(O)H, N(C₁-C₆ alkyl)C(O)(C₁-C₆ alkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ alkyl), OS(O)₂(C₁-C₆ haloalkyl), N(H)S(O)₂(C₁-C₆ alkyl), N(H)S(O)₂(C₁-C₆ haloalkyl), N(C₁-C₆ alkyl)S(O)₂(C₁-C₆ alkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl); wherein R^{a} and R^{b} are taken together with the nitrogen atom to which they are attached to form a 3-10 membered heterocycle;
      and
A⁸ is selected from the group consisting of:
   C₆-C₁₄ aryl optionally substituted with 1, 2, 3, 4, 5, 6, 7, 8, or 9 R^{A8} substituents; and
   5-14 membered heteroaryl optionally substituted with 1, 2, 3, 4, 5, 6, 7, 8, or 9 R^{A} substituents;
   R^{A8}, independently at each occurrence, is selected from the group consisting of halogen, NO₂, C₁-C₆ alkyl, C₂-C₆ alkenyl, C₂-C₆ alkynyl, C₁-C₆ haloalkyl, OH, O(C₁-C₆ alkyl), O(C₁-C₆ haloalkyl), SH, S(C₁-C₆ alkyl), S(C₁-C₆ haloalkyl), NH₂, NH(C₁-C₆ alkyl), NH(C₁-C₆ haloalkyl), N(C₁-C₆ alkyl)₂, N(C₁-C₆ haloalkyl)₂, NR^{a}R^{b}, CN, C(O)OH, C(O)O(C₁-C₆ alkyl), C(O)O(C₁-C₆ haloalkyl), C(O)NH₂, C(O)NH(C₁-C₆ alkyl), C(O)NH(C₁-C₆ haloalkyl), C(O)N(C₁-C₆ alkyl)₂, C(O)N(C₁-C₆ haloalkyl)₂, C(O)NR^{a}R^{b}, S(O)₂OH, S(O)₂O(C₁-C₆ alkyl), S(O)₂O(C₁-C₆ haloalkyl), S(O)₂NH₂, S(O)₂NH(C₁-C₆ alkyl), S(O)₂NH(C₁-C₆ haloalkyl), S(O)₂N(C₁-C₆ alkyl)₂, S(O)₂N(C₁-C₆ haloalkyl)₂, S(O)₂NR^{a}R^{b}, OC(O)H, OC(O)(C₁-C₆ alkyl), OC(O)(C₁-C₆ haloalkyl), N(H)C(O)H, N(H)C(O)(C₁-C₆ alkyl), N(H)C(O)(C₁-C₆ haloalkyl), N(C₁-C₆ alkyl)C(O)H, N(C₁-C₆ alkyl)C(O)(C₁-C₆ alkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ alkyl), OS(O)₂(C₁-C₆ haloalkyl), N(H)S(O)₂(C₁-C₆ alkyl), N(H)S(O)₂(C₁-C₆ haloalkyl), N(C₁-C₆ alkyl)S(O)₂(C₁-C₆ alkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl); wherein R^{a} and R^{b} are taken together with the nitrogen atom to which they are attached to form a 3-10 membered heterocycle.

Embodiment 6. A compound of formula (II)

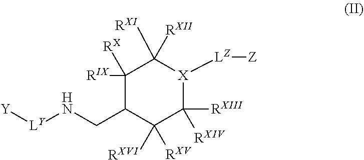

or a pharmaceutically acceptable salt thereof,
wherein:
X is CH or N;
R^{IX}, R^{X}, R^{XI}, R^{XII}, R^{XIII}, R^{IV}, R^{XV}, and R^{XVI}, independently from each other, are selected from the group consisting of hydrogen, C₁-C₆ alkyl, C₁-C₆ haloalkyl, —C(O)OH, —C(O)O(C₁-C₆ alkyl), —C(O)O(C₁-C₆ haloalkyl), and halogen;
or, one of R^{IX}, R^{X}, R^{XI}, R^{XII}, R^{XIII}, R^{IV}, R^{XV}, and R^{XVI}, and another one of R^{IX}, R^{X}, R^{XI}, R^{XI}, R^{XIII}, R^{XIV}, R^{XV}, and R^{XVI}, are taken together to form a C₁-C₆ alkylene moiety;
or, two geminal substituents selected from the group consisting of R^{IX}, R^{X}, R^{XI}, R^{XII}, R^{XIII}, R^{XIV}, R^{XV}, and R^{XVI} are taken together to form an oxo group;
L^{Y} is selected from the group consisting of

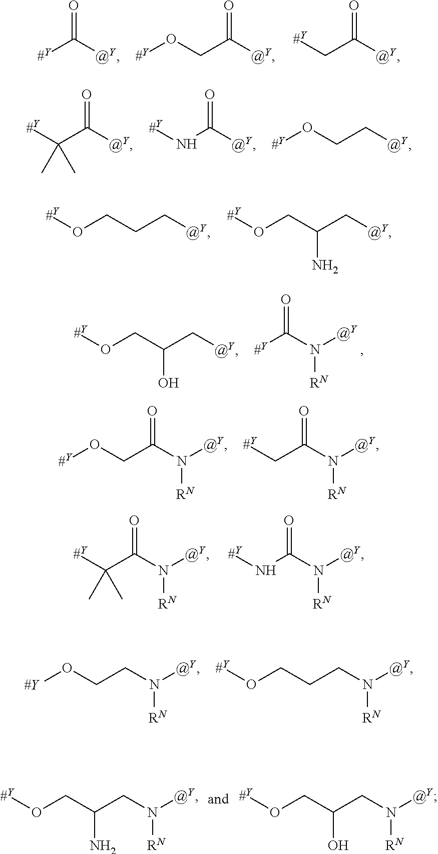

wherein #^{Y} represents the attachment point to Y and @^{Y} represents the attachment point to the remainder of the molecule;
L^{Z} is selected from the group consisting of

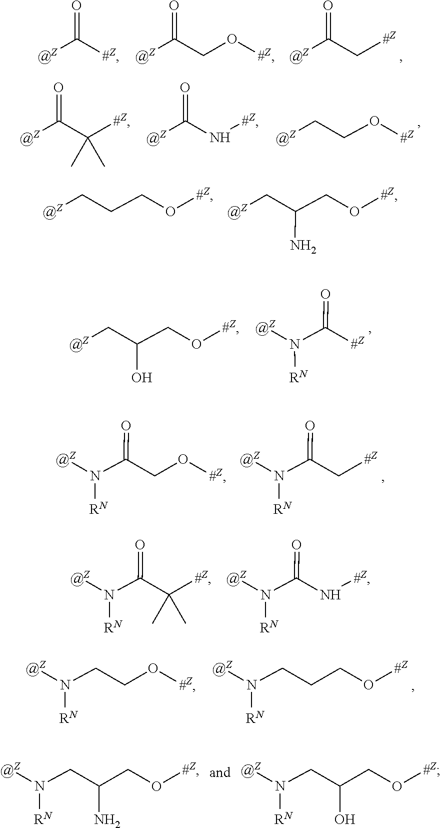

wherein #^{Z} represents the attachment point to Z and @^{Z} represents the attachment point to the remainder of the molecule;
R^{N}, independently at each occurrence, is selected from the group consisting of hydrogen, C₁-C₆ alkyl, and C₁-C₆ haloalkyl,
Y is a substituent of formula (Y-I)

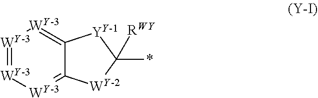

wherein * represents the attachment point to the remainder of the molecule;
   W^{Y-1} is selected from the group consisting of —C(R^{WY-1-1}R^{WY-1-2})—, —N(R^{WY-1-2})—, —C(R^{WY-1-1}R^{WY-1-1})N(R^{WY-1-2})—, —N(R^{WY-1-1})C(R^{WY-1-1}R^{WY-1-2})—, —C(R^{WY-1-1})═N—, —N═C(R^{WY-1-1})—, —O—, —C(R^{WY-1-1}R^{WY-1-1})O—, —OC(R^{WY-1-1}R^{WY-1-2})—, —S, —C(R^{WY-1-1}R^{WY-1-1})S—, —SC(R^{WY-1-1}R^{WY-1-2})—, —C(R^{WY-1-1}R^{WY-1-1})C(R^{WY-1-1}R^{WY-1-2}) and —CR^{WY-1-1}═CR^{WY-1-1}—,
      wherein R^{WY-1-1} is H or R^{Y}, and R^{WY-1-2} is H or R^{Y};
   W^{Y-2} is selected from the group consisting of —C(R^{WY-2-1}R^{WY-2-2})—, —N(R^{WY-2-2})—, —C(R^{WY-2-1}R^{WY-2-1})N(R^{WY-2-2})—, —N(R^{WY-2-1})C(R^{WY-2-1}R^{WY-2-2})—, —C(R^{WY-2-1})═N—, —N═C(R^{WY-2-1})—, —O—, —C(R^{WY-2-1}R^{WY-2-1})O—, —OC(R^{WY-2-1}R^{WY-2-2})—, —S—, —C(R^{WY-2-1}R^{WY-2-1})S—, —SC(R^{WY-2-1}R^{WY-2-2})—, —C(R^{WY-2-1}R^{WY-2-1})C(R^{WY-2-1}R^{WY-2-2}) and —CR^{WY-2-1}═CR^{WY-2-1}—,
      wherein R^{WY-2-1} is H or R^{Y}, and R^{WY-2-2} is H or R^{Y};
   W^{Y-3}, independently at each occurrence, is CR^{WY-3} or N, wherein R^{WY-3} is H or R^{Y};
   R^{WY} is hydrogen or R^{Y}, or R^{WY} and R^{WY-1-2} are taken together to form a double bond between the carbon atom bearing R^{WY} and the atom bearing R^{WY-1-2}, or R^{WY} and R^{WY-2-2} are taken together to form a double bond between the carbon atom bearing R^{WY} and the atom bearing R^{WY-2-2};
   C₆-C₁₄ aryl optionally substituted with 1, 2, 3, 4, 5, 6, 7, 8, or 9 R^{Y} substituents; and
   5-14 membered heteroaryl optionally substituted with 1, 2, 3, 4, 5, 6, 7, 8, or 9 R^{Y} substituents;
      R^{Y}, independently at each occurrence, is selected from the group consisting of halogen, NO₂, C₁-C₆ alkyl, C₂-C₆ alkenyl, C₂-C₆ alkynyl, C₁-C₆ haloalkyl, OH, O(C₁-C₆ alkyl), O(C₁-C₆ haloalkyl), SH, S(C₁-C₆ alkyl), S(C₁-C₆ haloalkyl), NH₂, NH(C₁-C₆ alkyl), NH(C₁-C₆ haloalkyl), N(C₁-C₆ alkyl)₂, N(C₁-C₆ haloalkyl)₂, NR^{a}R^{b}, CN, C(O)OH, C(O)O(C₁-C₆ alkyl), C(O)O(C₁-C₆ haloalkyl), C(O)NH₂, C(O)NH(C₁-C₆ alkyl), C(O)NH(C₁-C₆ haloalkyl), C(O)N(C₁-C₆ alkyl)₂, C(O)N(C₁-C₆ haloalkyl)₂, C(O)NR^{a}R^{b}, S(O)₂OH, S(O)₂O(C₁-C₆ alkyl), S(O)₂O(C₁-C₆ haloalkyl), S(O)₂NH₂, S(O)₂NH(C₁-C₆ alkyl), S(O)₂NH(C₁-C₆ haloalkyl), S(O)₂N(C₁-C₆ alkyl)₂, S(O)₂N(C₁-C₆ haloalkyl)₂, S(O)₂NR^{a}R^{b}, OC(O)H, OC(O)(C₁-C₆ alkyl), OC(O)(C₁-C₆ haloalkyl), N(H)C(O)H, N(H)C(O)(C₁-C₆ alkyl), N(H)C(O)(C₁-C₆ haloalkyl), N(C₁-C₆ alkyl)C(O)H, N(C₁-C₆ alkyl)C(O)(C₁-C₆ alkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ alkyl), OS(O)₂(C₁-C₆ haloalkyl), N(H)S(O)₂(C₁-C₆ alkyl), N(H)S(O)₂(C₁-C₆ haloalkyl), N(C₁-C₆ alkyl)S(O)₂(C₁-C₆ alkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl); wherein R^{a} and R^{b} are taken together with the nitrogen atom to which they are attached to form a 3-10 membered heterocycle;
         and
Z is selected from the group consisting of:
   a substituent of formula (Z-I)

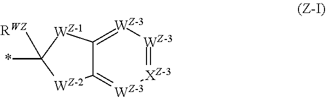

wherein * represents the attachment point to the remainder of the molecule;
   W^{Z-1} is selected from the group consisting of —C(R^{WZ-1-1}R^{WZ-1-2})—, —N(R^{WZ-1-2})—, —C(R^{WZ-1-1}R^{WZ-1-2})N(R^{WZ-1-2})—, —N(R^{WZ-1-1})C(R^{WZ-1-1}R^{WZ-1-2})—, —C(R^{WZ-1-1})═N—, —N═C(R^{WZ-1-1})—, —O—, —C(R^{WZ-1-1}R^{WZ-1-1})O—, —OC(R^{WZ-1-1}R^{WZ-1-2})—, —S—, —C(R^{WZ-1-1}R^{WZ-1-1})S—, —SC(R^{WZ-1-1}R^{WZ-1-2})—, —C(R^{WZ-1-1}R^{WZ-1-1})C(R^{WZ-1-1}R^{WZ-1-2})—, and —CR^{WZ-1-1}═CR^{WZ-1-1},
      wherein R^{WZ-1-1} is H or R^{Z}, and R^{WZ-1-2} is H or R^{Z};
   W^{Z-2} is selected from the group consisting of —C(R^{WZ-2-1}R^{WZ-2-2})—, —N(R^{WZ-2-2})—, —C(R^{WZ-2-1}R^{WZ-2-1})N(R^{WZ-2-2})—, —N(R^{WZ-2-1})C(R^{WZ-2-1}R^{WZ-2-2})—, —C(R^{WZ-2-1})═N—, —N═C(R^{WZ-2-1})—, —O—, —C(R^{WZ-2-1}R^{WZ-2-1})O—, —OC(R^{WZ-2-1}R^{WZ-2-2})—, —S—, —C(R^{WZ-2-1}R^{WZ-2-1})S—, —SC(R^{WZ-2-1}R^{WZ-2-2})—, —C(R^{WZ-2-1}R^{WZ-2-1})C(R^{WZ-2-1}R^{WZ-2-2})—, and —CR^{WZ-2-1}═CR^{WZ-2-1},
      wherein R^{WZ-2-1} is H or R^{Z}, and R^{WZ-2-2} is H or R^{Z};
   W^{Z-3}, independently at each occurrence, is CR^{WZ-3} or N, wherein R^{WZ-3} is H or R^{Z};
   R^{WZ} is hydrogen or R^{Z}, or R^{WZ} and R^{WZ-1-2} are taken together to form a double bond between the carbon atom bearing R^{WZ} and the atom bearing R^{WZ-1-2}, or R^{WZ} and R^{WZ-2-2} are taken together to form a double bond between the carbon atom bearing R^{WZ} and the atom bearing R^{WZ-2-2};
C₆-C₁₄ aryl optionally substituted with 1, 2, 3, 4, 5, 6, 7, 8, or 9 R^{Z} substituents; and
5-14 membered heteroaryl optionally substituted with 1, 2, 3, 4, 5, 6, 7, 8, or 9 R^{Z} substituents;
   R^{Z}, independently at each occurrence, is selected from the group consisting of halogen, NO₂, C₁-C₆ alkyl, C₂-C₆ alkenyl, C₂-C₆ alkynyl, C₁-C₆ haloalkyl, OH, O(C₁-C₆ alkyl), O(C₁-C₆ haloalkyl), SH, S(C₁-C₆ alkyl), S(C₁-C₆ haloalkyl), NH₂, NH(C₁-C₆ alkyl), NH(C₁-C₆ haloalkyl), N(C₁-C₆ alkyl)₂, N(C₁-C₆ haloalkyl)₂, NR^{a}R^{b}, CN, C(O)OH, C(O)O(C₁-C₆ alkyl), C(O)O(C₁-C₆ haloalkyl), C(O)NH₂, C(O)NH(C₁-C₆ alkyl), C(O)NH(C₁-C₆ haloalkyl), C(O)N(C₁-C₆ alkyl)₂, C(O)N(C₁-C₆ haloalkyl)₂, C(O)NR^{a}R^{b}, S(O)₂OH, S(O)₂O(C₁-C₆ alkyl), S(O)₂O(C₁-C₆ haloalkyl), S(O)₂NH₂, S(O)₂NH(C₁-C₆ alkyl), S(O)₂NH(C₁-C₆ haloalkyl), S(O)₂N(C₁-C₆ alkyl)₂, S(O)₂N(C₁-C₆ haloalkyl)₂, S(O)₂NR^{a}R^{b}, OC(O)H, OC(O)(C₁-C₆ alkyl), OC(O)(C₁-C₆ haloalkyl), N(H)C(O)H, N(H)C(O)(C₁-C₆ alkyl), N(H)C(O)(C₁-C₆ haloalkyl), N(C₁-C₆ alkyl)C(O)H, N(C₁-C₆ alkyl)C(O)(C₁-C₆ alkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ alkyl), OS(O)₂(C₁-C₆ haloalkyl), N(H)S(O)₂(C₁-C₆ alkyl), N(H)S(O)₂(C₁-C₆ haloalkyl), N(C₁-C₆ alkyl)S(O)₂(C₁-C₆ alkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl); wherein R^{a} and R^{b} are taken together with the nitrogen atom to which they are attached to form a 3-10 membered heterocycle;
      provided that when L^{Y} is

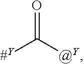

### Y is (Y-I);

when L^{Y} is

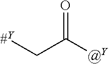

### and L^{Z} is

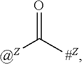

then Y is (Y-I) substituted by 1, 2, 3, 4, 5, 6, 7, 8, or 9 R^{Y} substituents or Z is (Z-I) substituted by 2, 3, 4, 5, 6, 7, 8, or 9 R^{Z} substituents; and
when L^{Y} is

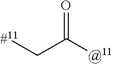

### and L^{Z} is

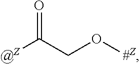

then Y is substituted by 1, 2, 3, 4, 5, 6, 7, 8, or 9 R^{Y} substituents.

Embodiment 7. A compound of formula (E-1)

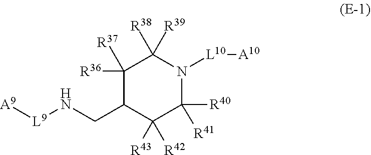

or a pharmaceutically acceptable salt thereof,
wherein:
R³⁶, R³⁷, R³⁸, R³⁹, R⁴⁰, R⁴¹, R⁴², and R⁴³, independently from each other, are selected from the group consisting of hydrogen, C₁-C₆ alkyl, C₁-C₆ haloalkyl, —C(O)OH, —C(O)O(C₁-C₆ alkyl), —C(O)O(C₁-C₆ haloalkyl), and halogen;
or, one of R²⁷, R²⁸, R²⁹, R³⁰, R³¹, R³², R³³, and R³⁴, and another one of R²⁷, R²⁸, R²⁹, R³⁰, R³¹, R³², R³³, and R³⁴, are taken together to form a C₁-C₆ alkylene moiety;
or, two geminal substituents selected from the group consisting of R²⁷, R²⁸, R²⁹, R³⁰, R³¹, R³², R³³, and R³⁴ are taken together to form an oxo group;
L⁹ is selected from the group consisting of a bond,

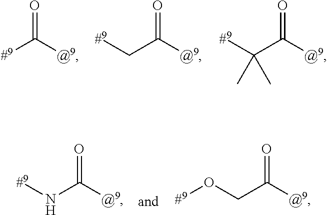

wherein #⁹ represents to attachment point to A⁹ and @⁹ represents the attachment point to the remainder of the molecule;
L¹⁰ is selected from the group consisting of

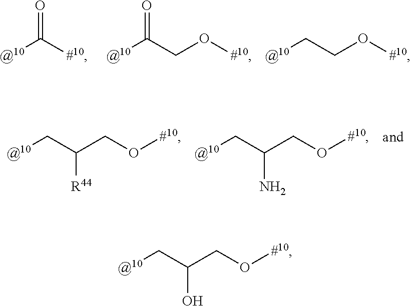

wherein #¹⁰ represents to attachment point to A¹⁰ and @¹⁰ represents the attachment point to the remainder of the molecule;
R⁴⁴ is H, OH, or NH₂;
A⁹ is selected from the group consisting of:
   a substituent of formula (A⁹-1)

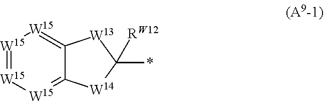

wherein * represents the attachment point to the remainder of the molecule;
   W¹³ is selected from the group consisting of —C(R^{W13-1}R^{W13-2})—, —N(R^{W13-2})—, —C(R^{W13-1}R^{W13-2})N(R^{W13-2})—, —N(R^{W13-1})C(R^{W13-1}R^{W13-2})—, —C(R^{W13-1})═N—, —N═C(R^{W13-1})—, —O—, —C(R^{W13-1}R^{W13-1})O—, —OC(R^{W13-1}R^{W13-2})—, —S—, —C(R^{W13-1}R^{W13-1})S—, —SC(R^{W13-1}R^{W13-2})—, —C(R^{W13-1}R^{W13-1})C(R^{W13-1}R^{W13-2})—, and —CR^{W13-1}═CR^{W13-1}—,
   wherein R^{W13-1} is H or R^{A9}, and R^{W13-2} is H or R^{A};
   W¹⁴ is selected from the group consisting of —C(R^{W14-1}R^{W14-2})—, —N(R^{W14-2})—, —C(R^{W14-1}R^{W14-1})N(R^{W14-2})—, —N(R^{W14-1})C(R^{W14-1}R^{W14-2})—, —C(R^{W14-1})═N—, —N═C(R^{W14-1})—, —O—, —C(R^{W14-1}R^{W14-1})O—, —OC(R^{W14-1}R^{W14-2})—, —S—, —C(R^{W14-1}R^{W14-1})S—, —SC(R^{W14-1}R^{W14-2})—, —C(R^{W14-1}R^{W14-1})C(R^{W14-1}R^{W14-2})—, and —CR^{W14-1}═CR^{W14-1}—,
   wherein R^{W14-1} is H or R^{A9}, and R^{W14-2} is H or R^{A};
   W¹⁵, independently at each occurrence, is CR^{W15} or N, wherein R^{W15} is H or R^{A9};
   R^{W12} is hydrogen or R^{A9}, or R^{W12} and R^{W13-2} are taken together to form a double bond between the carbon atom bearing R^{W12} and the atom bearing R^{W13-2}, or R^{W2} and R^{W14-2} are taken together to form a double bond between the carbon atom bearing R^{W12} and the atom bearing R^{W14-2}.
C₆-C₁₄ aryl optionally substituted with 1, 2, 3, 4, 5, 6, 7, 8, or 9 R^{A9} substituents; and
5-14 membered heteroaryl optionally substituted with 1, 2, 3, 4, 5, 6, 7, 8, or 9 R^{A9} substituents;
R^{A9}, independently at each occurrence, is selected from the group consisting of halogen, NO₂, C₁-C₆ alkyl, C₂-C₆ alkenyl, C₂-C₆ alkynyl, C₁-C₆ haloalkyl, OH, O(C₁-C₆ alkyl), O(C₁-C₆ haloalkyl), SH, S(C₁-C₆ alkyl), S(C₁-C₆ haloalkyl), NH₂, NH(C₁-C₆ alkyl), NH(C₁-C₆ haloalkyl), N(C₁-C₆ alkyl)₂, N(C₁-C₆ haloalkyl)₂, NR^{a}R^{b}, CN, C(O)OH, C(O)O(C₁-C₆ alkyl), C(O)O(C₁-C₆ haloalkyl), C(O)NH₂, C(O)NH(C₁-C₆ alkyl), C(O)NH(C₁-C₆ haloalkyl), C(O)N(C₁-C₆ alkyl)₂, C(O)N(C₁-C₆ haloalkyl)₂, C(O)NR^{a}R^{b}, S(O)₂OH, S(O)₂O(C₁-C₆ alkyl), S(O)₂O(C₁-C₆ haloalkyl), S(O)₂NH₂, S(O)₂NH(C₁-C₆ alkyl), S(O)₂NH(C₁-C₆ haloalkyl), S(O)₂N(C₁-C₆ alkyl)₂, S(O)₂N(C₁-C₆ haloalkyl)₂, S(O)₂NR^{a}R^{b}, OC(O)H, OC(O)(C₁-C₆ alkyl), OC(O)(C₁-C₆ haloalkyl), N(H)C(O)H, N(H)C(O)(C₁-C₆ alkyl), N(H)C(O)(C₁-C₆ haloalkyl), N(C₁-C₆ alkyl)C(O)H, N(C₁-C₆ alkyl)C(O)(C₁-C₆ alkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ alkyl), OS(O)₂(C₁-C₆ haloalkyl), N(H)S(O)₂(C₁-C₆ alkyl), N(H)S(O)₂(C₁-C₆ haloalkyl), N(C₁-C₆ alkyl)S(O)₂(C₁-C₆ alkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl); wherein R^{a} and R^{b} are taken together with the nitrogen atom to which they are attached to form a 3-10 membered heterocycle;
   and
A¹⁰ is selected from the group consisting of:
   a substituent of formula (A¹⁰-1)

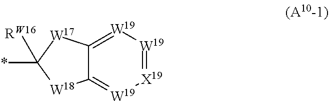

wherein * represents the attachment point to the remainder of the molecule;
   W¹⁷ is selected from the group consisting of —C(R^{W17-1}R^{W17-2})—, —N(R^{W17-2})—, —C(R^{W17-1}R^{W17-2})N(R^{W17-2})—, —N(R^{W17-1})C(R^{W17-1}R^{W17-2})—, —C(R^{W17-1})═N—, —N═C(R^{W17-1})—, —O—, —C(R^{W17-1}R^{W17-1})O—, —OC(R^{W7-1}R^{W17-2})—, —S—, —C(R^{W17-1}R^{W17-1})S—, —SC(R^{W17-1}R^{W17-2})—, —C(R^{W17-1}R^{W17-1})C(R^{W17-1}R^{W17-2})—, and —CR^{W17-1}═CR^{W17}—,
   wherein R^{W17-1} is H or R^{A}, and R^{W17-2} is H or R^{A1};
   W¹⁸ is selected from the group consisting of —C(R^{W18-1}R^{W18-2})—, —N(R^{W18-2})—, —C(R^{W18-1}R^{W18-1})N(R^{W18-2})—, —N(R^{W18-1})C(R^{W18-1}R^{W18-2})—, —C(R^{W18-1})═N—, —N═C(R^{W18-1})—, —O—, —C(R^{W18-1}R^{W18-1})O—, —OC(R^{W8-1}R^{W18-2})—, —S—, —C(R^{W18-1}R^{W18-1})S—, —SC(R^{W18-1}R^{W18-2})—, —C(R^{W18-1}R^{W18-1})C(R^{W18-1}R^{W18-2})—, and —CR^{W18-1}═CR^{W18-1}—,
   wherein R^{W18-1} is H or R^{A}, and R^{W18-2} is H or R^{A1};
   W¹⁹, independently at each occurrence, is CR^{W19} or N, wherein R^{W19} is H or R^{A10};
   R^{W16} is hydrogen or R^{A1}, or R^{W16} and R^{W17-2} are taken together to form a double bond between the carbon atom bearing R^{W16} and the atom bearing R^{W17-2}, or R^{W16} and R^{W18-2} are taken together to form a double bond between the carbon atom bearing R^{W16} and the atom bearing R^{W18-2}.
C₆-C₁₄ aryl optionally substituted with 1, 2, 3, 4, 5, 6, 7, 8, or 9 R^{A10} substituents; and
5-14 membered heteroaryl optionally substituted with 1, 2, 3, 4, 5, 6, 7, 8, or 9 R^{A10} substituents;
R^{A10}, independently at each occurrence, is selected from the group consisting of halogen, NO₂, C₁-C₆ alkyl, C₂-C₆ alkenyl, C₂-C₆ alkynyl, C₁-C₆ haloalkyl, OH, O(C₁-C₆ alkyl), O(C₁-C₆ haloalkyl), SH, S(C₁-C₆ alkyl), S(C₁-C₆ haloalkyl), NH₂, NH(C₁-C₆ alkyl), NH(C₁-C₆ haloalkyl), N(C₁-C₆ alkyl)₂, N(C₁-C₆ haloalkyl)₂, NR^{a}R^{b}, CN, C(O)OH, C(O)O(C₁-C₆ alkyl), C(O)O(C₁-C₆ haloalkyl), C(O)NH₂, C(O)NH(C₁-C₆ alkyl), C(O)NH(C₁-C₆ haloalkyl), C(O)N(C₁-C₆ alkyl)₂, C(O)N(C₁-C₆ haloalkyl)₂, C(O)NR^{a}R^{b}, S(O)₂OH, S(O)₂O(C₁-C₆ alkyl), S(O)₂O(C₁-C₆ haloalkyl), S(O)₂NH₂, S(O)₂NH(C₁-C₆ alkyl), S(O)₂NH(C₁-C₆ haloalkyl), S(O)₂N(C₁-C₆ alkyl)₂, S(O)₂N(C₁-C₆ haloalkyl)₂, S(O)₂NR^{a}R^{b}, OC(O)H, OC(O)(C₁-C₆ alkyl), OC(O)(C₁-C₆ haloalkyl), N(H)C(O)H, N(H)C(O)(C₁-C₆ alkyl), N(H)C(O)(C₁-C₆ haloalkyl), N(C₁-C₆ alkyl)C(O)H, N(C₁-C₆ alkyl)C(O)(C₁-C₆ alkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ alkyl), OS(O)₂(C₁-C₆ haloalkyl), N(H)S(O)₂(C₁-C₆ alkyl), N(H)S(O)₂(C₁-C₆ haloalkyl), N(C₁-C₆ alkyl)S(O)₂(C₁-C₆ alkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl); wherein R^{a} and R^{b} are taken together with the nitrogen atom to which they are attached to form a 3-10 membered heterocycle;
   provided that when L⁹ is

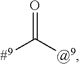

then A⁹ is (A⁹-1).

Embodiment 8. A compound of formula (F-1)

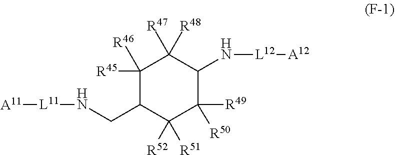

or a pharmaceutically acceptable salt thereof,
wherein:
R⁴⁵, R⁴⁶, R⁴⁷, R⁴⁸, R⁴⁹, R⁵⁰, R⁵¹, and R⁵², independently from each other, are selected from the group consisting of hydrogen, C₁-C₆ alkyl, C₁-C₆ haloalkyl, —C(O)OH, —C(O)O(C₁-C₆ alkyl), —C(O)O(C₁-C₆ haloalkyl), and halogen;
or, one of R⁴⁵, R⁴⁶, R⁴⁷, R⁴⁸, R⁴⁹, R⁵⁰, R⁵¹, and R⁵², and another one of R⁴⁵, R⁴⁶, R⁴⁷, R⁴⁸, R⁴⁹, R⁵⁰, R⁵¹, and R⁵², are taken together to form a C₁-C₆ alkylene moiety;
or, two geminal substituents selected from the group consisting of R⁴⁵, R⁴⁶, R⁴⁷, R⁴⁸, R⁴⁹, R⁵⁰, R⁵¹, and R⁵² are taken together to form an oxo group;
L¹¹ is selected from the group consisting of a bond,

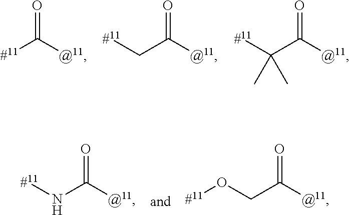

wherein #¹¹ represents to attachment point to A¹¹ and @¹¹ represents the attachment point to the remainder of the molecule;
L¹² is selected from the group consisting of

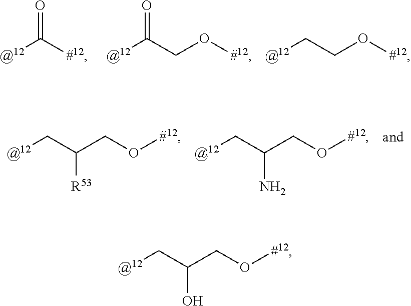

wherein #¹² represents to attachment point to A¹² and @¹² represents the attachment point to the remainder of the molecule;
R⁵³ is H, OH, or NH₂;
A¹¹ is selected from the group consisting of:
   a substituent of formula (A¹¹-1)

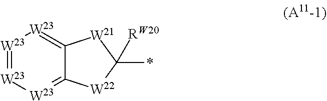

wherein * represents the attachment point to the remainder of the molecule;
   W²¹ is selected from the group consisting of —C(R^{W21-1}R^{W21-2})—, —N(R^{W21-2})—, C(R^{W21-1}R^{W21-2})N(R^{W21-2})—, N(R^{W21-1})C(R^{W21-1}R^{W21-2})—, —C(R^{W21-1})═N—, —N═C(R^{W21-1})—, —O—, —C(R^{W21-1}R^{W21-1})O—, —OC(R^{W21-1}R^{W21-2})—, —S—, —C(R^{W21-1}R^{W21-1})S—, —SC(R^{W21-1}R^{W21-2})—, —C(R^{W21-1}R^{W21-1})C(R^{W21-1}R^{W21-2})—, and —CR^{W21-1}═CR^{W21-1}
   wherein R^{W21-1} is H or R^{A1}, and R^{W21-2} is H or R^{A11};
   W²² is selected from the group consisting of —C(R^{W22-1}R^{W22-2}), —N(R^{W22-2})—, C(R^{W22-1}R^{W22-1})N(R^{W22-2})—, N(R^{W22-1})C(R^{W22-1}R^{W22-2})—, —C(R^{W22-1})═N—, —N═C(R^{W22-1})—, —O—, —C(R^{W22-1}R^{W22-1})O—, —OC(R^{W22-1}R^{W22-2}), —S—, —C(R^{W22-1}R^{W22-1})S—, —SC(R^{W22-1}R^{W22-2})—, —C(R^{W22-1}R^{W22-1})C(R^{W22-1}R^{W22-2})—, and —CR^{W22-1}═CR^{W22-1}—,
   wherein R^{W22-1} is H or R^{A1}, and R^{W22-2} is H or R^{A11};
   W²³, independently at each occurrence, is CR^{W23} or N, wherein R^{W23} is H or R^{A11}.
   R^{W20} is hydrogen or R^{A11}, or R^{W20} and R^{W21-2} are taken together to form a double bond between the carbon atom bearing R^{W20} and the atom bearing R^{W21-2}, or R^{W20} and R^{W22-2} are taken together to form a double bond between the carbon atom bearing R^{W20} and the atom bearing R^{W21-2}.
C₆-C₁₄ aryl optionally substituted with 1, 2, 3, 4, 5, 6, 7, 8, or 9 R^{A11} substituents; and
5-14 membered heteroaryl optionally substituted with 1, 2, 3, 4, 5, 6, 7, 8, or 9 R^{A11} substituents;
R^{A11}, independently at each occurrence, is selected from the group consisting of halogen, NO₂, C₁-C₆ alkyl, C₂-C₆ alkenyl, C₂-C₆ alkynyl, C₁-C₆ haloalkyl, OH, O(C₁-C₆ alkyl), O(C₁-C₆ haloalkyl), SH, S(C₁-C₆ alkyl), S(C₁-C₆ haloalkyl), NH₂, NH(C₁-C₆ alkyl), NH(C₁-C₆ haloalkyl), N(C₁-C₆ alkyl)₂, N(C₁-C₆ haloalkyl)₂, NR^{a}R^{b}, CN, C(O)OH, C(O)O(C₁-C₆ alkyl), C(O)O(C₁-C₆ haloalkyl), C(O)NH₂, C(O)NH(C₁-C₆ alkyl), C(O)NH(C₁-C₆ haloalkyl), C(O)N(C₁-C₆ alkyl)₂, C(O)N(C₁-C₆ haloalkyl)₂, C(O)NR^{a}R^{b}, S(O)₂OH, S(O)₂O(C₁-C₆ alkyl), S(O)₂O(C₁-C₆ haloalkyl), S(O)₂NH₂, S(O)₂NH(C₁-C₆ alkyl), S(O)₂NH(C₁-C₆ haloalkyl), S(O)₂N(C₁-C₆ alkyl)₂, S(O)₂N(C₁-C₆ haloalkyl)₂, S(O)₂NR^{a}R^{b}, OC(O)H, OC(O)(C₁-C₆ alkyl), OC(O)(C₁-C₆ haloalkyl), N(H)C(O)H, N(H)C(O)(C₁-C₆ alkyl), N(H)C(O)(C₁-C₆ haloalkyl), N(C₁-C₆ alkyl)C(O)H, N(C₁-C₆ alkyl)C(O)(C₁-C₆ alkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ alkyl), OS(O)₂(C₁-C₆ haloalkyl), N(H)S(O)₂(C₁-C₆ alkyl), N(H)S(O)₂(C₁-C₆ haloalkyl), N(C₁-C₆ alkyl)S(O)₂(C₁-C₆ alkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl); wherein R^{a} and R^{b} are taken together with the nitrogen atom to which they are attached to form a 3-10 membered heterocycle;
   and
A¹² is selected from the group consisting of:
   a substituent of formula (A¹²-1)

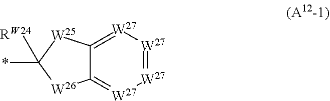

wherein * represents the attachment point to the remainder of the molecule;
   W²⁵ is selected from the group consisting of —C(R^{W25-1}R^{W25-2})—, —N(R^{W25-2})—, —C(R^{W25-1}R^{W25-2})N(R^{W25-2})—, —N(R^{W25-1})C(R^{W25-1}R^{W25-2})—, —C(R^{W25-1})═N—, —N═C(R^{W25-1})—, —O—, —C(R^{W25-1}R^{W25-1})O—, —OC(R^{W25-1}R^{W25-2})—, —S—, —C(R^{W25-1}R^{W25-1})S—, —SC(R^{W25-1}R^{W25-2})—, —C(R^{W25-1}R^{W25-1})C(R^{W25-1}R^{W25-2})—, and —CR^{W25-1}═CR^{W25-1}—,
   wherein R^{W25-1} is H or R^{A12}, and R^{W25-2} is H or R^{A12};
   W²⁶ is selected from the group consisting of —C(R^{W26-1}R^{W26-2})—, —N(R^{W26-2})—, —C(R^{W26-1}R^{W26-1})N(R^{W26-2})—, —N(R^{W26-1})C(R^{W26-1}R^{W26-2})—, —C(R^{W26-1})═N—, —N═C(R^{W26-1})—, —O—, —C(R^{W26-1}R^{W26-1})O—, —OC(R^{W26-1}R^{W26-2})—, —S—, —C(R^{W26-1}R^{W26-1})S—, —SC(R^{W26-1}R^{W26-2})—, —C(R^{W26-1}R^{W26-1})C(R^{W26-1}R^{W26-2})—, and —CR^{W26-1}═CR^{W26-1}—,
   wherein R^{W26-1} is H or R^{A12}, and R^{W26-2} is H or R^{A12}.
   W²⁷, independently at each occurrence, is CR^{W27} or N, wherein R^{W27} is H or R^{A12}.
   R^{W24} is hydrogen or R^{A12}, or R^{W24} and R^{W25-2} are taken together to form a double bond between the carbon atom bearing R^{W24} and the atom bearing R^{W25-2}, or R^{W24} and R^{W26-2} are taken together to form a double bond between the carbon atom bearing R^{W24} and the atom bearing R^{W26-2}.
C₆-C₁₄ aryl optionally substituted with 1, 2, 3, 4, 5, 6, 7, 8, or 9 R^{A12} substituents; and
5-14 membered heteroaryl optionally substituted with 1, 2, 3, 4, 5, 6, 7, 8, or 9 R^{A12} substituents;
R^{A12}, independently at each occurrence, is selected from the group consisting of halogen, NO₂, C₁-C₆ alkyl, C₂-C₆ alkenyl, C₂-C₆ alkynyl, C₁-C₆ haloalkyl, OH, O(C₁-C₆ alkyl), O(C₁-C₆ haloalkyl), SH, S(C₁-C₆ alkyl), S(C₁-C₆ haloalkyl), NH₂, NH(C₁-C₆ alkyl), NH(C₁-C₆ haloalkyl), N(C₁-C₆ alkyl)₂, N(C₁-C₆ haloalkyl)₂, NR^{a}R^{b}, CN, C(O)OH, C(O)O(C₁-C₆ alkyl), C(O)O(C₁-C₆ haloalkyl), C(O)NH₂, C(O)NH(C₁-C₆ alkyl), C(O)NH(C₁-C₆ haloalkyl), C(O)N(C₁-C₆ alkyl)₂, C(O)N(C₁-C₆ haloalkyl)₂, C(O)NR^{a}R^{b}, S(O)₂OH, S(O)₂O(C₁-C₆ alkyl), S(O)₂O(C₁-C₆ haloalkyl), S(O)₂NH₂, S(O)₂NH(C₁-C₆ alkyl), S(O)₂NH(C₁-C₆ haloalkyl), S(O)₂N(C₁-C₆ alkyl)₂, S(O)₂N(C₁-C₆ haloalkyl)₂, S(O)₂NR^{a}R^{b}, OC(O)H, OC(O)(C₁-C₆ alkyl), OC(O)(C₁-C₆ haloalkyl), N(H)C(O)H, N(H)C(O)(C₁-C₆ alkyl), N(H)C(O)(C₁-C₆ haloalkyl), N(C₁-C₆ alkyl)C(O)H, N(C₁-C₆ alkyl)C(O)(C₁-C₆ alkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ alkyl), OS(O)₂(C₁-C₆ haloalkyl), N(H)S(O)₂(C₁-C₆ alkyl), N(H)S(O)₂(C₁-C₆ haloalkyl), N(C₁-C₆ alkyl)S(O)₂(C₁-C₆ alkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl); wherein R^{a} and R^{b} are taken together with the nitrogen atom to which they are attached to form a 3-10 membered heterocycle;
   provided that
when L¹¹ is a bond, then A¹¹ is (A¹¹-1) optionally substituted by 1, 2, 3, 4, 5, 6, 7, 8, or 9 R^{A11} substituents;
when L¹¹ is

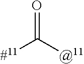

### and L¹² is

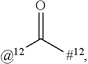

then A¹¹ is (A¹¹-1) substituted by 1, 2, 3, 4, 5, 6, 7, 8, or 9 R^{A11} substituents or A¹² is (A¹¹-1) substituted by 2, 3, 4, 5, 6, 7, 8, or 9 R^{A12} substituents;
and
when L¹¹ is

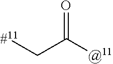

### and L¹² is

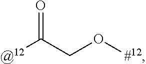

then A¹¹ is substituted by 1, 2, 3, 4, 5, 6, 7, 8, or 9 R^{A11} substituents.

Embodiment 9. A compound of formula (III)

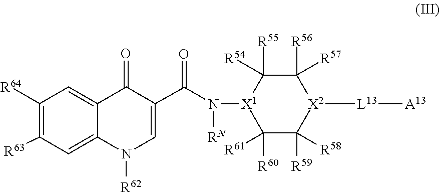

or a salt thereof,
wherein:
X¹ is N or CR^{X1};
X² is N or CR^{X2};
when present, R^{X1} is selected from the group consisting of hydrogen, C₁-C₆ alkyl, C₁-C₆ haloalkyl, —C(O)OH, —C(O)O(C₁-C₆ alkyl), —C(O)O(C₁-C₆ haloalkyl), and halogen;
when present, R^{X2} is selected from the group consisting of hydrogen, C₁-C₆ alkyl, C₁-C₆ haloalkyl, —C(O)OH, —C(O)O(C₁-C₆ alkyl), —C(O)O(C₁-C₆ haloalkyl), and halogen;
R⁵⁴, R⁵⁵, R⁵⁶, R⁵⁷, R⁵⁸, R⁵⁹, R⁶⁰, and R⁶¹, independently from each other, are selected from the group consisting of hydrogen, C₁-C₆ alkyl, C₁-C₆ haloalkyl, —C(O)OH, —C(O)O(C₁-C₆ alkyl), —C(O)O(C₁-C₆ haloalkyl), and halogen;
or, one of R⁵⁴, R⁵⁵, R⁵⁶, R⁵⁷, R⁵⁸, R⁵⁹, R⁶⁰, and R⁶¹, and another one of R⁵⁴, R⁵⁵, R⁵⁶, R⁵⁷, R⁵⁸, R⁵⁹, R⁶⁰, and R⁶¹, are taken together to form a C₁-C₆ alkylene moiety;
or, two geminal substituents selected from the group consisting of R⁵⁴, R⁵⁵, R⁵⁶, R⁵⁷, R⁵⁸, R⁵⁹, R⁶⁰, and R⁶¹ are taken together to form an oxo group;
or, two of R⁵⁴, R⁵⁵, R⁵⁶, R⁵⁷, R⁵⁸, R⁵⁹, R⁶⁰, R⁶¹, R^{X1} when present, and Rx when present, are taken together to form a C₁-C₆ alkylene moiety;
R⁶³ and R⁶⁴, independently from each other, are selected from the group consisting of hydrogen, halogen, NO₂, C₁-C₆ alkyl, C₂-C₆ alkenyl, C₂-C₆ alkynyl, C₁-C₆ haloalkyl, —OH, —O(C₁-C₆ alkyl), —O(C₁-C₆ haloalkyl), —SH, —S(C₁-C₆ alkyl), —S(C₁-C₆ haloalkyl), —NH₂, —NH(C₁-C₆ alkyl), —NH(C₁-C₆ haloalkyl), —N(C₁-C₆ alkyl)₂, —N(C₁-C₆ haloalkyl)₂, —NR^{B-a}R^{B-b}, —CN, —C(O)OH, —C(O)O(C₁-C₆ alkyl), —C(O)O(C₁-C₆ haloalkyl), —C(O)NH₂, —C(O)NH(C₁-C₆ alkyl), —C(O)NH(C₁-C₆ haloalkyl), —C(O)N(C₁-C₆ alkyl)₂, —C(O)N(C₁-C₆ haloalkyl)₂, —C(O)NR^{B-a}R^{B-b}, —S(O)₂OH, —S(O)₂₀(C₁-C₆ alkyl), —S(O)₂₀(C₁-C₆ haloalkyl), —S(O)₂NH₂, —S(O)₂NH(C₁-C₆ alkyl), —S(O)₂NH(C₁-C₆ haloalkyl), —S(O)₂N(C₁-C₆ alkyl)₂, —S(O)₂N(C₁-C₆ haloalkyl)₂, —S(O)₂NR^{B-a}R^{B-b}, —OC(O)H, —OC(O)(C₁-C₆ alkyl), —OC(O)(C₁-C₆ haloalkyl), —N(H)C(O)H, —N(H)C(O)(C₁-C₆ alkyl), —N(H)C(O)(C₁-C₆ haloalkyl), —N(C₁-C₆ alkyl)C(O)H, —N(C₁-C₆ alkyl)C(O)(C₁-C₆ alkyl), —N(C₁-C₆ alkyl)C(O)(C₁-C₆ haloalkyl), —N(C₁-C₆ haloalkyl)C(O)H, —N(C₁-C₆ haloalkyl)C(O)(C₁-C₆ alkyl), —N(C₁-C₆ haloalkyl)C(O)(C₁-C₆ haloalkyl), —OS(O)₂(C₁-C₆ alkyl), —OS(O)₂(C₁-C₆ haloalkyl), —N(H)S(O)₂(C₁-C₆ alkyl), —N(H)S(O)₂(C₁-C₆ haloalkyl), —N(C₁-C₆ alkyl)S(O)₂(C₁-C₆ alkyl), —N(C₁-C₆ alkyl)S(O)₂(C₁-C₆ haloalkyl), —N(C₁-C₆ haloalkyl)S(O)₂(C₁-C₆ alkyl), and —N(C₁-C₆ haloalkyl)S(O)₂(C₁-C₆ haloalkyl);
   wherein R^{B-a} and R^{B-b} are taken together with the nitrogen atom to which they are attached to form a 3-10 membered heterocycle;
R⁶² is selected from the group consisting of halogen, NO₂, C₁-C₆ alkyl, C₂-C₆ alkenyl, C₂-C₆ alkynyl, —(C₁-C₆ alkylene)-(C₆-C₁₄ aryl optionally substituted with 1, 2, 3, 4, 5, 6, 7, 8, or 9 R^{A13} substituents), —(C₁-C₆ alkylene)-(5-14 membered heteroaryl optionally substituted with 1, 2, 3, 4, 5, 6, 7, 8, or 9 R^{A3} substituents), C₁-C₆ haloalkyl, —OH, —O(C₁-C₆ alkyl), —O(C₁-C₆ haloalkyl), —(C₁-C₆ alkylene)-OH, —(C₁-C₆ alkylene)-O—(C₁-C₆ alkyl), —(C₁-C₆ alkylene)-O—(C₁-C₆ haloalkyl), —SH, —S(C₁-C₆ alkyl), —S(C₁-C₆ haloalkyl), —NH₂, —NH(C₁-C₆ alkyl), —NH(C₁-C₆ haloalkyl), —N(C₁-C₆ alkyl)₂, —N(C₁-C₆ haloalkyl)₂, —CN, —C(O)OH, —C(O)O(C₁-C₆ alkyl), —C(O)O(C₁-C₆ haloalkyl), —C(O)NH₂, —C(O)NH(C₁-C₆ alkyl), —C(O)NH(C₁-C₆ haloalkyl), —C(O)N(C₁-C₆ alkyl)₂, —C(O)N(C₁-C₆ haloalkyl)₂, —C(O)NR^{62-a}R^{62-b}, —S(O)₂H, —S(O)₂(C₁-C₆ alkyl), —S(O)₂₀(C₁-C₆ haloalkyl), —S(O)₂NH₂, —S(O)₂NH(C₁-C₆ alkyl), —S(O)₂NH(C₁-C₆ haloalkyl), —S(O)₂N(C₁-C₆ alkyl)₂, —S(O)₂N(C₁-C₆ haloalkyl)₂, —S(O)₂NR^{62-a}R^{62-b}, —OC(O)H, —OC(O)(C₁-C₆ alkyl), —OC(O)(C₁-C₆ haloalkyl), —N(H)C(O)H, —N(H)C(O)(C₁-C₆ alkyl), —N(H)C(O)(C₁-C₆ haloalkyl), —N(C₁-C₆ alkyl)C(O)H, —N(C₁-C₆ alkyl)C(O)(C₁-C₆ alkyl), —N(C₁-C₆ alkyl)C(O)(C₁-C₆ haloalkyl), —N(C₁-C₆ haloalkyl)C(O)H, —N(C₁-C₆ haloalkyl)C(O)(C₁-C₆ alkyl), —N(C₁-C₆ haloalkyl)C(O)(C₁-C₆ haloalkyl), —OS(O)₂(C₁-C₆ alkyl), —OS(O)₂(C₁-C₆ haloalkyl), —N(H)S(O)₂(C₁-C₆ alkyl), —N(H)S(O)₂(C₁-C₆ haloalkyl), —N(C₁-C₆ alkyl)S(O)₂(C₁-C₆ alkyl), —N(C₁-C₆ alkyl)S(O)₂(C₁-C₆ haloalkyl), —N(C₁-C₆ haloalkyl)S(O)₂(C₁-C₆ alkyl), and —N(C₁-C₆ haloalkyl)S(O)₂(C₁-C₆ haloalkyl);
wherein R^{62-a} and R^{62-b} are taken together with the nitrogen atom to which they are attached to form a 3-10 membered heterocycle;
L¹³ is a linker selected from the group consisting of @¹³—C₁-C₆ alkylene-#¹³, @¹³—NR^{N}—(C₁-C₆ alkylene)-#¹³, @¹³NR^{N}—NR^{N}—(C₁-C₆ alkylene)-#¹³ @13—CH₂—NR^{N}—(C₁-C₆ alkylene)-#¹³, @¹³—CH₂—NR^{N}—NR^{N}—(C₁-C₆ alkylene)-#¹³, @¹³—NR^{N}—(C₁-C₆ alkylene)-O-#¹³, @¹³—NR^{N}—NR^{N}—(C₁-C₆ alkylene)-O-#¹³, @¹³—CH₂—NR^{N}—(C₁-C₆ alkylene)-O-#¹³, @¹³—CH₂—NR^{N}—NR^{N}—(C₁-C₆ alkylene)-O-#¹³, and @¹³—(C₁-C₆ alkylene)-O-#¹³;
   wherein @¹³ represents the attachment point to X² and #¹³ represents the attachment point to A¹³;
   the C₁-C₆ alkylene moiety of each of the @¹³—C₁-C₆ alkylene-#¹³, @¹³—NR^{N}—(C₁-C₆ alkylene)-#¹³, @¹³—NR^{N}—NR^{N}—(C₁-C₆ alkylene)-#¹³, @¹³—CH₂—NR^{N}—(C₁-C₆ alkylene)-#¹³, @¹³CH₂—NR^{N}—NR^{N}—(C₁-C₆ alkylene)-#¹³, @¹³—NR^{N}—(C₁-C₆ alkylene)-O-#¹³, @¹³—NR^{N}—NR^{N}—(C₁-C₆ alkylene)-O-#¹³, @¹³CH₂—NR^{N}—(C₁-C₆ alkylene)-O-#¹³, @¹³—CH₂—NR^{N}—NR^{N}—(C₁-C₆ alkylene)-O-#¹³, and @¹³—(C₁-C₆ alkylene)-O-#¹³ is optionally substituted with 1 to 12 R⁶⁶;
   R^{N}, independently at each occurrence, is selected from the group consisting of hydrogen, C₁-C₆ alkyl, and C₁-C₆ haloalkyl,
   R⁶⁶, independently at each occurrence, is selected from the group consisting of oxo, halogen, C₁-C₆ alkyl, C₂-C₆ alkenyl, C₂-C₆ alkynyl, C₁-C₆ haloalkyl, —OH, —O(C₁-C₆ alkyl), —O(C₁-C₆ haloalkyl), —SH, —S(C₁-C₆ alkyl), —S(C₁-C₆ haloalkyl), —NH₂, —NH(C₁-C₆ alkyl), —NH(C₁-C₆ haloalkyl), —N(C₁-C₆ alkyl)₂, —N(C₁-C₆ haloalkyl)₂, —NR^{B-a}R^{B-b}, —CN, —C(O)OH, —C(O)O(C₁-C₆ alkyl), —C(O)O(C₁-C₆ haloalkyl), —C(O)NH₂, —C(O)NH(C₁-C₆ alkyl), —C(O)NH(C₁-C₆ haloalkyl), —C(O)N(C₁-C₆ alkyl)₂, —C(O)N(C₁-C₆ haloalkyl)₂, —C(O)NR^{B-a}R^{B-b}, —S(O)₂OH, —S(O)₂₀(C₁-C₆ alkyl), —S(O)₂₀(C₁-C₆ haloalkyl), —S(O)₂NH₂, —S(O)₂NH(C₁-C₆ alkyl), —S(O)₂NH(C₁-C₆ haloalkyl), —S(O)₂N(C₁-C₆ alkyl)₂, —S(O)₂N(C₁-C₆ haloalkyl)₂, —S(O)₂NR^{B-a}R^{B-b}, —OC(O)H, —OC(O)(C₁-C₆ alkyl), —OC(O)(C₁-C₆ haloalkyl), —N(H)C(O)H, —N(H)C(O)(C₁-C₆ alkyl), —N(H)C(O)(C₁-C₆ haloalkyl), —N(C₁-C₆ alkyl)C(O)H, —N(C₁-C₆ alkyl)C(O)(C₁-C₆ alkyl), —N(C₁-C₆ alkyl)C(O)(C₁-C₆ haloalkyl), —N(C₁-C₆ haloalkyl)C(O)H, —N(C₁-C₆ haloalkyl)C(O)(C₁-C₆ alkyl), —N(C₁-C₆ haloalkyl)C(O)(C₁-C₆ haloalkyl), —OS(O)₂(C₁-C₆ alkyl), —OS(O)₂(C₁-C₆ haloalkyl), —N(H)S(O)₂(C₁-C₆ alkyl), —N(H)S(O)₂(C₁-C₆ haloalkyl), —N(C₁-C₆ alkyl)S(O)₂(C₁-C₆ alkyl), —N(C₁-C₆ alkyl)S(O)₂(C₁-C₆ haloalkyl), —N(C₁-C₆ haloalkyl)S(O)₂(C₁-C₆ alkyl), and —N(C₁-C₆ haloalkyl)S(O)₂(C₁-C₆ haloalkyl);
A¹³ is selected from the group consisting of:
   a substituent of formula (A¹³-1)

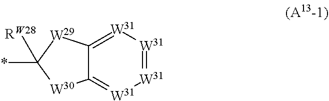

W²⁹ is selected from the group consisting of —C(R^{W29-1}R^{W29-2})—, —N(R^{W29-2})—, —C(R^{W29-1}R^{W29-1})N(R^{W29-2})—, —N(R^{W29-1})C(R^{W29-1}R^{W29-2})—, —C(R^{W29-1})═N—, —N═C(R^{W29-1})—, —O—, —C(R^{W29-1}R^{W29-1})O—, —OC(R^{W29-1}R^{W29-2}), —S—, —C(R^{W29-1}R^{W29-1})S—, —SC(R^{W29-1}R^{W29-2})—, —C(R^{W29-1}R^{W29-1})C(R^{W29-1}R^{W29-2})—, and —CR^{W29-1}═CR^{W29-1}—,
   wherein R^{W29-1} is H or R^{A1}, and R^{W29-2} is H or R^{A13}.
W³⁰ is selected from the group consisting of —C(R^{W30-1}R^{W30-2}), N(R^{W30-2}—, —C(R^{W30-1}R^{W30-1})N(R^{W30-2})—, —N(R^{W30-1})C(R^{W30-1}R^{W30-2})—, —C(R^{W30-1})═N—, —N═C(R^{W30-1})—, —O—, —C(R^{W30-1}R^{W30-1})O—, —OC(R^{W30-1}R^{W30-2}), —S—, —C(R^{W30-1}R^{W30-1})S—, —SC(R^{W30-1}R^{W30-2})—, —C(R^{W30-1}R^{W30-1})C(R^{W30-1}R^{W30-2})—, and —CR^{W30-1}═CR^{W30-1}—,
   wherein R^{W30-1} is H or R^{A13}, and R^{W30-2} is H or R^{A13}.
W³¹, independently at each occurrence, is CR^{W31} or N, wherein R^{W31} is H or R^{A13}.
R^{W28} is hydrogen or R^{A13}, or R^{W28} and R^{W29-2} are taken together to form a double bond between the carbon atom bearing R^{W28} and the atom bearing R^{W29-2}, or R^{W25} and R^{W30-2} are taken together to form a double bond between the carbon atom bearing R^{W28} and the atom bearing R^{W30-2};
C₆-C₁₄ aryl optionally substituted with 1, 2, 3, 4, 5, 6, 7, 8, or 9 R^{A3} substituents; and
5-14 membered heteroaryl optionally substituted with 1, 2, 3, 4, 5, 6, 7, 8, or 9 R^{A3} substituents;
R^{A13}, independently at each occurrence, is selected from the group consisting of halogen, NO₂, C₁-C₆ alkyl, C₂-C₆ alkenyl, C₂-C₆ alkynyl, C₁-C₆ haloalkyl, —OH, —O(C₁-C₆ alkyl), —O(C₁-C₆ haloalkyl), —SH, —S(C₁-C₆ alkyl), —S(C₁-C₆ haloalkyl), —NH₂, —NH(C₁-C₆ alkyl), —NH(C₁-C₆ haloalkyl), —N(C₁-C₆ alkyl)₂, —N(C₁-C₆ haloalkyl)₂, —NR^{A13-a}R^{A13-b}, —CN, —C(O)OH, —C(O)O(C₁-C₆ alkyl), —C(O)O(C₁-C₆ haloalkyl), —C(O)NH₂, —C(O)NH(C₁-C₆ alkyl), —C(O)NH(C₁-C₆ haloalkyl), —C(O)N(C₁-C₆ alkyl)₂, —C(O)N(C₁-C₆ haloalkyl)₂, —C(O)NR^{A13-a}R^{A13-b}, —S(O)₂H, —S(O)₂(C₁-C₆ alkyl), —S(O)₂O(C₁-C₆ haloalkyl), —S(O)₂NH₂, —S(O)₂NH(C₁-C₆ alkyl), —S(O)₂NH(C₁-C₆ haloalkyl), —S(O)₂N(C₁-C₆ alkyl)₂, —S(O)₂N(C₁-C₆ haloalkyl)₂, —S(O)₂NR^{A13-a}R^{A13-b}, —OC(O)H, —OC(O)(C₁-C₆ alkyl), —OC(O)(C₁-C₆ haloalkyl), —N(H)C(O)H, —N(H)C(O)(C₁-C₆ alkyl), —N(H)C(O)(C₁-C₆ haloalkyl), —N(C₁-C₆ alkyl)C(O)H, —N(C₁-C₆ alkyl)C(O)(C₁-C₆ alkyl), —N(C₁-C₆ alkyl)C(O)(C₁-C₆ haloalkyl), —N(C₁-C₆ haloalkyl)C(O)H, —N(C₁-C₆ haloalkyl)C(O)(C₁-C₆ alkyl), —N(C₁-C₆ haloalkyl)C(O)(C₁-C₆ haloalkyl), —OS(O)₂(C₁-C₆ alkyl), —OS(O)₂(C₁-C₆ haloalkyl), —N(H)S(O)₂(C₁-C₆ alkyl), —N(H)S(O)₂(C₁-C₆ haloalkyl), —N(C₁-C₆ alkyl)S(O)₂(C₁-C₆ alkyl), —N(C₁-C₆ alkyl)S(O)₂(C₁-C₆ haloalkyl), —N(C₁-C₆ haloalkyl)S(O)₂(C₁-C₆ alkyl), and —N(C₁-C₆ haloalkyl)S(O)₂(C₁-C₆ haloalkyl);
   wherein R^{A13-a} and R^{A13-b} are taken together with the nitrogen atom to which they are attached to form a 3-10 membered heterocycle;
provided that when X² is N, then L¹³ is a linker selected from the group consisting of @¹³—C₁-C₆ alkylene-#¹³, @¹³—NR^{N}—(C₁-C₆ alkylene)-#¹³, @¹³—NR^{N}—(C₁-C₆ alkylene)-O-#¹³, and @¹³—(C₁-C₆ alkylene)-O-#¹³; and further provided that when X¹ is CH, X² is N, R⁶² is methyl, and L¹³ is @¹³—CH₂-#¹³, then A¹³ is then A¹³ is (A¹³-1), C₆-C₁₄ aryl optionally substituted with 1, 2, 3, 4, 5, 6, 7, 8, or 9 R^{A13} substituents, or 5-14 membered heteroaryl substituted with 1, 2, 3, 4, 5, 6, 7, 8, or 9 R^{A3} substituents.

Embodiment 10. A compound selected from the group consisting of a compound of Table 1, or a pharmaceutically acceptable salt thereof.

Embodiment 11. A compound selected from the group consisting of compounds 1 to 34, or a pharmaceutically acceptable salt thereof.

Embodiment 12. A pharmaceutical composition comprising a compound of any of the preceding embodiments, or a pharmaceutically acceptable salt thereof, and a pharmaceutically acceptable carrier.

Embodiment 13. A method for enhancing protein synthesis in a living organism, comprising administering to the living organism an effective amount of a compound of any one of embodiments 1-11, or a salt thereof.

Embodiment 14. A method for accelerating growth of a plant, comprising administering to the plant an effective amount of a compound of any one of embodiments 1-11, or a salt thereof.

Embodiment 15. A method for improving protein yield or quality in a plant, comprising administering to the plant an effective amount of a compound of any one of embodiments 1-11, or a salt thereof.

Embodiment 16. The method of embodiment 15, wherein the plant is selected from soybean, sunflower, grain legume, rice, wheat germ, maize, tobacco, a cereal, and a lupin crop.

Embodiment 17. A method of treating a disease or disorder mediated by an integrated stress response (ISR) pathway in an individual in need thereof comprising administering to the individual a therapeutically effective amount of a compound of any one of embodiments 1 to 11, or a pharmaceutically acceptable salt thereof, or a therapeutically effective amount of a pharmaceutical composition of embodiment 12.

Embodiment 18. The method of embodiment 17, wherein the compound, the pharmaceutically acceptable salt, or the pharmaceutical composition is administered in combination with a therapeutically effective amount of one or more additional anti-cancer agents.

Embodiment 19. The method of embodiment 17, wherein the disease or disorder is mediated by phosphorylation of eIF2α and/or the guanine nucleotide exchange factor (GEF) activity of eIF2B.

Embodiment 20. The method of any one of embodiments 17-19, wherein the disease or disorder is mediated by a decrease in protein synthesis.

Embodiment 21. The method of any one of embodiments 17-20, wherein the disease or disorder is mediated by the expression of ATF4, CHOP or BACE-1.

Embodiment 22. The method of any of embodiments 17-20, wherein the disease or disorder is a neurodegenerative disease, an inflammatory disease, an autoimmune disease, a metabolic syndrome, a cancer, a vascular disease, an ocular disease, a musculoskeletal disease, or a genetic disorder.

Embodiment 23. The method of embodiment 22, wherein the disease is vanishing white matter disease, childhood ataxia with CNS hypomyelination, intellectual disability syndrome, Alzheimer's disease, prion disease, Creutzfeldt-Jakob disease, Parkinson's disease, amyotrophic lateral sclerosis (ALS) disease, cognitive impairment, frontotemporal dementia (FTD), traumatic brain injury, postoperative cognitive dysfunction (PCD), neuro-otological syndromes, hearing loss, Huntington's disease, stroke, chronic traumatic encephalopathy, spinal cord injury, dementias or cognitive impairment, arthritis, psoriatic arthritis, psoriasis, juvenile idiopathic arthritis, asthma, allergic asthma, bronchial asthma, tuberculosis, chronic airway disorder, cystic fibrosis, glomerulonephritis, membranous nephropathy, sarcoidosis, vasculitis, ichthyosis, transplant rejection, interstitial cystitis, atopic dermatitis or inflammatory bowel disease, Crohn's disease, ulcerative colitis, celiac disease, systemic lupus erythematosus, type 1 diabetes, multiple sclerosis, rheumatoid arthritis, alcoholic liver steatosis, obesity, glucose intolerance, insulin resistance, hyperglycemia, fatty liver, dyslipidemia, hyperlipidemia, type 2 diabetes, pancreatic cancer, breast cancer, kidney cancer, bladder cancer, prostate cancer, testicular cancer, urothelial cancer, endometrial cancer, ovarian cancer, cervical cancer, renal cancer, esophageal cancer, gastrointestinal stromal tumor (GIST), multiple myeloma, cancer of secretory cells, thyroid cancer, gastrointestinal carcinoma, chronic myeloid leukemia, hepatocellular carcinoma, colon cancer, melanoma, malignant glioma, glioblastoma, glioblastoma multiforme, astrocytoma, dysplastic gangliocytoma of the cerebellum, Ewing's sarcoma, rhabdomyosarcoma, ependymoma, medulloblastoma, ductal adenocarcinoma, adenosquamous carcinoma, nephroblastoma, acinar cell carcinoma, lung cancer, non-Hodgkin's lymphoma, Burkitt's lymphoma, chronic lymphocytic leukemia, monoclonal gammopathy of undetermined significance (MGUS), plasmocytoma, lymphoplasmacytic lymphoma, acute lymphoblastic leukemia, Pelizaeus-Merzbacher disease, atherosclerosis, abdominal aortic aneurism, carotid artery disease, deep vein thrombosis, Buerger's disease, chronic venous hypertension, vascular calcification, telangiectasia or lymphoedema, glaucoma, age-related macular degeneration, inflammatory retinal disease, retinal vascular disease, diabetic retinopathy, uveitis, rosacea, Sjogren's syndrome or neovascularization in proliferative retinopathy, hyperhomocysteinemia, skeletal muscle atrophy, myopathy, muscular dystrophy, muscular wasting, sarcopenia, Duchenne muscular dystrophy (DMD), Becker's disease, myotonic dystrophy, X-linked dilated cardiomyopathy, spinal muscular atrophy (SMA), Down syndrome, MEHMO syndrome, metaphyseal chondrodysplasia, Schmid type (MCDS), depression, or social behavior impairment.

Embodiment 24. A method of producing a protein, comprising contacting a eukaryotic cell comprising a nucleic acid encoding the protein with the compound or salt of any one of embodiments 1-11.

Embodiment 25. The method of embodiment 24, comprising culturing the cell in an in vitro culture medium comprising the compound or salt.

Embodiment 26. A method of culturing a eukaryotic cell comprising a nucleic acid encoding a protein, comprising contacting the eukaryotic cell with an in vitro culture medium comprising a compound or salt of any one of embodiments 1-11.

Embodiment 27. The method of any one of embodiments 24-26, wherein the nucleic acid encoding the protein is a recombinant nucleic acid.

Embodiment 28. The method of any one of embodiments 24-27, wherein the cell is a human embryonic kidney (HEK) cell or a Chinese hamster ovary (CHO) cell.

Embodiment 29. The method of any one of embodiments 24-28, wherein the cell is a yeast cell, a wheat germ cell, an insect cell, a rabbit reticulocyte, a cervical cancer cell, a baby hamster kidney cell, a murine myeloma cell, an HT-1080 cell, a PER.C6 cell, a plant cell, a hybridoma cell, or a human blood derived leukocyte

Embodiment 30. A method of producing a protein, comprising contacting a cell-free protein synthesis (CFPS) system comprising eukaryotic initiation factor 2 (eIF2) and a nucleic acid encoding a protein with the compound or salt of any one of embodiments 1-11.

Embodiment 31. The method of anyone of embodiments 24-30, wherein the protein is an antibody or a fragment thereof.

Embodiment 32. The method of anyone of embodiments 24-31, wherein the protein is a recombinant protein, an enzyme, an allergenic peptide, a cytokine, a peptide, a hormone, erythropoietin (EPO), an interferon, a granulocyte-colony stimulating factor (G-CSF), an anticoagulant, or a clotting factor.

Embodiment 33. The method of any one of embodiments 24-32, comprising purifying the protein.

Embodiment 34. An in vitro cell culture medium, comprising the compound or salt of any one of embodiments 1-11 and nutrients for cellular growth.

Embodiment 35. The cell culture medium of embodiment 34, comprising a eukaryotic cell comprising a nucleic acid encoding a protein.

Embodiment 36. The cell culture medium of embodiment 34 or 35, further comprising a compound for inducing protein expression.

Embodiment 37. The cell culture medium of any one of embodiments 34-36, wherein the nucleic acid encoding the protein is a recombinant nucleic acid.

Embodiment 38. The cell culture medium of any one of embodiments 34-37, wherein the protein is an antibody or a fragment thereof.

Embodiment 39. The cell culture medium of any one of embodiments 34-37, wherein the protein is a recombinant protein, an enzyme, an allergenic peptide, a cytokine, a peptide, a hormone, erythropoietin (EPO), an interferon, a granulocyte-colony stimulating factor (G-CSF), an anticoagulant, or a clotting factor.

Embodiment 40. The cell culture medium of any one of embodiments 34-39, wherein the eukaryotic cell is a human embryonic kidney (HEK) cell or a Chinese hamster ovary (CHO) cell.

Embodiment 41. The cell culture medium of anyone of embodiments 34-39, wherein the cell is a yeast cell, a wheat germ cell, an insect cell, a rabbit reticulocyte, a cervical cancer cell, a baby hamster kidney cell, a murine myeloma cell, an HT-1080 cell, a PER.C6 cell, a plant cell, a hybridoma cell, or a human blood derived leukocyte

Embodiment 42. A cell-free protein synthesis (CFPS) system comprising eukaryotic initiation factor 2 (eIF2) and a nucleic acid encoding a protein with the compound or salt of any one of embodiments 1-11.

Embodiment 43. The CFPS system of embodiment 42, comprising a eukaryotic cell extract comprising eIF2.

Embodiment 44. The CFPS system of embodiment 42 or 43, further comprising eIF2B.

Embodiment 45. The CFPS system of any one of embodiments 42-44, wherein the protein is an antibody or a fragment thereof.

Embodiment 46. The CFPS system of any one of embodiments 42-45, wherein the protein is a recombinant protein, an enzyme, an allergenic peptide, a cytokine, a peptide, a hormone, erythropoietin (EPO), an interferon, a granulocyte-colony stimulating factor (G-CSF), an anticoagulant, or a clotting factor.

### EXAMPLES

The chemical reactions in the Examples described can be readily adapted to prepare a number of other compounds disclosed herein, and alternative methods for preparing the compounds of this disclosure are deemed to be within the scope of this disclosure. For example, the synthesis of non-exemplified compounds according to the present disclosure can be successfully performed by modifications apparent to those skilled in the art, e.g., by appropriately protecting interfering groups, by utilizing other suitable reagents known in the art other than those described, or by making routine modifications of reaction conditions, reagents, and starting materials. Alternatively, other reactions disclosed herein or known in the art will be recognized as having applicability for preparing other compounds of the present disclosure.

In some cases, stereoisomers are separated to give single enantiomers or diastereomers as single, unknown stereoisomers, and are arbitrarily drawn as single isomers. Where appropriate, information is given on separation method and elution time and order. In the biological examples, compounds tested were prepared in accordance to the synthetic procedures described therein. For any given compound of unknown absolute stereochemistry for which a stereochemistry has been arbitrarily assigned and for which a specific rotation and/or chiral HPLC elution time has been measured, biological data reported for that compound was obtained using the enantiomer or diastereoisomer associated with said specific rotation and/or chiral HPLC elution time.

In some cases, optical rotation was determined on Jasco DIP-360 digital polarimeter at a wavelength of 589 nm (sodium D line) and are reported as [α]_{D}^{T} for a given temperature T (expressed in ° C.). Where appropriate, information is given on solvent and concentration (expressed as g/100 mL).

Abbreviations:
br. s. Broad singlet
chloroform-d Deuterated chloroform
methanol-d₄ Deuterated methanol
DIAD Diisopropyl azodicarboxylate
DCM Dichloromethane
DEA Diethylamine
DIPEA Diisopropylethylamine
DMF N,N-Dimethylformamide
DMSO-d₆ Deuterated dimethylsulfoxide
d Doublet
EDC.HCl 1-Ethyl-3-(3-dimethylaminopropyl)carbodiimide hydrochloric acid
EtOAc Ethyl acetate
EtOH Ethanol
g Gram
HATU (O-(7-azabenzotriazol-1-yl)-N,N,N′,N′-tetramethyluronium hexafluorophosphate)
HOBT Hydroxybenzotriazole
HPLC High Performance Liquid Chromatography
L Litre
LCMS Liquid Chromatography Mass Spectrometry
MeCN Acetonitrile
MeOH Methanol
mg Milligram
mL Millilitre
mmol Millimoles
m multiplet
NMR Nuclear Magnetic Resonance
iPrOH Isopropanol
q quartet
RT Room temperature
s singlet
SFC Supercritical Fluid Chromatography
TFA trifluoroacetic acid
THF Tetrahydrofuran
TLC Thin layer chromatography
t triplet

### EXAMPLES

### Example 1

### Synthesis of N-(1-(2-amino-3-(4-chloro-3-fluorophenoxy)propyl)piperidin-4-yl)-6-chloroquinoline-2-carboxamide

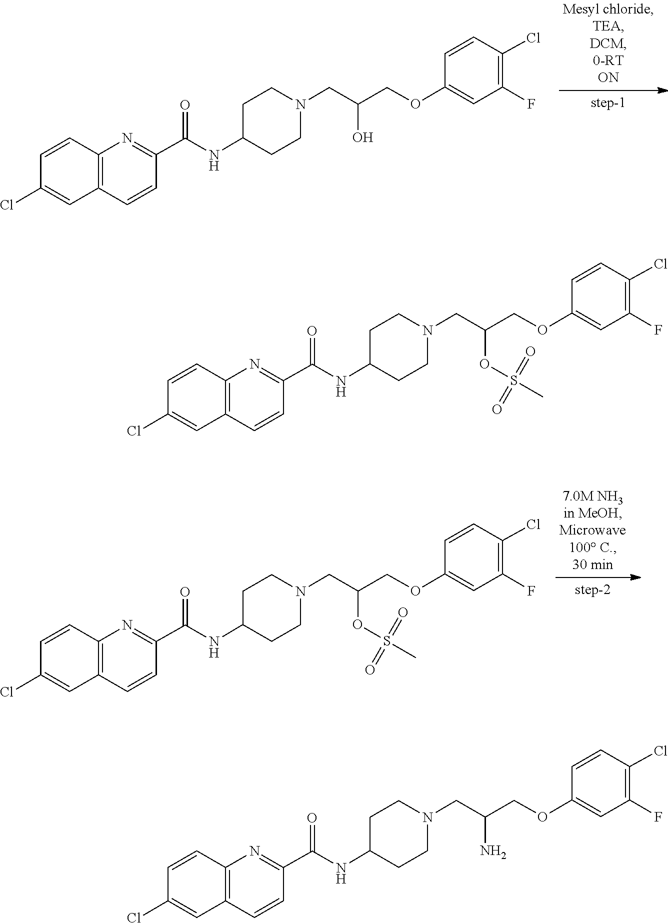 [SMILES: O=C(NC1CCN(CC(O)COc2ccc(Cl)c(F)c2)CC1)c1ccc2cc(Cl)ccc2n1 ; CS(=O)(=O)OC(COc1ccc(Cl)c(F)c1)CN1CCC(NC(=O)c2ccc3cc(Cl)ccc3n2)CC1 ; CS(=O)(=O)OC(COc1ccc(Cl)c(F)c1)CN1CCC(NC(=O)c2ccc3cc(Cl)ccc3n2)CC1 ; NC(COc1ccc(Cl)c(F)c1)CN1CCC(NC(=O)c2ccc3cc(Cl)ccc3n2)CC1]

### Step 1—Synthesis of 1-(4-chloro-3-fluorophenoxy)-3-(4-(6-chloroquinoline-2-carboxamido) piperidin-1-yl)propan-2-yl methanesulfonate

To a stirred solution of 6-chloro-N-(1-(3-(4-chloro-3-fluorophenoxy)-2-hydroxypropyl)piperidin-4-yl)quinoline-2-carboxamide (0.440 g, 0.89 mmol, 1.0 equiv) in DCM (25 mL) was added TEA (2.0 mL) followed by the addition of methanesulfonyl chloride (1.5 mL) at 0° C. After completion of addition the reaction mixture was allowed to stir at RT for overnight. Product formation was confirmed by LCMS. The reaction mixture was diluted with water (50 mL) and extracted with EtOAc (100 mL×2). The combined organic layer was washed with water (50 mL), brine solution (10 mL×2), dried over anhydrous sodium sulfate and concentrated under reduced pressure to obtain 1-(4-chloro-3-fluorophenoxy)-3-(4-(6-chloroquinoline-2-carboxamido)piperidin-1-yl)propan-2-yl methanesulfonate (0.220 g, 43% Yield) as a brown oil. LCMS 570.1 [M+H]⁺.

### Step 2—Synthesis of N-(1-(2-amino-3-(4-chloro-3-fluorophenoxy) propyl) piperidin-4-yl)-6-chloroquinoline-2-carboxamide

1-(4-chloro-3-fluorophenoxy)-3-(4-(6-chloroquinoline-2-carboxamido) piperidin-1-yl)propan-2-yl methanesulfonate (0.220 g, 0.38 mmol, 1.0 equiv) was dissolved in 7.0M Ammonia in MeOH (02 mL). The resultant reaction mixture was heated at 100° C. in microwave for 30 minute. Product formation was confirmed by LCMS. After completion of reaction, the reaction mixture was concentrated under reduced pressure to obtain sticky crude compound which was crystallized in diethyl ether to obtain N-(1-(2-amino-3-(4-chloro-3-fluorophenoxy)propyl)piperidin-4-yl)-6-chloroquinoline-2-carboxamide (Compound 1—0.056 g, 30% Yield) as an off-white solid. LCMS 491.3 [M+H]⁺; ¹H NMR (400 MHz, DMSO-d₆) δ 8.47-8.66 (m, 2H), 8.26 (d, J=2.19 Hz, 1H), 8.06-8.22 (m, 1H), 7.88 (dd, J=9.21, 2.19 Hz, 1H), 7.50 (t, J=8.99 Hz, 1H), 7.14 (d, J=2.63 Hz, 1H), 7.17 (d, J=2.19 Hz, 1H), 6.90 (d, J=8.77 Hz, 1H), 4.18-4.33 (m, 1H), 4.09 (dd, J=10.09, 5.70 Hz, 1H), 3.84 (br. s., 1H), 3.21 (br. s., 1H), 2.97 (d, J=9.65 Hz, 2H), 2.83 (d, J=14.03 Hz, 1H), 2.28-2.44 (m, 2H), 1.86 (br. s., 1H), 1.75 (br. s., 2H).

### Example 2

### Synthesis of 2-(4-chloro-3-fluorophenoxy)-N-(2-(3-(4-chloro-3-fluorophenoxy)-2-hydroxypropyl)-2-azabicyclo[2.2.2]octan-5-yl)acetamide

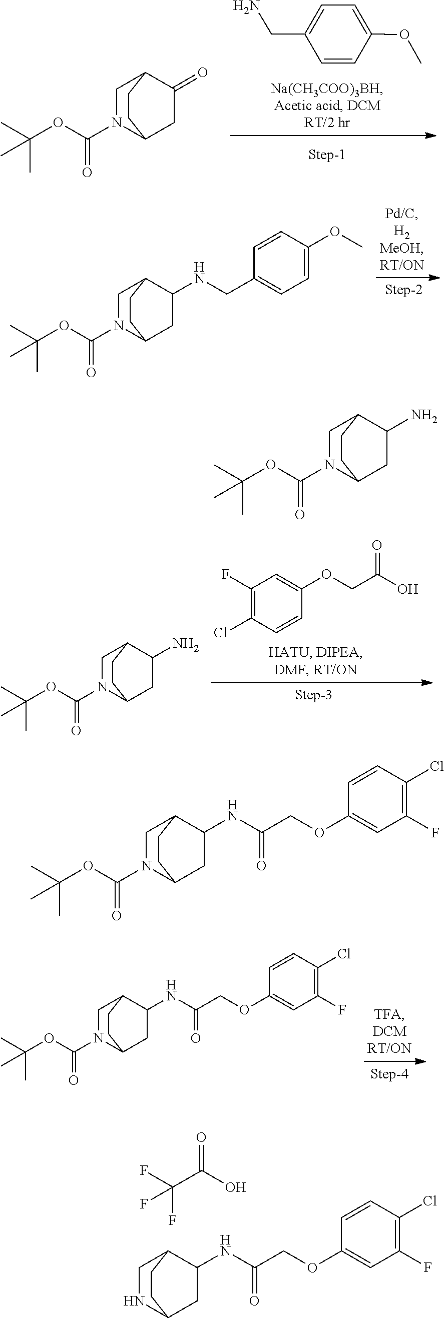

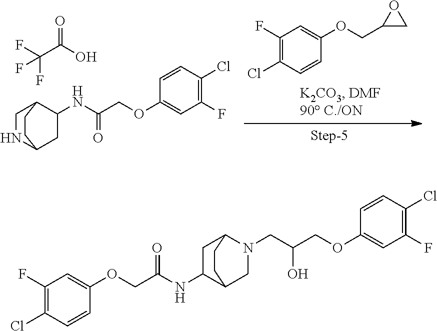

### Step 1—Synthesis of tert-butyl 5-((4-methoxybenzyl)amino)-2-azabicyclo[2.2.2]octane-2-carboxylate

To a solution of tert-butyl 5-oxo-2-azabicyclo[2.2.2]octane-2-carboxylate (2000 mg, 8.8 mmol, 1.0 eq) in DCM (20 mL) was added acetic acid (05 mL), (4-methoxyphenyl)methanamine (1343 mg, 9.7 mmol, 1.1 eq) and sodium triacetoxyborohydride (4850 mg, 22.8 mmol, 2.6 eq) at RT. The reaction mixture was allowed to stir at RT for 2 hr. Product formation was confirmed by LCMS. The reaction mixture was diluted with water (100 mL) and extracted with ethyl acetate (100 mL×2). Combined organic layer was washed with water (100 mL×2), dried over anhydrous Na₂SO₄ and concentrated under reduced pressure to obtain the crude compound which was crystallized in hexane to obtain tert-butyl 5-((4-methoxybenzyl)amino)-2-azabicyclo[2.2.2]octane-2-carboxylate (1500 mg 50% Yield) as a colorless oil. LCMS 347.2 [M+H]⁺.

### Step 2—Synthesis of tert-butyl 5-amino-2-azabicyclo[2.2.2]octane-2-carboxylate

To a stirred solution of tert-butyl 5-((4-methoxybenzyl)amino)-2-azabicyclo[2.2.2]octane-2-carboxylate (150 mg, 0.43 mmol, 1.0 equiv) in Methanol (20 mL) under nitrogen was added Palladium on Carbon[Pd/C](30 mg). Reaction mixture was bubbled with H₂ gas for 16 h. Product formation was confirmed by LCMS. After the completion of reaction, reaction mixture was filtered through Celite® and filtrate was concentrated under reduced pressure to obtain tert-butyl 5-amino-2-azabicyclo[2.2.2]octane-2-carboxylate (100 mg,) as brown semi solid. LCMS 227.2 [M+H]⁺; ¹H NMR (400 MHz, DMSO-d₆) δ 4.01 (br. s., 2H), 3.96 (br. s., 2H), 2.16 (br. s., 1H), 2.06 (d, J=19.73 Hz, 2H), 1.81-1.93 (m, 2H), 1.65-1.81 (m, 2H), 1.54 (br. s., 2H), 1.29-1.47 (m, 9H).

### Step 3—Synthesis of tert-butyl 5-(2-(4-chloro-3-fluorophenoxy)acetamido)-2-azabicyclo[2.2.2]octane-2-carboxylate

To a solution of tert-butyl 5-amino-2-azabicyclo[2.2.2]octane-2-carboxylate (100 mg, 0.44 mmol, 1.0 equiv) in DMF (05 mL) was added 2-(4-chloro-3-fluorophenoxy)acetic acid (90 mg, 0.44 mmol, 1.0 equiv) and HATU (344 mg, 0.88 mmol, 2.0 equiv) at RT. The reaction mixture was stirred for 10 minutes and then DIPEA (0.22 mL, 1.32 mmol, 3.0 equiv) was added. The resultant reaction mixture was allowed to stir at RT for overnight. Progress of the reaction was monitored by LCMS. The reaction mixture was diluted with water (50 mL) and extracted with ethyl acetate (30 mL). Combined organic layer was washed with water (100 mL), dried over anhydrous Na₂SO₄ and concentrated under reduced pressure to obtain the crude compound which was crystallized in hexane to obtain tert-butyl 5-(2-(4-chloro-3-fluorophenoxy)acetamido)-2-azabicyclo[2.2.2]octane-2-carboxylate (100 mg) as an off-white solid. LCMS 413.2 [M+H]⁺.

### Step 4—Synthesis of N-(2-azabicyclo[2.2.2]octan-5-yl)-2-(4-chloro-3-fluorophenoxy)acetamide 2,2,2-trifluoroacetate

To a stirred solution of tert-butyl 5-(2-(4-chloro-3-fluorophenoxy)acetamido)-2-azabicyclo[2.2.2]octane-2-carboxylate (100 mg, 0.24 mmol, 1.0 equiv) in DCM (10 mL) was added TFA (3 mL) at RT. the reaction mixture was allowed to stir at RT overnight. DCM and excess of TFA was removed under reduced pressure to obtain N-(2-azabicyclo[2.2.2]octan-5-yl)-2-(4-chloro-3-fluorophenoxy)acetamide 2,2,2-trifluoroacetate (100 mg, 100% Yield) as a yellow oil. LCMS 313.1 [M+H]⁺.

### Step 5—Synthesis of 2-(4-chloro-3-fluorophenoxy)-N-(2-(3-(4-chloro-3-fluorophenoxy)-2-hydroxypropyl)-2-azabicyclo[2.2.2]octan-5-yl)acetamide

To a stirred solution of N-(2-azabicyclo[2.2.2]octan-5-yl)-2-(4-chloro-3-fluorophenoxy)acetamide 2,2,2-trifluoroacetate (100 mg, 0.24 mmol, 1.0 equiv) in DMF (05 mL) was added K2CO3 (66 mg, 0.48 mmol, 2.0 equiv) followed by the addition of 2-((4-chloro-3-fluorophenoxy)methyl)oxirane (50 mg, 0.24 mmol, 1.0 equiv) at RT. The resulting reaction mixture was heated at 90° C. for overnight. Product formation was confirmed by LCMS. Reaction was stopped by adding water (100 mL) and extracted with EtOAc (100 mL). Combined organic layer was washed with water (2×100 mL), dried over anhydrous sodium sulfate and concentrated under reduced pressure to obtain crude which was purified by flash chromatography (0-5% MeOH in DCM as an eluent) to obtain 2-(4-chloro-3-fluorophenoxy)-N-(2-(3-(4-chloro-3-fluorophenoxy)-2-hydroxypropyl)-2-azabicyclo[2.2.2]octan-5-yl)acetamide (Compound 2—50 mg, 40% Yield) as a brown solid. LCMS 515.2 [M+H]⁺; ¹H NMR (400 MHz, DMSO-d₆) δ 7.59 (d, J=5.26 Hz, 1H), 7.42-7.52 (m, 2H), 7.06 (t, J=2.85 Hz, 1H), 7.09 (t, J=2.85 Hz, 1H), 6.84 (dd, J=1.75, 8.77 Hz, 2H), 4.82 (br. s., 1H), 4.55 (s, 2H), 4.07 (dd, J=3.07, 10.09 Hz, 1H), 3.89-3.95 (m, 1H), 3.79 (br. s., 2H), 3.12 (d, J=18.42 Hz, 2H), 2.34 (d, J=6.58 Hz, 2H), 1.98 (d, J=7.02 Hz, 2H), 1.82 (d, J=7.89 Hz, 2H), 1.71 (d, J=9.65 Hz, 2H), 1.57 (d, J=12.72 Hz, 2H).

### Example 3

### Synthesis of trans-6-chloro-N-(4-(2-(4-chloro-3-fluorophenoxy)acetamido)cyclohexyl)-1H-benzo[d]imidazole-2-carboxamide

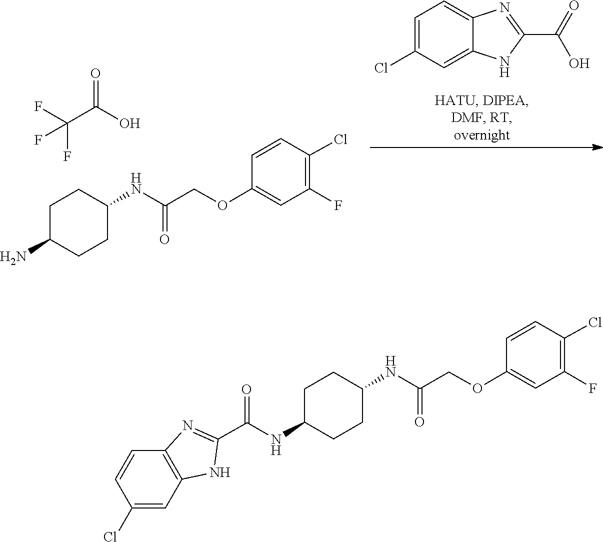

To a stirred solution of 6-chloro-1H-benzo[d]imidazole-2-carboxylic acid (0.100 g, 0.510 mmol, 1.0 equiv) and trans-N-(4-aminocyclohexyl)-2-(4-chloro-3-fluorophenoxy)acetamide 2,2,2-trifluoroacetate (0.153 g, 0.510 mmol, 1.0 equiv) in DMF (5 mL) was added HATU (0.387 g, 1.02 mmol, 2.0 equiv) followed by the addition of DIPEA(0.1 ml, 1.02 mmol, 2.0 equiv) at RT. The resulting reaction mixture was allowed to stir for overnight at RT. Product formation was confirmed by LCMS. After the completion of reaction the reaction mixture was diluted with water (50 mL). The resulting solid was filtered off, washed with water (20 mL×4) and dried under vacuum. The crude product was purified by flash chromatography (0-5% MeOH in DCM as an eluent) to obtain trans-6-chloro-N-(4-(2-(4-chloro-3-fluorophenoxy)acetamido)cyclohexyl)-1H-benzo[d]imidazole-2-carboxamide (Compound 3—0.06 g, 37% Yield) as an off-white solid. LCMS 479.2 [M+H]⁺; ¹H NMR (400 MHz, DMSO-d₆) δ 1.04-1.19 (m, 1H), 1.23 (br. s., 1H), 1.34-1.46 (m, 1H), 1.50-1.66 (m, 2H), 1.82 (br. s., 3H), 1.99 (br. s., 1H), 3.60 (d, J=7.02 Hz, 1H), 3.80 (d, J=9.21 Hz, 1H), 4.36-4.56 (m, 2H), 6.79-6.92 (m, 1H), 7.08 (dd, J=11.40, 2.63 Hz, 1H), 7.32 (dd, J=16.44, 9.87 Hz, 1H), 7.44-7.57 (m, 1H), 7.57-7.64 (m, 1H), 7.73 (d, J=15.79 Hz, 1H), 8.01 (d, J=7.89 Hz, 1H), 8.82 (br. s., 1H).

### Example 4

### Synthesis of 6-chloro-N-(1-(3-(4-chloro-3-fluorophenoxy)-2-hydroxypropyl)piperidin-4-yl)-1H-benzo[d]imidazole-2-carboxamide

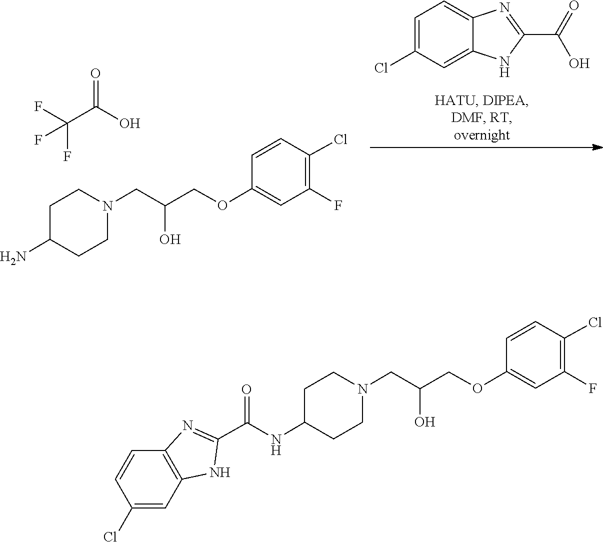

To a stirred solution of 1-(4-aminopiperidin-1-yl)-3-(4-chloro-3-fluorophenoxy)propan-2-ol 2,2,2-trifluoroacetate (0.212 g, 0.50 mmol, 1.0 equiv) in DMF (05 mL) was added HATU (0.290 g, 0.76 mmol, 1.5 equiv) at RT and stirred for 10 minutes. 6-chloro-1H-benzo[d]imidazole-2-carboxylic acid (0.100 g, 0.50 mmol, 1.0 equiv) was added followed by the addition of DIPEA (0.4 mL, 2.04 mmol, 4.0 equiv). The resulting reaction mixture was allowed to stir at RT for overnight. Product formation was confirmed by LCMS. After completion of reaction, the mixture was diluted with water (50 mL) and precipitated solid was filtered off and dried under vacuum to gives crude product which was purified by flash chromatography (0-5% MeOH in DCM as an eluent) to obtain 6-chloro-N-(1-(3-(4-chloro-3-fluorophenoxy)-2-hydroxypropyl)piperidin-4-yl)-1H-benzo[d]imidazole-2-carboxamide (Compound 4—0.060 g, 25% Yield) a white solid. LCMS 481.2 [M+H]⁺; ¹H NMR (400 MHz, DMSO-d₆) δ 13.44 (br. s., 1H), 8.88 (d, J=9.21 Hz, 1H), 7.73 (d, J=8.77 Hz, 1H), 7.45-7.57 (m, 2H), 7.31 (dd, J=16.66, 8.33 Hz, 1H), 7.08 (dd, J=11.84, 2.63 Hz, 1H), 6.86 (dd, J=8.77, 1.75 Hz, 1H), 4.92 (br. s., 1H), 4.01 (d, J=6.58 Hz, 1H), 3.87-3.96 (m, 2H), 3.80 (d, J=8.33 Hz, 1H), 2.85-2.95 (m, 2H), 2.42 (br. s., 1H), 2.35 (d, J=17.54 Hz, 2H), 2.10 (br. s., 2H), 1.72 (br. s., 2H).

### Example 5

### Synthesis of 2-(4-chloro-3-fluorophenoxy)-N-((1-(3-(4-chloro-3-fluorophenoxy)-2-hydroxypropyl)piperidin-4-yl)methyl)acetamide

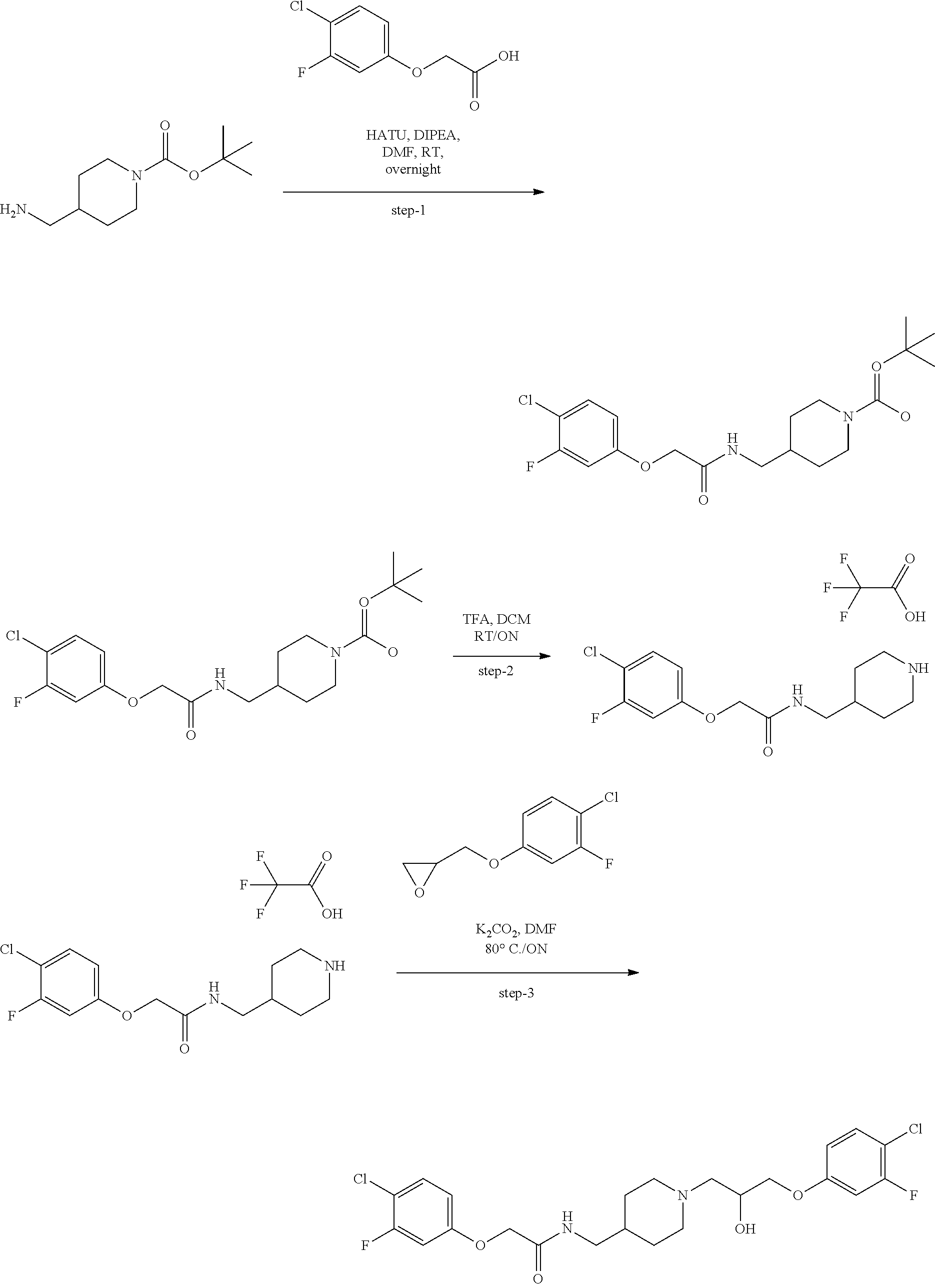

### Step 1—Synthesis of tert-butyl 4-((2-(4-chloro-3-fluorophenoxy)acetamido)methyl)piperidine-1-carboxylate

To a solution of tert-butyl 4-(aminomethyl)piperidine-1-carboxylate (0.500 g, 2.33 mmol, 1.0 equiv) in DMF (10 mL) was added 2-(4-chloro-3-fluorophenoxy)acetic acid (0.476 g, 2.33 mmol, 1.0 equiv) and HATU (1.800 g, 4.66 mmol, 2.0 equiv) at RT. The reaction mixture was stirred for 10 minutes and then DIPEA (1.2 mL, 7.00 mmol, 3.0 equiv) was added. The resultant reaction mixture was allowed to stir at RT for overnight. Product formation was confirmed by 1H NMR. After the completion of reaction the reaction mixture was diluted with water (200 mL). The resulting solid was filtered off, washed with water (100 mL×2) and dried under vacuum to obtain tert-butyl 4-((2-(4-chloro-3-fluorophenoxy)acetamido)methyl)piperidine-1-carboxylate (0.500 g, 53% Yield) as an off-white solid. LCMS 401.1 [M+H]⁺; ¹H NMR (400 MHz, DMSO-d₆) δ 8.09-8.19 (m, 1H), 7.50 (t, J=8.99 Hz, 1H), 7.07 (dd, J=11.40, 3.07 Hz, 1H), 6.78-6.89 (m, 1H), 4.53 (s, 2H), 3.89 (d, J=11.40 Hz, 2H), 3.01 (t, J=6.14 Hz, 2H), 2.56-2.75 (m, 3H), 1.45-1.69 (m, 2H), 1.38 (s, 9H), 0.81-1.06 (m, 2H).

### Step 2—Synthesis of 2-(4-chloro-3-fluorophenoxy)-N-(piperidin-4-ylmethyl)acetamide 2,2,2-trifluoroacetate

To a stirred solution of tert-butyl 4-((2-(4-chloro-3-fluorophenoxy)acetamido)methyl)piperidine-1-carboxylate (0.200 g, 0.500 mmol, 1.0 equiv) in DCM (10 mL) was added TFA (0.2 mL) at RT. The reaction mixture was allowed to stir at RT overnight. Product formation was confirmed by 1H NMR. After the completion of reaction the DCM and excess of TFA was removed under reduced pressure. The crude product was crystallized in diethyl ether to obtain 2-(4-chloro-3-fluorophenoxy)-N-(piperidin-4-ylmethyl)acetamide 2,2,2-trifluoroacetate (0.200 g, 96% Yield) as an off-white solid. LCMS 301.2 [M+H]⁺; ¹H NMR (400 MHz, DMSO-d₆) δ 8.53 (br. s., 1H), 8.25 (t, J=5.92 Hz, 2H), 7.50 (t, J=8.99 Hz, 1H), 7.07 (dd, J=11.40, 3.07 Hz, 1H), 6.85 (dt, J=9.10, 1.37 Hz, 1H), 4.54 (s, 2H), 3.25 (d, J=11.84 Hz, 2H), 3.04 (t, J=6.14 Hz, 2H), 2.75-2.98 (m, 2H), 1.72 (d, J=12.28 Hz, 3H), 1.11-1.33 (m, 2H).

### Step 3—Synthesis of 2-(4-chloro-3-fluorophenoxy)-N-((1-(3-(4-chloro-3-fluorophenoxy)-2-hydroxypropyl)piperidin-4-yl)methyl)acetamide

To a stirred solution of 2-(4-chloro-3-fluorophenoxy)-N-(piperidin-4-ylmethyl)acetamide 2,2,2-trifluoroacetate (0.200 g, 0.483 mmol, 1.0 equiv) in DMF (5 mL) was added K2CO3 (0.134 g, 0.966 mmol, 2.0 equiv) followed by the addition of 2-((4-chloro-3-fluorophenoxy)methyl)oxirane (0.118 g, 0.579 mmol, 1.2 equiv) at RT. The resulting reaction mixture was heated at 80° C. for overnight. Product formation was confirmed by TLC and LCMS. Reaction mixture was diluted with water (50 mL) and extracted with EtOAc (100 mL×2). Combined organic layer was washed with water (4×50 mL), dried over anhydrous sodium sulfate and concentrated under reduced pressure to obtain crude which was purified by flash chromatography (0-5% MeOH in DCM as an eluent) to obtain 2-(4-chloro-3-fluorophenoxy)-N-((1-(3-(4-chloro-3-fluorophenoxy)-2-hydroxypropyl)piperidin-4-yl)methyl)acetamide (Compound 5—0.060 g, 25% Yield) as an off-white solid. LCMS 503.3 [M+H]⁺; ¹H NMR (400 MHz, DMSO-d₆) δ 8.10 (t, J=5.92 Hz, 1H), 7.45 (t, J=8.99 Hz, 1H), 7.49 (t, J=8.77 Hz, 1H), 7.04 (t, J=2.41 Hz, 1H), 7.07 (t, J=2.63 Hz, 1H), 6.78-6.89 (m, 2H), 4.83 (d, J=3.95 Hz, 1H), 4.53 (s, 2H), 3.99 (d, J=7.02 Hz, 1H), 3.79-3.94 (m, 2H), 2.99 (t, J=6.36 Hz, 2H), 2.76-2.94 (m, 2H), 2.17-2.38 (m, 3H), 1.83-2.01 (m, 2H), 1.53 (d, J=12.72 Hz, 2H), 1.39 (br. s., 1H), 0.98-1.17 (m, 2H).

### Example 6

### Synthesis of 5-chloro-N-((1-(3-(4-chloro-3-fluorophenoxy)-2-hydroxypropyl)piperidin-4-yl)methyl)benzofuran-2-carboxamide

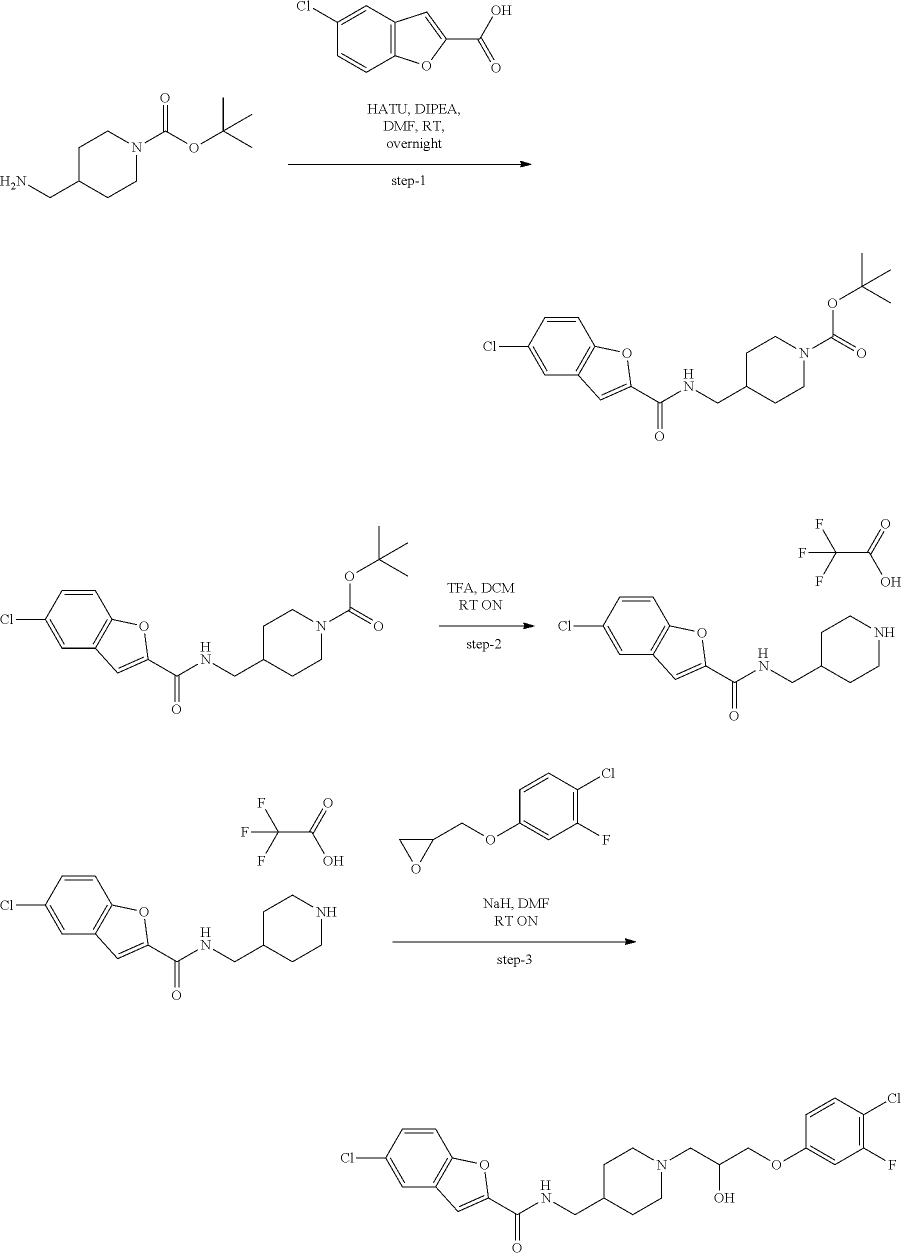

Step 1—Synthesis of tert-butyl 4-((5-chlorobenzofuran-2-carboxamido)methyl)piperidine-1-carboxylate:

To a solution of tert-butyl 4-(ainomnethyl)piperidine-1-carboxylate (0.500 g, 2.33 iniol, 1.0 equiv) in DMF (10 mL) was added 5-chlorobenzofuran-2-carboxylic acid (0.458 g, 2.33 iniol, 1.0 equiv) and HATU (1.800 g, 4.66 ininol, 2.0 equiv) at RT. The reaction mixture was stirred for 10 minutes and then DIPEA (1.2 mL, 7.00 mmol, 3.0 equiv) was added. The resultant reaction mixture was allowed to stir at RT for overnight. Product formation was confirmed by 1H NMR. After the completion of reaction the reaction mixture was diluted with water (200 mL). The resulting solid was filtered off, washed with water (100 mL×2) and dried under vacuum to obtain tert-butyl 4-((5-chlorobenzofuran-2-carboxamido)methyl)piperidine-1-carboxylate (0.300 g, 32% Yield) as an off-white solid. LCMS 393 [M+H]⁺; ¹H NMR (400 MHz, DMSO-d₆) δ 8.83 (t, J=5.70 Hz, 1H), 7.87 (d, J=2.19 Hz, 1H), 7.69 (d, J=9.21 Hz, 1H), 7.39-7.58 (m, 2H), 3.92 (d, J=10.96 Hz, 2H), 3.16 (t, J=6.36 Hz, 2H), 2.89 (br. s., 1H), 2.60-2.76 (m, 2H), 1.54-1.83 (m, 3H), 1.38 (s, 9H), 0.90-1.14 (m, 2H).

### Step 2—Synthesis of 5-chloro-N-(piperidin-4-ylmethyl)benzofuran-2-carboxamide 2,2,2-trifluoroacetate

To a stirred solution tert-butyl 4-((5-chlorobenzofuran-2-carboxamido)methyl)piperidine-1-carboxylate (0.500 g, 1.275 mmol, 1.0 equiv) in DCM (10 mL) was added TFA (1.0 mL) at RT. The reaction mixture was allowed to stir at RT overnight. Product formation was confirmed by 1H NMR. After the completion of reaction the DCM and excess of TFA was removed under reduced pressure. The crude product was crystallized in diethyl ether to 5-chloro-N-(piperidin-4-ylmethyl)benzofuran-2-carboxamide 2,2,2-trifluoroacetate (0.500 g, 96% Yield) as an off-white solid. LCMS 292.9 [M+H]⁺; ¹H NMR (400 MHz, DMSO-d₆) δ 8.92 (br. s., 1H), 8.52 (br. s., 1H), 8.19 (br. s., 1H), 7.88 (s, 1H), 7.70 (d, J=9.21 Hz, 1H), 7.43-7.60 (m, 2H), 3.12-3.33 (m, 4H), 2.75-2.94 (m, 2H), 1.81 (d, J=13.59 Hz, 3H), 1.22-1.42 (m, 2H).

### Step 3—Synthesis of 5-chloro-N-((1-(3-(4-chloro-3-fluorophenoxy)-2-hydroxypropyl)piperidin-4-yl)methyl)benzofuran-2-carboxamide

To a stirred solution of 5-chloro-N-(piperidin-4-ylmethyl)benzofuran-2-carboxamide 2,2,2-trifluoroacetate (0.900 g, 2.21 mmol, 1.0 equiv) in DMF (10 mL) was added NaH (0.265 g, 6.63 mmol, 3.0 equiv) followed by the addition of 2-((4-chloro-3-fluorophenoxy)methyl)oxirane (0.540 g, 2.65 mmol, 1.2 equiv) at RT. The resulting reaction mixture was stir at RT for overnight. Product formation was confirmed by TLC and LCMS. Reaction mixture was diluted with water (50 mL) and extracted with EtOAc (100 mL×2). Combined organic layer was washed with water (4×50 mL), dried over anhydrous sodium sulfate and concentrated under reduced pressure to obtain crude which was purified by flash chromatography (0-5% MeOH in DCM as an eluent) to obtain 5-chloro-N-((1-(3-(4-chloro-3-fluorophenoxy)-2-hydroxypropyl)piperidin-4-yl)methyl)benzofuran-2-carboxamide (Compound 6—0.400 g, 36% Yield) as an off-white solid. LCMS 495.3 [M+H]⁺; ¹H NMR (400 MHz, DMSO-d₆) δ 8.81 (t, J=5.70 Hz, 1H), 7.86 (d, J=1.75 Hz, 1H), 7.69 (d, J=8.77 Hz, 1H), 7.36-7.57 (m, 3H), 7.07 (dd, J=11.40, 2.63 Hz, 1H), 6.83 (dd, J=9.21, 1.75 Hz, 1H), 4.86 (br. s., 1H), 3.94-4.09 (m, 1H), 3.79-3.94 (m, 2H), 3.15 (t, J=6.14 Hz, 2H), 2.87 (d, J=18.42 Hz, 2H), 2.38 (br. s., 1H), 2.33 (br. s., 1H), 1.98 (d, J=7.45 Hz, 2H), 1.63 (d, J=11.84 Hz, 2H), 1.56 (br. s., 1H), 1.16-1.26 (m, 2H).

### Example 7

### Synthesis of 6-chloro-N-((1-(3-(4-chloro-3-fluorophenoxy)-2-hydroxypropyl)piperidin-4-yl)methyl)quinoline-2-carboxamide

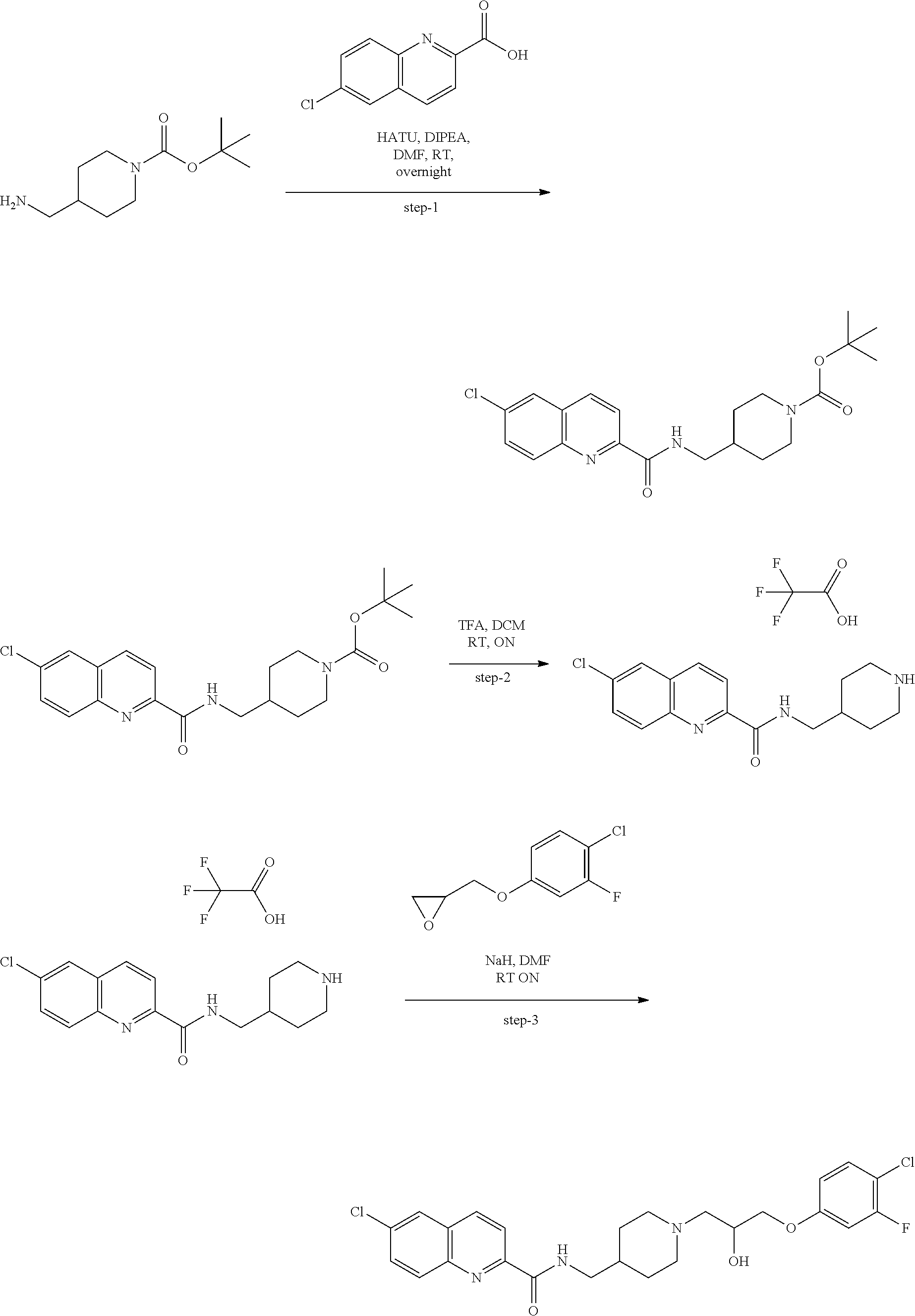

### Step 1—Synthesis of tert-butyl 4-((6-chloroquinoline-2-carboxamido)methyl)piperidine-1-carboxylate

To a solution of tert-butyl 4-(aminomethyl)piperidine-1-carboxylate (0.500 g, 2.33 mmol, 1.0 equiv) in DMF (10 mL) was added 6-chloroquinoline-2-carboxylic acid (0.433 g, 2.33 mmol, 1.0 equiv) and HATU (1.800 g, 4.66 mmol, 2.0 equiv) at RT. The reaction mixture was stirred for 10 minutes and then DIPEA (1.2 mL, 7.00 mmol, 3.0 equiv) was added. The resultant reaction mixture was allowed to stir at RT for overnight. Product formation was confirmed by 1H NMR. After the completion of reaction the reaction mixture was diluted with water (200 mL). The resulting solid was filtered off, washed with water (100 mL×2) and dried under vacuum to obtain tert-butyl 4-((6-chloroquinoline-2-carboxamido)methyl)piperidine-1-carboxylate (0.400 g, 42% Yield) as an off-white solid. LCMS 404.1 [M+H]⁺; ¹H NMR (400 MHz, DMSO-d₆) δ 8.99 (t, J=6.14 Hz, 1H), 8.54 (d, J=8.77 Hz, 1H), 8.11-8.31 (m, 3H), 7.88 (dd, J=8.77, 2.19 Hz, 1H), 3.93 (d, J=10.52 Hz, 2H), 3.26 (t, J=6.58 Hz, 2H), 2.62-2.76 (m, 3H), 1.81 (br. s., 1H), 1.66 (d, J=12.72 Hz, 2H), 1.38 (s, 9H), 0.90-1.15 (m, 2H).

### Step 2—Synthesis of 6-chloro-N-(piperidin-4-ylmethyl)quinoline-2-carboxamide 2,2,2-trifluoroacetate

To a stirred solution tert-butyl 4-((5-chlorobenzofuran-2-carboxamido)methyl)piperidine-1-carboxylate (0.400 g, 0.997 mmol, 1.0 equiv) in DCM (20 mL) was added TFA (0.4 mL) at RT. The reaction mixture was allowed to stir at RT overnight. Product formation was confirmed by 1H NMR. After the completion of reaction the DCM and excess of TFA was removed under reduced pressure. The crude product was crystallized in diethyl ether to 6-chloro-N-(piperidin-4-ylmethyl)quinoline-2-carboxamide 2,2,2-trifluoroacetate (0.400 g, 96% Yield) as an off-white solid. LCMS 304.0 [M+H]⁺; ¹H NMR (400 MHz, DMSO-d₆) δ 9.09 (t, J=6.14 Hz, 1H), 8.55 (d, J=8.77 Hz, 2H), 8.26 (d, J=2.19 Hz, 1H), 8.06-8.23 (m, 2H), 7.89 (dd, J=8.99, 2.41 Hz, 1H), 3.19-3.42 (m, 3H), 2.73-2.91 (m, 2H), 1.92 (br. s., 1H), 1.82 (d, J=14.47 Hz, 2H), 1.28-1.47 (m, 2H).

### Step 3—Synthesis of 6-chloro-N-((1-(3-(4-chloro-3-fluorophenoxy)-2-hydroxypropyl)piperidin-4-yl)methyl)quinoline-2-carboxamide

To a stirred solution of 6-chloro-N-(piperidin-4-ylmethyl)quinoline-2-carboxamide 2,2,2-trifluoroacetate (0.250 g, 0.599 mmol, 1.0 equiv) in DMF (10 mL) was added NaH (0.072 g, 1.798 mmol, 3.0 equiv) followed by the addition of 2-((4-chloro-3-fluorophenoxy)methyl)oxirane (0.145 g, 0.719 mmol, 1.2 equiv) at RT. The resulting reaction mixture was stir at RT for overnight. Product formation was confirmed by TLC and LCMS. Reaction mixture was diluted with water (50 mL) and extracted with EtOAc (75 mL×2). Combined organic layer was washed with water (4×50 mL), dried over anhydrous sodium sulfate and concentrated under reduced pressure to obtain crude which was crystallized in diethyl ether to obtain 6-chloro-N-((1-(3-(4-chloro-3-fluorophenoxy)-2-hydroxypropyl)piperidin-4-yl)methyl)quinoline-2-carboxamide (Compound 7—0.080 g, 26% Yield) as an off-white solid. LCMS 506.1 [M+H]⁺; ¹H NMR (400 MHz, DMSO-d₆) δ 8.94 (d, J=5.70 Hz, 1H), 8.54 (d, J=8.77 Hz, 1H), 8.25 (s, 1H), 8.16 (dd, J=16.88, 8.55 Hz, 1H), 7.88 (d, J=11.40 Hz, 1H), 7.45 (t, J=8.99 Hz, 1H), 7.07 (d, J=9.65 Hz, 1H), 6.83 (d, J=10.09 Hz, 1H), 4.85 (d, J=4.39 Hz, 1H), 4.00 (d, J=7.45 Hz, 1H), 3.88 (d, J=8.77 Hz, 1H), 3.25 (br. s., 2H), 2.76-3.00 (m, 2H), 2.20-2.43 (m, 3H), 1.96 (d, J=10.96 Hz, 2H), 1.62 (br. s., 2H), 1.22 (d, J=10.52 Hz, 2H).

### Example 8

### Synthesis of trans-6-chloro-N-(4-((3-(4-chlorophenoxy)-2-hydroxypropyl)amino)cyclohexyl)-1H-benzo[d]imidazole-2-carboxamide

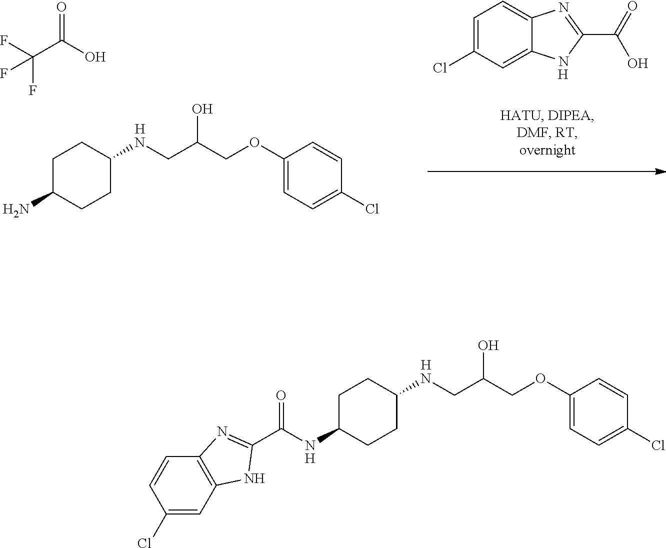

To a stirred solution of trans-1-((4-aminocyclohexyl)amino)-3-(4-chlorophenoxy)propan-2-ol 2,2,2-trifluoroacetate (0.1 g, 0.242 mmol, 1.0 equiv) and 2-chloro-1H-benzoimidazole-6-carboxylic acid (0.04 g, 0.242 mmol, 1.0 equiv) in DMF was added HATU (0.183 g, 0.484 mmol, 2.0 equiv) followed by the addition of DIPEA (0.062 g, 0.484 mmol, 2.0 equiv) at RT. The resulting reaction mixture was allowed to stir at RT for overnight. Product formation was confirmed by LCMS. The reaction mixture was quenched with water (50 mL) and extracted with ethyl acetate (50 mL×2). Combined organic layer was washed with water (25 mL×4), dried over anhydrous Na₂SO₄ and concentrated under reduced pressure. The crude product was purified by reversed phase HPLC to obtain trans-6-chloro-N-(4-((3-(4-chlorophenoxy)-2-hydroxypropyl)amino)cyclohexyl)-1H-benzo[d]imidazole-2-carboxamide (Compound 8—0.018 g, 16% Yield) as an off-white solid. LCMS 477.3 [M+H]⁺; ¹H NMR (400 MHz, DMSO-d₆) δ 1.19 (br. s., 2H), 1.52 (d, J=12.28 Hz, 2H), 1.80 (br. s., 2H), 1.95 (br. s., 2H), 2.78 (d, J=9.21 Hz, 2H), 3.77 (br. s., 2H), 3.89 (br. s., 1H), 3.95 (br. s., 1H), 6.91-7.04 (m, 1H), 7.32 (d, J=7.45 Hz, 3H), 7.53 (br. s., 1H), 7.72 (br. s., 2H), 8.22 (s, 2H), 8.80 (br. s., 2H).

### Example 9

### Synthesis of trans-2-(4-chloro-3-fluorophenoxy)-N-((4-(2-(4-chloro-3-fluorophenoxy)acetamido)cyclohexyl)methyl)acetamide

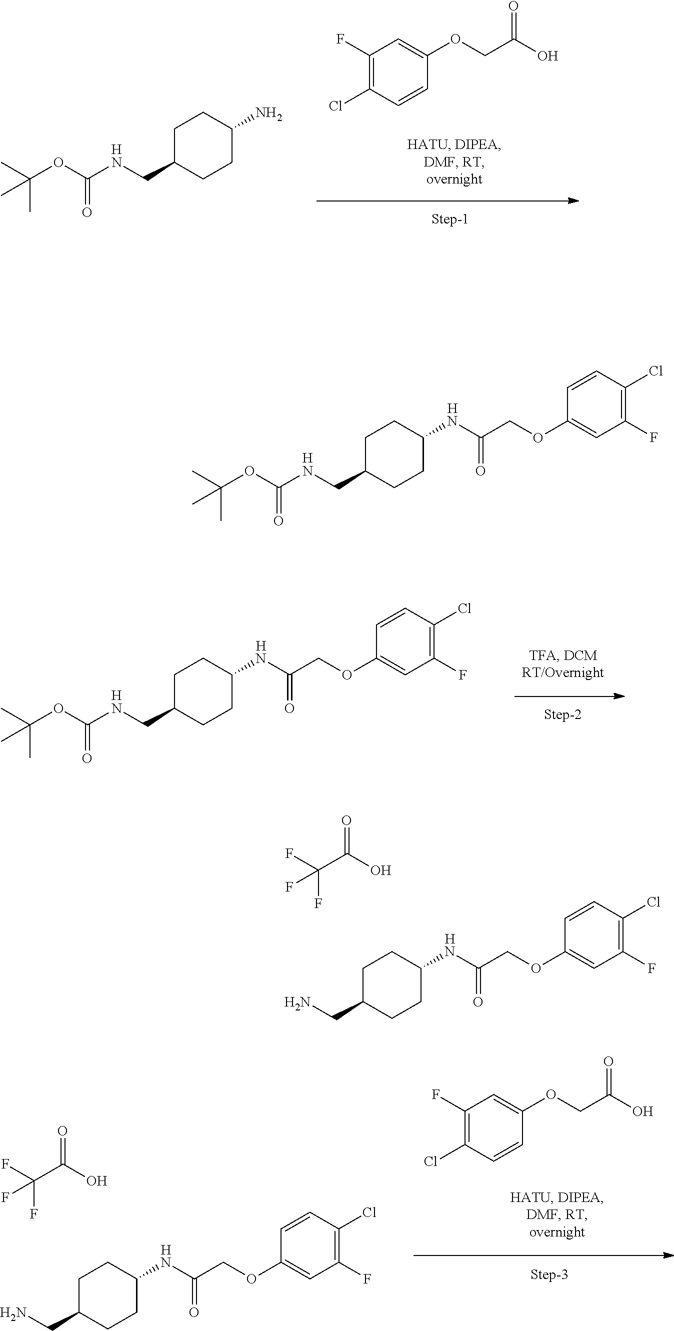

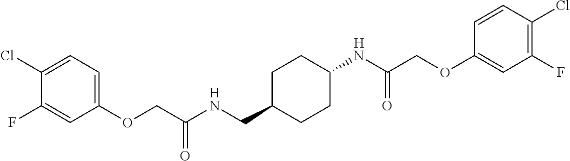

### Step 1—Synthesis of trans-tert-butyl ((4-(2-(4-chloro-3-fluorophenoxy)acetamido)cyclohexyl)methyl)carbamate

To a stirred solution of trans-tert-butyl ((4-aminocyclohexyl)methyl)carbamate (0.250 g, 1.09 mmol, 1.0 equiv) in DMF (05 mL) was added HATU (0.833 g, 2.1 mmol, 2.0 equiv) at RT and stirred for 10 minutes. 2-(4-chloro-3-fluorophenoxy)acetic acid (0.247 g, 1.20 mmol, 1.1 equiv) was added followed by the addition of DIPEA (0.56 mL, 3.2 mmol, 3.0 equiv). The resulting reaction mixture was allowed to stir at RT for overnight. Product formation was confirmed by ¹H NMR. The reaction mixture was diluted with water (50 mL) and extracted with EtOAc (100 mL×2). The combined organic layer was washed with water (50 mL), brine solution (50 mL×2), dried over anhydrous sodium sulfate and concentrated under reduced pressure to obtain trans-tert-butyl ((4-(2-(4-chloro-3-fluorophenoxy)acetamido)cyclohexyl)methyl)carbamate (0.190 g, 42% yield) as an off white solid. LCMS 415.1 [M+H]⁺; ¹H NMR (400 MHz, DMSO-d₆) δ 7.93 (d, J=8.33 Hz, 1H), 7.49 (t, J=8.99 Hz, 1H), 7.06 (dd, J=11.40, 2.63 Hz, 1H), 6.70-6.91 (m, 2H), 5.50 (d, J=7.89 Hz, 1H), 4.48 (s, 2H), 3.54 (d, J=7.89 Hz, 1H), 2.75 (q, J=5.99 Hz, 2H), 1.58-1.80 (m, 5H), 1.37 (s, 9H), 1.15-1.25 (m, 3H), 0.88-0.96 (m, 2H).

### Step 2—Synthesis of trans-N-(4-(aminomethyl)cyclohexyl)-2-(4-chloro-3-fluorophenoxy)acetamide 2,2,2-trifluoroacetate

To a stirred solution of trans-tert-butyl ((4-(2-(4-chloro-3-fluorophenoxy)acetamido)cyclohexyl)methyl)carbamate (0.190 g, 0.60 mmol, 1.0 equiv) in DCM (04 mL), was added TFA (0.5 mL) and the resultant reaction mixture was stirred at RT for 1 h under nitrogen atmosphere. Reaction was monitored by TLC and LCMS. After completion of reaction, the reaction mixture was concentrated under reduced pressure. The crude compound was washed with hexane (10 mL), crystallized in diethyl ether and dried under vacuum to obtain trans-N-(4-(aminomethyl)cyclohexyl)-2-(4-chloro-3-fluorophenoxy)acetamide 2,2,2-trifluoroacetate (0.200 g, Quant. yield) as a semi-solid. LCMS 315.0 [M+H]⁺; ¹H NMR (400 MHz, DMSO-d₆) δ 7.96 (d, J=8.33 Hz, 1H), 7.41-7.55 (m, 1H), 7.01-7.14 (m, 1H), 6.85 (dd, J=9.21, 1.75 Hz, 1H), 4.49 (s, 1H), 3.59 (dd, J=7.67, 3.73 Hz, 1H), 2.67 (t, J=5.92 Hz, 2H), 1.77 (d, J=4.82 Hz, 3H), 1.47 (d, J=6.14 Hz, 1H), 1.17-1.35 (m, 3H), 1.10-1.17 (m, 1H), 0.93-1.09 (m, 2H).

### Step 3—Synthesis of trans-2-(4-chloro-3-fluorophenoxy)-N-((4-(2-(4-chloro-3-fluorophenoxy)acetamido)cyclohexyl)methyl)acetamide

To a stirred solution of trans-N-(4-(aminomethyl)cyclohexyl)-2-(4-chloro-3-fluorophenoxy)acetamide 2,2,2-trifluoroacetate (0.200 g, 0.46 mmol, 1.0 equiv) in DMF (05 mL) was added HATU (0.355 g, 0.93 mmol, 2.0 equiv) at RT and stirred for 10 minutes. 2-(4-chloro-3-fluorophenoxy)acetic acid (0.095 g, 0.46 mmol, 1.0 equiv) was added followed by the addition of DIPEA (0.24 mL, 1.40 mmol, 3.0 equiv). The resulting reaction mixture was allowed to stir at RT for overnight. Product formation was confirmed by LCMS. The reaction mixture was diluted with water (50 mL). The resulting solid was filtered off, washed with water (20 mL×4) and dried under vacuum to gives crudeproduct which was purified by reverse phase of HPLC to obtain trans-2-(4-chloro-3-fluorophenoxy)-N-((4-(2-(4-chloro-3-fluorophenoxy)acetamido)cyclohexyl)methyl)acetamide (Compound 9—0.070 g, 30% Yield) a white solid. LCMS 501.3 [M+H]⁺; ¹H NMR (400 MHz, DMSO-d₆) δ 8.12 (t, J=5.92 Hz, 1H), 7.94 (d, J=7.89 Hz, 1H), 7.49 (t, J=8.55 Hz, 2H), 7.05 (d, J=2.63 Hz, 1H), 7.07 (d, J=2.63 Hz, 1H), 6.76-6.91 (m, 2H), 4.53 (s, 2H), 4.48 (s, 2H), 3.49-3.64 (m, 1H), 2.97 (t, J=6.14 Hz, 2H), 1.74 (d, J=10.09 Hz, 2H), 1.66 (d, J=11.84 Hz, 2H), 1.35 (d, J=13.15 Hz, 1H), 1.15-1.26 (m, 2H), 0.82-1.00 (m, 2H).

### Example 10

### Synthesis of trans-6-chloro-N-((4-(2-(4-chloro-3-fluorophenoxy)acetamido)cyclohexyl)methyl)quinoline-2-carboxamide

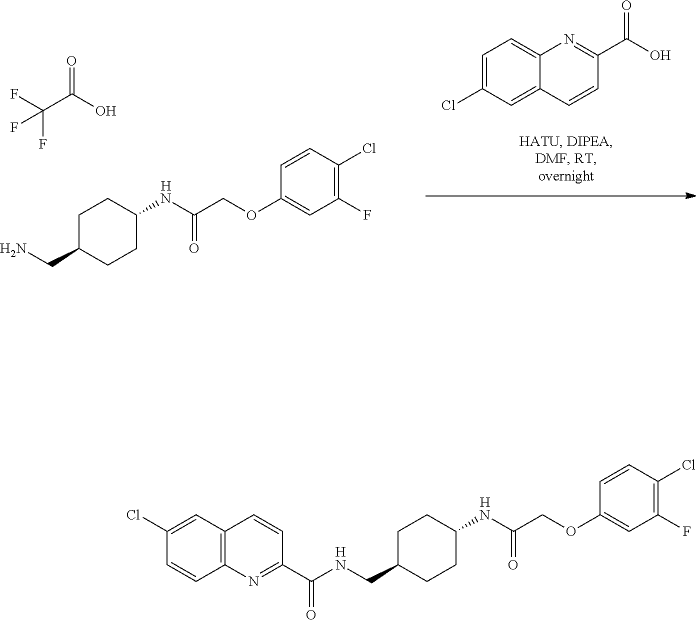

To a stirred solution of trans-N-(4-(aminomethyl)cyclohexyl)-2-(4-chloro-3-fluorophenoxy)acetamide 2,2,2-trifluoroacetate (0.200 g, 0.46 mmol, 1.0 equiv) in DMF (5 mL) was added HATU (0.355 g, 0.93 mmol, 2.0 equiv), 6-chloroquinoline-2-carboxylic acid (0.097 g, 0.46 mmol, 1.0 equiv) followed by the addition of DIPEA (0.3 mL, 1.40 mmol, 3.0 equiv). The resulting reaction mixture was allowed to stir at RT for overnight. Product formation was confirmed by LCMS. The reaction mixture was diluted with water (50 mL) and extracted with ethyl acetate (50 mL×3). Combined organic layer was washed with water (20 mL×4), dried over anhydrous sodium sulfate and concentrated. Crude product was crystallized in diethyl ether to obtain trans-6-chloro-N-((4-(2-(4-chloro-3-fluorophenoxy)acetamido)cyclohexyl)methyl)quinoline-2-carboxamide (Compound 10—0.100 g, 42% Yield) as an off-white solid. LCMS 504.3 [M+H]⁺; ¹H NMR (400 MHz, DMSO-d₆) δ 8.95 (t, J=6.36 Hz, 1H), 8.54 (d, J=8.33 Hz, 1H), 8.09-8.32 (m, 2H), 7.80-8.00 (m, 2H), 7.48 (t, J=8.77 Hz, 1H), 7.06 (dd, J=11.40, 2.63 Hz, 1H), 6.75-6.91 (m, 1H), 4.48 (s, 2H), 3.49-3.66 (m, 1H), 3.23 (t, J=6.58 Hz, 2H), 1.78 (d, J=10.52 Hz, 3H), 1.59 (br. s., 1H), 1.15-1.36 (m, 2H), 1.05 (q, J=11.69 Hz, 2H).

### Example 11

### Synthesis of trans-5-chloro-N-((4-(2-(4-chloro-3-fluorophenoxy)acetamido)cyclohexyl)methyl)benzofuran-2-carboxamide

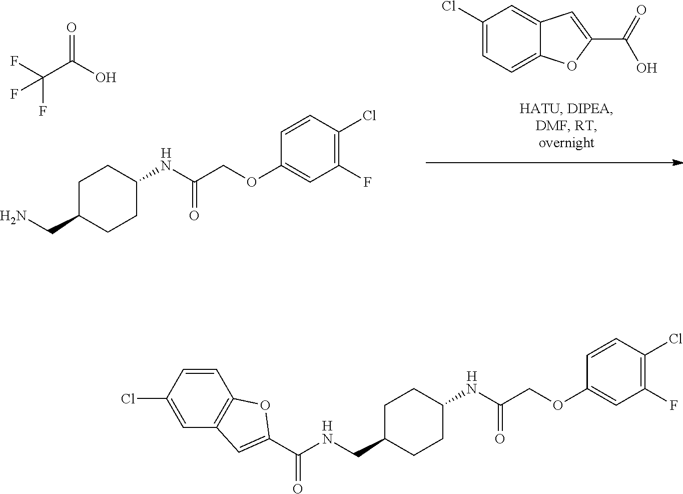

To a stirred solution of trans-N-(4-(aminomethyl)cyclohexyl)-2-(4-chloro-3-fluorophenoxy)acetamide 2,2,2-trifluoroacetate (0.200 g, 0.46 mmol, 1.0 equiv) in DMF (5 mL) was added HATU (0.355 g, 0.93 mmol, 2.0 equiv), 6-chloroquinoline-2-carboxylic acid (0.097 g, 0.46 mmol, 1.0 equiv) followed by the addition of DIPEA (0.3 mL, 1.40 mmol, 3.0 equiv). The resulting reaction mixture was allowed to stir at RT for overnight. Product formation was confirmed by LCMS. The reaction mixture was diluted with water (50 mL) and extracted with ethyl acetate (50 mL×3). Combined organic layer was washed with water (20 mL×4), dried over anhydrous sodium sulfate and concentrated. Crude product was crystallized in diethyl ether to obtain trans-5-chloro-N-((4-(2-(4-chloro-3-fluorophenoxy)acetamido)cyclohexyl)methyl)benzofuran-2-carboxamide (Compound 11—0.050 g, 21% Yield) as an off-white solid. LCMS 493.3 [M+H]⁺; ¹H NMR (400 MHz, DMSO-d₆) δ 8.80 (t, J=5.70 Hz, 1H), 7.95 (d, J=8.33 Hz, 1H), 7.87 (d, J=2.19 Hz, 1H), 7.70 (d, J=8.77 Hz, 1H), 7.38-7.55 (m, 2H), 7.06 (dd, J=11.40, 2.63 Hz, 1H), 6.84 (dd, J=8.77, 1.75 Hz, 1H), 4.48 (s, 2H), 3.58 (d, J=8.33 Hz, 1H), 3.12 (t, J=6.36 Hz, 2H), 1.77 (br. s., 4H), 1.52 (br. s., 1H), 1.15-1.26 (m, 2H), 1.01 (d, J=13.15 Hz, 2H).

### Example 12

### Synthesis of trans-6-chloro-N-(4-((2-(4-chloro-3-fluorophenoxy)acetamido)methyl)cyclohexyl)quinoline-2-carboxamide

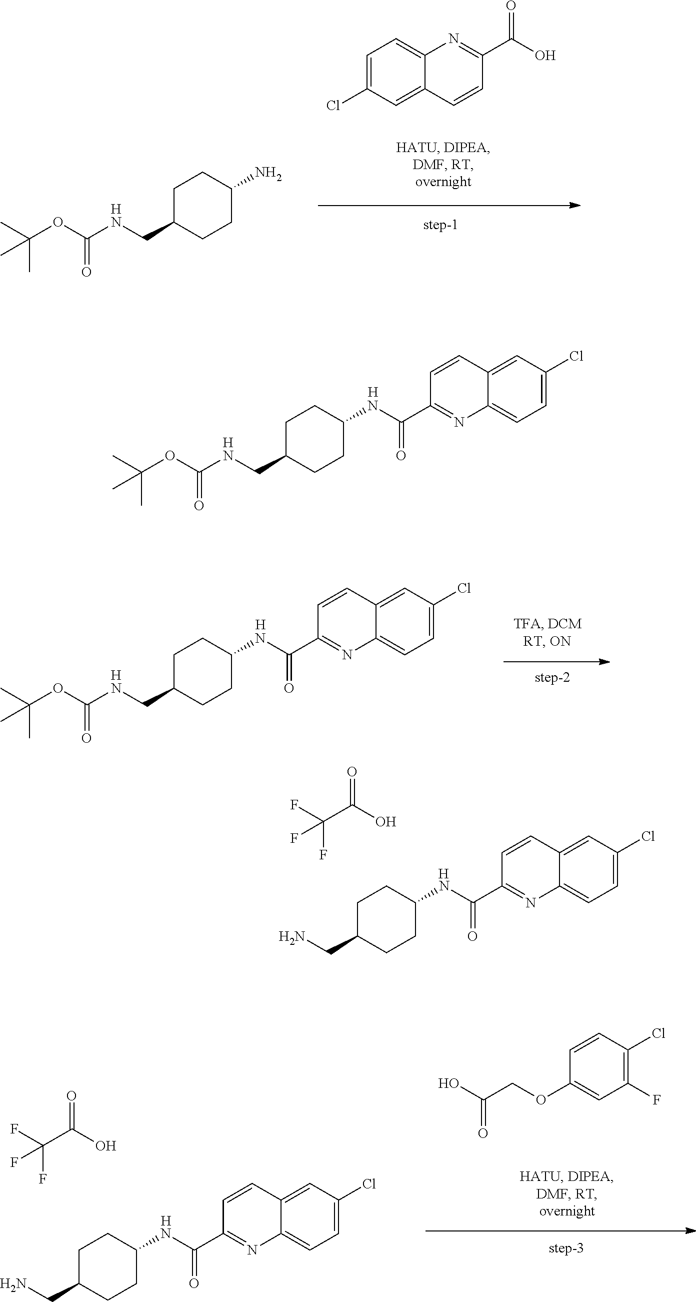

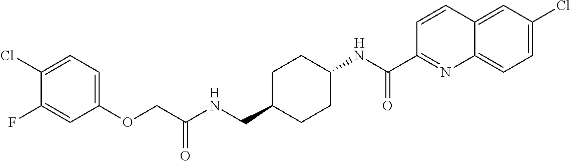

### Step 1—Synthesis of trans-tert-butyl ((4-(6-chloroquinoline-2-carboxamido)cyclohexyl)methyl)carbamate

To a stirred solution of trans-tert-butyl ((4-aminocyclohexyl)methyl)carbamate (0.180 g, 0.789 mmol, 1.0 equiv) in DMF (5 mL) was added HATU (0.600 g, 1.57 mmol, 2.0 equiv) at RT and stirred for 10 minutes. 6-chloroquinoline-2-carboxylic acid (0.163 g, 0.789 mmol, 1.1 equiv) was added followed by the addition of DIPEA (0.5 mL, 2.36 mmol, 3.0 equiv). The resulting reaction mixture was allowed to stir at RT for overnight. Product formation was confirmed by TLC and LCMS. The reaction mixture was diluted with water (100 mL). The resulting solid was filtered off and dried under vacuum. The crude product was enriched by flash chromatography (5% MeOH in DCM as an eluent) to obtain trans-tert-butyl ((4-(6-chloroquinoline-2-carboxamido)cyclohexyl)methyl)carbamate (0.100 g, 57% yield) as an off white solid. LCMS 418.1 [M+H]⁺; ¹H NMR (400 MHz, DMSO-d₆) δ 8.61 (d, J=8.77 Hz, 1H), 8.54 (d, J=8.33 Hz, 1H), 8.08-8.25 (m, 3H), 7.88 (dd, J=8.99, 2.41 Hz, 1H), 3.80 (br. s., 1H), 2.80 (t, J=6.36 Hz, 2H), 1.88 (d, J=10.09 Hz, 2H), 1.74 (d, J=12.28 Hz, 2H), 1.41-1.50 (m, 2H), 1.32-1.41 (m, 9H), 1.20-1.32 (m, 2H), 0.91-1.06 (m, 2H).

### Step 2—Synthesis of trans-N-(4-(aminomethyl)cyclohexyl)-6-chloroquinoline-2-carboxamide 2,2,2-trifluoroacetate

To a stirred solution of trans-tert-butyl ((4-(6-chloroquinoline-2-carboxamido)cyclohexyl)methyl)carbamate (0.100 g, 0.239 mmol, 1.0 equiv) in DCM (5 mL), was added TFA (0.2 mL) and the resultant reaction mixture was stirred at RT for 1 h under nitrogen atmosphere. Reaction was monitored by TLC and LCMS. After completion of reaction, the reaction mixture was concentrated under reduced pressure to obtain trans-N-(4-(aminomethyl)cyclohexyl)-6-chloroquinoline-2-carboxamide 2,2,2-trifluoroacetate (0.100 g, 97% Yield) as a yellow semi-solid. LCMS 318.1 [M+H]⁺; ¹H NMR (400 MHz, DMSO-d₆) δ 8.68 (d, J=8.77 Hz, 1H), 8.55 (d, J=8.77 Hz, 1H), 8.26 (d, J=2.19 Hz, 1H), 8.16 (d, J=9.21 Hz, 1H), 8.19 (d, J=8.77 Hz, 1H), 7.83-7.92 (m, 1H), 7.70 (br. s., 2H), 3.83 (d, J=8.33 Hz, 1H), 3.61 (d, J=6.58 Hz, 1H), 3.04-3.16 (m, 1H), 2.59-2.77 (m, 3H), 1.82-1.98 (m, 2H), 1.45-1.61 (m, 2H), 0.99-1.18 (m, 2H).

### Step 3—Synthesis of trans-6-chloro-N-(4-((2-(4-chloro-3-fluorophenoxy)acetamido)methyl)cyclohexyl)quinoline-2-carboxamide

To a stirred solution of trans-N-(4-(aminomethyl)cyclohexyl)-6-chloroquinoline-2-carboxamide 2,2,2-trifluoroacetate (0.100 g, 0.232 mmol, 1.0 equiv) in DMF (05 mL) was added HATU (0.176 g, 0.464 mmol, 2.0 equiv) at RT and stirred for 10 minutes. 2-(4-chloro-3-fluorophenoxy)acetic acid (0.047 g, 0.232 mmol, 1.0 equiv) was added followed by the addition of DIPEA (0.2 mL). The resulting reaction mixture was allowed to stir at RT for overnight. Product formation was confirmed by LCMS. The reaction mixture was diluted with water (50 mL). The resulting solid was filtered off, washed with water (20 mL×4) and dried under vacuum to gives crude product which was purified by flash chromatography (0-5% MeOH in DCM as an eluent) to obtain trans-6-chloro-N-(4-((2-(4-chloro-3-fluorophenoxy)acetamido)methyl)cyclohexyl)quinoline-2-carboxamide (Compound 12—0.060 g, 52% Yield) as an off-white solid. LCMS 504.3 [M+H]⁺; ¹H NMR (400 MHz, DMSO-d₆) δ 8.60 (d, J=8.77 Hz, 1H), 8.52 (d, J=8.33 Hz, 1H), 8.09-8.25 (m, 3H), 7.81-7.87 (m, 1H), 7.49 (t, J=8.77 Hz, 1H), 7.06 (dd, J=11.40, 2.63 Hz, 1H), 6.77-6.89 (m, 1H), 4.53 (s, 2H), 3.71-3.88 (m, 1H), 2.99 (t, J=6.58 Hz, 2H), 1.85 (d, J=9.21 Hz, 2H), 1.71 (d, J=12.28 Hz, 2H), 1.36-1.52 (m, 3H), 0.92-1.07 (m, 2H).

### Example 13

### Synthesis of trans-5-chloro-N-(4-((2-(4-chloro-3-fluorophenoxy)acetamido)methyl)cyclohexyl)benzofuran-2-carboxamide

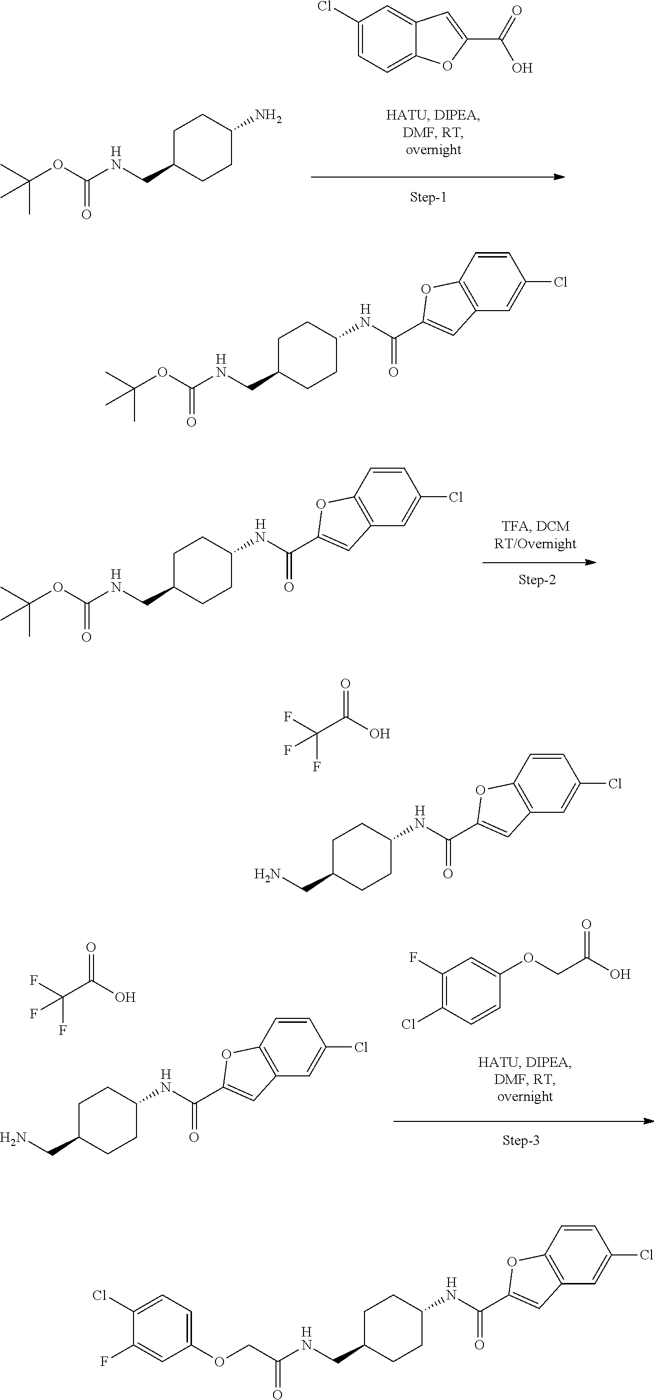

### Step 1—Synthesis of trans-tert-butyl ((4-(5-chlorobenzofuran-2-carboxamido)cyclohexyl)methyl)carbamate

To a stirred solution of trans-tert-butyl ((4-aminocyclohexyl)methyl)carbamate (0.140 g, 0.614 ininol, 1.0 equiv) in DMF (5 mL) was added HATU (0.466 g, 1.228 ininol, 2.0 equiv) at RT and stirred for 10 minutes. 5-chlorobenzofuran-2-carboxylic acid (0.120 g, 0.614 mmol, 1.0 equiv) was added followed by the addition of DIPEA (0.2 mL, 1.176 mmol, 3.0 equiv). The resulting reaction mixture was allowed to stir at RT for overnight. Product formation was confirmed by ¹H NMR. The reaction mixture was diluted with water (100 ml). The resulting solid was filtered off and washed with water, dried under vacuum to obtain trans-tert-butyl ((4-(5-chlorobenzofuran-2-carboxamido)cyclohexyl)methyl)carbamate (0.14 g, 48% yield) as an off white solid. LCMS 407.1 [M+H]⁺; ¹H NMR (400 MHz, DMSO-d₆) δ 8.56 (d, J=8.33 Hz, 1H), 7.86 (d, J=2.19 Hz, 1H), 7.69 (d, J=9.21 Hz, 1H), 7.39-7.57 (m, 2H), 6.84 (t, J=5.92 Hz, 1H), 3.71 (br. s., 1H), 2.71-2.84 (m, 2H), 1.83 (d, J=10.96 Hz, 2H), 1.64-1.76 (m, 2H), 1.32-1.42 (m, 9H), 1.13-1.28 (m, 2H), 0.96 (d, J=12.28 Hz, 3H).

### Step 2—Synthesis of trans-N-(4-(aminomethyl)cyclohexyl)-5-chlorobenzofuran-2-carboxamide 2,2,2-trifluoroacetate

To a stirred solution of tert-butyl (((1r,4r)-4-(5-chlorobenzofuran-2-carboxamido)cyclohexyl)methyl)carbamate (0.120 g, 0.294 mmol, 1.0 equiv) in DCM (4 mL), was added TFA (0.2 mL) and the resultant reaction mixture was stirred at RT for 1 h under nitrogen atmosphere. Reaction was monitored by TLC and LCMS. After completion of reaction, the reaction mixture was concentrated under reduced pressure to obtain crude compound which was washed with hexane (10 mL) and diethyl ether (10 ml), dried under vacuum to obtain trans-N-(4-(aminomethyl)cyclohexyl)-5-chlorobenzofuran-2-carboxamide 2,2,2-trifluoroacetate (0.120 g, 82% yield) as a yellow semi-solid. LCMS 307.0 [M+H]⁺; ¹H NMR (400 MHz, DMSO-d₆) δ 8.61 (d, J=7.89 Hz, 1H), 7.86 (s, 1H), 7.62-7.80 (m, 3H), 7.38-7.58 (m, 2H), 3.76 (d, J=7.89 Hz, 1H), 2.62-2.74 (m, 2H), 1.88 (br. s., 1H), 1.69-1.85 (m, 3H), 1.52 (br. s., 1H), 1.28-1.45 (m, 2H), 0.97-1.16 (m, 2H).

### Step 3—Synthesis of trans-5-chloro-N-(4-((2-(4-chloro-3-fluorophenoxy)acetamido)methyl)cyclohexyl)benzofuran-2-carboxamide

To a stirred solution of trans-N-(4-(aminomethyl)cyclohexyl)-5-chlorobenzofuran-2-carboxamide 2,2,2-trifluoroacetate (0.120 g, 0.392 mmol, 1.0 equiv) in DMF (05 mL) was added HATU (0.297 g, 0.7842 mmol, 2.0 equiv) at RT and stirred for 10 minutes. Then 2-(4-chloro-3-fluorophenoxy)acetic acid (0.079 g, 0.392 mmol, 1.0 equiv) was added followed by the addition of DIPEA (0.24 mL, 1.176 mmol, 3.0 equiv). The resulting reaction mixture was allowed to stir at RT for overnight. Product formation was confirmed by LCMS. The reaction mixture was diluted with water (50 mL). The resulting solid was filtered off, washed with water (20 mL×4) and dried under vacuum to gives crude product which was purified by reverse phase of HPLC to obtain trans-5-chloro-N-(4-((2-(4-chloro-3-fluorophenoxy)acetamido)methyl)cyclohexyl)benzofuran-2-carboxamide (Compound 13—0.020 g, 10% Yield) as a white solid. LCMS 493.1 [M+H]⁺; ¹H NMR (400 MHz, DMSO-d₆) δ 8.57 (d, J=7.89 Hz, 1H), 8.14 (br. s., 1H), 7.86 (br. s., 1H), 7.69 (d, J=7.89 Hz, 1H), 7.48 (s, 1H), 7.51 (s, 1H), 7.07 (d, J=10.96 Hz, 1H), 6.86 (d, J=7.02 Hz, 1H), 4.54 (br. s., 2H), 3.74 (br. s., 1H), 2.99 (br. s., 2H), 1.80 (br. s., 2H), 1.70 (d, J=11.84 Hz, 2H), 1.34 (d, J=10.96 Hz, 3H), 0.98 (d, J=12.28 Hz, 2H).

### Example 14

### Synthesis of trans-2-(4-chloro-3-fluorophenoxy)-N-((4-((3-(4-chloro-3-fluorophenoxy)-2-hydroxypropyl)amino)cyclohexyl)methyl)acetamide

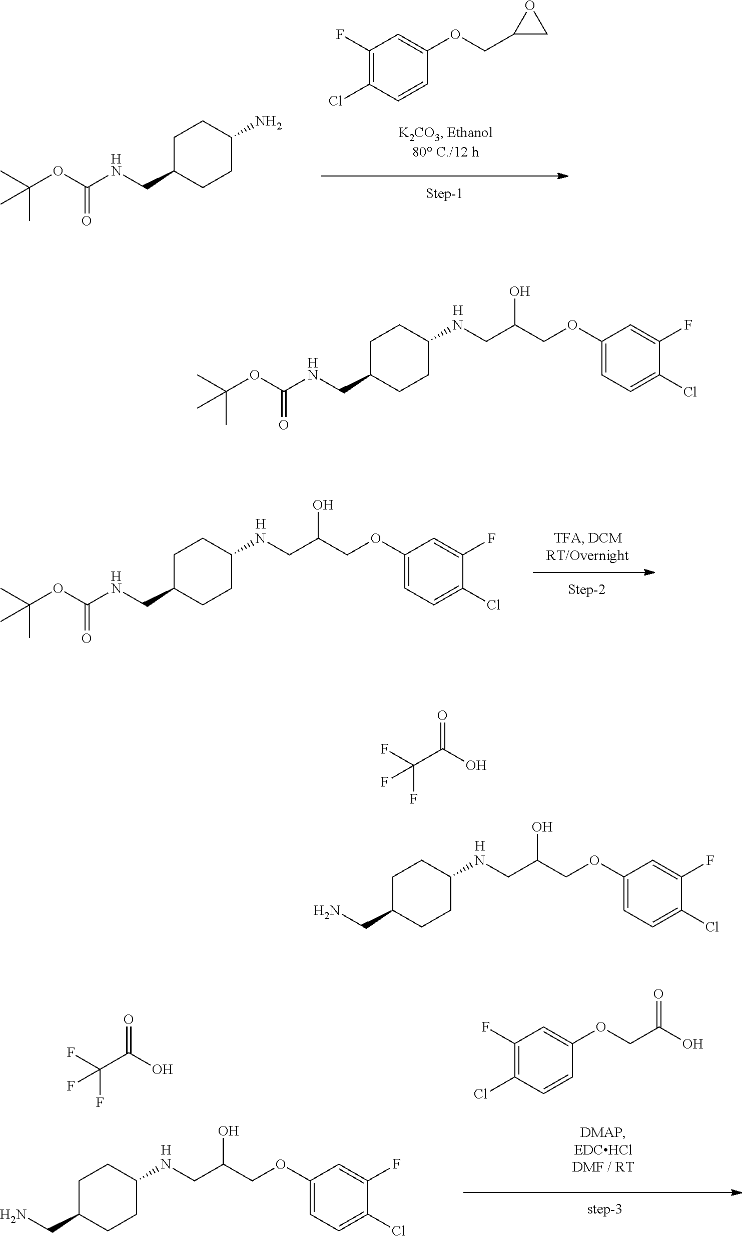

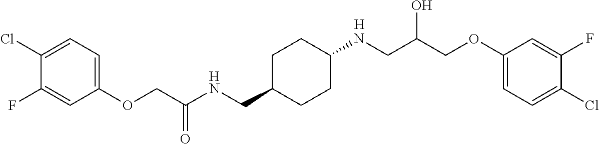

### Step 1—Synthesis of trans-tert-butyl ((4-((3-(4-chloro-3-fluorophenoxy)-2-hydroxypropyl)amino)cyclohexyl)methyl)carbamate

To a stirred solution of trans-tert-butyl ((4-aminocyclohexyl)methyl)carbamate (0.200 g, 0.614 mmol, 1.0 equiv) in Ethanol (5 mL) was added K2CO3 (0.363 g, 2.631 mmol, 3.0 equiv) at RT and stirred for 10 minutes. 2-((4-chloro-3-fluorophenoxy)methyl) oxirane (0.265 g, 1.315 mmol, 1.5 equiv) was added. The resulting reaction mixture was allowed to stir at 80 for overnight. Product formation was confirmed by ¹H NMR. After completion of reaction the solvent was evaporated under reduced pressure and the resulting residue was diluted with water (50 ml), extracted with DCM (100 ml). The organic layer was washed with water (50 mL), brine solution (50 mL×2), dried over anhydrous sodium sulfate and concentrated under reduced pressure to obtain trans-tert-butyl ((4-((3-(4-chloro-3-fluorophenoxy)-2-hydroxypropyl)amino)cyclohexyl)methyl)carbamate (0.150 g, 39% yield) as an off white solid. LCMS 431.5 [M+H]⁺; ¹H NMR (400 MHz, DMSO-d₆) δ 7.46 (t, J=8.77 Hz, 1H), 7.07 (dd, J=11.40, 2.63 Hz, 1H), 6.70-6.88 (m, 2H), 5.76 (s, 1H), 3.99 (d, J=5.26 Hz, 1H), 3.83-3.95 (m, 2H), 3.33 (br. s., 3H), 2.75 (t, J=6.14 Hz, 2H), 2.67 (br. s., 1H), 1.89 (br. s., 2H), 1.67 (d, J=12.28 Hz, 2H), 1.28-1.51 (m, 9H), 1.23 (br. s., 2H), 1.01 (d, J=10.96 Hz, 2H), 0.72-0.90 (m, 2H).

### Step 2—Synthesis of trans-1-((4-(aminomethyl)cyclohexyl)amino)-3-(4-chloro-3-fluorophenoxy)propan-2-ol 2,2,2-trifluoroacetate

To a stirred solution of trans-tert-butyl ((4-((3-(4-chloro-3-fluorophenoxy)-2-hydroxypropyl)amino)cyclohexyl)methyl)carbamate (0.150 g, 0.348 mmol, 1.0 equiv) in DCM (05 mL), was added TFA (0.4 mL) and the resultant reaction mixture was stirred at RT for 4 h under nitrogen atmosphere. Reaction was monitored by TLC and LCMS. After completion of reaction, the reaction mixture was concentrated under reduced pressure to obtain trans-1-((4-(aminomethyl)cyclohexyl)amino)-3-(4-chloro-3-fluorophenoxy)propan-2-ol 2,2,2-trifluoroacetate (0.150 g, 97% Yield) as a yellow semi-solid. LCMS 331.3 [M+H]⁺; ¹H NMR (400 MHz, DMSO-d₆) δ 8.61 (br. s., 1H), 8.50 (br. s., 1H), 7.80 (br. s., 2H), 7.50 (t, J=8.99 Hz, 1H), 7.09 (dd, J=11.40, 2.63 Hz, 1H), 6.87 (d, J=1.75 Hz, 1H), 5.96 (br. s., 1H), 4.14 (br. s., 1H), 4.01 (d, J=5.26 Hz, 2H), 3.16 (br. s., 1H), 3.02 (br. s., 2H), 2.63-2.72 (m, 2H), 1.96-2.15 (m, 2H), 1.84 (d, J=12.72 Hz, 2H), 1.25-1.41 (m, 2H), 0.90-1.06 (m, 2H).

### Step 3—Synthesis of trans-2-(4-chloro-3-fluorophenoxy)-N-((4-((3-(4-chloro-3-fluorophenoxy)-2-hydroxypropyl)amino)cyclohexyl)methyl)acetamide

To a stirred solution of trans-1-((4-(aminomethyl)cyclohexyl)amino)-3-(4-chloro-3-fluorophenoxy)propan-2-ol 2,2,2-trifluoroacetate (0.70 g, 0.212 mmol, 1.0 equiv) in DMF (04 mL) was added DMAP (0.64 g, 0.530 mmol, 2.0 equiv), EDC.HCL (0.81 mg, 0.424 mmol, 2.0 equiv) and stirred for 10 minutes at RT. 2-(4-chloro-3-fluorophenoxy)acetic acid (0.079 g, 0.392 mmol, 1.0 equiv) was added followed by the addition of DIPEA (0.24 mL, 1.176 mmol, 3.0 equiv). The resulting reaction mixture was allowed to stir at RT for overnight. Product formation was confirmed by LCMS. The reaction mixture was diluted with water (50 mL) and extracted with EtOAc (100 mL×2). The combined organic layer was washed with water (50 mL×4), brine solution (50 mL×2), dried over anhydrous sodium sulfate and concentrated under reduced pressure. The crude product was purified by flash chromatography (0-5% MeOH in DCM) to obtain trans-2-(4-chloro-3-fluorophenoxy)-N-((4-((3-(4-chloro-3-fluorophenoxy)-2-hydroxypropyl)amino)cyclohexyl)methyl)acetamide (Compound 14—0.020 g, 31% Yield) as a white solid. LCMS 517.4 [M+H]⁺; ¹H NMR (400 MHz, DMSO-d₆) δ 8.14 (br. s., 1H), 7.50 (br. s., 2H), 7.08 (t, J=8.77 Hz, 2H), 6.85 (d, J=7.89 Hz, 2H), 4.54 (br. s., 2H), 4.07 (br. s., 1H), 3.99 (br. s., 2H), 2.97 (br. s., 3H), 2.67 (br. s., 1H), 2.33 (br. s., 2H), 2.00 (br. s., 1H), 1.91 (br. s., 1H), 1.68 (br. s., 2H), 1.37 (br. s., 1H), 1.23 (br. s., 2H), 0.90 (d, J=13.15 Hz, 3H).

### Example 15

### Synthesis of trans-5-chloro-N-((4-(2-(4-chlorophenoxy)acetamido)cyclohexyl)methyl)benzofuran-2-carboxamide

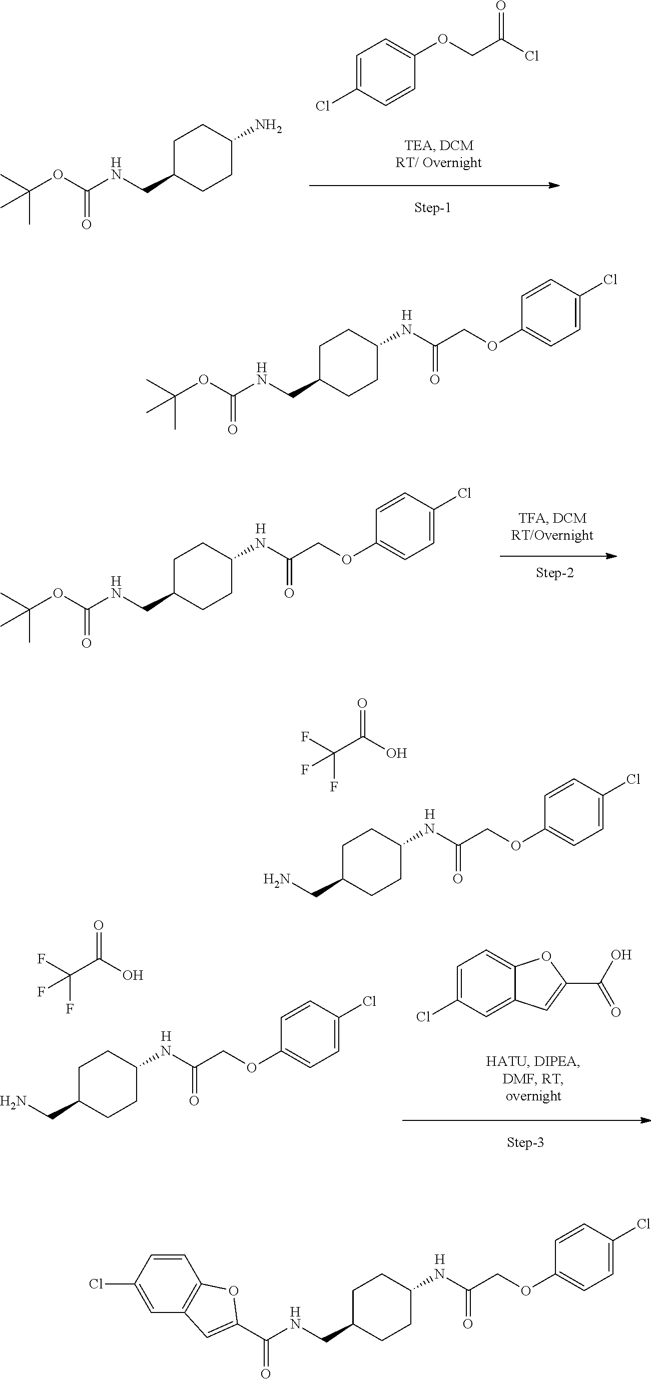

### Step 1—Synthesis of trans-tert-butyl ((4-(2-(4-chlorophenoxy)acetamido)cyclohexyl)methyl)carbamate

To a stirred solution of trans-tert-butyl ((4-aminocyclohexyl)methyl)carbamate (0.100 g, 0.438 mmol, 1.0 equiv) in DCM (04 mL) was added 2-(4-chlorophenoxy)acetyl chloride (0.107 g, 0.525 mmol, 1.2 equiv) followed by the addition of TEA (0.18 mL, 1.3 mmol, 3.0 equiv). The resulting reaction mixture was allowed to stir at RT for overnight. Product formation was confirmed by ¹H NMR. After completion of reaction the solvent was removed under reduced pressure and water was added to it. The resulting solid was filtered off, washed with water (10 mL×4) and dried under vacuum to obtain trans-tert-butyl ((4-(2-(4-chlorophenoxy)acetamido)cyclohexyl)methyl)carbamate (0.100 g, 57% yield) as an off white solid. LCMS 397.1 [M+H]⁺; ¹H NMR (400 MHz, DMSO-d₆) δ 7.88 (d, J=7.89 Hz, 1H), 7.34 (m, J=8.77 Hz, 2H), 6.96 (m, J=9.21 Hz, 2H), 6.80 (br. s., 1H), 4.43 (s, 2H), 3.54 (d, J=7.89 Hz, 1H), 2.76 (t, J=6.14 Hz, 2H), 1.61-1.84 (m, 4H), 1.37 (s, 9H), 1.06-1.28 (m, 3H), 0.85-0.98 (m, 2H).

### Step 2—Synthesis of trans-N-(4-(aminomethyl)cyclohexyl)-2-(4-chlorophenoxy)acetamide 2,2,2-trifluoroacetate

To a stirred solution of trans-tert-butyl ((4-(2-(4-chlorophenoxy)acetamido)cyclohexyl)methyl)carbamate (0.100 g, 0.252 mmol, 1.0 equiv) in DCM (04 mL), was added TFA (0.2 mL) and the resultant reaction mixture was stirred at RT for 2 h under nitrogen atmosphere. Reaction was monitored by TLC and LCMS. After completion of reaction, the reaction mixture was concentrated under reduced pressure. The crude compound which was washed with hexane (10 mL) and crystallized in diethyl ether to obtain trans-N-(4-(aminomethyl)cyclohexyl)-2-(4-chlorophenoxy)acetamide 2,2,2-trifluoroacetate (0.100 g, 97% Yield) as an off-white solid. LCMS 297.2 [M+H]⁺; ¹H NMR (400 MHz, DMSO-d₆) δ 7.92 (d, J=7.89 Hz, 1H), 7.67 (br. s., 2H), 7.24-7.39 (m, 2H), 6.88-7.05 (m, 2H), 4.44 (s, 2H), 3.58 (d, J=11.40 Hz, 1H), 2.57-2.73 (m, 2H), 1.90 (br. s., 1H), 1.76 (d, J=4.38 Hz, 3H), 1.48 (br. s., 1H), 1.17-1.30 (m, 2H), 0.95-1.09 (m, 2H).

### Step 3—Synthesis of trans-5-chloro-N-((4-(2-(4-chlorophenoxy)acetamido)cyclohexyl)methyl)benzofuran-2-carboxamide

To a stirred solution of trans-N-(4-(aminomethyl)cyclohexyl)-2-(4-chlorophenoxy)acetamide 2,2,2-trifluoroacetate (0.100 g, 0.24 mmol, 1.0 equiv) in DMF (04 mL) was added HATU (0.185 g, 0.48 mmol, 2.0 equiv) at RT and stirred for 10 minutes. 5-chlorobenzofuran-2-carboxylic acid (0.047 g, 0.24 mmol, 1.0 equiv) was added followed by the addition of DIPEA (0.13 mL, 0.73 mmol, 3.0 equiv). The resulting reaction mixture was allowed to stir at RT for overnight. Product formation was confirmed by LCMS. The reaction mixture was diluted with water (50 mL), resulting solid was filtered off, washed with water (20 mL×4) and dried under vacuum. The crude product was purified by flash chromatography (0-5% MeOH in DCM as an eluent) to obtain trans-5-chloro-N-((4-(2-(4-chlorophenoxy)acetamido)cyclohexyl)methyl)benzofuran-2-carboxamide (Compound 15—0.030 g, 26% Yield) as a white solid. LCMS 475.3[M+H]⁺; ¹H NMR (400 MHz, DMSO-d₆) δ 8.79 (t, J=5.70 Hz, 1H), 7.80-7.96 (m, 2H), 7.69 (d, J=8.33 Hz, 1H), 7.43-7.55 (m, 2H), 7.33 (m, J=8.77 Hz, 2H), 6.96 (m, J=9.21 Hz, 2H), 4.44 (s, 2H), 3.59 (br. s., 1H), 3.12 (t, J=6.36 Hz, 2H), 1.76 (d, J=9.21 Hz, 4H), 1.52 (br. s., 1H), 1.19-1.30 (m, 2H), 0.97-1.09 (m, 2H).

### Example 16

### Synthesis of trans-N-((4-(2-(4-chloro-3-fluorophenoxy)acetamido)cyclohexyl)methyl)-5-methoxybenzofuran-2-carboxamide

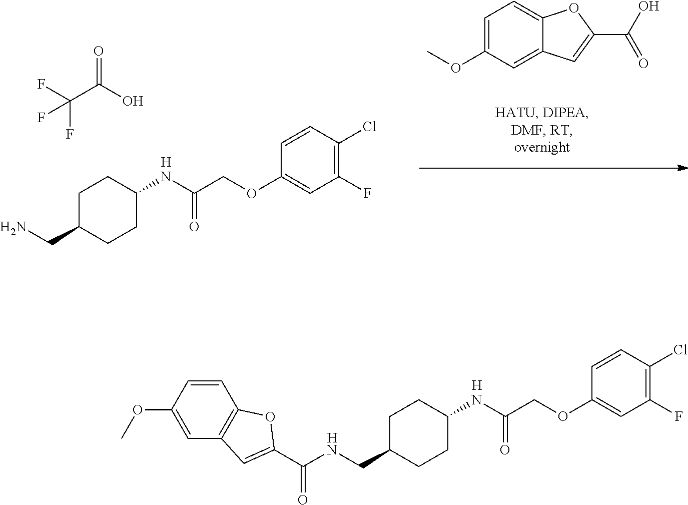

To a stirred solution of trans-N-(4-(aminomethyl)cyclohexyl)-2-(4-chloro-3-fluorophenoxy)acetamide 2,2,2-trifluoroacetate (0.100 g, 0.23 mmol, 1.0 equiv) in DMF (04 mL) was added HATU (0.177 g, 0.46 mmol, 2.0 equiv) at RT and stirred for 10 minutes. 5-methoxybenzofuran-2-carboxylic acid (0.044 g, 0.23 mmol, 1.0 equiv) was added followed by the addition of DIPEA (0.12 mL, 0.70 mmol, 3.0 equiv). The resulting reaction mixture was allowed to stir at RT for overnight. Product formation was confirmed by LCMS. The reaction mixture was diluted with water (50 mL) and extracted with EtOAc (100 mL×2). The combined organic layer was washed with water (50 mL), brine solution (50 mL×2), dried over anhydrous sodium sulfate and concentrated under reduced pressure. The crude product was purified by flash chromatography (0-5% MeOH in DCM as an eluent) to obtain trans-N-((4-(2-(4-chloro-3-fluorophenoxy)acetamido)cyclohexyl)methyl)-5-methoxybenzofuran-2-carboxamide (Compound 16-0.048 g, 42% Yield) as an off-white solid. LCMS 489.4 [M+H]⁺; ¹H NMR (400 MHz, DMSO-d₆) δ 8.66 (t, J=5.92 Hz, 1H), 7.93 (d, J=7.89 Hz, 1H), 7.37-7.62 (m, 3H), 7.25 (d, J=2.63 Hz, 1H), 6.99-7.12 (m, 2H), 6.77-6.88 (m, 1H), 4.48 (s, 2H), 3.80 (s, 2H), 3.51-3.62 (m, 1H), 3.12 (t, J=6.36 Hz, 2H), 2.69 (s, 1H), 1.77 (br. s., 3H), 1.52 (br. s., 1H), 1.13-1.30 (m, 3H), 0.94-1.09 (m, 2H).

### Example 17

### Synthesis of trans-4-chloro-N-((4-(2-(4-chloro-3-fluorophenoxy)acetamido)cyclohexyl)methyl)benzamide

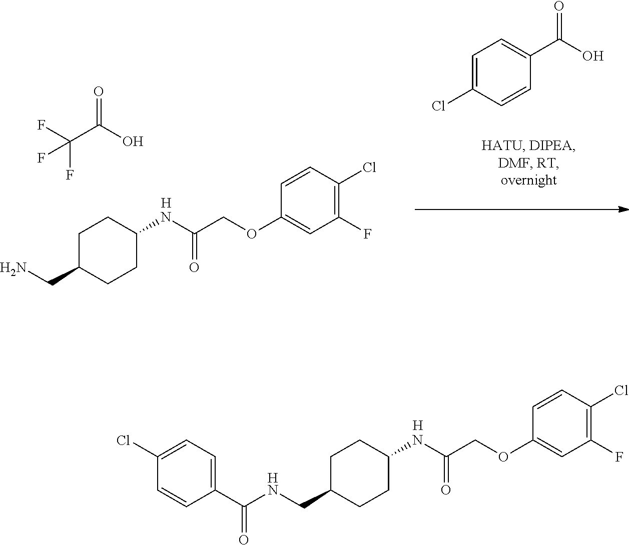

To a stirred solution of trans-N-(4-(aminomethyl)cyclohexyl)-2-(4-chloro-3-fluorophenoxy)acetamide 2,2,2-trifluoroacetate (0.100 g, 0.23 mmol, 1.0 equiv) in DMF (04 mL). 4-chlorobenzoic acid((0.36 g, 0.23 mmol, 1.0 equiv) was added HATU (0.177 g, 0.46 mmol, 2.0 equiv) at RT and stirred for 10 minutes. 2-(4-chloro-3-fluorophenoxy)acetic acid (0.095 g, 0.46 mmol, 1.0 equiv) was added followed by the addition of DIPEA (0.12 mL, 0.70 mmol, 3.0 equiv). The resulting reaction mixture was allowed to stir at RT for overnight. Product formation was confirmed by LCMS. The reaction mixture was diluted with water (50 mL). The resulting solid was filtered off, washed with water (20 mL×4), dried under vacuum to gives crude product which was purified by reverse phase of HPLC to obtain trans-4-chloro-N-((4-(2-(4-chloro-3-fluorophenoxy)acetamido)cyclohexyl)methyl)benzamide (Compound 17—0.036 g, 34% Yield) as an off-white solid. LCMS 453.3 [M+H]⁺; ¹H NMR (400 MHz, DMSO-d₆) δ 8.53 (t, J=5.48 Hz, 1H), 7.93 (d, J=7.89 Hz, 1H), 7.86 (d, J=8.33 Hz, 2H), 7.35-7.64 (m, 3H), 7.06 (dd, J=11.40, 2.63 Hz, 1H), 6.77-6.90 (m, 1H), 4.48 (s, 2H), 3.59 (dd, J=7.89, 3.95 Hz, 1H), 3.10 (t, J=6.14 Hz, 2H), 1.76 (d, J=9.21 Hz, 4H), 1.50 (br. s., 1H), 1.16-1.32 (m, 2H), 0.93-1.07 (m, 2H).

### Example 18

### Synthesis of trans-N-((4-(2-(4-chloro-3-fluorophenoxy)acetamido)cyclohexyl)methyl)-4-(trifluoromethoxy)benzamide

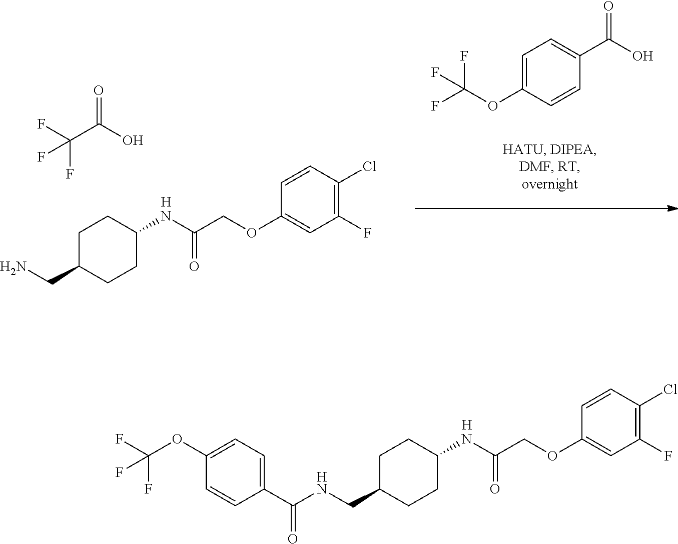

To a stirred solution of trans-N-(4-(aminomethyl)cyclohexyl)-2-(4-chloro-3-fluorophenoxy)acetamide 2,2,2-trifluoroacetate (0.100 g, 0.23 mmol, 1.0 equiv) in DMF (04 mL) was added HATU (0.177 g, 0.46 mmol, 2.0 equiv) at RT and stirred for 10 minutes. 4-(trifluoromethoxy)benzoic acid (0.048 g, 0.233 mmol, 1.0 equiv) was added followed by the addition of DIPEA (0.12 mL, 0.70 mmol, 3.0 equiv). The resulting reaction mixture was allowed to stir at RT for overnight. Product formation was confirmed by LCMS. The reaction mixture was diluted with water (50 ml). The resulting solid was filtered off, washed with water (10 mL×4) and dried under vacuum to gives crude product which was purified by flash chromatography (0-5% MeOH in DCM as an eluent) to trans-N-((4-(2-(4-chloro-3-fluorophenoxy)acetamido)cyclohexyl)methyl)-4-(trifluoromethoxy)benzamide (Compound 18—0.050 g, 42% Yield) as an off-white solid. LCMS 503.4 [M+H]⁺; ¹H NMR (400 MHz, DMSO-d₆) δ 8.52-8.62 (m, 1H), 7.82-8.09 (m, 2H), 7.35-7.56 (m, 2H), 7.06 (dd, J=11.40, 3.07 Hz, 1H), 6.84 (d, J=8.77 Hz, 1H), 4.48 (s, 2H), 3.58 (d, J=8.77 Hz, 1H), 3.11 (t, J=6.14 Hz, 2H), 1.77 (d, J=9.65 Hz, 4H), 1.50 (br. s., 1H), 1.15-1.32 (m, 2H), 0.87-1.12 (m, 2H).

### Example 19

### Synthesis of trans-5-chloro-N-((4-(2-(4-chloro-3-fluorophenoxy)acetamido)cyclohexyl)methyl)picolinamide

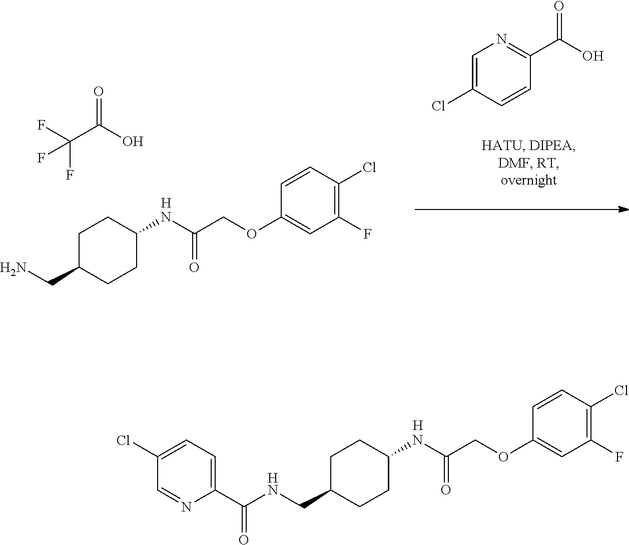

To a stirred solution of trans-N-(4-(aminomethyl)cyclohexyl)-2-(4-chloro-3-fluorophenoxy)acetamide 2,2,2-trifluoroacetate (0.100 g, 0.23 mmol, 1.0 equiv) in DMF (04 mL) was added HATU (0.177 g, 0.46 mmol, 2.0 equiv) at RT and stirred for 10 minutes. 5-chloropicolinic acid (0.036 g, 0.23 mmol, 1.0 equiv) was added followed by the addition of DIPEA (0.12 mL, 0.70 mmol, 3.0 equiv). The resulting reaction mixture was allowed to stir at RT for overnight. Product formation was confirmed by LCMS. The reaction mixture was diluted with water (50 mL). The resulting solid was filtered off, washed with water (20 mL×4) and dried under vacuum to gives crude product which was purified by flash chromatography (0-5% MeOH in DCM as an eluent) to obtain trans-5-chloro-N-((4-(2-(4-chloro-3-fluorophenoxy)acetamido)cyclohexyl)methyl)picolinamide (Compound 19—0.042 g, 40% Yield) as a white solid. LCMS 454.3 [M+H]⁺; ¹H NMR (400 MHz, DMSO-d₆) δ 8.78 (br. s., 1H), 8.67 (s, 1H), 8.10 (d, J=8.77 Hz, 1H), 8.01 (d, J=8.33 Hz, 1H), 7.91 (d, J=7.89 Hz, 1H), 7.47 (t, J=8.99 Hz, 1H), 7.03 (d, J=11.40 Hz, 1H), 6.82 (d, J=9.21 Hz, 1H), 4.46 (s, 2H), 3.54 (br. s., 1H), 3.13 (t, J=6.36 Hz, 2H), 1.72 (t, J=14.03 Hz, 4H), 1.52 (br. s., 1H), 1.11-1.27 (m, 2H), 0.91-1.04 (m, 2H).

### Example 20

### Synthesis of trans-N-((4-(2-(4-chloro-3-fluorophenoxy)acetamido)cyclohexyl)methyl)-5-(trifluoromethyl)picolinamide

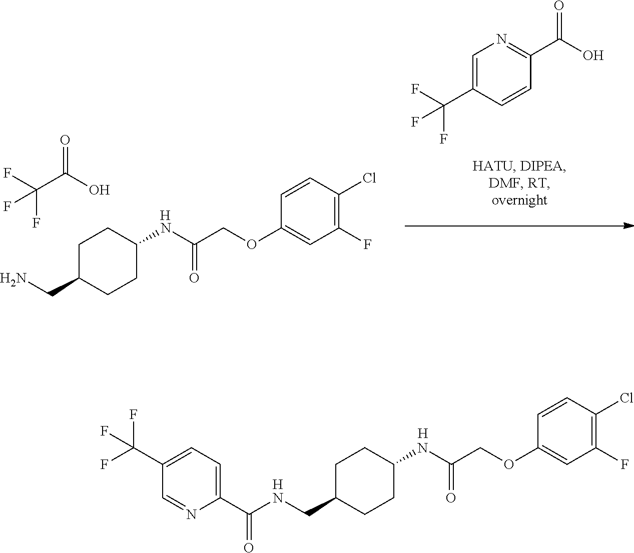

To a stirred solution of trans-N-(4-(aminomethyl)cyclohexyl)-2-(4-chloro-3-fluorophenoxy)acetamide 2,2,2-trifluoroacetate (0.100 g, 0.23 mmol, 1.0 equiv) in DMF (04 mL) was added HATU (0.177 g, 0.46 mmol, 2.0 equiv) at RT and stirred for 10 minutes. 5-(trifluoromethyl)picolinic acid (0.044 g, 0.233 mmol, 1.0 equiv) was added followed by the addition of DIPEA (0.12 mL, 0.70 mmol, 3.0 equiv). The resulting reaction mixture was allowed to stir at RT for overnight. Product formation was confirmed by LCMS. The reaction mixture was diluted with water (50 ml) The resulting solid was filtered off, washed with water (10 mL×4) and dried under vacuum to gives crude product which was purified by flash chromatography (0-5% MeOH in DCM as an eluent) to obtain trans-N-((4-(2-(4-chloro-3-fluorophenoxy)acetamido)cyclohexyl)methyl)-5-(trifluoromethyl)picolinamide (Compound 20—0.064 g, 56% Yield) as an off-white solid. LCMS 488.4 [M+H]⁺; ¹H NMR (400 MHz, DMSO-d₆) δ 9.02 (s, 1H), 8.95 (br. s., 1H), 8.40 (d, J=9.65 Hz, 1H), 8.19 (d, J=8.33 Hz, 1H), 7.91 (d, J=8.77 Hz, 1H), 7.47 (t, J=8.55 Hz, 1H), 7.04 (dd, J=11.18, 2.85 Hz, 1H), 6.82 (d, J=6.58 Hz, 2H), 4.46 (s, 2H), 3.15 (d, J=6.58 Hz, 2H), 1.73 (t, J=12.94 Hz, 4H), 1.55 (br. s., 1H), 1.21 (br. s., 2H), 0.99 (d, J=10.52 Hz, 2H).

### Example 21

### Synthesis of trans-N-((4-(2-(4-chloro-3-fluorophenoxy)acetamido)cyclohexyl)methyl)-5-(difluoromethyl)pyrazine-2-carboxamide

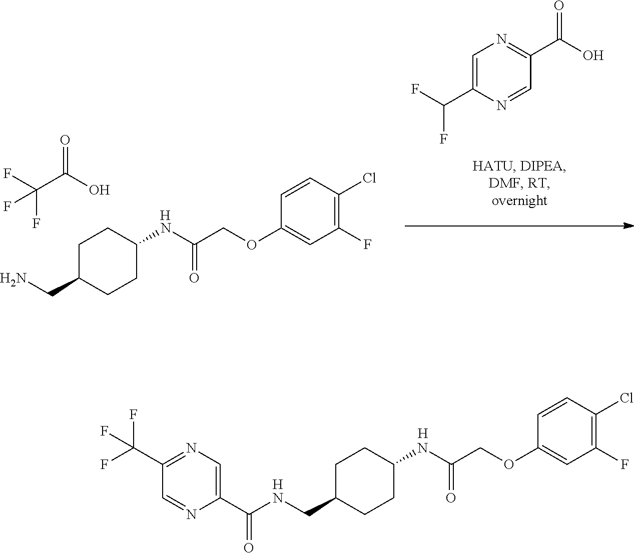

To a stirred solution of trans-N-(4-(aminomethyl)cyclohexyl)-2-(4-chloro-3-fluorophenoxy)acetamide 2,2,2-trifluoroacetate (0.100 g, 0.23 mmol, 1.0 equiv) in DMF (04 mL) was added HATU (0.177 g, 0.46 mmol, 2.0 equiv) at RT and stirred for 10 minutes. 5-(difluoromethyl)pyrazine-2-carboxylic acid (0.040 g, 0.233 mmol, 1.0 equiv) was added followed by the addition of DIPEA (0.12 mL, 0.70 mmol, 3.0 equiv). The resulting reaction mixture was allowed to stir at RT for overnight. Product formation was confirmed by LCMS. The reaction mixture was diluted with water (50 ml) The resulting solid was filtered off, washed with water (10 mL×4) and dried under vacuum to gives crude product which was purified by flash chromatography (0-5% MeOH in DCM as an eluent) to obtain trans-N-((4-(2-(4-chloro-3-fluorophenoxy)acetamido)cyclohexyl)methyl)-5-(difluoromethyl)pyrazine-2-carboxamide (Compound 21—0.040 g, 36% Yield) as an off-white solid. LCMS 471.4 [M+H]⁺; ¹H NMR (400 MHz, DMSO-d₆) δ 9.27 (s, 1H), 8.99-9.07 (m, 2H), 7.93 (d, J=8.33 Hz, 1H), 7.49 (t, J=8.77 Hz, 1H), 7.35 (s, 1H), 6.98-7.11 (m, 1H), 6.84 (dt, J=9.10, 1.37 Hz, 1H), 4.48 (s, 2H), 3.52-3.61 (m, 1H), 3.18 (t, J=6.58 Hz, 2H), 1.75 (t, J=10.74 Hz, 4H), 1.57 (br. s., 1H), 1.16-1.28 (m, 2H), 1.00-1.10 (m, 2H).

### Example 22

### Synthesis of trans-3-chloro-N-((4-(2-(4-chloro-3-fluorophenoxy)acetamido)cyclohexyl)methyl)benzamide

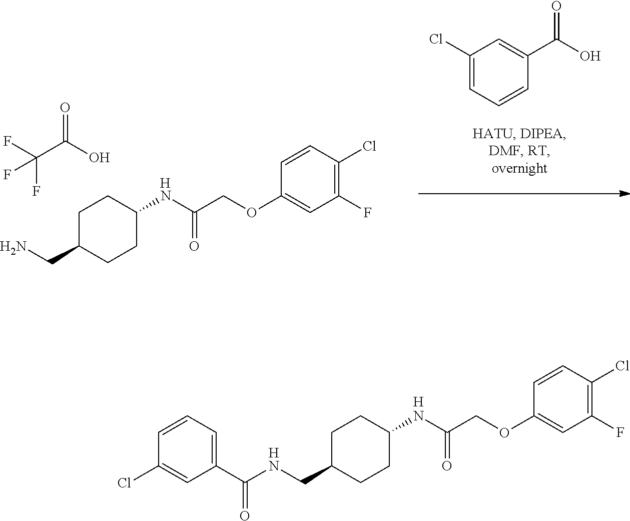

To a stirred solution of trans-N-(4-(aminomethyl)cyclohexyl)-2-(4-chloro-3-fluorophenoxy)acetamide 2,2,2-trifluoroacetate (0.100 g, 0.23 mmol, 1.0 equiv) in DMF (04 mL) was added HATU (0.177 g, 0.46 mmol, 2.0 equiv) at RT and stirred for 10 minutes. 3-chlorobenzoic acid (0.036 g, 0.233 mmol, 1.0 equiv) was added followed by the addition of DIPEA (0.12 mL, 0.70 mmol, 3.0 equiv). The resulting reaction mixture was allowed to stir at RT for overnight. Product formation was confirmed by LCMS. The reaction mixture was diluted with water (50 ml) The resulting solid was filtered off, washed with water (10 mL×4) and dried under vacuum to gives crude product which was purified by flash chromatography (0-5% MeOH in DCM as an eluent) to obtain trans-3-chloro-N-((4-(2-(4-chloro-3-fluorophenoxy)acetamido)cyclohexyl)methyl)benzamide (Compound 22—0.041 g, 39% Yield) as an off-white solid. LCMS 453.4 [M+H]⁺; ¹H NMR (400 MHz, DMSO-d₆) δ 8.57 (t, J=5.48 Hz, 1H), 7.95 (s, 1H), 7.88 (s, 1H), 7.80 (d, J=7.45 Hz, 1H), 7.59 (d, J=7.89 Hz, 1H), 7.40-7.55 (m, 2H), 7.06 (dd, J=11.62, 2.85 Hz, 1H), 6.84 (dd, J=8.77, 2.63 Hz, 1H), 4.48 (s, 2H), 3.58 (d, J=7.45 Hz, 1H), 3.11 (t, J=6.36 Hz, 2H), 1.77 (d, J=9.21 Hz, 4H), 1.50 (br. s., 1H), 1.17-1.26 (m, 2H), 0.94-1.06 (m, 2H).

### Example 23

### Synthesis of trans-4-chloro-N-((4-(2-(4-chloro-3-fluorophenoxy)acetamido)cyclohexyl)methyl)-3-fluorobenzamide

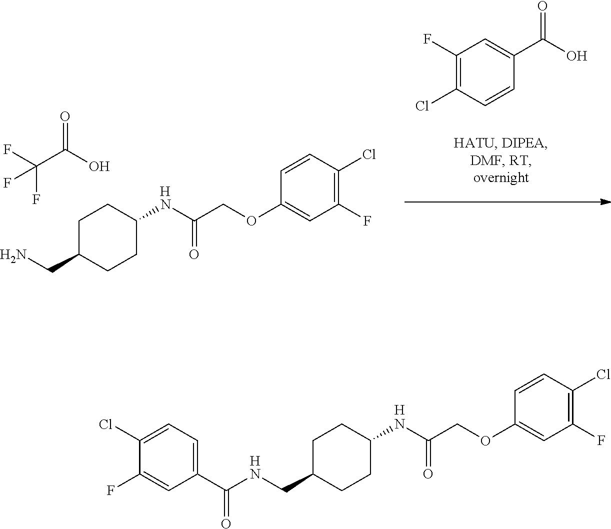

To a stirred solution of trans-N-(4-(aminomethyl)cyclohexyl)-2-(4-chloro-3-fluorophenoxy)acetamide 2,2,2-trifluoroacetate (0.100 g, 0.23 mmol, 1.0 equiv) in DMF (04 mL) was added HATU (0.177 g, 0.46 mmol, 2.0 equiv) at RT and stirred for 10 minutes. 4-chloro-3-fluorobenzoic acid (0.040 g, 0.233 mmol, 1.0 equiv) was added followed by the addition of DIPEA (0.12 mL, 0.69 mmol, 3.0 equiv). The resulting reaction mixture was allowed to stir at RT for overnight. Product formation was confirmed by LCMS. The reaction mixture was diluted with water (50 ml) The resulting solid was filtered off, washed with water (10 mL×4) and dried under vacuum to gives crude product which was purified by flash chromatography (0-5% MeOH in DCM as an eluent) to obtain trans-4-chloro-N-((4-(2-(4-chloro-3-fluorophenoxy)acetamido)cyclohexyl)methyl)-3-fluorobenzamide (Compound 23—0.075 g, 68% Yield) as a white solid. LCMS 471.3 [M+H]⁺; ¹H NMR (400 MHz, DMSO-d₆) δ 8.53-8.67 (m, 1H), 7.93 (d, J=8.33 Hz, 1H), 7.78-7.87 (m, 1H), 7.59-7.77 (m, 2H), 7.49 (t, J=8.99 Hz, 1H), 7.06 (dd, J=11.18, 2.85 Hz, 1H), 6.84 (dd, J=8.99, 1.53 Hz, 1H), 4.48 (s, 2H), 3.58 (d, J=8.33 Hz, 1H), 3.11 (t, J=6.36 Hz, 2H), 1.77 (d, J=9.65 Hz, 4H), 1.50 (br. s., 1H), 1.11-1.28 (m, 2H), 0.93-1.10 (m, 2H).

### Example 24

### Synthesis of trans-7-chloro-N-((4-(2-(4-chloro-3-fluorophenoxy)acetamido)cyclohexyl)methyl)quinoline-2-carboxamide

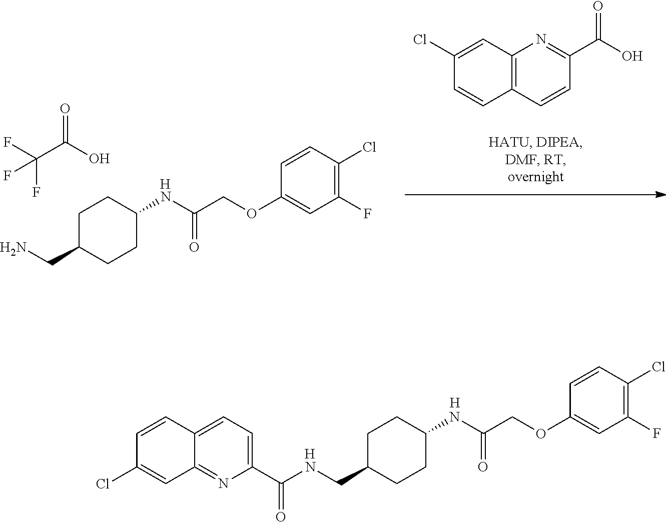

To a stirred solution of trans-N-(4-(aminomethyl)cyclohexyl)-2-(4-chloro-3-fluorophenoxy)acetamide 2,2,2-trifluoroacetate (0.100 g, 0.23 mmol, 1.0 equiv) in DMF (04 mL) was added HATU (0.177 g, 0.46 mmol, 2.0 equiv) at RT and stirred for 10 minutes. 7-chloroquinoline-2-carboxylic acid (0.048 g, 0.233 mmol, 1.0 equiv) was added followed by the addition of DIPEA (0.12 mL, 0.70 mmol, 3.0 equiv). The resulting reaction mixture was allowed to stir at RT for overnight. Product formation was confirmed by LCMS. The reaction mixture was diluted with water (50 mL) and extracted with EtOAc (100 mL×2). The combined organic layer was washed with water (50 mL), brine solution (50 mL×2), dried over anhydrous sodium sulfate and concentrated under reduced pressure. The crude product was enriched by flash chromatography (0-5% MeOH in DCM as an eluent) followed by the purification of reversed phase HPLC to obtain trans-7-chloro-N-((4-(2-(4-chloro-3-fluorophenoxy)acetamido)cyclohexyl)methyl)quinoline-2-carboxamide (Compound 24—0.015 g, 12% Yield) as an off-white solid. LCMS 504.4 [M+H]⁺; ¹H NMR (400 MHz, DMSO-d₆) δ 8.92 (t, J=6.36 Hz, 1H), 8.62 (d, J=8.33 Hz, 1H), 8.03-8.33 (m, 3H), 7.94 (d, J=8.33 Hz, 1H), 7.77 (dd, J=8.77, 2.19 Hz, 1H), 7.48 (t, J=8.99 Hz, 1H), 7.06 (dd, J=11.40, 3.07 Hz, 1H), 6.84 (dd, J=8.77, 1.75 Hz, 1H), 4.48 (s, 2H), 3.58 (br. s., 1H), 3.23 (t, J=6.80 Hz, 2H), 1.78 (d, J=11.40 Hz, 4H), 1.59 (br. s., 1H), 1.16-1.35 (m, 2H), 0.95-1.14 (m, 2H).

### Example 25

### Synthesis of trans-6-chloro-N-(4-((5-chlorobenzofuran-2-carboxamido)methyl)cyclohexyl)quinoline-2-carboxamide

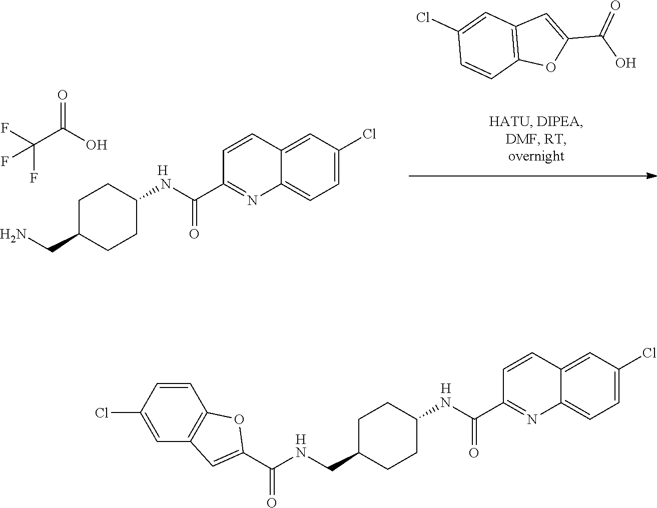

To a stirred solution of trans-2-(4-(aminomethyl)cyclohexyl)-1-(6-chloroquinolin-2-yl)ethan-1-one 2,2,2-trifluoroacetate (0.200 g, 0.46 mmol, 1.0 equiv) and 5-chlorobenzofuran-2-carboxylic acid, (0.090 g, 0.46 mmol, 1.0 equiv) in DMF (7 mL) was added HATU (0.349 g, 0.92 mmol, 2.0 equiv) followed by the addition of DIPEA(0.118 g, 0.92 mmol, 2.0 equiv). The resulting reaction mixture was allowed to stir for overnight at RT. Product formation was confirmed by LCMS. After the completion of reaction the reaction mixture was diluted with water (50 mL). The resulting solid was filtered off, washed with water (20 mL×4) and dried under vacuum. The crude product was purified by reverse phase HPLC to obtain trans-6-chloro-N-(4-((5-chlorobenzofuran-2-carboxamido)methyl)cyclohexyl)quinoline-2-carboxamide (Compound 25—0.075 g, 30% Yield) as an off-white solid. LCMS 496.4 [M+H]⁺; ¹H NMR (400 MHz, DMSO-d₆) δ 8.83 (t, J=5.70 Hz, 1H), 8.62 (d, J=8.77 Hz, 1H), 8.53 (d, J=8.33 Hz, 2H), 8.24 (d, J=2.19 Hz, 1H), 8.12-8.21 (m, 1H), 7.87-7.93 (m, 1H), 7.70 (d, J=9.21 Hz, 1H), 7.48 (dd, J=8.77, 2.19 Hz, 1H), 3.82 (d, J=8.33 Hz, 1H), 3.17 (t, J=6.36 Hz, 2H), 1.90 (d, J=10.52 Hz, 2H), 1.82 (d, J=11.84 Hz, 2H), 1.60 (br. s., 1H), 1.39-1.54 (m, 2H), 0.98-1.20 (m, 2H).

### Example 26

### Synthesis of trans-5-chloro-N-((4-(5-chlorobenzofuran-2-carboxamido)cyclohexyl)methyl)benzofuran-2-carboxamide

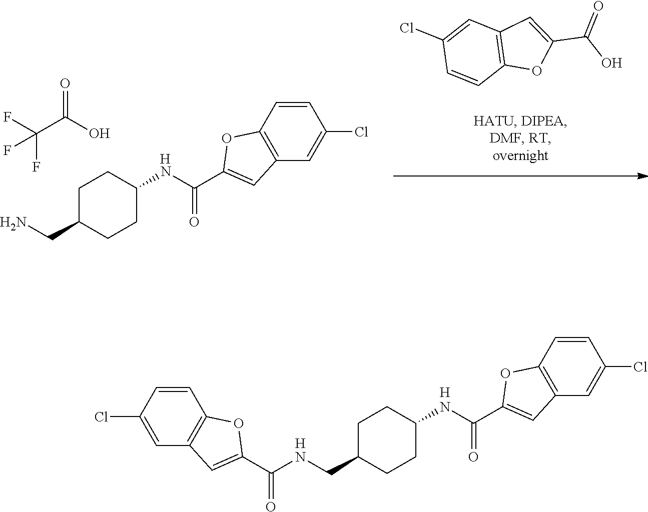

To a stirred solution of trans-N-(4-(aminomethyl)cyclohexyl)-5-chlorobenzofuran-2-carboxamide 2,2,2-trifluoroacetate (0.200 g, 0.47 mmol, 1.0 equiv) and 5-chlorobenzofuran-2-carboxylic acid, (0.093 g, 0.47 mmol, 1.0 equiv) in DMF (05 mL) was added HATU (0.357 g, 0.94 mmol, 2.0 equiv) followed by the addition of DIPEA((0.121 g, 0.94 mmol, 2.0 equiv). The resulting reaction mixture was allowed to stir for overnight at RT. Product formation was confirmed by LCMS. After the completion of reaction the reaction mixture was diluted with water (50 mL). The resulting solid was filtered off, washed with water (20 mL×4) and dried under vacuum. The crude product was purified by reverse phase HPLC to obtain trans-5-chloro-N-((4-(5-chlorobenzofuran-2-carboxamido)cyclohexyl)methyl)benzofuran-2-carboxamide (Compound 26—0.1 g, 04% Yield) as a white solid. LCMS 485.3 [M+H]⁺; ¹H NMR (400 MHz, DMSO-d₆) δ 8.80 (br. s., 1H), 8.56 (s, 1H), 7.73-7.95 (m, 2H), 7.66-7.73 (m, 1H), 7.44-7.61 (m, 2H), 3.15 (t, J=6.36 Hz, 1H), 1.85 (d, J=12.72 Hz, 2H), 1.78 (br. s., 4H), 1.38 (d, J=10.09 Hz, 2H), 1.23 (br. s., 3H), 1.06 (d, J=12.72 Hz, 2H).

### Example 27

### Synthesis of trans-5-chloro-N-((4-((2-(4-chloro-3-fluorophenoxy)ethyl)amino)cyclohexyl)methyl)benzofuran-2-carboxamide

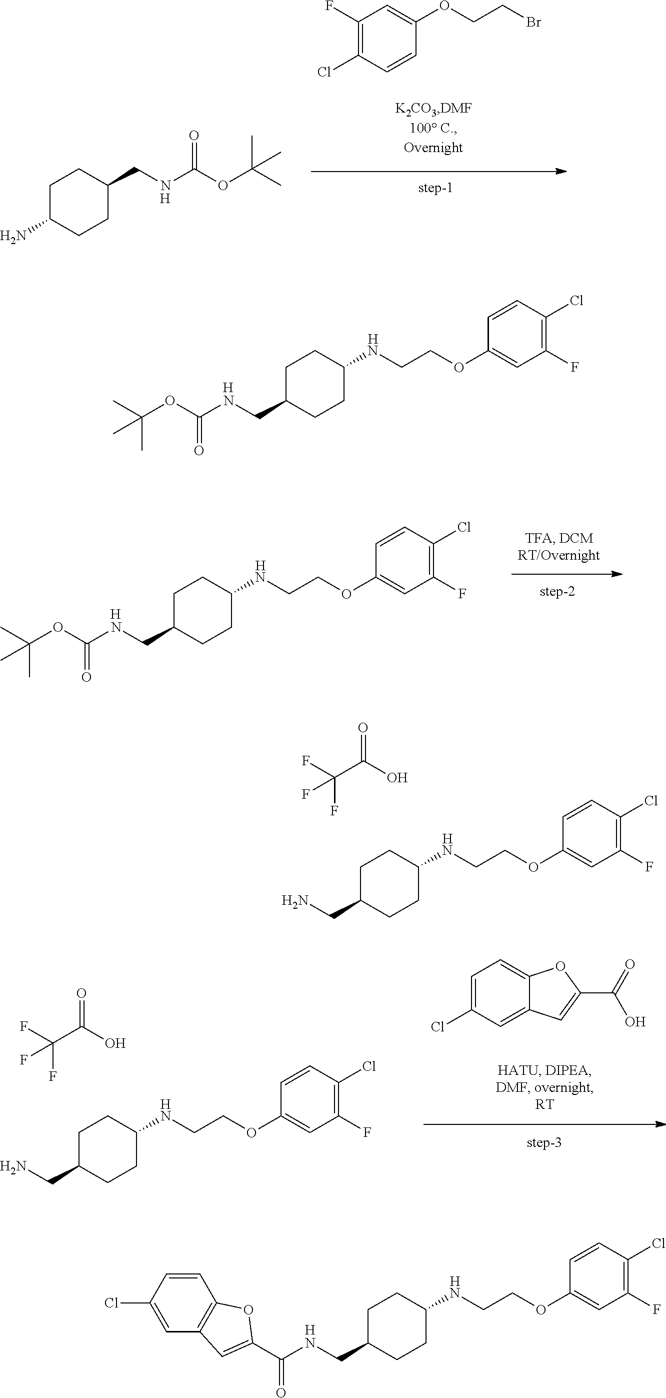

### Step 1—Synthesis of trans-tert-butyl ((4-((2-(4-chloro-3-fluorophenoxy)ethyl)amino)cyclohexyl)methyl)carbamate

To a stirred solution of trans-tert-butyl ((4-aminocyclohexyl)methyl)carbamate (0.100 g, 0.44 mmol, 1.0 equiv) in DMF (5 mL) was added K₂CO3 (0.057 g, 0.44 mmol, 1.0 equiv) followed by the addition of 4-(2-bromoethoxy)-1-chloro-2-fluorobenzene (0.122 g, 0.48 mmol, 1.1 equiv) at RT. The resulting reaction mixture was allowed to stir at 100° C. for overnight. Product formation was confirmed by LCMS. The reaction mixture was diluted with water (25 mL) and extracted with ethyl acetate (50 mL×2). Combined organic layer was washed with water (25 mL×4), dried over anhydrous Na2SO4 and concentrated under reduced pressure. The crude product was purified by flash chromatography (0-30% Ethyl acetate in Hexane as an eluent) to obtain trans-tert-butyl ((4-((2-(4-chloro-3-fluorophenoxy)ethyl)amino)cyclohexyl)methyl)carbamate (0.070 g, 40% Yield) as an off-white solid. LCMS 401.1 [M+H]⁺; ¹H NMR (400 MHz, DMSO-d₆) δ 7.45 (t, J=8.99 Hz, 1H), 7.06 (dd, J=11.62, 2.85 Hz, 1H), 6.70-6.89 (m, 2H), 4.00 (t, J=5.48 Hz, 2H), 2.88 (br. s., 2H), 2.75 (t, J=6.36 Hz, 2H), 1.85 (br. s., 2H), 1.66 (d, J=11.84 Hz, 2H), 1.37 (s, 9H), 1.23 (br. s., 2H), 0.76-1.04 (m, 4H).

### Step 2—Synthesis trans-4-(aminomethyl)-N-(2-(4-chloro-3-fluorophenoxy)ethyl)cyclohexan-1-amine 2,2,2-trifluoroacetate

To a stirred solution of trans-tert-butyl ((4-((2-(4-chloro-3-fluorophenoxy)ethyl)amino)cyclohexyl)methyl)carbamate of (0.07 g, 0.174 mmol, 1.0 equiv) in DCM (5 mL), was added TFA (0.5 mL) and the resultant reaction mixture was stirred at RT for overnight. Reaction was monitored by LCMS. After completion of reaction, the reaction mixture was concentrated under reduced pressure to obtain sticky crude compound which was triturated with diethyl ether, solid ppt. filtered off, dried under vacuum to obtain trans-4-(aminomethyl)-N-(2-(4-chloro-3-fluorophenoxy)ethyl)cyclohexan-1-amine 2,2,2-trifluoroacetate (0.070 g, Quant. Yield) as a white solid. LCMS 301.2 [M+H]⁺; ¹H NMR (400 MHz, DMSO-d₆) δ 8.63 (br. s., 1H), 7.69 (br. s., 2H), 7.54 (t, J=8.55 Hz, 1H), 7.13 (dd, J=11.40, 2.63 Hz, 1H), 6.89 (d, J=10.52 Hz, 1H), 4.26 (br. s., 1H), 3.08 (br. s., 1H), 2.67 (br. s., 2H), 2.10 (d, J=11.40 Hz, 2H), 1.84 (d, J=14.03 Hz, 2H), 1.49 (br. s., 2H), 1.19-1.38 (m, 2H), 1.10 (d, J=6.58 Hz, 1H), 1.00 (d, J=11.84 Hz, 2H).

### Step 3—Synthesis of trans-5-chloro-N-((4-((2-(4-chloro-3-fluorophenoxy)ethyl)amino)cyclohexyl)methyl)benzofuran-2-carboxamide

To a stirred solution of trans-4-(aminomethyl)-N-(2-(4-chloro-3-fluorophenoxy)ethyl)cyclohexan-1-amine 2,2,2-trifluoroacetate (0.1 g, 0.241 mmol, 1.0 equiv) and 5-chlorobenzofuran-2-carboxylic acid, (0.047 g, 0.241 mmol, 1.0 equiv) in DMF (5 mL) was added HATU (0.319 g, 0.82 mmol, 2.0 equiv) followed by the addition of DIPEA (0.108 g, 0.84 mmol, 2.0 equiv). The resulting reaction mixture was allowed to stir for overnight at RT. Product formation was confirmed by LCMS. After the completion of reaction the reaction mixture was diluted with water (50 mL). The resulting solid was filtered off, washed with water (20 mL×4) and dried under vacuum. The crude product was purified by reverse phase HPLC to obtain trans-5-chloro-N-((4-((2-(4-chloro-3-fluorophenoxy)ethyl)amino)cyclohexyl)methyl)benzofuran-2-carboxamide (Compound 27—0.01 g, 04% Yield) as a white solid. LCMS 479.3 [M+H]⁺; ¹H NMR (400 MHz, DMSO-d₆) δ 8.77 (t, J=5.92 Hz, 1H), 7.86 (d, J=2.19 Hz, 1H), 7.69 (d, J=8.77 Hz, 1H), 7.48-7.58 (m, 1H), 7.35-7.48 (m, 1H), 7.06 (dd, J=11.62, 2.85 Hz, 1H), 6.83 (dd, J=9.21, 1.75 Hz, 1H), 4.02 (t, J=5.70 Hz, 2H), 3.07-3.19 (m, 2H), 2.92 (t, J=5.48 Hz, 2H), 2.42 (br. s., 1H), 1.90 (br. s., 2H), 1.74 (d, J=9.21 Hz, 2H), 1.52 (br. s., 1H), 1.23 (s, 1H), 0.79-1.09 (m, 3H).

### Example 28

### Synthesis of trans-2-(4-chloro-3-fluorophenoxy)-N-(4-((3-(4-chlorophenyl)ureido)methyl)cyclohexyl)acetamide

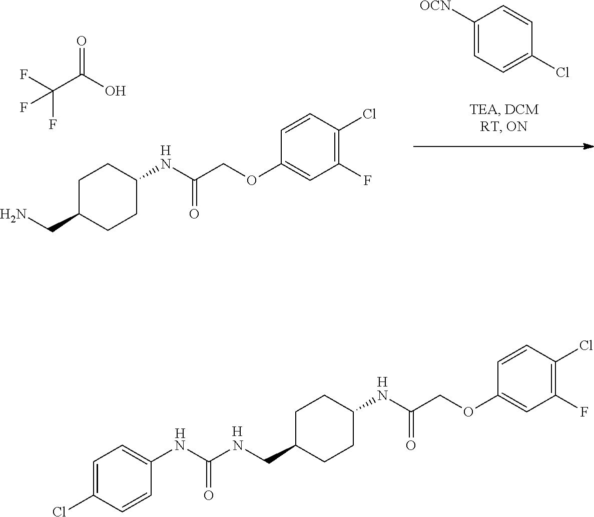

To a stirred suspended of trans-N-(4-(aminomethyl)cyclohexyl)-2-(4-chloro-3-fluorophenoxy)acetamide 2,2,2-trifluoroacetate (0.200 g, 0.467 mmol, 1.0 equiv) in DCM (20 mL) was added triethyl amine (0.142 g, 1.401 mmol, 3.0 equiv) followed by the addition of 1-chloro-4-isocyanatobenzene (0.086 g, 0.560 mmol, 1.2 equiv) at RT. The resulting reaction mixture was allowed to stir at RT for 2 h. After two hours stirring the resulting precipitate was filtered off, washed with excess DCM and dried under vacuum. The crude product was purified by flash chromatography (0-5% MeOH in DCM as an eluent) to obtain trans-2-(4-chloro-3-fluorophenoxy)-N-(4-((3-(4-chlorophenyl)ureido)methyl)cyclohexyl)acetamide (Compound 28—0.110 g, 50% Yield) as an off-white solid. LCMS 468.4 [M+H]⁺; ¹H NMR (400 MHz, DMSO-d₆) δ 8.50 (s, 1H), 7.93 (d, J=7.89 Hz, 1H), 7.49 (t, J=8.77 Hz, 1H), 7.40 (m, J=8.77 Hz, 2H), 7.24 (m, J=8.77 Hz, 2H), 7.06 (dd, J=11.40, 2.63 Hz, 1H), 6.84 (d, J=8.77 Hz, 1H), 6.22 (t, J=5.48 Hz, 1H), 4.48 (s, 2H), 3.49-3.65 (m, 1H), 2.94 (t, J=5.70 Hz, 2H), 1.65-1.87 (m, 3H), 1.35 (br. s., 1H), 1.23 (q, J=11.84 Hz, 2H), 0.84-1.11 (m, 2H).

### Example 29

### Synthesis of trans-2-(4-chloro-3-fluorophenoxy)-N-(4-((2-(4-chlorophenyl)acetamido)methyl)cyclohexyl)acetamide

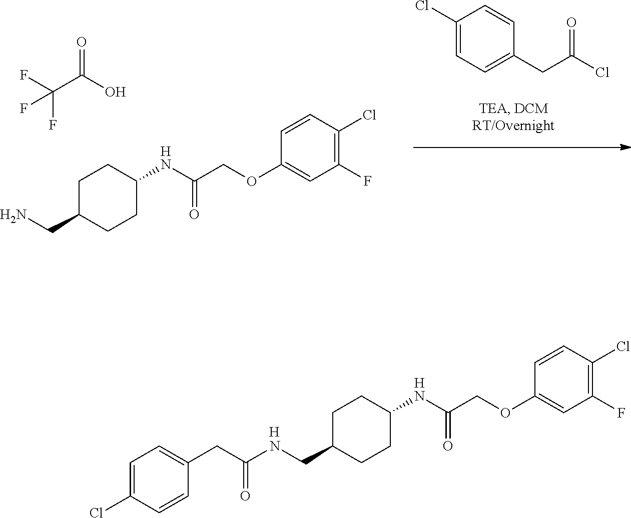

To a stirred solution of trans-N-(4-(aminomethyl)cyclohexyl)-2-(4-chloro-3-fluorophenoxy)acetamide 2,2,2-trifluoroacetate (0.100 g, 0.23 mmol, 1.0 equiv) in DCM (5 mL) was added TEA (0.09 mL, 0.699 mmol, 3.0 equiv) followed by the addition of 2-(4-chlorophenyl)acetyl chloride (0.52 g, 0.279 mmol, 1.2 equiv). The resulting reaction mixture was allowed to stir at RT for overnight. Product formation was confirmed by LCMS. The reaction mixture was concentrated under reduced pressure and water was added to the residue. The resulting solid was filtered off, washed with water (10 mL×4) and dried under vacuum to obtain trans-2-(4-chloro-3-fluorophenoxy)-N-(4-((2-(4-chlorophenyl)acetamido)methyl)cyclohexyl)acetamide (Compound 29—0.029 g, 26% Yield) as a white solid. LCMS 467.3 [M+H]⁺; ¹H NMR (400 MHz, DMSO-d₆) δ 8.04 (t, J=5.48 Hz, 1H), 7.92 (d, J=8.33 Hz, 1H), 7.48-7.56 (m, 1H), 7.30-7.42 (m, 2H), 7.17-7.29 (m, 2H), 7.05 (d, J=3.07 Hz, 1H), 6.76-6.89 (m, 1H), 4.48 (s, 2H), 3.52-3.61 (m, 1H), 3.40 (s, 2H), 2.89 (t, J=6.14 Hz, 2H), 1.55-1.83 (m, 4H), 1.32 (br. s., 1H), 1.11-1.23 (m, 2H), 0.87-1.01 (m, 2H).

### Example 30

### Synthesis of trans-2-(4-chloro-3-fluorophenoxy)-N-(4-((2-(3-chlorophenoxy)acetamido)methyl)cyclohexyl)acetamide

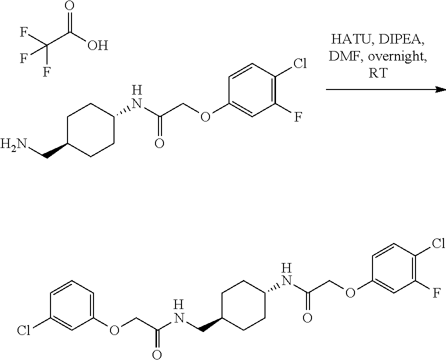

To a stirred solution of trans-N-(4-(aminomethyl)cyclohexyl)-2-(4-chloro-3-fluorophenoxy)acetamide 2,2,2-trifluoroacetate (0.100 g, 0.23 mmol, 1.0 equiv) in DMF (04 mL) was added HATU (0.177 g, 0.46 mmol, 2.0 equiv) at RT and stirred for 10 minutes. 2-(3-chlorophenoxy)acetic acid (0.044 g, 0.23 mmol, 1.0 equiv) was added followed by the addition of DIPEA (0.12 mL, 0.70 mmol, 3.0 equiv). The resulting reaction mixture was allowed to stir at RT for overnight. Product formation was confirmed by LCMS. The reaction mixture was diluted with water (50 mL) and the resulting solid was filtered off, washed with water (20 mL×4) and dried under vacuum. The crude product was enriched by flash chromatography (0-5% MeOH in DCM as an eluent) followed by the purification of reversed phase HPLC to trans-2-(4-chloro-3-fluorophenoxy)-N-(4-((2-(3-chlorophenoxy)acetamido)methyl)cyclohexyl)acetamide (Compound 30-0.055 g, 48% Yield) as a white solid. LCMS 483.4 [M+H]⁺; ¹H NMR (400 MHz, DMSO-d₆) δ 8.06-8.18 (m, 1H), 7.93 (d, J=8.33 Hz, 1H), 7.49 (t, J=8.77 Hz, 1H), 7.32 (t, J=8.33 Hz, 1H), 6.97-7.17 (m, 3H), 6.94 (d, J=8.33 Hz, 1H), 6.76-6.86 (m, 1H), 4.50 (d, J=17.54 Hz, 4H), 3.54 (br. s., 1H), 2.97 (t, J=6.58 Hz, 2H), 1.74 (d, J=9.21 Hz, 2H), 1.55-1.68 (m, 2H), 1.35 (br. s., 1H), 1.13-1.25 (m, 2H), 0.90-1.03 (m, 2H).

### Example 31

### Synthesis of trans-N-((4-(2-(4-chlorophenoxy)acetamido)cyclohexyl)methyl)-2-(4-chlorophenyl)-2-methylpropanamide

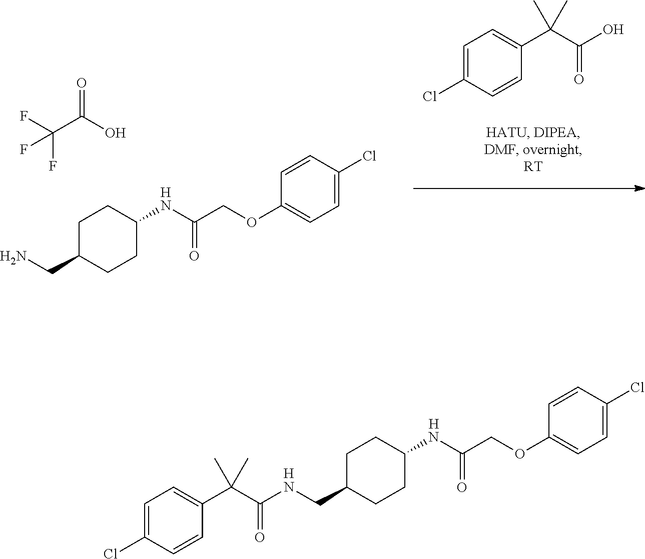

To a stirred solution of trans-N-(4-(aminomethyl)cyclohexyl)-2-(4-chlorophenoxy)acetamide (0.100 g, 0.24 mmol, 1.0 equiv) in DMF (05 mL) was added HATU (0.184 g, 0.48 mmol, 2.0 equiv) at RT and stirred for 10 minutes. 2-(4-chlorophenyl)-2-methylpropanoic acid (0.048 g, 0.24 mmol, 1.0 equiv) was added followed by the addition of DIPEA (0.12 mL, 0.72 mmol, 3.0 equiv). The resulting reaction mixture was allowed to stir at RT for overnight. Product formation was confirmed by LCMS. The reaction mixture was diluted with water (50 mL). The resulting solid was filtered off, washed with water (20 mL×4) and dried under vacuum to gives crude product which was purified by flash chromatography (0-5% MeOH in DCM as an eluent) to obtain trans-N-((4-(2-(4-chlorophenoxy)acetamido)cyclohexyl)methyl)-2-(4-chlorophenyl)-2-methylpropanamide (Compound 31—0.065 g, 56% Yield) a white solid. LCMS 477.4[M+H]⁺; ¹H NMR (400 MHz, DMSO-d₆) δ 7.87 (d, J=7.45 Hz, 1H), 7.22-7.45 (m, 7H), 6.96 (d, J=9.21 Hz, 2H), 4.43 (s, 2H), 3.52 (d, J=7.45 Hz, 1H), 2.85 (t, J=6.14 Hz, 2H), 1.70 (d, J=11.84 Hz, 2H), 1.55 (d, J=12.28 Hz, 1H), 1.43 (s, 6H), 1.33 (br. s., 1H), 1.10-1.21 (m, 1H), 0.75-0.94 (m, 2H).

### Example 32

### Synthesis of trans-N-((4-(2-(4-chloro-3-fluorophenoxy)acetamido)cyclohexyl)methyl)-2-(4-chlorophenyl)-2-methylpropanamide

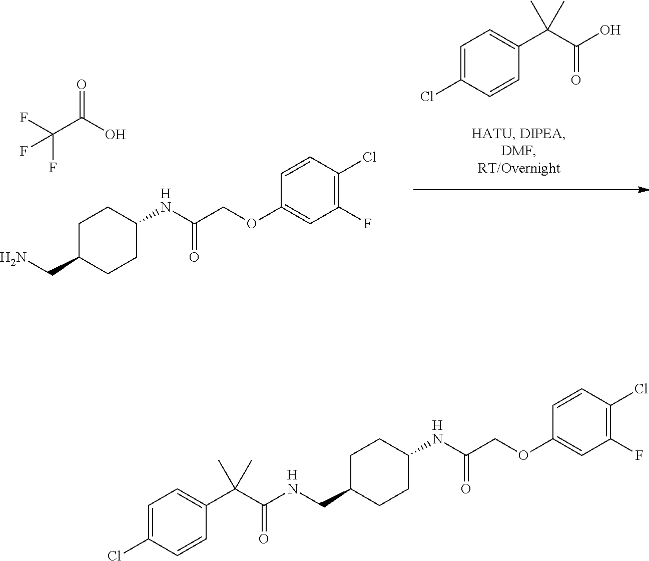

To a stirred solution of trans-N-(4-(aminomethyl)cyclohexyl)-2-(4-chloro-3-fluorophenoxy)acetamide 2,2,2-trifluoroacetate (0.100 g, 0.23 mmol, 1.0 equiv) in DMF (05 mL) was added HATU (0.177 g, 0.46 mmol, 2.0 equiv) at RT and stirred for 10 minutes. 2-(4-chlorophenyl)-2-methylpropanoic acid (0.046 g, 0.23 mmol, 1.0 equiv) was added followed by the addition of DIPEA (0.12 mL, 0.70 mmol, 3.0 equiv). The resulting reaction mixture was allowed to stir at RT for overnight. Product formation was confirmed by LCMS. The reaction mixture was diluted with water (50 mL). The resulting solid was filtered off, washed with water (20 mL×4) to gives crude product which was purified by flash chromatography (0-5% MeOH in DCM as an eluent) to obtain trans-N-((4-(2-(4-chloro-3-fluorophenoxy)acetamido)cyclohexyl)methyl)-2-(4-chlorophenyl)-2-methylpropanamide (Compound 32—0.055 g, 47% Yield) as a white solid. LCMS 495.4 [M+H]⁺; ¹H NMR (400 MHz, DMSO-d₆) δ 7.89 (d, J=7.89 Hz, 1H), 7.49 (t, J=8.77 Hz, 1H), 7.18-7.43 (m, 5H), 7.06 (dd, J=11.40, 2.63 Hz, 1H), 6.84 (d, J=9.21 Hz, 1H), 4.48 (s, 2H), 3.51 (d, J=8.33 Hz, 1H), 2.85 (t, J=5.92 Hz, 2H), 1.71 (d, J=10.09 Hz, 2H), 1.56 (d, J=11.40 Hz, 2H), 1.38-1.49 (m, 6H), 1.34 (br. s., 1H), 1.10-1.20 (m, 2H), 0.75-0.91 (m, 2H).

### Example 33

### Synthesis of trans-N-(4-(((6-chloro-1,2,3,4-tetrahydronaphthalen-2-yl)amino)methyl)cyclohexyl)-2-(4-chlorophenoxy)acetamide

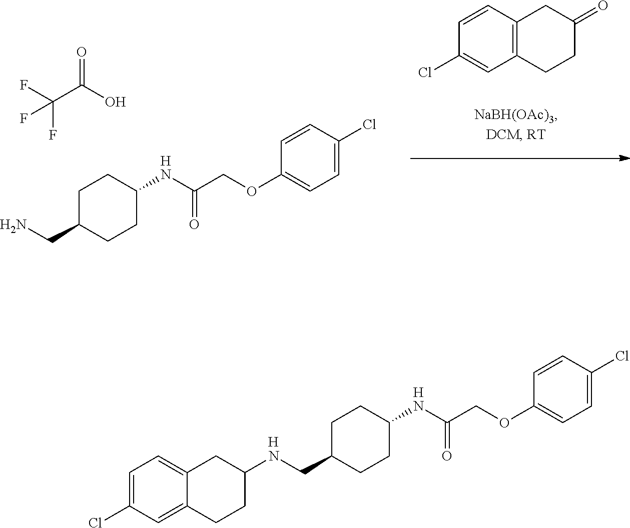

To a stirred solution of trans-N-(4-(aminomethyl)cyclohexyl)-2-(4-chlorophenoxy)acetamide (0.100 g, 0.243 mmol, 1.0 equiv) in DCM (5 mL) was added 6-chloro-3,4-dihydronaphthalen-2(1H)-one (0.088 g, 0.486 mmol, 2.0 equiv) at RT and stirred for 5 min. The resulting reaction mixture was cooled to 0° C. and NaBH(OAc)₃ (0.154 g, 0.729 mmol, 3.0 equiv) was added and allowed to stir at RT for overnight. Product formation was confirmed by TLC and LCMS. The reaction mixture was diluted with water (50 mL) and extracted with ethyl acetate (50 mL×2). The combined organic layer was washed with water (20 mL×2), dried over anhydrous sodium sulfate, filtered and concentrated under reduced pressure. The crude product was purified by reversed phase HPLC to obtain trans-N-(4-(((6-chloro-1,2,3,4-tetrahydronaphthalen-2-yl)amino)methyl)cyclohexyl)-2-(4-chlorophenoxy)acetamide (Compound 33—0.050 g, 45% Yield) as a white solid. LCMS 461.4 [M+H]⁺; ¹H NMR (400 MHz, DMSO-d₆) δ 8.23 (s, 1H), 7.91 (d, J=8.33 Hz, 1H), 7.29-7.39 (m, 2H), 7.04-7.21 (m, 3H), 6.91-7.00 (m, 2H), 4.44 (s, 2H), 3.58 (d, J=7.89 Hz, 2H), 2.90-3.04 (m, 2H), 2.75-2.90 (m, 2H), 2.62-2.75 (m, 2H), 1.98 (br. s., 1H), 1.79 (t, J=15.35 Hz, 4H), 1.53 (br. s., 2H), 1.37 (br. s., 1H), 1.11-1.33 (m, 3H), 0.85-1.06 (m, 3H).

### Example 34

### Synthesis of trans-6-chloro-N-(4-(2-(4-chlorophenoxy)acetamido)cyclohexyl)-3,4-dihydro-2H-benzo[b][1,4]oxazine-3-carboxamide

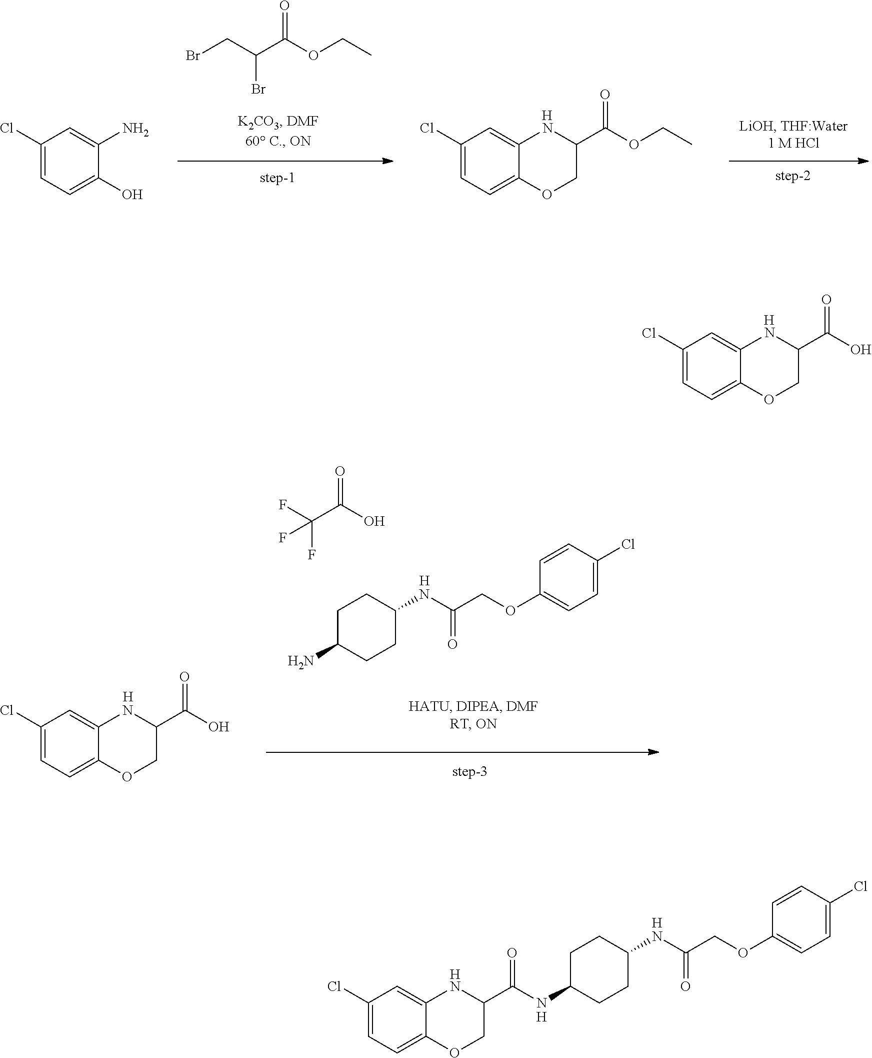

### Step 1—Synthesis of ethyl 6-chloro-3,4-dihydro-2H-benzo[b][1,4]oxazine-3-carboxylate

To a stirred solution of 2-amino-4-chlorophenol (5.00 g, 34.85 mmol, 1.0 equiv) in DMF (50 mL) was added K₂CO3 (24.00 g, 174.30 mmol, 5.0 equiv) followed by the addition of 4 ethyl 2,3-dibromopropanoate (11.80 g, 45.29 mmol, 1.3 equiv) at RT. The resulting reaction mixture was allowed to stir at 60° C. for overnight. Product formation was confirmed by TLC and LCMS. The reaction mixture was diluted with water (100 mL) and extracted with ethyl acetate (250 mL×2). Combined organic layer was washed with water (100 mL×4), dried over anhydrous Na₂SO4 and concentrated under reduced pressure. The crude product was purified by flash chromatography (0-20% Ethyl acetate in Hexane as an eluent) to obtain ethyl 6-chloro-3,4-dihydro-2H-benzo[b][1,4]oxazine-2-carboxylate (2.5 g 30% Yield) and ethyl 6-chloro-3,4-dihydro-2H-benzo[b][1,4]oxazine-3-carboxylate (0.500 g, 6% Yield) as alight brown solid. LCMS 241.9 [M+H]⁺; ¹H NMR (400 MHz, DMSO-d₆) δ 6.71 (d, J=2.19 Hz, 1H), 6.54-6.68 (m, 2H), 6.47 (dd, J=8.55, 2.41 Hz, 1H), 4.34-4.44 (m, 1H), 4.26 (q, J=2.78 Hz, 1H), 4.02-4.16 (m, 3H), 1.19 (t, J=7.02 Hz, 3H).

### Step 2—Synthesis of 6-chloro-3,4-dihydro-2H-benzo[b][1,4]oxazine-3-carboxylic acid

To a stirred solution of ethyl 6-chloro-3,4-dihydro-2H-benzo[b][1,4]oxazine-3-carboxylate (0.200 g, 0.826 mmol, 1.0 equiv) in THF:Water (5:5 mL), was added LiOH.H2O (0.105 g, 2.479 mmol, 3.0 equiv). The resulting reaction mixture was stirred at RT for overnight. Reaction was monitored by LCMS. After completion of reaction, the reaction mixture was quenched with 1 M HCl (25 mL) and extracted with ethyl acetate (50 mL×2). Combined organic layer was dried over anhydrous sodium sulfate and concentrated under reduced pressure to obtain 6-chloro-3,4-dihydro-2H-benzo[b][1,4]oxazine-3-carboxylic acid (0.200 g, Quant. Yield) as alight brown solid. LCMS 213.8 [M+H]⁺.

### Step 3—Synthesis of trans-6-chloro-N-(4-(2-(4-chlorophenoxy)acetamido)cyclohexyl)-3,4-dihydro-2H-benzo[b][1,4]oxazine-3-carboxamide

To a stirred solution of trans-N-(4-aminocyclohexyl)-2-(4-chlorophenoxy)acetamide 2,2,2-trifluoroacetate (0.372 g, 0.938 mmol, 1.0 equiv) and 6-chloro-3,4-dihydro-2H-benzo[b][1,4]oxazine-3-carboxylic acid (0.200 g, 0.938 mmol, 1.0 equiv) in DMF (5 mL) was added HATU (0.536 g, 1.410 mmol, 1.5 equiv) followed by the addition of DIPEA (0.5 mL, 2.816 mmol, 3.0 equiv). The resulting reaction mixture was allowed to stir for overnight at RT. Product formation was confirmed by LCMS. After the completion of reaction the reaction mixture was diluted with water (50 mL) and extracted with ethyl acetate (50 mL×2). Combined organic layer was washed with water (20 mL×4), dried over anhydrous sodium sulfate and concentrated under reduced pressure. The resulting solid was washed with MeOH and dried under vacuum to obtain trans-6-chloro-N-(4-(2-(4-chlorophenoxy)acetamido)cyclohexyl)-3,4-dihydro-2H-benzo[b][1,4]oxazine-3-carboxamide (Compound 34—0.060 g, 14% Yield) as an off-white solid. LCMS 478.3 [M+H]⁺; ¹H NMR (400 MHz, DMSO-d₆) δ 7.93 (d, J=7.89 Hz, 1H), 7.77 (d, J=7.89 Hz, 1H), 7.34 (m, J=8.77 Hz, 2H), 6.91-7.04 (m, 2H), 6.60-6.76 (m, 2H), 6.47 (dd, J=8.33, 2.63 Hz, 1H), 6.39 (d, J=3.51 Hz, 1H), 4.44 (s, 2H), 4.16 (dd, J=10.52, 4.39 Hz, 1H), 3.95-4.08 (m, 1H), 3.81-3.95 (m, 1H), 3.54 (d, J=12.72 Hz, 2H), 1.74 (d, J=9.21 Hz, 4H), 1.26-1.39 (m, 4H).

### Example 35

### Synthesis of trans-6-chloro-N-(4-(2-(4-chlorophenoxy) acetamido) cyclohexyl)-4-methyl-3,4-dihydro-2H-benzo[b][1,4]oxazine-2-carboxamide

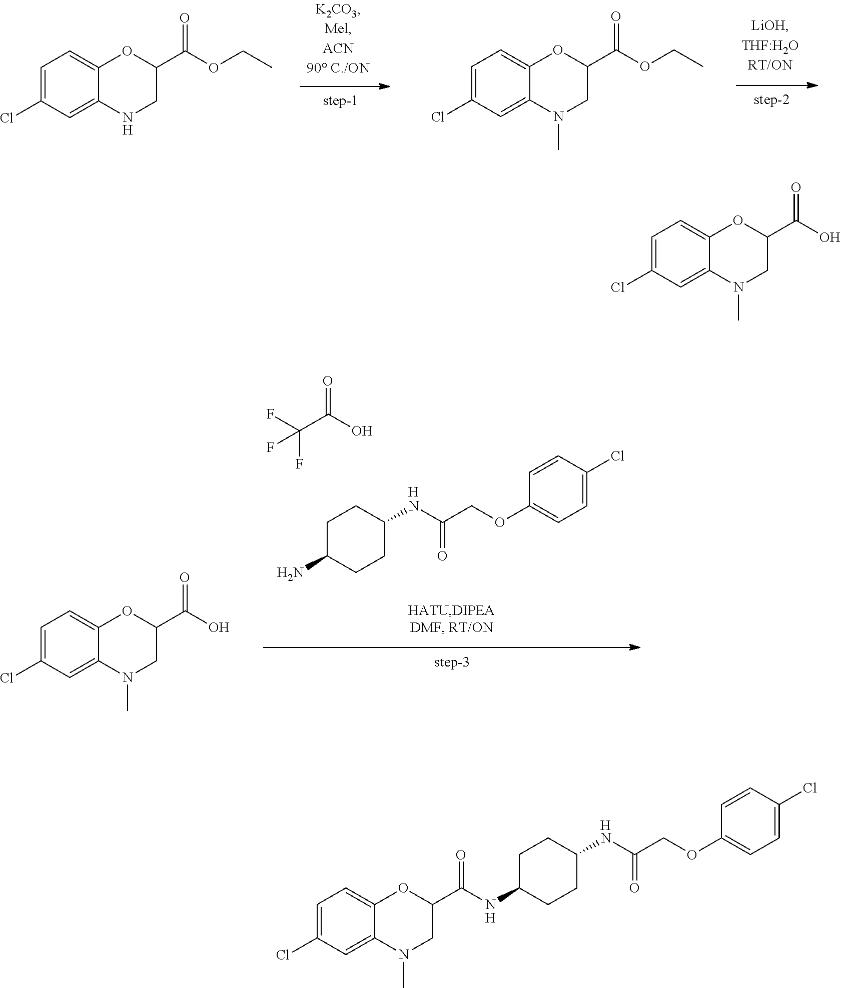

### Step 1—Synthesis of ethyl 6-chloro-4-methyl-3,4-dihydro-2H-benzo[b][1,4]oxazine-2-carboxylate

To the solution of ethyl 6-chloro-3,4-dihydro-2H-benzo[b][1,4]oxazine-2-carboxylate (1.0 g, 4.1 mmol, 1.0 equiv) in ACN (10 mL), was added MeI (0.5 mL, 8.2 mmol, 2.0 equiv), and K₂CO3 (1.14 g, 8.2 mmol, 2.0 equiv). The resulting reaction mixture was heated at 90° C. for overnight. Product formation was confirmed by 1H NMR. After completion of reaction the reaction mixture was diluted with water (100 mL) and extracted with ethyl acetate (100 mL×2). Combined organic layer was dried over anhydrous Na₂SO₄ and concentrated under reduced pressure to obtain crude material which was purified by flash chromatography (0-20% Ethyl acetate in Hexane as an eluent) to obtain ethyl 6-chloro-4-methyl-3,4-dihydro-2H-benzo[b][1,4]oxazine-2-carboxylate (0.600 g, 57% Yield), as a yellow semi solid. ¹H NMR (400 MHz, DMSO-d₆) δ 6.77 (d, J=8.33 Hz, 1H) 6.70 (d, J=2.19 Hz, 1H) 6.62 (dd, J=8.33, 2.63 Hz, 1H) 5.07 (t, J=3.51 Hz, 1H) 4.05-4.16 (m, 2H) 3.40 (dd, J=11.84, 3.51 Hz, 2H) 2.82 (s, 3H) 1.17 (t, J=7.02 Hz, 3H).

### Step 2—Synthesis of 6-chloro-4-methyl-3,4-dihydro-2H-benzo[b][1,4]oxazine-2-carboxylic acid

To a stirred solution of ethyl 6-chloro-4-methyl-3,4-dihydro-2H-benzo[b][1,4]oxazine-2-carboxylate (0.600 g, 2.3 mmol, 1.0 equiv) in THF (10 mL) and water (5 mL), was added LiOH (0.113 g, 4.7 mmol, 2.0 equiv). The mixture was allowed to stir at RT for overnight. Product formation was confirmed by LCMS and ¹H NMR Spectroscopy. The reaction mixture was concentrated and lypholize to obtain 6-chloro-4-methyl-3,4-dihydro-2H-benzo[b][1,4]oxazine-2-carboxylic acid (0.200 g, 38% Yield) as an off-white solid. LCMS 227.9 [M+H]⁺; ¹H NMR (400 MHz, DMSO-d₆) δ 13.11 (br. s., 1H) 6.67-6.79 (m, 2H) 6.57-6.65 (m, 1H) 4.95 (t, J=3.73 Hz, 1H) 3.36-3.49 (m, 2H) 2.82 (s, 3H).

### Step 3—Synthesis of trans-6-chloro-N-(4-(2-(4-chlorophenoxy) acetamido) cyclohexyl)-4-methyl-3,4-dihydro-2H-benzo[b][1,4]oxazine-2-carboxamide

To a stirred solution of 6-chloro-4-methyl-3,4-dihydro-2H-benzo[b][1,4]oxazine-2-carboxylic acid (0.100 g, 0.44 mmol, 1 equiv) in DMF (05 mL), was added HATU (0.334 g, 0.88 mmol, 2.0 equiv) and trans-N-(4-aminocyclohexyl)-2-(4-chlorophenoxy)acetamide 2,2,2-trifluoroacetate (0.175 g, 0.44 mmol, 1.0 equiv). The reaction mixture was allowed to stir at RT for 10 min. DIPEA (0.22 mL, 1.32 mmol, 3.0 equiv) was added and the reaction mixture was allowed to stir for overnight at RT. Progress of the reaction was monitored by ¹H NMR and LCMS. After completion of the reaction, the reaction mixture was diluted with water (100 mL) and precipitated solid was filtered off and dried under vacuum to obtain trans-6-chloro-N-(4-(2-(4-chlorophenoxy)acetamido)cyclohexyl)-4-methyl-3,4-dihydro-2H-benzo[b][1,4]oxazine-2-carboxamide (Compound 83—0.140 g, 64% Yield) as an off-white solid. LCMS: 492.2 [M+H]⁺; ¹H NMR (400 MHz, DMSO-d₆) δ 7.95 (d, J=8.33 Hz, 1H) 7.86 (d, J=8.33 Hz, 1H) 7.34 (m, J=8.77 Hz, 2H) 6.96 (m, J=8.77 Hz, 2H) 6.81 (d, J=8.77 Hz, 1H) 6.69 (s, 1H) 6.61 (d, J=8.77 Hz, 1H) 4.62 (d, J=4.82 Hz, 1H) 4.44 (s, 2H) 3.57 (br. s., 2H) 3.41 (br. s., 1H) 3.18 (dd, J=11.62, 7.67 Hz, 1H) 2.83 (s, 3H) 1.76 (br. s., 3H) 1.69 (br. s., 1H) 1.22-1.42 (m, 4H).

### Example 36

### Synthesis of trans-2-(4-chlorophenoxy)-N-(4-((2-(4-chlorophenyl)acetamido)methyl)cyclohexyl)acetamide

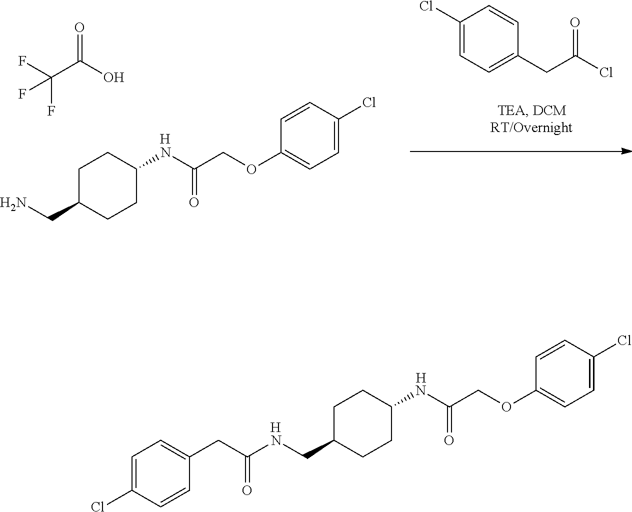

To a stirred solution of trans-N-(4-(aminomethyl)cyclohexyl)-2-(4-chlorophenoxy)acetamide 2,2,2-trifluoroacetate (0.100 g, 0.243 mmol, 1.0 equiv) in DCM (05 ml) was added 2-(4-chlorophenyl)acetyl chloride (0.55 g, 0.292 mmol, 1.2 equiv) followed by the addition of TEA (0.1 mL, 0.731 mmol, 3.0 equiv). The resulting reaction mixture was allowed to stir at RT for overnight. Product formation was confirmed by LCMS. The reaction mixture was concentrated by vaccume.& The reaction mixture was diluted with water (50 ml) The resulting solid was filtered off, washed with water (10 mL×4) to obtain trans-2-(4-chlorophenoxy)-N-(4-((2-(4-chlorophenyl)acetamido)methyl)cyclohexyl)acetamide (Compound 84—0.005 g, 5% Yield) as a white solid. LCMS 449.3 [M+H]⁺; ¹H NMR (400 MHz, DMSO-d₆) δ 8.04 (t, J=5.48 Hz, 1H) 7.92 (d, J=8.33 Hz, 1H) 7.48-7.56 (m, 1H) 7.30-7.42 (m, 2H) 7.17-7.29 (m, 3H) 7.05 (d, J=3.07 Hz, 1H) 6.76-6.89 (m, 1H) 4.48 (s, 2H) 3.52-3.61 (m, 2H) 3.40 (s, 1H) 2.89 (t, J=6.14 Hz, 2H) 1.55-1.83 (m, 4H) 1.32 (br. s., 1H) 1.11-1.23 (m, 2H) 0.87-1.01 (m, 2H).

### Example 37

### Chiral Resolution of trans-6-chloro-N-(4-(2-(4-chlorophenoxy)acetamido)cyclohexyl)-4-methyl-3,4-dihydro-2H-benzo[b][1,4]oxazine-2-carboxamide

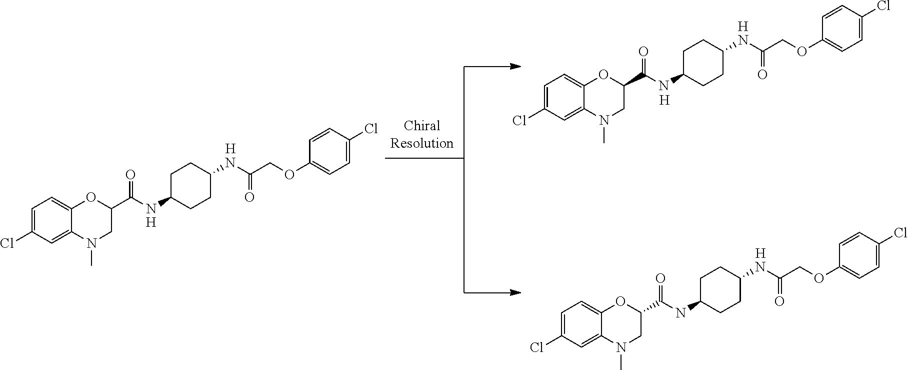

The enantiomers, trans-(R)-6-chloro-N-(4-(2-(4-chlorophenoxy)acetamido)cyclohexyl)-4-methyl-3,4-dihydro-2H-benzo[b][1,4]oxazine-2-carboxamide (Compound 85—[α]_{D}²⁰=not available, elution time: 8.44 min) and trans-(S)-6-chloro-N-(4-(2-(4-chlorophenoxy)acetamido)cyclohexyl)-4-methyl-3,4-dihydro-2H-benzo[b][1,4]oxazine-2-carboxamide (Compound 86—[α]_{D}²⁰=not available, elution time: 16.52 min), were separated by chiral SFC (Chiralcel-ODH, 20×250 mm, 5 μm). Isocratic program with analytical grade liquid carbon dioxide and HPLC grade MeOH. LCMS: 492.3 [M+H]⁺; ¹H NMR (400 MHz, DMSO-d) δ7.94 (d, J=8.33 Hz, 1H) 7.85 (d, J=8.33 Hz, 1H) 7.34 (m, J=9.21 Hz, 2H) 6.96 (m, J=9.21 Hz, 2H) 6.81 (d, J=8.77 Hz, 1H) 6.70 (d, J=2.19 Hz, 1H) 6.61 (dd, J=8.55, 2.41 Hz, 1H) 4.62 (dd, J=7.24, 2.85 Hz, 1H) 4.44 (s, 2H) 3.57 (br. s., 2H) 3.39 (d, J=2.63 Hz, 2H) 3.18 (dd, J=11.84, 7.45 Hz, 2H) 2.83 (s, 3H) 1.76-1.69 (in, 4H) 1.34 (d, J=9.65 Hz, 4H).

### Example 38

### Synthesis of trans-6-chloro-N-(4-(2-(4-chlorophenoxy)acetamido)cyclohexyl)-4-ethyl-3,4-dihydro-2H-benzo[b][1,4]oxazine-2-carboxamide

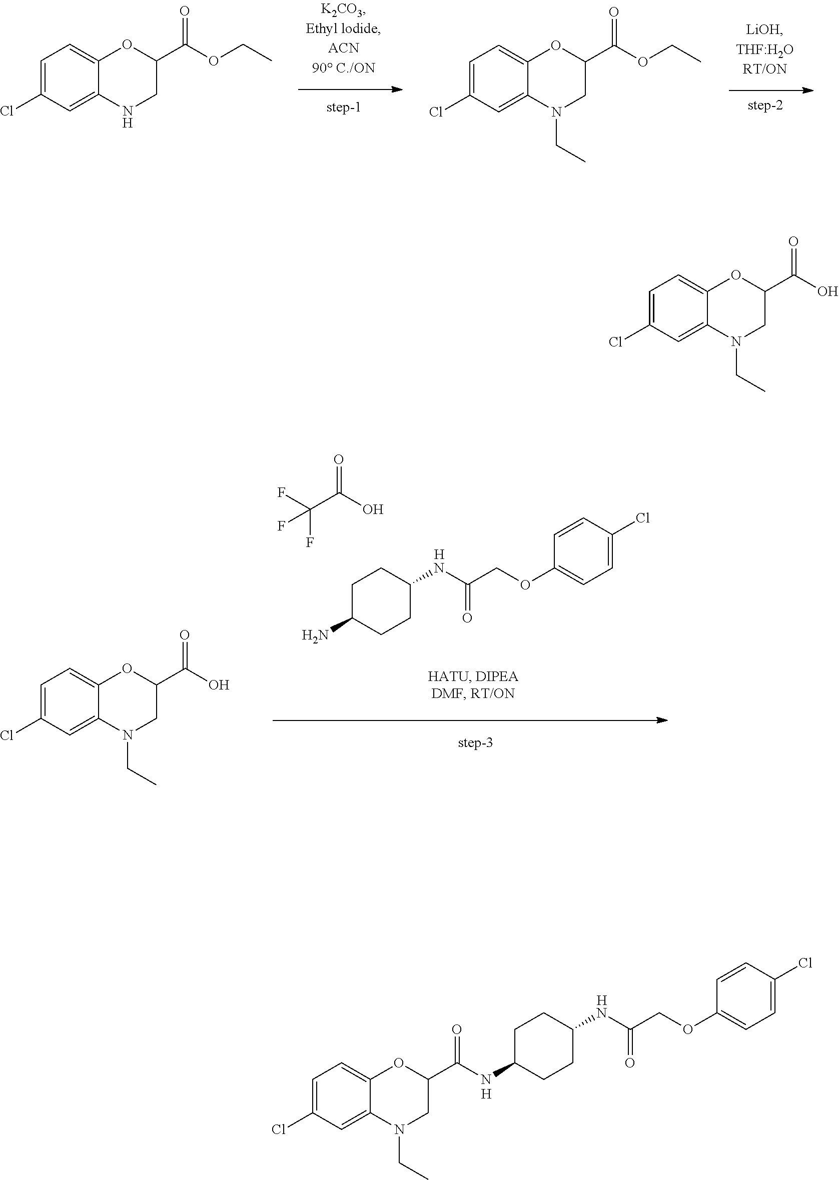

### Step 1—Synthesis of ethyl 6-chloro-4-ethyl-3,4-dihydro-2H-benzo[b][1,4]oxazine-2-carboxylate

To the solution of ethyl 6-chloro-3,4-dihydro-2H-benzo[b][1,4]oxazine-2-carboxylate (0.500 g, 2.07 mmol, 1.0 equiv) in DMF (05 mL), was added ethyl iodide (0.323 g, 2.07 mmol, 1.0 equiv), and NaH (0.100 g, 4.14 mmol, 2.0 equiv). The resulting reaction mixture was stirred at RT for 2 hr. Product formation was confirmed by 1H NMR. After completion of reaction the reaction mixture was diluted with water (100 mL) and extracted with ethyl acetate (100 mL×2). Combined organic layer was dried over anhydrous Na₂SO₄ and concentrated under reduced pressure to obtain crude material which was purified by flash chromatography (0-20% Ethyl acetate in hexane as an eluent) to obtain ethyl 6-chloro-4-ethyl-3,4-dihydro-2H-benzo[b][1,4]oxazine-2-carboxylate (0.250 g, 50% Yield), as a yellow semi solid. ¹H NMR (400 MHz, DMSO-d₆) δ 6.67-6.82 (m, 2H) 6.57 (dd, J=8.55, 2.41 Hz, 1H) 5.04 (t, J=3.51 Hz, 1H) 4.09-4.19 (m, 2H) 3.43 (dd, J=11.62, 3.73 Hz, 2H) 3.29 (d, J=7.45 Hz, 2H) 1.17 (t, J=7.02 Hz, 3H) 1.02 (t, J=7.02 Hz, 3H).

### Step 2—Synthesis of 6-chloro-4-ethyl-3,4-dihydro-2H-benzo[b][1,4]oxazine-2-carboxylic acid

To a stirred solution of ethyl 6-chloro-4-ethyl-3,4-dihydro-2H-benzo[b][1,4]oxazine-2-carboxylate (0.250 g, 0.92 mmol, 1.0 equiv) in THF (05 mL) and water (03 mL), was added LiOH (0.045 g, 1.85 mmol, 2.0 equiv). The mixture was allowed to stir at RT for overnight. Product formation was confirmed by LCMS and ¹H NMR Spectroscopy. The reaction mixture was acidified by using 1N HCl, precipitated solid was filtered off and dried under vacuum to obtain 6-chloro-4-ethyl-3,4-dihydro-2H-benzo[b][1,4]oxazine-2-carboxylic acid (0.100 g, 45% Yield) as an off-white solid. LCMS 242.0 [M+H]⁺; ¹H NMR (400 MHz, DMSO-d₆) δ 13.09 (br. s., 1H) 6.66-6.79 (m, 2H) 6.50-6.60 (m, 1H) 4.90 (br. s., 1H) 3.42 (br. s., 2H) 3.26 (br. s., 2H) 1.03 (t, J=7.02 Hz, 3H).

### Step 3—Synthesis of trans-6-chloro-N-(4-(2-(4-chlorophenoxy)acetamido)cyclohexyl)-4-ethyl-3,4-dihydro-2H-benzo[b][1,4]oxazine-2-carboxamide

To a stirred solution of 6-chloro-4-ethyl-3,4-dihydro-2H-benzo[b][1,4]oxazine-2-carboxylic acid (0.100 g, 0.41 mmol, 1 equiv) in DMF (05 mL), was added HATU (0.311 g, 0.82 mmol, 2.0 equiv) and trans-N-(4-aminocyclohexyl)-2-(4-chlorophenoxy)acetamide 2,2,2-trifluoroacetate (0.164 g, 0.41 mmol, 1.0 equiv). The reaction mixture was allowed to stir at RT for 10 min. DIPEA (0.21 mL, 1.23 mmol, 3.0 equiv) was added and the reaction mixture was allowed to stir for overnight at RT. Progress of the reaction was monitored by ¹H NMR and LCMS. After completion of the reaction, the reaction mixture was diluted with water (100 mL) and precipitated solid was filtered off, dried under vacuum to obtain trans-6-chloro-N-(4-(2-(4-chlorophenoxy)acetamido)cyclohexyl)-4-ethyl-3,4-dihydro-2H-benzo[b][1,4]oxazine-2-carboxamide (Compound 87—0.140 g, 67% Yield) as an off-white solid. LCMS: 506.5 [M+H]⁺; ¹H NMR (400 MHz, DMSO-d₆) δ 7.94 (d, J=7.89 Hz, 1H) 7.85 (d, J=7.89 Hz, 1H) 7.34 (m, J=8.77 Hz, 2H) 6.96 (m, J=9.21 Hz, 2H) 6.81 (d, J=8.33 Hz, 1H) 6.72 (d, J=2.19 Hz, 1H) 6.50-6.60 (m, 1H) 4.50-4.60 (m, 1H) 4.45 (s, 2H) 3.57 (br. s., 2H) 3.36-3.45 (m, 2H) 3.19-3.27 (m, 2H) 1.76 (br. s., 2H) 1.70 (br. s., 2H) 1.34 (d, J=8.77 Hz, 4H) 1.03 (t, J=7.02 Hz, 3H).

### Example 39

### Chiral Resolution of trans-6-chloro-N-(4-(2-(4-chlorophenoxy)acetamido)cyclohexyl)-4-ethyl-3,4-dihydro-2H-benzo[b][1,4]oxazine-2-carboxamide

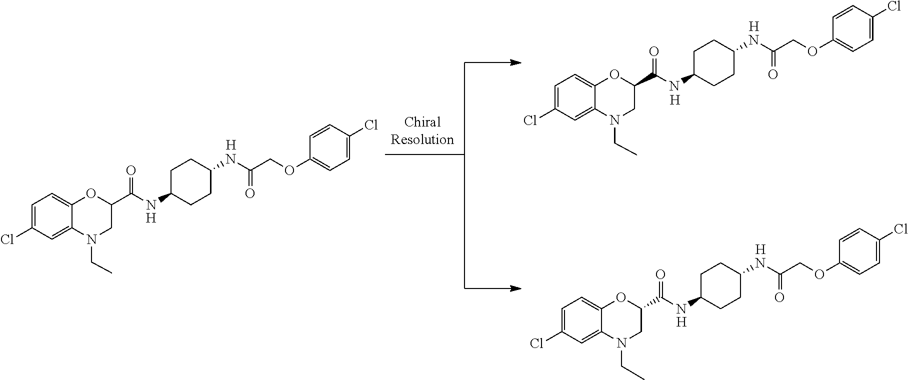

The enantiomers, trans-(R)-6-chloro-N-(4-(2-(4-chlorophenoxy)acetamido)cyclohexyl)-4-ethyl-3,4-dihydro-2H-benzo[b][1,4]oxazine-2-carboxamide (Compound 88—[α]_{D}²⁰=not available, elution time: 5.26 min) and trans-(S)-6-chloro-N-(4-(2-(4-chlorophenoxy)acetamido)cyclohexyl)-4-ethyl-3,4-dihydro-2H-benzo[b] [1,4]oxazine-2-carboxamide (Compound 89 —[α]_{D}²⁰=not available, elution time: 7.94 min), were separated by chiral SFC (Chiralpak-IB, 20×250 mm, 5 μm). Isocratic program with analytical grade liquid carbon dioxide and HPLC grade MeOH. LCMS: 506.4 [M+H]⁺; ¹H NMR (400 MHz, DMSO-d₆) δ ppm 7.95 (d, J=7.89 Hz, 1H) 7.86 (d, J=8.33 Hz, 1H) 7.24-7.39 (m, 2H) 6.89-7.01 (m, 2H) 6.81 (d, J=8.77 Hz, 1H) 6.72 (d, J=2.19 Hz, 1H) 6.55 (dd, J=8.55, 2.41 Hz, 1H) 4.54 (dd, J=7.45, 3.07 Hz, 1H) 4.45 (s, 2H) 3.57 (br. s., 2H) 3.36-3.45 (m, 2H) 3.17-3.29 (m, 2H) 1.76-1.70 (m, 4H) 1.23-1.44 (m, 4H).

### Example 40

### Synthesis of trans-2-(4-chloro-2-methoxyphenoxy)-N-(4-(2-(4-chlorophenoxy)acetamido)cyclohexyl)acetamide

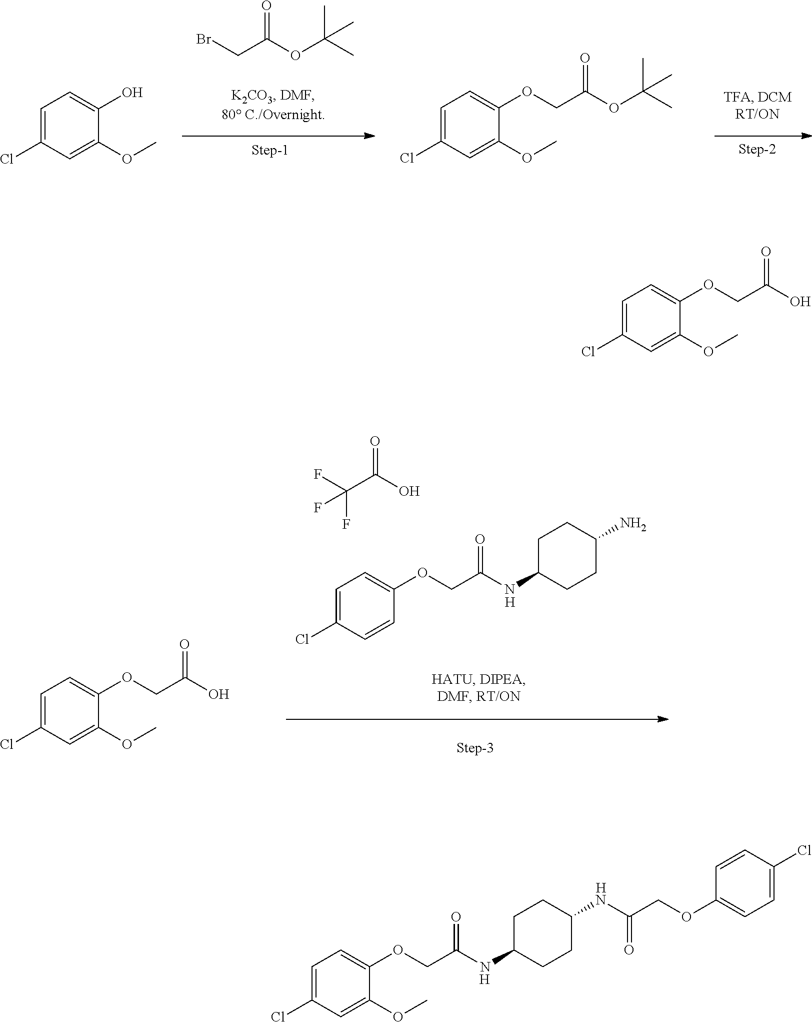

### Step 1—Synthesis of tert-butyl 2-(4-chloro-2-methoxyphenoxy)acetate

To a solution of 4-chloro-2-methoxyphenol (0.500 g, 3.1 mmol, 1.0 equiv) in DMF (05 mL) was added tert-butyl 2-bromoacetate (0.740 g, 3.7 mmol, 1.2 equiv), K₂CO3 (0.855 g, 6.2 mmol, 2.0 equiv). The resulting reaction mixture was heated at 80° C. for overnight. Product formation was confirmed by ¹H NMR. After completion of reaction, the mixture was diluted with water (50 mL) and extracted with ethyl acetate (100 mL×2). Combined organic extracts were washed with water (50 mL×4), dried over anhydrous Na₂SO₄ and concentrated to obtain tert-butyl 2-(4-chloro-2-methoxyphenoxy)acetate (0.400 g, 48% Yield) as an oil. ¹H NMR (400 MHz, DMSO-d₆) δ 7.04 (d, J=2.63 Hz, 1H) 6.85-6.94 (m, 2H) 4.64 (s, 2H) 3.33 (s, 3H) 1.41 (s, 9H).

### Step 2—Synthesis of 2-(4-chloro-2-methoxyphenoxy) acetic acid

To a stirred solution of tert-butyl 2-(4-chloro-2-methoxyphenoxy)acetate (0.400 g, 1.4 mmol, 1.0 equiv) in DCM (10 mL), was added TFA (03 mL) and the resultant reaction mixture was stirred at RT for 1 h under nitrogen atmosphere. Reaction was monitored by TLC and LCMS. After completion of reaction, the reaction mixture was concentrated under reduced pressure. Crude compound was crystallized in hexane (10 mL) and diethyl ether, filtered off and dried under vacuum to obtain 2-(4-chloro-2-methoxyphenoxy)acetic acid (0.300 g, 99% Yield) as a brown solid. ¹H NMR (400 MHz, DMSO-d₆) δ 12.98 (br. s., 1H) 7.03 (br. s., 1H) 6.79-6.92 (m, 2H) 4.66 (s, 2H) 3.33 (s, 3H).

### Step 3—Synthesis of trans-2-(4-chloro-2-methoxyphenoxy)-N-(-4-(2-(4-chlorophenoxy)acetamido)cyclohexyl)acetamide

To a stirred solution of 2-(4-chloro-2-methoxyphenoxy)acetic acid (0.100 g, 0.46 mmol, 1.0 equiv) in DMF (05 mL) was added HATU (0.350 g, 0.92 mmol, 2.0 equiv) at RT and stirred for 10 minutes. Then trans-N-(4-aminocyclohexyl)-2-(4-chlorophenoxy)acetamide 2,2,2-trifluoroacetate (0.183 g, 0.46 mmol, 1.0 equiv) was added followed by the addition of DIPEA (0.23 mL, 1.38 mmol, 3.0 equiv). The resulting reaction mixture was allowed to stir at RT for overnight. Product formation was confirmed by LCMS. The reaction mixture was diluted with water (50 mL). The resulting solid was filtered off, washed with water (20 mL×4) and dried under vacuum to obtain trans-2-(4-chloro-2-methoxyphenoxy)-N-(4-(2-(4-chlorophenoxy)acetamido)cyclohexyl)acetamide (Compound 90—0.90 g, 40% Yield) as an off white solid. LCMS 481.1 [M+H]⁺; ¹H NMR (400 MHz, DMSO-d₆) δ 7.95 (d, J=7.89 Hz, 1H) 7.82 (d, J=7.89 Hz, 1H) 7.34 (d, J=8.77 Hz, 2H) 7.06 (s, 1H) 6.76-7.01 (m, 4H) 4.44 (d, J=6.58 Hz, 4H) 3.81 (s, 3H) 3.57 (br. s., 2H) 1.76 (d, J=8.77 Hz, 4H) 1.32 (q, J=8.48 Hz, 4H).

### Example 41

### Synthesis of trans-2-(4-chloro-2-methylphenoxy)-N-(4-(2-(4-chlorophenoxy)acetamido)cyclohexyl)acetamide

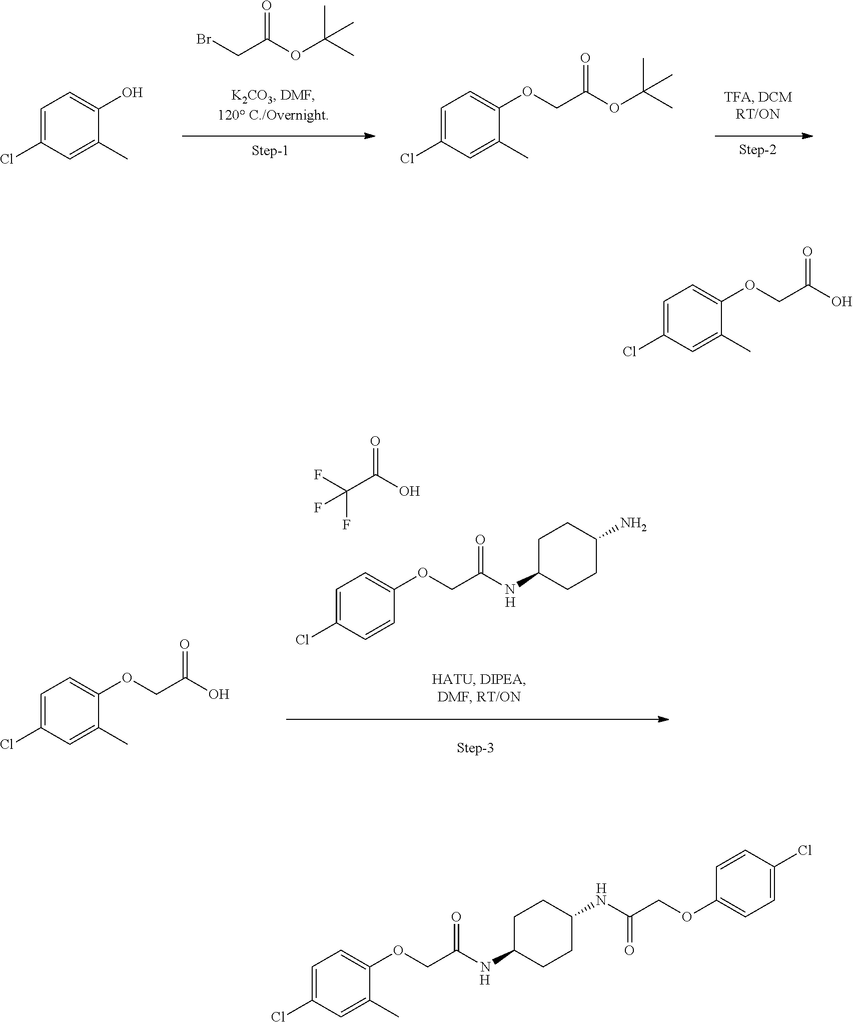

### Step 1—Synthesis of tert-butyl 2-(4-chloro-2-methylphenoxy)acetate

To a solution of 4-chloro-2-methylphenol (0.250 g, 1.760 mmol, 1.0 equiv) in DMF (05 mL) was added tert-butyl 2-bromoacetate (0.343 g, 1.760 mmol, 1.0 equiv), K₂CO3 (0.485 g, 3.521 mmol, 2.0 equiv). The resulting reaction mixture was heated at 120° C. for overnight. Product formation was confirmed by ¹H NMR. The reaction mixture was diluted with water (50 mL) and extracted with EtOAc (100 mL×2). The combined organic layer was washed with water (50 mL), brine solution (50 mL×2), dried over anhydrous sodium sulphate and concentrated under reduced pressure to obtain tert-butyl 2-(4-chloro-2-methylphenoxy)acetate (0.200 g, 45% Yield) as an oil. ¹H NMR (400 MHz, DMSO-d₆) δ 7.04-7.26 (m, 2H) 6.83 (d, J=8.77 Hz, 1H) 4.69 (s, 2H) 2.18 (s, 3H) 1.29-1.55 (m, 9H).

### Step 2—Synthesis of 2-(4-chloro-2-methylphenoxy)acetic acid

To a stirred solution of tert-butyl 2-(4-chloro-2-methylphenoxy)acetate (0.200 g, 0.781 mmol, 1.0 equiv) in DCM (5 mL), was added TFA (0.5 mL) and the resultant reaction mixture was stirred at RT for overnight under nitrogen atmosphere. Reaction was monitored by TLC and LCMS. After completion of reaction, the reaction mixture was concentrated under reduced pressure. Crude compound was crystallized in hexane (10 mL) and diethyl ether, filtered off and dried under vacuum to obtain to obtain 2-(4-chloro-2-methylphenoxy)acetic acid (0.150 g, 96% Yield) as a brown solid. ¹H NMR (400 MHz, DMSO-d₆) δ 13.02 (br. s., 1H) 7.06-7.29 (m, 2H) 6.84 (d, J=8.77 Hz, 1H) 4.71 (s, 2H) 2.18 (s, 3H).

### Step 3: Synthesis of trans-2-(4-chloro-2-methylphenoxy)-N-(-4-(2-(4-chlorophenoxy)acetamido)cyclohexyl)acetamide

To a stirred solution of 2-(4-chloro-2-methylphenoxy)acetic acid (0.100 g, 0.252 mmol, 1.0 equiv) in DMF (04 mL) was added HATU (0.192 g, 0.505 mmol, 2.0 equiv) at RT and stirred for 10 minutes. Then trans-N-(-4-aminocyclohexyl)-2-(4-chlorophenoxy)acetamide 2,2,2-trifluoroacetate (0.050 g, 0.252 mmol, 1.0 equiv) was added followed by the addition of DIPEA (0.13 mL, 0.757 mmol, 3.0 equiv). The resulting reaction mixture was allowed to stir at RT for overnight. Product formation was confirmed by LCMS. The reaction mixture was diluted with water (20 mL×4). The resulting solid was filtered off, washed with water (20 mL) and dried under vacuum to obtain trans-2-(4-chloro-2-methylphenoxy)-N-(4-(2-(4-chlorophenoxy)acetamido)cyclohexyl)acetamide (Compound 91—0.070 g, 60% Yield) as an off white solid. LCMS 465.4 [M+H]⁺; ¹H NMR (400 MHz, DMSO-d₆) δ 7.95 (d, J=7.89 Hz, 1H) 7.83 (d, J=7.89 Hz, 1H) 7.34 (m, J=8.77 Hz, 2H) 7.16-7.29 (m, 2H) 6.96 (m, J=8.77 Hz, 2H) 6.81 (d, J=8.77 Hz, 1H) 4.46 (d, J=7.89 Hz, 4H) 3.58 (br. s., 2H) 2.20 (s, 3H) 1.77 (d, J=8.33 Hz, 4H) 1.33 (br. s., 4H).

### Example 42

### Synthesis of trans-5-chloro-2-(2-(4-(2-(4-chlorophenoxy)acetamido)cyclohexyl)amino)-2-oxoethoxy)benzamide

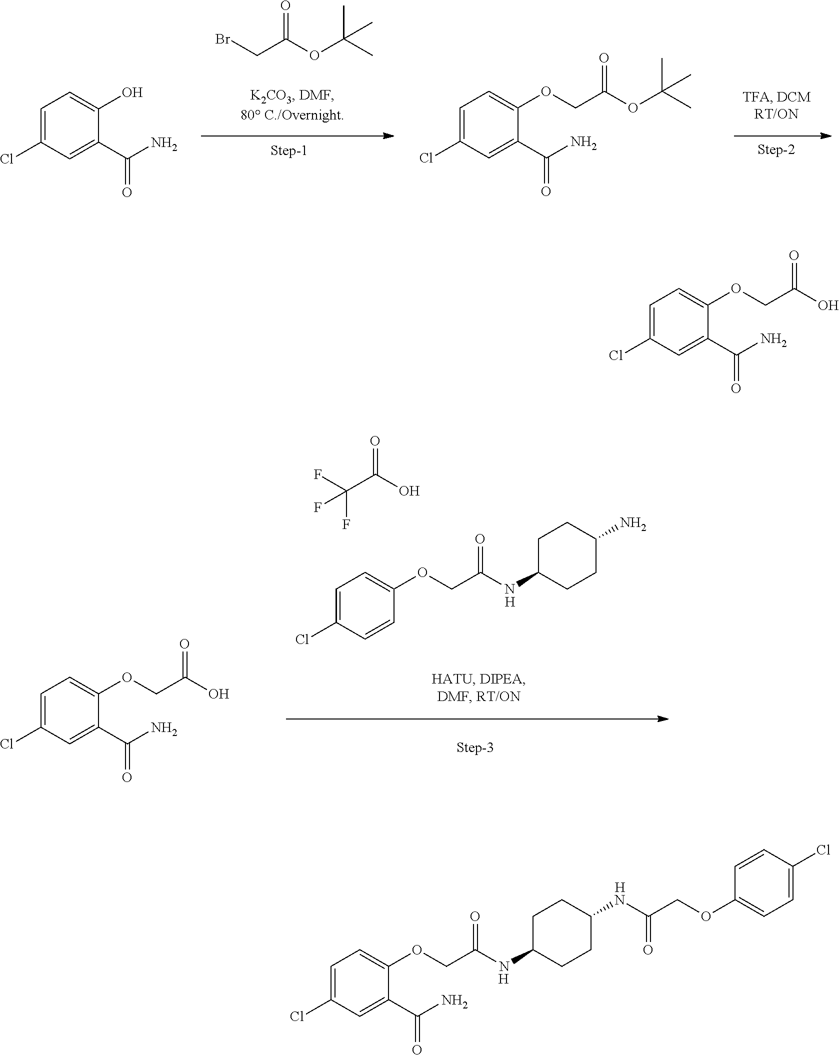

### Step 1—Synthesis of tert-butyl 2-(2-carbamoyl-4-chlorophenoxy)acetate

To a solution of 5-chloro-2-hydroxybenzamide (0.250 g, 1.461 mmol, 1.0 equiv) in DMF (05 mL) was added tert-butyl 2-bromoacetate (0.284 g, 1.461 mmol, 1.0 equiv), K₂CO₃ (0.403 g, 2.923 mmol, 2.0 equiv). The resulting reaction mixture was heated at 120° C. for overnight. Product formation was confirmed by ¹H NMR. After completion of reaction, the mixture was diluted with water (50 mL×2) The resulting solid was filtered off, washed with water (20 mL×4) and dried under vacuum to obtain tert-butyl 2-(2-carbamoyl-4-chlorophenoxy)acetate (0.240 g, 57% Yield) as a gray solid. ¹H NMR (400 MHz, DMSO-d₆) δ 8.03 (br. s., 1H) 7.85 (d, J=2.63 Hz, 2H) 7.53 (dd, J=8.77, 3.07 Hz, 1H) 7.14 (d, J=8.77 Hz, 1H) 4.85 (s, 2H) 1.45 (s, 9H).

### Step 2—Synthesis of 2-(2-carbamoyl-4-chlorophenoxy)acetic acid

To a stirred solution of tert-butyl 2-(2-carbamoyl-4-chlorophenoxy)acetate (0.100 g, 0.349 mmol, 1.0 equiv) in DCM (4 mL), was added TFA (0.4 mL) and the resultant reaction mixture was stirred at RT for 4 h under nitrogen atmosphere. Reaction was monitored by TLC and LCMS. After completion of reaction, the reaction mixture was concentrated under reduced pressure. Crude compound was crystallized in hexane (10 mL) and diethyl ether, filtered off and dried under vacuum to obtain to obtain 2-(2-carbamoyl-4-chlorophenoxy)acetic acid (0.080 g, 99% Yield) as a brown solid. ¹H NMR (400 MHz, DMSO-d₆) δ 13.38 (br. s., 1H) 8.09 (br. s., 1H) 7.67-7.93 (m, 2H) 7.53 (dd, J=8.77, 2.63 Hz, 1H) 7.15 (d, J=8.77 Hz, 1H) 4.87 (s, 2H).

### Step 3: Synthesis of trans-5-chloro-2-(2-(4-(2-(4-chlorophenoxy)acetamido)cyclohexyl)amino)-2-oxoethoxy)benzamide

To a stirred solution of 2-(2-carbamoyl-4-chlorophenoxy)acetic acid (0.100 g, 0.252 mmol, 1.0 equiv) in DMF (03 mL) was added HATU (0.192 g, 0.505 mmol, 2.0 equiv) at RT and stirred for 10 minutes. Then N-(4-aminocyclohexyl)-2-(4-chlorophenoxy)acetamide 2,2,2-trifluoroacetate (0.058 g, 0.252 mmol, 1.0 equiv) was added followed by the addition of DIPEA (0.13 mL, 0.757 mmol, 3.0 equiv). The resulting reaction mixture was allowed to stir at RT for overnight. Product formation was confirmed by LCMS. The reaction mixture was diluted with water (20 mL×4). The resulting solid was filtered off, washed with water (20 mL) and dried under vacuum to obtain trans-5-chloro-2-(2-(4-(2-(4-chlorophenoxy)acetamido)cyclohexyl)amino)-2-oxoethoxy)benzamide (Compound 92—0.010 g, 10% Yield) as a brown solid. LCMS 493.1 [M+H]⁺; ¹H NMR (400 MHz, DMSO-d₆) δ 8.30 (d, J=7.89 Hz, 1H) 8.23 (br. s., 1H) 7.96 (d, J=8.33 Hz, 1H) 7.76 (br. s., 1H) 7.69 (d, J=2.63 Hz, 1H) 7.34 (m, J=9.21 Hz, 2H) 7.09 (d, J=8.77 Hz, 1H) 6.96 (m, J=8.77 Hz, 2H) 4.65 (s, 2H) 4.44 (s, 2H) 3.57 (br. s., 3H) 1.66-1.91 (m, 3H) 1.41 (br. s., 1H) 1.20-1.38 (m, 3H).

### Example 43

### Synthesis of trans-2-(4-chlorophenoxy)-N-(4-((3-(4-chlorophenyl)ureido)methyl)cyclohexyl)acetamide

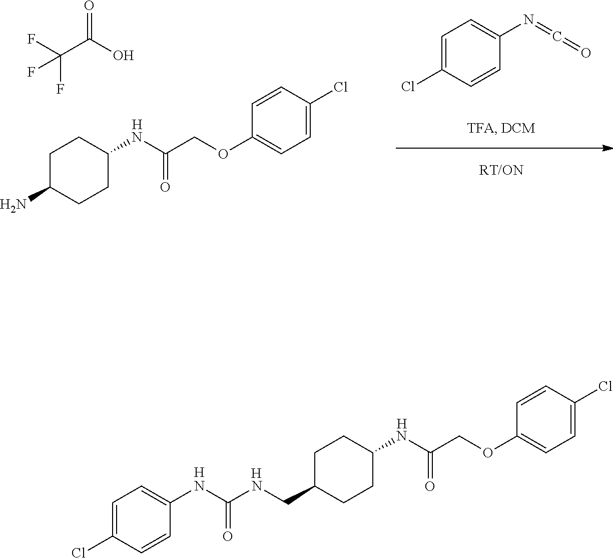

To a solution of trans-N-(4-aminocyclohexyl)-2-(4-chlorophenoxy)acetamide 2,2,2-trifluoroacetate (0.200 g, 0.50 mmol, 1.0 equiv) in DCM (1.5 mL) was added triethylamine (0.148 g, 1.47 mmol, 3.0 equiv) RT. Resultant reaction mixture was allowed to stir for 10 min. 1-chloro-4-isocyanatobenzene (0.098 g, 0.64 mmol, 1.0 equiv) was added in the above mixture. The reaction mixture was allowed to stir at RT for overnight. Progress of the reaction was monitored by LCMS. Reaction solid was filtered off, washed with minimum amount of methanol (˜5 mL) to obtain trans-2-(4-chlorophenoxy)-N-(4-((3-(4-chlorophenyl)ureido)methyl)cyclohexyl)acetamide (Compound 93—0.06 g, 26% Yield) as an off-white solid. LCMS 450.3 [M+H]⁺; ¹H NMR (400 MHz, DMSO-d₆) δ 8.50 (s, 1H) 7.91 (d, J=7.89 Hz, 1H) 7.40 (d, J=9.21 Hz, 2H) 7.27-7.36 (m, 2H) 7.25 (m, J=8.77 Hz, 2H) 6.96 (d, J=8.77 Hz, 2H) 6.22 (t, J=5.92 Hz, 1H) 4.44 (s, 2H) 3.55 (m, 1H), 2.94 (t, J=6.14 Hz, 2H) 1.61-1.89 (m, 4H), 1.35 (m, 1H), 1.23 (d, J=12.72 Hz, 2H) 0.91-1.09 (m, 2H).

### Example 44

### Synthesis of trans-7-chloro-N-(4-(2-(4-chlorophenoxy)acetamido)cyclohexyl)-3,4-dihydro-2H-benzo[b][1,4]oxazine-2-carboxamide

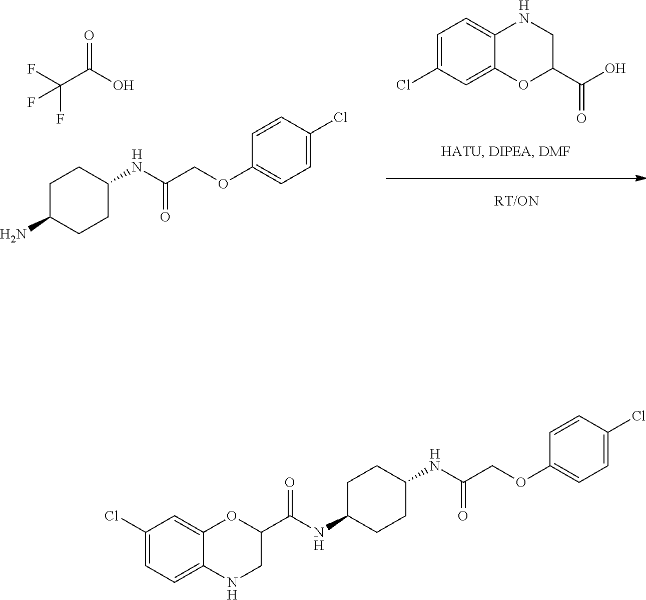

To a solution of trans-N-(-4-aminocyclohexyl)-2-(4-chlorophenoxy)acetamide 2,2,2-trifluoroacetate (0.200 g, 0.50 mmol, 1.0 equiv) in DMF (1.5 mL) was added 7-chloro-3,4-dihydro-2H-benzo[b][1,4]oxazine-2-carboxylic acid (0.107 g, 0.50 mmol, 1.0 equiv) and HATU (0.380 g, 1.00 mmol, 2.0 equiv) followed by the addition of DIPEA (0.129 mL, 1.00 mmol, 2.0 equiv). The resultant reaction mixture was allowed to stir at RT for overnight. Progress of the reaction was monitored by LCMS. The reaction mixture was diluted with cold water (50 mL) and extracted with ethyl acetate (80 mL×2). Combined organic layer was washed with cold water (50 mL×4), NaHCO₃(50 mL×2), brine (50 mL×2), dried over anhydrous Na₂SO₄ and concentrated under reduced pressure. The crude product was enriched by flash chromatography (0-5% MeOH in DCM as an eluent) which was further purified by reversed phase HPLC to obtain trans-7-chloro-N-(4-(2-(4-chlorophenoxy)acetamido)cyclohexyl)-3,4-dihydro-2H-benzo[b] [1,4]oxazine-2-carboxamide (Compound 94—0.05 g, 20% Yield) as an off-white solid. LCMS 478.3 [M+H]⁺; ¹H NMR (400 MHz, DMSO-d₆) δ 7.95 (d, J=7.89 Hz, 1H) 7.82 (d, J=7.89 Hz, 1H) 7.27-7.43 (m, 2H) 6.87-7.01 (m, 2H) 6.84 (d, J=2.19 Hz, 1H) 6.65-6.73 (m, 1H) 6.58 (d, J=8.33 Hz, 1H) 6.03 (br. s., 1H) 4.34-4.55 (m, 3H) 3.57 (br. s., 2H) 3.35-3.47 (m, 1H) 3.07-3.20 (m, 1H) 1.76 (br. s., 4H)1.20-1.42 (m, 4H).

### Example 45

### Synthesis of 5-chloro-N-((1-(2-(4-chloro-3-fluorophenoxy)acetamido)piperidin-4-yl)methyl)benzofuran-2-carboxamide

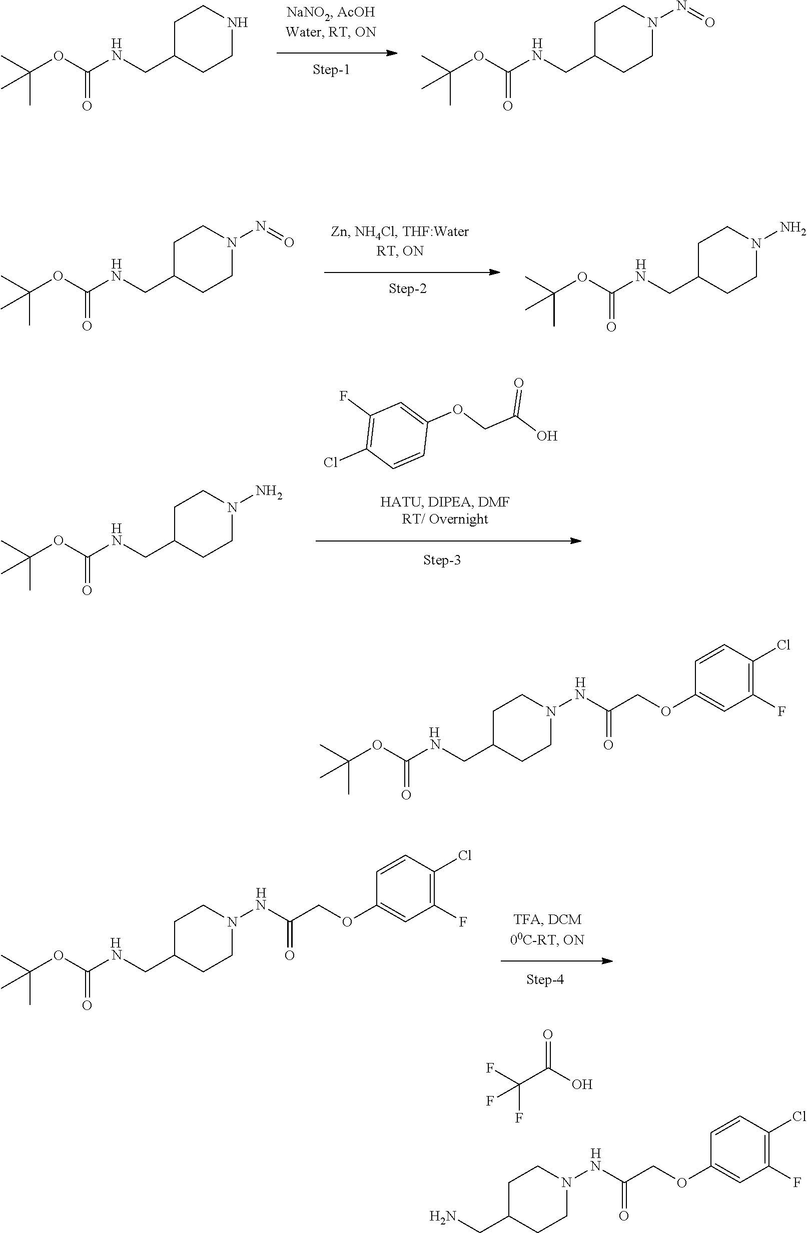

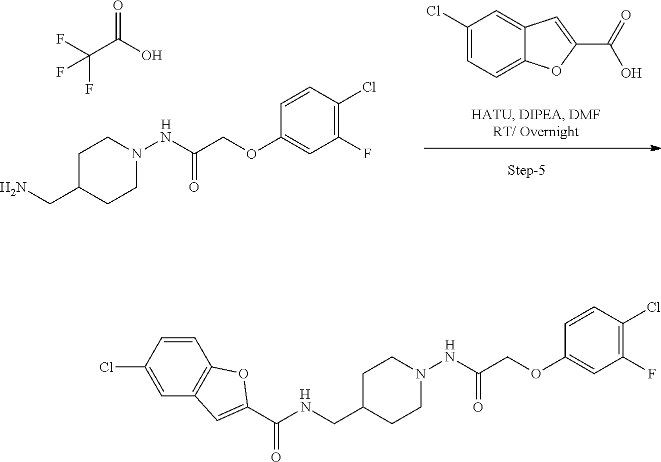

### Step 1—Synthesis of tert-butyl ((1-nitrosopiperidin-4-yl)methyl)carbamate

To a stirred solution of tert-butyl (piperidin-4-ylmethyl)carbamate (0.500 gm, 2.33 mmol, 1.0 equiv) in water (5 mL) and acetic acid (5 mL) was added a solution of sodium nitrite (0.806 gm, 1.168 mmol, 5.0 equiv) in water (2 mL) at 0° C. The reaction mixture was allowed to stir at RT for overnight. Product formation was confirmed by LCMS. The reaction mixture was diluted with water (60 mL) and extracted with EtOAc (60 mL×3). The combined organic layer was washed with sodium bicarbonate solution (50 mL×3), brine solution (50 mL), dried over anhydrous sodium sulphate and concentrated under reduced pressure to obtain tert-butyl ((1-nitrosopiperidin-4-yl)methyl)carbamate (0.300 gm, 53% Yield) as a white solid. ¹H NMR (400 MHz, CHLOROFORM-d) δ 5.04 (d, J=13.59 Hz, 1H) 4.73-4.83 (m, 1H) 4.67 (br. s., 1H) 3.66 (td, J=12.94, 3.51 Hz, 1H) 3.05 (t, J=6.36 Hz, 2H) 2.55 (td, J=13.15, 3.95 Hz, 1H) 1.98 (dt, J=13.15, 2.63 Hz, 1H) 1.79 (d, J=13.59 Hz, 1H) 1.40-1.52 (m, 9H) 1.30-1.40 (m, 1H) 1.05 (dd, J=12.28, 4.38 Hz, 1H).

### Step 2—Synthesis of tert-butyl ((1-aminopiperidin-4-yl)methyl)carbamate

To a stirred solution of tert-butyl ((1-nitrosopiperidin-4-yl)methyl)carbamate (0.300 g, 1.234 mmol, 1.0 equiv) in THF:H2O (4:4 mL) was added NH₄Cl(0.800 g, 14.81 mmol, 12.0 equiv) and then Zn dust (0.320 g, 4.938 mmol, 4.0 equiv) was added portion wise. After completion of addition the reaction mixture was stirred at RT for overnight. Progress of the reaction was monitored by TLC and LCMS. Reaction mixture was filtered through Celite® washed with DCM (60 mL). Organic layer was washed with water (40 mL×2), dried over anhydrous Na₂SO₄ and concentrated under reduced pressure to obtain tert-butyl ((1-aminopiperidin-4-yl)methyl)carbamate (0.220 g, 78% Yield) as an off white solid. ¹H NMR (400 MHz, CHLOROFORM-d) δ 4.61 (br. s., 1H) 3.11 (d, J=10.52 Hz, 2H) 3.00 (t, J=5.92 Hz, 2H) 2.61 (br. s., 1H) 1.98-2.12 (m, 2H) 1.71 (d, J=12.72 Hz, 2H) 1.43 (s, 9H) 1.29-1.38 (m, 2H) 1.22-1.29 (m, 1H).

### Step 3—Synthesis of tert-butyl ((1-(2-(4-chloro-3-fluorophenoxy)acetamido)piperidin-4-yl)methyl)carbamate

To a stirred solution of 2-(4-chloro-3-fluorophenoxy)acetic acid (0.220 g, 1.078 mmol, 1.0 equiv) in DMF (5 mL) was added HATU (0.614 g, 1.617 mmol, 1.5 equiv) at RT and stirred for 10 minutes. Then tert-butyl ((1-aminopiperidin-4-yl)methyl)carbamate (0.246 g, 1.078 mmol, 1.0 equiv) was added followed by the addition of DIPEA (0.59 mL, 3.235 mmol, 3.0 equiv). The resulting reaction mixture was allowed to stir at RT for overnight. Product formation was confirmed by LCMS. The reaction mixture was diluted with water (50 mL) and extracted with EtOAc (50 mL×2). The combined organic layer was washed with water (30 mL), brine solution (30 mL×2), dried over anhydrous sodium sulphate and concentrated under reduced pressure, to obtained tert-butyl ((1-(2-(4-chloro-3-fluorophenoxy)acetamido)piperidin-4-yl)methyl)carbamate (0.300 g, 67.11% Yield) as an off-white solid. LCMS 461.1 [M+H]⁺

### Step 4—Synthesis of N-(4-(aminomethyl)piperidin-1-yl)-2-(4-chloro-3-fluorophenoxy)acetamide 2,2,2-trifluoroacetate

To a stirred solution of tert-butyl ((1-(2-(4-chloro-3-fluorophenoxy)acetamido)piperidin-4-yl)methyl)carbamate (0.300 g, 0.722 mmol, 1.0 equiv) in DCM (5 mL), was added TFA (5 mL) and the resultant reaction mixture was stirred at RT for overnight under nitrogen atmosphere. Reaction was monitored by TLC and LCMS. After completion of reaction, the reaction mixture was concentrated under reduced pressure. The crude product was crystallized in diethyl ether to obtain N-(4-(aminomethyl)piperidin-1-yl)-2-(4-chloro-3-fluorophenoxy)acetamide 2,2,2-trifluoroacetate (0.250 g, 80% Yield) as a yellow semi solid. LCMS 316.1 [M+H]⁺

### Step 5—Synthesis 5-chloro-N-((1-(2-(4-chloro-3-fluorophenoxy)acetamido)piperidin-4-yl)methyl)benzofuran-2-carboxamide

To a stirred solution of N-(4-(aminomethyl)piperidin-1-yl)-2-(4-chloro-3-fluorophenoxy)acetamide 2,2,2-trifluoroacetate (0.250 g, 0.582 mmol, 1.0 equiv) and 5-chlorobenzofuran-2-carboxylic acid (0.137 g, 0.699 mmol, 1.2 equiv) in DMF (5 mL) was added HATU (0.332 g, 0.874 mmol, 1.5 equiv) and DIPEA (0.32 mL, 1.748 mmol, 3.0 equiv). The resulting reaction mixture was allowed to stir at RT for overnight. Product formation was confirmed by LCMS. The reaction mixture was diluted with water (40 mL) and extracted with EtOAc (40 mL×3). The combined organic layer was washed with water (30 mL), brine solution (30 mL×2), dried over anhydrous sodium sulphate and concentrated under reduced pressure to obtain 5-chloro-N-((1-(2-(4-chloro-3-fluorophenoxy)acetamido)piperidin-4-yl)methyl)benzofuran-2-carboxamide (Compound 95—0.155 g, 54% Yield) as a white solid. LCMS 494.4 [M+H]⁺; ¹H NMR (400 MHz, DMSO-d₆) δ 9.13 (s, 1H) 8.83 (t, J=5.70 Hz, 1H) 8.76 (br. s., 1H) 7.87 (s, 1H) 7.70 (d, J=8.77 Hz, 2H) 7.40-7.54 (m, 2H) 7.06 (dd, J=11.18, 2.85 Hz, 1H) 6.99 (dd, J=11.62, 2.85 Hz, 1H) 6.81-6.88 (m, 1H) 6.72-6.80 (m, 1H) 4.88 (s, 1H) 4.47 (s, 1H) 3.12-3.23 (m, 2H) 3.04 (br. s., 1H) 2.91 (d, J=10.52 Hz, 1H) 2.52-2.62 (m, 1H) 2.39 (br. s., 2H) 2.33 (br. s., 1H) 1.68 (d, J=10.09 Hz, 2H) 1.15-1.36 (m, 2H).

### Example 46

### Synthesis of 6-chloro-N-(4-((5-chlorobenzofuran-2-carboxamido)methyl)piperidin-1-yl)quinoline-2-carboxamide

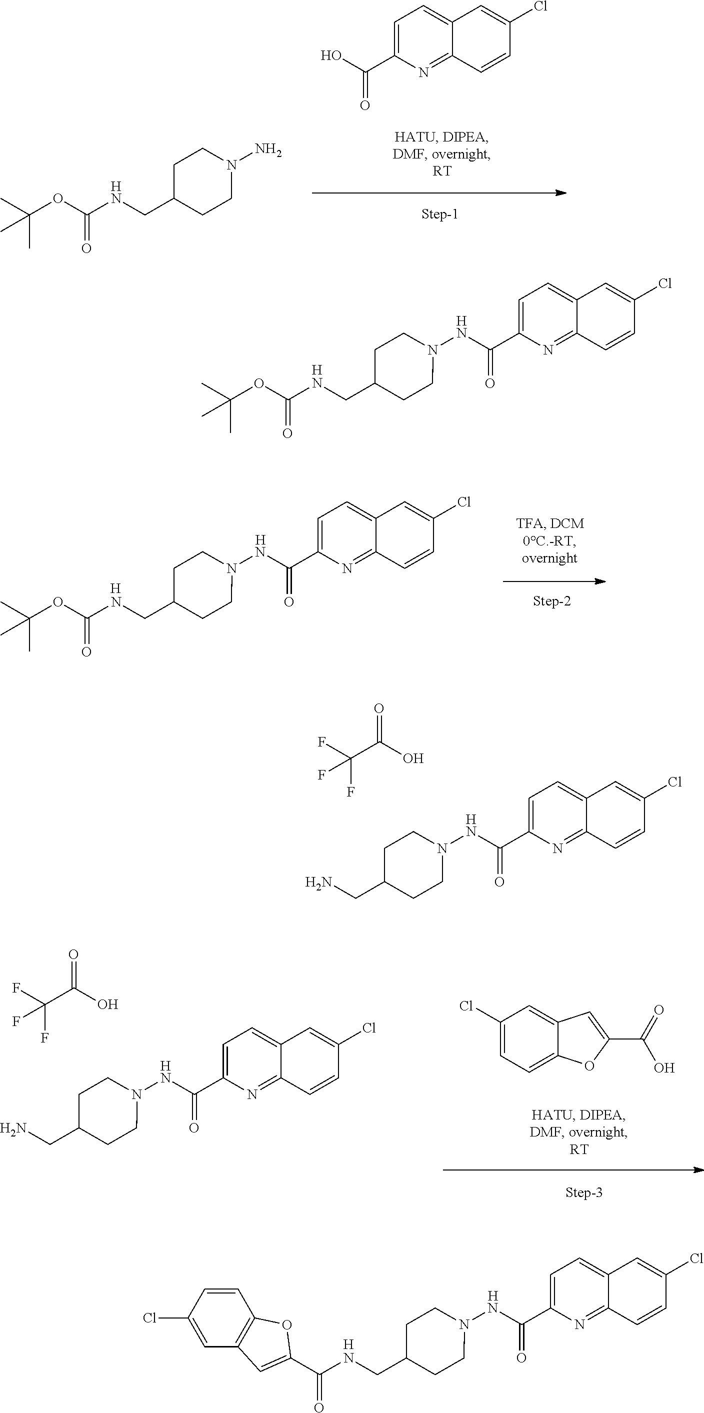

### Step 1—Synthesis of tert-butyl ((1-(6-chloroquinoline-2-carboxamido)piperidin-4-yl)methyl)carbamate

To a stirred solution of tert-butyl ((1-aminopiperidin-4-yl)methyl)carbamate (0.200 g, 0.873 mmol, 1.0 equiv) and 6-chloroquinoline-2-carboxylic acid (0.216 g, 1.048 mmol, 1.2 equiv) in DMF (5 mL) was added HATU (0.497 g, 1.31 mmol, 1.5 equiv) and DIPEA (0.48 mL, 2.62 mmol, 3.0 equiv). The resulting reaction mixture was allowed to stir at RT for overnight. Product formation was confirmed by TLC and LCMS. The reaction mixture was diluted with water (50 mL), extracted with ethyl acetate (60 mL×2), combined organic extract were washed with water (70 mL×3) and brine solution (80 mL), dried over anhydrous sodium sulphate and concentrated under reduced pressure. Crude product was purified by flash chromatography (0-5% MeOH in DCM as an eluent) to obtain tert-butyl ((1-(6-chloroquinoline-2-carboxamido)piperidin-4-yl)methyl)carbamate (0.150 g, 41% Yield) as a yellow solid. LCMS 419.5 [M+H]⁺; ¹H NMR (400 MHz, DMSO-d₆) δ 9.69 (s, 1H) 8.53 (d, J=8.33 Hz, 1H) 8.24 (s, 1H) 8.10-8.20 (m, 2H) 7.88 (d, J=8.77 Hz, 1H) 6.89 (br. s., 1H) 3.02 (d, J=9.21 Hz, 2H) 2.81-2.92 (m, 2H) 2.65-2.80 (m, 2H) 1.67 (d, J=10.96 Hz, 2H) 1.39 (s, 9H) 1.26 (t, J=5.92 Hz, 4H).

### Step 2—Synthesis of N-(4-(aminomethyl)piperidin-1-yl)-6-chloroquinoline-2-carboxamide 2,2,2-trifluoroacetate

To a stirred solution of tert-butyl ((1-(6-chloroquinoline-2-carboxamido)piperidin-4-yl)methyl)carbamate (0.150 g, 0.357 mmol, 1 equiv) in DCM (5 mL) was added TFA (0.5 mL). The resultant reaction mixture was stirred at RT for 2 hr. Progress of the reaction was monitored by TLC and NMR spectroscopy. After completion of the reaction, the reaction mixture was concentrated under reduced pressure. The crude product was crystallized with n-pentane and diethyl ether to obtain N-(4-(aminomethyl)piperidin-1-yl)-6-chloroquinoline-2-carboxamide 2,2,2-trifluoroacetate (0.100 g, 64% Yield) as an off white solid. LCMS 319.1 [M+H]⁺

### Step 3—Synthesis of 6-chloro-N-(4-((5-chlorobenzofuran-2-carboxamido)methyl)piperidin-1-yl)quinoline-2-carboxamide

To a stirred solution of N-(4-(aminomethyl)piperidin-1-yl)-6-chloroquinoline-2-carboxamide 2,2,2-trifluoroacetate (0.100 g, 0.231 mmol, 1.0 equiv) and 5-chlorobenzofuran-2-carboxylic acid (0.054 g, 0.277 mmol, 1.2 equiv) in DMF (5 mL) was added HATU (0.131 g, 0.347 mmol, 1.5 equiv) and DIPEA (0.127 mL, 0.694 mmol, 3.0 equiv). The resulting reaction mixture was allowed to stir at RT for overnight. Product formation was confirmed by TLC and LCMS. The reaction mixture was diluted with water (40 mL), extracted with ethyl acetate (50 mL×2), combined organic extract were washed with water (50 mL×3) and brine solution (50 mL), dried over anhydrous sodium sulphate and concentrated under reduced pressure. Crude product was enriched by flash chromatography (0-5% MeOH in DCM as an eluent) followed by reversed phase HPLC purification to obtain 6-chloro-N-(4-((5-chlorobenzofuran-2-carboxamido)methyl)piperidin-1-yl)quinoline-2-carboxamide (Compound 96—0.025 g, 11% Yield) as a white solid. LCMS 497.5 [M+H]⁺; ¹H NMR (400 MHz, DMSO-d₆) δ 9.71 (s, 1H) 8.85 (br. s., 1H) 8.53 (d, J=8.77 Hz, 1H) 8.24 (br. s., 1H) 8.09-8.19 (m, 2H) 7.82-7.92 (m, 2H) 7.70 (d, J=8.77 Hz, 1H) 7.41-7.57 (m, 2H) 3.22 (br. s., 2H) 3.05 (d, J=9.65 Hz, 2H) 2.77 (t, J=9.87 Hz, 2H) 1.75 (d, J=13.59 Hz, 2H) 1.65 (br. s., 1H) 1.37 (d, J=10.52 Hz, 2H).

### Example 47

### Synthesis of 5-chloro-N-(4-((5-chlorobenzofuran-2-carboxamido)methyl)piperidin-1-yl)benzofuran-2-carboxamide

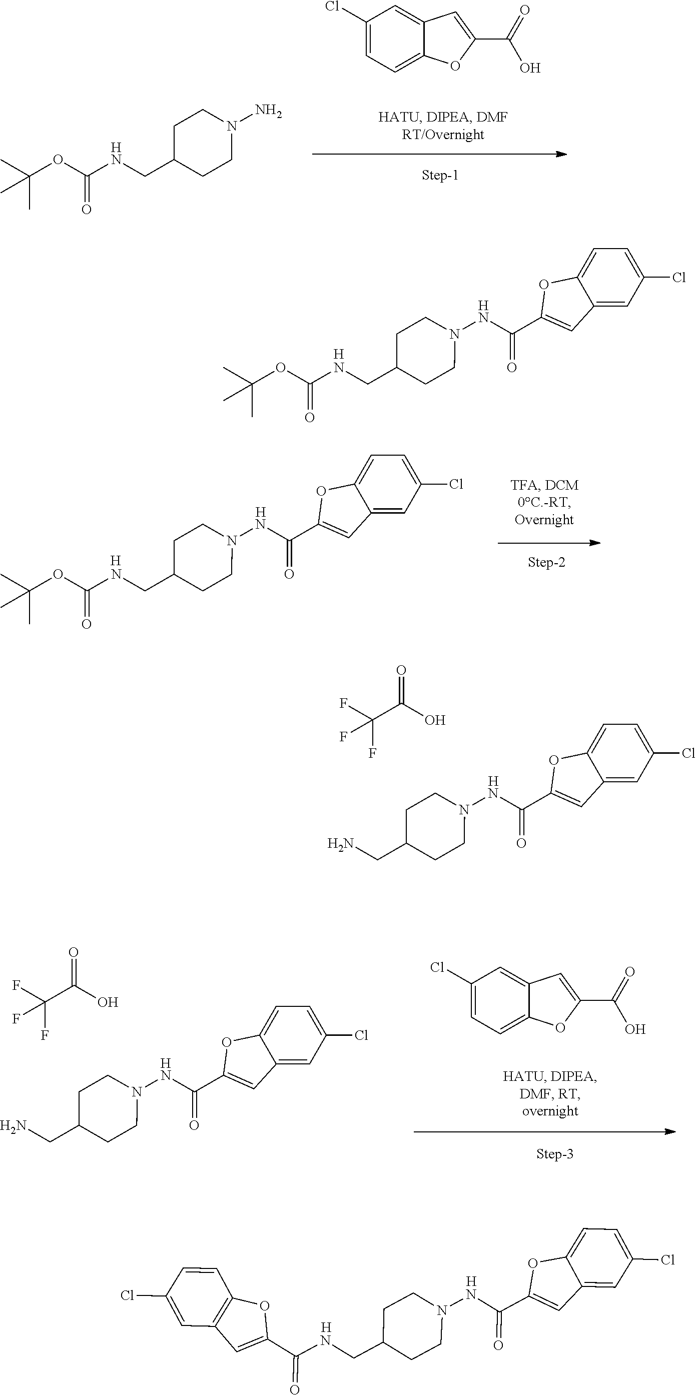

### Step 1—Synthesis of tert-butyl ((1-(5-chlorobenzofuran-2-carboxamido)piperidin-4-yl)methyl)carbamate

To a stirred solution of tert-butyl ((1-aminopiperidin-4-yl)methyl)carbamate (0.250 g, 1.091 mmol, 1.0 equiv) and 5-chlorobenzofuran-2-carboxylic acid (0.214 g, 1.091 mmol, 1.0 equiv) in DMF (5 mL) was added HATU (0.622 g, 1.637 mmol, 1.5 equiv) and DIPEA (0.6 mL, 3.275 mmol, 3.0 equiv). The resulting reaction mixture was allowed to stir at RT for overnight. Product formation was confirmed by TLC and LCMS. The reaction mixture was diluted with water (40 mL), extracted with ethyl acetate (50 mL×3), combined organic extract were washed with water (50 mL×3) and brine solution (50 mL), dried over anhydrous sodium sulphate and concentrated under reduced pressure. Crude product was purified by flash chromatography (0-5% MeOH in DCM as an eluent) to obtain tert-butyl ((1-(5-chlorobenzofuran-2-carboxamido)piperidin-4-yl)methyl)carbamate (0.370 g, 83% Yield) as a yellow solid. LCMS 408.2 [M+H]⁺; ¹H NMR (400 MHz, DMSO-d₆) δ 9.72 (s, 1H) 7.84-7.90 (m, 1H) 7.69 (d, J=8.77 Hz, 1H) 7.41-7.53 (m, 2H) 6.87 (br. s., 1H) 2.99 (d, J=10.09 Hz, 2H) 2.79-2.93 (m, 2H) 2.58-2.74 (m, 2H) 1.64 (d, J=11.84 Hz, 2H) 1.38 (s, 9H) 1.12-1.30 (m, 3H).

### Step 2—Synthesis of N-(4-(aminomethyl)piperidin-1-yl)-5-chlorobenzofuran-2-carboxamide 2,2,2-trifluoroacetate

To a stirred solution of tert-butyl ((1-(5-chlorobenzofuran-2-carboxamido)piperidin-4-yl)methyl)carbamate (0.200 g, 0.490 mmol, 1 equiv) in DCM (5 mL) was added TFA (0.5 mL). The resultant reaction mixture was stirred at RT for 2 hr. Progress of the reaction was monitored by TLC and NMR spectroscopy. After completion of the reaction, the reaction mixture was concentrated under reduced pressure. The crude product was crystallized with n-pentane and diethyl ether to obtain N-(4-(aminomethyl)piperidin-1-yl)-5-chlorobenzofuran-2-carboxamide 2,2,2-trifluoroacetate (0.110 g, 57% Yield) as an off white solid. LCMS 308.3 [M+H]⁺

### Step 3—Synthesis of 5-chloro-N-(4-((5-chlorobenzofuran-2-carboxamido)methyl)piperidin-1-yl)benzofuran-2-carboxamide

To a stirred solution of N-(4-(aminomethyl)piperidin-1-yl)-5-chlorobenzofuran-2-carboxamide 2,2,2-trifluoroacetate (0.100 g, 0.237 mmol, 1.0 equiv) and 5-chlorobenzofuran-2-carboxylic acid (0.056 g, 0.285 mmol, 1.2 equiv) in DMF (3 mL) was added HATU (0.135 g, 0.356 mmol, 1.5 equiv) and DIPEA (0.13 mL, 0.712 mmol, 3.0 equiv). The resulting reaction mixture was allowed to stir at RT for overnight. Product formation was confirmed by TLC and LCMS. The reaction mixture was diluted with water (30 mL), extracted with ethyl acetate (40 mL×3), combined organic extract were washed with water (40 mL×3) and brine solution (40 mL), dried over anhydrous sodium sulphate and concentrated under reduced pressure. Crude product was enriched by flash chromatography (0-5% MeOH in DCM as an eluent) followed by reversed phase HPLC purification to obtain 5-chloro-N-(4-((5-chlorobenzofuran-2-carboxamido)methyl)piperidin-1-yl)benzofuran-2-carboxamide (Compound 97—0.060 g, 52% Yield) as a white solid. LCMS 486.4 [M+H]⁺; ¹H NMR (400 MHz, DMSO-d₆) δ 9.76 (s, 1H) 8.84 (br. s., 1H) 7.87 (dd, J=5.48, 1.97 Hz, 2H) 7.70 (dd, J=8.55, 4.60 Hz, 2H) 7.41-7.56 (m, 4H) 3.20 (t, J=6.14 Hz, 2H) 3.02 (d, J=10.52 Hz, 2H) 2.63-2.75 (m, 2H) 1.73 (d, J=11.84 Hz, 2H) 1.62 (br. s., 1H) 1.33 (d, J=9.21 Hz, 2H).

### Example 48

### Synthesis of 5-chloro-N-((1-(2-(4-chloro-3-fluorophenoxy)acetamido)piperidin-4-yl)methyl)picolinamide

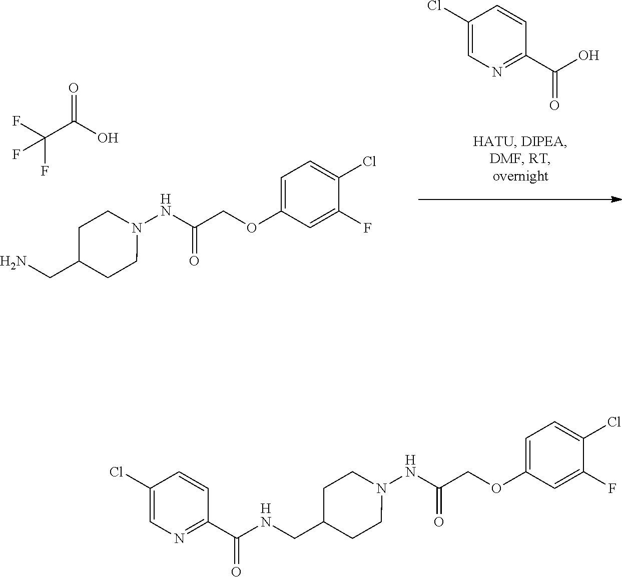

To a stirred solution of N-(4-(aminomethyl)piperidin-1-yl)-2-(4-chloro-3-fluorophenoxy)acetamide 2,2,2-trifluoroacetate (0.150 g, 0.348 mmol, 1.0 equiv) and 5-chloropicolinic acid (0.082 g, 0.523 mmol, 1.5 equiv) in DMF (5 mL) was added HATU (0.198 g, 0.523 mmol, 1.5 equiv) and DIPEA (0.19 mL, 1.04 mmol, 3.0 equiv). The resulting reaction mixture was allowed to stir at RT for overnight. Product formation was confirmed by TLC and LCMS. The reaction mixture was diluted with water (50 mL), extracted with ethyl acetate (50 mL×3), combined organic extract were washed with water (50 mL×3) and brine solution (50 mL), dried over anhydrous sodium sulphate and concentrated under reduced pressure. Crude product was enriched by flash chromatography (0-5% MeOH in DCM as an eluent) followed by reversed phase HPLC purification to obtain 5-chloro-N-((1-(2-(4-chloro-3-fluorophenoxy)acetamido)piperidin-4-yl)methyl)picolinamide (Compound 98—0.016 g, 10% Yield) as a white solid. LCMS 455.4 [M+H]⁺; ¹H NMR (400 MHz, DMSO-d₆) δ 9.28 (br. s., 1H) 8.86 (br. s., 1H) 8.70 (d, J=2.19 Hz, 1H) 8.12 (dd, J=8.33, 2.19 Hz, 1H) 8.03 (d, J=8.33 Hz, 1H) 7.39-7.54 (m, 1H) 6.95-7.08 (m, 1H) 6.75 (dd, J=8.77, 3.07 Hz, 1H) 4.87 (s, 1H) 4.48 (s, 1H) 3.19 (d, J=6.14 Hz, 2H) 3.03 (br. s., 1H) 2.94 (d, J=10.52 Hz, 1H) 2.67 (br. s., 1H) 2.55-2.64 (m, 1H) 2.33 (br. s., 1H) 1.66 (d, J=12.72 Hz, 2H) 1.17-1.36 (m, 2H).

### Example 49

### Synthesis of 2-(4-chlorophenoxy)-N-(4-((3-(4-chlorophenyl)ureido)methyl)piperidin-1-yl)acetamide

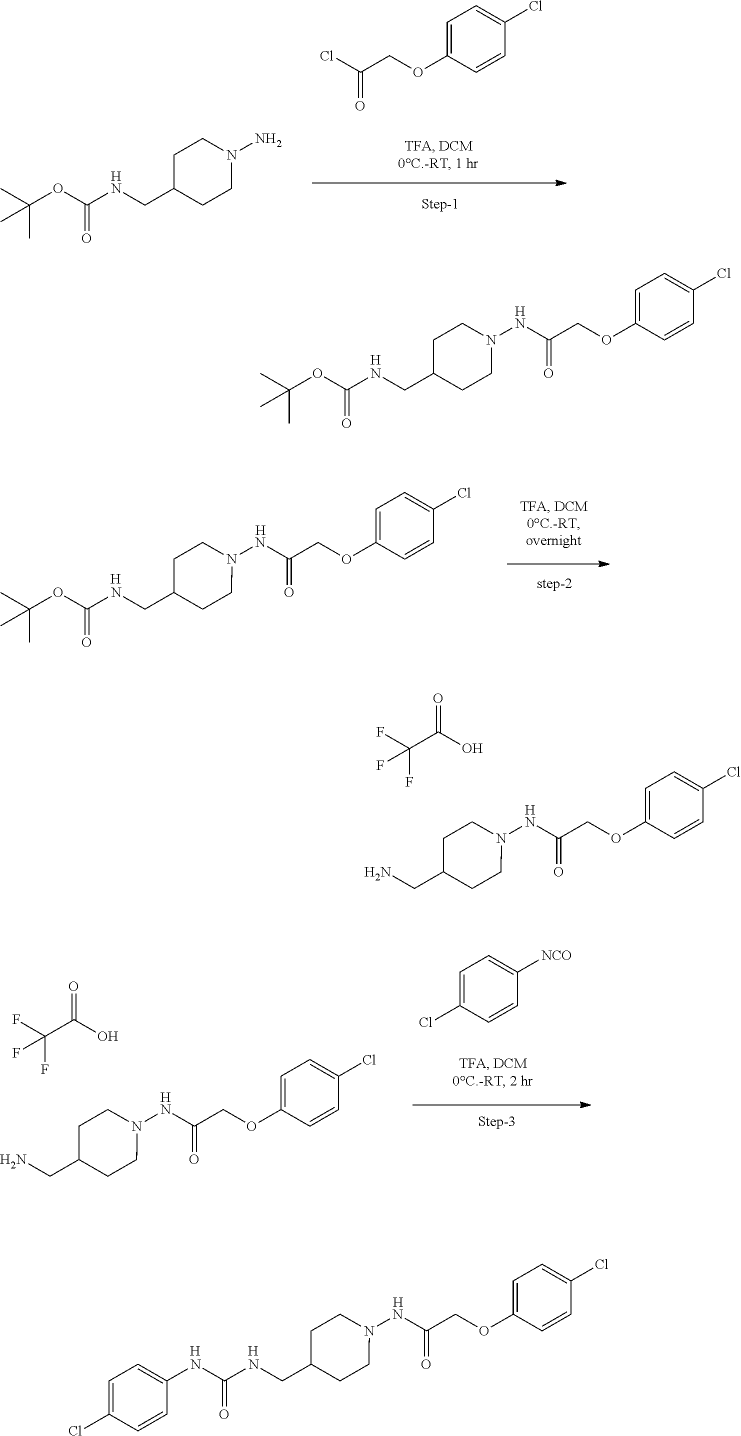

### Step 1—Synthesis of tert-butyl ((1-(2-(4-chlorophenoxy)acetamido)piperidin-4-yl)methyl)carbamate

To a stirred solution of tert-butyl ((1-aminopiperidin-4-yl)methyl)carbamate (0.200 g, 0.873 mmol, 1.0 equiv) in DCM (5 mL) was added TEA (0.37 mL, 2.62 mmol, 3.0 equiv) and 2-(4-chlorophenoxy)acetyl chloride (0.165 mL, 1.04 mmol, 1.2 equiv) at 0° C. The resulting reaction mixture was allowed to stir at RT for 1 hr. Product formation was confirmed by TLC and LCMS. The reaction mixture was diluted with water (40 mL), extracted with DCM (60 mL×3), combined organic extract were washed with water (50 mL×3) and brine solution (50 mL). Combined organic layer was dried over anhydrous sodium sulphate and concentrated. Crude product was purified by flash chromatography (0-5% MeOH in DCM as an eluent) to obtain tert-butyl ((1-(2-(4-chlorophenoxy)acetamido)piperidin-4-yl)methyl)carbamate (0.150 g, 43% Yield) as an off white solid. LCMS 398.2 [M+H]⁺; ¹H NMR (400 MHz, DMSO-d₆) δ 9.06 (s, 1H), 8.75 (b s, 1H), 7.26-7.39 (m, 2H) 6.95 (d, J=8.77 Hz, 1H) 6.87 (d, J=9.21 Hz, 1H) 4.82 (s, 1H) 4.41 (s, 1H), 3.00 (m, 1H), 2.81 (t, J=5.92 Hz, 4H), 2.39 (m, 2H), 1.61 (br. s., 2H) 1.37 (s, 9H) 1.21 (d, J=18.42 Hz, 2H)

### Step 2—Synthesis of N-(4-(aminomethyl)piperidin-1-yl)-2-(4-chlorophenoxy)acetamide 2,2,2-trifluoroacetate

To a stirred solution of tert-butyl ((1-(2-(4-chlorophenoxy)acetamido)piperidin-4-yl)methyl)carbamate (0.150 g, 0.377 mmol, 1 equiv) in DCM (4 mL) was added TFA (0.2 mL). The resultant reaction mixture was stirred at RT for overnight. Progress of the reaction was monitored by TLC and NMR spectroscopy. After completion of the reaction, the reaction mixture was concentrated under reduced pressure. The crude product was crystallized with n-pentane and diethyl ether to obtain N-(4-(aminomethyl)piperidin-1-yl)-2-(4-chlorophenoxy)acetamide 2,2,2-trifluoroacetate (0.120 g, 77.41% Yield) as a brown solid. LCMS 298.1 [M+H]⁺

### Step 3—Synthesis of 2-(4-chlorophenoxy)-N-(4-((3-(4-chlorophenyl)ureido)methyl)piperidin-1-yl)acetamide

To a stirred solution of N-(4-(aminomethyl)piperidin-1-yl)-2-(4-chlorophenoxy)acetamide 2,2,2-trifluoroacetate (0.100 g, 0.336 mmol, 1.0 equiv) in DCM (4 mL) was added TEA (0.14 mL, 1.01 mmol, 3.0 equiv) and 1-chloro-4-isocyanatobenzene (0.10 mL, 0.673 mmol, 2.0 equiv) at 0° C. The resulting reaction mixture was allowed to stir at RT for 2 hr. Product formation was confirmed by TLC and LCMS. The solid precipitate was filtered off dried under vacuum and purified by flash chromatography (0-5% MeOH in DCM as an eluent) followed by reverse phase HPLC purification to obtain 2-(4-chlorophenoxy)-N-(4-((3-(4-chlorophenyl)ureido)methyl)piperidin-1-yl)acetamide (Compound 99—0.015 g, 14% Yield) as an off white solid. LCMS 451.4 [M+H]⁺; ¹H NMR (400 MHz, DMSO-d₆) δ 9.07 (s, 1H) 8.73 (br. s., 1H) 8.55 (d, J=5.26 Hz, 1H) 7.18-7.46 (m, 4H) 6.96 (d, J=8.33 Hz, 2H) 6.88 (d, J=8.77 Hz, 1H) 6.26 (br. s., 1H) 4.83 (s, 1H) 4.42 (s, 1H) 2.98 (br. s., 2H) 2.90 (d, J=10.96 Hz, 2H) 2.33 (br. s., 2H) 1.65 (br. s., 2H) 1.39 (br. s., 1H) 1.24 (d, J=10.96 Hz, 2H).

### Example 50

### Synthesis of trans-2-(4-chlorophenoxy)-N-(4-((6-chloroquinolin-2-ylamino)methyl)cyclohexyl)acetamide

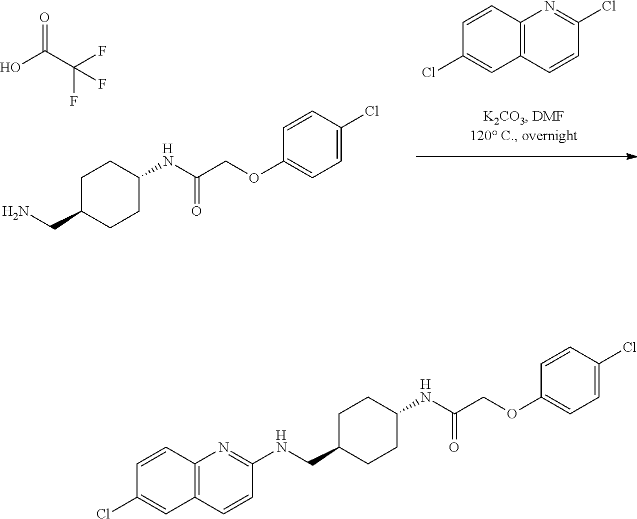

To solution of trans-N-(4-(aminomethyl)cyclohexyl)-2-(4-chlorophenoxy)acetamide 2,2,2-trifluoroacetate (0.200 g, 0.487 mmols, 1.0 equiv) in DMF (5 mL) was added K₂CO₃ (0.135 g, 0.975 mmols, 2.0 equiv) followed by the addition of 2,6-dichloroquinoline (0.097 g, 0.487 mmol, 1.0 equiv). The resulting reaction mixture was heated at 120° C. for overnight. Product formation was confirmed by TLC and LCMS. After completion of reaction the reaction mixture was diluted with water (50 mL) and extracted with ethyl acetate (100 mL×2). Combined organic layer was washed with water (20 mL×4), dried over anhydrous Na₂SO₄ and concentrated under reduced pressure. The crude product was purified by reversed phase HPLC to obtain trans-2-(4-chlorophenoxy)-N-(4-((6-chloroquinolin-2-ylamino)methyl)cyclohexyl)acetamide (Compound 100—0.050 g, 22% Yield) as an off-white solid. LCMS 558.4 [M+H]⁺; ¹H NMR (400 MHz, DMSO-d₆) δ 7.89 (d, J=7.89 Hz, 1H) 7.78 (d, J=9.21 Hz, 1H) 7.67 (d, J=1.75 Hz, 1H) 7.37-7.53 (m, 2H) 7.32 (m, J=8.77 Hz, 2H) 7.13 (t, J=5.48 Hz, 1H) 6.95 (m, J=8.77 Hz, 2H) 6.80 (d, J=9.21 Hz, 1H) 4.42 (s, 2H) 3.59 (d, J=8.33 Hz, 1H) 3.22 (t, J=5.92 Hz, 2H) 1.82 (d, J=13.15 Hz, 2H) 1.75 (br. s., 1H) 1.54 (br. s., 1H) 1.13-1.30 (m, 2H) 0.91-1.10 (m, 2H).

### Example 51

### Synthesis of trans-N-((4-(2-(4-chloro-3-fluorophenoxy)acetamido)cyclohexyl)methyl)-6-(trifluoromethyl)nicotinamide

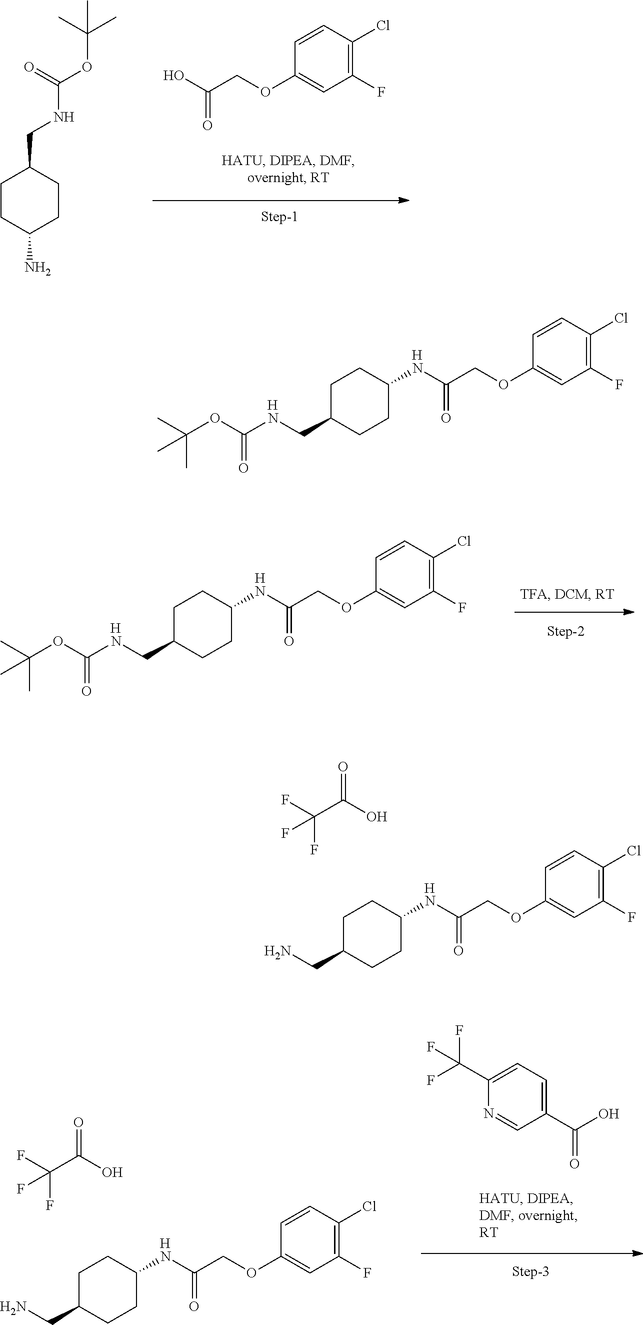

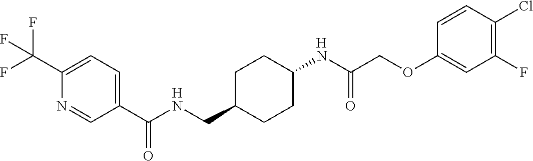

Step 1: Synthesis of trans-tert-butyl (4-(2-(4-chloro-3-fluorophenoxy)acetamido)cyclohexyl)methylcarbamate

To a stirred mixture of trans-tert-butyl (4-aminocyclohexyl)methylcarbamate (1000 mg, 4.38 mmol, 1.0 equiv), 2-(4-chloro-3-fluorophenoxy)acetic acid (1070 mg, 5.26 mmol, 1 equiv) & HATU (2500 mg, 6.57 mmol, 1.5 equiv) in DMF (10 mL) was added DIPEA (3.1 mL, 17.52 mmol, 4 equiv) and the resultant reaction mixture was stirred at RT for overnight. Reaction was monitored by LCMS. After completion of reaction, the reaction mixture was poured into ice cold water (50 ml). The resulting solid was filtered off and dried under vacuum to obtain trans-tert-butyl (4-(2-(4-chloro-3-fluorophenoxy)acetamido)cyclohexyl)methylcarbamate (1700 mg, 93.8%) as a white solid. LCMS 415.1 [M+H]⁺; ¹H NMR (400 MHz, DMSO-d₆) δ 7.92 (d, J=8.3 Hz, 1H), 7.49 (t, J=8.8 Hz, 1H), 7.06 (dd, J=2.6, 11.4 Hz, 1H), 6.91-6.70 (m, 2H), 4.48 (s, 2H), 3.54 (d, J=8.3 Hz, 1H), 2.76 (t, J=6.1 Hz, 2H), 1.83-1.54 (m, 4H), 1.37 (s, 9H), 1.22-1.03 (m, 2H), 1.00-0.75 (m, 2H).

### Step 2: Synthesis of trans-N-(4-(aminomethyl)cyclohexyl)-2-(4-chloro-3-fluorophenoxy)acetamide 2,2,2-trifluoroacetate

To a stirred solution of trans-tert-butyl (4-(2-(4-chloro-3-fluorophenoxy)acetamido)cyclohexyl)methylcarbamate (1700 mg, 4.11 mmol, 1 equiv) in DCM (30 mL), was added TFA (1.7 mL) and the resultant reaction mixture was stirred at RT for overnight under nitrogen atmosphere. Reaction was monitored by LCMS. After completion of reaction, the reaction mixture was concentrated under reduced pressure to obtain crude compound which was crystallized in diethyl ether to obtain trans-N-(4-(aminomethyl)cyclohexyl)-2-(4-chloro-3-fluorophenoxy)acetamide 2,2,2-trifluoroacetate (1600 mg, 91.1%) as an off white solid. LCMS 315.2 [M+H]⁺; ¹H NMR (400 MHz, DMSO-d₆) δ 7.96 (d, J=7.9 Hz, 1H), 7.70 (br. s., 2H), 7.50 (t, J=8.8 Hz, 1H), 7.06 (dd, J=2.6, 11.4 Hz, 1H), 6.85 (dd, J=2.0, 9.0 Hz, 1H), 4.49 (s, 2H), 3.66-3.48 (m, 1H), 2.67 (t, J=5.9 Hz, 2H), 1.85-1.60 (m, 4H), 1.48 (br. s., 1H), 1.35-1.12 (m, 2H), 1.10-0.88 (m, 2H).

### Step 3: Synthesis of trans-N-((4-(2-(4-chloro-3-fluorophenoxy)acetamido)cyclohexyl)methyl)-6-(trifluoromethyl)nicotinamide

To a stirred mixture of trans-N-(4-(aminomethyl)cyclohexyl)-2-(4-chloro-3-fluorophenoxy)acetamide 2,2,2-trifluoroacetate (50 mg, 0.117 mmol, 1 equiv), 6-(trifluoromethyl)nicotinic acid (27 mg, 0.14 mmol, 1.0 equiv) & HATU (67 mg, 0.175 mmol, 1.5 equiv) in DMF (1 mL) was added DIPEA (0.09 mL, 0.467 mmol, 4.0 equiv) and the resultant reaction mixture was stirred at RT for overnight. Reaction was monitored by LCMS. After completion of reaction, the reaction mixture was poured into ice cold water (10 ml). The resulting solid was filtered off and dried under vacuum to obtain crude product which was crystallized in diethyl ether to obtain trans-N-((4-(2-(4-chloro-3-fluorophenoxy)acetamido)cyclohexyl)methyl)-6-(trifluoromethyl)nicotinamide (Compound 101—55 mg, 96.83%) as an off white solid. LCMS 488.5 [M+H]⁺; ¹H NMR (400 MHz, DMSO-d₆) δ 9.13 (s, 1H), 8.85 (br. s., 1H), 8.45 (d, J=7.9 Hz, 1H), 8.04 (d, J=7.9 Hz, 1H), 7.94 (d, J=8.3 Hz, 1H), 7.49 (t, J=9.0 Hz, 1H), 7.06 (d, J=11.0 Hz, 1H), 6.84 (d, J=6.6 Hz, 1H), 4.49 (s, 2H), 3.58 (br. s., 1H), 3.16 (t, J=6.1 Hz, 2H), 1.78 (d, J=9.6 Hz, 4H), 1.52 (br. s., 1H), 1.24 (d, J=10.1 Hz, 2H), 1.10-0.96 (m, 2H).

### Example 52

### Synthesis of trans-N-((4-(2-(4-chloro-3-fluorophenoxy)acetamido)cyclohexyl)methyl)-6-methoxypicolinamide

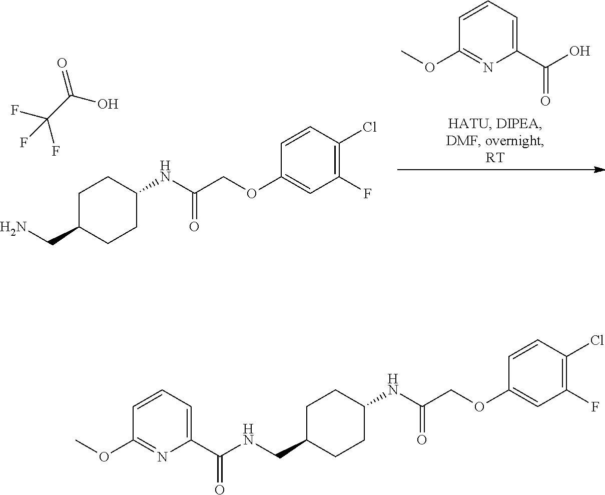 [SMILES: O=C(O)C(F)(F)F ; NC[C@H]1CC[C@H](NC(=O)COc2ccc(Cl)c(F)c2)CC1 ; COc1cccc(C(=O)O)n1 ; COc1cccc(C(=O)NC[C@H]2CC[C@H](NC(=O)COc3ccc(Cl)c(F)c3)CC2)n1]

To a stirred mixture of trans-N-(4-(aminomethyl)cyclohexyl)-2-(4-chloro-3-fluorophenoxy)acetamide 2,2,2-trifluoroacetate (100 mg, 0.234 mmol, 1 equiv), 6-methoxypicolinic acid (43 mg, 0.280 mmol, 1 equiv) & HATU (133 mg, 0.350 mmol, 1.5 equiv) in DMF (2 mL) was added DIPEA (0.17 mL, 0.934 mmol, 4 equiv) and the resultant reaction mixture was stirred at RT for overnight. Reaction was monitored by LCMS. After completion of reaction, the reaction mixture was poured into ice cold water (10 ml). The resulting solid was filtered off and dried under vacuum to obtain crude product which was crystallized in methanol to obtain trans-N-((4-(2-(4-chloro-3-fluorophenoxy)acetamido)cyclohexyl)methyl)-6-methoxypicolinamide (Compound 102—50 mg, 35.5%) as an off white solid. LCMS 450.4 [M+H]⁺; ¹H NMR (400 MHz, DMSO-d₆) δ 8.59-8.48 (m, 1H), 7.94 (d, J=7.9 Hz, 1H), 7.86 (t, J=7.9 Hz, 1H), 7.61 (d, J=7.0 Hz, 1H), 7.49 (t, J=9.0 Hz, 1H), 7.06 (dd, J=2.6, 11.4 Hz, 1H), 7.01 (d, J=8.3 Hz, 1H), 6.85 (d, J=9.2 Hz, 1H), 4.49 (s, 2H), 3.98 (s, 3H), 3.59 (br. s., 1H), 3.17 (t, J=6.4 Hz, 2H), 1.76 (t, J=13.2 Hz, 4H), 1.54 (br. s., 1H), 1.36-1.14 (m, 2H), 1.11-0.99 (m, 2H).

### Example 53

### Synthesis of trans-N-((4-(2-(4-chloro-3-fluorophenoxy)acetamido)cyclohexyl)methyl)-6-methylpicolinamide

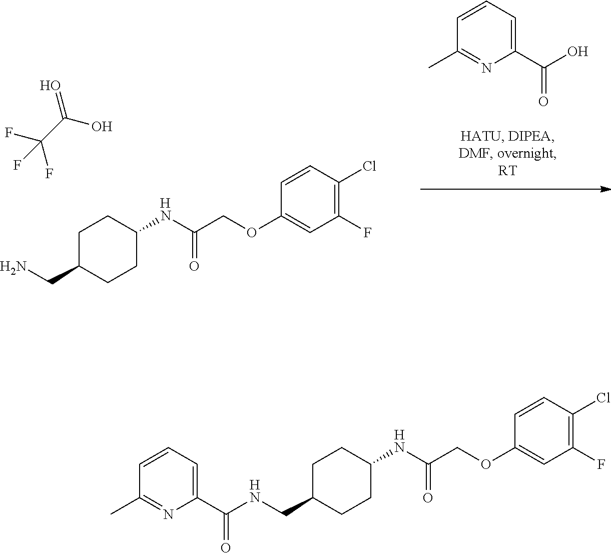

To a stirred mixture of trans-N-(4-(aminomethyl)cyclohexyl)-2-(4-chloro-3-fluorophenoxy)acetamide 2,2,2-trifluoroacetate (100 mg, 0.234 mmol, 1 equiv), 6-methylpicolinic acid (38 mg, 0.280 mmol, 1 equiv) & HATU (133 mg, 0.350 mmol, 1.5 equiv) in DMF (1 mL) was added DIPEA (0.17 mL, 0.934 mmol, 4 equiv) and the resultant reaction mixture was stirred at RT for overnight. Reaction was monitored by LCMS. After completion of reaction, the reaction mixture was poured into ice cold water (10 ml). The resulting solid was filtered of and dried under vacuum. The crude product was purified by flash chromatography (0-2% methanol in DCM as an eluent) to obtain trans-N-((4-(2-(4-chloro-3-fluorophenoxy)acetamido)cyclohexyl)methyl)-6-methylpicolinamide (Compound 103—15 mg, 14.9%) as an off white solid. LCMS 434.4 [M+H]⁺; ¹H NMR (400 MHz, DMSO-d₆) δ 8.45 (s, 1H), 8.49 (s, 1H), 7.95 (d, J=7.9 Hz, 1H), 7.67 (t, J=7.9 Hz, 1H), 7.49 (t, J=8.8 Hz, 1H), 7.20 (d, J=6.6 Hz, 1H), 7.06 (dd, J=2.6, 11.4 Hz, 1H), 6.84 (d, J=6.1 Hz, 1H), 6.70 (d, J=8.3 Hz, 1H), 4.48 (s, 2H), 3.57 (br. s., 1H), 3.12 (t, J=6.4 Hz, 2H), 1.75 (t, J=12.7 Hz, 4H), 1.48 (br. s., 1H), 1.31-1.13 (m, 2H), 1.09-0.90 (m, 2H).

### Example 54

### Synthesis of trans-N-((4-(2-(4-chloro-3-fluorophenoxy)acetamido)cyclohexyl)methyl)-6-hydroxypicolinamide

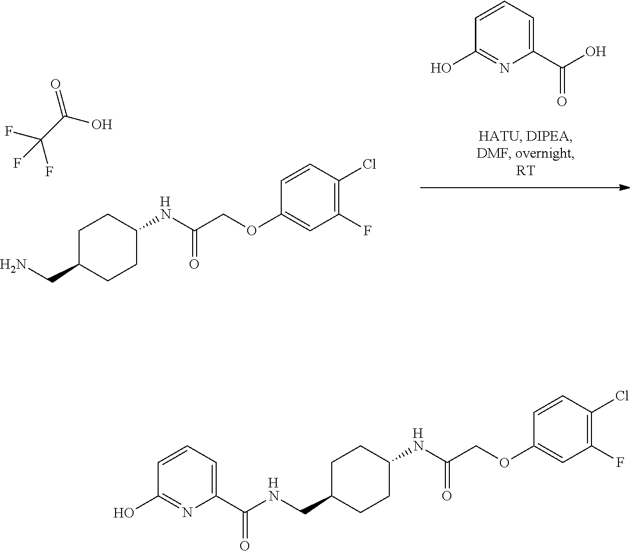

To a stirred mixture of trans-N-(4-(aminomethyl)cyclohexyl)-2-(4-chloro-3-fluorophenoxy)acetamide 2,2,2-trifluoroacetate (100 mg, 0.234 mmol, 1.0 equiv), 6-hydroxypicolinic acid (32.4 mg, 0.234 mmol, 1.0 equiv) & HATU (133 mg, 0.350 mmol, 1.5 equiv) in DMF (3 mL) was added DIPEA (0.17 mL, 0.934 mmol, 4 equiv) and the resultant reaction mixture was stirred at RT for overnight. Reaction was monitored by LCMS. After completion of reaction, the reaction mixture was poured into ice cold water (10 ml). The resulting solid was Filtered off and dried under vacuum. The crude product was purified by reversed phase HPLC to obtain trans-N-((4-(2-(4-chloro-3-fluorophenoxy)acetamido)cyclohexyl)methyl)-6-hydroxypicolinamide (Compound 104—13 mg, 12.8%) as a white solid. LCMS 436.4 [M+H]⁺; ¹H NMR (400 MHz, DMSO-d₆) δ 8.61 (t, J=6.1 Hz, 1H), 7.94 (d, J=8.3 Hz, 1H), 7.89-7.74 (m, 2H), 7.58-7.35 (m, 2H), 7.06 (dd, J=2.6, 11.4 Hz, 1H), 6.84 (td, J=1.4, 9.1 Hz, 1H), 4.48 (s, 2H), 3.62-3.48 (m, 1H), 3.16 (t, J=6.8 Hz, 2H), 2.55 (s, 3H), 1.83-1.63 (m, 4H), 1.53 (br. s., 1H), 1.28-1.14 (m, 2H), 1.06-0.94 (m, 2H).

### Example 55

### Synthesis of trans-N-((4-(2-(4-chloro-3-fluorophenoxy)acetamido)cyclohexyl)methyl)-2-hydroxyisonicotinamide

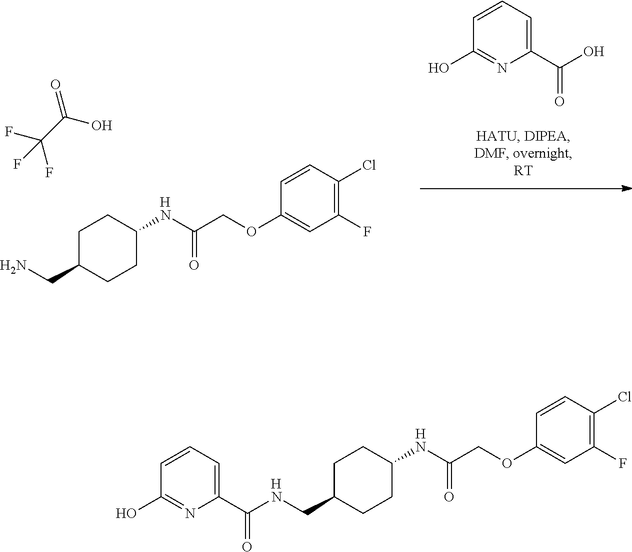

To a stirred mixture of trans-N-(4-(aminomethyl)cyclohexyl)-2-(4-chloro-3-fluorophenoxy)acetamide 2,2,2-trifluoroacetate (100 mg, 0.234 mmol, 1.0 equiv), 2-hydroxyisonicotinic acid (32.4 mg, 0.234 mmol, 1.0 equiv) & HATU (133 mg, 0.350 mmol, 1.5 equiv) in DMF (3 mL) was added DIPEA (0.17 mL, 0.934 mmol, 4 equiv) and the resultant reaction mixture was stirred at RT for overnight. Reaction was monitored by LCMS. After completion of reaction, the reaction mixture was poured into ice cold water (10 ml). The resulting solid was Filtered off and dried under vacuum. The crude product was purified by reversed phase HPLC to obtain trans-N-((4-(2-(4-chloro-3-fluorophenoxy)acetamido)cyclohexyl)methyl)-2-hydroxyisonicotinamide (Compound 105—18 mg, 17.7%) as a white solid. LCMS 436.4 [M+H]⁺; ¹H NMR (400 MHz, DMSO-d₆) δ 9.88 (br. s., 1H), 8.49 (br. s., 1H), 8.31 (dd, J=2.0, 7.2 Hz, 1H), 7.96 (d, J=8.3 Hz, 1H), 7.70 (dd, J=2.2, 6.1 Hz, 1H), 7.49 (t, J=8.8 Hz, 1H), 7.06 (dd, J=2.6, 11.4 Hz, 1H), 6.88-6.73 (m, 1H), 6.53-6.31 (m, 1H), 4.48 (s, 2H), 3.56 (br. s., 1H), 3.17 (t, J=6.1 Hz, 2H), 1.75 (t, J=14.3 Hz, 4H), 1.43 (br. s., 1H), 1.32-1.12 (m, 2H), 1.07-0.93 (m, 2H).

### Example 56

### Synthesis of trans-N-((4-(2-(4-chloro-3-fluorophenoxy)acetamido)cyclohexyl)methyl)-6-hydroxynicotinamide

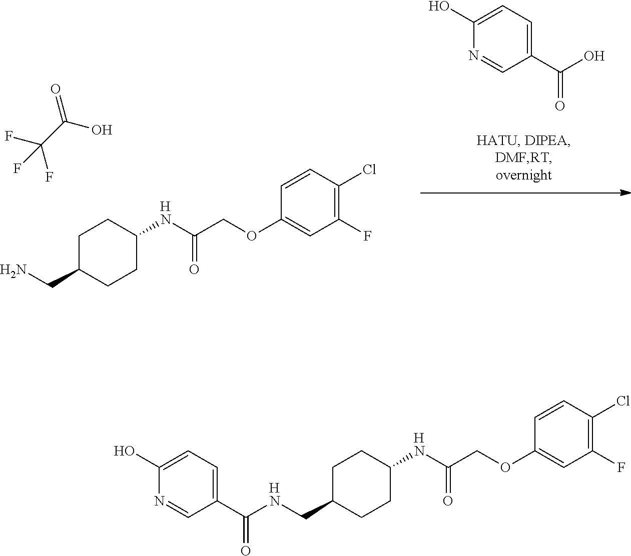

To a stirred mixture of trans-N-(4-(aminomethyl)cyclohexyl)-2-(4-chloro-3-fluorophenoxy)acetamide 2,2,2-trifluoroacetate (50 mg, 0.117 mmol, 1.0 equiv), 6-hydroxynicotinic acid (20 mg, 0.14 mmol, 1.0 equiv) & HATU (67 mg, 0.175 mmol, 1.5 equiv) in DMF (1 mL) was added DIPEA (0.09 mL, 0.467 mmol, 4 equiv) and the resultant reaction mixture was stirred at RT for overnight. Reaction was monitored by LCMS. After completion of reaction, the reaction mixture was poured into ice cold water (10 ml). The resulting solid was Filtered off and dried under vacuum. The crude product was crystallized in methanol to obtain trans-N-((4-(2-(4-chloro-3-fluorophenoxy)acetamido)cyclohexyl)methyl)-6-hydroxynicotinamide (Compound 106—15 mg, 29.5%) as an off white solid. LCMS 436.4 [M+H]⁺; ¹H NMR (400 MHz, DMSO-d₆) δ 11.93 (br. s., 1H), 8.19 (br. s., 1H), 8.02-7.87 (m, 1H), 7.86 (d, J=9.6 Hz, 2H), 7.49 (t, J=9.0 Hz, 1H), 7.06 (dd, J=3.1, 11.4 Hz, 1H), 6.84 (d, J=9.2 Hz, 1H), 6.33 (d, J=9.2 Hz, 1H), 4.48 (s, 2H), 3.58 (br. s., 1H), 3.04 (t, J=6.1 Hz, 2H), 1.86-1.63 (m, 4H), 1.44 (br. s., 1H), 1.32-1.10 (m, 2H), 1.06-0.82 (m, 2H).

### Example 57

### Synthesis of trans-6-chloro-N-((4-(2-(4-chloro-3-fluorophenoxy)acetamido)cyclohexyl)methyl)-3,4-dihydro-2H-benzo[b][1,4]oxazine-2-carboxamide

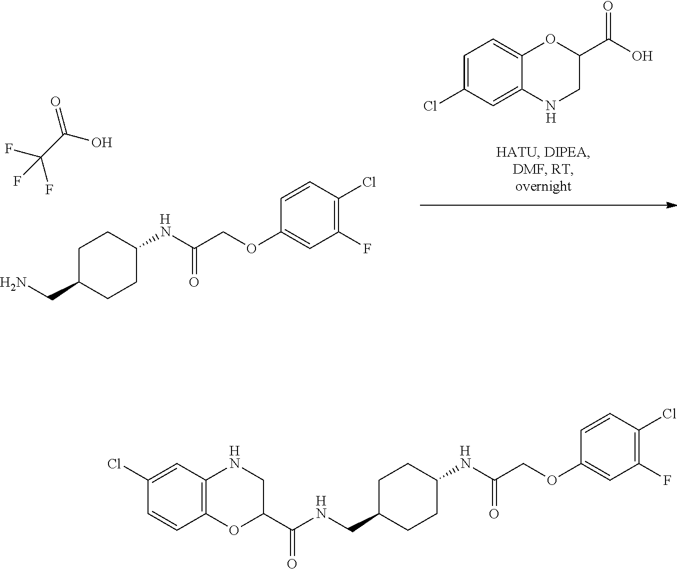

To a stirred mixture of trans-N-(4-(aminomethyl)cyclohexyl)-2-(4-chloro-3-fluorophenoxy)acetamide 2,2,2-trifluoroacetate (50 mg, 0.117 mmol, 1.0 equiv), 6-chloro-3,4-dihydro-2H-benzo[b][1,4]oxazine-2-carboxylic acid (31.3 mg, 0.175 mmol, 1.5 equiv) & HATU (67 mg, 0.175 mmol, 1.5 equiv) in DMF (2 mL) was added DIPEA (0.09 mL, 0.467 mmol, 4 equiv) and the resultant reaction mixture was stirred at RT for overnight. Reaction was monitored by LCMS. After completion of reaction, the reaction mixture was poured into ice cold water (10 ml). The resulting solid was Filtered off and dried under vacuum. The crude product was crystallized in mixture of diethyl ether and pentane to obtain trans-6-chloro-N-((4-(2-(4-chloro-3-fluorophenoxy)acetamido)cyclohexyl)methyl)-3,4-dihydro-2H-benzo[b] [1,4]oxazine-2-carboxamide (Compound 107—43 mg, 72.3%) as an off white solid. LCMS 510.4 [M+H]⁺; ¹H NMR (400 MHz, DMSO-d₆) δ 7.92 (br. s., 2H), 7.49 (t, J=8.8 Hz, 1H), 7.13-7.00 (m, 1H), 6.84 (d, J=8.3 Hz, 1H), 6.78 (d, J=8.3 Hz, 1H), 6.59 (br. s., 1H), 6.50 (d, J=8.3 Hz, 1H), 6.18 (br. s., 1H), 4.52 (br. s., 1H), 4.48 (s, 2H), 3.52 (br. s., 1H), 3.41 (br. s., 2H), 2.99 (br. s., 1H), 2.92 (br. s., 1H), 1.71 (br. s., 2H), 1.59 (br. s., 2H), 1.35 (br. s., 1H), 1.17 (d, J=11.8 Hz, 2H), 0.89 (d, J=11.4 Hz, 2H).

### Example 58

### Synthesis of trans-N-((4-(2-(4-chloro-3-fluorophenoxy)acetamido)cyclohexyl)methyl)-2-methylisonicotinamide

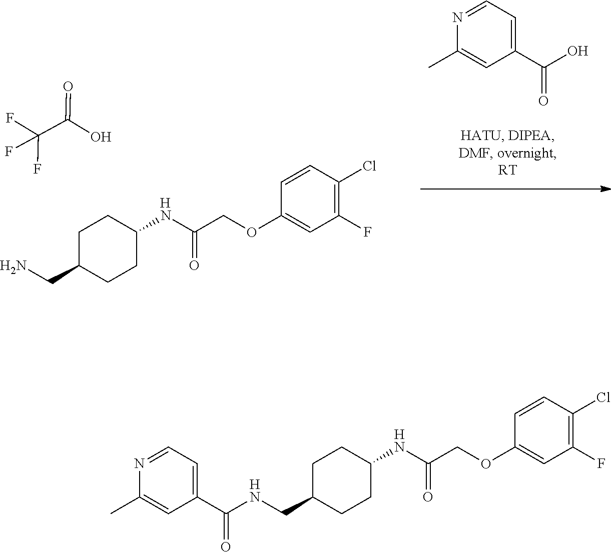

To a stirred mixture of trans-N-(4-(aminomethyl)cyclohexyl)-2-(4-chloro-3-fluorophenoxy)acetamide 2,2,2-trifluoroacetate (100 mg, 0.234 mmol, 1.0 equiv), 2-methylisonicotinic acid (64 mg, 0.467 mmol, 2.0 equiv) & HATU (133 mg, 0.350 mmol, 1.5 equiv) in DMF (1 mL) was added DIPEA (0.17 mL, 0.934 mmol, 4 equiv) and the resultant reaction mixture was stirred at RT for overnight. Reaction was monitored by LCMS. After completion of reaction, the reaction mixture was poured into ice cold water (10 ml). The resulting solid was filtered off and dried under vacuum. The crude product was washed with pentane and dried under vacuum to obtain trans-N-((4-(2-(4-chloro-3-fluorophenoxy)acetamido)cyclohexyl)methyl)-2-methylisonicotinamide (Compound 108—40 mg, 39.5%) as a white solid. LCMS 434.4 [M+H]⁺; ¹H NMR (400 MHz, DMSO-d₆) δ 8.67 (br. s., 1H), 8.56 (d, J=4.8 Hz, 1H), 7.94 (d, J=7.9 Hz, 1H), 7.61 (s, 1H), 7.56-7.35 (m, 2H), 7.06 (d, J=8.8 Hz, 1H), 6.84 (d, J=7.9 Hz, 1H), 4.48 (s, 2H), 3.57 (br. s., 1H), 3.11 (t, J=6.1 Hz, 2H), 2.52 (br. s., 3H), 1.77 (br. s., 4H), 1.50 (br. s., 1H), 1.33-1.09 (m, 2H), 1.04-0.81 (m, 2H).

### Example 59

### Synthesis of trans-N-((4-(2-(4-chloro-3-fluorophenoxy)acetamido)cyclohexyl)methyl)-6-methylnicotinamide

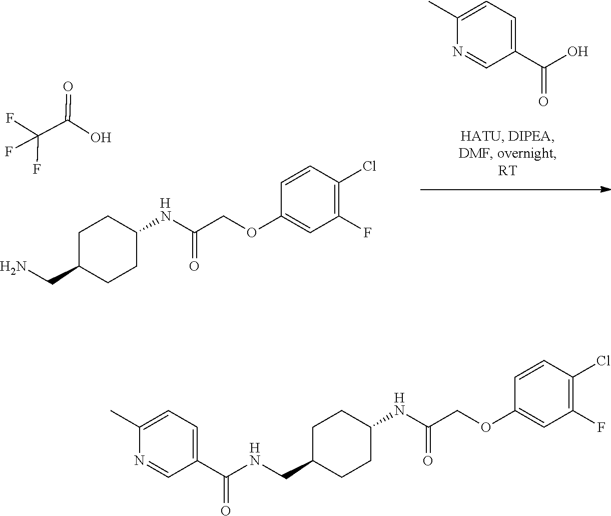

To a stirred mixture of trans-N-(4-(aminomethyl)cyclohexyl)-2-(4-chloro-3-fluorophenoxy)acetamide 2,2,2-trifluoroacetate (50 mg, 0.117 mmol, 1.0 equiv), 6-methylnicotinic acid (32 mg, 0.234 mmol, 2 equiv) & HATU (67 mg, 0.175 mmol, 1.5 equiv) in DMF (1 mL) was added DIPEA (0.09 mL, 0.464 mmol, 4 equiv) and the resultant reaction mixture was stirred at RT for overnight. Reaction was monitored by LCMS. After completion of reaction, the reaction mixture was poured into ice cold water (10 ml). The resulting solid was filtered off and dried under vacuum. The crude product was washed with diethyl ether and dried under vacuum to obtain trans-N-((4-(2-(4-chloro-3-fluorophenoxy)acetamido)cyclohexyl)methyl)-6-methylnicotinamide (Compound 109—40 mg, 39.5%) as white solid. LCMS 434.4 [M+H]⁺; ¹H NMR (400 MHz, DMSO-d₆) δ 8.87 (br. s., 1H), 8.56 (br. s., 1H), 8.07 (d, J=7.9 Hz, 1H), 7.94 (d, J=8.3 Hz, 1H), 7.49 (t, J=9.0 Hz, 1H), 7.34 (d, J=7.9 Hz, 1H), 7.06 (d, J=11.8 Hz, 1H), 6.84 (d, J=8.3 Hz, 1H), 4.48 (s, 2H), 3.59 (br. s., 1H), 3.11 (br. s., 2H), 2.67 (br. s., 3H), 1.77 (d, J=11.4 Hz, 4H), 1.50 (br. s., 1H), 1.28-1.10 (m, 2H), 1.06-0.78 (m, 2H).

### Example 60

### Synthesis of trans-methyl 6-(((4-(2-(4-chloro-3-fluorophenoxy)acetamido)cyclohexyl)methyl)carbamoyl)nicotinate

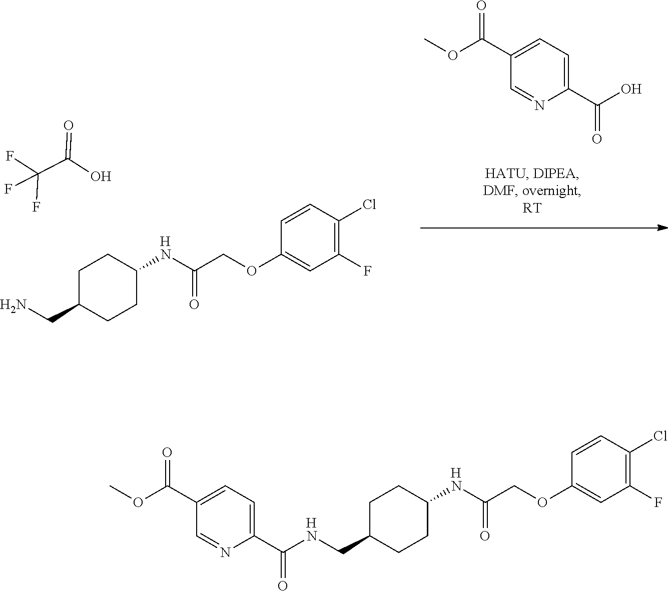

To a stirred mixture of trans-N-(4-(aminomethyl)cyclohexyl)-2-(4-chloro-3-fluorophenoxy)acetamide 2,2,2-trifluoroacetate (50 mg, 0.117 mmol, 1.0 equiv), 5-(methoxycarbonyl)picolinic acid (32 mg, 0.175 mmol, 1.2 equiv) & HATU (67 mg, 0.175 mmol, 1.5 equiv) in DMF (2 mL) was added DIPEA (0.09 mL, 0.464 mmol, 4 equiv) and the resultant reaction mixture was stirred at RT for overnight. Reaction was monitored by LCMS. After completion of reaction, the reaction mixture was poured into ice cold water (10 ml). The resulting solid was filtered off and dried under vacuum. The crude product was purified by flash chromatography (0-5% methanol in DCM as an eluent) to obtain trans-methyl 6-(((4-(2-(4-chloro-3-fluorophenoxy)acetamido)cyclohexyl)methyl)carbamoyl)nicotinate (Compound 110—10 mg, 18%) as an off white solid. LCMS 478.4 [M+H]⁺; ¹H NMR (400 MHz, DMSO-d₆) δ 9.08 (br. s., 1H), 8.96 (br. s., 1H), 8.46 (d, J=8.3 Hz, 1H), 8.14 (d, J=7.9 Hz, 1H), 7.92 (d, J=7.9 Hz, 1H), 7.47 (t, J=9.0 Hz, 1H), 7.04 (d, J=11.0 Hz, 1H), 6.82 (d, J=8.8 Hz, 1H), 4.46 (s, 2H), 3.90 (s, 3H), 3.56 (br. s., 1H), 3.15 (br. s., 2H), 1.89-1.59 (m, 4H), 1.54 (br. s., 1H), 1.20 (d, J=11.8 Hz, 2H), 0.99 (d, J=13.2 Hz, 2H).

### Example 61

### Synthesis of trans-N-((4-(2-(4-chloro-3-fluorophenoxy)acetamido)cyclohexyl)methyl)-6-(trifluoromethyl)picolinamide

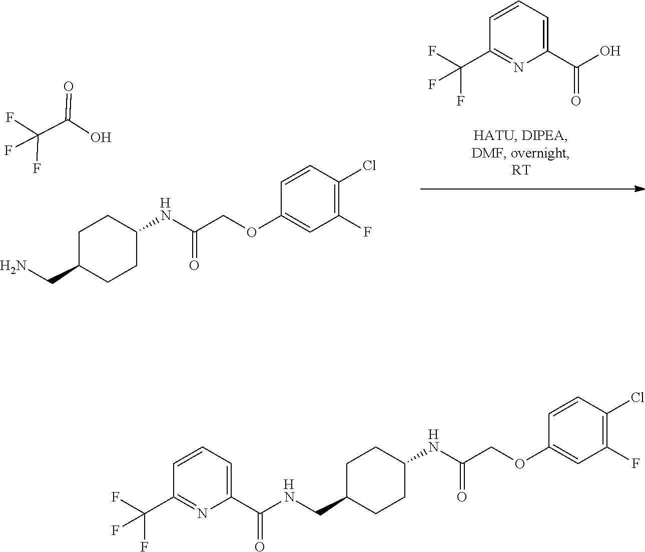

To a stirred mixture of trans-N-(4-(aminomethyl)cyclohexyl)-2-(4-chloro-3-fluorophenoxy)acetamide 2,2,2-trifluoroacetate (50 mg, 0.117 mmol, 1.0 equiv), 6-(trifluoromethyl)picolinic acid (27 mg, 0.14 mmol, 1 equiv) & HATU (67 mg, 0.175 mmol, 1.5 equiv) in DMF (1 mL) was added DIPEA (0.09 mL, 0.467 mmol, 4 equiv) and the resultant reaction mixture was stirred at RT for overnight. Reaction was monitored by LCMS. After completion of reaction, the reaction mixture was poured into ice cold water (10 ml) and extracted with ethyl acetate (2×50 mL). Organic layer washed with water (4×10 mL), brine solution (1×10 mL), dried over anhydrous Na₂SO₄ and concentrated under reduced pressure. The crude product was washed with Methanol and dried under vacuum to obtain trans-N-((4-(2-(4-chloro-3-fluorophenoxy)acetamido)cyclohexyl)methyl)-6-(trifluoromethyl)picolinamide (Compound 111—30 mg, 52.82%) as an off white solid. LCMS 488.5 [M+H]⁺; ¹H NMR (400 MHz, DMSO-d₆) δ 8.70 (t, J=5.9 Hz, 1H), 8.35-8.19 (m, 2H), 8.15-8.03 (m, 1H), 7.94 (d, J=7.9 Hz, 1H), 7.49 (t, J=8.8 Hz, 1H), 7.06 (dd, J=3.1, 11.4 Hz, 1H), 6.84 (dd, J=1.8, 8.8 Hz, 1H), 4.48 (s, 2H), 3.57 (d, J=7.9 Hz, 1H), 3.19 (t, J=6.6 Hz, 2H), 1.75 (t, J=13.8 Hz, 4H), 1.56 (br. s., 1H), 1.31-1.15 (m, 2H), 1.12-0.93 (m, 2H).

### Example 62

### Synthesis of trans-6-chloro-N-(4-((6-chloro-1,2,3,4-tetrahydronaphthalen-2-ylamino)methyl)cyclohexyl)quinoline-2-carboxamide

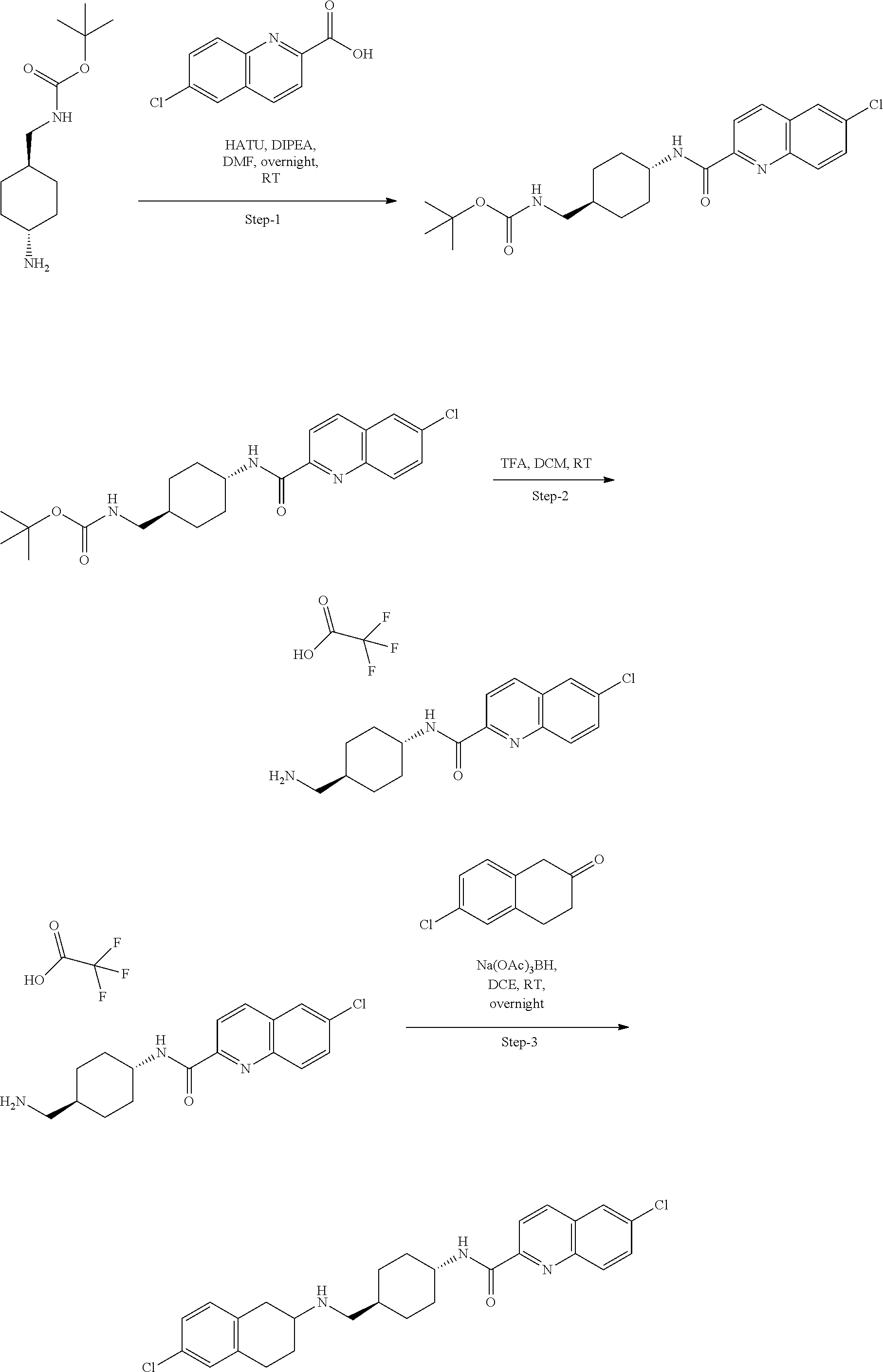

### Step-1. Synthesis of trans-tert-butyl (4-(6-chloroquinoline-2-carboxamido)cyclohexyl)methylcarbamate

To a stirred mixture of trans-tert-butyl (4-aminocyclohexyl)methylcarbamate (100 mg, 0.438 mmol, 1.0 equiv), 6-chloroquinoline-2-carboxylic acid (91 mg, 0.438 mmol, 1 equiv) & HATU (250 mg, 0.657 mmol, 1.5 equiv) in DMF (4 mL) was added DIPEA (0.35 mL, 1.75 mmol, 4.0 equiv) and the resultant reaction mixture was stirred at RT for overnight. Reaction was monitored by LCMS. After completion of reaction, the reaction mixture was poured into cold water (10 ml). The resulting solid was filtered off and dried under vacuum to obtain trans-tert-butyl (4-(6-chloroquinoline-2-carboxamido)cyclohexyl)methylcarbamate (180 mg, 98.6%) as an off white solid. LCMS 418.2 [M+H]⁺; ¹H NMR (400 MHz, DMSO-d₆) δ 8.60 (d, J=8.3 Hz, 1H), 8.54 (d, J=8.3 Hz, 1H), 8.24 (d, J=2.2 Hz, 1H), 8.22-8.09 (m, 2H), 7.88 (dd, J=2.4, 9.0 Hz, 1H), 6.86 (br. s., 1H), 3.77 (br. s., 1H), 2.80 (t, J=6.1 Hz, 2H), 2.67 (br. s., 1H), 1.88 (d, J=10.1 Hz, 2H), 1.74 (d, J=12.7 Hz, 2H), 1.45 (d, J=14.0 Hz, 2H), 1.42-1.25 (m, 9H), 0.99 (d, J=11.4 Hz, 2H).

### Step-2: Synthesis of trans-N-(4-(aminomethyl)cyclohexyl)-6-chloroquinoline-2-carboxamide 2,2,2-trifluoroacetate

To a stirred solution of trans-tert-butyl (4-(6-chloroquinoline-2-carboxamido)cyclohexyl)methylcarbamate (180 mg, 0.432 mmol, 1 equiv) in DCM (5 mL), was added TFA (0.3 mL) and the resultant reaction mixture was stirred at RT for overnight under nitrogen atmosphere. Reaction was monitored by LCMS. After completion of reaction, the reaction mixture was concentrated under reduced pressure. The crude product was crystallized in diethyl ether, dried under vacuum to obtain trans-N-(4-(aminomethyl)cyclohexyl)-6-chloroquinoline-2-carboxamide 2,2,2-trifluoroacetate (180 mg, 96.7%) as a light brown solid. LCMS 318.2 [M+H]⁺; ¹H NMR (400 MHz, DMSO-d₆) δ 8.67 (d, J=8.3 Hz, 1H), 8.54 (d, J=8.8 Hz, 1H), 8.25 (br. s., 1H), 8.17 (t, J=9.2 Hz, 2H), 7.88 (d, J=7.9 Hz, 1H), 7.75 (br. s., 2H), 3.81 (br. s., 1H), 2.71 (br. s., 2H), 1.99-1.77 (m, 4H), 1.51 (d, J=11.8 Hz, 1H), 1.23 (br. s., 2H), 1.11 (d, J=12.7 Hz, 2H).

### Step-3: Synthesis of trans-6-chloro-N-(4-((6-chloro-1,2,3,4-tetrahydronaphthalen-2-ylamino)methyl)cyclohexyl)quinoline-2-carboxamide

To a stirred mixture of trans-N-(4-(aminomethyl)cyclohexyl)-6-chloroquinoline-2-carboxamide 2,2,2-trifluoroacetate (50 mg, 0.115 mmol, 1.0 equiv), 6-chloro-3,4-dihydronaphthalen-2(1H)-one (25 mg, 0.138 mmol, 1.2 equiv) & HATU (49 mg, 0.23 mmol, 1.5 equiv) in dichloroethane (5 mL) was added sodium triacetoxyborohydride (49 mg, 0.23 mmol, 1.5 equiv) and the resultant reaction mixture was stirred at RT for overnight. Reaction was monitored by LCMS. After completion of reaction, the reaction mixture was concentrated under reduced pressure. The crude product was purified by reversed phase HPLC to obtain trans-6-chloro-N-(4-((6-chloro-1,2,3,4-tetrahydronaphthalen-2-ylamino)methyl)cyclohexyl)quinoline-2-carboxamide (Compound 112—10 mg, 18.1%) as a white solid. LCMS 482.5 [M+H]⁺; ¹H NMR (400 MHz, DMSO-d₆) δ 8.61 (d, J=8.3 Hz, 1H), 8.54 (d, J=8.3 Hz, 2H), 8.24 (br. s., 1H), 8.21-8.12 (m, 2H), 7.88 (d, J=9.2 Hz, 1H), 7.19-7.01 (m, 3H), 3.79 (br. s., 2H), 2.93 (d, J=14.5 Hz, 2H), 2.87-2.75 (m, 2H), 2.67 (br. s., 2H), 1.88 (br. s., 4H), 1.57-1.41 (m, 2H), 1.35 (br. s., 1H), 1.23 (br. s., 2H), 1.02 (d, J=12.3 Hz, 2H).

### Example 63

### Synthesis of trans-6-chloro-N-(4-((6-chloroquinolin-2-ylamino)methyl)cyclohexyl)-3,4-dihydro-2H-benzo[b][1,4]oxazine-2-carboxamide

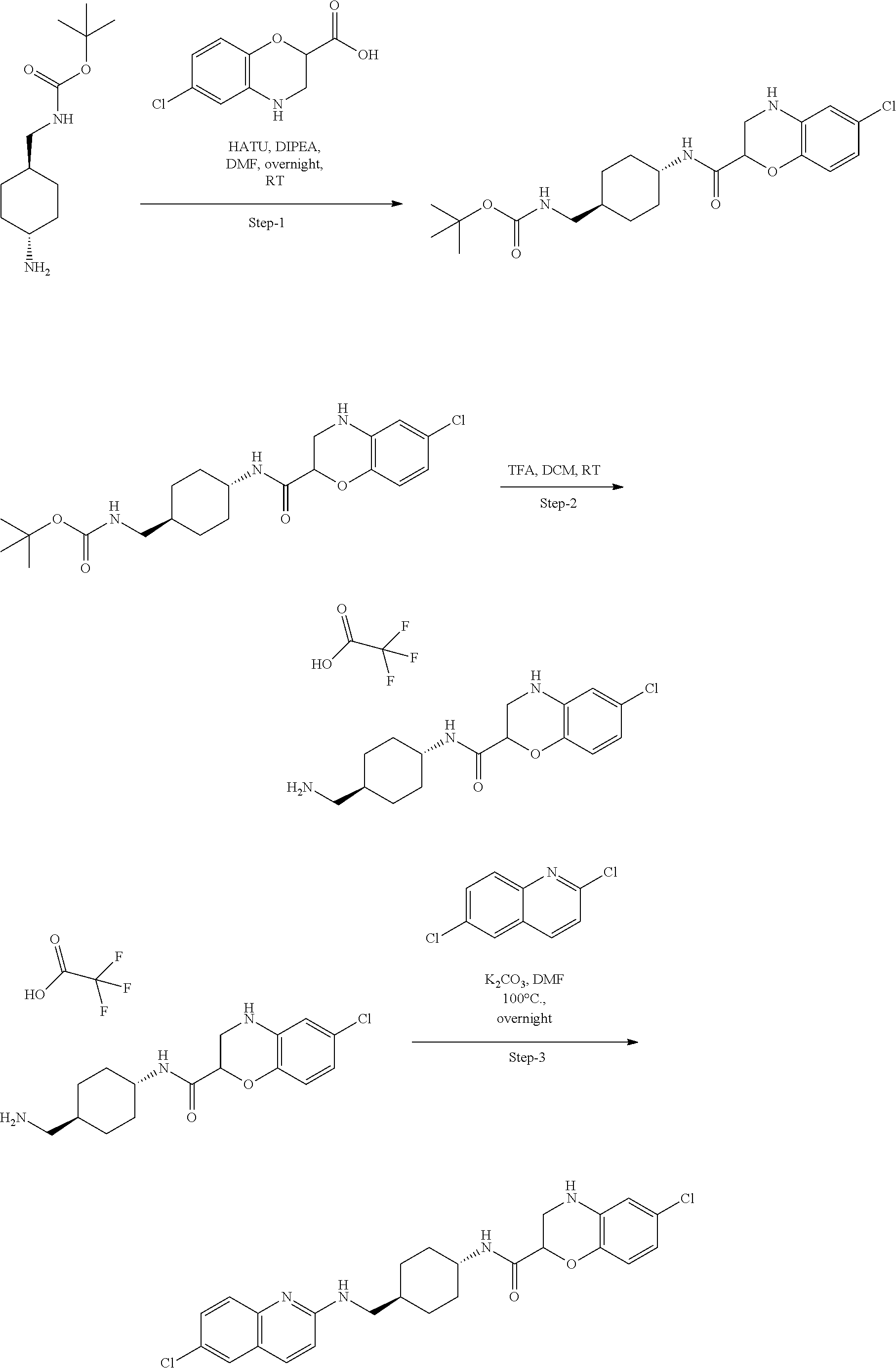

### Step-1: Synthesis of trans-tert-butyl (4-(6-chloro-3,4-dihydro-2H-benzo[b][1,4]oxazine-2-carboxamido)cyclohexyl)methylcarbamate

To a stirred mixture of trans-tert-butyl (4-aminocyclohexyl)methylcarbamate (200 mg, 0.876 mmol, 1.0 equiv) in DMF (3 mL) was added 6-chloro-3,4-dihydro-2H-benzo[b][1,4]oxazine-2-carboxylic acid (157 mg, 0.876 mmol, 1 equiv) & HATU (499 mg, 1.314 mmol, 1.5 equiv) followed by the addition of DIPEA (0.65 mL, 3.504 mmol, 4 equiv) and the resultant reaction mixture was stirred at RT for overnight. Reaction was monitored by LCMS. After completion of reaction, the reaction mixture was poured into cold water (10 ml). The resulting solid was filtered off and dried under vacuum to obtain trans-tert-butyl (4-(6-chloro-3,4-dihydro-2H-benzo[b][1,4]oxazine-2-carboxamido)cyclohexyl)methylcarbamate (360 mg, 97.16%) as an off white solid. LCMS 424.2 [M+H]⁺; ¹H NMR (400 MHz, DMSO-d₆) δ 7.75 (d, J=8.3 Hz, 1H), 6.87-6.69 (m, 2H), 6.59 (d, J=2.2 Hz, 1H), 6.50 (dd, J=2.4, 8.6 Hz, 1H), 6.18 (br. s., 1H), 4.41 (d, J=5.3 Hz, 1H), 3.51 (br. s., 1H), 3.43 (d, J=11.8 Hz, 1H), 3.20-3.07 (m, 1H), 2.76 (t, J=6.4 Hz, 2H), 1.74 (br. s., 1H), 1.66 (br. s., 3H), 1.37 (s, 9H), 1.26-1.15 (m, 2H), 0.90 (d, J=11.8 Hz, 3H) Step-2: Synthesis of trans-N-(4-(aminomethyl)cyclohexyl)-6-chloro-3,4-dihydro-2H-benzo[b][1,4]oxazine-2-carboxamide 2,2,2-trifluoroacetate

To a stirred solution of trans-tert-butyl (4-(6-chloro-3,4-dihydro-2H-benzo[b][1,4]oxazine-2-carboxamido)cyclohexyl)methylcarbamate (360 mg, 0.851 mmol, 1.0 equiv) in DCM (5 mL), was added TFA (0.8 mL) and the resultant reaction mixture was stirred at RT for overnight under nitrogen atmosphere. Reaction was monitored by LCMS. After completion of reaction, the reaction mixture was concentrated under reduced pressure. The crude product was crystallized in diethyl ether, dried under vacuum to obtain trans-N-(4-(aminomethyl)cyclohexyl)-6-chloro-3,4-dihydro-2H-benzo[b][1,4]oxazine-2-carboxamide 2,2,2-trifluoroacetate (300 mg, 80.68%) as an off white solid. LCMS 324.2 [M+H]⁺; ¹H NMR (400 MHz, DMSO-d₆) δ 7.80 (d, J=7.9 Hz, 1H), 7.68 (br. s., 2H), 6.78 (d, J=8.3 Hz, 1H), 6.60 (d, J=2.2 Hz, 1H), 6.50 (dd, J=2.4, 8.6 Hz, 1H), 6.19 (br. s., 1H), 4.57-4.36 (m, 1H), 3.56 (d, J=8.8 Hz, 2H), 3.17 (dd, J=7.7, 12.1 Hz, 2H), 2.66 (br. s., 2H), 1.76 (d, J=14.9 Hz, 4H), 1.47 (br. s., 1H), 1.32-1.15 (m, 2H), 1.01 (d, J=12.3 Hz, 2H).

### Step-3: Synthesis of trans-6-chloro-N-(4-((6-chloroquinolin-2-ylamino)methyl)cyclohexyl)-3,4-dihydro-2H-benzo[b][1,4]oxazine-2-carboxamide

To a stirred mixture of trans-N-(4-(aminomethyl)cyclohexyl)-6-chloro-3,4-dihydro-2H-benzo[b][1,4]oxazine-2-carboxamide 2,2,2-trifluoroacetate (100 mg, 0.228 mmol, 1.0 equiv), K₂CO₃ (94 mg, 0.684 mmol, 2.0) in DMF (2 mL) was added 2,6-dichloroquinoline (67 mg, 0.343 mmol, 1.5 equiv) and the resultant reaction mixture was refluxed at 100° C. for overnight. Reaction was monitored by LCMS. After completion of reaction, the mixture was diluted with water (10 mL) and extracted with ethyl acetate (10 mL×2). Combined organic extracts were washed with water (20 mL×4), dried over anhydrous Na₂SO₄ and concentrated. The crude product was purified by reverse phase HPLC to obtain trans-6-chloro-N-(4-((6-chloroquinolin-2-ylamino)methyl)cyclohexyl)-3,4-dihydro-2H-benzo[b][1,4]oxazine-2-carboxamide (Compound 113—5 mg, 4.5%) as an off white solid. LCMS 485.5 [M+H]⁺; ¹H NMR (400 MHz, DMSO-d₆) δ 7.80 (d, J=9.2 Hz, 2H), 7.69 (d, J=2.6 Hz, 1H), 7.51-7.38 (m, 2H), 7.16 (br. s., 1H), 6.77 (d, J=8.3 Hz, 1H), 6.82 (d, J=9.2 Hz, 1H), 6.59 (d, J=2.6 Hz, 1H), 6.49 (dd, J=2.6, 8.8 Hz, 1H), 6.19 (br. s., 1H), 4.43-4.33 (m, 2H), 3.57 (br. s., 2H), 3.22-3.04 (m, 2H), 1.82 (br. s., 2H), 1.74 (br. s., 1H), 1.63 (br. s., 2H), 1.30-1.17 (m, 2H), 1.05 (d, J=12.7 Hz, 2H).

### Example 64

### Synthesis of trans-6-chloro-N-(4-((6-chloroquinolin-2-ylamino)methyl)cyclohexyl)quinoline-2-carboxamide

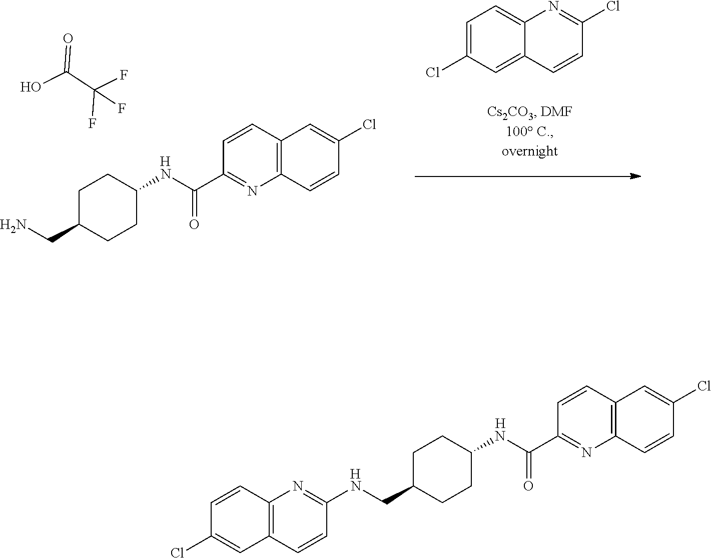

To a stirred mixture of trans-N-(4-(aminomethyl)cyclohexyl)-6-chloroquinoline-2-carboxamide 2,2,2-trifluoroacetate (50 mg, 0.115 mmol, 1.0 equiv), Cs₂CO₃ (112 mg, 0.345 mmol, 3.0) in DMF (2 mL) was added 2,6-dichloroquinoline (34 mg, 0.343 mmol, 1.5 equiv) and the resultant reaction mixture was refluxed at 100° C. for overnight. Reaction was monitored by LCMS. After completion of reaction, the mixture was diluted with water (10 mL) and extracted with ethyl acetate (10 mL×2). Combined organic extracts were washed with water (10 mL×4), dried over anhydrous Na₂SO4 and concentrated. The crude product was purified by reversed phase HPLC to obtain trans-6-chloro-N-(4-((6-chloroquinolin-2-ylamino)methyl)cyclohexyl)quinoline-2-carboxamide (Compound 114—6 mg, 10.9%) as an off white solid. LCMS 479.4 [M+H]⁺; ¹H NMR (400 MHz, DMSO-d₆) δ 8.62 (d, J=8.8 Hz, 1H), 8.54 (d, J=8.8 Hz, 1H), 8.24 (d, J=2.2 Hz, 1H), 8.19-8.06 (m, 2H), 7.87 (dd, J=2.4, 9.0 Hz, 1H), 7.81 (d, J=9.2 Hz, 1H), 7.70 (d, J=2.2 Hz, 1H), 7.52-7.36 (m, 2H), 7.19 (br. s., 1H), 6.84 (d, J=8.8 Hz, 1H), 3.83 (br. s., 1H), 3.28 (br. s., 2H), 1.91 (d, J=10.5 Hz, 4H), 1.63 (br. s., 1H), 1.51 (d, J=10.1 Hz, 2H), 1.28-1.04 (m, 2H).

### BIOLOGICAL EXAMPLES

### Example B1—ATF4 Expression Inhibition Assay

The ATF4 reporter was prepared by fusing the human full length 5′UTR of ATF4 (NCBI Accession No. BC022088.2) upstream of the firefly luciferase coding sequence lacking the initiator methionine. The fused sequence was cloned into pLenti-EFla-C-Myc-DDK-IRES-Puro cloning vector (Origen #PS100085) using standard methods. Virus production was carried out by using Lenti-X™ Packaging Single Shots Protocol (Clonetech #631276). Viral particles were used to transduce HEK293T cells (ATCC #CRL-3216, ATCC Manassas, Va.), which were subsequently selected with puromycin to generate stable cell line. Cells were maintained at 37° C. and 5% CO₂ in DMEM-F12 (Hyclone #SH30023.02) supplemented with 10% heat-inactivated fetal bovine serum (Gibco #16000-044), 2 mM L-glutamine (Gibco #25030-081), 100 U/ml penicillin, and 100 μg/ml streptomycin (Gibco #¹⁵¹⁴⁰-122).

HEK293T cells carrying the ATF4 luciferase reporter were plated on 96-well plates (Nunc) at 10,000 cells per well. Cells were treated two days after seeding with 100 nM thapsigargin (Tg) (Sigma-Aldrich #T9033) in the presence of different concentrations of selected compounds ranging from 1 nM to 10 μM. Cells without treatment or cells treated with Tg alone were used as controls. Assay plates containing cells were incubated for 3 hours at 37° C.

Luciferase reactions were performed using Luciferase Assay System (Promega #E1501) as specified by the manufacturer. Luminescence was read with an integration time of 1 s and a gain of 110 using a Cytation-5 multi-mode microplate reader (BioTek). Relative luminescence units were normalized to Tg treatment (0% inhibition) and untreated cells (100% inhibition) and the percentage of ATF4 inhibition was calculated.

The half-maximal inibitory concentration (IC₅₀) for the increasing of ATF4 protein levels is shown in Table 2. Under ISR stressed conditions (resulting from treatment with Tg), ATF4 expression is generally upregulated. Accordingly, inhibition of ATF4 expression as a result of the test compound indicates suppression of the ISR pathway.

TABLE 2

| Compound No. | ATF4 inhibition IC₅₀ (nM) |
|---|---|
| 1 | >1000 |
| 2 | <1000 |
| 3 | <1000 |
| 4 | <1000 |
| 5 | >1000 |
| 6 | <1000 |
| 7 | >1000 |
| 8 | >1000 |
| 9 | 72.39 |
| 10 | 106.2 |
| 11 | 0.82 |
| 12 | 119.9 |
| 13 | 18.98 |
| 14 | >10000 |
| 15 | 1.45 |
| 16 | 5.25 |
| 17 | 2.36 |
| 18 | 5.63 |
| 19 | 20.1 |
| 20 | 2.43 |
| 21 | 17.8 |
| 22 | 10.9 |
| 23 | 1.27 |
| 24 | >1000 |
| 25 | 1.56 |
| 26 | 0.6 |
| 27 | <1000 |
| 28 | 3.1 |
| 29 | 12.5 |
| 30 | <1000 |
| 31 | <1000 |
| 32 | >1000 |
| 33 | >1000 |
| 34 | >1000 |
| 83 | 19.3 |
| 84 | 18.3 |
| 85 | 44 |
| 86 | 12.3 |
| 87 | 90.2 |
| 88 | >1000 |
| 89 | <1000 |
| 90 | 61.5 |
| 91 | 28.7 |
| 92 | 52.2 |
| 93 | 3.88 |
| 94 | 35.8 |
| 95 | 2.9 |
| 96 | 1.05 |
| 97 | 0.59 |
| 98 | 30.4 |
| 99 | 14.5 |

### Example B2—Protein Synthesis Assay

Chinese hamster ovary (CHO) cells were maintained at 37° C. and 5% CO₂ in Dulbecco's Modified Eagle's Media (DMEM) supplemented with 10% fetal bovine serum, 2 mM L-glutamine, 100 U/ml penicillin, and 100 μg/ml streptomycin. After reaching 80% of confluence, cells were detached and seeded on 6 well plates in complete media, allowed to recover overnight and treated for 2 hours with 1 μM of the test compound (to assess protein synthesis levels in unstressed condition), or for 2 hours with 300 nM Tg in the presence of 1 μM of the test compound (to assess the recovery of protein synthesis in a stressed condition). Cells with Tg alone were used as controls.

After the 2 hours treatments, media were replaced by adding 10 μg/ml puromycin (Sigma Aldrich #P8833) in complete media for 30 min. Media were removed and cells were lysed with SDS-PAGE lysis buffer. Lysates were transferred to 1.5 ml tubes and sonicated for 3 min and total protein amount were quantified using BCA Protein Assay Kit (Pierce). Equal amount of protein (30 μg) was loaded on SDS-PAGE gels. Proteins were transferred onto 0.2 m PVDF membranes (BioRad) and probed with primary antibodies diluted in Tris-buffered saline supplemented with 0.1% Tween 20 (Merck #S6996184 505) and 3% bovine serum albumin (Rockland #BSA-50).

Puromycin (12D10) (Merck #MABE343) and j-actin (Sigma Aldrich #A5441) antibodies were used as primary antibody. A HRP-conjugated secondary antibody (Rockland) was employed to detect immune-reactive bands using enhanced chemiluminescence (ECL Western Blotting Substrate, Pierce). Quantification of protein bands was done by densitometry using ImageJ software.

Percent increase of protein synthesis in unstressed cells (without Tg treatment) in the presence of media alone or certain test compounds is shown in Table 3. The percentage levels were normalized to the media alone condition, which correspond to 100% protein synthesis. Certain compounds stimulated protein synthesis above baseline, indicating that these test compounds result in increased protein synthesis in unstressed cells.

Percent recovery of protein synthesis in stressed cells (with Tg treatment) due to the test compounds at 1 μM is also shown in Table 3. The levels were normalized to the media alone and Tg alone conditions, which correspond to 100% and 0% respectively.

TABLE 3

| | % Protein synthesis | % Recovery of |
|---|---|---|
| | relative to untreated | protein synthesis |
| Compound No. | (1 μM test compound) | (1 μM test compound) |
| 1 | 166.9 | 233.4 |
| 2 | 98.3 | 252.0 |
| 3 | 120.1 | 109.9 |
| 4 | 164 | 246.6 |
| 5 | 82.5 | 71.9 |
| 6 | 179.4 | 316.4 |
| 7 | 114.0 | 131.9 |
| 8 | 174.9 | 128.3 |
| 9 | 118.9 | 26.1 |
| 10 | 228.7 | 380.7 |
| 11 | 187.1 | 298.9 |
| 12 | 199.8 | 187.4 |
| 13 | 128.9 | 120.4 |
| 14 | 149.9 | 53.2 |
| 15 | 204.4 | 176.3 |
| 16 | 146.8 | 102.4 |
| 17 | 96.5 | 49.5 |
| 18 | 105.0 | 60.0 |
| 19 | 131.4 | 32.5 |
| 20 | 75.6 | 34.8 |
| 21 | 67.8 | 11.4 |
| 22 | 83.3 | 54.6 |
| 23 | 93.0 | 74.1 |
| 24 | 138.5 | 49.1 |
| 25 | 266.4 | 223.3 |
| 26 | 173.0 | 113.4 |
| 27 | 125.8 | 38.1 |
| 28 | 68.0 | 28.6 |
| 29 | 70.9 | 1.3 |
| 30 | 109.9 | 0.4 |
| 31 | 120.3 | 66.2 |
| 32 | 162.2 | 69.7 |
| 33 | 599.2 | 720.0 |
| 34 | 136.7 | 105.2 |
| 83 | 148.0 | 98.4 |
| 84 | 134.7 | 35.0 |
| 85 | 124.2 | 31.6 |
| 86 | 125.1 | 108.1 |
| 87 | 147.1 | 71.8 |
| 88 | 104.6 | 28.6 |
| 89 | 133.6 | 21.5 |
| 90 | 115.6 | −27.4 |
| 91 | 94.8 | −8.5 |
| 92 | 79.7 | −44.3 |
| 93 | 88.0 | 69.1 |
| 94 | 103.4 | 58.4 |
| 95 | 191.2 | 133.4 |
| 96 | 258.3 | 396.6 |
| 97 | 210.8 | 249.6 |
| 98 | −24.9 | 124.5 |
| 99 | −42.0 | 122.3 |

Data summarized in Tables 2 and 3 show that some compounds have differential activity in ATF4 inhibition and protein synthesis under ISR-inducing conditions. That is, some compounds are able to effectively inhibit ATF4 expression but do not restore protein synthesis. Other compounds effectively restore protein synthesis but do not inhibit ATF4 expression under ISR-inducing conditions. Still other compounds inhibit ATF4 expression and restore protein synthesis.

### Example B3—ATF4 Inhibition Assay Under Aβ Stimulation

Chinese hamster ovary (CHO) cells that stably express human APP751 incorporating the familial Alzheimer's disease mutation V717F are used as a source of A monomer and low-n oligomers. These cells, referred to as 7PA2 CHO cells, are cultured in 100 mm dishes with Dulbecco's modified Eagle's medium (DMEM) containing 10% fetal bovine serum, 2 mM L-glutamine, 100 U/ml penicillin, and 100 μg/ml penicillin, streptomycin and 200 μg/ml G418. Upon reaching 90-100% confluency, cells are washed with 5 mL of glutamine- and serum-free DMEM and incubated for approximately 16 h in 5 mL of the same DMEM. Conditioned media (CM) is collected.

SH-SY5Y cells are maintained at 37° C. and 5% CO₂ in RPMI 1640 media supplemented with 10% fetal bovine serum (FBS), penicillin and streptomycin. After reaching 80% of confluence, cells are detached and seeded on 6 well plates in complete media, allowed to recover 48 h and treated for 16 hours with CM from WT CHO cells or 7PA2 CHO cells in the presences of 1 μM of selected compounds.

After 16 hours treatment, culture media are removed and cells are lysed with SDS-PAGE lysis buffer. Lysates are transferred to 1.5 ml tubes and sonicated for 3 min. Total protein amount is quantified using BCA Protein Assay Kit (Pierce). Equal amount of proteins (30 μg) is loaded on SDS-PAGE gels. Proteins are transferred onto 0.2 μm PVDF membranes (BioRad) and probed with primary antibodies diluted in Tris-buffered saline supplemented with 0.1% Tween 20 and 3% bovine serum albumin.

ATF4 (11815) antibody is used as primary antibody (Cell Signaling Technologies). A 3-actin antibody is used as a control primary antibody. An HRP-conjugated secondary antibody (Rockland) is employed to detect immune-reactive bands using enhanced chemiluminescence (ECL Western Blotting Substrate, Pierce). Quantification of protein bands is done by densitometry using ImageJ.

Percent inhibition of ATF4 expression in SH-SY5Y cells after incubation with CM from the 7PA2 CHO cells as a result of the test compounds can be reported. Percentage of ATF4 inhibition is calculated as the percent reduction normalized to CM from 7PA2 CHO cells treatment (0% inhibition) and CM from WT CHO cells treatment (100% inhibition).

### Example B4—Fasting-Induced Muscle Atrophy

Wild type eight-weeks-old male Balb/c mice obtained from the vivarium Fundación Ciencia & Vida Chile (Santiago, Chile) are used. Mice are housed in independent plastic cages in a room maintained at 25° C. with a 12-h:12-h light:dark cycle.

Twenty-four hours before and during the 2 days of fasted procedures, animals receive oral administration via feeding tubes (15 gauge) of vehicle (50% Polyethylene glycol 400 (Sigma-Aldrich P3265) in distilled water or 10 mg/kg of test compound formulated in vehicle solution.

After 2 days of fasting the animals are sacrificed and muscles are removed from both hindlimbs. Mice with feed and water ad libitum are used as control.

During muscle atrophy, protein synthesis was reduced and protein degradation was increased as known in the art. For in vivo measurements of protein synthesis, puromycin (Sigma-Aldrich, P8833) is prepared at 0.04 μmol/g body weight in a volume of 200 μL of PBS, and subsequently administered into the animals via IP injection, 30 min prior to muscle collection.

Upon collection, muscles are immediately frozen in liquid nitrogen and then stored at −80° C. The frozen muscles are then homogenized with a T 10 basic ULTRA-TURRAX (IKa) in ice-cold buffer lysis (Cell Signaling 9803) and protease and phosphatase inhibitors (Roche). Lysates are sonicated for 3 min and centrifuged at 13,000 rpm for 20 minutes at 4° C. Protein concentration in supernatants is determined using BCA Protein Assay Kit (Pierce). Equal amount of proteins is loaded on SDS-PAGE gels. Proteins are transferred onto 0.2 um PVDF membranes (BioRad) and probed with primary antibodies diluted in Tris-buffered saline supplemented with 0.1% Tween 20 and 3% bovine serum albumin.

Puromycin (12D10) (Merck Millipore), MuRF-1 (Santa Cruz Biotechnology) and 3-actin (Sigma-Aldrich) antibodies are used as primary antibodies. A HRP-conjugated secondary antibody (Rockland) is employed to detect immune-reactive bands using enhanced chemiluminescence (ECL Western Blotting Substrate, Pierce). Quantification of protein bands is done by densitometry using ImageJ software.

For immunohistochemical analysis of cross-sectional area (CSA), muscles from control (Fed) and fasted animals are submerged individually in optimal cutting temperature (OCT) compound (Tissue-Tek; Sakura) at resting length, and frozen in isopentane cooled with liquid nitrogen. Cross-sections (10-μm thick) from the mid-belly of the muscles are obtained with a cryostat (Leica) and immunostained with puromycin antibody (12D10) (Merck Millipore). A HRP-polymer conjugated secondary antibody (Biocare Medical, MM620L) followed by diaminobenzidine substrate incubation (ImmPACT DAB-Vector, SK-4105) are employed to detect puromycinylated structures in CSA.

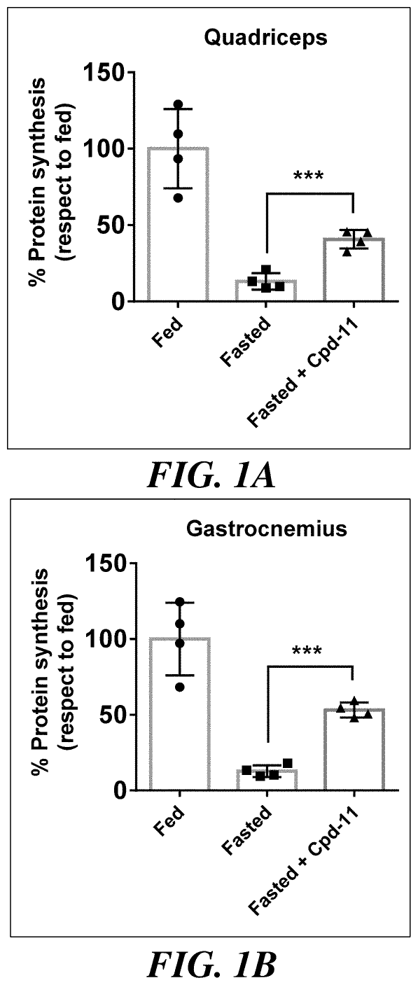

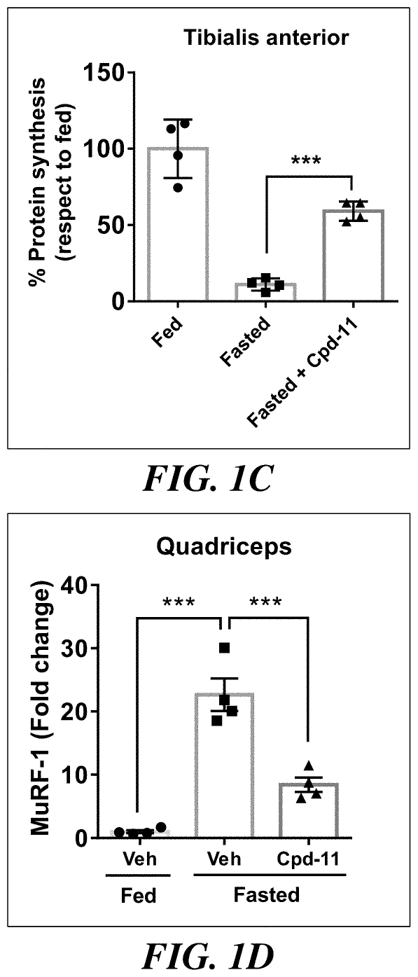

Percent of protein synthesis in quadriceps, gastrocnemius and tibialis anterior of each mouse from fed or fasted animals treated with vehicle or compound 11 is shown in FIGS. 1A, 1B and 1C respectively. The levels are normalized to 3-actin expression and percentage is calculated as the percent relative to protein synthesis levels from control mice (Fed) which correspond to 100%.

Muscle fiber CSA are visualized with a Zeiss Axio Lab.A1 microscope and an Axiocam (Zeiss) digital camera. Puromycin staining in CSA can be reported.

Expression of the muscle atrophy marker MuRF-1 in quadriceps from fed or fasted mice treated with vehicle or compound 11 is shown in FIG. 1D. The levels were normalized to β-actin expression and fold change was calculated as the level relative to MuRF-1 levels from control mice (Fed) which correspond to 1. Treatment of fasted mice with compound 11 resulted in a reduction of MuRF-1 expression compared to vehicle-treated fasted mice suggesting a reduction in muscle atrophy.

### Example B5—Cachexia-Induced Muscle Atrophy

Wild type six-weeks-old male Balb/c mice obtained from the vivarium Fundación Ciencia & Vida Chile (Santiago, Chile) are used. Mice are housed in independent plastic cages in a room maintained at 25° C. with a 12-h:12-h light:dark cycle.

1×10⁶ CT26 colon carcinoma cell line (ATCC #CRL-2638, ATCC Manassas, Va.) are injected subcutaneously in the right lower flank of each animal for induction of cachexia-induced muscle atrophy as described (Nat Commun. 2012 Jun. 12; 3:896). Non-injected animals are used as controls. At day 6 post tumor-cell injection, animals are randomized into two groups and treated with 10 mg/kg of test compound formulated in 50% Polyethylene glycol (PEG-400) in distilled water, or with vehicle (50% PEG-400 in distilled water) by daily oral gavage for 13 days.

For in vivo measurements of protein synthesis, 30 min before the study ends, animals are injected intraperitoneally with puromycin (Sigma-Aldrich, P8833) at 0.04 μmol/g body weight in a volume of 200 μL of PBS. After 13 days of daily dosage, the animals are sacrificed and gastrocnemius, quadriceps and tibialis anterior muscles are dissected and weighed from both hindlimbs to assess muscle atrophy.

Upon collection, muscles are immediately frozen in liquid nitrogen and then stored at −80° C. The frozen muscles are then homogenized with a T 10 basic ULTRA-TURRAX (IKa) in ice-cold buffer lysis (Cell Signaling 9803) and protease and phosphatase inhibitors (Roche). Lysates are sonicated for 3 min and centrifuged at 13,000 rpm for 20 minutes at 4° C. Protein concentration in supernatants is determined using BCA Protein Assay Kit (Pierce). Equal amount of protein is loaded on SDS-PAGE gels. Proteins are transferred onto 0.2 um PVDF membranes (BioRad) and probed with primary antibodies diluted in Tris-buffered saline supplemented with 0.1% Tween 20 and 3% bovine serum albumin.

Puromycin (12D10) (Merck Millipore) and β-actin (Sigma-Aldrich) antibodies are used as primary antibodies. A HRP-conjugated secondary antibody (Rockland) is employed to detect immune-reactive bands using enhanced chemiluminescence (ECL Western Blotting Substrate, Pierce). Quantification of protein bands is done by densitometry using ImageJ software.

Gastrocnemius, quadriceps and tibialis anterior muscle weight from animals injected with CT26 tumor cells and treated with either vehicle or test compound are reported.

Percent of protein synthesis in gastrocnemius, quadriceps and tibialis anterior from animals injected with CT26 tumor cells and treated with either vehicle or test compound is also reported. The levels are normalized to 3-actin expression and percentage is calculated as the percent relative to protein synthesis levels from muscle section of control mice which correspond to 100%.

### Example B6—Protein Synthesis with a Cell-Free System

The expression of the green fluorescence protein (GFP) is evaluated using the 1-Step Human In vitro Protein Expression Kit based on HeLa cell lysates (ThermoFisher Scientific). HeLa lysate, accessory proteins, reaction mix and pCFE-GFP plasmid from the kit are thawed in ice. Reactions are prepared at room temperature in a 96-well optical plate by adding 12.5 μL of HeLa lysate, 2.5 μL accessory proteins, 5 μL reaction mix, 1 g of pCFE-GFP plasmid and 1 M of test compounds in 5 μL or 5 μL of distilled H₂O as a basal expression of GFP (vehicle). A well with dH₂O instead of pCFE-GFP plasmid is used as basal autofluorescence of the reaction. All reactions are made in duplicated. Fluorescence intensity is measured by a multi-mode microplate reader (Synergy-4; Biotek) during 6-hour treatments and capturing fluorescence at 15-minute intervals with 485/20 and 528/20 excitation and emission filters.

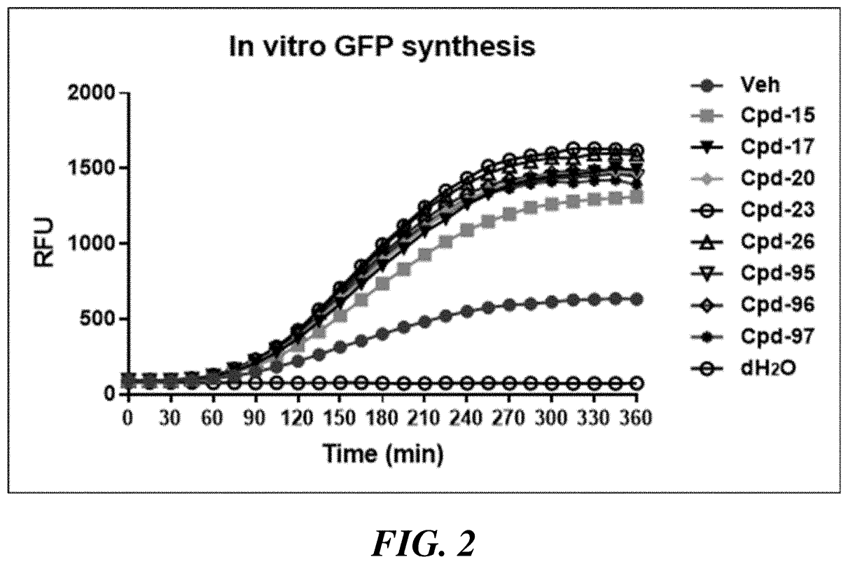

Relative fluorescence intensity (RFU) of GFP treated with either vehicle or test compounds is shown in FIG. 2. The addition of tested compounds to the kit's reaction mix increased the expression of GFP and hence its fluorescence compared to the expression obtained using the kit's reagents alone.

### Example B7—Protein Secretion in a CHO Cell-Based Assay

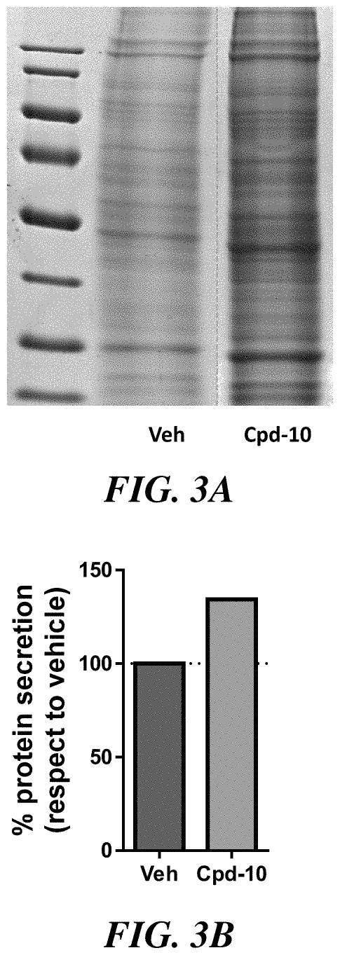

CHO cells were maintained at 37° C. and 5% CO₂ in DMEM supplemented with 10% fetal bovine serum (FBS), 2 mM L-glutamine, 100 U/ml penicillin, and 100 μg/ml streptomycin. After reaching 80% of confluence, cells were detached and seeded on 6-well plates in complete media, allowed to recover for 48 h. Cells were then washed three times with PBS and treated with compound 10 at 1 M in 1 mL of media without FBS for 24 h. Treatment with 0.1% DMSO was used as control (vehicle). After 24 h treatment, supernatant (SN) which contains secreted proteins was extracted and protease and phosphatase inhibitors (Roche) were added to each sample. SN were centrifuged at 2,000 g for 10 min to discard any cellular debris and 900 μL SN were transferred to empty microtubes with 400 μL methanol by mixing well. 200 μL of chloroform was added to the mix and then samples were centrifuged at 14,000 g for 2 minutes. Top aqueous layer was discarded by pipetting off and 400 μL methanol is added to each sample by mixing well. Samples were then centrifuged at 17,000 g for 8 minutes and methanol was discarded by pipetting off without disturbing the protein pellet. Samples were left dry at room temperature and pellets are resuspended with SDS-PAGE sample buffer. Secreted proteins were analyzed by SDS-PAGE and Coomassie staining. Gels were stained with 0.1% Coomassie Blue R250 in 10% acetic acid, 50% methanol, and 40% H₂O for 20 minutes. Stained gel was then washed twice for 2 hours with 10% acetic acid, 50% methanol and 40% dH₂O until the Coomassie Blue background is nearly clear. Photograph of the gel was taken in a gel imaging system (Chemidoc-BioRad) and it is shown in FIG. 3A. Dotted line indicates that picture of the same gel was cut in order to plot the lanes corresponding to vehicle and Cpd-10 treatment one beside the other. Percent of protein secretion in CHO cells treated with vehicle or 1 μM compound 10 (Cpd-10) is shown in FIG. 3B. Percentage was calculated as the percent relative to protein secretion levels from CHO cells treated with vehicle which correspond to 100%. Tested compound 10 increased secretion of proteins in CHO cells.

### Example B8—Secretion of Ig-Kappa by ARH Cells

ARH cells were maintained at 37° C. and 5% CO₂ and seeded at a density of 500,000 cell/well in RPMI supplemented with, 100 U/ml penicillin, and 100 μg/ml streptomycin in a 12-well culture plate. Cells were treated either with vehicle (0.1% DMSO) or 1 M of compound 10 for 24 hours. Then, culture media with cells were recovered in microtubes and centrifuged at 500 g for 5 minutes. Supernatants (SN) which contain secreted proteins were extracted and protease and phosphatase inhibitors (Roche) were added to each sample. SN were centrifuged at 2,000 g for 10 min to discard any cellular debris and 900 μL SN were transferred to empty microtubes with 400 μL methanol by mixing well. 200 μL of chloroform was added to the mix and then samples were centrifuged at 14,000 g for 2 minutes. Top aqueous layer was discarded by pipetting off and 400 μL methanol was added to each sample by mixing well. Samples were then centrifuged at 17,000 g for 8 minutes and methanol was discarded by pipetting off without disturbing the protein pellet. Samples were left dry at room temperature and pellets were resuspended with SDS-PAGE sample buffer. Secreted proteins were loaded on SDS-PAGE gels. Proteins were transferred onto 0.2 μm PVDF membranes (BioRad) and probed with anti Ig-kappa light chain (Abcam) primary antibody diluted in Tris-buffered saline supplemented with 0.1% Tween 20 and 3% bovine serum albumin. A HRP-conjugated secondary antibody (Rockland) was employed to detect immune-reactive bands using enhanced chemiluminescence (ECL Western Blotting Substrate, Pierce). Quantification of protein bands was done by densitometry using ImageJ software.

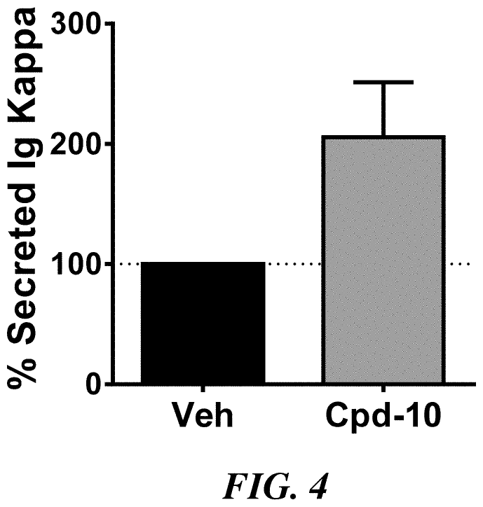

Percent of secreted Ig kappa light chain by ARH cells treated with vehicle or compound 10 from three independent experiments is shown in FIG. 4. Percentage was calculated as the percent relative to Ig kappa secretion levels from ARH cells treated with vehicle which correspond to 100%. Tested compound increased the secretion of the immunoglobulin light chain kappa in the lymphoblastic ARH cell line.

### Example B9—Secretion of Wnt-3A by L-Wnt-3A Cells

L-Wnt-3A cells (ATCC® CRL-2647™) were maintained at 37° C. and 5% CO₂ in DMEM supplemented with 10% fetal bovine serum (FBS), 2 mM L-glutamine, 100 U/ml penicillin, and 100 μg/ml streptomycin. After reaching 80% of confluence, cells were detached and seeded on 6-well plates in complete media, allowed to recover for 48 h. Cells were then washed three times with PBS and treated with vehicle (1% DMSO) or 1 M compound 10 in DMEM with reduced FBS (1%) for 24 hours. Then, culture media with cells were recovered in microtubes and centrifuged at 500 g for 5 minutes. Supernatants (SN) which contain secreted proteins were extracted and protease and phosphatase inhibitors (Roche) were added to each sample. SN were centrifuged at 2,000 g for 10 min to discard any cellular debris and 900 μL SN were transferred to empty microtubes with 400 L methanol by mixing well. 200 μL of chloroform was added to the mix and then samples were centrifuged at 14,000 g for 2 minutes. Top aqueous layer was discarded by pipetting off and 400 μL methanol was added to each sample by mixing well. Samples were then centrifuged at 17,000 g for 8 minutes and methanol was discarded by pipetting off without disturbing the protein pellet. Samples were left dry at room temperature and pellets were resuspended with SDS-PAGE sample buffer. Secreted proteins were loaded on SDS-PAGE gels. Proteins were transferred onto 0.2 μm PVDF membranes (BioRad) and probed with anti Wnt-3A primary antibody (Cell Signaling Technologies) diluted in Tris-buffered saline supplemented with 0.1% Tween 20 and 3% bovine serum albumin. A HRP-conjugated secondary antibody (Rockland) was employed to detect immune-reactive bands using enhanced chemiluminescence (ECL Western Blotting Substrate, Pierce). Quantification of protein bands was done by densitometry using ImageJ software.

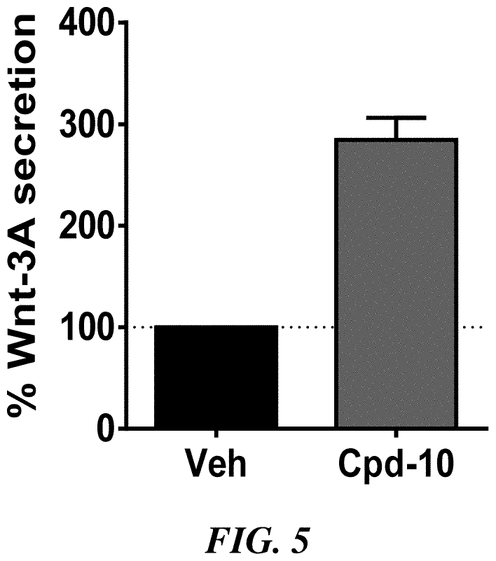

Percent of secreted Wnt-3A by L-Wnt3A cells treated with vehicle or compound 10 from two independent experiments is shown in FIG. 5. Percentage was calculated as the percent relative to Ig kappa secretion levels from L-Wnt-3A cells treated with vehicle which correspond to 100%. Tested compound 10 increased the secretion of a particular protein (Wnt-3A) in L-Wnt-3A cells.

### Example B10—Secretion of EGF in Yeast

Saccharomyces cerevisiae stable expressing the recombinant human EGF protein (S.c-EGF) was obtained from ANGO Inc. S.c-EGF were cultured in 50 mL flask in SD-Leu-Glu medium (Sunrise Science Products) in agitation and at 30° C. At the final period of the exponential phase, 100 μL inocula were seeded in 48-well plates in 1 mL of complete medium with vehicle (0.1% DMSO) or 1 μM test compounds 10, 25, or 33 and incubated in continuous agitation (200 rpm) for 72° C. at 30° C. using a microplate spectrophotometer reader (Epoch, BioTek). After 72 hours treatments, culture media with cells were recovered in microtubes and centrifuged at 13,000 g for 5 minutes. Supernatants (SN) which contain secreted proteins were used to quantify the concentration of secreted human EGF by ELISA (ThermoFisher, Cat No. KHG0062) according to the manufacturer instructions. Final reaction was measured at 450 nm in the microplate Epoch (BioTek) and an internal calibration curve was used to calculate the amount of human EGF.

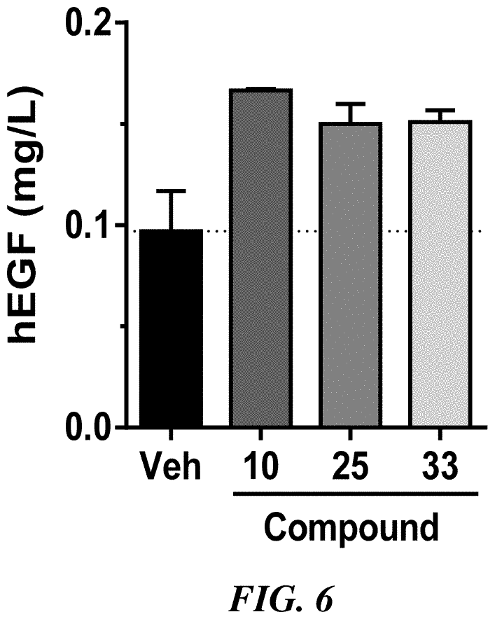

The amount of secreted human EGF protein by S.c-EGF treated with either vehicle or 1 μM test compounds is shown in FIG. 6. Tested compounds increased the secretion of a recombinant protein (hEGF) expressed in yeast.

All references throughout, such as publications, patents, patent applications and published patent applications, are incorporated herein by reference in their entireties.

1. A compound of formula (F-1) 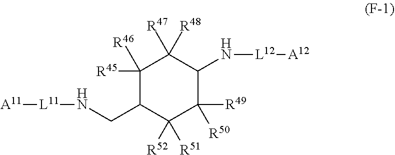
or a pharmaceutically acceptable salt thereof,
wherein:
R⁴⁵, R⁴⁶, R⁴⁷, R⁴⁸, R⁴⁹, R⁵⁰, R⁵¹, and R⁵², independently from each other, are selected from the group consisting of hydrogen, C₁-C₆ alkyl, C₁-C₆ haloalkyl, —C(O)OH, —C(O)O(C₁-C₆ alkyl), —C(O)O(C₁-C₆ haloalkyl), and halogen;
or, one of R⁴⁵, R⁴⁶, R⁴⁷, R⁴⁸, R⁴⁹, R⁵⁰, R⁵¹, and R⁵², and another one of R⁴⁵, R⁴⁶, R⁴⁷, R⁴⁸, R⁴⁹, R⁵⁰, R⁵¹, and R⁵², are taken together to form a C₁-C₆ alkylene moiety;
or, two geminal substituents selected from the group consisting of R⁴⁵, R⁴⁶, R⁴⁷, R⁴⁸, R⁴⁹, R⁵⁰, R⁵¹, and R⁵² are taken together to form an oxo group;
L¹¹ is selected from the group consisting of a bond, 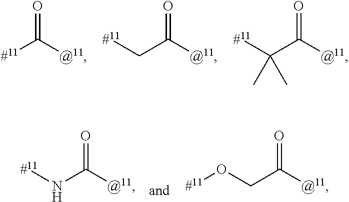
wherein #¹¹ represents to attachment point to A¹¹ and @¹¹ represents the attachment point to the remainder of the molecule;
L¹² is selected from the group consisting of 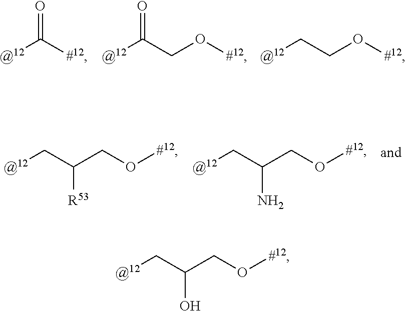
wherein #¹² represents to attachment point to A¹² and @¹² represents the attachment point to the remainder of the molecule;
R⁵³ is H, OH, or NH₂;
A¹¹ is selected from the group consisting of:
a substituent of formula (A¹¹-1) 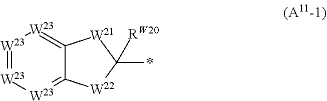
wherein * represents the attachment point to the remainder of the molecule;
W²¹ is selected from the group consisting of —C(R^{W21-1}R^{W21-2})—, —N(R^{W21-2})—, C(R^{W21-1}R^{W21-2})N(R^{W21-2})—, N(R^{W21-1})C(R^{W21-1}R^{W21-2})—, —C(R^{W21-1})═N—, —N═C(R^{W21-1})—, —O—, —C(R^{W21-1}R^{W21-1})O—, —OC(R^{W21-1}R^{W21-2})—, —S—, —C(R^{W21-1}R^{W21-1})S—, —SC(R^{W21-1}R^{W21-2})—, —C(R^{W21-1}R^{W21-1})C(R^{W21-1}R^{W21-2})—, and —CR^{W21-1}═CR^{W21-1}—,
wherein R^{W21-1} is H or R^{A1}, and R^{W21-2} is H or R^{A11};
W²² is selected from the group consisting of —C(R^{W22-1}R^{W22-2})—, —N(R^{W22-2})—, —C(R^{W22-1}R^{W22-1})N(R^{W22-2})—, —N(R^{W22-1})C(R^{W22-1}R^{W22-2})—, —C(R^{W22-1})═N—, —N═C(R^{W22-1})—, —O—, —C(R^{W22-1}R^{W22-1})O—, —OC(R^{W22-1}R^{W22-2})—, —S—, —C(R^{W22-1}R^{W22-1})S—, —SC(R^{W22-1}R^{W22-2})—, —C(R^{W22-1}R^{W22-1})C(R^{W22-1}R^{W22-2})—, and —CR^{W22-1}═CR^{W22-1}—,
wherein R^{W22-1} is H or R^{A1}, and R^{W22-2} is H or R^{A11};
W²³, independently at each occurrence, is CR^{W23} or N, wherein R^{W23} is H or R^{A11};
R^{W20} is hydrogen or R^{A11}, or R^{W20} and R^{W21-2} are taken together to form a double bond between the carbon atom bearing R^{W20} and the atom bearing R^{W21-2}, or R^{W20} and R^{W22-2} are taken together to form a double bond between the carbon atom bearing R^{W20} and the atom bearing R^{W21-2};
C₆-C₁₄ aryl optionally substituted with 1, 2, 3, 4, 5, 6, 7, 8, or 9 R^{A11} substituents; and
5-14 membered heteroaryl optionally substituted with 1, 2, 3, 4, 5, 6, 7, 8, or 9 R^{A11} substituents;
R^{A11}, independently at each occurrence, is selected from the group consisting of halogen, NO₂, C₁-C₆ alkyl, C₂-C₆ alkenyl, C₂-C₆ alkynyl, C₁-C₆ haloalkyl, OH, O(C₁-C₆ alkyl), O(C₁-C₆ haloalkyl), SH, S(C₁-C₆ alkyl), S(C₁-C₆ haloalkyl), NH₂, NH(C₁-C₆ alkyl), NH(C₁-C₆ haloalkyl), N(C₁-C₆ alkyl)₂, N(C₁-C₆ haloalkyl)₂, NR^{a}R^{b}, CN, C(O)OH, C(O)O(C₁-C₆ alkyl), C(O)O(C₁-C₆ haloalkyl), C(O)NH₂, C(O)NH(C₁-C₆ alkyl), C(O)NH(C₁-C₆ haloalkyl), C(O)N(C₁-C₆ alkyl)₂, C(O)N(C₁-C₆ haloalkyl)₂, C(O)NR^{a}R^{b}, S(O)₂OH, S(O)₂O(C₁-C₆ alkyl), S(O)₂O(C₁-C₆ haloalkyl), S(O)₂NH₂, S(O)₂NH(C₁-C₆ alkyl), S(O)₂NH(C₁-C₆ haloalkyl), S(O)₂N(C₁-C₆ alkyl)₂, S(O)₂N(C₁-C₆ haloalkyl)₂, S(O)₂NR^{a}R^{b}, OC(O)H, OC(O)(C₁-C₆ alkyl), OC(O)(C₁-C₆ haloalkyl), N(H)C(O)H, N(H)C(O)(C₁-C₆ alkyl), N(H)C(O)(C₁-C₆ haloalkyl), N(C₁-C₆ alkyl)C(O)H, N(C₁-C₆ alkyl)C(O)(C₁-C₆ alkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ alkyl), OS(O)₂(C₁-C₆ haloalkyl), N(H)S(O)₂(C₁-C₆ alkyl), N(H)S(O)₂(C₁-C₆ haloalkyl), N(C₁-C₆ alkyl)S(O)₂(C₁-C₆ alkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl); wherein R^{a} and R^{b} are taken together with the nitrogen atom to which they are attached to form a 3-10 membered heterocycle;
and
A¹² is selected from the group consisting of:
a substituent of formula (A¹²-1) 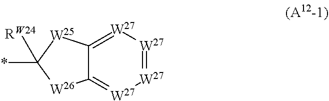
wherein * represents the attachment point to the remainder of the molecule;
W²⁵ is selected from the group consisting of —C(R^{W25-1}R^{W25-2})—, —N(R^{W25-2})—, —C(R^{W25-1}R^{W25-2})N(R^{W25-2})—, —N(R^{W25-1})C(R^{W25-1}R^{W25-2})—, —C(R^{W25-1})═N—, —N═C(R^{W25-1})—, —O—, —C(R^{W25-1}R^{W25-1})O—, —OC(R^{W25-1}R^{W25-2})—, —S—, —C(R^{W25-1}R^{W25-1})S—, —SC(R^{W25-1}R^{W25-2})—, —C(R^{W25-1}R^{W25-1})C(R^{W25-1}R^{W25-2})—, and —CR^{W25-1}═CR^{W25-1}—,
wherein R^{W25-1} is H or R^{A12}, and R^{W25-2} is H or R^{A12};
W²⁶ is selected from the group consisting of —C(R^{W26-1}R^{W26-2})—, —N(R^{W26-2})—, —C(R^{W26-1}R^{W26-1})N(R^{W26-2})—, —N(R^{W26-1})C(R^{W26-1}R^{W26-2})—, —C(R^{W26-1})═N—, —N═C(R^{W26-1})—, —O—, —C(R^{W26-1}R^{W26-1})O—, —OC(R^{W26-1}R^{W26-2})—, —S—, —C(R^{W26-1}R^{W26-1})S—, —SC(R^{W26-1}R^{W26-2})—, —C(R^{W26-1}R^{W26-1})C(R^{W26-1}R^{W26-2})—, and —CR^{W26-1}═CR^{W26-1}—,
wherein R^{W26-1} is H or R^{A12}, and R^{W26-2} is H or R^{A12};
W²⁷, independently at each occurrence, is CR^{W27} or N, wherein R^{W27} is H or R^{A12};
R^{W24} is hydrogen or R^{A12}, or R^{W24} and R^{W25-2} are taken together to form a double bond between the carbon atom bearing R^{W24} and the atom bearing R^{W25-2}, or R^{W24} and R^{W26-2} are taken together to form a double bond between the carbon atom bearing R^{W24} and the atom bearing R^{W26-2};
C₆-C₁₄ aryl optionally substituted with 1, 2, 3, 4, 5, 6, 7, 8, or 9 R^{A12} substituents; and
5-14 membered heteroaryl optionally substituted with 1, 2, 3, 4, 5, 6, 7, 8, or 9 R^{A12} substituents;
R^{A12}, independently at each occurrence, is selected from the group consisting of halogen, NO₂, C₁-C₆ alkyl, C₂-C₆ alkenyl, C₂-C₆ alkynyl, C₁-C₆ haloalkyl, OH, O(C₁-C₆ alkyl), O(C₁-C₆ haloalkyl), SH, S(C₁-C₆ alkyl), S(C₁-C₆ haloalkyl), NH₂, NH(C₁-C₆ alkyl), NH(C₁-C₆ haloalkyl), N(C₁-C₆ alkyl)₂, N(C₁-C₆ haloalkyl)₂, NR^{a}R^{b}, CN, C(O)OH, C(O)O(C₁-C₆ alkyl), C(O)O(C₁-C₆ haloalkyl), C(O)NH₂, C(O)NH(C₁-C₆ alkyl), C(O)NH(C₁-C₆ haloalkyl), C(O)N(C₁-C₆ alkyl)₂, C(O)N(C₁-C₆ haloalkyl)₂, C(O)NR^{a}R^{b}, S(O)₂OH, S(O)₂O(C₁-C₆ alkyl), S(O)₂O(C₁-C₆ haloalkyl), S(O)₂NH₂, S(O)₂NH(C₁-C₆ alkyl), S(O)₂NH(C₁-C₆ haloalkyl), S(O)₂N(C₁-C₆ alkyl)₂, S(O)₂N(C₁-C₆ haloalkyl)₂, S(O)₂NR^{a}R^{b}, OC(O)H, OC(O)(C₁-C₆ alkyl), OC(O)(C₁-C₆ haloalkyl), N(H)C(O)H, N(H)C(O)(C₁-C₆ alkyl), N(H)C(O)(C₁-C₆ haloalkyl), N(C₁-C₆ alkyl)C(O)H, N(C₁-C₆ alkyl)C(O)(C₁-C₆ alkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ alkyl), OS(O)₂(C₁-C₆ haloalkyl), N(H)S(O)₂(C₁-C₆ alkyl), N(H)S(O)₂(C₁-C₆ haloalkyl), N(C₁-C₆ alkyl)S(O)₂(C₁-C₆ alkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl); wherein R^{a} and R^{b} are taken together with the nitrogen atom to which they are attached to form a 3-10 membered heterocycle;
provided that
when L¹¹ is a bond, then A¹¹ is (A¹¹-1) optionally substituted by 1, 2, 3, 4, 5, 6, 7, 8, or 9 R^{A11} substituents;
when L¹¹ is 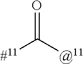
and L¹² is 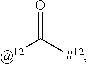
then A¹¹ is (A¹¹-1) substituted by 1, 2, 3, 4, 5, 6, 7, 8, or 9 R^{A11} substituents or A¹² is (A¹¹-1) substituted by 2, 3, 4, 5, 6, 7, 8, or 9 R^{A12} substituents;
and
when L¹¹ is 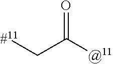
and L¹² is 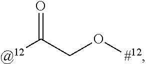
then A¹¹ is substituted by 1, 2, 3, 4, 5, 6, 7, 8, or 9 R^{A11} substituents.

2. A compound of formula (A-1) 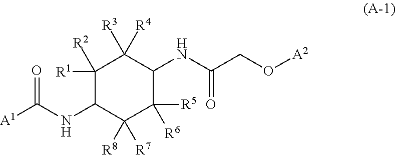
or a pharmaceutically acceptable salt thereof,
wherein:
R¹, R², R³, R⁴, R⁵, R⁶, R⁷, and R⁸, independently from each other, are selected from the group consisting of hydrogen, C₁-C₆ alkyl, C₁-C₆ haloalkyl, —C(O)OH, —C(O)O(C₁-C₆ alkyl), —C(O)O(C₁-C₆ haloalkyl), and halogen;
or, one of R¹, R², R³, R⁴, R⁵, R⁶, R⁷, and R⁸, and another one of R¹, R², R³, R⁴, R⁵, R⁶, R⁷, and R⁸, are taken together to form a C₁-C₆ alkylene moiety;
or, two geminal substituents selected from the group consisting of R¹, R², R³, R⁴, R⁵, R⁶, R⁷, and R⁸ are taken together to form an oxo group;
A¹ is selected from the group consisting of 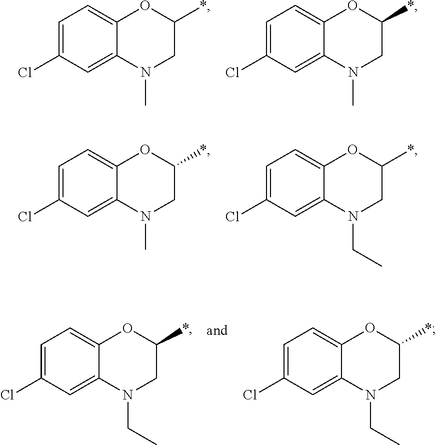
wherein * represents the attachment point to the remainder of the molecule;
and
A² is selected from the group consisting of:
C₆-C₁₄ aryl optionally substituted with 1, 2, 3, 4, 5, 6, 7, 8, or 9 R^{A2} substituents; and
5-14 membered heteroaryl optionally substituted with 1, 2, 3, 4, 5, 6, 7, 8, or 9 R^{A2} substituents;
R^{A2}, independently at each occurrence, is selected from the group consisting of halogen, NO₂, C₁-C₆ alkyl, C₂-C₆ alkenyl, C₂-C₆ alkynyl, C₁-C₆ haloalkyl, OH, O(C₁-C₆ alkyl), O(C₁-C₆ haloalkyl), SH, S(C₁-C₆ alkyl), S(C₁-C₆ haloalkyl), NH₂, NH(C₁-C₆ alkyl), NH(C₁-C₆ haloalkyl), N(C₁-C₆ alkyl)₂, N(C₁-C₆ haloalkyl)₂, NR^{a}R^{b}, CN, C(O)OH, C(O)O(C₁-C₆ alkyl), C(O)O(C₁-C₆ haloalkyl), C(O)NH₂, C(O)NH(C₁-C₆ alkyl), C(O)NH(C₁-C₆ haloalkyl), C(O)N(C₁-C₆ alkyl)₂, C(O)N(C₁-C₆ haloalkyl)₂, C(O)NR^{a}R^{b}, S(O)₂OH, S(O)₂O(C₁-C₆ alkyl), S(O)₂O(C₁-C₆ haloalkyl), S(O)₂NH₂, S(O)₂NH(C₁-C₆ alkyl), S(O)₂NH(C₁-C₆ haloalkyl), S(O)₂N(C₁-C₆ alkyl)₂, S(O)₂N(C₁-C₆ haloalkyl)₂, S(O)₂NR^{a}R^{b}, OC(O)H, OC(O)(C₁-C₆ alkyl), OC(O)(C₁-C₆ haloalkyl), N(H)C(O)H, N(H)C(O)(C₁-C₆ alkyl), N(H)C(O)(C₁-C₆ haloalkyl), N(C₁-C₆ alkyl)C(O)H, N(C₁-C₆ alkyl)C(O)(C₁-C₆ alkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ alkyl), OS(O)₂(C₁-C₆ haloalkyl), N(H)S(O)₂(C₁-C₆ alkyl), N(H)S(O)₂(C₁-C₆ haloalkyl), N(C₁-C₆ alkyl)S(O)₂(C₁-C₆ alkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl); wherein R^{a} and R^{b} are taken together with the nitrogen atom to which they are attached to form a 3-10 membered heterocycle.

3. A compound of formula (II) 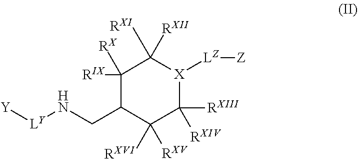
or a pharmaceutically acceptable salt thereof,
wherein:
X is N;
R^{IX}, R^{X}, R^{XI}, R^{XII}, R^{XIII}, R^{IV}, R^{XV}, and R^{XVI}, independently from each other, are selected from the group consisting of hydrogen, C₁-C₆ alkyl, C₁-C₆ haloalkyl, —C(O)OH, —C(O)O(C₁-C₆ alkyl), —C(O)O(C₁-C₆ haloalkyl), and halogen;
or, one of R^{IX}, R^{X}, R^{XI}, R^{XII}, R^{XIII}, R^{XIV}, R^{XV}, and R^{XVI}, and another one of R^{IX}, R^{X}, R^{XI}, R^{XII}, R^{XIII}, R^{XIV}, R^{XV}, and R^{XVI}, are taken together to form a C₁-C₆ alkylene moiety;
or, two geminal substituents selected from the group consisting of R^{IX}, R^{X}, R^{XI}, R^{XII}, R^{XIII}, R^{XIV}, R^{XV}, and R^{XVI} are taken together to form an oxo group;
L^{Y} is 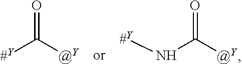
wherein #^{Y} represents the attachment point to Y and @^{Y} represents the attachment point to the remainder of the molecule;
L^{Z} is selected from the group consisting of 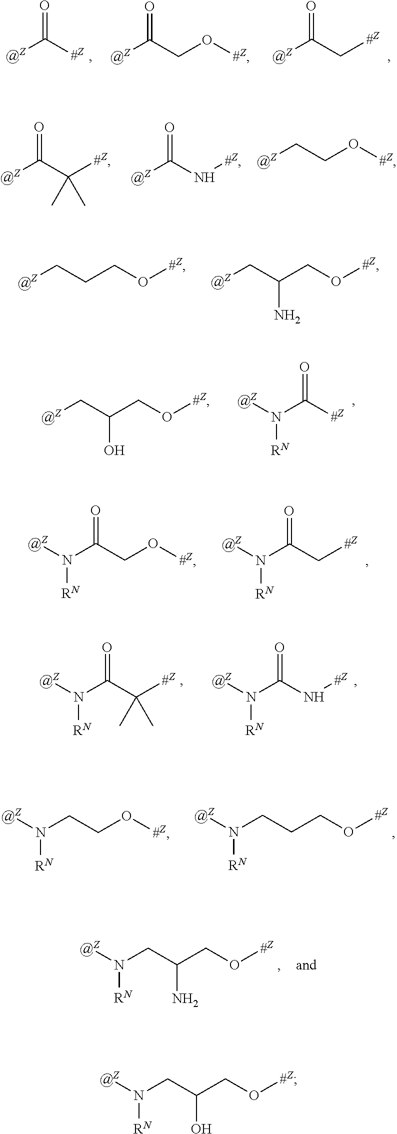
wherein #^{Z} represents the attachment point to Z and @^{Z} represents the attachment point to the remainder of the molecule;
R^{N}, independently at each occurrence, is selected from the group consisting of hydrogen, C₁-C₆ alkyl, and C₁-C₆ haloalkyl,
Y is a substituent of formula (Y-I) 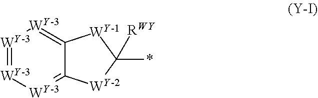
wherein * represents the attachment point to the remainder of the molecule;
W^{Y-1} is selected from the group consisting of —C(R^{WY-1-1}R^{WY-1-2})—, —N(R^{WY-1-2})—, —C(R^{WY-1-1}R^{WY-1-1})N(R^{WY-1-2})—, —N(R^{WY-1-1})C(R^{WY-1-1}R^{WY-1-2})—, —C(R^{WY-1-1})═N—, —N═C(R^{WY-1-1})—, —O—, —C(R^{WY-1-1}R^{WY-1-1})O—, —OC(R^{WY-1-1}R^{WY-1-2})—, —S—, —C(R^{WY-1-1}R^{WY-1-1})S—, —SC(R^{WY-1-1}R^{WY-1-2})—, —C(R^{WY-1-1}R^{WY-1-1})C(R^{WY-1-1}R^{WY-1-2}) and —CR^{WY-1-1}═CR^{WY-1-1}—,
wherein R^{WY-1-1} is H or R^{Y}, and R^{WY-1-2} is H or R^{Y};
W^{Y-2} is selected from the group consisting of —C(R^{WY-2-1}R^{WY-2-2})—, —N(R^{WY-2-2})—, —C(R^{WY-2-1}R^{WY-2-1})N(R^{WY-2-2})—, —N(R^{WY-2-1})C(R^{WY-2-1}R^{WY-2-2})—, —C(R^{WY-2-1})═N—, —N═C(R^{WY-2-1})—, —O—, —C(R^{WY-2-1}R^{WY-2-1})O—, —OC(R^{WY-2-1}R^{WY-2-2})—, —S—, —C(R^{WY-2-1}R^{WY-2-1})S—, —SC(R^{WY-2-1}R^{WY-2-2})—, —C(R^{WY-2-1}R^{WY-2-1})C(R^{WY-2-1}R^{WY-2-2}) and —CR^{WY-2-1}═CR^{WY-2-1}—,
wherein R^{WY-2-1} is H or R^{Y}, and R^{WY-2-2} is H or R^{Y};
W^{Y-3}, independently at each occurrence, is CR^{WY-3} or N, wherein R^{WY-3} is H or R^{Y};
R^{WY} is hydrogen or R^{Y}, or R^{WY} and R^{WY-1-2} are taken together to form a double bond between the carbon atom bearing R^{WY} and the atom bearing R^{WY-1-2}, or R^{WY} and R^{WY-2-2} are taken together to form a double bond between the carbon atom bearing R^{WY} and the atom bearing R^{WY-2-2};
C₆-C₁₄ aryl optionally substituted with 1, 2, 3, 4, 5, 6, 7, 8, or 9 R^{Y} substituents; and
5-14 membered heteroaryl optionally substituted with 1, 2, 3, 4, 5, 6, 7, 8, or 9 R^{Y} substituents;
R^{Y}, independently at each occurrence, is selected from the group consisting of halogen, NO₂, C₁-C₆ alkyl, C₂-C₆ alkenyl, C₂-C₆ alkynyl, C₁-C₆ haloalkyl, OH, O(C₁-C₆ alkyl), O(C₁-C₆ haloalkyl), SH, S(C₁-C₆ alkyl), S(C₁-C₆ haloalkyl), NH₂, NH(C₁-C₆ alkyl), NH(C₁-C₆ haloalkyl), N(C₁-C₆ alkyl)₂, N(C₁-C₆ haloalkyl)₂, NR^{a}R^{b}, CN, C(O)OH, C(O)O(C₁-C₆ alkyl), C(O)O(C₁-C₆ haloalkyl), C(O)NH₂, C(O)NH(C₁-C₆ alkyl), C(O)NH(C₁-C₆ haloalkyl), C(O)N(C₁-C₆ alkyl)₂, C(O)N(C₁-C₆ haloalkyl)₂, C(O)NR^{a}R^{b}, S(O)₂OH, S(O)₂O(C₁-C₆ alkyl), S(O)₂O(C₁-C₆ haloalkyl), S(O)₂NH₂, S(O)₂NH(C₁-C₆ alkyl), S(O)₂NH(C₁-C₆ haloalkyl), S(O)₂N(C₁-C₆ alkyl)₂, S(O)₂N(C₁-C₆ haloalkyl)₂, S(O)₂NR^{a}R^{b}, OC(O)H, OC(O)(C₁-C₆ alkyl), OC(O)(C₁-C₆ haloalkyl), N(H)C(O)H, N(H)C(O)(C₁-C₆ alkyl), N(H)C(O)(C₁-C₆ haloalkyl), N(C₁-C₆ alkyl)C(O)H, N(C₁-C₆ alkyl)C(O)(C₁-C₆ alkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ alkyl), OS(O)₂(C₁-C₆ haloalkyl), N(H)S(O)₂(C₁-C₆ alkyl), N(H)S(O)₂(C₁-C₆ haloalkyl), N(C₁-C₆ alkyl)S(O)₂(C₁-C₆ alkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl); wherein R^{a} and R^{b} are taken together with the nitrogen atom to which they are attached to form a 3-10 membered heterocycle;
and
Z is selected from the group consisting of:
a substituent of formula (Z-I) 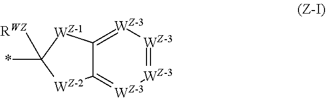
wherein * represents the attachment point to the remainder of the molecule;
W^{Z-1} is selected from the group consisting of —C(R^{WZ-1-1}R^{WZ-1-2})—, —N(R^{WZ-1-2})—, —C(R^{WZ-1-1}R^{WZ-1-2})N(R^{WZ-1-2})—, —N(R^{WZ-1-1})C(R^{WZ-1-1}R^{WZ-1-2})—, —C(R^{WZ-1-1})═N—, —N═C(R^{WZ-1-1})—, —O—, —C(R^{WZ-1-1}R^{WZ-1-1})O—, —OC(R^{WZ-1-1}R^{WZ-1-2})—, —S—, —C(R^{WZ-1-1}R^{WZ-1-1})S—, —SC(R^{WZ-1-1}R^{WZ-1-2})—, —C(R^{WZ-1-1}R^{WZ-1-1})C(R^{WZ-1-1}R^{WZ-1-2})—, and —CR^{WZ-1-1}═CR^{WZ-1-1}—,
wherein R^{WZ-1-1} is H or R^{Z}, and R^{WZ-1-2} is H or R^{Z};
W^{Z-2} is selected from the group consisting of —C(R^{WZ-2-1}R^{WZ-2-2})—, —N(R^{WZ-2-2})—, —C(R^{WZ-2-1}R^{WZ-2-1})N(R^{WZ-2-2})—, —N(R^{WZ-2-1})C(R^{WZ-2-1}R^{WZ-2-2})—, —C(R^{WZ-2-1})═N—, —N═C(R^{WZ-2-1})—, —O—, —C(R^{WZ-2-1}R^{WZ-2-1})O—, —OC(R^{WZ-2-1}R^{WZ-2-2})—, —S—, —C(R^{WZ-2-1}R^{WZ-2-1})S—, —SC(R^{WZ-2-1}R^{WZ-2-2})—, —C(R^{WZ-2-1}R^{WZ-2-1})C(R^{WZ-2-1}R^{WZ-2-2})—, and —CR^{WZ-2-1}═CR^{WZ-2-1},
wherein R^{WZ-2-1} is H or R^{Z}, and R^{WZ-2-2} is H or R^{Z};
W^{Z-3}, independently at each occurrence, is CR^{WZ-3} or N, wherein R^{WZ-3} is H or R^{Z};
R^{WZ} is hydrogen or R^{Z}, or R^{WZ} and R^{WZ-1-2} are taken together to form a double bond between the carbon atom bearing R^{WZ} and the atom bearing R^{WZ-1-2}, or R^{WZ} and R^{WZ-2-2} are taken together to form a double bond between the carbon atom bearing R^{WZ} and the atom bearing R^{WZ-2-2};
C₆-C₁₄ aryl optionally substituted with 1, 2, 3, 4, 5, 6, 7, 8, or 9 R^{Z} substituents; and
5-14 membered heteroaryl optionally substituted with 1, 2, 3, 4, 5, 6, 7, 8, or 9 R^{Z} substituents;
R^{Z}, independently at each occurrence, is selected from the group consisting of halogen, NO₂, C₁-C₆ alkyl, C₂-C₆ alkenyl, C₂-C₆ alkynyl, C₁-C₆ haloalkyl, OH, O(C₁-C₆ alkyl), O(C₁-C₆ haloalkyl), SH, S(C₁-C₆ alkyl), S(C₁-C₆ haloalkyl), NH₂, NH(C₁-C₆ alkyl), NH(C₁-C₆ haloalkyl), N(C₁-C₆ alkyl)₂, N(C₁-C₆ haloalkyl)₂, NR^{a}R^{b}, CN, C(O)OH, C(O)O(C₁-C₆ alkyl), C(O)O(C₁-C₆ haloalkyl), C(O)NH₂, C(O)NH(C₁-C₆ alkyl), C(O)NH(C₁-C₆ haloalkyl), C(O)N(C₁-C₆ alkyl)₂, C(O)N(C₁-C₆ haloalkyl)₂, C(O)NR^{a}R^{b}, S(O)₂OH, S(O)₂O(C₁-C₆ alkyl), S(O)₂O(C₁-C₆ haloalkyl), S(O)₂NH₂, S(O)₂NH(C₁-C₆ alkyl), S(O)₂NH(C₁-C₆ haloalkyl), S(O)₂N(C₁-C₆ alkyl)₂, S(O)₂N(C₁-C₆ haloalkyl)₂, S(O)₂NR^{a}R^{b}, OC(O)H, OC(O)(C₁-C₆ alkyl), OC(O)(C₁-C₆ haloalkyl), N(H)C(O)H, N(H)C(O)(C₁-C₆ alkyl), N(H)C(O)(C₁-C₆ haloalkyl), N(C₁-C₆ alkyl)C(O)H, N(C₁-C₆ alkyl)C(O)(C₁-C₆ alkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ alkyl), OS(O)₂(C₁-C₆ haloalkyl), N(H)S(O)₂(C₁-C₆ alkyl), N(H)S(O)₂(C₁-C₆ haloalkyl), N(C₁-C₆ alkyl)S(O)₂(C₁-C₆ alkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl); wherein R^{a} and R^{b} are taken together with the nitrogen atom to which they are attached to form a 3-10 membered heterocycle;
provided that when L^{Y} is 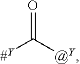
Y is (Y-I);
when L^{Y} is 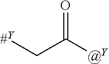
and L^{Z} is 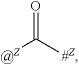
then Y is (Y-I) substituted by 1, 2, 3, 4, 5, 6, 7, 8, or 9 R^{Y} substituents or Z is (Z-I) substituted by 2, 3, 4, 5, 6, 7, 8, or 9 R^{Z} substituents;
and
when L^{Y} is 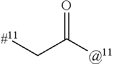
and L^{Z} is 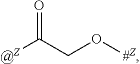
then Y is substituted by 1, 2, 3, 4, 5, 6, 7, 8, or 9 R^{Y} substituents.

4. A compound of formula (III) 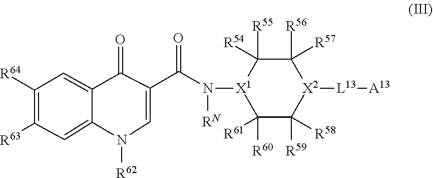
or a salt thereof,
wherein:
X¹ is N or CR^{X1};
X² is N or CR^{X2};
when present, R^{X1} is selected from the group consisting of hydrogen, C₁-C₆ alkyl, C₁-C₆ haloalkyl, —C(O)OH, —C(O)O(C₁-C₆ alkyl), —C(O)O(C₁-C₆ haloalkyl), and halogen;
when present, R² is selected from the group consisting of hydrogen, C₁-C₆ alkyl, C₁-C₆ haloalkyl, —C(O)OH, —C(O)O(C₁-C₆ alkyl), —C(O)O(C₁-C₆ haloalkyl), and halogen;
R⁵⁴, R⁵⁵, R⁵⁶, R⁵⁷, R⁵⁸, R⁵⁹, R⁶⁰, and R⁶¹, independently from each other, are selected from the group consisting of hydrogen, C₁-C₆ alkyl, C₁-C₆ haloalkyl, —C(O)OH, —C(O)O(C₁-C₆ alkyl), —C(O)O(C₁-C₆ haloalkyl), and halogen;
or, one of R⁵⁴, R⁵⁵, R⁵⁶, R⁵⁷, R⁵⁸, R⁵⁹, R⁶⁰, and R⁶¹, and another one of R⁵⁴, R⁵⁵, R⁵⁶, R⁵⁷, R⁵⁸, R⁵⁹, R⁶⁰, and R⁶¹, are taken together to form a C₁-C₆ alkylene moiety;
or, two geminal substituents selected from the group consisting of R⁵⁴, R⁵⁵, R⁵⁶, R⁵⁷, R⁵⁸, R⁵⁹, R⁶⁰, and R⁶¹ are taken together to form an oxo group;
or, two of R⁵⁴, R⁵⁵, R⁵⁶, R⁵⁷, R⁵⁸, R⁵⁹, R⁶⁰, R⁶¹, Rx when present, and R^{X2}, when present, are taken together to form a C₁-C₆ alkylene moiety;
R⁶³ and R⁶⁴, independently from each other, are selected from the group consisting of hydrogen, halogen, NO₂, C₁-C₆ alkyl, C₂-C₆ alkenyl, C₂-C₆ alkynyl, C₁-C₆ haloalkyl, —OH, —O(C₁-C₆ alkyl), —O(C₁-C₆ haloalkyl), —SH, —S(C₁-C₆ alkyl), —S(C₁-C₆ haloalkyl), —NH₂, —NH(C₁-C₆ alkyl), —NH(C₁-C₆ haloalkyl), —N(C₁-C₆ alkyl)₂, —N(C₁-C₆ haloalkyl)₂, —NR^{B-a}R^{B-b}, —CN, —C(O)OH, —C(O)O(C₁-C₆ alkyl), —C(O)O(C₁-C₆ haloalkyl), —C(O)NH₂, —C(O)NH(C₁-C₆ alkyl), —C(O)NH(C₁-C₆ haloalkyl), —C(O)N(C₁-C₆ alkyl)₂, —C(O)N(C₁-C₆ haloalkyl)₂, —C(O)NR^{B-a}R^{B-b}, —S(O)₂OH, —S(O)₂O(C₁-C₆ alkyl), —S(O)₂O(C₁-C₆ haloalkyl), —S(O)₂NH₂, —S(O)₂NH(C₁-C₆ alkyl), —S(O)₂NH(C₁-C₆ haloalkyl), —S(O)₂N(C₁-C₆ alkyl)₂, —S(O)₂N(C₁-C₆ haloalkyl)₂, —S(O)₂NR^{B-a}R^{B-b}, —OC(O)H, —OC(O)(C₁-C₆ alkyl), —OC(O)(C₁-C₆ haloalkyl), —N(H)C(O)H, —N(H)C(O)(C₁-C₆ alkyl), —N(H)C(O)(C₁-C₆ haloalkyl), —N(C₁-C₆ alkyl)C(O)H, —N(C₁-C₆ alkyl)C(O)(C₁-C₆ alkyl), —N(C₁-C₆ alkyl)C(O)(C₁-C₆ haloalkyl), —N(C₁-C₆ haloalkyl)C(O)H, —N(C₁-C₆ haloalkyl)C(O)(C₁-C₆ alkyl), —N(C₁-C₆ haloalkyl)C(O)(C₁-C₆ haloalkyl), —OS(O)₂(C₁-C₆ alkyl), —OS(O)₂(C₁-C₆ haloalkyl), —N(H)S(O)₂(C₁-C₆ alkyl), —N(H)S(O)₂(C₁-C₆ haloalkyl), —N(C₁-C₆ alkyl)S(O)₂(C₁-C₆ alkyl), —N(C₁-C₆ alkyl)S(O)₂(C₁-C₆ haloalkyl), —N(C₁-C₆ haloalkyl)S(O)₂(C₁-C₆ alkyl), and —N(C₁-C₆ haloalkyl)S(O)₂(C₁-C₆ haloalkyl);
wherein R^{B-a} and R^{B-b} are taken together with the nitrogen atom to which they are attached to form a 3-10 membered heterocycle;
R⁶² is selected from the group consisting of halogen, NO₂, C₁-C₆ alkyl, C₂-C₆ alkenyl, C₂-C₆ alkynyl, —(C₁-C₆ alkylene)-(C₆-C₁₄ aryl optionally substituted with 1, 2, 3, 4, 5, 6, 7, 8, or 9 R^{A13} substituents), —(C₁-C₆ alkylene)-(5-14 membered heteroaryl optionally substituted with 1, 2, 3, 4, 5, 6, 7, 8, or 9 R^{A13} substituents), C₁-C₆ haloalkyl, —OH, —O(C₁-C₆ alkyl), —O(C₁-C₆ haloalkyl), —(C₁-C₆ alkylene)-OH, —(C₁-C₆ alkylene)-O—(C₁-C₆ alkyl), —(C₁-C₆ alkylene)-O—(C₁-C₆ haloalkyl), —SH, —S(C₁-C₆ alkyl), —S(C₁-C₆ haloalkyl), —NH₂, —NH(C₁-C₆ alkyl), —NH(C₁-C₆ haloalkyl), —N(C₁-C₆ alkyl)₂, —N(C₁-C₆ haloalkyl)₂, —CN, —C(O)OH, —C(O)O(C₁-C₆ alkyl), —C(O)O(C₁-C₆ haloalkyl), —C(O)NH₂, —C(O)NH(C₁-C₆ alkyl), —C(O)NH(C₁-C₆ haloalkyl), —C(O)N(C₁-C₆ alkyl)₂, —C(O)N(C₁-C₆ haloalkyl)₂, —C(O)NR^{62-a}R^{62-b}, —S(O)₂H, —S(O)₂(C₁-C₆ alkyl), —S(O)₂O(C₁-C₆ haloalkyl), —S(O)₂NH₂, —S(O)₂NH(C₁-C₆ alkyl), —S(O)₂NH(C₁-C₆ haloalkyl), —S(O)₂N(C₁-C₆ alkyl)₂, —S(O)₂N(C₁-C₆ haloalkyl)₂, —S(O)₂NR^{62-a}R^{62-b}, —OC(O)H, —OC(O)(C₁-C₆ alkyl), —OC(O)(C₁-C₆ haloalkyl), —N(H)C(O)H, —N(H)C(O)(C₁-C₆ alkyl), —N(H)C(O)(C₁-C₆ haloalkyl), —N(C₁-C₆ alkyl)C(O)H, —N(C₁-C₆ alkyl)C(O)(C₁-C₆ alkyl), —N(C₁-C₆ alkyl)C(O)(C₁-C₆ haloalkyl), —N(C₁-C₆ haloalkyl)C(O)H, —N(C₁-C₆ haloalkyl)C(O)(C₁-C₆ alkyl), —N(C₁-C₆ haloalkyl)C(O)(C₁-C₆ haloalkyl), —OS(O)₂(C₁-C₆ alkyl), —OS(O)₂(C₁-C₆ haloalkyl), —N(H)S(O)₂(C₁-C₆ alkyl), —N(H)S(O)₂(C₁-C₆ haloalkyl), —N(C₁-C₆ alkyl)S(O)₂(C₁-C₆ alkyl), —N(C₁-C₆ alkyl)S(O)₂(C₁-C₆ haloalkyl), —N(C₁-C₆ haloalkyl)S(O)₂(C₁-C₆ alkyl), and —N(C₁-C₆ haloalkyl)S(O)₂(C₁-C₆ haloalkyl);
wherein R^{62-a} and R^{62-b} are taken together with the nitrogen atom to which they are attached to form a 3-10 membered heterocycle;
L¹³ is a linker selected from the group consisting of @¹³—C₁-C₆ alkylene-#¹³, @¹³—NR^{N}—(C₁-C₆ alkylene)-#¹³, @¹³—NR^{N}—NR^{N}—(C₁-C₆ alkylene)-#¹³ @¹³—CH₂—NR^{N}—(C₁-C₆ alkylene)-#¹³, @¹³—CH₂—NR^{N}—NR^{N}—(C₁-C₆ alkylene)-#¹³, @¹³—NR^{N}—(C₁-C₆ alkylene)-O-#¹³, @¹³—NR^{N}—NR^{N}—(C₁-C₆ alkylene)-O-#¹³, @³—CH₂—NR^{N}—(C₁-C₆ alkylene)-O-#¹³, @¹³—CH₂—NR^{N}—NR^{N}—(C₁-C₆ alkylene)-O-#¹³, and @¹³—(C₁-C₆ alkylene)-O-#¹³;
wherein @¹³ represents the attachment point to X² and #¹³ represents the attachment point to A¹³;
the C₁-C₆ alkylene moiety of each of the @¹³—C₁-C₆ alkylene-#¹³, @¹³—NR^{N}—(C₁-C₆ alkylene)-#¹³, @¹³—NR^{N}—NR^{N}—(C₁-C₆ alkylene)-#¹³, @¹³—CH₂—NR^{N}—(C₁-C₆ alkylene)-#¹³, @¹³—CH₂—NR^{N}—NR^{N}—(C₁-C₆ alkylene)-#¹³, @¹³—NR^{N}—(C₁-C₆ alkylene)-O-#¹³, @¹³—NR^{N}—NR^{N}—(C₁-C₆ alkylene)-O-#¹³, @¹³—CH₂—NR^{N}—(C₁-C₆ alkylene)-O-#¹³, @¹³—CH₂—NR^{N}—NR^{N}—(C₁-C₆ alkylene)-O-#¹³, and @¹³—(C₁-C₆ alkylene)-O-#¹³ is optionally substituted with 1 to 12 R⁶⁶;
R^{N}, independently at each occurrence, is selected from the group consisting of hydrogen, C₁-C₆ alkyl, and C₁-C₆ haloalkyl,
R⁶⁶, independently at each occurrence, is selected from the group consisting of oxo, halogen, C₁-C₆ alkyl, C₂-C₆ alkenyl, C₂-C₆ alkynyl, C₁-C₆ haloalkyl, —OH, —O(C₁-C₆ alkyl), —O(C₁-C₆ haloalkyl), —SH, —S(C₁-C₆ alkyl), —S(C₁-C₆ haloalkyl), —NH₂, —NH(C₁-C₆ alkyl), —NH(C₁-C₆ haloalkyl), —N(C₁-C₆ alkyl)₂, —N(C₁-C₆ haloalkyl)₂, —NR^{B-a}R^{B-b}, —CN, —C(O)OH, —C(O)O(C₁-C₆ alkyl), —C(O)O(C₁-C₆ haloalkyl), —C(O)NH₂, —C(O)NH(C₁-C₆ alkyl), —C(O)NH(C₁-C₆ haloalkyl), —C(O)N(C₁-C₆ alkyl)₂, —C(O)N(C₁-C₆ haloalkyl)₂, —C(O)NR^{B-a}R^{B-b}, —S(O)₂OH, —S(O)₂O(C₁-C₆ alkyl), —S(O)₂O(C₁-C₆ haloalkyl), —S(O)₂NH₂, —S(O)₂NH(C₁-C₆ alkyl), —S(O)₂NH(C₁-C₆ haloalkyl), —S(O)₂N(C₁-C₆ alkyl)₂, —S(O)₂N(C₁-C₆ haloalkyl)₂, —S(O)₂NR^{B-a}R^{B-b}, —OC(O)H, —OC(O)(C₁-C₆ alkyl), —OC(O)(C₁-C₆ haloalkyl), —N(H)C(O)H, —N(H)C(O)(C₁-C₆ alkyl), —N(H)C(O)(C₁-C₆ haloalkyl), —N(C₁-C₆ alkyl)C(O)H, —N(C₁-C₆ alkyl)C(O)(C₁-C₆ alkyl), —N(C₁-C₆ alkyl)C(O)(C₁-C₆ haloalkyl), —N(C₁-C₆ haloalkyl)C(O)H, —N(C₁-C₆ haloalkyl)C(O)(C₁-C₆ alkyl), —N(C₁-C₆ haloalkyl)C(O)(C₁-C₆ haloalkyl), —OS(O)₂(C₁-C₆ alkyl), —OS(O)₂(C₁-C₆ haloalkyl), —N(H)S(O)₂(C₁-C₆ alkyl), —N(H)S(O)₂(C₁-C₆ haloalkyl), —N(C₁-C₆ alkyl)S(O)₂(C₁-C₆ alkyl), —N(C₁-C₆ alkyl)S(O)₂(C₁-C₆ haloalkyl), —N(C₁-C₆ haloalkyl)S(O)₂(C₁-C₆ alkyl), and —N(C₁-C₆ haloalkyl)S(O)₂(C₁-C₆ haloalkyl);
A¹³ is selected from the group consisting of:
a substituent of formula (A¹³-1) 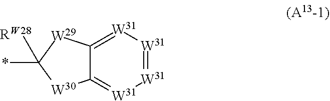
W²⁹ is selected from the group consisting of —C(R^{W29-1}R^{W29-2})—, —N(R^{W29-2})—, —C(R^{W29-1}R^{W29-1})N(R^{W29-2})—, —N(R^{W29-1})C(R^{W29-1}R^{W29-2})—, —C(R^{W29-1})═N—, —N═C(R^{W29-1})—, —O—, —C(R^{W29-1}R^{W29-1})O—, —OC(R^{W29-1}R^{W29-2})—, —O—, —C(R^{W29-1}R^{W29-1})S—, —SC(R^{W29-1}R^{W29-2})—, —C(R^{W29-1}R^{W29-1})C(R^{W29-1}R^{W29-2})—, and —CR^{W29-1}═CR^{W29-1}—,
wherein R^{W29-1} is H or R^{A}, and R^{W29-2} is H or R^{A3};
W³⁰ is selected from the group consisting of —C(R^{W30-1}R^{W30-2}), N(R^{W30-2}—, —C(R^{W30-1}R^{W30-1})N(R^{W30-2})—, —N(R^{W30-1})C(R^{W30-1}R^{W30-2})—, —C(R^{W30-1})═N—, —N═C(R^{W30-1})—, —O—, —C(R^{W30-1}R^{W30-1})O—, —OC(R^{W30-1}R^{W30-2}), —S—, —C(R^{W30-1}R^{W30-1})S—, —SC(R^{W30-1}R^{W30-2})—, —C(R^{W30-1}R^{W30-1})C(R^{W30-1}R^{W30-2})—, and —CR^{W30-1}═CR^{W30-1}—,
wherein R^{W30-1} is H or R^{A13}, and R^{W30-2} is H or R^{A13};
W³¹, independently at each occurrence, is CR^{W31} or N, wherein R^{W31} is H or R^{A13};
R^{W28} is hydrogen or R^{A13}, or R^{W28} and R^{W29-2} are taken together to form a double bond between the carbon atom bearing R^{W28} and the atom bearing R^{W29-2}, or R^{W28} and R^{W30-2} are taken together to form a double bond between the carbon atom bearing R^{W28} and the atom bearing R^{W30-2};
C₆-C₁₄ aryl optionally substituted with 1, 2, 3, 4, 5, 6, 7, 8, or 9 R^{A13} substituents; and
5-14 membered heteroaryl optionally substituted with 1, 2, 3, 4, 5, 6, 7, 8, or 9 R^{A13} substituents;
R^{A13}, independently at each occurrence, is selected from the group consisting of halogen, NO₂, C₁-C₆ alkyl, C₂-C₆ alkenyl, C₂-C₆ alkynyl, C₁-C₆ haloalkyl, —OH, —O(C₁-C₆ alkyl), —O(C₁-C₆ haloalkyl), —SH, —S(C₁-C₆ alkyl), —S(C₁-C₆ haloalkyl), —NH₂, —NH(C₁-C₆ alkyl), —NH(C₁-C₆ haloalkyl), —N(C₁-C₆ alkyl)₂, —N(C₁-C₆ haloalkyl)₂, —NR^{A13-a}R^{A13-b}, —CN, —C(O)OH, —C(O)O(C₁-C₆ alkyl), —C(O)O(C₁-C₆ haloalkyl), —C(O)NH₂, —C(O)NH(C₁-C₆ alkyl), —C(O)NH(C₁-C₆ haloalkyl), —C(O)N(C₁-C₆ alkyl)₂, —C(O)N(C₁-C₆ haloalkyl)₂, —C(O)NR^{A13-a}R^{A13-b}, —S(O)₂H, —S(O)₂(C₁-C₆ alkyl), —S(O)₂O(C₁-C₆ haloalkyl), —S(O)₂NH₂, —S(O)₂NH(C₁-C₆ alkyl), —S(O)₂NH(C₁-C₆ haloalkyl), —S(O)₂N(C₁-C₆ alkyl)₂, —S(O)₂N(C₁-C₆ haloalkyl)₂, —S(O)₂NR^{A13-a}R^{A13-b}, —OC(O)H, —OC(O)(C₁-C₆ alkyl), —OC(O)(C₁-C₆ haloalkyl), —N(H)C(O)H, —N(H)C(O)(C₁-C₆ alkyl), —N(H)C(O)(C₁-C₆ haloalkyl), —N(C₁-C₆ alkyl)C(O)H, —N(C₁-C₆ alkyl)C(O)(C₁-C₆ alkyl), —N(C₁-C₆ alkyl)C(O)(C₁-C₆ haloalkyl), —N(C₁-C₆ haloalkyl)C(O)H, —N(C₁-C₆ haloalkyl)C(O)(C₁-C₆ alkyl), —N(C₁-C₆ haloalkyl)C(O)(C₁-C₆ haloalkyl), —OS(O)₂(C₁-C₆ alkyl), —OS(O)₂(C₁-C₆ haloalkyl), —N(H)S(O)₂(C₁-C₆ alkyl), —N(H)S(O)₂(C₁-C₆ haloalkyl), —N(C₁-C₆ alkyl)S(O)₂(C₁-C₆ alkyl), —N(C₁-C₆ alkyl)S(O)₂(C₁-C₆ haloalkyl), —N(C₁-C₆ haloalkyl)S(O)₂(C₁-C₆ alkyl), and —N(C₁-C₆ haloalkyl)S(O)₂(C₁-C₆ haloalkyl);
wherein R^{A13-a} and R^{A13-b} are taken together with the nitrogen atom to which they are attached to form a 3-10 membered heterocycle;
provided that when X² is N, then L¹³ is a linker selected from the group consisting of @¹³—C₁-C₆ alkylene-#¹³, @¹³-NR^{N}—(C₁-C₆ alkylene)-#¹³ @¹³—NR^{N}—(C₁-C₆ alkylene)-O-#¹³, and @¹³—(C₁-C₆ alkylene)-O-#¹³; and further provided that when X¹ is CH, X² is N, R⁶² is methyl, and L¹³ is @¹³—CH₂-#¹³, then A¹³ is then A¹³ is (A¹³-1), C₆-C₁₄ aryl optionally substituted with 1, 2, 3, 4, 5, 6, 7, 8, or 9 R^{A13} substituents, or 5-14 membered heteroaryl substituted with 1, 2, 3, 4, 5, 6, 7, 8, or 9 R^{A13} substituents.

5. A compound selected from the group consisting of a compound of Table 1, or a pharmaceutically acceptable salt thereof.

6. A compound selected from the group consisting of compounds 1 to 34, or a pharmaceutically acceptable salt thereof.

7. A pharmaceutical composition comprising a compound of claim 1, or a pharmaceutically acceptable salt thereof, and a pharmaceutically acceptable carrier.

8. A method for enhancing protein synthesis in a living organism, comprising administering to the living organism an effective amount of a compound of claim 1, or a salt thereof.

9. A method for accelerating growth of a plant, comprising administering to the plant an effective amount of a compound of claim 1, or a salt thereof.

10. A method for improving protein yield or quality in a plant, comprising administering to the plant an effective amount of a compound of claim 1, or a salt thereof.

11. The method of claim 10, wherein the plant is selected from soybean, sunflower, grain legume, rice, wheat germ, maize, tobacco, a cereal, and a lupin crop.

12. A method of treating a disease or disorder mediated by an integrated stress response (ISR) pathway in an individual in need thereof comprising administering to the individual a therapeutically effective amount of a compound of claim 1, or a pharmaceutically acceptable salt thereof.

13. The method of claim 12, wherein the compound, the pharmaceutically acceptable salt, or the pharmaceutical composition is administered in combination with a therapeutically effective amount of one or more additional anti-cancer agents.

14. The method of claim 12, wherein the disease or disorder is mediated by phosphorylation of eIF2α and/or the guanine nucleotide exchange factor (GEF) activity of eIF2B.

15. The method of claim 12, wherein the disease or disorder is mediated by a decrease in protein synthesis.

16. The method of claim 12, wherein the disease or disorder is mediated by the expression of ATF4, CHOP or BACE-1.

17. The method of claim 12, wherein the disease or disorder is a neurodegenerative disease, an inflammatory disease, an autoimmune disease, a metabolic syndrome, a cancer, a vascular disease, an ocular disease, a musculoskeletal disease, or a genetic disorder.

18. The method of claim 17, wherein the disease is vanishing white matter disease, childhood ataxia with CNS hypomyelination, intellectual disability syndrome, Alzheimer's disease, prion disease, Creutzfeldt-Jakob disease, Parkinson's disease, amyotrophic lateral sclerosis (ALS) disease, cognitive impairment, frontotemporal dementia (FTD), traumatic brain injury, postoperative cognitive dysfunction (PCD), neuro-otological syndromes, hearing loss, Huntington's disease, stroke, chronic traumatic encephalopathy, spinal cord injury, dementias or cognitive impairment, arthritis, psoriatic arthritis, psoriasis, juvenile idiopathic arthritis, asthma, allergic asthma, bronchial asthma, tuberculosis, chronic airway disorder, cystic fibrosis, glomerulonephritis, membranous nephropathy, sarcoidosis, vasculitis, ichthyosis, transplant rejection, interstitial cystitis, atopic dermatitis or inflammatory bowel disease, Crohn's disease, ulcerative colitis, celiac disease, systemic lupus erythematosus, type 1 diabetes, multiple sclerosis, rheumatoid arthritis, acute pancreatitis, chronic pancreatitis, alcoholic liver steatosis, obesity, glucose intolerance, insulin resistance, hyperglycemia, fatty liver, dyslipidemia, hyperlipidemia, type 2 diabetes, pancreatic cancer, breast cancer, kidney cancer, bladder cancer, prostate cancer, testicular cancer, urothelial cancer, endometrial cancer, ovarian cancer, cervical cancer, renal cancer, esophageal cancer, gastrointestinal stromal tumor (GIST), multiple myeloma, cancer of secretory cells, thyroid cancer, gastrointestinal carcinoma, chronic myeloid leukemia, hepatocellular carcinoma, colon cancer, melanoma, malignant glioma, glioblastoma, glioblastoma multiforme, astrocytoma, dysplastic gangliocytoma of the cerebellum, Ewing's sarcoma, rhabdomyosarcoma, ependymoma, medulloblastoma, ductal adenocarcinoma, adenosquamous carcinoma, nephroblastoma, acinar cell carcinoma, lung cancer, non-Hodgkin's lymphoma, Burkitt's lymphoma, chronic lymphocytic leukemia, monoclonal gammopathy of undetermined significance (MGUS), plasmocytoma, lymphoplasmacytic lymphoma, acute lymphoblastic leukemia, Pelizaeus-Merzbacher disease, atherosclerosis, abdominal aortic aneurism, carotid artery disease, deep vein thrombosis, Buerger's disease, chronic venous hypertension, vascular calcification, telangiectasia or lymphoedema, glaucoma, age-related macular degeneration, inflammatory retinal disease, retinal vascular disease, diabetic retinopathy, uveitis, rosacea, Sjogren's syndrome or neovascularization in proliferative retinopathy, hyperhomocysteinemia, skeletal muscle atrophy, myopathy, muscular dystrophy, muscular wasting, sarcopenia, Duchenne muscular dystrophy (DMD), Becker's disease, myotonic dystrophy, X-linked dilated cardiomyopathy, spinal muscular atrophy (SMA), Down syndrome, MEHMO syndrome, metaphyseal chondrodysplasia, Schmid type (MCDS), depression, or social behavior impairment.

19. A method of producing a protein, comprising contacting a eukaryotic cell comprising a nucleic acid encoding the protein with the compound or salt of claim 1.

20. The method of claim 19, comprising culturing the cell in an in vitro culture medium comprising the compound or salt.

21. A method of culturing a eukaryotic cell comprising a nucleic acid encoding a protein, comprising contacting the eukaryotic cell with an in vitro culture medium comprising a compound or salt of claim 1.

22. The method of claim 19, wherein the nucleic acid encoding the protein is a recombinant nucleic acid.

23. The method of claim 19, wherein the cell is a human embryonic kidney (HEK) cell or a Chinese hamster ovary (CHO) cell.

24. The method of claim 19, wherein the cell is a yeast cell, a wheat germ cell, an insect cell, a rabbit reticulocyte, a cervical cancer cell, a baby hamster kidney cell, a murine myeloma cell, an HT-1080 cell, a PER.C6 cell, a plant cell, a hybridoma cell, or a human blood derived leukocyte

25. A method of producing a protein, comprising contacting a cell-free protein synthesis (CFPS) system comprising eukaryotic initiation factor 2 (eIF2) and a nucleic acid encoding a protein with the compound or salt of claim 1.

26. The method of claim 19, wherein the protein is an antibody or a fragment thereof.

27. The method of claim 19, wherein the protein is a recombinant protein, an enzyme, an allergenic peptide, a cytokine, a peptide, a hormone, erythropoietin (EPO), an interferon, a granulocyte-colony stimulating factor (G-CSF), an anticoagulant, or a clotting factor.

28. The method of claim 19, comprising purifying the protein.

29. An in vitro cell culture medium, comprising the compound or salt of claim 1 and nutrients for cellular growth.

30. The cell culture medium of claim 29, comprising a eukaryotic cell comprising a nucleic acid encoding a protein.

31. The cell culture medium of claim 29, further comprising a compound for inducing protein expression.

32. The cell culture medium of claim 29, wherein the nucleic acid encoding the protein is a recombinant nucleic acid.

33. The cell culture medium of claim 29, wherein the protein is an antibody or a fragment thereof.

34. The cell culture medium of claim 29, wherein the protein is a recombinant protein, an enzyme, an allergenic peptide, a cytokine, a peptide, a hormone, erythropoietin (EPO), an interferon, a granulocyte-colony stimulating factor (G-CSF), an anticoagulant, or a clotting factor.

35. The cell culture medium of claim 29, wherein the eukaryotic cell is a human embryonic kidney (HEK) cell or a Chinese hamster ovary (CHO) cell.

36. The cell culture medium of claim 29, wherein the cell is a yeast cell, a wheat germ cell, an insect cell, a rabbit reticulocyte, a cervical cancer cell, a baby hamster kidney cell, a murine myeloma cell, an HT-1080 cell, a PER.C6 cell, a plant cell, a hybridoma cell, or a human blood derived leukocyte

37. A cell-free protein synthesis (CFPS) system comprising eukaryotic initiation factor 2 (eIF2) and a nucleic acid encoding a protein with the compound or salt of claim 1.

38. The CFPS system of claim 37, comprising a eukaryotic cell extract comprising eIF2.

39. The CFPS system of claim 37, further comprising eIF2B.

40. The CFPS system of claim 37, wherein the protein is an antibody or a fragment thereof.

41. The CFPS system of claim 37, wherein the protein is a recombinant protein, an enzyme, an allergenic peptide, a cytokine, a peptide, a hormone, erythropoietin (EPO), an interferon, a granulocyte-colony stimulating factor (G-CSF), an anticoagulant, or a clotting factor.
